(19) United States
(12) Patent Application Publication
(10) Pub. No.: US 2008/0293711 A1
(43) Pub. Date: Nov. 27, 2008

(54) Chemokine receptor modulators

(76) Inventors: Michael P. Clark, Duluth, GA (US); Mark A. Lockwood, Alpharett, GA (US); Florence F. Wagner, Duluth, GA (US); Michael G. Natchus, Alpharetta, GA (US); Brandon C. Doroh, Duluth, GA (US)

Correspondence Address: COOLEY GODWARD KRONISH LLP;ATTN: Patent Group, Suite 1100, 777 - 6th Street, NW, WASHINGTON, DC, 20001, US

(21) Appl. No.: 12/073,672

(22) Filed: Mar. 7, 2008

Related U.S. Application Data
(60) Provisional application No. 60/905,610, filed on Mar. 8, 2007.

(51) Int. Cl. A61K 31/5377; C07D 417/12; A61K 31/16; A61P 35/00; C07C 243/12

(52) U.S. Cl. 514/231.5; 544/146; 564/149; 514/615

(57) ABSTRACT

The invention provides compounds of Formula (I)

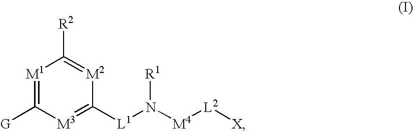

and pharmaceutical compositions comprising compounds of Formula (I). These compounds are useful treating or preventing HIV infections, and in treating proliferative disorders such as inhibiting the metastasis of various cancers.

### CROSS-REFERENCE TO RELATED APPLICATIONS

The present application claims priority to U.S. Provisional Application No. 60/905,610 filed Mar. 8, 2007, the disclosure of which is herein incorporated by reference in its entirety for all purposes.

### FIELD OF THE INVENTION

The invention provides compounds, pharmaceutical compositions and methods of use of certain compounds that are modulators of chemokine receptors. The compounds are useful to modulate a medical condition that is modulated by chemokine receptor activity or signaling, and in particular in the treatment or prevention of human immunodeficiency virus infections (HIV) or the diagnosis, prevention, and treatment of cancer.

### BACKGROUND

Cancer is currently the second leading cause of death in developed nations. In 2004, the American Cancer Society estimated that approximately 1.37 million new cases were diagnosed in the U.S. alone, and approximately 550,000 deaths occurred due to cancer (American Cancer Society, Cancer Facts & Figures 2004, see URL: http://www.cancer.org/docroot/STT/stt_{—}0.asp).

Metastasis, the spread and growth of tumor cells to distant organs, is the most devastating attribute of cancer. Most morbidity and mortality associated with certain types of cancer, such as breast cancer, is associated with disease caused by metastatic cells rather than by the primary tumor. Therapy for metastasis currently relies on a combination of early diagnosis and aggressive treatment of the primary tumor.

The establishment and growth of metastases at distant sites is thought to depend on interactions between tumor cells and the host environment. Metastasis as the result of several sequential steps and represents a highly organized, non-random and organ-selective process. Although a number of mediators have been implicated in the metastasis of breast cancer, the precise mechanisms determining the directional migration and invasion of tumor cells into specific organs remain to be established. An incomplete understanding of the molecular and cellular mechanisms underlying metastasis has hindered the development of effective therapies that would eliminate or ameliorate this condition.

Several strategies have been developed to reduce metastatic invasion of malignant cells by regulating adhesion of endothelial cells with antibodies or adhesion molecules (see for example, PCT Publication No. WO 97100956, U.S. Pat. Nos. 5,993,817; 6,433,149; 6,475,488; and 6,358,915). However no commercial strategy has provided an effective treatment to prevent metastasis.

Chemokines are considered to be principal mediators in the initiation and maintenance of inflammation. They have also been found to play an important role in the regulation of endothelial cell function, including proliferation, migration and differentiation during angiogenesis and re-endothelialization after injury (Gupta et al. (1998) J Biol Chem, 7:4282-4287). Two specific chemokines have also been implicated in the etiology of infection by human immunodeficiency virus (HIV).

As of the end of 2004, an estimated 39.4 million people worldwide were living with HIV/AIDS, and the Centers for Disease Control and Prevention (CDC) estimate that 850,000 to 950,000 U.S. residents are living with HIV infection (UNAIDS/WHO AIDS epidemic update, December 2004; Fleming, P. L. et al. HIV Prevalence in the United States, 2000. 9^{th} Conference on Retroviruses and Opportunistic Infections, Seattle, Wash., Feb. 24-28, 2002. Abstract 11). Although new infections have decreased in recent years, an estimated 4.9 million new HIV infections occurred worldwide during 2004 and approximately 40,000 new HIV infections occur each year in the United States.

HIV entry within the target cells involves a series of molecular events. The three main steps of virus entry within the cell are: (i) attachment of the virus to the host cells; (ii) interaction of the virus with the co-receptors; (iii) fusion of the virus and host cell membranes. Considering the complexity of the molecular events involved in viral infection, all three of these steps have been considered for the drug design of HIV entry inhibitors. The T-lymphocyte cell surface protein CD4 is the primary receptor involved in the interaction with the viral glycoprotein gp120, but a cellular co-receptor is also needed for the successful entry of the virus within the cell. At least two types of such co-receptors have been identified so far, both of which are chemokine receptors. These chemokine receptors are therefore gateways for HIV entry, determinants of viral tropism and sensitivity.

Chemokines are a superfamily of small, secreted cytokines that induce, through their interaction with G-protein-coupled receptors, cytoskeletal rearrangements and directional migration of several cell types (Butcher, et al. (1999) Adv Immunol 72: 209-253; Campbell and Butcher (2000) Curr Opin Immunol 12: 336-341; Zlotnik and Yoshie (2000) Immunity 12: 121-127). The chemokine receptor, CXCR4, is known in viral research as a major coreceptor for the entry of T cell line-tropic HIV (Feng, et al. (1996) Science 272: 872-877; Davis, et al. (1997) J Exp Med 186: 1793-1798; Zaitseva, et al. (1997) Nat Med 3: 1369-1375; Sanchez, et al. (1997) J Biol Chem 272: 27529-27531). T Stromal cell derived factor 1 (SDF-1) is a chemokine that interacts specifically with CXCR4. When SDF-1 binds to CXCR4, CXCR4 activates Gal-protein-mediated signaling (pertussis toxin-sensitive) (Chen, et al. (1998) Mol Pharmacol 53: 177-181), including downstream kinase pathways such as Ras/MAP Kinases and phosphatidylinositol 3-kinase (PI3K)/Akt in lymphocyte, megakaryocytes, and hematopoietic stem cells (Bleul, et al. (1996) Nature 382: 829-833; Deng, et al. (1997) Nature 388: 296-300; Kijowski, et al. (2001) Stem Cells 19: 453-466; Majka, et al. (2001) Folia. Histochem. Cytobiol. 39: 235-244; Sotsios, et al. (1999) J. Immunol. 163: 5954-5963; Vlahakis, et al. (2002) J. Immunol. 169: 5546-5554).

Compounds targeting CXCR4 have been developed primarily for treatment of HIV because CXCR4 is a major coreceptor for T-tropic HIV infection. For example, U.S. Pat. No. 6,429,308 to Hisamitsu Pharmaceutical Co., Inc. discloses an antisense oligonucleotide to CXCR4 to inhibit the expression of the CXCR4 protein for use as an anti-HIV agent. PCT Publication No. WO 01156591 to Thomas Jefferson University describes peptide fragments of viral macrophage inflammatory protein II which are described as selectively preventing CXCR4 signal transduction and coreceptor function in mediating entry of HIV-1.

Peptide antagonists of CXCR4 receptors have also been disclosed. Tamamura et al (Tamamura, et al. (2000) Bioorg. Med. Chem. Lett. 10: 2633-2637; Tamamura, et al. (2001) Bioorg. Med. Chem. Lett. 11: 1897-1902) reported the identification of a specific peptide-based CXCR4 inhibitor, T140. T140 is a 14-residue peptide that possessed high levels of anti-HIV activity and antagonism of T cell line-tropic HIV-1 entry among all antagonists of CXCR4 (Tamamura, et al. (1998) Biochem. Biophys. Res. Commun. 253: 877-882). The compound has been altered to increase its efficacy and bioavailability by, for example, amidating the C-terminal of T-140 and reducing the total positive charges by substituting basic residues with nonbasic polar amino acids to generate TN14003, which is less cytotoxic and more stable in serum compared to T140. The concentration of TN14003 required for 50% protection of HIV-induced cytopathogenicity in MT-4 cells is 0.6 nM in contrast to 410 mM leading to 50% toxicity. U.S. Pat. No. 6,344,545 to Progenics Pharmaceuticals, Inc. describes methods for preventing HIV-1 infection of CD4+ cells with peptide fragments. U.S. Pat. No. 6,534,626 to the U.S. Department of Health & Human Services describes certain peptide chemokine variants for treating HIV infections. PCT Publication No. WO 041087068 to Emory University describes CXCR4 peptide antagonists, particularly TN14003, and methods of their use to treat metastasis.

Other peptide-based antagonists have also been disclosed. For example, European Patent Publication Nos. 1 286 684 and 1 061 944 to the University Of British Columbia cover methods of treatment of diseases, including metastasis, using modified peptide CXCR4 antagonists derived from the native SDF-1 ligand. PCT Publication No. WO 041020462 to Takeda Chemical Industries, Ltd. provides peptide CXCR4 antagonists for treatment and prevention of breast cancer and chronic rheumatoid arthritis. U.S. Patent Application No. 200410132642 to the U.S. Dept. of Health & Human Services in part covers methods of inhibiting metastasis or growth of a tumor cell with a polypeptide CXCR4 inhibitor.

In mice transplanted with human lymph nodes, SDF-1 induces CXCR4-positive cell migration into the transplanted lymph node (Blades et al. (2002) J. Immunol. 168: 4308-4317). These results imply that the interaction between SDF-1 and CXCR4 directs cells to the organ sites with high levels of SDF-1.

Recently, studies have shown that CXCR4 interactions may regulate the migration of metastatic cells. Hypoxia, a reduction in partial oxygen pressure, is a microenvironmental change that occurs in most solid tumors and is a major inducer of tumor angiogenesis and therapeutic resistance. Hypoxia increases CXCR4 levels (Staller, et al. (2003) Nature 425: 307-31 1). Microarray analysis on a sub-population of cells from a bone metastatic model with elevated metastatic activity showed that one of the genes increased in the metastatic phenotype was CXCR4. Furthermore, overexpression of CXCR4 in isolated cells significantly increased the metastatic activity (Kang, et al. (2003) Cancer Cell 3: 537-549). In samples collected from various breast cancer patients, Muller et al. (Muller, et al. (2001) Nature 410: 50-56) found that CXCR4 expression level is higher in primary tumors relative to normal mammary gland or epithelial cells. These results suggest that the expression of CXCR4 on cancer cell surfaces may direct the cancer cells to sites that express high levels of SDF-I. Consistent with this hypothesis, SDF-1 is highly expressed in the most common destinations of breast cancer metastasis including lymph nodes, lung, liver, and bone marrow. Moreover, CXCR4 antibody treatment has been shown to inhibit metastasis to regional lymph nodes when compared to control isotypes that all metastasized to lymph nodes and lungs (Muller, et al. (2001)).

In addition to regulating migration of cancer cells, CXCR4-SDF-1 interactions may regulate vascularization necessary for metastasis. Blocking either CXCR4/SDF-1 interaction or the major G-protein of CXCR4/SDF-1 signaling pathway (Gα₁) inhibits VEGF-dependent neovascularization. These results indicate that SDF-1/CXCR4 controls VEGF signaling systems that are regulators of endothelial cell morphogenesis and angiogenesis. Numerous studies have shown that VEGF and MMPs actively contribute to cancer progression and metastasis.

Several groups have identified chemokines including CXCR4 as a target for treatment of metastatic cancers. For example, PCT Publication Nos. WO 01138352 to Schering Corporation, WO 041059285 to Protein Design Labs, Inc., and WO 041024178 to Burger generally describe methods of treating diseases and specifically inhibiting metastasis by blocking chemokine receptor signaling.

Although advances have been made, inadequate absorption, distribution, metabolism, excretion or toxicity properties of peptide inhibitors have limited their clinical use. Small non-peptide drugs remain a major goal of medicinal chemistry programs in this area.

At the present time, the metal-chelating cyclams and bicyclams represent one of the few reported non-peptide molecules to effectively block CXCR4 (Onuffer and Horuk (2002) Trends Pharmacol Sci 23: 459-467.36). One of these non-peptide molecules is AMD3100, which entered clinical trials as an anti-HIV drug that blocks CXCR4-mediated viral entry (Donzella, et al. (1998) Nat Med 4: 72-77; Hatse, et al. (2002) FEBS Lett 527: 255-262; Fujii, et al. (2003) Expert Opin Investig Drugs 12: 185-195; Schols, et al. (1997) Antiviral Res 35: 147-156).

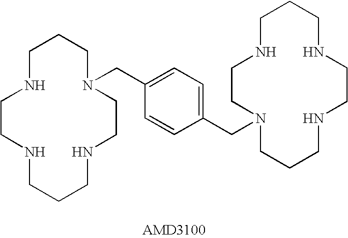

However, a clinical study showed cardiac-related side effect of AMD3100 (Scozzafava, et al. (2002) J Enzyme Inhib Med Chem 17: 69-7641). In fact, AMD3100, was recently withdrawn from the clinical trials due in part to a cardiac-related side effect (Hendrix, et al. (2004) Journal of Acquired Immune Deficiency Syndromes 37(2)). The latter was not a result of the compound's ability to block CXCR4 function, but due to its presumed structural capacity for encapsulating metals.

Other nitrogen containing bicyclic molecules have also been developed as CXCR4 antagonists. European Patent Publication No. 1 431 290 and PCT Publication No. WO 02/094261 to Kureha Chemical Industry Co., Ltd cover CXCR4 inhibitors that are potentially useful in treating various diseases including HIV infection.

U.S. Patent Publication No. 2004/0254221 to Yamamazi, et al. also provides compounds and use thereof to treat various diseases including HIV infections that are CXCR4 antagonists. The compounds are of the general formula:

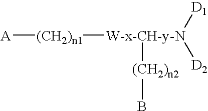

in which A is A₁-G₁-N(R₁)—; A₁ is hydrogen or an optionally substituted, mono- or polycyclic, heteroaromatic or aromatic ring; G₁ is a single bond or —C(R₂)(R₃)—; R₁, R₂, and R₃ can be optionally substituted hydrocarbon groups; W is an optionally substituted hydrocarbon or heterocyclic ring; x is —C(.O)NH—; y is —C(.O)—; and D₁ is hydrogen atom, alkyl with a polycyclic aromatic ring, or amine.

PCT Publication No. WO 00/56729 and U.S. Pat. No. 6,750,348 to AnorMED describe certain heterocyclic small molecule CXCR4 binding compounds, teaching that these are useful for the protection against HIV infection. The compounds are of the general formula:

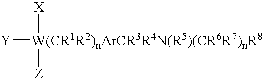

in which W can be a nitrogen or carbon atom; Y is absent or is hydrogen; R¹ to R⁷ can be hydrogen or straight, branched or cyclic C₁₋₆ alkyl; R⁸ is a substituted heterocyclic or aromatic group; Ar is an aromatic or heteroaromatic ring; and X is specified ring structure.

PCT Publication No. WO 2004/091518 to AnorMED also describes certain substituted nitrogen containing compounds that bind to CXCR4 receptors. The compounds are described as having the effect of increasing progenitor cells and/or stem cells, enhancing production of white blood cells, and exhibiting antiviral properties. PCT Publication No. WO 2004/093817 to AnorMED also discloses substituted heterocyclic CXCR4 antagonists which are described as useful to alleviate inflammatory conditions and elevate progenitor cells, as well as white blood cell counts. Similarly, PCT Publication No. WO 2004/106493 to AnorMED describes heterocyclic compounds that bind to CXCR4 and CCR5 receptors consisting of a core nitrogen atom surrounded by three pendant groups, wherein two of the three pendant groups are preferably benzimidazolyl methyl and tetrahydroquinolyl, and the third pendant group contains nitrogen and optionally contains additional rings. The compounds demonstrate protective effects against infections of target cells by a human immunodeficiency virus (HIV).

PCT Patent Application PCT/US06/000604, filed Jan. 9, 2006, describes certain compounds for the treatment of medical disorders mediated by CXCR4. These compounds include two nitrogen linked cyclic substituents off a central aromatic or cyclic alkyl or heteroalkyl.

In light of the fact that the chemokine receptors are implicated in metastatic signaling as well as a number of other pathogenic conditions, it is important to identify new effective chemokine receptor modulators.

It is therefore an object of the invention to provide new compounds, methods and compositions that modulate chemokine receptors.

It is another object of the invention to provide compounds, methods and compositions that bind to chemokine receptors and interfere with binding to their native ligands.

It is an object of the invention to provide new compounds, methods and compositions for the treatment of viral infections, such as HIV.

It is also an object of the invention to provide compounds, methods, and compositions for treatment of proliferative disorders, such as for the inhibition of cancer metastases.

### SUMMARY

In one embodiment, the compounds of the present invention are compounds of formula (I), or a pharmaceutically acceptable salt, solvate, prodrug, enantiomer, diastereomer, tautomer, or ester thereof:

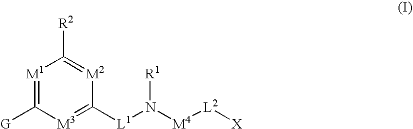

M¹ is ═N— or ═C(G)-;
M² and M³ are each independently ═N— or ═C(R³)—;
M⁴ is —C(R⁴)₂— or —N(R⁵)—;
each G is independently H, -L³-N(R⁶)-L⁴-Y, or a substituted or unsubstituted heterocyclyl of formula A:

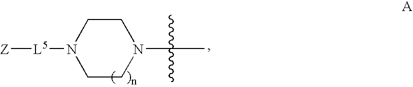

wherein n is an integer ranging from 1-3;
L¹ is selected from the group consisting of a covalent bond, —C(R⁴)₂—, —C(O)—, —S(O)₂—, —S(O)—, and —C(R⁴)═;
L² is selected from the group consisting of a covalent bond, —S(O)₂—, —S(O)—, —C(O)—, —C(O)O—, —C(R⁴)₂—, and —C(R⁴)═;
L³ is selected from the group consisting of —C(O)—, —C(R⁴)₂—, —S(O)₂—, and —S(O)—;
L⁴ is selected from the group consisting of a covalent bond, —C(R⁴)₂—, —C(O)—, —S(O)₂—, and —S(O)—;
L⁵ is selected from the group consisting of a covalent bond, —C(R⁴)₂—, —C(O)—, —S(O)₂—, and —S(O)—;
X is selected from the group consisting of H, alkyl, substituted or unsubstituted aryl, substituted or unsubstituted arylalkyl, substituted or unsubstituted heteroaryl, substituted or unsubstituted heterocyclyl, substituted or unsubstituted cycloalkyl, substituted or unsubstituted amino, substituted or unsubstituted aminoalkyl, and substituted or unsubstituted amido;
Y is selected from the group consisting of substituted or unsubstituted heteroaryl, substituted or unsubstituted aryl, hydroxyalkyl, alkoxyalkyl, substituted or unsubstituted aminoalkyl, substituted or unsubstituted cycloalkyl, substituted or unsubstituted —C(O)-heterocyclyl, substituted or unsubstituted amino, substituted or unsubstituted arylalkylamino, substituted or unsubstituted heteroarylalkylamino, amidoalkyl, substituted or unsubstituted amido, substituted or unsubstituted heterocyclyl, and alkyl;
Z is selected from the group consisting of H, alkyl, substituted or unsubstituted aryl, substituted or unsubstituted heteroaryl, alkoxyalkyl, and hydroxyalkyl;
R¹ is selected from the group consisting of H, alkyl, alkenyl, aminoalkyl, substituted or unsubstituted arylalkyl, substituted or unsubstituted —S(O)₂-aryl, substituted or unsubstituted —S(O)₂-heteroaryl, substituted or unsubstituted heteroarylalkyl, substituted or unsubstituted cycloalkyl, substituted or unsubstituted aryl, substituted or unsubstituted heteroaryl, acyl, carboxy, alkoxycarbonyl, and substituted or unsubstituted aminocarbonyl;
R² and each R³ are each independently selected from the group consisting of H, alkyl, halo, haloalkyl, alkoxy, hydroxy, acyl, substituted or unsubstituted amino, alkyl-S(O)ₘ—, substituted or unsubstituted amino-S(O)ₘ—, substituted or unsubstituted amido, carboxy, alkoxycarbonyl, substituted or unsubstituted aminocarbonyl, and —CN, wherein m is an integer ranging from 0-2;
each R⁴ is independently selected from the group consisting of H, alkyl, alkylthioalkyl, thioalkyl, substituted or unsubstituted aryl, substituted or unsubstituted aminoalkyl, and substituted or unsubstituted heteroaryl;
R⁵ is selected from the group consisting of H, substituted or unsubstituted heteroarylalkyl, alkyl, substituted or unsubstituted aminoalkyl, alkyl-S(O)₂—, substituted or unsubstituted cycloalkyl, substituted or unsubstituted arylalkyl, substituted or unsubstituted aryl, substituted or unsubstituted heteroaryl, substituted or unsubstituted heteroarylalkyl, acyl, carboxy, alkoxycarbonyl, and substituted or unsubstituted aminocarbonyl; or
R⁵ and R¹, together with the nitrogen atoms to which they are each attached, form a substituted or unsubstituted heterocyclyl ring; or
R⁵ and -L²-X, together with the nitrogen atom to which they are both attached, form a substituted or unsubstituted heterocyclyl ring;
R⁶ is selected from the group consisting of H, alkyl, substituted or unsubstituted aminoalkyl, substituted or unsubstituted arylalkyl, substituted or unsubstituted cycloalkyl, alkoxyalkyl, hydroxyalkyl, substituted or unsubstituted heteroarylalkyl, substituted or unsubstituted aryl, substituted or unsubstituted heteroaryl, acyl, carboxy, alkoxycarbonyl, and substituted or unsubstituted aminocarbonyl; or
R⁶ and -L⁴-Y, together with the nitrogen atom to which they are both attached, form a substituted or unsubstituted heterocyclyl ring;
with the following provisos:
   (i) at least one G is not H;
   (ii) when M⁴ is —N(R⁵)— and L² is —C(R⁴)═, then -M⁴-L²- is —N═C(R⁴)—;
   (iii) when M⁴ is —C(R⁴)₂— and L² is —C(R⁴)═, then —N(R¹)-M⁴-L²- is —N(R¹)C(R⁴)═C(R⁴)—;
   (iv) when L¹ is —C(R⁴)═, then -L¹-N(R¹)— is —C(R⁴)═N—;
   (v) when M⁴ is —N(R⁵)— and G is a selected from the group consisting of substituted or unsubstituted piperazine, substituted or unsubstituted —CH₂-piperazine, and a substituted —CH₂-morpholine group, then X is not a 4-pyrimidinyl-2-cyano group; and
   (vi) when M⁴ is —C(R⁴)₂—, then L¹ and L³ are both —C(O)— and X is not a substituted or unsubstituted imidazolyl or substituted or unsubstituted indolyl, or then at least one group G is a substituted or unsubstituted heterocyclyl of formula A.

In a another embodiment, the compounds of the present invention, or pharmaceutically acceptable salts, solvates, prodrugs, tautomers, or esters thereof have the structure of formula (IA):

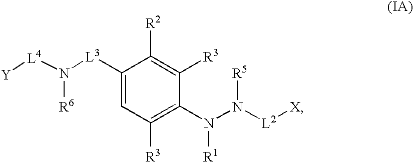

wherein L², L³, L⁴, R¹, R², each R³, R⁵, R⁶, X and Y are as defined herein.

In another embodiment, the compounds of the present invention, or pharmaceutically acceptable salts, solvates, prodrugs, tautomers, or esters thereof have the structure of formula (IB):

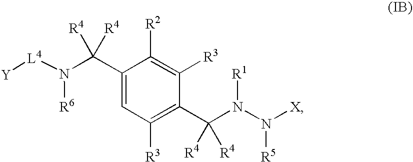

wherein L², R³, R⁴, each R³, each R⁴, R⁵, R⁶, X and Y are as defined herein.

In another embodiment, the compounds of the present invention, or pharmaceutically acceptable salts, solvates, prodrugs, tautomers, or esters thereof have the structure of formula (IC):

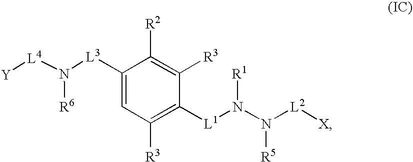

wherein L¹, L², L³, L⁴, R¹, R², each R³, R⁵, R⁶, X and Y are as defined herein.

In another embodiment, the compounds of the present invention, or pharmaceutically acceptable salts, solvates, prodrugs, tautomers, or esters thereof have the structure of formula (ID):

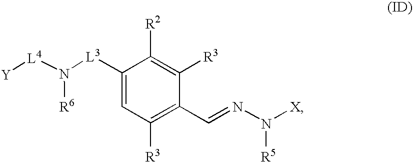

wherein L³, L⁴, R², each R³, R⁵, R⁶, X and Y are as defined herein.

In another embodiment, the compounds of the present invention, or pharmaceutically acceptable salts, solvates, prodrugs, tautomers, or esters thereof have the structure of formula (IE):

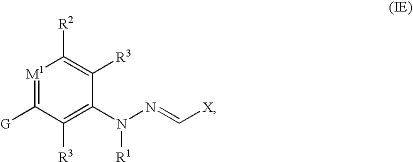

wherein R¹, R², each R³, M¹, and G are as defined herein.

In another embodiment, the compounds of the present invention, or pharmaceutically acceptable salts, solvates, prodrugs, tautomers, or esters thereof have the structure of formula (IF):

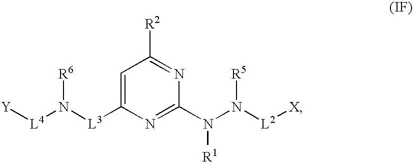

wherein L², L³, L⁴, R¹, R², R⁵, R⁶, X and Y are as defined herein.

In another embodiment, the compounds of the present invention, or pharmaceutically acceptable salts, solvates, prodrugs, tautomers, or esters thereof have the structure of formula (IG):

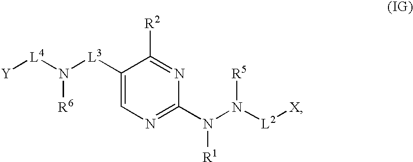

wherein L², L³, L⁴, R¹, R², R⁵, R⁶, X and Y are as defined herein.

In another embodiment, the compounds of the present invention, or pharmaceutically acceptable salts, solvates, prodrugs, tautomers, or esters thereof have the structure of formula (IH):

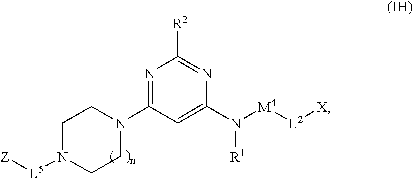

wherein L², L⁵, M⁴, R¹, R², n, X and Y are as defined herein.

In another embodiment, the compounds of the present invention, or pharmaceutically acceptable salts, solvates, prodrugs, tautomers, or esters thereof have the structure of formula (II):

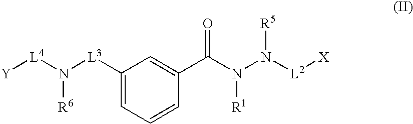

wherein L², L³, L⁴, R¹, R⁵, X and Y are as defined herein.

In yet another embodiment, the present invention is directed to a pharmaceutical composition comprising at least one compound of formula (I), or a pharmaceutically acceptable salt, solvate, prodrug, tautomers, or ester thereof, and a pharmaceutically acceptable excipient.

In yet another embodiment, the present invention is directed to a pharmaceutical composition comprising at least one compound of formula (I), or a pharmaceutically acceptable salt, solvate, prodrug, tautomers, or ester thereof, a pharmaceutically acceptable excipient, and at least one additional pharmaceutically active compound.

In still another embodiment, the present invention is directed to a method of treating a disorder, symptom or disease in a patient, comprising administering to the patient an effective amount of at least one compound of formula (I).

In still yet another embodiment, the present invention is directed to treatment or prophylaxis of a disorder, symptom or disease that is modulated by chemokine receptor activity or signaling.

### DETAILED DESCRIPTION OF THE INVENTION

The compounds, methods, and compositions of the present invention are chemokine receptor modulators. These compounds can be used to treat or prevent HIV infection, reduce viral load, or alleviate progression towards, or the symptoms of AIDS in a host in need thereof. In addition, these compounds can be used to treat tumor metastasis or any other disease, particularly hyperproliferative diseases involving chemokine receptors.

Compounds described herein have the capacity to interact with chemokine receptors and potentially inhibit receptor signaling. The compounds of the present invention have increased bioavailability and efficacy in inhibiting chemokine receptors.

Although not bound by theory, these compounds may inhibit metastasis through their capacity to modulate chemokine receptors, which can decrease cell targeting, and may also reduce VEGF-dependent endothelial cell morphogenesis and angiogenesis. This endothelial cell growth is a key event in metastases of tumors.

In one embodiment, the compounds of the present invention, or pharmaceutically acceptable salts, solvates, prodrugs, tautomers, or esters thereof, have the structure of formula (I) as described herein.

In another embodiment, the compounds of the present invention, or pharmaceutically acceptable salts, solvates, prodrugs, tautomers, or esters thereof, have the structure of formula (IA):

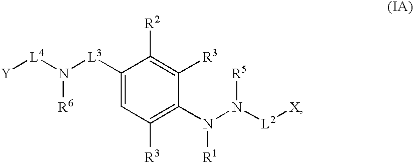

wherein R¹, R², each R³, R⁵, R⁶, L², L³, L⁴, X and Y are independently as defined herein.

In another embodiment of the compounds of formula (IA),
L² is selected from the group consisting of —S(O)₂—, —S(O)—, —C(O)—, and —C(R⁴)₂—;
L³ is selected from the group consisting of —C(O)—, —C(R⁴)₂—, —S(O)₂—, and —S(O)—;
L⁴ is —C(R⁴)₂— or —C(O);
X is selected from the group consisting of substituted or unsubstituted aryl, substituted or unsubstituted heteroaryl, and substituted or unsubstituted cycloalkyl;
Y is selected from the group consisting of substituted or unsubstituted heteroaryl, substituted or unsubstituted aryl, hydroxyalkyl, alkoxyalkyl, substituted or unsubstituted amidoalkyl, substituted or unsubstituted amido, substituted or unsubstituted heterocyclyl, and substituted or unsubstituted aminoalkyl;
R¹ is selected from the group consisting of H, alkyl, substituted or unsubstituted arylalkyl, substituted or unsubstituted heteroarylalkyl, substituted or unsubstituted cycloalkyl, substituted or unsubstituted aryl, substituted or unsubstituted heteroaryl, acyl, carboxy, alkoxycarbonyl, and aminocarbonyl;
R² and each R³ are each independently selected from the group consisting of H, alkyl, halo, haloalkyl, alkoxy, hydroxy, acyl, substituted or unsubstituted amino, alkyl-S(O)ₘ—, substituted or unsubstituted amino-S(O)ₘ—, substituted or unsubstituted amido, carboxy, alkoxycarbonyl, aminocarbonyl, and —CN, wherein m is an integer ranging from 0-2;
each R⁴ is independently selected from the group consisting of H, alkyl, alkylthioalkyl, thioalkyl, substituted or unsubstituted aryl, and substituted or unsubstituted heteroaryl;
R⁵ is selected from the group consisting of H, substituted or unsubstituted heteroarylalkyl, alkyl, substituted or unsubstituted cycloalkyl, substituted or unsubstituted arylalkyl, substituted or unsubstituted aryl, and substituted or unsubstituted heteroaryl; or
R⁵ and R¹, together with the nitrogen atoms to which they are each attached, form a substituted or unsubstituted heterocyclyl ring; and
R⁶ is selected from the group consisting of H, aminoalkyl, substituted or unsubstituted arylalkyl, substituted or unsubstituted cycloalkyl, alkoxyalkyl, hydroxyalkyl, substituted or unsubstituted heteroarylalkyl, substituted or unsubstituted aryl, and substituted or unsubstituted heteroaryl.

In another embodiment of the compounds of formula (IA), L² and L³ are both —C(O)—.

In another embodiment of the compounds of formula (IA), L² is —C(O)— and L³ is —C(R⁴)₂—.

In another embodiment of the compounds of formula (IA), L² is —C(O)— and L³ is —CH₂—.

In another embodiment of the compounds of formula (IA), L² is —S(O)₂— or —S(O)— and L³ is —C(O)—.

In another embodiment of the compounds of formula (IA), L² is —S(O)₂— and L³ is —C(R⁴)₂—.

In another embodiment of the compounds of formula (IA), L² is —S(O)₂— and L³ is —CH₂—.

In another embodiment of the compounds of formula (IA), L² is —S(O)₂—.

In another embodiment of the compounds of formula (IA), X is substituted or unsubstituted phenyl.

In another embodiment of the compounds of formula (IA), X and Y are independently substituted or unsubstituted aryl, or substituted or unsubstituted heteroaryl.

In another embodiment of the compounds of formula (IA), X and Y are independently selected from the group consisting of substituted or unsubstituted phenyl, substituted or unsubstituted pyridyl substituted or unsubstituted pyrimidyl, substituted or unsubstituted indolyl, substituted or unsubstituted benzimidazolyl, and substituted or unsubstituted pyrrolopyrimidyl.

In another embodiment of the compounds of formula (IA), Y is substituted or unsubstituted phenyl.

In another embodiment of the compounds of formula (IA), X and Y are both substituted or unsubstituted phenyl.

In another embodiment of the compounds of formula (IA), Y is 2-pyridyl.

In another embodiment of the compounds of formula (IA), X is substituted or unsubstituted phenyl and Y is 2-pyridyl.

In another embodiment of the compounds of formula (IA), Y is aminoalkyl.

In another embodiment of the compounds of formula (IA), X is substituted or unsubstituted aryl and Y is aminoalkyl.

In another embodiment of the compounds of formula (IA), R⁶ and Y are both aminoalkyl.

In another embodiment of the compounds of formula (IA), Y-L⁴- and R⁶ are both aminoalkyl.

In another embodiment of the compounds of formula (IA), Y-L⁴- and R⁶ are both —CH₂CH₂N(CH₃)₂.

In another embodiment of the compounds of formula (IA), Y-L⁴- and R⁶ are both —CH₂CH₂N(CH₃)₂ and L³ is —C(O)—.

In another embodiment of the compounds of formula (IA), L⁴ is —C(R⁴)₂—.

In another embodiment of the compounds of formula (IA), L⁴ is —CH₂—.

In another embodiment of the compounds of formula (IA), L³ and L⁴ are both —C(R⁴)₂—.

In another embodiment of the compounds of formula (IA), L³ and L⁴ are both —CH₂—.

In another embodiment of the compounds of formula (IA), L³ is —C(O)— and L⁴ is —C(R⁴)₂—.

In another embodiment of the compounds of formula (IA), L³ is —C(O)— and L⁴ is —CH₂—.

In another embodiment of the compounds of formula (IA), L³ is —C(O)—, L⁴ is —C(R⁴)₂—, and Y is substituted or unsubstituted phenyl.

In another embodiment of the compounds of formula (IA), L³ is —C(O)—, L⁴ is —C(R⁴)₂—, and Y is substituted or unsubstituted 2-pyridyl.

In another embodiment of the compounds of formula (IA), L³ is —C(R⁴)₂—, and L⁴ is a covalent bond.

In another embodiment of the compounds of formula (IA), L³ is —CH₂—, and L⁴ is a covalent bond.

In another embodiment of the compounds of formula (IA), L³ is —C(O)—, and L⁴ is a covalent bond.

In another embodiment of the compounds of formula (IA), L³ is —CH₂—, L⁴ is a covalent bond, and Y is a substituted or unsubstituted heteroaryl.

In another embodiment of the compounds of formula (IA), L³ is —CH₂—, L⁴ is a covalent bond, and Y is a substituted or unsubstituted pyrimidyl.

In another embodiment of the compounds of formula (IA), L³ is —CH₂—, L⁴ is a covalent bond, and Y is a substituted or unsubstituted 2-pyridyl.

In another embodiment of the compounds of formula (IA), L³ is —C(O)—, L⁴ is a covalent bond, and Y is a substituted or unsubstituted heteroaryl.

In another embodiment of the compounds of formula (IA), L³ is —C(O)—, L⁴ is a covalent bond, and Y is a substituted or unsubstituted 2-pyridyl.

In another embodiment of the compounds of formula (IA), L³ is —C(O)—, L⁴ is a covalent bond, and Y is a substituted or unsubstituted pyrimidyl.

In another embodiment of the compounds of formula (IA), L³ is —CH₂—, L⁴ is a covalent bond, and Y is substituted or unsubstituted phenyl.

In another embodiment of the compounds of formula (IA), L³ and L⁴ are both —C(R⁴)₂— and Y is alkoxyalkyl.

In another embodiment of the compounds of formula (IA), L³ and L⁴ are both —CH₂— and Y is alkoxyalkyl.

In another embodiment of the compounds of formula (IA), L³ and L⁴ are both —CH₂— and Y is CH₃—O—CH₂—.

In another embodiment of the compounds of formula (IA), L³ is —C(O)—, L⁴ is —C(R⁴)₂—, and Y is substituted or unsubstituted quinolinyl.

In another embodiment of the compounds of formula (IA), L² is —C(O)—.

In another embodiment of the compounds of formula (IA), L² is —S(O)₂—.

In another embodiment of the compounds of formula (IA), L² is —C(R⁴)₂—.

In another embodiment of the compounds of formula (IA), L² is —C(O)— and R¹ and R⁵ are both H.

In another embodiment of the compounds of formula (IA), L² is —C(O)— and R¹ is H.

In another embodiment of the compounds of formula (IA), L² is —C(O)—, R¹ is H, and R⁵ is substituted or unsubstituted arylalkyl.

In another embodiment of the compounds of formula (IA), L² is —C(O)—, R¹ is H, and R⁵ is substituted or unsubstituted benzyl.

In another embodiment of the compounds of formula (IA), L² is —C(O)—, R¹ is H, and R⁵ is substituted or unsubstituted heteroarylalkyl.

In another embodiment of the compounds of formula (IA), L² is —C(O)—, R¹ is H, and R⁵ is substituted or unsubstituted benzimidazolyl.

In another embodiment of the compounds of formula (IA), R⁵ and R¹, together with the nitrogen atoms to which they are each attached, form a substituted or unsubstituted heterocyclyl ring.

In another embodiment of the compounds of formula (IA), R⁵ and R¹, together with the nitrogen atoms to which they are each attached, form a substituted or unsubstituted 5-membered ring.

In another embodiment of the compounds of formula (IA), R⁵ and R¹, together with the nitrogen atoms to which they are each attached, form a substituted or unsubstituted 6-membered ring.

In another embodiment of the compounds of formula (IA), R⁵ and R¹, together with the nitrogen atoms to which they are each attached, form a substituted or unsubstituted pyrazolidine ring.

In another embodiment of the compounds of formula (IA), R⁵ and R¹, together with the nitrogen atoms to which they are each attached, form a substituted or unsubstituted piperazine ring.

In another embodiment of the compounds of formula (IA), L² is —S(O)₂—, R¹ and R⁵ are both H, and X is a substituted or unsubstituted phenyl.

In another embodiment of the compounds of formula (IA), L² is —C(O)—, R¹ and R⁵ are both H, and X is a substituted or unsubstituted phenyl.

In another embodiment of the compounds of formula (IA), L² is —C(O)—, R¹ and R⁵ are both H, and X is a substituted or unsubstituted pyrimidyl.

In another embodiment of the compounds of formula (IA), R⁵ and R¹, together with the nitrogen atoms to which they are each attached form a substituted or unsubstituted pyrazolidine ring, and L² is —C(O)—.

In another embodiment of the compounds of formula (IA), L² is —C(O)—, R¹ and R⁵ are both H, and X is a substituted or unsubstituted indolyl.

In another embodiment of the compounds of formula (IA), L² is —C(O)—, R¹ and R⁵ are both H, and X is a substituted or unsubstituted purine.

In another embodiment of the compounds of formula (IA), L² is —C(O)—, R¹ and R⁵ are both H, and X is a substituted or unsubstituted deazapurine.

In another embodiment of the compounds of formula (IA), L² is —S(O)₂—, X is a substituted or unsubstituted phenyl, R¹ is H, and R⁵ is a substituted or unsubstituted heteroarylalkyl.

In another embodiment of the compounds of formula (IA), L² is —S(O)₂—, X is a substituted or unsubstituted phenyl, R¹ is H, and R⁵ is a substituted or unsubstituted benzimidazolyl.

In another embodiment of the compounds of formula (IA), R¹ and R⁵ are both H, L² is —C(O)—, and X is a substituted or unsubstituted heterocyclyl.

In another embodiment of the compounds of formula (IA), R¹ and R⁵ are both H, L² is —C(O)—, and X is a substituted or unsubstituted piperidinyl.

In another embodiment of the compounds of formula (IA), R¹ and R⁵ are both H, L² is —C(O)—, and X is a substituted or unsubstituted pyrrolidinyl.

In another embodiment of the compounds of formula (IA), R¹ and R⁵ are both H, L² is —C(O)—, and X is a substituted or unsubstituted piperazinyl.

In another embodiment of the compounds of formula (IA), R¹ is H, R⁵ is substituted or unsubstituted arylalkyl, L² is a covalent bond, and X is substituted or unsubstituted cycloalkyl.

In another embodiment of the compounds of formula (IA), R¹ is H, R⁵ is substituted or unsubstituted benzyl, L² is a covalent bond, and X is substituted or unsubstituted cyclohexyl.

In another embodiment of the compounds of formula (IA), R¹ is H, R⁵ is substituted or unsubstituted benzyl, L² is a covalent bond, and X is substituted or unsubstituted tetradehydronaphthalenyl.

In another embodiment of the compounds of formula (IA), R¹ and R⁵ are both H, L² and L³ are both —C(O)—, L⁴ is —C(R⁴)₂—, X is substituted or unsubstituted phenyl, and Y is substituted or unsubstituted phenyl.

In another embodiment of the compounds of formula (IA), R¹ and R⁵ are both H, L² and L³ are both —C(O)—, L⁴ is —C(R⁴)₂—, X is substituted or unsubstituted phenyl, and Y is substituted or unsubstituted pyridyl.

In another embodiment of the compounds of formula (IA), R¹ and R⁵ are both H, L² is —C(O)—, L³ is —C(R⁴)₂—, L⁴ is —C(R⁴)₂—, X is substituted or unsubstituted phenyl, and Y is substituted or unsubstituted phenyl.

In another embodiment of the compounds of formula (IA), R¹ and R⁵ are both H, L² is —C(O)—, L³ is —C(R⁴)₂—, L⁴ is —C(R⁴)₂—, X is substituted or unsubstituted phenyl, and Y is substituted or unsubstituted pyridyl.

In another embodiment of the compounds of formula (IA), R¹ and R⁵ are both H, L² is —S(O)₂—, L³ is —C(O)—, L⁴ is —C(R⁴)₂—, X is substituted or unsubstituted phenyl, and Y is substituted or unsubstituted heteroaryl.

In another embodiment of the compounds of formula (IA), R¹ and R⁵ are both H, L² is —S(O)₂—, L³ and L⁴ are both —C(R⁴)₂—, X is substituted or unsubstituted phenyl, and Y is substituted or unsubstituted heteroaryl.

In another embodiment, the compounds of the present invention, or pharmaceutically acceptable salts, solvates, prodrugs, tautomers, or esters thereof, have the structure of formula (IB):

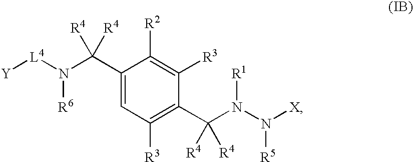

wherein R¹, R², each R³, each R⁴, R⁵, R⁶, L⁴, X and Y are independently as defined herein.

In another embodiment of the compounds of formula (IB),

L⁴ is a covalent bond or —C(R⁴)₂—;
X is selected from the group consisting of substituted or unsubstituted aryl, and substituted or unsubstituted heteroaryl;
Y is selected from the group consisting of substituted or unsubstituted heteroaryl, substituted or unsubstituted aryl, alkoxyalkyl, and hydroxyalkyl;
R¹ is selected from the group consisting of H, alkyl, substituted or unsubstituted arylalkyl, substituted or unsubstituted —S(O)₂-aryl, substituted or unsubstituted heteroarylalkyl, substituted or unsubstituted cycloalkyl, substituted or unsubstituted aryl, and substituted or unsubstituted heteroaryl;
R² and each R³ are each independently selected from the group consisting of H, alkyl, halo, haloalkyl, alkoxy, hydroxy, acyl, substituted or unsubstituted amino, alkyl-S(O)ₘ—, substituted or unsubstituted amino-S(O)ₘ—, substituted or unsubstituted amido, carboxy, alkoxycarbonyl, aminocarbonyl, and —CN, wherein m is an integer ranging from 0-2;
each R⁴ is independently selected from the group consisting of H, alkyl, substituted or unsubstituted aryl, and substituted or unsubstituted heteroaryl;
R⁵ is selected from the group consisting of H, substituted or unsubstituted heteroarylalkyl, alkyl, aminoalkyl, substituted or unsubstituted cycloalkyl, substituted or unsubstituted arylalkyl, substituted or unsubstituted aryl, and substituted or unsubstituted heteroaryl; and
R⁶ is selected from the group consisting of H, aminoalkyl, substituted or unsubstituted arylalkyl, substituted or unsubstituted cycloalkyl, alkoxyalkyl, hydroxyalkyl, substituted or unsubstituted heteroarylalkyl, substituted or unsubstituted aryl, substituted or unsubstituted heteroaryl, acyl, carboxy, alkoxycarbonyl, and substituted or unsubstituted aminocarbonyl; or
R⁶ and -L⁴-Y, together with the nitrogen atom to which they are both attached, form a substituted or unsubstituted heterocyclyl ring.

In another embodiment of the compounds of formula (IB), each of R⁴, R⁵, and R⁶ are H.

In another embodiment of the compounds of formula (IB), R⁴ is H; R¹ is selected from the group consisting of H, substituted or unsubstituted arylalkyl, substituted or unsubstituted heteroarylalkyl, and substituted or unsubstituted —S(O)₂-aryl; X is selected from the group consisting of substituted or unsubstituted aryl, and substituted or unsubstituted heteroaryl; and Y is selected from the group consisting of substituted or unsubstituted aryl, substituted or unsubstituted heteroaryl, alkoxyalkyl, and hydroxyalkyl.

In another embodiment of the compounds of formula (IB), R⁴ is H; R¹ is selected from the group consisting of H, substituted or unsubstituted benzyl, substituted or unsubstituted benzimidazolylalkyl, and substituted or unsubstituted —S(O)₂-phenyl; X is selected from the group consisting of substituted or unsubstituted phenyl, substituted or unsubstituted pyrimidyl, and substituted or unsubstituted benzimidazoyl; and Y is selected from the group consisting of substituted or unsubstituted pyridyl, substituted or unsubstituted pyrimidyl, substituted or unsubstituted phenyl, and methoxyalkyl.

In another embodiment of the compounds of formula (IB), R⁴ is H; R¹ is selected from the group consisting of substituted or unsubstituted arylalkyl, substituted or unsubstituted heteroarylalkyl, and substituted or unsubstituted —S(O)₂-aryl; X is selected from the group consisting of substituted or unsubstituted aryl, and substituted or unsubstituted heteroaryl; and R⁶ and -L⁴-Y, together with the nitrogen atom to which they are both attached, form a substituted or unsubstituted heterocyclyl ring.

In another embodiment of the compounds of formula (IB), R⁶ and -L⁴-Y, together with the nitrogen atom to which they are both attached, form a substituted or unsubstituted morpholinyl ring, or a substituted or unsubstituted 1,7-dioxa-4,10-diazacyclododecanyl ring.

In another embodiment of the compounds of formula (IB), L⁴ is —C(R⁴)₂— and each R⁴ is H.

In another embodiment of the compounds of formula (IB), L⁴ is —C(R⁴)₂— and Y is substituted or unsubstituted heteroaryl.

In another embodiment of the compounds of formula (IB), L⁴ is —C(R⁴)₂— and Y is substituted or unsubstituted pyridyl.

In another embodiment of the compounds of formula (IB), L⁴ is —C(R⁴)₂—, Y is substituted or unsubstituted heteroaryl, and each R⁴ is H.

In another embodiment of the compounds of formula (IB), L⁴ is —C(R⁴)₂— and Y is substituted or unsubstituted pyridyl, and each R⁴ is H.

In another embodiment of the compounds of formula (IB), L⁴ is —C(R⁴)₂— and Y is substituted or unsubstituted 2-pyridyl, and each R⁴ is H.

In another embodiment of the compounds of formula (IB), L⁴ is a covalent bond.

In another embodiment of the compounds of formula (IB), L⁴ is a covalent bond, and Y is substituted or unsubstituted heteroaryl.

In another embodiment of the compounds of formula (IB), L⁴ is a covalent bond, and Y is substituted or unsubstituted pyrimidyl.

In another embodiment of the compounds of formula (IB), R⁶ and -L⁴-Y, together with the nitrogen atom to which they are both attached, form a substituted or unsubstituted heterocyclyl ring.

In another embodiment of the compounds of formula (IB), R⁶ and -L⁴-Y, together with the nitrogen atom to which they are both attached, form a substituted or unsubstituted morpholinyl.

In another embodiment of the compounds of formula (IB), R⁶ and -L⁴-Y, together with the nitrogen atom to which they are both attached, form a substituted or unsubstituted dioxadiazacyclododecanyl.

In another embodiment of the compounds of formula (IB), R⁶ and -L⁴-Y, together with the nitrogen atom to which they are both attached, form a substituted or unsubstituted 1,7-dioxa-4,10-diazacyclododecanyl.

In another embodiment of the compounds of formula (IB), R⁶ and -L⁴-Y, together with the nitrogen atom to which they are both attached, form a substituted or unsubstituted piperidinyl.

In another embodiment of the compounds of formula (IB), R⁶ and -L⁴-Y, together with the nitrogen atom to which they are both attached, form a substituted or unsubstituted piperazinyl.

In another embodiment of the compounds of formula (IB), R⁶ and -L⁴-Y, together with the nitrogen atom to which they are both attached, form a substituted or unsubstituted pyrrolidinyl.

In another embodiment of the compounds of formula (IB), R⁶ is H.

In another embodiment of the compounds of formula (IB), R⁶ is H, and -L⁴-Y is alkoxyalkyl.

In another embodiment of the compounds of formula (IB), R⁶ is H, and -L⁴-Y is CH₃—O—CH₂CH₂—

In another embodiment of the compounds of formula (IB), R⁶ is H, and -L⁴-Y is CH₃CH₂—O—CH₂CH₂—.

In another embodiment of the compounds of formula (IB), R⁶ is H, and -L⁴-Y is CH₃—C—CH₂CH₂CH₂—.

In another embodiment of the compounds of formula (IB), R⁶ is H, and -L⁴-Y is HO—CH₂CH₂—.

In another embodiment of the compounds of formula (IB), R⁶ is H, and -L⁴-Y is HO—CH₂CH₂CH₂—.

In another embodiment of the compounds of formula (IB), R¹ and R⁵ are both H.

In another embodiment of the compounds of formula (IB), R¹ and R⁵ are both H, and X is a substituted or unsubstituted aromatic.

In another embodiment of the compounds of formula (IB), R¹ and R⁵ are both H, and X is a substituted or unsubstituted phenyl.

In another embodiment of the compounds of formula (IB), R¹ and R⁵ are both H, and X is a substituted or unsubstituted heteroaromatic.

In another embodiment of the compounds of formula (IB), R¹ and R⁵ are both H, and X is a substituted or unsubstituted pyrimidyl.

In another embodiment of the compounds of formula (IB), R¹ and R⁵ are both H, and X is a substituted or unsubstituted benzimidazolyl.

In another embodiment of the compounds of formula (IB), R¹ is substituted or unsubstituted arylalkyl.

In another embodiment of the compounds of formula (IB), R¹ is substituted or unsubstituted benzyl.

In another embodiment of the compounds of formula (IB), R¹ is substituted or unsubstituted heteroarylalkyl.

In another embodiment of the compounds of formula (IB), R¹ is substituted or unsubstituted benzimidazolylmethyl.

In another embodiment of the compounds of formula (IB), R¹ is substituted or unsubstituted pyridylmethyl.

In another embodiment of the compounds of formula (IB), R¹ is substituted or unsubstituted —S(O)₂-aryl.

In another embodiment of the compounds of formula (IB), R¹ is substituted or unsubstituted —S(O)₂-phenyl.

In another embodiment of the compounds of formula (IB), R¹ is substituted or unsubstituted —S(O)₂-heteroaryl.

In another embodiment of the compounds of formula (IB), R¹ is substituted or unsubstituted —S(O)₂-pyridyl.

In another embodiment of the compounds of formula (IB), R¹ is substituted or unsubstituted arylalkyl and X is substituted or unsubstituted aryl.

In another embodiment of the compounds of formula (IB), R¹ is substituted or unsubstituted benzyl and X is substituted or unsubstituted phenyl.

In another embodiment of the compounds of formula (IB), R¹ is substituted or unsubstituted arylalkyl and X is substituted or unsubstituted heteroaryl.

In another embodiment of the compounds of formula (IB), R¹ is substituted or unsubstituted benzyl and X is substituted or unsubstituted pyrimidyl.

In another embodiment of the compounds of formula (IB), R¹ is substituted or unsubstituted benzyl and X is substituted or unsubstituted pyridyl.

In another embodiment of the compounds of formula (IB), R¹ is substituted or unsubstituted heteroarylalkyl and X is substituted or unsubstituted heteroaryl.

In another embodiment of the compounds of formula (IB), R¹ is substituted or unsubstituted benzimidazolylalkyl and X is substituted or unsubstituted pyrimidyl.

In another embodiment of the compounds of formula (IB), R¹ is substituted or unsubstituted benzimidazolylalkyl and X is substituted or unsubstituted pyridyl.

In another embodiment of the compounds of formula (IB), R¹ is substituted or unsubstituted pyridylalkyl and X is substituted or unsubstituted pyrimidyl.

In another embodiment of the compounds of formula (IB), R¹ is substituted or unsubstituted benzimidazolylmethyl and X is substituted or unsubstituted pyrimidyl.

In another embodiment of the compounds of formula (IB), R¹ is substituted or unsubstituted benzimidazolylmethyl and X is substituted or unsubstituted pyridyl.

In another embodiment of the compounds of formula (IB), R¹ is substituted or unsubstituted pyridylmethyl and X is substituted or unsubstituted pyrimidyl.

In another embodiment of the compounds of formula (IB), R¹ is substituted or unsubstituted arylalkyl and X is substituted or unsubstituted heteroaryl.

In another embodiment of the compounds of formula (IB), R¹ is substituted or unsubstituted benzyl and X is substituted or unsubstituted pyrimidyl.

In another embodiment of the compounds of formula (IB), R¹ is substituted or unsubstituted benzyl and X is substituted or unsubstituted pyridyl.

In another embodiment of the compounds of formula (IB), R¹ is substituted or unsubstituted benzyl and X is substituted or unsubstituted benzimidazolyl.

In another embodiment of the compounds of formula (IB), R¹ is substituted or unsubstituted heteroarylalkyl and X is substituted or unsubstituted aryl.

In another embodiment of the compounds of formula (IB), R¹ is substituted or unsubstituted pyridylmethyl and X is substituted or unsubstituted phenyl.

In another embodiment of the compounds of formula (IB), R¹ is substituted or unsubstituted benzimidazolylmethyl and X is substituted or unsubstituted phenyl.

In another embodiment of the compounds of formula (IB), R¹ is substituted or unsubstituted pyrimidylmethyl and X is substituted or unsubstituted phenyl.

In another embodiment of the compounds of formula (IB), R¹ is substituted or unsubstituted —S(O)₂-aryl and X is substituted or unsubstituted aryl.

In another embodiment of the compounds of formula (IB), R¹ is substituted or unsubstituted —S(O)₂-phenyl and X is substituted or unsubstituted phenyl.

In another embodiment of the compounds of formula (IB), R¹ is substituted or unsubstituted —S(O)₂-heteroaryl and X is substituted or unsubstituted heteroaryl.

In another embodiment of the compounds of formula (IB), R¹ is substituted or unsubstituted —S(O)₂-pyridyl and X is substituted or unsubstituted pyrimidyl.

In another embodiment of the compounds of formula (IB), R¹ is substituted or unsubstituted —S(O)₂-pyridyl and X is substituted or unsubstituted benzimidazolyl.

In another embodiment of the compounds of formula (IB), R¹ is substituted or unsubstituted —S(O)₂-pyridyl and X is substituted or unsubstituted pyridyl.

In another embodiment of the compounds of formula (IB), R¹ is substituted or unsubstituted —S(O)₂-benzimidazolyl and X is substituted or unsubstituted pyrimidyl.

In another embodiment of the compounds of formula (IB), R¹ is substituted or unsubstituted —S(O)₂-benzimidazolyl and X is substituted or unsubstituted benzimidazolyl.

In another embodiment of the compounds of formula (IB), R¹ is substituted or unsubstituted —S(O)₂-benzimidazolyl and X is substituted or unsubstituted pyridyl.

In another embodiment of the compounds of formula (IB), R¹ is substituted or unsubstituted —S(O)₂-pyrimidyl and X is substituted or unsubstituted pyrimidyl.

In another embodiment of the compounds of formula (IB), R¹ is substituted or unsubstituted —S(O)₂-pyrimidyl and X is substituted or unsubstituted benzimidazolyl.

In another embodiment of the compounds of formula (IB), R¹ is substituted or unsubstituted —S(O)₂-pyrimidyl and X is substituted or unsubstituted pyridyl.

In another embodiment of the compounds of formula (IB), R¹ is substituted or unsubstituted —S(O)₂-heteroaryl and X is substituted or unsubstituted aryl.

In another embodiment of the compounds of formula (IB), R¹ is substituted or unsubstituted —S(O)₂-pyridyl and X is substituted or unsubstituted phenyl.

In another embodiment of the compounds of formula (IB), R¹ is substituted or unsubstituted —S(O)₂-pyrimidyl and X is substituted or unsubstituted phenyl.

In another embodiment of the compounds of formula (IB), R¹ is substituted or unsubstituted —S(O)₂-benzimidazolyl and X is substituted or unsubstituted phenyl.

In another embodiment of the compounds of formula (IB), R¹ is substituted or unsubstituted —S(O)₂-aryl and X is substituted or unsubstituted heteroaryl.

In another embodiment of the compounds of formula (IB), R¹ is substituted or unsubstituted —S(O)₂-phenyl and X is substituted or unsubstituted pyridyl.

In another embodiment of the compounds of formula (IB), R¹ is substituted or unsubstituted —S(O)₂-phenyl and X is substituted or unsubstituted pyrimidyl.

In another embodiment of the compounds of formula (IB), R¹ is substituted or unsubstituted —S(O)₂-phenyl and X is substituted or unsubstituted benzimidazolyl.

In another embodiment, the compounds of the present invention, or pharmaceutically acceptable salts, solvates, prodrugs, tautomers, or esters thereof, have the structure of formula (IC):

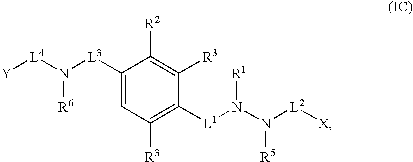

wherein R¹, R², each R³, R⁵, R⁶, L¹, L², L³, L⁴, X and Y are independently as defined herein.

In another embodiment of the compounds of formula (IC), L¹ is selected from the group consisting of a covalent bond, —C(R⁴)₂—, —C(O)—, —S(O)₂—, —S(O)—, and —C(R⁴)═; L² is selected from the group consisting of a covalent bond, —S(O)₂—, —S(O)—, —C(O)—, —C(O)O—, and —C(R⁴)₂—; L³ is selected from the group consisting of —C(O)—, —C(R⁴)₂—, —S(O)₂—, and —S(O)—; L⁴ is selected from the group consisting of a covalent bond, —C(R⁴)₂—, and —C(O)—; X is selected from the group consisting of H, alkyl, substituted or unsubstituted aryl, substituted or unsubstituted arylalkyl, substituted or unsubstituted heteroaryl, substituted or unsubstituted amino, and substituted or unsubstituted aminoalkyl; Y is selected from the group consisting of H, substituted or unsubstituted heteroaryl, substituted or unsubstituted aryl, hydroxyalkyl, alkoxyalkyl, substituted or unsubstituted —C(O)-heterocyclyl, and alkyl; R¹ is selected from the group consisting of H, alkyl, substituted or unsubstituted arylalkyl, and substituted or unsubstituted heteroarylalkyl; R² and each R³ are each independently selected from the group consisting of H, alkyl, halo, haloalkyl, alkoxy, hydroxy, acyl, substituted or unsubstituted amino, alkyl-S(O)ₘ—, substituted or unsubstituted amino-S(O)ₘ—, substituted or unsubstituted amido, carboxy, alkoxycarbonyl, aminocarbonyl, and —CN, wherein m is an integer ranging from 0-2; each R⁴ is independently selected from the group consisting of H, alkyl, alkylthioalkyl, and thioalkyl; R⁵ is selected from the group consisting of H, substituted or unsubstituted heteroarylalkyl, alkyl, alkyl-S(O)₂-substituted or unsubstituted aminoalkyl, substituted or unsubstituted cycloalkyl, and substituted or unsubstituted arylalkyl; or R⁵ and -L²-X, together with the nitrogen atom to which they are both attached, form a substituted or unsubstituted heterocyclyl ring; or R⁵ and R¹, together with the nitrogens to which they are each attached, form a substituted or unsubstituted heterocyclyl ring; and R⁶ is selected from the group consisting of H, substituted or unsubstituted cycloalkyl, substituted or unsubstituted arylalkyl, alkoxyalkyl, hydroxyalkyl, and substituted or unsubstituted heteroarylalkyl; or R⁶ and -L⁴-Y, together with the nitrogen atom to which they are both attached, form a substituted or unsubstituted heterocyclyl ring.

In another embodiment of the compounds of formula (IC), L¹ is —C(O)—; L² is a covalent bond; L³ is —CH₂—; L⁴ is a covalent bond or —C(R⁴)₂—; X is selected from the group consisting of substituted or unsubstituted aryl, and substituted or unsubstituted heteroaryl; each R⁴ is independently selected from the group consisting of H, alkyl, alkylthioalkyl, and thioalkyl; and R⁶ is selected from the group consisting of H, substituted or unsubstituted arylalkyl, and substituted or unsubstituted heteroarylalkyl; or R⁶ and -L⁴-Y, together with the nitrogen atom to which they are both attached, form a substituted or unsubstituted heterocyclyl ring.

In another embodiment of the compounds of formula (IC), R¹ and R⁵ are both H.

In another embodiment of the compounds of formula (IC), R¹ and R⁵ are both H; X is selected from the group consisting of substituted or unsubstituted phenyl, and substituted or unsubstituted pyrimidyl, substituted or unsubstituted benzimidazolyl, and pyrrolopyrimidinyl; Y is selected from the group consisting of substituted or unsubstituted pyrimidyl, substituted or unsubstituted pyridyl, substituted or unsubstituted phenyl, methoxyalkyl, methyl, substituted or unsubstituted —C(O)-morpholinyl, and substituted or unsubstituted piperazinyl; and R⁶ is H or methoxyethyl.

In another embodiment of the compounds of formula (IC), R¹ and R⁵ are both H; and R⁶ and -L⁴-Y, together with the nitrogen atom to which they are both attached, form a substituted or unsubstituted 1,7-dioxa-4,10-diazacyclododecanyl ring, a substituted or unsubstituted piperazinyl ring, and a substituted or unsubstituted tetraazacyclotetradecanyl ring.

In another embodiment of the compounds of formula (IC), L¹ is —C(O)—; R⁵ and -L²-X, together with the nitrogen atom to which they are both attached, form a substituted or unsubstituted heterocyclyl ring; L⁴ is —C(R⁴)₂— or —C(O)—; Y is selected from the group consisting of substituted or unsubstituted heteroaryl, and substituted or unsubstituted aryl; R¹ is selected from the group consisting of H, alkyl, substituted or unsubstituted arylalkyl, and substituted or unsubstituted heteroarylalkyl; each R⁴ is independently H or alkyl; and R⁶ is selected from the group consisting of H, substituted or unsubstituted arylalkyl, and substituted or unsubstituted heteroarylalkyl.

In another embodiment of the compounds of formula (IC), L¹ is —C(O)—; R⁵ and -L²-X, together with the nitrogen atom to which they are both attached, form a substituted or unsubstituted heterocyclyl ring; L⁴ is —C(R⁴)₂— or —C(O)—; Y is selected from the group consisting of substituted or unsubstituted heteroaryl, and substituted or unsubstituted aryl; R¹ is selected from the group consisting of H, substituted or unsubstituted benzimidazolylalkyl, and substituted or unsubstituted benzyl; R⁴ and R⁶ are H; R⁵ and -L²-X, together with the nitrogen atom to which they are both attached, form a substituted or unsubstituted morpholinyl ring; and Y is selected from the group consisting of substituted or unsubstituted thienyl, and substituted or unsubstituted phenyl.

In another embodiment of the compounds of formula (IC), L¹ is —S(O)₂—; L² is a covalent bond; L³ is —C(R⁴)₂—; L⁴ is a covalent bond or —C(R⁴)₂—; X is selected from the group consisting of substituted or unsubstituted aryl, and substituted or unsubstituted heteroaryl; Y is selected from the group consisting of substituted or unsubstituted heteroaryl, substituted or unsubstituted aryl, hydroxyalkyl, and alkoxyalkyl; R¹ is selected from the group consisting of H, alkyl, substituted or unsubstituted arylalkyl, and substituted or unsubstituted heteroarylalkyl; each R⁴ is independently H or alkyl; and R⁵ is selected from the group consisting of H, substituted or unsubstituted heteroarylalkyl, alkyl, and substituted or unsubstituted arylalkyl.

In another embodiment of the compounds of formula (IC), L¹ and L³ are both —C(O)—; L² is a covalent bond; L⁴ is —C(R⁴)₂—; and X and Y are independently selected from the group consisting of substituted or unsubstituted aryl, and substituted or unsubstituted heteroaryl.

In another embodiment of the compounds of formula (IC), L¹ is —C(O)—; L² is —C(R⁴)₂—, —S(O)₂—, or —C(O)—; L³ is —C(R⁴)₂—; L⁴ is —C(R⁴)₂—; or R⁶ and -L⁴-Y, together with the nitrogen atom to which they are both attached, form a substituted or unsubstituted heterocyclyl ring; X is selected from the group consisting of substituted or unsubstituted aryl, substituted or unsubstituted heteroaryl, and amino; Y is selected from the group consisting of substituted or unsubstituted heteroaryl, and substituted or unsubstituted aryl; each R⁴ is independently H or alkyl; and at least one of R¹ and R⁵ is not H.

In another embodiment of the compounds of formula (IC), L³ is —C(R⁴)₂—.

In another embodiment of the compounds of formula (IC), L³ is —CH₂—.

In another embodiment of the compounds of formula (IC), L³ is —C(R⁴)₂— and L⁴ is —C(R⁴)₂—.

In another embodiment of the compounds of formula (IC), L³ is —CH₂— and L⁴ is —CH₂—.

In another embodiment of the compounds of formula (IC), L³ is —CH₂— and L⁴ is —CH(alkylthioalkyl)-.

In another embodiment of the compounds of formula (IC), L³ is —CH₂— and L⁴ is —CH(CH₂SCH₃)—.

In another embodiment of the compounds of formula (IC), L³ is —CH₂— and L⁴ is —CH(CH₂CH₂SCH₃)—.

In another embodiment of the compounds of formula (IC), L³ is —CH₂— and L⁴ is —CH(CH₂SCH₂CH₃)—.

In another embodiment of the compounds of formula (IC), L³ is —CH₂— and L⁴ is —CH(CH₂CH₂SCH₂CH₃)—.

In another embodiment of the compounds of formula (IC), L³ is —C(R⁴)₂— and L⁴ is a covalent bond.

In another embodiment of the compounds of formula (IC), L³ is —C(R⁴)₂— and L⁴ is —C(O)—.

In another embodiment of the compounds of formula (IC), L³ and L⁴ are both —C(R⁴)₂—, and Y is substituted or unsubstituted aryl.

In another embodiment of the compounds of formula (IC), L³ and L⁴ are both —C(R⁴)₂—, and Y is substituted or unsubstituted phenyl.

In another embodiment of the compounds of formula (IC), L³ and L⁴ are both —CH₂—, and Y is substituted or unsubstituted phenyl.

In another embodiment of the compounds of formula (IC), L³ and L⁴ are both —C(R⁴)₂—, and Y is substituted or unsubstituted heteroaryl.

In another embodiment of the compounds of formula (IC), L³ and L⁴ are both —C(R⁴)₂—, and Y is substituted or unsubstituted pyridyl.

In another embodiment of the compounds of formula (IC), L³ and L⁴ are both —C(R⁴)₂—, and Y is substituted or unsubstituted thienyl.

In another embodiment of the compounds of formula (IC), L³ and L⁴ are both —C(R⁴)₂—, and Y is substituted or unsubstituted pyrimidyl.

In another embodiment of the compounds of formula (IC), L³ and L⁴ are both —C(R⁴)₂—, and Y is substituted or unsubstituted indazolyl.

In another embodiment of the compounds of formula (IC), L³ and L⁴ are both —C(R⁴)₂—, and Y is substituted or unsubstituted purinyl.

In another embodiment of the compounds of formula (IC), L³ and L⁴ are both —CH₂—, and Y is substituted or unsubstituted heteroaryl.

In another embodiment of the compounds of formula (IC), L³ and L⁴ are both —CH₂—, and Y is substituted or unsubstituted pyridyl.

In another embodiment of the compounds of formula (IC), L³ and L⁴ are both —CH₂—, and Y is substituted or unsubstituted thienyl.

In another embodiment of the compounds of formula (IC), L³ and L⁴ are both —CH₂—, and Y is substituted or unsubstituted pyrimidyl.

In another embodiment of the compounds of formula (IC), L³ and L⁴ are both —CH₂—, and Y is substituted or unsubstituted indazolyl.

In another embodiment of the compounds of formula (IC), L³ and L⁴ are both —CH₂—, and Y is substituted or unsubstituted purinyl.

In another embodiment of the compounds of formula (IC), L³ and L⁴ are both —C(R⁴)₂—, and Y is alkoxyalkyl.

In another embodiment of the compounds of formula (IC), L³ and L⁴ are both —CH₂—, and Y is alkoxyalkyl.

In another embodiment of the compounds of formula (IC), L³ and L⁴ are both —CH₂—, and Y is CH₃—O—CH₂—.

In another embodiment of the compounds of formula (IC), L³ and L⁴ are both —CH₂—, and Y is CH₃—O—CH₂CH₂—.

In another embodiment of the compounds of formula (IC), L³ and L⁴ are both —CH₂—, and Y is CH₃CH₂—O—CH₂—.

In another embodiment of the compounds of formula (IC), L³ and L⁴ are both —CH₂—, and Y is CH₃CH₂—O—CH₂CH₂—.

In another embodiment of the compounds of formula (IC), L³ and L⁴ are both —C(R⁴)₂—, and Y is substituted or unsubstituted —C(O)-heterocyclyl.

In another embodiment of the compounds of formula (IC), L³ and L⁴ are both —C(R⁴)₂—, and Y is substituted or unsubstituted —C(O)-pyrrolyl.

In another embodiment of the compounds of formula (IC), L³ and L⁴ are both —C(R⁴)₂—, and Y is substituted or unsubstituted —C(O)-piperidinyl.

In another embodiment of the compounds of formula (IC), L³ and L⁴ are both —C(R⁴)₂—, and Y is substituted or unsubstituted —C(O)-piperazinyl.

In another embodiment of the compounds of formula (IC), L³ and L⁴ are both —C(R⁴)₂—, and Y is substituted or unsubstituted —C(O)-morpholinyl.

In another embodiment of the compounds of formula (IC), L³ and L⁴ are both —C(R⁴)₂—, and Y is substituted or unsubstituted —C(O)-furanyl.

In another embodiment of the compounds of formula (IC), L³ and L⁴ are both —C(R⁴)₂—, and Y is substituted or unsubstituted —C(O)-thienyl.

In another embodiment of the compounds of formula (IC), L³ and L⁴ are both —CH₂—, and Y is substituted or unsubstituted —C(O)-heterocyclyl.

In another embodiment of the compounds of formula (IC), L³ and L⁴ are both —CH₂—, and Y is substituted or unsubstituted —C(O)-pyrrolyl.

In another embodiment of the compounds of formula (IC), L³ and L⁴ are both —CH₂—, and Y is substituted or unsubstituted —C(O)-piperidinyl.

In another embodiment of the compounds of formula (IC), L³ and L⁴ are both —CH₂—, and Y is substituted or unsubstituted —C(O)-piperazinyl.

In another embodiment of the compounds of formula (IC), L³ and L⁴ are both —CH₂—, and Y is substituted or unsubstituted —C(O)-morpholinyl.

In another embodiment of the compounds of formula (IC), L³ and L⁴ are both —CH₂—, and Y is substituted or unsubstituted —C(O)-furanyl.

In another embodiment of the compounds of formula (IC), L³ and L⁴ are both —CH₂—, and Y is substituted or unsubstituted —C(O)-thienyl.

In another embodiment of the compounds of formula (IC), R⁶ and -L⁴-Y, together with the nitrogen atom to which they are both attached, form a substituted or unsubstituted heterocyclyl ring.

In another embodiment of the compounds of formula (IC), R⁶ and -L⁴-Y, together with the nitrogen atom to which they are both attached, form a substituted or unsubstituted piperidinyl ring.

In another embodiment of the compounds of formula (IC), R⁶ and -L⁴-Y, together with the nitrogen atom to which they are both attached, form a substituted or unsubstituted piperazinyl ring.

In another embodiment of the compounds of formula (IC), R⁶ and -L⁴-Y, together with the nitrogen atom to which they are both attached, form a substituted or unsubstituted pyrrolidinyl ring.

In another embodiment of the compounds of formula (IC), L³ is —C(R⁴)₂—, and R⁶ and -L⁴-Y, together with the nitrogen atom to which they are both attached, form a substituted or unsubstituted heterocyclyl ring.

In another embodiment of the compounds of formula (IC), L³ is —C(R⁴)₂—, and R⁶ and -L⁴-Y, together with the nitrogen atom to which they are both attached, form a substituted or unsubstituted piperidinyl ring.

In another embodiment of the compounds of formula (IC), L³ is —C(R⁴)₂—, and R⁶ and -L⁴-Y, together with the nitrogen atom to which they are both attached, form a substituted or unsubstituted piperazinyl ring.

In another embodiment of the compounds of formula (IC), L³ is —C(R⁴)₂—, and R⁶ and -L⁴-Y, together with the nitrogen atom to which they are both attached, form a substituted or unsubstituted pyrrolidinyl ring.

In another embodiment of the compounds of formula (IC), L³ is —C(R⁴)₂—, and R⁶ and -L⁴-Y, together with the nitrogen atom to which they are both attached, form a substituted or unsubstituted dioxadiazacyclododecanyl ring.

In another embodiment of the compounds of formula (IC), L³ is —C(R⁴)₂—, and R⁶ and -L⁴-Y, together with the nitrogen atom to which they are both attached, form a substituted or unsubstituted tetraazacyclotetradecanyl ring.

In another embodiment of the compounds of formula (IC), L³ is —C(R⁴)₂—, and R⁶ and -L⁴-Y, together with the nitrogen atom to which they are both attached, form a substituted or unsubstituted furanyl ring.

In another embodiment of the compounds of formula (IC), L³ is —CH₂—, and R⁶ and -L⁴-Y, together with the nitrogen atom to which they are both attached, form a substituted or unsubstituted heterocyclyl ring.

In another embodiment of the compounds of formula (IC), L³ is —CH₂—, and R⁶ and -L⁴-Y, together with the nitrogen atom to which they are both attached, form a substituted or unsubstituted piperidinyl ring.

In another embodiment of the compounds of formula (IC), L³ is —CH₂—, and R⁶ and -L⁴-Y, together with the nitrogen atom to which they are both attached, form a substituted or unsubstituted piperazinyl ring.

In another embodiment of the compounds of formula (IC), L³ is —CH₂—, and R⁶ and -L⁴-Y, together with the nitrogen atom to which they are both attached, form a substituted or unsubstituted pyrrolidinyl ring.

In another embodiment of the compounds of formula (IC), L³ is —CH₂—, and R⁶ and -L⁴-Y, together with the nitrogen atom to which they are both attached, form a substituted or unsubstituted dioxadiazacyclododecanyl ring.

In another embodiment of the compounds of formula (IC), L³ is —CH₂—, and R⁶ and -L⁴-Y, together with the nitrogen atom to which they are both attached, form a substituted or unsubstituted tetraazacyclotetradecanyl ring.

In another embodiment of the compounds of formula (IC), L³ is —CH₂—, and R⁶ and -L⁴-Y, together with the nitrogen atom to which they are both attached, form a substituted or unsubstituted furanyl ring.

In another embodiment of the compounds of formula (IC), L³ is —C(O)—.

In another embodiment of the compounds of formula (IC), L³ is —C(O)— and L⁴ is —C(R⁴)₂—.

In another embodiment of the compounds of formula (IC), L³ is —C(O)—, L⁴ is —C(R⁴)₂—, and Y is substituted or unsubstituted aryl.

In another embodiment of the compounds of formula (IC), L³ is —C(O)—, L⁴ is —C(R⁴)₂—, and Y is substituted or unsubstituted heteroaryl.

In another embodiment of the compounds of formula (IC), L³ is —C(O)—, L⁴ is —C(R⁴)₂—, and Y is substituted or unsubstituted phenyl.

In another embodiment of the compounds of formula (IC), L³ is —C(O)—, L⁴ is —C(R⁴)₂—, and Y is substituted or unsubstituted pyridyl.

In another embodiment of the compounds of formula (IC), L³ is —C(O)—, L⁴ is —C(R⁴)₂—, and Y is substituted or unsubstituted 2-pyridyl.

In another embodiment of the compounds of formula (IC), L³ is —C(O)—, L⁴ is —C(R⁴)₂—, and Y is substituted or unsubstituted pyrimidyl.

In another embodiment of the compounds of formula (IC), L³ is —C(O)—, L⁴ is —C(R⁴)₂—, and Y is substituted or unsubstituted benzimidazolyl.

In another embodiment of the compounds of formula (IC), L³ is —C(O)—, L⁴ is —C(R⁴)₂—, and Y is substituted or unsubstituted thienyl.

In another embodiment of the compounds of formula (IC), L³ is —C(O)—, L⁴ is —C(R⁴)₂—, and Y is substituted or unsubstituted quinolinyl.

In another embodiment of the compounds of formula (IC), L³ is —C(O)—, L⁴ is —CH₂—, and Y is substituted or unsubstituted phenyl.

In another embodiment of the compounds of formula (IC), L³ is —C(O)—, L⁴ is —CH₂—, and Y is substituted or unsubstituted pyridyl.

In another embodiment of the compounds of formula (IC), L³ is —C(O)—, L⁴ is —CH₂—, and Y is substituted or unsubstituted 2-pyridyl.

In another embodiment of the compounds of formula (IC), L³ is —C(O)—, L⁴ is —CH₂—, and Y is substituted or unsubstituted pyrimidyl.

In another embodiment of the compounds of formula (IC), L³ is —C(O)—, L⁴ is —CH₂—, and Y is substituted or unsubstituted benzimidazolyl.

In another embodiment of the compounds of formula (IC), L³ is —C(O)—, L⁴ is —CH₂—, and Y is substituted or unsubstituted thienyl.

In another embodiment of the compounds of formula (IC), L³ is —C(O)—, L⁴ is —CH₂—, and Y is substituted or unsubstituted quinolinyl.

In another embodiment of the compounds of formula (IC), L¹ is —C(O)—.

In another embodiment of the compounds of formula (IC), L¹ is —S(O)₂—

In another embodiment of the compounds of formula (IC), L¹ is —S(O)—.

In another embodiment of the compounds of formula (IC), L¹ is —C(O)—, and L² is a covalent bond.

In another embodiment of the compounds of formula (IC), L¹ is —C(O)—, and L² is —C(R⁴)₂—.

In another embodiment of the compounds of formula (IC), L¹ is —C(O)—, and L² is —S(O)₂—.

In another embodiment of the compounds of formula (IC), L¹ is —C(O)—, and L² is —S(O)—.

In another embodiment of the compounds of formula (IC), L¹ is —C(O)—, and L² is —C(O)—.

In another embodiment of the compounds of formula (IC), L¹ is —S(O)₂—, and L² is a covalent bond.

In another embodiment of the compounds of formula (IC), L¹ is —S(O)₂—, and L² is —C(R⁴)₂—.

In another embodiment of the compounds of formula (IC), L¹ is —S(O)₂—, and L² is —S(O)₂—.

In another embodiment of the compounds of formula (IC), L¹ is —S(O)₂—, and L² is —S(O)—.

In another embodiment of the compounds of formula (IC), L¹ is —S(O)₂—, and L² is —C(O)—.

In another embodiment of the compounds of formula (IC), L¹ is —S(O)—, and L² is a covalent bond.

In another embodiment of the compounds of formula (IC), L¹ is —S(O)—, and L² is —C(R⁴)₂—.

In another embodiment of the compounds of formula (IC), L¹ is —S(O)—, and L² is —S(O)₂—.

In another embodiment of the compounds of formula (IC), L¹ is —S(O)—, and L² is —S(O)—.

In another embodiment of the compounds of formula (IC), L¹ is —S(O)—, and L² is —C(O)—.

In another embodiment of the compounds of formula (IC), L¹ is —S(O)₂—, and L² is a covalent bond.

In another embodiment of the compounds of formula (IC), L¹ is —S(O)₂—, and L² is —C(R⁴)₂—.

In another embodiment of the compounds of formula (IC), L¹ is —S(O)₂—, and L² is —S(O)₂—.

In another embodiment of the compounds of formula (IC), L¹ is —S(O)₂—, and L² is —S(O)—.

In another embodiment of the compounds of formula (IC), L¹ is —S(O)₂—, and L² is —C(O)—.

In another embodiment of the compounds of formula (IC), L¹ is —C(O)—, L² is a covalent bond, and X is a substituted or unsubstituted aryl.

In another embodiment of the compounds of formula (IC), L¹ is —C(O)—, L² is —C(R⁴)₂—, and X is a substituted or unsubstituted aryl.

In another embodiment of the compounds of formula (IC), L¹ is —C(O)—, L² is —S(O)₂—, and X is a substituted or unsubstituted aryl.

In another embodiment of the compounds of formula (IC), L¹ is —C(O)—, L² is —S(O)—, and X is a substituted or unsubstituted aryl.

In another embodiment of the compounds of formula (IC), L¹ is —C(O)—, L² is —C(O)—, and X is a substituted or unsubstituted aryl.

In another embodiment of the compounds of formula (IC), L¹ is —S(O)₂—, L² is a covalent bond, and X is a substituted or unsubstituted aryl.

In another embodiment of the compounds of formula (IC), L¹ is —S(O)₂—, L² is —C(R⁴)₂—, and X is a substituted or unsubstituted aryl.

In another embodiment of the compounds of formula (IC), L¹ is —S(O)₂—, L² is —S(O)₂—, and X is a substituted or unsubstituted aryl.

In another embodiment of the compounds of formula (IC), L¹ is —S(O)₂—, L² is —S(O)—, and X is a substituted or unsubstituted aryl.

In another embodiment of the compounds of formula (IC), L¹ is —S(O)₂—, L² is —C(O)—, and X is a substituted or unsubstituted aryl.

In another embodiment of the compounds of formula (IC), L¹ is —S(O)—, L² is a covalent bond, and X is a substituted or unsubstituted aryl.

In another embodiment of the compounds of formula (IC), L¹ is —S(O)—, L² is —C(R⁴)₂—, and X is a substituted or unsubstituted aryl.

In another embodiment of the compounds of formula (IC), L¹ is —S(O)—, L² is —S(O)₂—, and X is a substituted or unsubstituted aryl.

In another embodiment of the compounds of formula (IC), L¹ is —S(O)—, L² is —S(O)—, and X is a substituted or unsubstituted aryl.

In another embodiment of the compounds of formula (IC), L¹ is —S(O)—, L² is —C(O)—, and X is a substituted or unsubstituted aryl.

In another embodiment of the compounds of formula (IC), L¹ is —S(O)₂—, L² is a covalent bond, and X is a substituted or unsubstituted aryl.

In another embodiment of the compounds of formula (IC), L¹ is —S(O)₂—, L² is —C(R⁴)₂—, and X is a substituted or unsubstituted aryl.

In another embodiment of the compounds of formula (IC), L¹ is —S(O)₂—, L² is —S(O)₂—, and X is a substituted or unsubstituted aryl.

In another embodiment of the compounds of formula (IC), L¹ is —S(O)₂—, L² is —S(O)—, and X is a substituted or unsubstituted aryl.

In another embodiment of the compounds of formula (IC), L¹ is —S(O)₂—, L² is —C(O)—, and X is a substituted or unsubstituted aryl.

In another embodiment of the compounds of formula (IC), L¹ is —C(O)—, L² is —CH₂—, and X is a substituted or unsubstituted aryl.

In another embodiment of the compounds of formula (IC), L¹ is —S(O)₂—, L² is —CH₂—, and X is a substituted or unsubstituted aryl.

In another embodiment of the compounds of formula (IC), L¹ is —S(O)—, L² is —CH₂—, and X is a substituted or unsubstituted aryl.

In another embodiment of the compounds of formula (IC), L¹ is —S(O)₂—, L² is —CH₂—, and X is a substituted or unsubstituted aryl.

In another embodiment of the compounds of formula (IC), L¹ is —C(O)—, L² is a covalent bond, and X is a substituted or unsubstituted phenyl.

In another embodiment of the compounds of formula (IC), L¹ is —C(O)—, L² is —C(R⁴)₂—, and X is a substituted or unsubstituted phenyl.

In another embodiment of the compounds of formula (IC), L¹ is —C(O)—, L² is —S(O)₂—, and X is a substituted or unsubstituted phenyl.

In another embodiment of the compounds of formula (IC), L¹ is —C(O)—, L² is —S(O)—, and X is a substituted or unsubstituted phenyl.

In another embodiment of the compounds of formula (IC), L¹ is —C(O)—, L² is —C(O)—, and X is a substituted or unsubstituted phenyl.

In another embodiment of the compounds of formula (IC), L¹ is —S(O)₂—, L² is a covalent bond, and X is a substituted or unsubstituted phenyl.

In another embodiment of the compounds of formula (IC), L¹ is —S(O)₂—, L² is —C(R⁴)₂—, and X is a substituted or unsubstituted phenyl.

In another embodiment of the compounds of formula (IC), L¹ is —S(O)₂—, L² is —S(O)₂—, and X is a substituted or unsubstituted phenyl.

In another embodiment of the compounds of formula (IC), L¹ is —S(O)₂—, L² is —S(O)—, and X is a substituted or unsubstituted phenyl.

In another embodiment of the compounds of formula (IC), L¹ is —S(O)₂—, L² is —C(O)—, and X is a substituted or unsubstituted phenyl.

In another embodiment of the compounds of formula (IC), L¹ is —S(O)—, L² is a covalent bond, and X is a substituted or unsubstituted phenyl.

In another embodiment of the compounds of formula (IC), L¹ is —S(O)—, L² is —C(R⁴)₂—, and X is a substituted or unsubstituted phenyl.

In another embodiment of the compounds of formula (IC), L¹ is —S(O)—, L² is —S(O)₂—, and X is a substituted or unsubstituted phenyl.

In another embodiment of the compounds of formula (IC), L¹ is —S(O)—, L² is —S(O)—, and X is a substituted or unsubstituted phenyl.

In another embodiment of the compounds of formula (IC), L¹ is —S(O)—, L² is —C(O)—, and X is a substituted or unsubstituted phenyl.

In another embodiment of the compounds of formula (IC), L¹ is —S(O)₂—, L² is a covalent bond, and X is a substituted or unsubstituted phenyl.

In another embodiment of the compounds of formula (IC), L¹ is —S(O)₂—, L² is —C(R⁴)₂—, and X is a substituted or unsubstituted phenyl.

In another embodiment of the compounds of formula (IC), L¹ is —S(O)₂—, L² is —S(O)₂—, and X is a substituted or unsubstituted phenyl.

In another embodiment of the compounds of formula (IC), L¹ is —S(O)₂—, L² is —S(O)—, and X is a substituted or unsubstituted phenyl.

In another embodiment of the compounds of formula (IC), L¹ is —S(O)₂—, L² is —C(O)—, and X is a substituted or unsubstituted phenyl.

In another embodiment of the compounds of formula (IC), L¹ is —C(O)—, L² is —CH₂—, and X is a substituted or unsubstituted phenyl.

In another embodiment of the compounds of formula (IC), L¹ is —S(O)₂—, L² is —CH₂—, and X is a substituted or unsubstituted phenyl.

In another embodiment of the compounds of formula (IC), L¹ is —S(O)—, L² is —CH₂—, and X is a substituted or unsubstituted phenyl.

In another embodiment of the compounds of formula (IC), L¹ is —S(O)₂—, L² is —CH₂—, and X is a substituted or unsubstituted phenyl.

In another embodiment of the compounds of formula (IC), L¹ is —C(O)—, L² is a covalent bond, and X is a substituted or unsubstituted heteroaryl.

In another embodiment of the compounds of formula (IC), L¹ is —C(O)—, L² is a covalent bond, and X is a substituted or unsubstituted pyridyl.

In another embodiment of the compounds of formula (IC), L¹ is —C(O)—, L² is a covalent bond, and X is a substituted or unsubstituted pyrimidyl.

In another embodiment of the compounds of formula (IC), L¹ is —C(O)—, L² is a covalent bond, and X is a substituted or unsubstituted benzimidazolyl.

In another embodiment of the compounds of formula (IC), L¹ is —C(O)—, L² is a covalent bond, and X is a substituted or unsubstituted quinolyl.

In another embodiment of the compounds of formula (IC), L¹ is —C(O)—, L² is a covalent bond, and X is a substituted or unsubstituted purinyl.

In another embodiment of the compounds of formula (IC), L¹ is —C(O)—, L² is a covalent bond, and X is a substituted or unsubstituted thienyl.

In another embodiment of the compounds of formula (IC), L¹ is —C(O)—, L² is —C(R⁴)₂—, and X is a substituted or unsubstituted heteroaryl.

In another embodiment of the compounds of formula (IC), L¹ is —C(O)—, L² is —C(R⁴)₂—, and X is a substituted or unsubstituted pyridyl.

In another embodiment of the compounds of formula (IC), L¹ is —C(O)—, L² is —C(R⁴)₂—, and X is a substituted or unsubstituted pyrimidyl.

In another embodiment of the compounds of formula (IC), L¹ is —C(O)—, L² is —C(R⁴)₂—, and X is a substituted or unsubstituted benzimidazolyl.

In another embodiment of the compounds of formula (IC), L¹ is —C(O)—, L² is —C(R⁴)₂—, and X is a substituted or unsubstituted quinolyl.

In another embodiment of the compounds of formula (IC), L¹ is —C(O)—, L² is —C(R⁴)₂—, and X is a substituted or unsubstituted purinyl.

In another embodiment of the compounds of formula (IC), L¹ is —C(O)—, L² is —C(R⁴)₂—, and X is a substituted or unsubstituted thienyl.

In another embodiment of the compounds of formula (IC), L¹ is —C(O)—, L² is —S(O)₂—, and X is a substituted or unsubstituted heteroaryl.

In another embodiment of the compounds of formula (IC), L¹ is —C(O)—, L² is —S(O)₂—, and X is a substituted or unsubstituted pyridyl.

In another embodiment of the compounds of formula (IC), L¹ is —C(O)—, L² is —S(O)₂—, and X is a substituted or unsubstituted pyrimidyl.

In another embodiment of the compounds of formula (IC), L¹ is —C(O)—, L² is —S(O)₂—, and X is a substituted or unsubstituted benzimidazolyl.

In another embodiment of the compounds of formula (IC), L¹ is —C(O)—, L² is —S(O)₂—, and X is a substituted or unsubstituted quinolyl.

In another embodiment of the compounds of formula (IC), L¹ is —C(O)—, L² is —S(O)₂—, and X is a substituted or unsubstituted purinyl.

In another embodiment of the compounds of formula (IC), L¹ is —C(O)—, L² is —S(O)₂—, and X is a substituted or unsubstituted thienyl.

In another embodiment of the compounds of formula (IC), L¹ is —C(O)—, L² is —S(O)—, and X is a substituted or unsubstituted heteroaryl.

In another embodiment of the compounds of formula (IC), L¹ is —C(O)—, L² is —S(O)—, and X is a substituted or unsubstituted pyridyl.

In another embodiment of the compounds of formula (IC), L¹ is —C(O)—, L² is —S(O)—, and X is a substituted or unsubstituted pyrimidyl.

In another embodiment of the compounds of formula (IC), L¹ is —C(O)—, L² is —S(O)—, and X is a substituted or unsubstituted benzimidazolyl.

In another embodiment of the compounds of formula (IC), L¹ is —C(O)—, L² is —S(O)—, and X is a substituted or unsubstituted quinolyl.

In another embodiment of the compounds of formula (IC), L¹ is —C(O)—, L² is —S(O)—, and X is a substituted or unsubstituted purinyl.

In another embodiment of the compounds of formula (IC), L¹ is —C(O)—, L² is —S(O)—, and X is a substituted or unsubstituted thienyl.

In another embodiment of the compounds of formula (IC), L¹ is —C(O)—, L² is —C(O)—, and X is a substituted or unsubstituted heteroaryl.

In another embodiment of the compounds of formula (IC), L¹ is —C(O)—, L² is —C(O)—, and X is a substituted or unsubstituted pyridyl.

In another embodiment of the compounds of formula (IC), L¹ is —C(O)—, L² is —C(O)—, and X is a substituted or unsubstituted pyrimidyl.

In another embodiment of the compounds of formula (IC), L¹ is —C(O)—, L² is —C(O)—, and X is a substituted or unsubstituted benzimidazolyl.

In another embodiment of the compounds of formula (IC), L¹ is —C(O)—, L² is —C(O)—, and X is a substituted or unsubstituted quinolyl.

In another embodiment of the compounds of formula (IC), L¹ is —C(O)—, L² is —C(O)—, and X is a substituted or unsubstituted purinyl.

In another embodiment of the compounds of formula (IC), L¹ is —C(O)—, L² is —C(O)—, and X is a substituted or unsubstituted thienyl.

In another embodiment of the compounds of formula (IC), L¹ is —S(O)₂—, L² is a covalent bond, and X is a substituted or unsubstituted heteroaryl.

In another embodiment of the compounds of formula (IC), L¹ is —S(O)₂—, L² is a covalent bond, and X is a substituted or unsubstituted pyridyl.

In another embodiment of the compounds of formula (IC), L¹ is —S(O)₂—, L² is a covalent bond, and X is a substituted or unsubstituted pyrimidyl.

In another embodiment of the compounds of formula (IC), L¹ is —S(O)₂—, L² is a covalent bond, and X is a substituted or unsubstituted benzimidazolyl.

In another embodiment of the compounds of formula (IC), L¹ is —S(O)₂—, L² is a covalent bond, and X is a substituted or unsubstituted quinolyl.

In another embodiment of the compounds of formula (IC), L¹ is —S(O)₂—, L² is a covalent bond, and X is a substituted or unsubstituted purinyl.

In another embodiment of the compounds of formula (IC), L¹ is —S(O)₂—, L² is a covalent bond, and X is a substituted or unsubstituted thienyl.

In another embodiment of the compounds of formula (IC), L¹ is —S(O)₂—, L² is —C(R⁴)₂—, and X is a substituted or unsubstituted heteroaryl.

In another embodiment of the compounds of formula (IC), L¹ is —S(O)₂—, L² is —C(R⁴)₂—, and X is a substituted or unsubstituted pyridyl.

In another embodiment of the compounds of formula (IC), L¹ is —S(O)₂—, L² is —C(R⁴)₂—, and X is a substituted or unsubstituted pyrimidyl.

In another embodiment of the compounds of formula (IC), L¹ is —S(O)₂—, L² is —C(R⁴)₂—, and X is a substituted or unsubstituted benzimidazolyl.

In another embodiment of the compounds of formula (IC), L¹ is —S(O)₂—, L² is —C(R⁴)₂—, and X is a substituted or unsubstituted quinolyl.

In another embodiment of the compounds of formula (IC), L¹ is —S(O)₂—, L² is —C(R⁴)₂—, and X is a substituted or unsubstituted purinyl.

In another embodiment of the compounds of formula (IC), L¹ is —S(O)₂—, L² is —C(R⁴)₂—, and X is a substituted or unsubstituted thienyl.

In another embodiment of the compounds of formula (IC), L¹ is —S(O)₂—, L² is —S(O)₂—, and X is a substituted or unsubstituted heteroaryl.

In another embodiment of the compounds of formula (IC), L¹ is —S(O)₂—, L² is —S(O)₂—, and X is a substituted or unsubstituted pyridyl.

In another embodiment of the compounds of formula (IC), L¹ is —S(O)₂—, L² is —S(O)₂—, and X is a substituted or unsubstituted pyrimidyl.

In another embodiment of the compounds of formula (IC), L¹ is —S(O)₂—, L² is —S(O)₂—, and X is a substituted or unsubstituted benzimidazolyl.

In another embodiment of the compounds of formula (IC), L¹ is —S(O)₂—, L² is —S(O)₂—, and X is a substituted or unsubstituted quinolyl.

In another embodiment of the compounds of formula (IC), L¹ is —S(O)₂—, L² is —S(O)₂—, and X is a substituted or unsubstituted purinyl.

In another embodiment of the compounds of formula (IC), L¹ is —S(O)₂—, L² is —S(O)₂—, and X is a substituted or unsubstituted thienyl.

In another embodiment of the compounds of formula (IC), L¹ is —S(O)₂—, L² is —S(O)—, and X is a substituted or unsubstituted heteroaryl.

In another embodiment of the compounds of formula (IC), L¹ is —S(O)₂—, L² is —S(O)—, and X is a substituted or unsubstituted pyridyl.

In another embodiment of the compounds of formula (IC), L¹ is —S(O)₂—, L² is —S(O)—, and X is a substituted or unsubstituted pyrimidyl.

In another embodiment of the compounds of formula (IC), L¹ is —S(O)₂—, L² is —S(O)—, and X is a substituted or unsubstituted benzimidazolyl.

In another embodiment of the compounds of formula (IC), L¹ is —S(O)₂—, L² is —S(O)—, and X is a substituted or unsubstituted quinolyl.

In another embodiment of the compounds of formula (IC), L¹ is —S(O)₂—, L² is —S(O)—, and X is a substituted or unsubstituted purinyl.

In another embodiment of the compounds of formula (IC), L¹ is —S(O)₂—, L² is —S(O)—, and X is a substituted or unsubstituted thienyl.

In another embodiment of the compounds of formula (IC), L¹ is —S(O)₂—, L² is —C(O)—, and X is a substituted or unsubstituted heteroaryl.

In another embodiment of the compounds of formula (IC), L¹ is —S(O)₂—, L² is —C(O)—, and X is a substituted or unsubstituted pyridyl.

In another embodiment of the compounds of formula (IC), L¹ is —S(O)₂—, L² is —C(O)—, and X is a substituted or unsubstituted pyrimidyl.

In another embodiment of the compounds of formula (IC), L¹ is —S(O)₂—, L² is —C(O)—, and X is a substituted or unsubstituted benzimidazolyl.

In another embodiment of the compounds of formula (IC), L¹ is —S(O)₂—, L² is —C(O)—, and X is a substituted or unsubstituted quinolyl.

In another embodiment of the compounds of formula (IC), L¹ is —S(O)₂—, L² is —C(O)—, and X is a substituted or unsubstituted purinyl.

In another embodiment of the compounds of formula (IC), L¹ is —S(O)₂—, L² is —C(O)—, and X is a substituted or unsubstituted thienyl.

In another embodiment of the compounds of formula (IC), L¹ is —S(O)—, L² is a covalent bond, and X is a substituted or unsubstituted heteroaryl.

In another embodiment of the compounds of formula (IC), L¹ is —S(O)—, L² is a covalent bond, and X is a substituted or unsubstituted pyridyl.

In another embodiment of the compounds of formula (IC), L¹ is —S(O)—, L² is a covalent bond, and X is a substituted or unsubstituted pyrimidyl.

In another embodiment of the compounds of formula (IC), L¹ is —S(O)—, L² is a covalent bond, and X is a substituted or unsubstituted benzimidazolyl.

In another embodiment of the compounds of formula (IC), L¹ is —S(O)—, L² is a covalent bond, and X is a substituted or unsubstituted quinolyl.

In another embodiment of the compounds of formula (IC), L¹ is —S(O)—, L² is a covalent bond, and X is a substituted or unsubstituted purinyl.

In another embodiment of the compounds of formula (IC), L¹ is —S(O)—, L² is a covalent bond, and X is a substituted or unsubstituted thienyl.

In another embodiment of the compounds of formula (IC), L¹ is —S(O)—, L² is —C(R⁴)₂—, and X is a substituted or unsubstituted heteroaryl.

In another embodiment of the compounds of formula (IC), L¹ is —S(O)—, L² is —C(R⁴)₂—, and X is a substituted or unsubstituted pyridyl.

In another embodiment of the compounds of formula (IC), L¹ is —S(O)—, L² is —C(R⁴)₂—, and X is a substituted or unsubstituted pyrimidyl.

In another embodiment of the compounds of formula (IC), L¹ is —S(O)—, L² is —C(R⁴)₂—, and X is a substituted or unsubstituted benzimidazolyl.

In another embodiment of the compounds of formula (IC), L¹ is —S(O)—, L² is —C(R⁴)₂—, and X is a substituted or unsubstituted quinolyl.

In another embodiment of the compounds of formula (IC), L¹ is —S(O)—, L² is —C(R⁴)₂—, and X is a substituted or unsubstituted purinyl.

In another embodiment of the compounds of formula (IC), L¹ is —S(O)—, L² is —C(R⁴)₂—, and X is a substituted or unsubstituted thienyl.

In another embodiment of the compounds of formula (IC), L¹ is —S(O)—, L² is —S(O)₂—, and X is a substituted or unsubstituted heteroaryl.

In another embodiment of the compounds of formula (IC), L¹ is —S(O)—, L² is —S(O)₂—, and X is a substituted or unsubstituted pyridyl.

In another embodiment of the compounds of formula (IC), L¹ is —S(O)—, L² is —S(O)₂—, and X is a substituted or unsubstituted pyrimidyl.

In another embodiment of the compounds of formula (IC), L¹ is —S(O)—, L² is —S(O)₂—, and X is a substituted or unsubstituted benzimidazolyl.

In another embodiment of the compounds of formula (IC), L¹ is —S(O)—, L² is —S(O)₂—, and X is a substituted or unsubstituted quinolyl.

In another embodiment of the compounds of formula (IC), L¹ is —S(O)—, L² is —S(O)₂—, and X is a substituted or unsubstituted purinyl.

In another embodiment of the compounds of formula (IC), L¹ is —S(O)—, L² is —S(O)₂—, and X is a substituted or unsubstituted thienyl.

In another embodiment of the compounds of formula (IC), L¹ is —S(O)—, L² is —S(O)—, and X is a substituted or unsubstituted heteroaryl.

In another embodiment of the compounds of formula (IC), L¹ is —S(O)—, L² is —S(O)—, and X is a substituted or unsubstituted pyridyl.

In another embodiment of the compounds of formula (IC), L¹ is —S(O)—, L² is —S(O)—, and X is a substituted or unsubstituted pyrimidyl.

In another embodiment of the compounds of formula (IC), L¹ is —S(O)—, L² is —S(O)—, and X is a substituted or unsubstituted benzimidazolyl.

In another embodiment of the compounds of formula (IC), L¹ is —S(O)—, L² is —S(O)—, and X is a substituted or unsubstituted quinolyl.

In another embodiment of the compounds of formula (IC), L¹ is —S(O)—, L² is —S(O)—, and X is a substituted or unsubstituted purinyl.

In another embodiment of the compounds of formula (IC), L¹ is —S(O)—, L² is —S(O)—, and X is a substituted or unsubstituted thienyl.

In another embodiment of the compounds of formula (IC), L¹ is —S(O)—, L² is —C(O)—, and X is a substituted or unsubstituted heteroaryl.

In another embodiment of the compounds of formula (IC), L¹ is —S(O)—, L² is —C(O)—, and X is a substituted or unsubstituted pyridyl.

In another embodiment of the compounds of formula (IC), L¹ is —S(O)—, L² is —C(O)—, and X is a substituted or unsubstituted pyrimidyl.

In another embodiment of the compounds of formula (IC), L¹ is —S(O)—, L² is —C(O)—, and X is a substituted or unsubstituted benzimidazolyl.

In another embodiment of the compounds of formula (IC), L¹ is —S(O)—, L² is —C(O)—, and X is a substituted or unsubstituted quinolyl.

In another embodiment of the compounds of formula (IC), L¹ is —S(O)—, L² is —C(O)—, and X is a substituted or unsubstituted purinyl.

In another embodiment of the compounds of formula (IC), L¹ is —S(O)—, L² is —C(O)—, and X is a substituted or unsubstituted thienyl.

In another embodiment of the compounds of formula (IC), L¹ is —S(O)₂—, L² is a covalent bond, and X is a substituted or unsubstituted heteroaryl.

In another embodiment of the compounds of formula (IC), L¹ is —S(O)₂—, L² is a covalent bond, and X is a substituted or unsubstituted pyridyl.

In another embodiment of the compounds of formula (IC), L¹ is —S(O)₂—, L² is a covalent bond, and X is a substituted or unsubstituted pyrimidyl.

In another embodiment of the compounds of formula (IC), L¹ is —S(O)₂—, L² is a covalent bond, and X is a substituted or unsubstituted benzimidazolyl.

In another embodiment of the compounds of formula (IC), L¹ is —S(O)₂—, L² is a covalent bond, and X is a substituted or unsubstituted quinolyl.

In another embodiment of the compounds of formula (IC), L¹ is —S(O)₂—, L² is a covalent bond, and X is a substituted or unsubstituted purinyl.

In another embodiment of the compounds of formula (IC), L¹ is —S(O)₂—, L² is a covalent bond, and X is a substituted or unsubstituted thienyl.

In another embodiment of the compounds of formula (IC), L¹ is —S(O)₂—, L² is —C(R⁴)₂—, and X is a substituted or unsubstituted heteroaryl.

In another embodiment of the compounds of formula (IC), L¹ is —S(O)₂—, L² is —C(R⁴)₂—, and X is a substituted or unsubstituted pyridyl.

In another embodiment of the compounds of formula (IC), L¹ is —S(O)₂—, L² is —C(R⁴)₂—, and X is a substituted or unsubstituted pyrimidyl.

In another embodiment of the compounds of formula (IC), L¹ is —S(O)₂—, L² is —C(R⁴)₂—, and X is a substituted or unsubstituted benzimidazolyl.

In another embodiment of the compounds of formula (IC), L¹ is —S(O)₂—, L² is —C(R⁴)₂—, and X is a substituted or unsubstituted quinolyl.

In another embodiment of the compounds of formula (IC), L¹ is —S(O)₂—, L² is —C(R⁴)₂—, and X is a substituted or unsubstituted purinyl.

In another embodiment of the compounds of formula (IC), L¹ is —S(O)₂—, L² is —C(R⁴)₂—, and X is a substituted or unsubstituted thienyl.

In another embodiment of the compounds of formula (IC), L¹ is —S(O)₂—, L² is —S(O)₂—, and X is a substituted or unsubstituted heteroaryl.

In another embodiment of the compounds of formula (IC), L¹ is —S(O)₂—, L² is —S(O)₂—, and X is a substituted or unsubstituted pyridyl.

In another embodiment of the compounds of formula (IC), L¹ is —S(O)₂—, L² is —S(O)₂—, and X is a substituted or unsubstituted pyrimidyl.

In another embodiment of the compounds of formula (IC), L¹ is —S(O)₂—, L² is —S(O)₂—, and X is a substituted or unsubstituted benzimidazolyl.

In another embodiment of the compounds of formula (IC), L¹ is —S(O)₂—, L² is —S(O)₂—, and X is a substituted or unsubstituted quinolyl.

In another embodiment of the compounds of formula (IC), L¹ is —S(O)₂—, L² is —S(O)₂—, and X is a substituted or unsubstituted purinyl.

In another embodiment of the compounds of formula (IC), L¹ is —S(O)₂—, L² is —S(O)₂—, and X is a substituted or unsubstituted thienyl.

In another embodiment of the compounds of formula (IC), L¹ is —S(O)₂—, L² is —S(O)—, and X is a substituted or unsubstituted heteroaryl.

In another embodiment of the compounds of formula (IC), L¹ is —S(O)₂—, L² is —S(O)—, and X is a substituted or unsubstituted pyridyl.

In another embodiment of the compounds of formula (IC), L¹ is —S(O)₂—, L² is —S(O)—, and X is a substituted or unsubstituted pyrimidyl.

In another embodiment of the compounds of formula (IC), L¹ is —S(O)₂—, L² is —S(O)—, and X is a substituted or unsubstituted benzimidazolyl.

In another embodiment of the compounds of formula (IC), L¹ is —S(O)₂—, L² is —S(O)—, and X is a substituted or unsubstituted quinolyl.

In another embodiment of the compounds of formula (IC), L¹ is —S(O)₂—, L² is —S(O)—, and X is a substituted or unsubstituted purinyl.

In another embodiment of the compounds of formula (IC), L¹ is —S(O)₂—, L² is —S(O)—, and X is a substituted or unsubstituted thienyl.

In another embodiment of the compounds of formula (IC), L¹ is —S(O)₂—, L² is —C(O)—, and X is a substituted or unsubstituted heteroaryl.

In another embodiment of the compounds of formula (IC), L¹ is —S(O)₂—, L² is —C(O)—, and X is a substituted or unsubstituted pyridyl.

In another embodiment of the compounds of formula (IC), L¹ is —S(O)₂—, L² is —C(O)—, and X is a substituted or unsubstituted pyrimidyl.

In another embodiment of the compounds of formula (IC), L¹ is —S(O)₂—, L² is —C(O)—, and X is a substituted or unsubstituted benzimidazolyl.

In another embodiment of the compounds of formula (IC), L¹ is —S(O)₂—, L² is —C(O)—, and X is a substituted or unsubstituted quinolyl.

In another embodiment of the compounds of formula (IC), L¹ is —S(O)₂—, L² is —C(O)—, and X is a substituted or unsubstituted purinyl.

In another embodiment of the compounds of formula (IC), L¹ is —S(O)₂—, L² is —C(O)—, and X is a substituted or unsubstituted thienyl.

In another embodiment of the compounds of formula (IC), L¹ is —C(O)—, L² is —CH₂—, and X is a substituted or unsubstituted heteroaryl.

In another embodiment of the compounds of formula (IC), L¹ is —C(O)—, L² is —CH₂—, and X is a substituted or unsubstituted pyridyl.

In another embodiment of the compounds of formula (IC), L¹ is —C(O)—, L² is —CH₂—, and X is a substituted or unsubstituted pyrimidyl.

In another embodiment of the compounds of formula (IC), L¹ is —C(O)—, L² is —CH₂—, and X is a substituted or unsubstituted benzimidazolyl.

In another embodiment of the compounds of formula (IC), L¹ is —C(O)—, L² is —CH₂—, and X is a substituted or unsubstituted quinolyl.

In another embodiment of the compounds of formula (IC), L¹ is —C(O)—, L² is —CH₂—, and X is a substituted or unsubstituted purinyl.

In another embodiment of the compounds of formula (IC), L¹ is —C(O)—, L² is —CH₂—, and X is a substituted or unsubstituted thienyl.

In another embodiment of the compounds of formula (IC), L¹ is —S(O)₂—, L² is —CH₂—, and X is a substituted or unsubstituted heteroaryl.

In another embodiment of the compounds of formula (IC), L¹ is —S(O)₂—, L² is —CH₂—, and X is a substituted or unsubstituted pyridyl.

In another embodiment of the compounds of formula (IC), L¹ is —S(O)₂—, L² is —CH₂—, and X is a substituted or unsubstituted pyrimidyl.

In another embodiment of the compounds of formula (IC), L¹ is —S(O)₂—, L² is —CH₂—, and X is a substituted or unsubstituted benzimidazolyl.

In another embodiment of the compounds of formula (IC), L¹ is —S(O)₂—, L² is —CH₂—, and X is a substituted or unsubstituted quinolyl.

In another embodiment of the compounds of formula (IC), L¹ is —S(O)₂—, L² is —CH₂—, and X is a substituted or unsubstituted purinyl.

In another embodiment of the compounds of formula (IC), L¹ is —S(O)₂—, L² is —CH₂—, and X is a substituted or unsubstituted thienyl.

In another embodiment of the compounds of formula (IC), L¹ is —S(O)—, L² is —CH₂—, and X is a substituted or unsubstituted heteroaryl.

In another embodiment of the compounds of formula (IC), L¹ is —S(O)—, L² is —CH₂—, and X is a substituted or unsubstituted pyridyl.

In another embodiment of the compounds of formula (IC), L¹ is —S(O)—, L² is —CH₂—, and X is a substituted or unsubstituted pyrimidyl.

In another embodiment of the compounds of formula (IC), L¹ is —S(O)—, L² is —CH₂—, and X is a substituted or unsubstituted benzimidazolyl.

In another embodiment of the compounds of formula (IC), L¹ is —S(O)—, L² is —CH₂—, and X is a substituted or unsubstituted quinolyl.

In another embodiment of the compounds of formula (IC), L¹ is —S(O)—, L² is —CH₂—, and X is a substituted or unsubstituted purinyl.

In another embodiment of the compounds of formula (IC), L¹ is —S(O)—, L² is —CH₂—, and X is a substituted or unsubstituted thienyl.

In another embodiment of the compounds of formula (IC), L¹ is —S(O)₂—, L² is —CH₂—, and X is a substituted or unsubstituted heteroaryl.

In another embodiment of the compounds of formula (IC), L¹ is —S(O)₂—, L² is —CH₂—, and X is a substituted or unsubstituted pyridyl.

In another embodiment of the compounds of formula (IC), L¹ is —S(O)₂—, L² is —CH₂—, and X is a substituted or unsubstituted pyrimidyl.

In another embodiment of the compounds of formula (IC), L¹ is —S(O)₂—, L² is —CH₂—, and X is a substituted or unsubstituted benzimidazolyl.

In another embodiment of the compounds of formula (IC), L¹ is —S(O)₂—, L² is —CH₂—, and X is a substituted or unsubstituted quinolyl.

In another embodiment of the compounds of formula (IC), L¹ is —S(O)₂—, L² is —CH₂—, and X is a substituted or unsubstituted purinyl.

In another embodiment of the compounds of formula (IC), L¹ is —S(O)₂—, L² is —CH₂—, and X is a substituted or unsubstituted thienyl.

In another embodiment of the compounds of formula (IC), L¹ is —C(O)—, L² is —C(R⁴)₂—, and X is substituted or unsubstituted amino.

In another embodiment of the compounds of formula (IC), L¹ is —C(O)—, L² is —CH₂—, and X is substituted or unsubstituted amino.

In another embodiment of the compounds of formula (IC), L¹ is —C(O)—, L² is —S(O)₂—, and X is substituted or unsubstituted amino.

In another embodiment of the compounds of formula (IC), L¹ is —C(O)—, L² is —S(O)—, and X is substituted or unsubstituted amino.

In another embodiment of the compounds of formula (IC), L¹ is —C(O)—, L² is —C(O)—, and X is substituted or unsubstituted amino.

In another embodiment of the compounds of formula (IC), L¹ is —S(O)₂—, L² is —C(R⁴)₂—, and X is substituted or unsubstituted amino.

In another embodiment of the compounds of formula (IC), L¹ is —S(O)₂—, L² is —CH₂—, and X is substituted or unsubstituted amino.

In another embodiment of the compounds of formula (IC), L¹ is —S(O)₂—, L² is —S(O)₂—, and X is substituted or unsubstituted amino.

In another embodiment of the compounds of formula (IC), L¹ is —S(O)₂—, L² is —S(O)—, and X is substituted or unsubstituted amino.

In another embodiment of the compounds of formula (IC), L¹ is —S(O)₂—, L² is —C(O)—, and X is substituted or unsubstituted amino.

In another embodiment of the compounds of formula (IC), L¹ is —S(O)—, L² is —C(R⁴)₂—, and X is substituted or unsubstituted amino.

In another embodiment of the compounds of formula (IC), L¹ is —S(O)—, L² is —CH₂—, and X is substituted or unsubstituted amino.

In another embodiment of the compounds of formula (IC), L¹ is —S(O)—, L² is —S(O)₂—, and X is substituted or unsubstituted amino.

In another embodiment of the compounds of formula (IC), L¹ is —S(O)—, L² is —S(O)—, and X is substituted or unsubstituted amino.

In another embodiment of the compounds of formula (IC), L¹ is —S(O)—, L² is —C(O)—, and X is substituted or unsubstituted amino.

In another embodiment of the compounds of formula (IC), L¹ is —S(O)₂—, L² is —C(R⁴)₂—, and X is substituted or unsubstituted amino.

In another embodiment of the compounds of formula (IC), L¹ is —S(O)₂—, L² is —CH₂—, and X is substituted or unsubstituted amino.

In another embodiment of the compounds of formula (IC), L¹ is —S(O)₂—, L² is —S(O)₂—, and X is substituted or unsubstituted amino.

In another embodiment of the compounds of formula (IC), L¹ is —S(O)₂—, L² is —S(O)—, and X is substituted or unsubstituted amino.

In another embodiment of the compounds of formula (IC), L¹ is —S(O)₂—, L² is —C(O)—, and X is substituted or unsubstituted amino.

In another embodiment of the compounds of formula (IC), L¹ is —C(O)—, L² is —C(R⁴)₂—, and X is —NH₂.

In another embodiment of the compounds of formula (IC), L¹ is —C(O)—, L² is —CH₂—, and X is —NH₂.

In another embodiment of the compounds of formula (IC), L¹ is —C(O)—, L² is —S(O)₂—, and X is —NH₂.

In another embodiment of the compounds of formula (IC), L¹ is —C(O)—, L² is —S(O)—, and X is —NH₂.

In another embodiment of the compounds of formula (IC), L¹ is —C(O)—, L² is —C(O)—, and X is —NH₂.

In another embodiment of the compounds of formula (IC), L¹ is —S(O)₂—, L² is —C(R⁴)₂—, and X is —NH₂.

In another embodiment of the compounds of formula (IC), L¹ is —S(O)₂—, L² is —CH₂—, and X is —NH₂.

In another embodiment of the compounds of formula (IC), L¹ is —S(O)₂—, L² is —S(O)₂—, and X is —NH₂.

In another embodiment of the compounds of formula (IC), L¹ is —S(O)₂—, L² is —S(O)—, and X is —NH₂.

In another embodiment of the compounds of formula (IC), L¹ is —S(O)₂—, L² is —C(O)—, and X is —NH₂.

In another embodiment of the compounds of formula (IC), L¹ is —S(O)—, L² is —C(R⁴)₂—, and X is —NH₂.

In another embodiment of the compounds of formula (IC), L¹ is —S(O)—, L² is —CH₂—, and X is —NH₂.

In another embodiment of the compounds of formula (IC), L¹ is —S(O)—, L² is —S(O)₂—, and X is —NH₂.

In another embodiment of the compounds of formula (IC), L¹ is —S(O)—, L² is —S(O)—, and X is —NH₂.

In another embodiment of the compounds of formula (IC), L¹ is —S(O)—, L² is —C(O)—, and X is —NH₂.

In another embodiment of the compounds of formula (IC), L¹ is —S(O)₂—, L² is —C(R⁴)₂—, and X is —NH₂.

In another embodiment of the compounds of formula (IC), L¹ is —S(O)₂—, L² is —CH₂—, and X is —NH₂.

In another embodiment of the compounds of formula (IC), L¹ is —S(O)₂—, L² is —S(O)₂—, and X is —NH₂.

In another embodiment of the compounds of formula (IC), L¹ is —S(O)₂—, L² is —S(O)—, and X is —NH₂.

In another embodiment of the compounds of formula (IC), L¹ is —S(O)₂—, L² is —C(O)—, and X is —NH₂.

In another embodiment of the compounds of formula (IC), L¹ is —C(O)—, L² is —C(R⁴)₂—, and X is —NHalkyl.

In another embodiment of the compounds of formula (IC), L¹ is —C(O)—, L² is —CH₂—, and X is —NHalkyl.

In another embodiment of the compounds of formula (IC), L¹ is —C(O)—, L² is —S(O)₂—, and X is —NHalkyl.

In another embodiment of the compounds of formula (IC), L¹ is —C(O)—, L² is —S(O)—, and X is —NHalkyl.

In another embodiment of the compounds of formula (IC), L¹ is —C(O)—, L² is —C(O)—, and X is —NHalkyl.

In another embodiment of the compounds of formula (IC), L¹ is —S(O)₂—, L² is —C(R⁴)₂—, and X is —NHalkyl.

In another embodiment of the compounds of formula (IC), L¹ is —S(O)₂—, L² is —CH₂—, and X is —NHalkyl.

In another embodiment of the compounds of formula (IC), L¹ is —S(O)₂—, L² is —S(O)₂—, and X is —NHalkyl.

In another embodiment of the compounds of formula (IC), L¹ is —S(O)₂—, L² is —S(O)—, and X is —NHalkyl.

In another embodiment of the compounds of formula (IC), L¹ is —S(O)₂—, L² is —C(O)—, and X is —NHalkyl.

In another embodiment of the compounds of formula (IC), L¹ is —S(O)—, L² is —C(R⁴)₂—, and X is —NHalkyl.

In another embodiment of the compounds of formula (IC), L¹ is —S(O)—, L² is —CH₂—, and X is —NHalkyl.

In another embodiment of the compounds of formula (IC), L¹ is —S(O)—, L² is —S(O)₂—, and X is —NHalkyl.

In another embodiment of the compounds of formula (IC), L¹ is —S(O)—, L² is —S(O)—, and X is —NHalkyl.

In another embodiment of the compounds of formula (IC), L¹ is —S(O)—, L² is —C(O)—, and X is —NHalkyl.

In another embodiment of the compounds of formula (IC), L¹ is —S(O)₂—, L² is —C(R⁴)₂—, and X is —NHalkyl.

In another embodiment of the compounds of formula (IC), L¹ is —S(O)₂—, L² is —CH₂—, and X is —NHalkyl.

In another embodiment of the compounds of formula (IC), L¹ is —S(O)₂—, L² is —S(O)₂—, and X is —NHalkyl.

In another embodiment of the compounds of formula (IC), L¹ is —S(O)₂—, L² is —S(O)—, and X is —NHalkyl.

In another embodiment of the compounds of formula (IC), L¹ is —S(O)₂—, L² is —C(O)—, and X is —NHalkyl.

In another embodiment of the compounds of formula (IC), L¹ is —C(O)—, L² is —C(R⁴)₂—, and X is substituted or unsubstituted —NHarylalkyl.

In another embodiment of the compounds of formula (IC), L¹ is —C(O)—, L² is —CH₂—, and X is substituted or unsubstituted —NHarylalkyl.

In another embodiment of the compounds of formula (IC), L¹ is —C(O)—, L² is —CH(alkyl)-, and X is substituted or unsubstituted —NHarylalkyl.

In another embodiment of the compounds of formula (IC), L¹ is —C(O)—, L² is —CH(CH₃)—, and X is substituted or unsubstituted —NHarylalkyl.

In another embodiment of the compounds of formula (IC), L¹ is —C(O)—, L² is —S(O)₂—, and X is substituted or unsubstituted —NHarylalkyl.

In another embodiment of the compounds of formula (IC), L¹ is —C(O)—, L² is —S(O)—, and X is substituted or unsubstituted —NHarylalkyl.

In another embodiment of the compounds of formula (IC), L¹ is —C(O)—, L² is —C(O)—, and X is substituted or unsubstituted —NHarylalkyl.

In another embodiment of the compounds of formula (IC), L¹ is —S(O)₂—, L² is —C(R⁴)₂—, and X is substituted or unsubstituted —NHarylalkyl.

In another embodiment of the compounds of formula (IC), L¹ is —S(O)₂—, L² is —CH(alkyl)-, and X is substituted or unsubstituted —NHarylalkyl.

In another embodiment of the compounds of formula (IC), L¹ is —S(O)₂—, L² is —CH(CH₃)—, and X is substituted or unsubstituted —NHarylalkyl.

In another embodiment of the compounds of formula (IC), L¹ is —S(O)₂—, L² is —CH₂—, and X is substituted or unsubstituted —NHarylalkyl.

In another embodiment of the compounds of formula (IC), L¹ is —S(O)₂—, L² is —S(O)₂—, and X is substituted or unsubstituted —NHarylalkyl.

In another embodiment of the compounds of formula (IC), L¹ is —S(O)₂—, L² is —S(O)—, and X is substituted or unsubstituted —NHarylalkyl.

In another embodiment of the compounds of formula (IC), L¹ is —S(O)₂—, L² is —C(O)—, and X is substituted or unsubstituted —NHarylalkyl.

In another embodiment of the compounds of formula (IC), L¹ is —S(O)—, L² is —C(R⁴)₂—, and X is substituted or unsubstituted —NHarylalkyl.

In another embodiment of the compounds of formula (IC), L¹ is —S(O)—, L² is —CH₂—, and X is substituted or unsubstituted —NHarylalkyl.

In another embodiment of the compounds of formula (IC), L¹ is —S(O)—, L² is —CH(alkyl)-, and X is substituted or unsubstituted —NHarylalkyl.

In another embodiment of the compounds of formula (IC), L¹ is —S(O)—, L² is —CH(CH₃)—, and X is substituted or unsubstituted —NHarylalkyl.

In another embodiment of the compounds of formula (IC), L¹ is —S(O)—, L² is —S(O)₂—, and X is substituted or unsubstituted —NHarylalkyl.

In another embodiment of the compounds of formula (IC), L¹ is —S(O)—, L² is —S(O)—, and X is substituted or unsubstituted —NHarylalkyl.

In another embodiment of the compounds of formula (IC), L¹ is —S(O)—, L² is —C(O)—, and X is substituted or unsubstituted —NHarylalkyl.

In another embodiment of the compounds of formula (IC), L¹ is —S(O)₂—, L² is —C(R⁴)₂—, and X is substituted or unsubstituted —NHarylalkyl.

In another embodiment of the compounds of formula (IC), L¹ is —S(O)₂—, L² is —CH₂—, and X is substituted or unsubstituted —NHarylalkyl.

In another embodiment of the compounds of formula (IC), L¹ is —S(O)₂—, L² is —CH(alkyl)-, and X is substituted or unsubstituted —NHarylalkyl.

In another embodiment of the compounds of formula (IC), L¹ is —S(O)₂—, L² is —CH(CH₃)—, and X is substituted or unsubstituted —NHarylalkyl.

In another embodiment of the compounds of formula (IC), L¹ is —S(O)₂—, L² is —S(O)₂—, and X is substituted or unsubstituted —NHarylalkyl.

In another embodiment of the compounds of formula (IC), L¹ is —S(O)₂—, L² is —S(O)—, and X is substituted or unsubstituted —NHarylalkyl.

In another embodiment of the compounds of formula (IC), L¹ is —S(O)₂—, L² is —C(O)—, and X is substituted or unsubstituted —NHarylalkyl.

In another embodiment of the compounds of formula (IC), L¹ is —C(O)—, L² is —C(R⁴)₂—, and X is substituted or unsubstituted —NHbenzyl.

In another embodiment of the compounds of formula (IC), L¹ is —C(O)—, L² is —CH₂—, and X is substituted or unsubstituted —NHbenzyl.

In another embodiment of the compounds of formula (IC), L¹ is —C(O)—, L² is —CH(alkyl)-, and X is substituted or unsubstituted —NHbenzyl.

In another embodiment of the compounds of formula (IC), L¹ is —C(O)—, L² is —CH(CH₃)—, and X is substituted or unsubstituted —NHbenzyl.

In another embodiment of the compounds of formula (IC), L¹ is —C(O)—, L² is —S(O)₂—, and X is substituted or unsubstituted —NHbenzyl.

In another embodiment of the compounds of formula (IC), L¹ is —C(O)—, L² is —S(O)—, and X is substituted or unsubstituted —NHbenzyl.

In another embodiment of the compounds of formula (IC), L¹ is —C(O)—, L² is —C(O)—, and X is substituted or unsubstituted —NHbenzyl.

In another embodiment of the compounds of formula (IC), L¹ is —S(O)₂—, L² is —C(R⁴)₂—, and X is substituted or unsubstituted —NHbenzyl.

In another embodiment of the compounds of formula (IC), L¹ is —S(O)₂—, L² is —CH(alkyl)-, and X is substituted or unsubstituted —NHbenzyl.

In another embodiment of the compounds of formula (IC), L¹ is —S(O)₂—, L² is —CH(CH₃)—, and X is substituted or unsubstituted —NHbenzyl.

In another embodiment of the compounds of formula (IC), L¹ is —S(O)₂—, L² is —CH₂—, and X is substituted or unsubstituted —NHbenzyl.

In another embodiment of the compounds of formula (IC), L¹ is —S(O)₂—, L² is —S(O)₂—, and X is substituted or unsubstituted —NHbenzyl.

In another embodiment of the compounds of formula (IC), L¹ is —S(O)₂—, L² is —S(O)—, and X is substituted or unsubstituted —NHbenzyl.

In another embodiment of the compounds of formula (IC), L¹ is —S(O)₂—, L² is —C(O)—, and X is substituted or unsubstituted —NHbenzyl.

In another embodiment of the compounds of formula (IC), L¹ is —S(O)—, L² is —C(R⁴)₂—, and X is substituted or unsubstituted —NHbenzyl.

In another embodiment of the compounds of formula (IC), L¹ is —S(O)—, L² is —CH₂—, and X is substituted or unsubstituted —NHbenzyl.

In another embodiment of the compounds of formula (IC), L¹ is —S(O)—, L² is —CH(alkyl)-, and X is substituted or unsubstituted —NHbenzyl.

In another embodiment of the compounds of formula (IC), L¹ is —S(O)—, L² is —CH(CH₃)—, and X is substituted or unsubstituted —NHbenzyl.

In another embodiment of the compounds of formula (IC), L¹ is —S(O)—, L² is —S(O)₂—, and X is substituted or unsubstituted —NHbenzyl.

In another embodiment of the compounds of formula (IC), L¹ is —S(O)—, L² is —S(O)—, and X is substituted or unsubstituted —NHbenzyl.

In another embodiment of the compounds of formula (IC), L¹ is —S(O)—, L² is —C(O)—, and X is substituted or unsubstituted —NHbenzyl.

In another embodiment of the compounds of formula (IC), L¹ is —S(O)₂—, L² is —C(R⁴)₂—, and X is substituted or unsubstituted —NHbenzyl.

In another embodiment of the compounds of formula (IC), L¹ is —S(O)₂—, L² is —CH₂—, and X is substituted or unsubstituted —NHbenzyl.

In another embodiment of the compounds of formula (IC), L¹ is —S(O)₂—, L² is —CH(alkyl)-, and X is substituted or unsubstituted —NHbenzyl.

In another embodiment of the compounds of formula (IC), L¹ is —S(O)₂—, L² is —CH(CH₃)—, and X is substituted or unsubstituted —NHbenzyl.

In another embodiment of the compounds of formula (IC), L¹ is —S(O)₂—, L² is —S(O)₂—, and X is substituted or unsubstituted —NHbenzyl.

In another embodiment of the compounds of formula (IC), L¹ is —S(O)₂—, L² is —S(O)—, and X is substituted or unsubstituted —NHbenzyl.

In another embodiment of the compounds of formula (IC), L¹ is —S(O)₂—, L² is —C(O)—, and X is substituted or unsubstituted —NHbenzyl.

In another embodiment of the compounds of formula (IC), L¹ is —C(O)—, L² is —C(R⁴)₂—, and X is substituted or unsubstituted —NHnaphthyl.

In another embodiment of the compounds of formula (IC), L¹ is —C(O)—, L² is —CH₂—, and X is substituted or unsubstituted —NHnaphthyl.

In another embodiment of the compounds of formula (IC), L¹ is —C(O)—, L² is —CH(alkyl)-, and X is substituted or unsubstituted —NHnaphthyl.

In another embodiment of the compounds of formula (IC), L¹ is —C(O)—, L² is —CH(CH₃)—, and X is substituted or unsubstituted —NHnaphthyl.

In another embodiment of the compounds of formula (IC), L¹ is —C(O)—, L² is —S(O)₂—, and X is substituted or unsubstituted —NHnaphthyl.

In another embodiment of the compounds of formula (IC), L¹ is —C(O)—, L² is —S(O)—, and X is substituted or unsubstituted —NHnaphthyl.

In another embodiment of the compounds of formula (IC), L¹ is —C(O)—, L² is —C(O)—, and X is substituted or unsubstituted —NHnaphthyl.

In another embodiment of the compounds of formula (IC), L¹ is —S(O)₂—, L² is —C(R⁴)₂—, and X is substituted or unsubstituted —NHnaphthyl.

In another embodiment of the compounds of formula (IC), L¹ is —S(O)₂—, L² is —CH(alkyl)-, and X is substituted or unsubstituted —NHnaphthyl.

In another embodiment of the compounds of formula (IC), L¹ is —S(O)₂—, L² is —CH(CH₃)—, and X is substituted or unsubstituted —NHnaphthyl.

In another embodiment of the compounds of formula (IC), L¹ is —S(O)₂—, L² is —CH₂—, and X is substituted or unsubstituted —NHnaphthyl.

In another embodiment of the compounds of formula (IC), L¹ is —S(O)₂—, L² is —S(O)₂—, and X is substituted or unsubstituted —NHnaphthyl.

In another embodiment of the compounds of formula (IC), L¹ is —S(O)₂—, L² is —S(O)—, and X is substituted or unsubstituted —NHnaphthyl.

In another embodiment of the compounds of formula (IC), L¹ is —S(O)₂—, L² is —C(O)—, and X is substituted or unsubstituted —NHnaphthyl.

In another embodiment of the compounds of formula (IC), L¹ is —S(O)—, L² is —C(R⁴)₂—, and X is substituted or unsubstituted —NHnaphthyl.

In another embodiment of the compounds of formula (IC), L¹ is —S(O)—, L² is —CH₂—, and X is substituted or unsubstituted —NHnaphthyl.

In another embodiment of the compounds of formula (IC), L¹ is —S(O)—, L² is —CH(alkyl)-, and X is substituted or unsubstituted —NHnaphthyl.

In another embodiment of the compounds of formula (IC), L¹ is —S(O)—, L² is —CH(CH₃)—, and X is substituted or unsubstituted —NHnaphthyl.

In another embodiment of the compounds of formula (IC), L¹ is —S(O)—, L² is —S(O)₂—, and X is substituted or unsubstituted —NHnaphthyl.

In another embodiment of the compounds of formula (IC), L¹ is —S(O)—, L² is —S(O)—, and X is substituted or unsubstituted —NHnaphthyl.

In another embodiment of the compounds of formula (IC), L¹ is —S(O)—, L² is —C(O)—, and X is substituted or unsubstituted —NHnaphthyl.

In another embodiment of the compounds of formula (IC), L¹ is —S(O)₂—, L² is —C(R⁴)₂—, and X is substituted or unsubstituted —NHnaphthyl.

In another embodiment of the compounds of formula (IC), L¹ is —S(O)₂—, L² is —CH₂—, and X is substituted or unsubstituted —NHnaphthyl.

In another embodiment of the compounds of formula (IC), L¹ is —S(O)₂—, L² is —CH(alkyl)-, and X is substituted or unsubstituted —NHnaphthyl.

In another embodiment of the compounds of formula (IC), L¹ is —S(O)₂—, L² is —CH(CH₃)—, and X is substituted or unsubstituted —NHnaphthyl.

In another embodiment of the compounds of formula (IC), L¹ is —S(O)₂—, L² is —S(O)₂—, and X is substituted or unsubstituted —NHnaphthyl.

In another embodiment of the compounds of formula (IC), L¹ is —S(O)₂—, L² is —S(O)—, and X is substituted or unsubstituted —NHnaphthyl.

In another embodiment of the compounds of formula (IC), L¹ is —S(O)₂—, L² is —C(O)—, and X is substituted or unsubstituted —NHnaphthyl.

In another embodiment of the compounds of formula (IC), L¹ is —C(O)— and R⁵ and -L²-X together with the nitrogen atom to which they are both attached form a substituted or unsubstituted heterocyclyl ring.

In another embodiment of the compounds of formula (IC), L¹ is —C(O)— and R⁵ and -L²-X together with the nitrogen atom to which they are both attached form a substituted or unsubstituted morpholinyl ring.

In another embodiment of the compounds of formula (IC), L¹ is —C(O)— and R⁵ and -L²-X together with the nitrogen atom to which they are both attached form a substituted or unsubstituted pyrrolidinyl ring.

In another embodiment of the compounds of formula (IC), L¹ is —C(O)— and R⁵ and -L²-X together with the nitrogen atom to which they are both attached form a substituted or unsubstituted piperidinyl ring.

In another embodiment of the compounds of formula (IC), L¹ is —C(O)— and R⁵ and -L²-X together with the nitrogen atom to which they are both attached form a substituted or unsubstituted piperazinyl ring.

In another embodiment of the compounds of formula (IC), L¹ is —S(O)₂— and R⁵ and -L²-X together with the nitrogen atom to which they are both attached form a substituted or unsubstituted heterocyclyl ring.

In another embodiment of the compounds of formula (IC), L¹ is —S(O)₂— and R⁵ and -L²-X together with the nitrogen atom to which they are both attached form a substituted or unsubstituted morpholinyl ring.

In another embodiment of the compounds of formula (IC), L¹ is —S(O)₂— and R⁵ and -L²-X together with the nitrogen atom to which they are both attached form a substituted or unsubstituted pyrrolidinyl ring.

In another embodiment of the compounds of formula (IC), L¹ is —S(O)₂— and R⁵ and -L²-X together with the nitrogen atom to which they are both attached form a substituted or unsubstituted piperidinyl ring.

In another embodiment of the compounds of formula (IC), L¹ is —S(O)₂— and R⁵ and -L²-X together with the nitrogen atom to which they are both attached form a substituted or unsubstituted piperazinyl ring.

In another embodiment of the compounds of formula (IC), L¹ is —S(O)— and R⁵ and -L²-X together with the nitrogen atom to which they are both attached form a substituted or unsubstituted heterocyclyl ring.

In another embodiment of the compounds of formula (IC), L¹ is —S(O)— and R⁵ and -L²-X together with the nitrogen atom to which they are both attached form a substituted or unsubstituted morpholinyl ring.

In another embodiment of the compounds of formula (IC), L¹ is —S(O)— and R⁵ and -L²-X together with the nitrogen atom to which they are both attached form a substituted or unsubstituted pyrrolidinyl ring.

In another embodiment of the compounds of formula (IC), L¹ is —S(O)— and R⁵ and -L²-X together with the nitrogen atom to which they are both attached form a substituted or unsubstituted piperidinyl ring.

In another embodiment of the compounds of formula (IC), L¹ is —S(O)— and R⁵ and -L²-X together with the nitrogen atom to which they are both attached form a substituted or unsubstituted piperazinyl ring.

In another embodiment of the compounds of formula (IC), L¹ is —C(R⁴)₂— and R⁵ and -L²-X together with the nitrogen atom to which they are both attached form a substituted or unsubstituted heterocyclyl ring.

In another embodiment of the compounds of formula (IC), L¹ is —C(R⁴)₂— and R⁵ and -L²-X together with the nitrogen atom to which they are both attached form a substituted or unsubstituted morpholinyl ring.

In another embodiment of the compounds of formula (IC), L¹ is —C(R⁴)₂— and R⁵ and -L²-X together with the nitrogen atom to which they are both attached form a substituted or unsubstituted pyrrolidinyl ring.

In another embodiment of the compounds of formula (IC), L¹ is —C(R⁴)₂— and R⁵ and -L²-X together with the nitrogen atom to which they are both attached form a substituted or unsubstituted piperidinyl ring.

In another embodiment of the compounds of formula (IC), L¹ is —C(R⁴)₂— and R⁵ and -L²-X together with the nitrogen atom to which they are both attached form a substituted or unsubstituted piperazinyl ring.

In another embodiment, the compounds of the present invention, or pharmaceutically acceptable salts, solvates, prodrugs, tautomers, or esters thereof, have the structure of formula (IC-1):

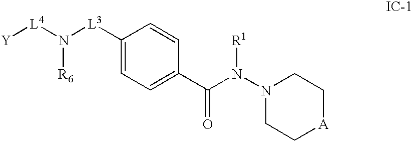

wherein R¹, R⁶, L³, L⁴ and Y are as defined herein, and A is —O—, —N(alkyl)-, —NH—, —N(arylalkyl)-, —N(heteroarylalkyl)-, or —N(acyl)-.

In another embodiment of the compounds of formula (IC-1), L⁴ is —C(R⁴)₂— or —C(O)—; R¹ is selected from the group consisting of H, alkyl, substituted or unsubstituted arylalkyl, aminoalkyl, and substituted or unsubstituted heteroarylalkyl; each R⁴ is independently selected from the group consisting of H and alkyl; and R⁶ is selected from the group consisting of H, alkyl, alkoxyalkyl, hydroxyalkyl, substituted or unsubstituted arylalkyl, and substituted or unsubstituted heteroarylalkyl; or R⁶ and -L⁴-Y, together with the nitrogen atom to which they are both attached, form a substituted or unsubstituted heterocyclyl ring.

In another embodiment of the compounds of formula (IC-1), L³ and L⁴ are each independently —CH₂— or —C(O)—; Y is selected from the group consisting of substituted or unsubstituted phenyl, substituted or unsubstituted thienyl, substituted or unsubstituted pyridyl, substituted or unsubstituted pyrimidyl, alkoxyalkyl; R¹ is selected from the group consisting of H, substituted or unsubstituted benzyl, aminopropyl, and substituted or unsubstituted benzimidazolyl; R⁶ is H or alkoxyalkyl; or R⁶ and -L⁴-Y, together with the nitrogen atom to which they are both attached, form a substituted or unsubstituted heterocyclyl ring.

In another embodiment of the compounds of formula (IC-1), L³ is —C(R⁴)₂—.

In another embodiment of the compounds of formula (IC-1), L³ is —C(O)—.

In another embodiment of the compounds of formula (IC-1), L³ is —S(O)—.

In another embodiment of the compounds of formula (IC-1), L³ is —S(O)₂—.

In another embodiment of the compounds of formula (IC-1), L³ is —C(R⁴)₂— and L⁴ is —C(O)—.

In another embodiment of the compounds of formula (IC-1), L³ is —C(R⁴)₂— and L⁴ is —C(R⁴)₂—.

In another embodiment of the compounds of formula (IC-1), L³ is —C(O)— and L⁴ is —C(O)—.

In another embodiment of the compounds of formula (IC-1), L³ is —C(O)— and L⁴ is —C(R⁴)₂—.

In another embodiment of the compounds of formula (IC-1), L³ is —CH₂— and L⁴ is —C(O)—.

In another embodiment of the compounds of formula (IC-1), L³ is —CH₂— and L⁴ is —CH₂—.

In another embodiment of the compounds of formula (IC-1), L³ is —C(O)— and L⁴ is —C(O)—.

In another embodiment of the compounds of formula (IC-1), L³ is —C(O)— and L⁴ is —CH₂—.

In another embodiment of the compounds of formula (IC-1), L³ is —CH(alkyl)- and L⁴ is —C(O)—.

In another embodiment of the compounds of formula (IC-1), L³ is —CH(alkyl)- and L⁴ is —CH₂—.

In another embodiment of the compounds of formula (IC-1), L³ is —CH₂— and L⁴ is —CH(alkyl)-.

In another embodiment of the compounds of formula (IC-1), L³ is —CH(alkyl)- and L⁴ is —CH(alkyl)-.

In another embodiment of the compounds of formula (IC-1), L³ is —C(O)— and L⁴ is —C(O)—.

In another embodiment of the compounds of formula (IC-1), L³ is —C(O)— and L⁴ is —CH(alkyl)-.

In another embodiment of the compounds of formula (IC-1), L³ is —C(R⁴)₂—, L⁴ is —C(O)—, and Y is substituted or unsubstituted aryl.

In another embodiment of the compounds of formula (IC-1), L³ is —C(R⁴)₂—, L⁴ is —C(R⁴)₂—, and Y is substituted or unsubstituted aryl.

In another embodiment of the compounds of formula (IC-1), L³ is —C(O)—, L⁴ is —C(O)—, and Y is substituted or unsubstituted aryl.

In another embodiment of the compounds of formula (IC-1), L³ is —C(O)—, L⁴ is —C(R⁴)₂—, and Y is substituted or unsubstituted aryl.

In another embodiment of the compounds of formula (IC-1), L³ is —C(R⁴)₂—, L⁴ is —C(O)—, and Y is substituted or unsubstituted phenyl.

In another embodiment of the compounds of formula (IC-1), L³ is —C(R⁴)₂—, L⁴ is —C(R⁴)₂—, and Y is substituted or unsubstituted phenyl.

In another embodiment of the compounds of formula (IC-1), L³ is —C(O)—, L⁴ is —C(O)—, and Y is substituted or unsubstituted phenyl.

In another embodiment of the compounds of formula (IC-1), L³ is —C(O)—, L⁴ is —C(R⁴)₂—, and Y is substituted or unsubstituted phenyl.

In another embodiment of the compounds of formula (IC-1), L³ is —C(R⁴)₂—, L⁴ is —C(O)—, and Y is substituted or unsubstituted heteroaryl.

In another embodiment of the compounds of formula (IC-1), L³ is —C(R⁴)₂—, L⁴ is —C(O)—, and Y is substituted or unsubstituted pyridyl.

In another embodiment of the compounds of formula (IC-1), L³ is —C(R⁴)₂—, L⁴ is —C(O)—, and Y is substituted or unsubstituted pyrimidyl.

In another embodiment of the compounds of formula (IC-1), L³ is —C(R⁴)₂—, L⁴ is —C(O)—, and Y is substituted or unsubstituted quinolyl.

In another embodiment of the compounds of formula (IC-1), L³ is —C(R⁴)₂—, L⁴ is —C(O)—, and Y is substituted or unsubstituted thienyl.

In another embodiment of the compounds of formula (IC-1), L³ is —C(R⁴)₂—, L⁴ is —C(O)—, and Y is substituted or unsubstituted benzimidazolyl.

In another embodiment of the compounds of formula (IC-1), L³ is —C(R⁴)₂—, L⁴ is —C(O)—, and Y is substituted or unsubstituted purinyl.

In another embodiment of the compounds of formula (IC-1), L³ is —C(R⁴)₂—, L⁴ is —C(R⁴)₂—, and Y is substituted or unsubstituted heteroaryl.

In another embodiment of the compounds of formula (IC-1), L³ is —C(R⁴)₂—, L⁴ is —C(R⁴)₂—, and Y is substituted or unsubstituted pyridyl.

In another embodiment of the compounds of formula (IC-1), L³ is —C(R⁴)₂—, L⁴ is —C(R⁴)₂—, and Y is substituted or unsubstituted pyrimidyl.

In another embodiment of the compounds of formula (IC-1), L³ is —C(R⁴)₂—, L⁴ is —C(R⁴)₂—, and Y is substituted or unsubstituted quinolyl.

In another embodiment of the compounds of formula (IC-1), L³ is —C(R⁴)₂—, L⁴ is —C(R⁴)₂—, and Y is substituted or unsubstituted thienyl.

In another embodiment of the compounds of formula (IC-1), L³ is —C(R⁴)₂—, L⁴ is —C(R⁴)₂—, and Y is substituted or unsubstituted benzimidazolyl.

In another embodiment of the compounds of formula (IC-1), L³ is —C(R⁴)₂—, L⁴ is —C(R⁴)₂—, and Y is substituted or unsubstituted purinyl.

In another embodiment of the compounds of formula (IC-1), L³ is —C(O)—, L⁴ is —C(O)—, and Y is substituted or unsubstituted heteroaryl.

In another embodiment of the compounds of formula (IC-1), L³ is —C(O)—, L⁴ is —C(O)—, and Y is substituted or unsubstituted pyridyl.

In another embodiment of the compounds of formula (IC-1), L³ is —C(O)—, L⁴ is —C(O)—, and Y is substituted or unsubstituted pyrimidyl.

In another embodiment of the compounds of formula (IC-1), L³ is —C(O)—, L⁴ is —C(O)—, and Y is substituted or unsubstituted quinolyl.

In another embodiment of the compounds of formula (IC-1), L³ is —C(O)—, L⁴ is —C(O)—, and Y is substituted or unsubstituted thienyl.

In another embodiment of the compounds of formula (IC-1), L³ is —C(O)—, L⁴ is —C(O)—, and Y is substituted or unsubstituted benzimidazolyl.

In another embodiment of the compounds of formula (IC-1), L³ is —C(O)—, L⁴ is —C(O)—, and Y is substituted or unsubstituted purinyl.

In another embodiment of the compounds of formula (IC-1), L³ is —C(O)—, L⁴ is —C(R⁴)₂—, and Y is substituted or unsubstituted heteroaryl.

In another embodiment of the compounds of formula (IC-1), L³ is —C(O)—, L⁴ is —C(R⁴)₂—, and Y is substituted or unsubstituted pyridyl.

In another embodiment of the compounds of formula (IC-1), L³ is —C(O)—, L⁴ is —C(R⁴)₂—, and Y is substituted or unsubstituted pyrimidyl.

In another embodiment of the compounds of formula (IC-1), L³ is —C(O)—, L⁴ is —C(R⁴)₂—, and Y is substituted or unsubstituted quinolyl.

In another embodiment of the compounds of formula (IC-1), L³ is —C(O)—, L⁴ is —C(R⁴)₂—, and Y is substituted or unsubstituted thienyl.

In another embodiment of the compounds of formula (IC-1), L³ is —C(O)—, L⁴ is —C(R⁴)₂—, and Y is substituted or unsubstituted benzimidazolyl.

In another embodiment of the compounds of formula (IC-1), L³ is —C(O)—, L⁴ is —C(R⁴)₂—, and Y is substituted or unsubstituted purinyl.

In another embodiment of the compounds of formula (IC-1), L³ is —CH₂—, L⁴ is —C(O)—, and Y is substituted or unsubstituted aryl.

In another embodiment of the compounds of formula (IC-1), L³ is —CH(alkyl)-, L⁴ is —C(O)—, and Y is substituted or unsubstituted aryl.

In another embodiment of the compounds of formula (IC-1), L³ is —CH₂—, L⁴ is —C(R⁴)₂—, and Y is substituted or unsubstituted aryl.

In another embodiment of the compounds of formula (IC-1), L³ is —C(R⁴)₂—, L⁴ is —CH₂—, and Y is substituted or unsubstituted aryl.

In another embodiment of the compounds of formula (IC-1), L³ is —CH₂—, L⁴ is —CH₂—, and Y is substituted or unsubstituted aryl.

In another embodiment of the compounds of formula (IC-1), L³ is —CH(alkyl)-, L⁴ is —C(R⁴)₂—, and Y is substituted or unsubstituted aryl.

In another embodiment of the compounds of formula (IC-1), L³ is —C(R⁴)₂—, L⁴ is —CH(alkyl)-, and Y is substituted or unsubstituted aryl.

In another embodiment of the compounds of formula (IC-1), L³ is —CH₂—, L⁴ is —CH(alkyl)-, and Y is substituted or unsubstituted aryl.

In another embodiment of the compounds of formula (IC-1), L³ is —CH(alkyl)-, L⁴ is —CH₂—, and Y is substituted or unsubstituted aryl.

In another embodiment of the compounds of formula (IC-1), L³ is —CH(alkyl)-, L⁴ is —CH(alkyl)-, and Y is substituted or unsubstituted aryl.

In another embodiment of the compounds of formula (IC-1), L³ is —C(O)—, L⁴ is —CH(alkyl)-, and Y is substituted or unsubstituted aryl.

In another embodiment of the compounds of formula (IC-1), L³ is —CH(alkyl)-, L⁴ is —C(O)—, and Y is substituted or unsubstituted phenyl.

In another embodiment of the compounds of formula (IC-1), L³ is —C(R⁴)₂—, L⁴ is —C(R⁴)₂—, and Y is substituted or unsubstituted phenyl.

In another embodiment of the compounds of formula (IC-1), L³ is —CH₂—, L⁴ is —C(R⁴)₂—, and Y is substituted or unsubstituted phenyl.

In another embodiment of the compounds of formula (IC-1), L³ is —C(R⁴)₂—, L⁴ is —CH₂—, and Y is substituted or unsubstituted phenyl.

In another embodiment of the compounds of formula (IC-1), L³ is —CH₂—, L⁴ is —CH₂—, and Y is substituted or unsubstituted phenyl.

In another embodiment of the compounds of formula (IC-1), L³ is —CH(alkyl)-, L⁴ is —C(R⁴)₂—, and Y is substituted or unsubstituted phenyl.

In another embodiment of the compounds of formula (IC-1), L³ is —C(R⁴)₂—, L⁴ is —CH(alkyl)-, and Y is substituted or unsubstituted phenyl.

In another embodiment of the compounds of formula (IC-1), L³ is —CH₂—, L⁴ is —CH(alkyl)-, and Y is substituted or unsubstituted phenyl.

In another embodiment of the compounds of formula (IC-1), L³ is —CH(alkyl)-, L⁴ is —CH₂—, and Y is substituted or unsubstituted phenyl.

In another embodiment of the compounds of formula (IC-1), L³ is —CH(alkyl)-, L⁴ is —CH(alkyl)-, and Y is substituted or unsubstituted phenyl.

In another embodiment of the compounds of formula (IC-1), L³ is —C(O)—, L⁴ is —CH₂—, and Y is substituted or unsubstituted phenyl.

In another embodiment of the compounds of formula (IC-1), L³ is —C(O)—, L⁴ is —CH(alkyl)-, and Y is substituted or unsubstituted phenyl.

In another embodiment of the compounds of formula (IC-1), L³ is —CH₂—, L⁴ is —C(O)—, and Y is substituted or unsubstituted heteroaryl.

In another embodiment of the compounds of formula (IC-1), L³ is —CH(alkyl)-, L⁴ is —C(O)—, and Y is substituted or unsubstituted heteroaryl.

In another embodiment of the compounds of formula (IC-1), L³ is —CH₂—, L⁴ is —C(O)—, and Y is substituted or unsubstituted pyridyl.

In another embodiment of the compounds of formula (IC-1), L³ is —CH(alkyl)-, L⁴ is —C(O)—, and Y is substituted or unsubstituted pyridyl.

In another embodiment of the compounds of formula (IC-1), L³ is —CH₂—, L⁴ is —C(O)—, and Y is substituted or unsubstituted pyrimidyl.

In another embodiment of the compounds of formula (IC-1), L³ is —CH(alkyl)-, L⁴ is —C(O)—, and Y is substituted or unsubstituted pyrimidyl.

In another embodiment of the compounds of formula (IC-1), L³ is —CH₂—, L⁴ is —C(O)—, and Y is substituted or unsubstituted quinolyl.

In another embodiment of the compounds of formula (IC-1), L³ is —CH(alkyl)-, L⁴ is —C(O)—, and Y is substituted or unsubstituted quinolyl.

In another embodiment of the compounds of formula (IC-1), L³ is —CH₂—, L⁴ is —C(O)—, and Y is substituted or unsubstituted thienyl.

In another embodiment of the compounds of formula (IC-1), L³ is —CH(alkyl)-, L⁴ is —C(O)—, and Y is substituted or unsubstituted thienyl.

In another embodiment of the compounds of formula (IC-1), L³ is —CH₂—, L⁴ is —C(O)—, and Y is substituted or unsubstituted benzimidazolyl.

In another embodiment of the compounds of formula (IC-1), L³ is —CH(alkyl)-, L⁴ is —C(O)—, and Y is substituted or unsubstituted benzimidazolyl.

In another embodiment of the compounds of formula (IC-1), L³ is —CH₂—, L⁴ is —C(O)—, and Y is substituted or unsubstituted purinyl.

In another embodiment of the compounds of formula (IC-1), L³ is —CH(alkyl)-, L⁴ is —C(O)—, and Y is substituted or unsubstituted purinyl.

In another embodiment of the compounds of formula (IC-1), L³ is —CH₂—, L⁴ is —C(R⁴)₂—, and Y is substituted or unsubstituted heteroaryl.

In another embodiment of the compounds of formula (IC-1), L³ is —C(R⁴)₂—, L⁴ is —CH₂—, and Y is substituted or unsubstituted heteroaryl.

In another embodiment of the compounds of formula (IC-1), L³ is —CH₂—, L⁴ is —CH₂—, and Y is substituted or unsubstituted heteroaryl.

In another embodiment of the compounds of formula (IC-1), L³ is —CH(alkyl)-, L⁴ is —CH₂—, and Y is substituted or unsubstituted heteroaryl.

In another embodiment of the compounds of formula (IC-1), L³ is —CH₂—, L⁴ is —CH(alkyl)-, and Y is substituted or unsubstituted heteroaryl.

In another embodiment of the compounds of formula (IC-1), L³ is —CH(alkyl)-, L⁴ is —CH(alkyl)-, and Y is substituted or unsubstituted heteroaryl.

In another embodiment of the compounds of formula (IC-1), L³ is —C(R⁴)₂—, L⁴ is —C(R⁴)₂—, and Y is substituted or unsubstituted pyridyl.

In another embodiment of the compounds of formula (IC-1), L³ is —CH₂—, L⁴ is —C(R⁴)₂—, and Y is substituted or unsubstituted pyridyl.

In another embodiment of the compounds of formula (IC-1), L³ is —C(R⁴)₂—, L⁴ is —CH₂—, and Y is substituted or unsubstituted pyridyl.

In another embodiment of the compounds of formula (IC-1), L³ is —CH₂—, L⁴ is —CH₂—, and Y is substituted or unsubstituted pyridyl.

In another embodiment of the compounds of formula (IC-1), L³ is —CH(alkyl)-, L⁴ is —CH₂—, and Y is substituted or unsubstituted pyridyl.

In another embodiment of the compounds of formula (IC-1), L³ is —CH₂—, L⁴ is —CH(alkyl)-, and Y is substituted or unsubstituted pyridyl.

In another embodiment of the compounds of formula (IC-1), L³ is —CH(alkyl)-, L⁴ is —CH(alkyl)-, and Y is substituted or unsubstituted pyridyl.

In another embodiment of the compounds of formula (IC-1), L³ is —CH₂—, L⁴ is —C(R⁴)₂—, and Y is substituted or unsubstituted pyrimidyl.

In another embodiment of the compounds of formula (IC-1), L³ is —C(R⁴)₂—, L⁴ is —CH₂—, and Y is substituted or unsubstituted pyrimidyl.

In another embodiment of the compounds of formula (IC-1), L³ is —CH₂—, L⁴ is —CH₂—, and Y is substituted or unsubstituted pyrimidyl.

In another embodiment of the compounds of formula (IC-1), L³ is —CH(alkyl)-, L⁴ is —CH₂—, and Y is substituted or unsubstituted pyrimidyl.

In another embodiment of the compounds of formula (IC-1), L³ is —CH₂—, L⁴ is —CH(alkyl)-, and Y is substituted or unsubstituted pyrimidyl.

In another embodiment of the compounds of formula (IC-1), L³ is —CH(alkyl)-, L⁴ is —CH(alkyl)-, and Y is substituted or unsubstituted pyrimidyl.

In another embodiment of the compounds of formula (IC-1), L³ is —CH₂—, L⁴ is —C(R⁴)₂—, and Y is substituted or unsubstituted quinolyl.

In another embodiment of the compounds of formula (IC-1), L³ is —C(R⁴)₂—, L⁴ is —CH₂—, and Y is substituted or unsubstituted quinolyl.

In another embodiment of the compounds of formula (IC-1), L³ is —CH₂—, L⁴ is —CH₂—, and Y is substituted or unsubstituted quinolyl.

In another embodiment of the compounds of formula (IC-1), L³ is —CH(alkyl)-, L⁴ is —CH₂—, and Y is substituted or unsubstituted quinolyl.

In another embodiment of the compounds of formula (IC-1), L³ is —CH₂—, L⁴ is —CH(alkyl)-, and Y is substituted or unsubstituted quinolyl.

In another embodiment of the compounds of formula (IC-1), L³ is —CH(alkyl)-, L⁴ is —CH(alkyl)-, and Y is substituted or unsubstituted quinolyl.

In another embodiment of the compounds of formula (IC-1), L³ is —CH₂—, L⁴ is —C(R⁴)₂—, and Y is substituted or unsubstituted thienyl.

In another embodiment of the compounds of formula (IC-1), L³ is —C(R⁴)₂—, L⁴ is —CH₂—, and Y is substituted or unsubstituted thienyl.

In another embodiment of the compounds of formula (IC-1), L³ is —CH₂—, L⁴ is —CH₂—, and Y is substituted or unsubstituted thienyl.

In another embodiment of the compounds of formula (IC-1), L³ is —CH(alkyl)-, L⁴ is —CH₂—, and Y is substituted or unsubstituted thienyl.

In another embodiment of the compounds of formula (IC-1), L³ is —CH₂—, L⁴ is —CH(alkyl)-, and Y is substituted or unsubstituted thienyl.

In another embodiment of the compounds of formula (IC-1), L³ is —CH(alkyl)-, L⁴ is —CH(alkyl)-, and Y is substituted or unsubstituted thienyl.

In another embodiment of the compounds of formula (IC-1), L³ is —CH₂—, L⁴ is —C(R⁴)₂—, and Y is substituted or unsubstituted benzimidazolyl.

In another embodiment of the compounds of formula (IC-1), L³ is —C(R⁴)₂—, L⁴ is —CH₂—, and Y is substituted or unsubstituted benzimidazolyl.

In another embodiment of the compounds of formula (IC-1), L³ is —CH₂—, L⁴ is —CH₂—, and Y is substituted or unsubstituted benzimidazolyl.

In another embodiment of the compounds of formula (IC-1), L³ is —CH(alkyl)-, L⁴ is —CH₂—, and Y is substituted or unsubstituted benzimidazolyl.

In another embodiment of the compounds of formula (IC-1), L³ is —CH₂—, L⁴ is —CH(alkyl)-, and Y is substituted or unsubstituted benzimidazolyl.

In another embodiment of the compounds of formula (IC-1), L³ is —CH(alkyl)-, L⁴ is —CH(alkyl)-, and Y is substituted or unsubstituted benzimidazolyl.

In another embodiment of the compounds of formula (IC-1), L³ is —CH₂—, L⁴ is —C(R⁴)₂—, and Y is substituted or unsubstituted purinyl.

In another embodiment of the compounds of formula (IC-1), L³ is —C(R⁴)₂—, L⁴ is —CH₂—, and Y is substituted or unsubstituted purinyl.

In another embodiment of the compounds of formula (IC-1), L³ is —CH₂—, L⁴ is —CH₂—, and Y is substituted or unsubstituted purinyl.

In another embodiment of the compounds of formula (IC-1), L³ is —CH(alkyl)-, L⁴ is —CH₂—, and Y is substituted or unsubstituted purinyl.

In another embodiment of the compounds of formula (IC-1), L³ is —CH₂—, L⁴ is —CH(alkyl)-, and Y is substituted or unsubstituted purinyl.

In another embodiment of the compounds of formula (IC-1), L³ is —CH(alkyl)-, L⁴ is —CH(alkyl)-, and Y is substituted or unsubstituted purinyl.

In another embodiment of the compounds of formula (IC-1), L³ is —C(O)—, L⁴ is —CH₂—, and Y is substituted or unsubstituted heteroaryl.

In another embodiment of the compounds of formula (IC-1), L³ is —C(O)—, L⁴ is —CH(alkyl)-, and Y is substituted or unsubstituted heteroaryl.

In another embodiment of the compounds of formula (IC-1), L³ is —C(O)—, L⁴ is —CH₂—, and Y is substituted or unsubstituted pyridyl.

In another embodiment of the compounds of formula (IC-1), L³ is —C(O)—, L⁴ is —CH(alkyl)-, and Y is substituted or unsubstituted pyridyl.

In another embodiment of the compounds of formula (IC-1), L³ is —C(O)—, L⁴ is —CH₂—, and Y is substituted or unsubstituted pyrimidyl.

In another embodiment of the compounds of formula (IC-1), L³ is —C(O)—, L⁴ is —CH(alkyl)-, and Y is substituted or unsubstituted pyrimidyl.

In another embodiment of the compounds of formula (IC-1), L³ is —C(O)—, L⁴ is —CH₂—, and Y is substituted or unsubstituted quinolyl.

In another embodiment of the compounds of formula (IC-1), L³ is —C(O)—, L⁴ is —CH(alkyl)-, and Y is substituted or unsubstituted quinolyl.

In another embodiment of the compounds of formula (IC-1), L³ is —C(O)—, L⁴ is —CH₂—, and Y is substituted or unsubstituted thienyl.

In another embodiment of the compounds of formula (IC-1), L³ is —C(O)—, L⁴ is —CH(alkyl)-, and Y is substituted or unsubstituted thienyl.

In another embodiment of the compounds of formula (IC-1), L³ is —C(O)—, L⁴ is —CH₂—, and Y is substituted or unsubstituted benzimidazolyl.

In another embodiment of the compounds of formula (IC-1), L³ is —C(O)—, L⁴ is —CH(alkyl)-, and Y is substituted or unsubstituted benzimidazolyl.

In another embodiment of the compounds of formula (IC-1), L³ is —C(O)—, L⁴ is —CH₂—, and Y is substituted or unsubstituted purinyl.

In another embodiment of the compounds of formula (IC-1), L³ is —C(O)—, L⁴ is —CH(alkyl)-, and Y is substituted or unsubstituted purinyl.

In another embodiment of the compounds of formula (IC-1), R⁶ and -L⁴-Y, together with the nitrogen atom to which they are both attached, form a substituted or unsubstituted heterocyclyl ring.

In another embodiment of the compounds of formula (IC-1), R⁶ and -L⁴-Y, together with the nitrogen atom to which they are both attached, form a substituted or unsubstituted pyrrolidinyl ring.

In another embodiment of the compounds of formula (IC-1), R⁶ and -L⁴-Y, together with the nitrogen atom to which they are both attached, form a substituted or unsubstituted piperidinyl ring.

In another embodiment of the compounds of formula (IC-1), R⁶ and -L⁴-Y, together with the nitrogen atom to which they are both attached, form a substituted or unsubstituted piperazinyl ring.

In another embodiment of the compounds of formula (IC-1), R⁶ and -L⁴-Y, together with the nitrogen atom to which they are both attached, form a substituted or unsubstituted morpholinyl ring.

In another embodiment of the compounds of formula (IC-1), R⁶ and -L⁴-Y, together with the nitrogen atom to which they are both attached, form a substituted or unsubstituted tetraazacyclotetradecane ring.

In another embodiment of the compounds of formula (IC-1), R⁶ and -L⁴-Y, together with the nitrogen atom to which they are both attached, form a substituted or unsubstituted 4- to 18-membered ring containing at least one nitrogen atom in the ring.

In another embodiment of the compounds of formula (IC-1), R⁶ and -L⁴-Y, together with the nitrogen atom to which they are both attached, form a substituted or unsubstituted diazabicycloheptane.

In another embodiment of the compounds of formula (IC-1), R⁶ and -L⁴-Y, together with the nitrogen atom to which they are both attached, form a substituted or unsubstituted 2,5-diazabicyclo[2.2.1]heptane.

In another embodiment of the compounds of formula (IC-1), -L⁴-Y is an alkoxyalkyl.

In another embodiment of the compounds of formula (IC-1), -L⁴-Y is an alkoxyalkyl, wherein said alkoxyalkyl is any alkoxyalkyl described or defined herein.

In another embodiment of the compounds of formula (IC-1), -L⁴-Y is an alkoxyalkyl, and R⁶ is H.

In another embodiment of the compounds of formula (IC-1), -L⁴-Y and R⁶ are both alkoxyalkyl, wherein each alkoxyalkyl is independently any alkoxyalkyl described or defined herein.

In another embodiment of the compounds of formula (IC-1), -L⁴-Y is an alkoxyalkyl, and R⁶ is alkyl.

In another embodiment of the compounds of formula (IC-1), -L⁴-Y is an alkoxyalkyl, and R⁶ is substituted or unsubstituted cycloalkyl.

In another embodiment of the compounds of formula (IC-1), -L⁴-Y is an alkoxyalkyl, and R⁶ is substituted or unsubstituted heteroarylalkyl.

In another embodiment of the compounds of formula (IC-1), -L⁴-Y is an alkoxyalkyl, and R⁶ is substituted or unsubstituted arylalkyl.

In another embodiment of the compounds of formula (IC-1), R¹ is H and A is —O—.

In another embodiment of the compounds of formula (IC-1), R¹ is substituted or unsubstituted arylalkyl and A is —O—.

In another embodiment of the compounds of formula (IC-1), R¹ is substituted or unsubstituted benzyl and A is —O—.

In another embodiment of the compounds of formula (IC-1), R¹ is substituted or unsubstituted heteroarylalkyl and A is —O—.

In another embodiment of the compounds of formula (IC-1), R¹ is substituted or unsubstituted benzimidazolylmethyl and A is —O—.

In another embodiment of the compounds of formula (IC-1), R¹ is substituted or unsubstituted pyridylmethyl and A is —O—.

In another embodiment of the compounds of formula (IC-1), R¹ is substituted or unsubstituted pyrimidylmethyl and A is —O—.

In another embodiment of the compounds of formula (IC-1), R¹ is substituted or unsubstituted indolylmethyl and A is —O—.

In another embodiment of the compounds of formula (IC-1), R¹ is substituted or unsubstituted quinolylmethyl and A is —O—.

In another embodiment of the compounds of formula (IC-1), R¹ is aminoalkyl and A is —O—.

In another embodiment of the compounds of formula (IC-1), R¹ is aminomethyl and A is —O—.

In another embodiment of the compounds of formula (IC-1), R¹ is dialkylaminomethyl and A is —O—.

In another embodiment of the compounds of formula (IC-1), R¹ is substituted or unsubstituted diarylaminomethyl and A is —O—.

In another embodiment of the compounds of formula (IC-1), R¹ is H₂N-methyl and A is —O—.

In another embodiment of the compounds of formula (IC-1), R¹ is aminoethyl and A is —O—.

In another embodiment of the compounds of formula (IC-1), R¹ is dialkylaminoethyl and A is —O—.

In another embodiment of the compounds of formula (IC-1), R¹ is substituted or unsubstituted diarylaminoethyl and A is —O—.

In another embodiment of the compounds of formula (IC-1), R¹ is H₂N-ethyl and A is —O—.

In another embodiment of the compounds of formula (IC-1), R¹ is aminopropyl and A is —O—.

In another embodiment of the compounds of formula (IC-1), R¹ is dialkylaminopropyl and A is —O—.

In another embodiment of the compounds of formula (IC-1), R¹ is substituted or unsubstituted diarylaminopropyl and A is —O—.

In another embodiment of the compounds of formula (IC-1), R¹ is H₂N-propyl and A is —O—.

In another embodiment of the compounds of formula (IC-1), R¹ is H and A is —NH—.

In another embodiment of the compounds of formula (IC-1), R¹ is substituted or unsubstituted arylalkyl and A is —NH—.

In another embodiment of the compounds of formula (IC-1), R¹ is substituted or unsubstituted benzyl and A is —NH—.

In another embodiment of the compounds of formula (IC-1), R¹ is substituted or unsubstituted heteroarylalkyl and A is —NH—.

In another embodiment of the compounds of formula (IC-1), R¹ is substituted or unsubstituted benzimidazolylmethyl and A is —NH—.

In another embodiment of the compounds of formula (IC-1), R¹ is substituted or unsubstituted pyridylmethyl and A is —NH—.

In another embodiment of the compounds of formula (IC-1), R¹ is substituted or unsubstituted pyrimidylmethyl and A is —NH—.

In another embodiment of the compounds of formula (IC-1), R¹ is substituted or unsubstituted indolylmethyl and A is —NH—.

In another embodiment of the compounds of formula (IC-1), R¹ is substituted or unsubstituted quinolylmethyl and A is —NH—.

In another embodiment of the compounds of formula (IC-1), R¹ is aminoalkyl and A is —NH—.

In another embodiment of the compounds of formula (IC-1), R¹ is aminomethyl and A is —NH—.

In another embodiment of the compounds of formula (IC-1), R¹ is dialkylaminomethyl and A is —NH—.

In another embodiment of the compounds of formula (IC-1), R¹ is substituted or unsubstituted diarylaminomethyl and A is —NH—.

In another embodiment of the compounds of formula (IC-1), R¹ is H₂N-methyl and A is —NH—.

In another embodiment of the compounds of formula (IC-1), R¹ is aminoethyl and A is —NH—.

In another embodiment of the compounds of formula (IC-1), R¹ is dialkylaminoethyl and A is —NH—.

In another embodiment of the compounds of formula (IC-1), R¹ is substituted or unsubstituted diarylaminoethyl and A is —NH—.

In another embodiment of the compounds of formula (IC-1), R¹ is H₂N-ethyl and A is —NH—.

In another embodiment of the compounds of formula (IC-1), R¹ is aminopropyl and A is —NH—.

In another embodiment of the compounds of formula (IC-1), R¹ is dialkylaminopropyl and A is —NH—.

In another embodiment of the compounds of formula (IC-1), R¹ is substituted or unsubstituted diarylaminopropyl and A is —NH—.

In another embodiment of the compounds of formula (IC-1), R¹ is H₂N-propyl and A is —NH—.

In another embodiment of the compounds of formula (IC-1), R¹ is H and A is —N(alkyl)-.

In another embodiment of the compounds of formula (IC-1), R¹ is substituted or unsubstituted arylalkyl and A is —N(alkyl)-.

In another embodiment of the compounds of formula (IC-1), R¹ is substituted or unsubstituted benzyl and A is —N(alkyl)-.

In another embodiment of the compounds of formula (IC-1), R¹ is substituted or unsubstituted heteroarylalkyl and A is —N(alkyl)-.

In another embodiment of the compounds of formula (IC-1), R¹ is substituted or unsubstituted benzimidazolylmethyl and A is —N(alkyl)-.

In another embodiment of the compounds of formula (IC-1), R¹ is substituted or unsubstituted pyridylmethyl and A is —N(alkyl)-.

In another embodiment of the compounds of formula (IC-1), R¹ is substituted or unsubstituted pyrimidylmethyl and A is —N(alkyl)-.

In another embodiment of the compounds of formula (IC-1), R¹ is substituted or unsubstituted indolylmethyl and A is —N(alkyl)-.

In another embodiment of the compounds of formula (IC-1), R¹ is substituted or unsubstituted quinolylmethyl and A is —N(alkyl)-.

In another embodiment of the compounds of formula (IC-1), R¹ is aminoalkyl and A is —N(alkyl)-.

In another embodiment of the compounds of formula (IC-1), R¹ is aminomethyl and A is —N(alkyl)-.

In another embodiment of the compounds of formula (IC-1), R¹ is dialkylaminomethyl and A is —N(alkyl)-.

In another embodiment of the compounds of formula (IC-1), R¹ is substituted or unsubstituted diarylaminomethyl and A is —N(alkyl)-.

In another embodiment of the compounds of formula (IC-1), R¹ is H₂N-methyl and A is —N(alkyl)-.

In another embodiment of the compounds of formula (IC-1), R¹ is aminoethyl and A is —N(alkyl)-.

In another embodiment of the compounds of formula (IC-1), R¹ is dialkylaminoethyl and A is —N(alkyl)-.

In another embodiment of the compounds of formula (IC-1), R¹ is substituted or unsubstituted diarylaminoethyl and A is —N(alkyl)-.

In another embodiment of the compounds of formula (IC-1), R¹ is H₂N-ethyl and A is —N(alkyl)-.

In another embodiment of the compounds of formula (IC-1), R¹ is aminopropyl and A is —N(alkyl)-.

In another embodiment of the compounds of formula (IC-1), R¹ is dialkylaminopropyl and A is —N(alkyl)-.

In another embodiment of the compounds of formula (IC-1), R¹ is substituted or unsubstituted diarylaminopropyl and A is —N(alkyl)-.

In another embodiment of the compounds of formula (IC-1), R¹ is H₂N-propyl and A is —N(alkyl)-.

In another embodiment of the compounds of formula (IC-1), R¹ is H and A is —N(CH₃)—.

In another embodiment of the compounds of formula (IC-1), R¹ is substituted or unsubstituted arylalkyl and A is —N(CH₃)—.

In another embodiment of the compounds of formula (IC-1), R¹ is substituted or unsubstituted benzyl and A is —N(CH₃)—.

In another embodiment of the compounds of formula (IC-1), R¹ is substituted or unsubstituted heteroarylalkyl and A is —N(CH₃)—.

In another embodiment of the compounds of formula (IC-1), R¹ is substituted or unsubstituted benzimidazolylmethyl and A is —N(CH₃)—.

In another embodiment of the compounds of formula (IC-1), R¹ is substituted or unsubstituted pyridylmethyl and A is —N(CH₃)—.

In another embodiment of the compounds of formula (IC-1), R¹ is substituted or unsubstituted pyrimidylmethyl and A is —N(CH₃)—.

In another embodiment of the compounds of formula (IC-1), R¹ is substituted or unsubstituted indolylmethyl and A is —N(CH₃)—.

In another embodiment of the compounds of formula (IC-1), R¹ is substituted or unsubstituted quinolylmethyl and A is —N(CH₃)—.

In another embodiment of the compounds of formula (IC-1), R¹ is aminoalkyl and A is —N(CH₃)—.

In another embodiment of the compounds of formula (IC-1), R¹ is aminomethyl and A is —N(CH₃)—.

In another embodiment of the compounds of formula (IC-1), R¹ is dialkylaminomethyl and A is —N(CH₃)—.

In another embodiment of the compounds of formula (IC-1), R¹ is substituted or unsubstituted diarylaminomethyl and A is —N(CH₃)—.

In another embodiment of the compounds of formula (IC-1), R¹ is H₂N-methyl and A is —N(CH₃)—.

In another embodiment of the compounds of formula (IC-1), R¹ is aminoethyl and A is —N(CH₃)—.

In another embodiment of the compounds of formula (IC-1), R¹ is dialkylaminoethyl and A is —N(CH₃)—.

In another embodiment of the compounds of formula (IC-1), R¹ is substituted or unsubstituted diarylaminoethyl and A is —N(CH₃)—.

In another embodiment of the compounds of formula (IC-1), R¹ is H₂N-ethyl and A is —N(CH₃)—.

In another embodiment of the compounds of formula (IC-1), R¹ is aminopropyl and A is —N(CH₃)—.

In another embodiment of the compounds of formula (IC-1), R¹ is dialkylaminopropyl and A is —N(CH₃)—.

In another embodiment of the compounds of formula (IC-1), R¹ is substituted or unsubstituted diarylaminopropyl and A is —N(CH₃)—.

In another embodiment of the compounds of formula (IC-1), R¹ is H₂N-propyl and A is —N(CH₃)—.

In another embodiment of the compounds of formula (IC-1), R¹ is H and A is —N(CH₂CH₃)—.

In another embodiment of the compounds of formula (IC-1), R¹ is substituted or unsubstituted arylalkyl and A is —N(CH₂CH₃)—.

In another embodiment of the compounds of formula (IC-1), R¹ is substituted or unsubstituted benzyl and A is —N(CH₂CH₃)—.

In another embodiment of the compounds of formula (IC-1), R¹ is substituted or unsubstituted heteroarylalkyl and A is —N(CH₂CH₃)—.

In another embodiment of the compounds of formula (IC-1), R¹ is substituted or unsubstituted benzimidazolylmethyl and A is —N(CH₂CH₃)—.

In another embodiment of the compounds of formula (IC-1), R¹ is substituted or unsubstituted pyridylmethyl and A is —N(CH₂CH₃)—.

In another embodiment of the compounds of formula (IC-1), R¹ is substituted or unsubstituted pyrimidylmethyl and A is —N(CH₂CH₃)—.

In another embodiment of the compounds of formula (IC-1), R¹ is substituted or unsubstituted indolylmethyl and A is —N(CH₂CH₃)—.

In another embodiment of the compounds of formula (IC-1), R¹ is substituted or unsubstituted quinolylmethyl and A is —N(CH₂CH₃)—.

In another embodiment of the compounds of formula (IC-1), R¹ is aminoalkyl and A is —N(CH₂CH₃)—.

In another embodiment of the compounds of formula (IC-1), R¹ is aminomethyl and A is —N(CH₂CH₃)—.

In another embodiment of the compounds of formula (IC-1), R¹ is dialkylaminomethyl and A is —N(CH₂CH₃)—.

In another embodiment of the compounds of formula (IC-1), R¹ is substituted or unsubstituted diarylaminomethyl and A is —N(CH₂CH₃)—.

In another embodiment of the compounds of formula (IC-1), R¹ is H₂N-methyl and A is —N(CH₂CH₃)—.

In another embodiment of the compounds of formula (IC-1), R¹ is aminoethyl and A is —N(CH₂CH₃)—.

In another embodiment of the compounds of formula (IC-1), R¹ is dialkylaminoethyl and A is —N(CH₂CH₃)—.

In another embodiment of the compounds of formula (IC-1), R¹ is substituted or unsubstituted diarylaminoethyl and A is —N(CH₂CH₃)—.

In another embodiment of the compounds of formula (IC-1), R¹ is H₂N-ethyl and A is —N(CH₂CH₃)—.

In another embodiment of the compounds of formula (IC-1), R¹ is aminopropyl and A is —N(CH₂CH₃)—.

In another embodiment of the compounds of formula (IC-1), R¹ is dialkylaminopropyl and A is —N(CH₂CH₃)—.

In another embodiment of the compounds of formula (IC-1), R¹ is substituted or unsubstituted diarylaminopropyl and A is —N(CH₂CH₃)—.

In another embodiment of the compounds of formula (IC-1), R¹ is H₂N-propyl and A is —N(CH₂CH₃)—.

In another embodiment of the compounds of formula (IC-1), R¹ is H and A is —N(acyl)-.

In another embodiment of the compounds of formula (IC-1), R¹ is substituted or unsubstituted arylalkyl and A is —N(acyl)-.

In another embodiment of the compounds of formula (IC-1), R¹ is substituted or unsubstituted benzyl and A is —N(acyl)-.

In another embodiment of the compounds of formula (IC-1), R¹ is substituted or unsubstituted heteroarylalkyl and A is —N(acyl)-.

In another embodiment of the compounds of formula (IC-1), R¹ is substituted or unsubstituted benzimidazolylmethyl and A is —N(acyl)-.

In another embodiment of the compounds of formula (IC-1), R¹ is substituted or unsubstituted pyridylmethyl and A is —N(acyl)-.

In another embodiment of the compounds of formula (IC-1), R¹ is substituted or unsubstituted pyrimidylmethyl and A is —N(acyl)-.

In another embodiment of the compounds of formula (IC-1), R¹ is substituted or unsubstituted indolylmethyl and A is —N(acyl)-.

In another embodiment of the compounds of formula (IC-1), R¹ is substituted or unsubstituted quinolylmethyl and A is —N(acyl)-.

In another embodiment of the compounds of formula (IC-1), R¹ is aminoalkyl and A is —N(acyl)-.

In another embodiment of the compounds of formula (IC-1), R¹ is aminomethyl and A is —N(acyl)-.

In another embodiment of the compounds of formula (IC-1), R¹ is dialkylaminomethyl and A is —N(acyl)-.

In another embodiment of the compounds of formula (IC-1), R¹ is substituted or unsubstituted diarylaminomethyl and A is —N(acyl)-.

In another embodiment of the compounds of formula (IC-1), R¹ is H₂N-methyl and A is —N(acyl)-.

In another embodiment of the compounds of formula (IC-1), R¹ is aminoethyl and A is —N(acyl)-.

In another embodiment of the compounds of formula (IC-1), R¹ is dialkylaminoethyl and A is —N(acyl)-.

In another embodiment of the compounds of formula (IC-1), R¹ is substituted or unsubstituted diarylaminoethyl and A is —N(acyl)-.

In another embodiment of the compounds of formula (IC-1), R¹ is H₂N-ethyl and A is —N(acyl)-.

In another embodiment of the compounds of formula (IC-1), R¹ is aminopropyl and A is —N(acyl)-.

In another embodiment of the compounds of formula (IC-1), R¹ is dialkylaminopropyl and A is —N(acyl)-.

In another embodiment of the compounds of formula (IC-1), R¹ is substituted or unsubstituted diarylaminopropyl and A is —N(acyl)-.

In another embodiment of the compounds of formula (IC-1), R¹ is H₂N-propyl and A is —N(acyl)-.

In another embodiment of the compounds of formula (IC-1), R¹ is H and A is substituted or unsubstituted —N(arylalkyl)-.

In another embodiment of the compounds of formula (IC-1), R¹ is substituted or unsubstituted arylalkyl and A is substituted or unsubstituted —N(arylalkyl)-.

In another embodiment of the compounds of formula (IC-1), R¹ is substituted or unsubstituted benzyl and A is substituted or unsubstituted —N(arylalkyl)-.

In another embodiment of the compounds of formula (IC-1), R¹ is substituted or unsubstituted heteroarylalkyl and A is substituted or unsubstituted —N(arylalkyl)-.

In another embodiment of the compounds of formula (IC-1), R¹ is substituted or unsubstituted benzimidazolylmethyl and A is substituted or unsubstituted —N(arylalkyl)-.

In another embodiment of the compounds of formula (IC-1), R¹ is substituted or unsubstituted pyridylmethyl and A is substituted or unsubstituted —N(arylalkyl)-.

In another embodiment of the compounds of formula (IC-1), R¹ is substituted or unsubstituted pyrimidylmethyl and A is substituted or unsubstituted —N(arylalkyl)-.

In another embodiment of the compounds of formula (IC-1), R¹ is substituted or unsubstituted indolylmethyl and A is substituted or unsubstituted —N(arylalkyl)-.

In another embodiment of the compounds of formula (IC-1), R¹ is substituted or unsubstituted quinolylmethyl and A is substituted or unsubstituted —N(arylalkyl)-.

In another embodiment of the compounds of formula (IC-1), R¹ is aminoalkyl and A is substituted or unsubstituted —N(arylalkyl)-.

In another embodiment of the compounds of formula (IC-1), R¹ is aminomethyl and A is substituted or unsubstituted —N(arylalkyl)-.

In another embodiment of the compounds of formula (IC-1), R¹ is dialkylaminomethyl and A is substituted or unsubstituted —N(arylalkyl)-.

In another embodiment of the compounds of formula (IC-1), R¹ is substituted or unsubstituted diarylaminomethyl and A is substituted or unsubstituted —N(arylalkyl)-.

In another embodiment of the compounds of formula (IC-1), R¹ is H₂N-methyl and A is substituted or unsubstituted —N(arylalkyl)-.

In another embodiment of the compounds of formula (IC-1), R¹ is aminoethyl and A is substituted or unsubstituted —N(arylalkyl)-.

In another embodiment of the compounds of formula (IC-1), R¹ is dialkylaminoethyl and A is substituted or unsubstituted —N(arylalkyl)-.

In another embodiment of the compounds of formula (IC-1), R¹ is substituted or unsubstituted diarylaminoethyl and A is substituted or unsubstituted —N(arylalkyl)-.

In another embodiment of the compounds of formula (IC-1), R¹ is H₂N-ethyl and A is substituted or unsubstituted —N(arylalkyl)-.

In another embodiment of the compounds of formula (IC-1), R¹ is aminopropyl and A is substituted or unsubstituted —N(arylalkyl)-.

In another embodiment of the compounds of formula (IC-1), R¹ is dialkylaminopropyl and A is substituted or unsubstituted —N(arylalkyl)-.

In another embodiment of the compounds of formula (IC-1), R¹ is substituted or unsubstituted diarylaminopropyl and A is substituted or unsubstituted —N(arylalkyl)-.

In another embodiment of the compounds of formula (IC-1), R¹ is H₂N-propyl and A is substituted or unsubstituted —N(arylalkyl)-.

In another embodiment of the compounds of formula (IC-1), R¹ is H and A is substituted or unsubstituted —N(heteroarylalkyl)-.

In another embodiment of the compounds of formula (IC-1), R¹ is substituted or unsubstituted arylalkyl and A is substituted or unsubstituted —N(heteroarylalkyl)-.

In another embodiment of the compounds of formula (IC-1), R¹ is substituted or unsubstituted benzyl and A is substituted or unsubstituted —N(heteroarylalkyl)-.

In another embodiment of the compounds of formula (IC-1), R¹ is substituted or unsubstituted heteroarylalkyl and A is substituted or unsubstituted —N(heteroarylalkyl)-.

In another embodiment of the compounds of formula (IC-1), R¹ is substituted or unsubstituted benzimidazolylmethyl and A is substituted or unsubstituted —N(heteroarylalkyl)-.

In another embodiment of the compounds of formula (IC-1), R¹ is substituted or unsubstituted pyridylmethyl and A is substituted or unsubstituted —N(heteroarylalkyl)-.

In another embodiment of the compounds of formula (IC-1), R¹ is substituted or unsubstituted pyrimidylmethyl and A is substituted or unsubstituted —N(heteroarylalkyl)-.

In another embodiment of the compounds of formula (IC-1), R¹ is substituted or unsubstituted indolylmethyl and A is substituted or unsubstituted —N(heteroarylalkyl)-.

In another embodiment of the compounds of formula (IC-1), R¹ is substituted or unsubstituted quinolylmethyl and A is substituted or unsubstituted —N(heteroarylalkyl)-.

In another embodiment of the compounds of formula (IC-1), R¹ is aminoalkyl and A is substituted or unsubstituted —N(heteroarylalkyl)-.

In another embodiment of the compounds of formula (IC-1), R¹ is aminomethyl and A is substituted or unsubstituted —N(heteroarylalkyl)-.

In another embodiment of the compounds of formula (IC-1), R¹ is dialkylaminomethyl and A is substituted or unsubstituted —N(heteroarylalkyl)-.

In another embodiment of the compounds of formula (IC-1), R¹ is substituted or unsubstituted diarylaminomethyl and A is substituted or unsubstituted —N(heteroarylalkyl)-.

In another embodiment of the compounds of formula (IC-1), R¹ is H₂N-methyl and A is substituted or unsubstituted —N(heteroarylalkyl)-.

In another embodiment of the compounds of formula (IC-1), R¹ is aminoethyl and A is substituted or unsubstituted —N(heteroarylalkyl)-.

In another embodiment of the compounds of formula (IC-1), R¹ is dialkylaminoethyl and A is substituted or unsubstituted —N(heteroarylalkyl)-.

In another embodiment of the compounds of formula (IC-1), R¹ is substituted or unsubstituted diarylaminoethyl and A is substituted or unsubstituted —N(heteroarylalkyl)-.

In another embodiment of the compounds of formula (IC-1), R¹ is H₂N-ethyl and A is substituted or unsubstituted —N(arylalkyl)-.

In another embodiment of the compounds of formula (IC-1), R¹ is aminopropyl and A is substituted or unsubstituted —N(heteroarylalkyl)-.

In another embodiment of the compounds of formula (IC-1), R¹ is dialkylaminopropyl and A is substituted or unsubstituted —N(heteroarylalkyl)-.

In another embodiment of the compounds of formula (IC-1), R¹ is substituted or unsubstituted diarylaminopropyl and A is substituted or unsubstituted —N(heteroarylalkyl)-.

In another embodiment of the compounds of formula (IC-1), R¹ is H₂N-propyl and A is substituted or unsubstituted —N(heteroarylalkyl)-.

In another embodiment of the compounds of formula (IC-1), L³ is —C(R⁴)₂— and A is —O—.

In another embodiment of the compounds of formula (IC-1), L³ is —C(O)— and A is —O—.

In another embodiment of the compounds of formula (IC-1), L³ is —C(R⁴)₂— and A is —NH—.

In another embodiment of the compounds of formula (IC-1), L³ is —C(O)— and A is —NH—.

In another embodiment of the compounds of formula (IC-1), L³ is —C(R⁴)₂— and A is —N(alkyl)-.

In another embodiment of the compounds of formula (IC-1), L³ is —C(O)— and A is —N(alkyl)-.

In another embodiment of the compounds of formula (IC-1), L³ is —C(R⁴)₂— and A is —N(acyl)-.

In another embodiment of the compounds of formula (IC-1), L³ is —C(O)— and A is —N(acyl)-.

In another embodiment of the compounds of formula (IC-1), L³ is —C(R⁴)₂— and A is substituted or unsubstituted —N(arylalkyl)-.

In another embodiment of the compounds of formula (IC-1), L³ is —C(O)— and A is substituted or unsubstituted —N(arylalkyl)-.

In another embodiment of the compounds of formula (IC-1), L³ is —C(R⁴)₂— and A is substituted or unsubstituted —N(heteroarylalkyl)-.

In another embodiment of the compounds of formula (IC-1), L³ is —C(O)— and A is substituted or unsubstituted —N(heteroarylalkyl)-.

In another embodiment of the compounds of formula (IC-1), L³ is —C(R⁴)₂—, A is —O—, and R¹ is H.

In another embodiment of the compounds of formula (IC-1), L³ is —C(O)—, A is —O—, and R¹ is H.

In another embodiment of the compounds of formula (IC-1), L³ is —C(R⁴)₂—, A is —NH—, and R¹ is H.

In another embodiment of the compounds of formula (IC-1), L³ is —C(O)—, A is —NH—, and R¹ is H.

In another embodiment of the compounds of formula (IC-1), L³ is —C(R⁴)₂—, A is —N(alkyl)-, and R¹ is H.

In another embodiment of the compounds of formula (IC-1), L³ is —C(O)—, A is —N(alkyl)-, and R¹ is H.

In another embodiment of the compounds of formula (IC-1), L³ is —C(R⁴)₂—, A is —N(acyl)-, and R¹ is H.

In another embodiment of the compounds of formula (IC-1), L³ is —C(O)—, A is —N(acyl)-, and R¹ is H.

In another embodiment of the compounds of formula (IC-1), L³ is —C(R⁴)₂—, A is substituted or unsubstituted —N(arylalkyl)-, and R¹ is H.

In another embodiment of the compounds of formula (IC-1), L³ is —C(O)—, A is substituted or unsubstituted —N(arylalkyl)-, and R¹ is H.

In another embodiment of the compounds of formula (IC-1), L³ is —C(R⁴)₂—, A is substituted or unsubstituted —N(heteroarylalkyl)-, and R¹ is H.

In another embodiment of the compounds of formula (IC-1), L³ is —C(O)—, A is substituted or unsubstituted —N(heteroarylalkyl)-, and R¹ is H.

In another embodiment of the compounds of formula (IC-1), L³ is —C(R⁴)₂—, A is —O—, and R¹ is substituted or unsubstituted arylalkyl.

In another embodiment of the compounds of formula (IC-1), L³ is —C(O)—, A is —O—, and R¹ is substituted or unsubstituted arylalkyl.

In another embodiment of the compounds of formula (IC-1), L³ is —C(R⁴)₂—, A is —NH—, and R¹ is substituted or unsubstituted arylalkyl.

In another embodiment of the compounds of formula (IC-1), L³ is —C(O)—, A is —NH—, and R¹ is substituted or unsubstituted arylalkyl.

In another embodiment of the compounds of formula (IC-1), L³ is —C(R⁴)₂—, A is —N(alkyl)-, and R¹ is substituted or unsubstituted arylalkyl.

In another embodiment of the compounds of formula (IC-1), L³ is —C(O)—, A is —N(alkyl)-, and R¹ is substituted or unsubstituted arylalkyl.

In another embodiment of the compounds of formula (IC-1), L³ is —C(R⁴)₂—, A is —N(acyl)-, and R¹ is substituted or unsubstituted arylalkyl.

In another embodiment of the compounds of formula (IC-1), L³ is —C(O)—, A is —N(acyl)-, and R¹ is substituted or unsubstituted arylalkyl

In another embodiment of the compounds of formula (IC-1), L³ is —C(R⁴)₂—, A is substituted or unsubstituted —N(arylalkyl)-, and R¹ is substituted or unsubstituted arylalkyl.

In another embodiment of the compounds of formula (IC-1), L³ is —C(O)—, A is substituted or unsubstituted —N(arylalkyl)-, and R¹ is substituted or unsubstituted arylalkyl.

In another embodiment of the compounds of formula (IC-1), L³ is —C(R⁴)₂—, A is substituted or unsubstituted —N(heteroarylalkyl)-, and R¹ is substituted or unsubstituted arylalkyl.

In another embodiment of the compounds of formula (IC-1), L³ is —C(O)—, A is substituted or unsubstituted —N(heteroarylalkyl)-, and R¹ is substituted or unsubstituted arylalkyl.

In another embodiment of the compounds of formula (IC-1), L³ is —C(R⁴)₂—, A is —O—, and R¹ is substituted or unsubstituted heteroarylalkyl.

In another embodiment of the compounds of formula (IC-1), L³ is —C(O)—, A is —O—, and R¹ is substituted or unsubstituted heteroarylalkyl.

In another embodiment of the compounds of formula (IC-1), L³ is —C(R⁴)₂—, A is —NH—, and R¹ is substituted or unsubstituted heteroarylalkyl.

In another embodiment of the compounds of formula (IC-1), L³ is —C(O)—, A is —NH—, and R¹ is substituted or unsubstituted heteroarylalkyl.

In another embodiment of the compounds of formula (IC-1), L³ is —C(R⁴)₂—, A is —N(alkyl)-, and R¹ is substituted or unsubstituted heteroarylalkyl.

In another embodiment of the compounds of formula (IC-1), L³ is —C(O)—, A is —N(alkyl)-, and R¹ is substituted or unsubstituted heteroarylalkyl.

In another embodiment of the compounds of formula (IC-1), L³ is —C(R⁴)₂—, A is —N(acyl)-, and R¹ is substituted or unsubstituted heteroarylalkyl.

In another embodiment of the compounds of formula (IC-1), L³ is —C(O)—, A is —N(acyl)-, and R¹ is substituted or unsubstituted heteroarylalkyl

In another embodiment of the compounds of formula (IC-1), L³ is —C(R⁴)₂—, A is substituted or unsubstituted —N(arylalkyl)-, and R¹ is substituted or unsubstituted heteroarylalkyl.

In another embodiment of the compounds of formula (IC-1), L³ is —C(O)—, A is substituted or unsubstituted —N(arylalkyl)-, and R¹ is substituted or unsubstituted heteroarylalkyl.

In another embodiment of the compounds of formula (IC-1), L³ is —C(R⁴)₂—, A is substituted or unsubstituted —N(heteroarylalkyl)-, and R¹ is substituted or unsubstituted heteroarylalkyl.

In another embodiment of the compounds of formula (IC-1), L³ is —C(O)—, A is substituted or unsubstituted —N(heteroarylalkyl)-, and R¹ is substituted or unsubstituted heteroarylalkyl.

In another embodiment, the compounds of the present invention, or pharmaceutically acceptable salts, solvates, prodrugs, tautomers, or esters thereof, have the structure of formula (IC-2):

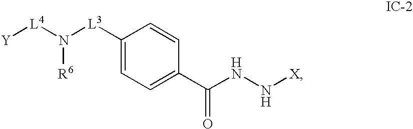

wherein X, Y, L³, L⁴ and R⁶ are as described and defined herein.

In another embodiment of the compounds of formula (IC-2), L³ is —CH₂— or —C(O)—; L⁴ is selected from the group consisting of a covalent bond, —CH₂—, and —C(O)—; X is selected from the group consisting of substituted or unsubstituted phenyl, substituted or unsubstituted quinolyl, substituted or unsubstituted pyrimidyl, substituted or unsubstituted purinyl, substituted or unsubstituted benzimidazolyl, and substituted or unsubstituted pyridyl; Y is selected from the group consisting of substituted or unsubstituted phenyl, substituted or unsubstituted pyridyl, substituted or unsubstituted pyrimidyl, substituted or unsubstituted naphthyl, hydroxyalkyl, alkoxyalkyl, and substituted or unsubstituted naphthylalkylamino; R⁶ is selected from the group consisting of H, alkoxyalkyl, and hydroxyalkyl; or R⁶ and -L⁴-Y, together with the nitrogen atom to which they are both attached, form a substituted or unsubstituted heterocyclyl ring.

In another embodiment of the compounds of formula (IC-2), L⁴ is —C(R⁴)₂—.

In another embodiment of the compounds of formula (IC-2), L⁴ is —CH₂—.

In another embodiment of the compounds of formula (IC-2), L⁴ is —CH(alkyl)-.

In another embodiment of the compounds of formula (IC-2), L³ is —C(R⁴)₂—.

In another embodiment of the compounds of formula (IC-2), L³ is —CH₂—.

In another embodiment of the compounds of formula (IC-2), L³ is —CH(alkyl)-.

In another embodiment of the compounds of formula (IC-2), L³ is —C(O)—.

In another embodiment of the compounds of formula (IC-2), L³ is —S(O)—.

In another embodiment of the compounds of formula (IC-2), L³ is —S(O)₂—.

In another embodiment of the compounds of formula (IC-2), L⁴ is —C(R⁴)₂— and L³ is —C(R⁴)₂—.

In another embodiment of the compounds of formula (IC-2), L⁴ is —CH₂— and L³ is —C(R⁴)₂—.

In another embodiment of the compounds of formula (IC-2), L⁴ is —C(R⁴)₂— and L³ is —CH₂—.

In another embodiment of the compounds of formula (IC-2), L⁴ is —CH₂— and L³ is —CH₂—.

In another embodiment of the compounds of formula (IC-2), L⁴ is —CH(alkyl)- and L³ is —CH₂—.

In another embodiment of the compounds of formula (IC-2), L⁴ is —CH₂— and L³ is —CH(alkyl)-.

In another embodiment of the compounds of formula (IC-2), L⁴ is —CH(alkyl)- and L³ is —CH(alkyl)-.

In another embodiment of the compounds of formula (IC-2), L⁴ is —C(R⁴)₂— and L³ is —C(O)—.

In another embodiment of the compounds of formula (IC-2), L⁴ is —CH₂— and L³ is —C(O)—.

In another embodiment of the compounds of formula (IC-2), L⁴ is —CH(alkyl)- and L³ is —C(O)—.

In another embodiment of the compounds of formula (IC-2), L⁴ is —C(R⁴)₂—, L³ is —C(R⁴)₂—, and R⁶ is H.

In another embodiment of the compounds of formula (IC-2), L⁴ is —CH₂—, L³ is —C(R⁴)₂—, and R⁶ is H.

In another embodiment of the compounds of formula (IC-2), L⁴ is —C(R⁴)₂—, L³ is —CH₂—, and R⁶ is H.

In another embodiment of the compounds of formula (IC-2), L⁴ is —CH₂—, L³ is —CH₂—, and R⁶ is H.

In another embodiment of the compounds of formula (IC-2), L⁴ is —CH(alkyl)-, L³ is —CH₂—, and R⁶ is H.

In another embodiment of the compounds of formula (IC-2), L⁴ is —CH₂—, L³ is —CH(alkyl)-, and R¹ is H.

In another embodiment of the compounds of formula (IC-2), L⁴ is —CH(alkyl)-, L³ is —CH(alkyl)-, and R⁶ is H.

In another embodiment of the compounds of formula (IC-2), L⁴ is —C(R⁴)₂— and L³ is —C(O)—, and R⁶ is H.

In another embodiment of the compounds of formula (IC-2), L⁴ is —CH₂—, L³ is —C(O)—, and R⁶ is H.

In another embodiment of the compounds of formula (IC-2), L⁴ is —CH(alkyl)-, L³ is —C(O)—, and R⁶ is H.

In another embodiment of the compounds of formula (IC-2), L⁴ is —C(R⁴)₂—, L³ is —C(R⁴)₂—, and R⁶ is alkyl.

In another embodiment of the compounds of formula (IC-2), L⁴ is —CH₂—, L³ is —C(R⁴)₂—, and R⁶ is alkyl.

In another embodiment of the compounds of formula (IC-2), L⁴ is —C(R⁴)₂—, L³ is —CH₂—, and R⁶ is alkyl.

In another embodiment of the compounds of formula (IC-2), L⁴ is —CH₂—, L³ is —CH₂—, and R⁶ is alkyl.

In another embodiment of the compounds of formula (IC-2), L⁴ is —CH(alkyl)-, L³ is —CH₂—, and R⁶ is alkyl.

In another embodiment of the compounds of formula (IC-2), L⁴ is —CH₂—, L³ is —CH(alkyl)-, and R⁶ is alkyl.

In another embodiment of the compounds of formula (IC-2), L⁴ is —CH(alkyl)-, L³ is —CH(alkyl)-, and R⁶ is alkyl.

In another embodiment of the compounds of formula (IC-2), L⁴ is —C(R⁴)₂— and L³ is —C(O)—, and R⁶ is alkyl.

In another embodiment of the compounds of formula (IC-2), L⁴ is —CH₂—, L³ is —C(O)—, and R⁶ is alkyl.

In another embodiment of the compounds of formula (IC-2), L⁴ is —CH(alkyl)-, L³ is —C(O)—, and R⁶ is alkyl.

In another embodiment of the compounds of formula (IC-2), L⁴ is —C(R⁴)₂—, L³ is —C(R⁴)₂—, and R⁶ is substituted or unsubstituted cycloalkyl.

In another embodiment of the compounds of formula (IC-2), L⁴ is —CH₂—, L³ is —C(R⁴)₂—, and R⁶ is substituted or unsubstituted cycloalkyl.

In another embodiment of the compounds of formula (IC-2), L⁴ is —C(R⁴)₂—, L³ is —CH₂—, and R⁶ is substituted or unsubstituted cycloalkyl.

In another embodiment of the compounds of formula (IC-2), L⁴ is —CH₂—, L³ is —CH₂—, and R⁶ is substituted or unsubstituted cycloalkyl.

In another embodiment of the compounds of formula (IC-2), L⁴ is —CH(alkyl)-, L³ is —CH₂—, and R⁶ is substituted or unsubstituted cycloalkyl.

In another embodiment of the compounds of formula (IC-2), L⁴ is —CH₂—, L³ is —CH(alkyl)-, and R⁶ is substituted or unsubstituted cycloalkyl.

In another embodiment of the compounds of formula (IC-2), L⁴ is —CH(alkyl)-, L³ is —CH(alkyl)-, and R⁶ is substituted or unsubstituted cycloalkyl.

In another embodiment of the compounds of formula (IC-2), L⁴ is —C(R⁴)₂— and L³ is —C(O)—, and R⁶ is substituted or unsubstituted cycloalkyl.

In another embodiment of the compounds of formula (IC-2), L⁴ is —CH₂—, L³ is —C(O)—, and R⁶ is substituted or unsubstituted cycloalkyl.

In another embodiment of the compounds of formula (IC-2), L⁴ is —CH(alkyl)-, L³ is —C(O)—, and R⁶ is substituted or unsubstituted cycloalkyl.

In another embodiment of the compounds of formula (IC-2), L⁴ is —C(R⁴)₂—, L³ is —C(R⁴)₂—, and R⁶ is substituted or unsubstituted arylalkyl.

In another embodiment of the compounds of formula (IC-2), L⁴ is —CH₂—, L³ is —C(R⁴)₂—, and R⁶ is substituted or unsubstituted arylalkyl.

In another embodiment of the compounds of formula (IC-2), L⁴ is —C(R⁴)₂—, L³ is —CH₂—, and R⁶ is substituted or unsubstituted arylalkyl.

In another embodiment of the compounds of formula (IC-2), L⁴ is —CH₂—, L³ is —CH₂—, and R⁶ is substituted or unsubstituted arylalkyl.

In another embodiment of the compounds of formula (IC-2), L⁴ is —CH(alkyl)-, L³ is —CH₂—, and R⁶ is substituted or unsubstituted arylalkyl.

In another embodiment of the compounds of formula (IC-2), L⁴ is —CH₂—, L³ is —CH(alkyl)-, and R⁶ is substituted or unsubstituted arylalkyl.

In another embodiment of the compounds of formula (IC-2), L⁴ is —CH(alkyl)-, L³ is —CH(alkyl)-, and R⁶ is substituted or unsubstituted arylalkyl.

In another embodiment of the compounds of formula (IC-2), L⁴ is —C(R⁴)₂— and L³ is —C(O)—, and R⁶ is substituted or unsubstituted arylalkyl.

In another embodiment of the compounds of formula (IC-2), L⁴ is —CH₂—, L³ is —C(O)—, and R⁶ is substituted or unsubstituted arylalkyl.

In another embodiment of the compounds of formula (IC-2), L⁴ is —CH(alkyl)-, L³ is —C(O)—, and R¹ is substituted or unsubstituted arylalkyl.

In another embodiment of the compounds of formula (IC-2), L⁴ is —C(R⁴)₂—, L³ is —C(R⁴)₂—, and R⁶ is substituted or unsubstituted benzyl.

In another embodiment of the compounds of formula (IC-2), L⁴ is —CH₂—, L³ is —C(R⁴)₂—, and R⁶ is substituted or unsubstituted benzyl.

In another embodiment of the compounds of formula (IC-2), L⁴ is —C(R⁴)₂—, L³ is —CH₂—, and R⁶ is substituted or unsubstituted benzyl.

In another embodiment of the compounds of formula (IC-2), L⁴ is —CH₂—, L³ is —CH₂—, and R⁶ is substituted or unsubstituted benzyl.

In another embodiment of the compounds of formula (IC-2), L⁴ is —CH(alkyl)-, L³ is —CH₂—, and R⁶ is substituted or unsubstituted benzyl.

In another embodiment of the compounds of formula (IC-2), L⁴ is —CH₂—, L³ is —CH(alkyl)-, and R¹ is substituted or unsubstituted benzyl.

In another embodiment of the compounds of formula (IC-2), L⁴ is —CH(alkyl)-, L³ is —CH(alkyl)-, and R⁶ is substituted or unsubstituted benzyl.

In another embodiment of the compounds of formula (IC-2), L⁴ is —C(R⁴)₂— and L³ is —C(O)—, and R⁶ is substituted or unsubstituted benzyl.

In another embodiment of the compounds of formula (IC-2), L⁴ is —CH₂—, L³ is —C(O)—, and R⁶ is substituted or unsubstituted benzyl.

In another embodiment of the compounds of formula (IC-2), L⁴ is —CH(alkyl)-, L³ is —C(O)—, and R⁶ is substituted or unsubstituted benzyl.

In another embodiment of the compounds of formula (IC-2), L⁴ is —C(R⁴)₂—, L³ is —C(R⁴)₂—, and R⁶ is substituted or unsubstituted heteroarylalkyl.

In another embodiment of the compounds of formula (IC-2), L⁴ is —CH₂—, L³ is —C(R⁴)₂—, and R⁶ is substituted or unsubstituted heteroarylalkyl.

In another embodiment of the compounds of formula (IC-2), L⁴ is —C(R⁴)₂—, L³ is —CH₂—, and R⁶ is substituted or unsubstituted heteroarylalkyl.

In another embodiment of the compounds of formula (IC-2), L⁴ is —CH₂—, L³ is —CH₂—, and R⁶ is substituted or unsubstituted heteroarylalkyl.

In another embodiment of the compounds of formula (IC-2), L⁴ is —CH(alkyl)-, L³ is —CH₂—, and R⁶ is substituted or unsubstituted heteroarylalkyl.

In another embodiment of the compounds of formula (IC-2), L⁴ is —CH₂—, L³ is —CH(alkyl)-, and R⁶ is substituted or unsubstituted heteroarylalkyl.

In another embodiment of the compounds of formula (IC-2), L⁴ is —CH(alkyl)-, L³ is —CH(alkyl)-, and R⁶ is substituted or unsubstituted heteroarylalkyl.

In another embodiment of the compounds of formula (IC-2), L⁴ is —C(R⁴)₂— and L³ is —C(O)—, and R⁶ is substituted or unsubstituted heteroarylalkyl.

In another embodiment of the compounds of formula (IC-2), L⁴ is —CH₂—, L³ is —C(O)—, and R⁶ is substituted or unsubstituted heteroarylalkyl.

In another embodiment of the compounds of formula (IC-2), L⁴ is —CH(alkyl)-, L³ is —C(O)—, and R⁶ is substituted or unsubstituted heteroarylalkyl.

In another embodiment of the compounds of formula (IC-2), L⁴ is —C(R⁴)₂—, L³ is —C(R⁴)₂—, R⁶ is H, and Y is substituted or unsubstituted aryl.

In another embodiment of the compounds of formula (IC-2), L⁴ is —CH₂—, L³ is —C(R⁴)₂—, R⁶ is H, and Y is substituted or unsubstituted aryl.

In another embodiment of the compounds of formula (IC-2), L⁴ is —C(R⁴)₂—, L³ is —CH₂—, R⁶ is H, and Y is substituted or unsubstituted aryl.

In another embodiment of the compounds of formula (IC-2), L⁴ is —CH₂—, L³ is —CH₂—, R⁶ is H, and Y is substituted or unsubstituted aryl.

In another embodiment of the compounds of formula (IC-2), L⁴ is —CH(alkyl)-, L³ is —CH₂—, R⁶ is H, and Y is substituted or unsubstituted aryl.

In another embodiment of the compounds of formula (IC-2), L⁴ is —CH₂—, L³ is —CH(alkyl)-, R⁶ is H, and Y is substituted or unsubstituted aryl.

In another embodiment of the compounds of formula (IC-2), L⁴ is —CH(alkyl)-, L³ is —CH(alkyl)-, R⁶ is H, and Y is substituted or unsubstituted aryl.

In another embodiment of the compounds of formula (IC-2), L⁴ is —C(R⁴)₂— and L³ is —C(O)—, R⁶ is H, and Y is substituted or unsubstituted aryl.

In another embodiment of the compounds of formula (IC-2), L⁴ is —CH₂—, L³ is —C(O)—, R⁶ is H, and Y is substituted or unsubstituted aryl.

In another embodiment of the compounds of formula (IC-2), L⁴ is —CH(alkyl)-, L³ is —C(O)—, R⁶ is H, and Y is substituted or unsubstituted aryl.

In another embodiment of the compounds of formula (IC-2), L⁴ is —C(R⁴)₂—, L³ is —C(R⁴)₂—, R⁶ is alkyl, and Y is substituted or unsubstituted aryl.

In another embodiment of the compounds of formula (IC-2), L⁴ is —CH₂—, L³ is —C(R⁴)₂—, R⁶ is alkyl, and Y is substituted or unsubstituted aryl.

In another embodiment of the compounds of formula (IC-2), L⁴ is —C(R⁴)₂—, L³ is —CH₂—, R⁶ is alkyl, and Y is substituted or unsubstituted aryl.

In another embodiment of the compounds of formula (IC-2), L⁴ is —CH₂—, L³ is —CH₂—, R⁶ is alkyl, and Y is substituted or unsubstituted aryl.

In another embodiment of the compounds of formula (IC-2), L⁴ is —CH(alkyl)-, L³ is —CH₂—, R⁶ is alkyl, and Y is substituted or unsubstituted aryl.

In another embodiment of the compounds of formula (IC-2), L⁴ is —CH₂—, L³ is —CH(alkyl)-, R⁶ is alkyl, and Y is substituted or unsubstituted aryl.

In another embodiment of the compounds of formula (IC-2), L⁴ is —CH(alkyl)-, L³ is —CH(alkyl)-, R⁶ is alkyl, and Y is substituted or unsubstituted aryl.

In another embodiment of the compounds of formula (IC-2), L⁴ is —C(R⁴)₂— and L³ is —C(O)—, R⁶ is alkyl, and Y is substituted or unsubstituted aryl.

In another embodiment of the compounds of formula (IC-2), L⁴ is —CH₂—, L³ is —C(O)—, R⁶ is alkyl, and Y is substituted or unsubstituted aryl.

In another embodiment of the compounds of formula (IC-2), L⁴ is —CH(alkyl)-, L³ is —C(O)—, R⁶ is alkyl, and Y is substituted or unsubstituted aryl.

In another embodiment of the compounds of formula (IC-2), L⁴ is —C(R⁴)₂—, L³ is —C(R⁴)₂—, R⁶ is substituted or unsubstituted cycloalkyl, and Y is substituted or unsubstituted aryl.

In another embodiment of the compounds of formula (IC-2), L⁴ is —CH₂—, L³ is —C(R⁴)₂—, R⁶ is substituted or unsubstituted cycloalkyl, and Y is substituted or unsubstituted aryl.

In another embodiment of the compounds of formula (IC-2), L⁴ is —C(R⁴)₂—, L³ is —CH₂—, R⁶ is substituted or unsubstituted cycloalkyl, and Y is substituted or unsubstituted aryl.

In another embodiment of the compounds of formula (IC-2), L⁴ is —CH₂—, L³ is —CH₂—, R⁶ is substituted or unsubstituted cycloalkyl, and Y is substituted or unsubstituted aryl.

In another embodiment of the compounds of formula (IC-2), L⁴ is —CH(alkyl)-, L³ is —CH₂—, R⁶ is substituted or unsubstituted cycloalkyl, and Y is substituted or unsubstituted aryl.

In another embodiment of the compounds of formula (IC-2), L⁴ is —CH₂—, L³ is —CH(alkyl)-, R⁶ is substituted or unsubstituted cycloalkyl, and Y is substituted or unsubstituted aryl.

In another embodiment of the compounds of formula (IC-2), L⁴ is —CH(alkyl)-, L³ is —CH(alkyl)-, R⁶ is substituted or unsubstituted cycloalkyl, and Y is substituted or unsubstituted aryl.

In another embodiment of the compounds of formula (IC-2), L⁴ is —C(R⁴)₂— and L³ is —C(O)—, R⁶ is substituted or unsubstituted cycloalkyl, and Y is substituted or unsubstituted aryl.

In another embodiment of the compounds of formula (IC-2), L⁴ is —CH₂—, L³ is —C(O)—, R⁶ is substituted or unsubstituted cycloalkyl, and Y is substituted or unsubstituted aryl.

In another embodiment of the compounds of formula (IC-2), L⁴ is —CH(alkyl)-, L³ is —C(O)—, R⁶ is substituted or unsubstituted cycloalkyl, and Y is substituted or unsubstituted aryl.

In another embodiment of the compounds of formula (IC-2), L⁴ is —C(R⁴)₂—, L³ is —C(R⁴)₂—, R⁶ is substituted or unsubstituted arylalkyl, and Y is substituted or unsubstituted aryl.

In another embodiment of the compounds of formula (IC-2), L⁴ is —CH₂—, L³ is —C(R⁴)₂—, R⁶ is substituted or unsubstituted arylalkyl, and Y is substituted or unsubstituted aryl.

In another embodiment of the compounds of formula (IC-2), L⁴ is —C(R⁴)₂—, L³ is —CH₂—, R⁶ is substituted or unsubstituted arylalkyl, and Y is substituted or unsubstituted aryl.

In another embodiment of the compounds of formula (IC-2), L⁴ is —CH₂—, L³ is —CH₂—, R⁶ is substituted or unsubstituted arylalkyl, and Y is substituted or unsubstituted aryl.

In another embodiment of the compounds of formula (IC-2), L⁴ is —CH(alkyl)-, L³ is —CH₂—, R⁶ is substituted or unsubstituted arylalkyl, and Y is substituted or unsubstituted aryl.

In another embodiment of the compounds of formula (IC-2), L⁴ is —CH₂—, L³ is —CH(alkyl)-, R⁶ is substituted or unsubstituted arylalkyl, and Y is substituted or unsubstituted aryl.

In another embodiment of the compounds of formula (IC-2), L⁴ is —CH(alkyl)-, L³ is —CH(alkyl)-, R⁶ is substituted or unsubstituted arylalkyl, and Y is substituted or unsubstituted aryl.

In another embodiment of the compounds of formula (IC-2), L⁴ is —C(R⁴)₂— and L³ is —C(O)—, R⁶ is substituted or unsubstituted arylalkyl, and Y is substituted or unsubstituted aryl.

In another embodiment of the compounds of formula (IC-2), L⁴ is —CH₂—, L³ is —C(O)—, R⁶ is substituted or unsubstituted arylalkyl, and Y is substituted or unsubstituted aryl.

In another embodiment of the compounds of formula (IC-2), L⁴ is —CH(alkyl)-, L³ is —C(O)—, R⁶ is substituted or unsubstituted arylalkyl, and Y is substituted or unsubstituted aryl.

In another embodiment of the compounds of formula (IC-2), L⁴ is —C(R⁴)₂—, L³ is —C(R⁴)₂—, R⁶ is substituted or unsubstituted benzyl, and Y is substituted or unsubstituted aryl.

In another embodiment of the compounds of formula (IC-2), L⁴ is —CH₂—, L³ is —C(R⁴)₂—, R⁶ is substituted or unsubstituted benzyl, and Y is substituted or unsubstituted aryl.

In another embodiment of the compounds of formula (IC-2), L⁴ is —C(R⁴)₂—, L³ is —CH₂—, R⁶ is substituted or unsubstituted benzyl, and Y is substituted or unsubstituted aryl.

In another embodiment of the compounds of formula (IC-2), L⁴ is —CH₂—, L³ is —CH₂—, R⁶ is substituted or unsubstituted benzyl, and Y is substituted or unsubstituted aryl.

In another embodiment of the compounds of formula (IC-2), L⁴ is —CH(alkyl)-, L³ is —CH₂—, R⁶ is substituted or unsubstituted benzyl, and Y is substituted or unsubstituted aryl.

In another embodiment of the compounds of formula (IC-2), L⁴ is —CH₂—, L³ is —CH(alkyl)-, R⁶ is substituted or unsubstituted benzyl, and Y is substituted or unsubstituted aryl.

In another embodiment of the compounds of formula (IC-2), L⁴ is —CH(alkyl)-, L³ is —CH(alkyl)-, R⁶ is substituted or unsubstituted benzyl, and Y is substituted or unsubstituted aryl.

In another embodiment of the compounds of formula (IC-2), L⁴ is —C(R⁴)₂— and L³ is —C(O)—, R⁶ is substituted or unsubstituted benzyl, and Y is substituted or unsubstituted aryl.

In another embodiment of the compounds of formula (IC-2), L⁴ is —CH₂—, L³ is —C(O)—, R⁶ is substituted or unsubstituted benzyl, and Y is substituted or unsubstituted aryl.

In another embodiment of the compounds of formula (IC-2), L⁴ is —CH(alkyl)-, L³ is —C(O)—, R⁶ is substituted or unsubstituted benzyl, and Y is substituted or unsubstituted aryl.

In another embodiment of the compounds of formula (IC-2), L⁴ is —C(R⁴)₂—, L³ is —C(R⁴)₂—, R⁶ is substituted or unsubstituted heteroarylalkyl, and Y is substituted or unsubstituted aryl.

In another embodiment of the compounds of formula (IC-2), L⁴ is —CH₂—, L³ is —C(R⁴)₂—, R⁶ is substituted or unsubstituted heteroarylalkyl, and Y is substituted or unsubstituted aryl.

In another embodiment of the compounds of formula (IC-2), L⁴ is —C(R⁴)₂—, L³ is —CH₂—, R⁶ is substituted or unsubstituted heteroarylalkyl, and Y is substituted or unsubstituted aryl.

In another embodiment of the compounds of formula (IC-2), L⁴ is —CH₂—, L³ is —CH₂—, R⁶ is substituted or unsubstituted heteroarylalkyl, and Y is substituted or unsubstituted aryl.

In another embodiment of the compounds of formula (IC-2), L⁴ is —CH(alkyl)-, L³ is —CH₂—, R⁶ is substituted or unsubstituted heteroarylalkyl, and Y is substituted or unsubstituted aryl.

In another embodiment of the compounds of formula (IC-2), L⁴ is —CH₂—, L³ is —CH(alkyl)-, R⁶ is substituted or unsubstituted heteroarylalkyl, and Y is substituted or unsubstituted aryl.

In another embodiment of the compounds of formula (IC-2), L⁴ is —CH(alkyl)-, L³ is —CH(alkyl)-, R⁶ is substituted or unsubstituted heteroarylalkyl, and Y is substituted or unsubstituted aryl.

In another embodiment of the compounds of formula (IC-2), L⁴ is —C(R⁴)₂— and L³ is —C(O)—, R⁶ is substituted or unsubstituted heteroarylalkyl, and Y is substituted or unsubstituted aryl.

In another embodiment of the compounds of formula (IC-2), L⁴ is —CH₂—, L³ is —C(O)—, R⁶ is substituted or unsubstituted heteroarylalkyl, and Y is substituted or unsubstituted aryl.

In another embodiment of the compounds of formula (IC-2), L⁴ is —CH(alkyl)-, L³ is —C(O)—, R⁶ is substituted or unsubstituted heteroarylalkyl, and Y is substituted or unsubstituted aryl.

In another embodiment of the compounds of formula (IC-2), L⁴ is —C(R⁴)₂—, L³ is —C(R⁴)₂—, R⁶ is H, and Y is substituted or unsubstituted heteroaryl.

In another embodiment of the compounds of formula (IC-2), L⁴ is —CH₂—, L³ is —C(R⁴)₂—, R⁶ is H, and Y is substituted or unsubstituted heteroaryl.

In another embodiment of the compounds of formula (IC-2), L⁴ is —C(R⁴)₂—, L³ is —CH₂—, R⁶ is H, and Y is substituted or unsubstituted heteroaryl.

In another embodiment of the compounds of formula (IC-2), L⁴ is —CH₂—, L³ is —CH₂—, R⁶ is H, and Y is substituted or unsubstituted heteroaryl.

In another embodiment of the compounds of formula (IC-2), L⁴ is —CH(alkyl)-, L³ is —CH₂—, R⁶ is H, and Y is substituted or unsubstituted heteroaryl.

In another embodiment of the compounds of formula (IC-2), L⁴ is —CH₂—, L³ is —CH(alkyl)-, R⁶ is H, and Y is substituted or unsubstituted heteroaryl.

In another embodiment of the compounds of formula (IC-2), L⁴ is —CH(alkyl)-, L³ is —CH(alkyl)-, R⁶ is H, and Y is substituted or unsubstituted heteroaryl.

In another embodiment of the compounds of formula (IC-2), L⁴ is —C(R⁴)₂— and L³ is —C(O)—, R⁶ is H, and Y is substituted or unsubstituted heteroaryl.

In another embodiment of the compounds of formula (IC-2), L⁴ is —CH₂—, L³ is —C(O)—, R⁶ is H, and Y is substituted or unsubstituted heteroaryl.

In another embodiment of the compounds of formula (IC-2), L⁴ is —CH(alkyl)-, L³ is —C(O)—, R⁶ is H, and Y is substituted or unsubstituted heteroaryl.

In another embodiment of the compounds of formula (IC-2), L⁴ is —C(R⁴)₂—, L³ is —C(R⁴)₂—, R⁶ is alkyl, and Y is substituted or unsubstituted heteroaryl.

In another embodiment of the compounds of formula (IC-2), L⁴ is —CH₂—, L³ is —C(R⁴)₂—, R⁶ is alkyl, and Y is substituted or unsubstituted heteroaryl.

In another embodiment of the compounds of formula (IC-2), L⁴ is —C(R⁴)₂—, L³ is —CH₂—, R⁶ is alkyl, and Y is substituted or unsubstituted heteroaryl.

In another embodiment of the compounds of formula (IC-2), L⁴ is —CH₂—, L³ is —CH₂—, R⁶ is alkyl, and Y is substituted or unsubstituted heteroaryl.

In another embodiment of the compounds of formula (IC-2), L⁴ is —CH(alkyl)-, L³ is —CH₂—, R⁶ is alkyl, and Y is substituted or unsubstituted heteroaryl.

In another embodiment of the compounds of formula (IC-2), L⁴ is —CH₂—, L³ is —CH(alkyl)-, R⁶ is alkyl, and Y is substituted or unsubstituted heteroaryl.

In another embodiment of the compounds of formula (IC-2), L⁴ is —CH(alkyl)-, L³ is —CH(alkyl)-, R⁶ is alkyl, and Y is substituted or unsubstituted heteroaryl.

In another embodiment of the compounds of formula (IC-2), L⁴ is —C(R⁴)₂— and L³ is —C(O)—, R⁶ is alkyl, and Y is substituted or unsubstituted heteroaryl.

In another embodiment of the compounds of formula (IC-2), L⁴ is —CH₂—, L³ is —C(O)—, R⁶ is alkyl, and Y is substituted or unsubstituted heteroaryl.

In another embodiment of the compounds of formula (IC-2), L⁴ is —CH(alkyl)-, L³ is —C(O)—, R⁶ is alkyl, and Y is substituted or unsubstituted heteroaryl.

In another embodiment of the compounds of formula (IC-2), L⁴ is —C(R⁴)₂—, L³ is —C(R⁴)₂—, R⁶ is substituted or unsubstituted cycloalkyl, and Y is substituted or unsubstituted heteroaryl.

In another embodiment of the compounds of formula (IC-2), L⁴ is —CH₂—, L³ is —C(R⁴)₂—, R⁶ is substituted or unsubstituted cycloalkyl, and Y is substituted or unsubstituted heteroaryl.

In another embodiment of the compounds of formula (IC-2), L⁴ is —C(R⁴)₂—, L³ is —CH₂—, R⁶ is substituted or unsubstituted cycloalkyl, and Y is substituted or unsubstituted heteroaryl.

In another embodiment of the compounds of formula (IC-2), L⁴ is —CH₂—, L³ is —CH₂—, R⁶ is substituted or unsubstituted cycloalkyl, and Y is substituted or unsubstituted heteroaryl.

In another embodiment of the compounds of formula (IC-2), L⁴ is —CH(alkyl)-, L³ is —CH₂—, R⁶ is substituted or unsubstituted cycloalkyl, and Y is substituted or unsubstituted heteroaryl.

In another embodiment of the compounds of formula (IC-2), L⁴ is —CH₂—, L³ is —CH(alkyl)-, R⁶ is substituted or unsubstituted cycloalkyl, and Y is substituted or unsubstituted heteroaryl.

In another embodiment of the compounds of formula (IC-2), L⁴ is —CH(alkyl)-, L³ is —CH(alkyl)-, R⁶ is substituted or unsubstituted cycloalkyl, and Y is substituted or unsubstituted heteroaryl.

In another embodiment of the compounds of formula (IC-2), L⁴ is —C(R⁴)₂— and L³ is —C(O)—, R⁶ is substituted or unsubstituted cycloalkyl, and Y is substituted or unsubstituted heteroaryl.

In another embodiment of the compounds of formula (IC-2), L⁴ is —CH₂—, L³ is —C(O)—, R⁶ is substituted or unsubstituted cycloalkyl, and Y is substituted or unsubstituted heteroaryl.

In another embodiment of the compounds of formula (IC-2), L⁴ is —CH(alkyl)-, L³ is —C(O)—, R⁶ is substituted or unsubstituted cycloalkyl, and Y is substituted or unsubstituted heteroaryl.

In another embodiment of the compounds of formula (IC-2), L⁴ is —C(R⁴)₂—, L³ is —C(R⁴)₂—, R⁶ is substituted or unsubstituted arylalkyl, and Y is substituted or unsubstituted heteroaryl.

In another embodiment of the compounds of formula (IC-2), L⁴ is —CH₂—, L³ is —C(R⁴)₂—, R⁶ is substituted or unsubstituted arylalkyl, and Y is substituted or unsubstituted heteroaryl.

In another embodiment of the compounds of formula (IC-2), L⁴ is —C(R⁴)₂—, L³ is —CH₂—, R⁶ is substituted or unsubstituted arylalkyl, and Y is substituted or unsubstituted heteroaryl.

In another embodiment of the compounds of formula (IC-2), L⁴ is —CH₂—, L³ is —CH₂—, R⁶ is substituted or unsubstituted arylalkyl, and Y is substituted or unsubstituted heteroaryl.

In another embodiment of the compounds of formula (IC-2), L⁴ is —CH(alkyl)-, L³ is —CH₂—, R⁶ is substituted or unsubstituted arylalkyl, and Y is substituted or unsubstituted heteroaryl.

In another embodiment of the compounds of formula (IC-2), L⁴ is —CH₂—, L³ is —CH(alkyl)-, R⁶ is substituted or unsubstituted arylalkyl, and Y is substituted or unsubstituted heteroaryl.

In another embodiment of the compounds of formula (IC-2), L⁴ is —CH(alkyl)-, L³ is —CH(alkyl)-, R⁶ is substituted or unsubstituted arylalkyl, and Y is substituted or unsubstituted heteroaryl.

In another embodiment of the compounds of formula (IC-2), L⁴ is —C(R⁴)₂— and L³ is —C(O)—, R⁶ is substituted or unsubstituted arylalkyl, and Y is substituted or unsubstituted heteroaryl.

In another embodiment of the compounds of formula (IC-2), L⁴ is —CH₂—, L³ is —C(O)—, R⁶ is substituted or unsubstituted arylalkyl, and Y is substituted or unsubstituted heteroaryl.

In another embodiment of the compounds of formula (IC-2), L⁴ is —CH(alkyl)-, L³ is —C(O)—, R⁶ is substituted or unsubstituted arylalkyl, and Y is substituted or unsubstituted heteroaryl.

In another embodiment of the compounds of formula (IC-2), L⁴ is —C(R⁴)₂—, L³ is —C(R⁴)₂—, R⁶ is substituted or unsubstituted benzyl, and Y is substituted or unsubstituted heteroaryl.

In another embodiment of the compounds of formula (IC-2), L⁴ is —CH₂—, L³ is —C(R⁴)₂—, R⁶ is substituted or unsubstituted benzyl, and Y is substituted or unsubstituted heteroaryl.

In another embodiment of the compounds of formula (IC-2), L⁴ is —C(R⁴)₂—, L³ is —CH₂—, R⁶ is substituted or unsubstituted benzyl, and Y is substituted or unsubstituted heteroaryl.

In another embodiment of the compounds of formula (IC-2), L⁴ is —C₁H₂—, L³ is —CH₂—, R⁶ is substituted or unsubstituted benzyl, and Y is substituted or unsubstituted heteroaryl.

In another embodiment of the compounds of formula (IC-2), L⁴ is —CH(alkyl)-, L³ is —CH₂—, R⁶ is substituted or unsubstituted benzyl, and Y is substituted or unsubstituted heteroaryl.

In another embodiment of the compounds of formula (IC-2), L⁴ is —CH₂—, L³ is —CH(alkyl)-, R⁶ is substituted or unsubstituted benzyl, and Y is substituted or unsubstituted heteroaryl.

In another embodiment of the compounds of formula (IC-2), L⁴ is —CH(alkyl)-, L³ is —CH(alkyl)-, R⁶ is substituted or unsubstituted benzyl, and Y is substituted or unsubstituted heteroaryl.

In another embodiment of the compounds of formula (IC-2), L⁴ is —C(R⁴)₂— and L³ is —C(O)—, R⁶ is substituted or unsubstituted benzyl, and Y is substituted or unsubstituted heteroaryl.

In another embodiment of the compounds of formula (IC-2), L⁴ is —CH₂—, L³ is —C(O)—, R⁶ is substituted or unsubstituted benzyl, and Y is substituted or unsubstituted heteroaryl.

In another embodiment of the compounds of formula (IC-2), L⁴ is —CH(alkyl)-, L³ is —C(O)—, R⁶ is substituted or unsubstituted benzyl, and Y is substituted or unsubstituted heteroaryl.

In another embodiment of the compounds of formula (IC-2), L⁴ is —C(R⁴)₂—, L³ is —C(R⁴)₂—, R⁶ is substituted or unsubstituted heteroarylalkyl, and Y is substituted or unsubstituted heteroaryl.

In another embodiment of the compounds of formula (IC-2), L⁴ is —CH₂—, L³ is —C(R⁴)₂—, R⁶ is substituted or unsubstituted heteroarylalkyl, and Y is substituted or unsubstituted heteroaryl.

In another embodiment of the compounds of formula (IC-2), L⁴ is —C(R⁴)₂—, L³ is —CH₂—, R⁶ is substituted or unsubstituted heteroarylalkyl, and Y is substituted or unsubstituted heteroaryl.

In another embodiment of the compounds of formula (IC-2), L⁴ is —CH₂—, L³ is —CH₂—, R⁶ is substituted or unsubstituted heteroarylalkyl, and Y is substituted or unsubstituted heteroaryl.

In another embodiment of the compounds of formula (IC-2), L⁴ is —CH(alkyl)-, L³ is —CH₂—, R⁶ is substituted or unsubstituted heteroarylalkyl, and Y is substituted or unsubstituted heteroaryl.

In another embodiment of the compounds of formula (IC-2), L⁴ is —CH₂—, L³ is —CH(alkyl)-, R⁶ is substituted or unsubstituted heteroarylalkyl, and Y is substituted or unsubstituted heteroaryl.

In another embodiment of the compounds of formula (IC-2), L⁴ is —CH(alkyl)-, L³ is —CH(alkyl)-, R⁶ is substituted or unsubstituted heteroarylalkyl, and Y is substituted or unsubstituted heteroaryl.

In another embodiment of the compounds of formula (IC-2), L⁴ is —C(R⁴)₂— and L³ is —C(O)—, R⁶ is substituted or unsubstituted heteroarylalkyl, and Y is substituted or unsubstituted heteroaryl.

In another embodiment of the compounds of formula (IC-2), L⁴ is —CH₂—, L³ is —C(O)—, R⁶ is substituted or unsubstituted heteroarylalkyl, and Y is substituted or unsubstituted heteroaryl.

In another embodiment of the compounds of formula (IC-2), L⁴ is —CH(alkyl)-, L³ is —C(O)—, R⁶ is substituted or unsubstituted heteroarylalkyl, and Y is substituted or unsubstituted heteroaryl.

In another embodiment of the compounds of formula (IC-2), X is substituted or unsubstituted aryl.

In another embodiment of the compounds of formula (IC-2), X is substituted or unsubstituted phenyl.

In another embodiment of the compounds of formula (IC-2), X is substituted or unsubstituted heteroaryl.

In another embodiment of the compounds of formula (IC-2), X is substituted or unsubstituted pyridyl.

In another embodiment of the compounds of formula (IC-2), X is substituted or unsubstituted pyrimidyl.

In another embodiment of the compounds of formula (IC-2), X is substituted or unsubstituted benzimidazolyl.

In another embodiment of the compounds of formula (IC-2), X is substituted or unsubstituted thienyl.

In another embodiment of the compounds of formula (IC-2), X is substituted or unsubstituted quinolyl.

In another embodiment of the compounds of formula (IC-2), X is substituted or unsubstituted purinyl.

In another embodiment of the compounds of formula (IC-2), X is substituted or unsubstituted indolyl.

In another embodiment of the compounds of formula (IC-2), L⁴ is —C(R⁴)₂—, L³ is —C(R⁴)₂—, R⁶ is H, Y is substituted or unsubstituted aryl, and X is substituted or unsubstituted aryl.

In another embodiment of the compounds of formula (IC-2), L⁴ is —CH₂—, L³ is —C(R⁴)₂—, R⁶ is H, Y is substituted or unsubstituted aryl, and X is substituted or unsubstituted aryl.

In another embodiment of the compounds of formula (IC-2), L⁴ is —C(R⁴)₂—, L³ is —CH₂—, R⁶ is H, Y is substituted or unsubstituted aryl, and X is substituted or unsubstituted aryl.

In another embodiment of the compounds of formula (IC-2), L⁴ is —CH₂—, L³ is —CH₂—, R⁶ is H, Y is substituted or unsubstituted aryl, and X is substituted or unsubstituted aryl.

In another embodiment of the compounds of formula (IC-2), L⁴ is —CH(alkyl)-, L³ is —CH₂—, R⁶ is H, Y is substituted or unsubstituted aryl, and X is substituted or unsubstituted aryl.

In another embodiment of the compounds of formula (IC-2), L⁴ is —CH₂—, L³ is —CH(alkyl)-, R⁶ is H, Y is substituted or unsubstituted aryl, and X is substituted or unsubstituted aryl.

In another embodiment of the compounds of formula (IC-2), L⁴ is —CH(alkyl)-, L³ is —CH(alkyl)-, R⁶ is H, Y is substituted or unsubstituted aryl, and X is substituted or unsubstituted aryl.

In another embodiment of the compounds of formula (IC-2), L⁴ is —C(R⁴)₂— and L³ is —C(O)—, R⁶ is H, Y is substituted or unsubstituted aryl, and X is substituted or unsubstituted aryl.

In another embodiment of the compounds of formula (IC-2), L⁴ is —CH₂—, L³ is —C(O)—, R⁶ is H, Y is substituted or unsubstituted aryl, and X is substituted or unsubstituted aryl.

In another embodiment of the compounds of formula (IC-2), L⁴ is —CH(alkyl)-, L³ is —C(O)—, R⁶ is H, Y is substituted or unsubstituted aryl, and X is substituted or unsubstituted aryl.

In another embodiment of the compounds of formula (IC-2), L⁴ is —C(R⁴)₂—, L³ is —C(R⁴)₂—, R⁶ is alkyl, Y is substituted or unsubstituted aryl, and X is substituted or unsubstituted aryl.

In another embodiment of the compounds of formula (IC-2), L⁴ is —CH₂—, L³ is —C(R⁴)₂—, R⁶ is alkyl, Y is substituted or unsubstituted aryl, and X is substituted or unsubstituted aryl.

In another embodiment of the compounds of formula (IC-2), L⁴ is —C(R⁴)₂—, L³ is —CH₂—, R⁶ is alkyl, Y is substituted or unsubstituted aryl, and X is substituted or unsubstituted aryl.

In another embodiment of the compounds of formula (IC-2), L⁴ is —CH₂—, L³ is —CH₂—, R⁶ is alkyl, Y is substituted or unsubstituted aryl, and X is substituted or unsubstituted aryl.

In another embodiment of the compounds of formula (IC-2), L⁴ is —CH(alkyl)-, L³ is —CH₂—, R⁶ is alkyl, Y is substituted or unsubstituted aryl, and X is substituted or unsubstituted aryl.

In another embodiment of the compounds of formula (IC-2), L⁴ is —CH₂—, L³ is —CH(alkyl)-, R⁶ is alkyl, Y is substituted or unsubstituted aryl, and X is substituted or unsubstituted aryl.

In another embodiment of the compounds of formula (IC-2), L⁴ is —CH(alkyl)-, L³ is —CH(alkyl)-, R⁶ is alkyl, Y is substituted or unsubstituted aryl, and X is substituted or unsubstituted aryl.

In another embodiment of the compounds of formula (IC-2), L⁴ is —C(R⁴)₂— and L³ is —C(O)—, R⁶ is alkyl, Y is substituted or unsubstituted aryl, and X is substituted or unsubstituted aryl.

In another embodiment of the compounds of formula (IC-2), L⁴ is —CH₂—, L³ is —C(O)—, R⁶ is alkyl, Y is substituted or unsubstituted aryl, and X is substituted or unsubstituted aryl.

In another embodiment of the compounds of formula (IC-2), L⁴ is —CH(alkyl)-, L³ is —C(O)—, R⁶ is alkyl, Y is substituted or unsubstituted aryl, and X is substituted or unsubstituted aryl.

In another embodiment of the compounds of formula (IC-2), L⁴ is —C(R⁴)₂—, L³ is —C(R⁴)₂—, R⁶ is substituted or unsubstituted cycloalkyl, Y is substituted or unsubstituted aryl, and X is substituted or unsubstituted aryl.

In another embodiment of the compounds of formula (IC-2), L⁴ is —CH₂—, L³ is —C(R⁴)₂—, R⁶ is substituted or unsubstituted cycloalkyl, Y is substituted or unsubstituted aryl, and X is substituted or unsubstituted aryl.

In another embodiment of the compounds of formula (IC-2), L⁴ is —C(R⁴)₂—, L³ is —CH₂—, R⁶ is substituted or unsubstituted cycloalkyl, Y is substituted or unsubstituted aryl, and X is substituted or unsubstituted aryl.

In another embodiment of the compounds of formula (IC-2), L⁴ is —CH₂—, L³ is —CH₂—, R⁶ is substituted or unsubstituted cycloalkyl, Y is substituted or unsubstituted aryl, and X is substituted or unsubstituted aryl.

In another embodiment of the compounds of formula (IC-2), L⁴ is —CH(alkyl)-, L³ is —CH₂—, R⁶ is substituted or unsubstituted cycloalkyl, Y is substituted or unsubstituted aryl, and X is substituted or unsubstituted aryl.

In another embodiment of the compounds of formula (IC-2), L⁴ is —CH₂—, L³ is —CH(alkyl)-, R⁶ is substituted or unsubstituted cycloalkyl, Y is substituted or unsubstituted aryl, and X is substituted or unsubstituted aryl.

In another embodiment of the compounds of formula (IC-2), L⁴ is —CH(alkyl)-, L³ is —CH(alkyl)-, R⁶ is substituted or unsubstituted cycloalkyl, Y is substituted or unsubstituted aryl, and X is substituted or unsubstituted aryl.

In another embodiment of the compounds of formula (IC-2), L⁴ is —C(R⁴)₂— and L³ is —C(O)—, R⁶ is substituted or unsubstituted cycloalkyl, Y is substituted or unsubstituted aryl, and X is substituted or unsubstituted aryl.

In another embodiment of the compounds of formula (IC-2), L⁴ is —CH₂—, L³ is —C(O)—, R⁶ is substituted or unsubstituted cycloalkyl, Y is substituted or unsubstituted aryl, and X is substituted or unsubstituted aryl.

In another embodiment of the compounds of formula (IC-2), L⁴ is —CH(alkyl)-, L³ is —C(O)—, R⁶ is substituted or unsubstituted cycloalkyl, Y is substituted or unsubstituted aryl, and X is substituted or unsubstituted aryl.

In another embodiment of the compounds of formula (IC-2), L⁴ is —C(R⁴)₂—, L³ is —C(R⁴)₂—, R⁶ is substituted or unsubstituted arylalkyl, Y is substituted or unsubstituted aryl, and X is substituted or unsubstituted aryl.

In another embodiment of the compounds of formula (IC-2), L⁴ is —CH₂—, L³ is —C(R⁴)₂—, R⁶ is substituted or unsubstituted arylalkyl, Y is substituted or unsubstituted aryl, and X is substituted or unsubstituted aryl.

In another embodiment of the compounds of formula (IC-2), L⁴ is —C(R⁴)₂—, L³ is —CH₂—, R⁶ is substituted or unsubstituted arylalkyl, Y is substituted or unsubstituted aryl, and X is substituted or unsubstituted aryl.

In another embodiment of the compounds of formula (IC-2), L⁴ is —CH₂—, L³ is —CH₂—, R⁶ is substituted or unsubstituted arylalkyl, Y is substituted or unsubstituted aryl, and X is substituted or unsubstituted aryl.

In another embodiment of the compounds of formula (IC-2), L⁴ is —CH(alkyl)-, L³ is —CH₂—, R⁶ is substituted or unsubstituted arylalkyl, Y is substituted or unsubstituted aryl, and X is substituted or unsubstituted aryl.

In another embodiment of the compounds of formula (IC-2), L⁴ is —CH₂—, L³ is —CH(alkyl)-, R⁶ is substituted or unsubstituted arylalkyl, Y is substituted or unsubstituted aryl, and X is substituted or unsubstituted aryl.

In another embodiment of the compounds of formula (IC-2), L⁴ is —CH(alkyl)-, L³ is —CH(alkyl)-, R⁶ is substituted or unsubstituted arylalkyl, Y is substituted or unsubstituted aryl, and X is substituted or unsubstituted aryl.

In another embodiment of the compounds of formula (IC-2), L⁴ is —C(R⁴)₂— and L³ is —C(O)—, R⁶ is substituted or unsubstituted arylalkyl, Y is substituted or unsubstituted aryl, and X is substituted or unsubstituted aryl.

In another embodiment of the compounds of formula (IC-2), L⁴ is —CH₂—, L³ is —C(O)—, R⁶ is substituted or unsubstituted arylalkyl, Y is substituted or unsubstituted aryl, and X is substituted or unsubstituted aryl.

In another embodiment of the compounds of formula (IC-2), L⁴ is —CH(alkyl)-, L³ is —C(O)—, R⁶ is substituted or unsubstituted arylalkyl, Y is substituted or unsubstituted aryl, and X is substituted or unsubstituted aryl.

In another embodiment of the compounds of formula (IC-2), L⁴ is —C(R⁴)₂—, L³ is —C(R⁴)₂—, R⁶ is substituted or unsubstituted benzyl, Y is substituted or unsubstituted aryl, and X is substituted or unsubstituted aryl.

In another embodiment of the compounds of formula (IC-2), L⁴ is —CH₂—, L³ is —C(R⁴)₂—, R⁶ is substituted or unsubstituted benzyl, and Y is substituted or unsubstituted aryl, and X is substituted or unsubstituted aryl.

In another embodiment of the compounds of formula (IC-2), L⁴ is —C(R⁴)₂—, L³ is —CH₂—, R⁶ is substituted or unsubstituted benzyl, Y is substituted or unsubstituted aryl, and X is substituted or unsubstituted aryl.

In another embodiment of the compounds of formula (IC-2), L⁴ is —CH₂—, L³ is —CH₂—, R⁶ is substituted or unsubstituted benzyl, Y is substituted or unsubstituted aryl, and X is substituted or unsubstituted aryl.

In another embodiment of the compounds of formula (IC-2), L⁴ is —CH(alkyl)-, L³ is —CH₂—, R⁶ is substituted or unsubstituted benzyl, Y is substituted or unsubstituted aryl, and X is substituted or unsubstituted aryl.

In another embodiment of the compounds of formula (IC-2), L⁴ is —CH₂—, L³ is —CH(alkyl)-, R⁶ is substituted or unsubstituted benzyl, Y is substituted or unsubstituted aryl, and X is substituted or unsubstituted aryl.

In another embodiment of the compounds of formula (IC-2), L⁴ is —CH(alkyl)-, L³ is —CH(alkyl)-, R⁶ is substituted or unsubstituted benzyl, Y is substituted or unsubstituted aryl, and X is substituted or unsubstituted aryl.

In another embodiment of the compounds of formula (IC-2), L⁴ is —C(R⁴)₂— and L³ is —C(O)—, R⁶ is substituted or unsubstituted benzyl, Y is substituted or unsubstituted aryl, and X is substituted or unsubstituted aryl.

In another embodiment of the compounds of formula (IC-2), L⁴ is —CH₂—, L³ is —C(O)—, R⁶ is substituted or unsubstituted benzyl, and Y is substituted or unsubstituted aryl, and X is substituted or unsubstituted aryl.

In another embodiment of the compounds of formula (IC-2), L⁴ is —CH(alkyl)-, L³ is —C(O)—, R⁶ is substituted or unsubstituted benzyl, Y is substituted or unsubstituted aryl, and X is substituted or unsubstituted aryl.

In another embodiment of the compounds of formula (IC-2), L⁴ is —C(R⁴)₂—, L³ is —C(R⁴)₂—, R⁶ is substituted or unsubstituted heteroarylalkyl, Y is substituted or unsubstituted aryl, and X is substituted or unsubstituted aryl.

In another embodiment of the compounds of formula (IC-2), L⁴ is —CH₂—, L³ is —C(R⁴)₂—, R⁶ is substituted or unsubstituted heteroarylalkyl, Y is substituted or unsubstituted aryl, and X is substituted or unsubstituted aryl.

In another embodiment of the compounds of formula (IC-2), L⁴ is —C(R⁴)₂—, L³ is —CH₂—, R⁶ is substituted or unsubstituted heteroarylalkyl, Y is substituted or unsubstituted aryl, and X is substituted or unsubstituted aryl.

In another embodiment of the compounds of formula (IC-2), L⁴ is —CH₂—, L³ is —CH₂—, R⁶ is substituted or unsubstituted heteroarylalkyl, Y is substituted or unsubstituted aryl, and X is substituted or unsubstituted aryl.

In another embodiment of the compounds of formula (IC-2), L⁴ is —CH(alkyl)-, L³ is —CH₂—, R⁶ is substituted or unsubstituted heteroarylalkyl, Y is substituted or unsubstituted aryl, and X is substituted or unsubstituted aryl.

In another embodiment of the compounds of formula (IC-2), L⁴ is —CH₂—, L³ is —CH(alkyl)-, R⁶ is substituted or unsubstituted heteroarylalkyl, Y is substituted or unsubstituted aryl, and X is substituted or unsubstituted aryl.

In another embodiment of the compounds of formula (IC-2), L⁴ is —CH(alkyl)-, L³ is —CH(alkyl)-, R⁶ is substituted or unsubstituted heteroarylalkyl, Y is substituted or unsubstituted aryl, and X is substituted or unsubstituted aryl.

In another embodiment of the compounds of formula (IC-2), L⁴ is —C(R⁴)₂— and L³ is —C(O)—, R⁶ is substituted or unsubstituted heteroarylalkyl, Y is substituted or unsubstituted aryl, and X is substituted or unsubstituted aryl.

In another embodiment of the compounds of formula (IC-2), L⁴ is —CH₂—, L³ is —C(O)—, R⁶ is substituted or unsubstituted heteroarylalkyl, Y is substituted or unsubstituted aryl, and X is substituted or unsubstituted aryl.

In another embodiment of the compounds of formula (IC-2), L⁴ is —CH(alkyl)-, L³ is —C(O)—, R⁶ is substituted or unsubstituted heteroarylalkyl, Y is substituted or unsubstituted aryl, and X is substituted or unsubstituted aryl.

In another embodiment of the compounds of formula (IC-2), L⁴ is —C(R⁴)₂—, L³ is —C(R⁴)₂—, R⁶ is H, Y is substituted or unsubstituted heteroaryl, and X is substituted or unsubstituted aryl.

In another embodiment of the compounds of formula (IC-2), L⁴ is —CH₂—, L³ is —C(R⁴)₂—, R⁶ is H, Y is substituted or unsubstituted heteroaryl, and X is substituted or unsubstituted aryl.

In another embodiment of the compounds of formula (IC-2), L⁴ is —C(R⁴)₂—, L³ is —CH₂—, R⁶ is H, Y is substituted or unsubstituted heteroaryl, and X is substituted or unsubstituted aryl.

In another embodiment of the compounds of formula (IC-2), L⁴ is —CH₂—, L³ is —CH₂—, R⁶ is H, Y is substituted or unsubstituted heteroaryl, and X is substituted or unsubstituted aryl.

In another embodiment of the compounds of formula (IC-2), L⁴ is —CH(alkyl)-, L³ is —CH₂—, R⁶ is H, Y is substituted or unsubstituted heteroaryl, and X is substituted or unsubstituted aryl.

In another embodiment of the compounds of formula (IC-2), L⁴ is —CH₂—, L³ is —CH(alkyl)-, R⁶ is H, Y is substituted or unsubstituted heteroaryl, and X is substituted or unsubstituted aryl.

In another embodiment of the compounds of formula (IC-2), L⁴ is —CH(alkyl)-, L³ is —CH(alkyl)-, R⁶ is H, Y is substituted or unsubstituted heteroaryl, and X is substituted or unsubstituted aryl.

In another embodiment of the compounds of formula (IC-2), L⁴ is —C(R⁴)₂— and L³ is —C(O)—, R⁶ is H, Y is substituted or unsubstituted heteroaryl, and X is substituted or unsubstituted aryl.

In another embodiment of the compounds of formula (IC-2), L⁴ is —CH₂—, L³ is —C(O)—, R⁶ is H, Y is substituted or unsubstituted heteroaryl, and X is substituted or unsubstituted aryl.

In another embodiment of the compounds of formula (IC-2), L⁴ is —CH(alkyl)-, L³ is —C(O)—, R⁶ is H, Y is substituted or unsubstituted heteroaryl, and X is substituted or unsubstituted aryl.

In another embodiment of the compounds of formula (IC-2), L⁴ is —C(R⁴)₂—, L³ is —C(R⁴)₂—, R⁶ is alkyl, Y is substituted or unsubstituted heteroaryl, and X is substituted or unsubstituted aryl.

In another embodiment of the compounds of formula (IC-2), L⁴ is —CH₂—, L³ is —C(R⁴)₂—, R⁶ is alkyl, Y is substituted or unsubstituted heteroaryl, and X is substituted or unsubstituted aryl.

In another embodiment of the compounds of formula (IC-2), L⁴ is —C(R⁴)₂—, L³ is —CH₂—, R⁶ is alkyl, Y is substituted or unsubstituted heteroaryl, and X is substituted or unsubstituted aryl.

In another embodiment of the compounds of formula (IC-2), L⁴ is —CH₂—, L³ is —CH₂—, R⁶ is alkyl, Y is substituted or unsubstituted heteroaryl, and X is substituted or unsubstituted aryl.

In another embodiment of the compounds of formula (IC-2), L⁴ is —CH(alkyl)-, L³ is —CH₂—, R⁶ is alkyl, Y is substituted or unsubstituted heteroaryl, and X is substituted or unsubstituted aryl.

In another embodiment of the compounds of formula (IC-2), L⁴ is —CH₂—, L³ is —CH(alkyl)-, R⁶ is alkyl, Y is substituted or unsubstituted heteroaryl, and X is substituted or unsubstituted aryl.

In another embodiment of the compounds of formula (IC-2), L⁴ is —CH(alkyl)-, L³ is —CH(alkyl)-, R⁶ is alkyl, Y is substituted or unsubstituted heteroaryl, and X is substituted or unsubstituted aryl.

In another embodiment of the compounds of formula (IC-2), L⁴ is —C(R⁴)₂— and L³ is —C(O)—, R⁶ is alkyl, Y is substituted or unsubstituted heteroaryl, and X is substituted or unsubstituted aryl.

In another embodiment of the compounds of formula (IC-2), L⁴ is —CH₂—, L³ is —C(O)—, R⁶ is alkyl, Y is substituted or unsubstituted heteroaryl, and X is substituted or unsubstituted aryl.

In another embodiment of the compounds of formula (IC-2), L⁴ is —CH(alkyl)-, L³ is —C(O)—, R⁶ is alkyl, Y is substituted or unsubstituted heteroaryl, and X is substituted or unsubstituted aryl.

In another embodiment of the compounds of formula (IC-2), L⁴ is —C(R⁴)₂—, L³ is —C(R⁴)₂—, R⁶ is substituted or unsubstituted cycloalkyl, Y is substituted or unsubstituted heteroaryl, and X is substituted or unsubstituted aryl.

In another embodiment of the compounds of formula (IC-2), L⁴ is —CH₂—, L³ is —C(R⁴)₂—, R⁶ is substituted or unsubstituted cycloalkyl, Y is substituted or unsubstituted heteroaryl, and X is substituted or unsubstituted aryl.

In another embodiment of the compounds of formula (IC-2), L⁴ is —C(R⁴)₂—, L³ is —CH₂—, R⁶ is substituted or unsubstituted cycloalkyl, Y is substituted or unsubstituted heteroaryl, and X is substituted or unsubstituted aryl.

In another embodiment of the compounds of formula (IC-2), L⁴ is —CH₂—, L³ is —CH₂—, R⁶ is substituted or unsubstituted cycloalkyl, Y is substituted or unsubstituted heteroaryl, and X is substituted or unsubstituted aryl.

In another embodiment of the compounds of formula (IC-2), L⁴ is —CH(alkyl)-, L³ is —CH₂—, R⁶ is substituted or unsubstituted cycloalkyl, Y is substituted or unsubstituted heteroaryl, and X is substituted or unsubstituted aryl.

In another embodiment of the compounds of formula (IC-2), L⁴ is —CH₂—, L³ is —CH(alkyl)-, R⁶ is substituted or unsubstituted cycloalkyl, Y is substituted or unsubstituted heteroaryl, and X is substituted or unsubstituted aryl.

In another embodiment of the compounds of formula (IC-2), L⁴ is —CH(alkyl)-, L³ is —CH(alkyl)-, R⁶ is substituted or unsubstituted cycloalkyl, Y is substituted or unsubstituted heteroaryl, and X is substituted or unsubstituted aryl.

In another embodiment of the compounds of formula (IC-2), L⁴ is —C(R⁴)₂— and L³ is —C(O)—, R⁶ is substituted or unsubstituted cycloalkyl, Y is substituted or unsubstituted heteroaryl, and X is substituted or unsubstituted aryl.

In another embodiment of the compounds of formula (IC-2), L⁴ is —CH₂—, L³ is —C(O)—, R⁶ is substituted or unsubstituted cycloalkyl, Y is substituted or unsubstituted heteroaryl, and X is substituted or unsubstituted aryl.

In another embodiment of the compounds of formula (IC-2), L⁴ is —CH(alkyl)-, L³ is —C(O)—, R⁶ is substituted or unsubstituted cycloalkyl, Y is substituted or unsubstituted heteroaryl, and X is substituted or unsubstituted aryl.

In another embodiment of the compounds of formula (IC-2), L⁴ is —C(R⁴)₂—, L³ is —C(R⁴)₂—, R⁶ is substituted or unsubstituted arylalkyl, Y is substituted or unsubstituted heteroaryl, and X is substituted or unsubstituted aryl.

In another embodiment of the compounds of formula (IC-2), L⁴ is —CH₂—, L³ is —C(R⁴)₂—, R⁶ is substituted or unsubstituted arylalkyl, Y is substituted or unsubstituted heteroaryl, and X is substituted or unsubstituted aryl.

In another embodiment of the compounds of formula (IC-2), L⁴ is —C(R⁴)₂—, L³ is —CH₂—, R⁶ is substituted or unsubstituted arylalkyl, Y is substituted or unsubstituted heteroaryl, and X is substituted or unsubstituted aryl.

In another embodiment of the compounds of formula (IC-2), L⁴ is —CH₂—, L³ is —CH₂—, R⁶ is substituted or unsubstituted arylalkyl, Y is substituted or unsubstituted heteroaryl, and X is substituted or unsubstituted aryl.

In another embodiment of the compounds of formula (IC-2), L⁴ is —CH(alkyl)-, L³ is —CH₂—, R⁶ is substituted or unsubstituted arylalkyl, Y is substituted or unsubstituted heteroaryl, and X is substituted or unsubstituted aryl.

In another embodiment of the compounds of formula (IC-2), L⁴ is —CH₂—, L³ is —CH(alkyl)-, R⁶ is substituted or unsubstituted arylalkyl, Y is substituted or unsubstituted heteroaryl, and X is substituted or unsubstituted aryl.

In another embodiment of the compounds of formula (IC-2), L⁴ is —CH(alkyl)-, L³ is —CH(alkyl)-, R⁶ is substituted or unsubstituted arylalkyl, Y is substituted or unsubstituted heteroaryl, and X is substituted or unsubstituted aryl.

In another embodiment of the compounds of formula (IC-2), L⁴ is —C(R⁴)₂— and L³ is —C(O)—, R⁶ is substituted or unsubstituted arylalkyl, Y is substituted or unsubstituted heteroaryl, and X is substituted or unsubstituted aryl.

In another embodiment of the compounds of formula (IC-2), L⁴ is —CH₂—, L³ is —C(O)—, R⁶ is substituted or unsubstituted arylalkyl, Y is substituted or unsubstituted heteroaryl, and X is substituted or unsubstituted aryl.

In another embodiment of the compounds of formula (IC-2), L⁴ is —CH(alkyl)-, L³ is —C(O)—, R⁶ is substituted or unsubstituted arylalkyl, Y is substituted or unsubstituted heteroaryl, and X is substituted or unsubstituted aryl.

In another embodiment of the compounds of formula (IC-2), L⁴ is —C(R⁴)₂—, L³ is —C(R⁴)₂—, R⁶ is substituted or unsubstituted benzyl, Y is substituted or unsubstituted heteroaryl, and X is substituted or unsubstituted aryl.

In another embodiment of the compounds of formula (IC-2), L⁴ is —CH₂—, L³ is —C(R⁴)₂—, R⁶ is substituted or unsubstituted benzyl, Y is substituted or unsubstituted heteroaryl, and X is substituted or unsubstituted aryl.

In another embodiment of the compounds of formula (IC-2), L⁴ is —C(R⁴)₂—, L³ is —CH₂—, R⁶ is substituted or unsubstituted benzyl, Y is substituted or unsubstituted heteroaryl, and X is substituted or unsubstituted aryl.

In another embodiment of the compounds of formula (IC-2), L⁴ is —CH₂—, L³ is —CH₂—, R⁶ is substituted or unsubstituted benzyl, Y is substituted or unsubstituted heteroaryl, and X is substituted or unsubstituted aryl.

In another embodiment of the compounds of formula (IC-2), L⁴ is —CH(alkyl)-, L³ is —CH₂—, R⁶ is substituted or unsubstituted benzyl, Y is substituted or unsubstituted heteroaryl, and X is substituted or unsubstituted aryl.

In another embodiment of the compounds of formula (IC-2), L⁴ is —CH₂—, L³ is —CH(alkyl)-, R⁶ is substituted or unsubstituted benzyl, Y is substituted or unsubstituted heteroaryl, and X is substituted or unsubstituted aryl.

In another embodiment of the compounds of formula (IC-2), L⁴ is —CH(alkyl)-, L³ is —CH(alkyl)-, R⁶ is substituted or unsubstituted benzyl, Y is substituted or unsubstituted heteroaryl, and X is substituted or unsubstituted aryl.

In another embodiment of the compounds of formula (IC-2), L⁴ is —C(R⁴)₂— and L³ is —C(O)—, R⁶ is substituted or unsubstituted benzyl, Y is substituted or unsubstituted heteroaryl, and X is substituted or unsubstituted aryl.

In another embodiment of the compounds of formula (IC-2), L⁴ is —CH₂—, L³ is —C(O)—, R⁶ is substituted or unsubstituted benzyl, Y is substituted or unsubstituted heteroaryl, and X is substituted or unsubstituted aryl.

In another embodiment of the compounds of formula (IC-2), L⁴ is —CH(alkyl)-, L³ is —C(O)—, R⁶ is substituted or unsubstituted benzyl, Y is substituted or unsubstituted heteroaryl, and X is substituted or unsubstituted aryl.

In another embodiment of the compounds of formula (IC-2), L⁴ is —C(R⁴)₂—, L³ is —C(R⁴)₂—, R⁶ is substituted or unsubstituted heteroarylalkyl, Y is substituted or unsubstituted heteroaryl, and X is substituted or unsubstituted aryl.

In another embodiment of the compounds of formula (IC-2), L⁴ is —CH₂—, L³ is —C(R⁴)₂—, R⁶ is substituted or unsubstituted heteroarylalkyl, Y is substituted or unsubstituted heteroaryl, and X is substituted or unsubstituted aryl.

In another embodiment of the compounds of formula (IC-2), L⁴ is —C(R⁴)₂—, L³ is —CH₂—, R⁶ is substituted or unsubstituted heteroarylalkyl, Y is substituted or unsubstituted heteroaryl, and X is substituted or unsubstituted aryl.

In another embodiment of the compounds of formula (IC-2), L⁴ is —CH₂—, L³ is —CH₂—, R⁶ is substituted or unsubstituted heteroarylalkyl, Y is substituted or unsubstituted heteroaryl, and X is substituted or unsubstituted aryl.

In another embodiment of the compounds of formula (IC-2), L⁴ is —CH(alkyl)-, L³ is —CH₂—, R⁶ is substituted or unsubstituted heteroarylalkyl, Y is substituted or unsubstituted heteroaryl, and X is substituted or unsubstituted aryl.

In another embodiment of the compounds of formula (IC-2), L⁴ is —CH₂—, L³ is —CH(alkyl)-, R⁶ is substituted or unsubstituted heteroarylalkyl, and Y is substituted or unsubstituted heteroaryl, and X is substituted or unsubstituted aryl.

In another embodiment of the compounds of formula (IC-2), L⁴ is —CH(alkyl)-, L³ is —CH(alkyl)-, R⁶ is substituted or unsubstituted heteroarylalkyl, Y is substituted or unsubstituted heteroaryl, and X is substituted or unsubstituted aryl.

In another embodiment of the compounds of formula (IC-2), L⁴ is —C(R⁴)₂— and L³ is —C(O)—, R⁶ is substituted or unsubstituted heteroarylalkyl, Y is substituted or unsubstituted heteroaryl, and X is substituted or unsubstituted aryl.

In another embodiment of the compounds of formula (IC-2), L⁴ is —CH₂—, L³ is —C(O)—, R⁶ is substituted or unsubstituted heteroarylalkyl, Y is substituted or unsubstituted heteroaryl, and X is substituted or unsubstituted aryl.

In another embodiment of the compounds of formula (IC-2), L⁴ is —CH(alkyl)-, L³ is —C(O)—, R⁶ is substituted or unsubstituted heteroarylalkyl, Y is substituted or unsubstituted heteroaryl, and X is substituted or unsubstituted aryl.

In another embodiment of the compounds of formula (IC-2), L⁴ is —C(R⁴)₂—, L³ is —C(R⁴)₂—, R⁶ is H, Y is substituted or unsubstituted aryl, and X is substituted or unsubstituted heteroaryl.

In another embodiment of the compounds of formula (IC-2), L⁴ is —CH₂—, L³ is —C(R⁴)₂—, R⁶ is H, Y is substituted or unsubstituted aryl, and X is substituted or unsubstituted heteroaryl.

In another embodiment of the compounds of formula (IC-2), L⁴ is —C(R⁴)₂—, L³ is —CH₂—, R⁶ is H, Y is substituted or unsubstituted aryl, and X is substituted or unsubstituted heteroaryl.

In another embodiment of the compounds of formula (IC-2), L⁴ is —CH₂—, L³ is —CH₂—, R⁶ is H, Y is substituted or unsubstituted aryl, and X is substituted or unsubstituted heteroaryl.

In another embodiment of the compounds of formula (IC-2), L⁴ is —CH(alkyl)-, L³ is —CH₂—, R⁶ is H, Y is substituted or unsubstituted aryl, and X is substituted or unsubstituted heteroaryl.

In another embodiment of the compounds of formula (IC-2), L⁴ is —CH₂—, L³ is —CH(alkyl)-, R⁶ is H, Y is substituted or unsubstituted aryl, and X is substituted or unsubstituted heteroaryl.

In another embodiment of the compounds of formula (IC-2), L⁴ is —CH(alkyl)-, L³ is —CH(alkyl)-, R⁶ is H, Y is substituted or unsubstituted aryl, and X is substituted or unsubstituted heteroaryl.

In another embodiment of the compounds of formula (IC-2), L⁴ is —C(R⁴)₂— and L³ is —C(O)—, R⁶ is H, Y is substituted or unsubstituted aryl, and X is substituted or unsubstituted heteroaryl.

In another embodiment of the compounds of formula (IC-2), L⁴ is —CH₂—, L³ is —C(O)—, R⁶ is H, Y is substituted or unsubstituted aryl, and X is substituted or unsubstituted heteroaryl.

In another embodiment of the compounds of formula (IC-2), L⁴ is —CH(alkyl)-, L³ is —C(O)—, R⁶ is H, Y is substituted or unsubstituted aryl, and X is substituted or unsubstituted heteroaryl.

In another embodiment of the compounds of formula (IC-2), L⁴ is —C(R⁴)₂—, L³ is —C(R⁴)₂—, R⁶ is alkyl, Y is substituted or unsubstituted aryl, and X is substituted or unsubstituted heteroaryl.

In another embodiment of the compounds of formula (IC-2), L⁴ is —CH₂—, L³ is —C(R⁴)₂—, R⁶ is alkyl, Y is substituted or unsubstituted aryl, and X is substituted or unsubstituted heteroaryl.

In another embodiment of the compounds of formula (IC-2), L⁴ is —C(R⁴)₂—, L³ is —CH₂—, R⁶ is alkyl, Y is substituted or unsubstituted aryl, and X is substituted or unsubstituted heteroaryl.

In another embodiment of the compounds of formula (IC-2), L⁴ is —CH₂—, L³ is —CH₂—, R⁶ is alkyl, Y is substituted or unsubstituted aryl, and X is substituted or unsubstituted heteroaryl.

In another embodiment of the compounds of formula (IC-2), L⁴ is —CH(alkyl)-, L³ is —CH₂—, R⁶ is alkyl, Y is substituted or unsubstituted aryl, and X is substituted or unsubstituted heteroaryl.

In another embodiment of the compounds of formula (IC-2), L⁴ is —CH₂—, L³ is —CH(alkyl)-, R⁶ is alkyl, Y is substituted or unsubstituted aryl, and X is substituted or unsubstituted heteroaryl.

In another embodiment of the compounds of formula (IC-2), L⁴ is —CH(alkyl)-, L³ is —CH(alkyl)-, R⁶ is alkyl, Y is substituted or unsubstituted aryl, and X is substituted or unsubstituted heteroaryl.

In another embodiment of the compounds of formula (IC-2), L⁴ is —C(R⁴)₂— and L³ is —C(O)—, R⁶ is alkyl, Y is substituted or unsubstituted aryl, and X is substituted or unsubstituted heteroaryl.

In another embodiment of the compounds of formula (IC-2), L⁴ is —CH₂—, L³ is —C(O)—, R⁶ is alkyl, Y is substituted or unsubstituted aryl, and X is substituted or unsubstituted heteroaryl.

In another embodiment of the compounds of formula (IC-2), L⁴ is —CH(alkyl)-, L³ is —C(O)—, R⁶ is alkyl, Y is substituted or unsubstituted aryl, and X is substituted or unsubstituted heteroaryl.

In another embodiment of the compounds of formula (IC-2), L⁴ is —C(R⁴)₂—, L³ is —C(R⁴)₂—, R⁶ is substituted or unsubstituted cycloalkyl, Y is substituted or unsubstituted aryl, and X is substituted or unsubstituted heteroaryl.

In another embodiment of the compounds of formula (IC-2), L⁴ is —CH₂—, L³ is —C(R⁴)₂—, R⁶ is substituted or unsubstituted cycloalkyl, Y is substituted or unsubstituted aryl, and X is substituted or unsubstituted heteroaryl.

In another embodiment of the compounds of formula (IC-2), L⁴ is —C(R⁴)₂—, L³ is —CH₂—, R⁶ is substituted or unsubstituted cycloalkyl, Y is substituted or unsubstituted aryl, and X is substituted or unsubstituted heteroaryl.

In another embodiment of the compounds of formula (IC-2), L⁴ is —CH₂—, L³ is —CH₂—, R⁶ is substituted or unsubstituted cycloalkyl, Y is substituted or unsubstituted aryl, and X is substituted or unsubstituted heteroaryl.

In another embodiment of the compounds of formula (IC-2), L⁴ is —CH(alkyl)-, L³ is —CH₂—, R⁶ is substituted or unsubstituted cycloalkyl, Y is substituted or unsubstituted aryl, and X is substituted or unsubstituted heteroaryl.

In another embodiment of the compounds of formula (IC-2), L⁴ is —CH₂—, L³ is —CH(alkyl)-, R⁶ is substituted or unsubstituted cycloalkyl, Y is substituted or unsubstituted aryl, and X is substituted or unsubstituted heteroaryl.

In another embodiment of the compounds of formula (IC-2), L⁴ is —CH(alkyl)-, L³ is —CH(alkyl)-, R⁶ is substituted or unsubstituted cycloalkyl, Y is substituted or unsubstituted aryl, and X is substituted or unsubstituted heteroaryl.

In another embodiment of the compounds of formula (IC-2), L⁴ is —C(R⁴)₂— and L³ is —C(O)—, R⁶ is substituted or unsubstituted cycloalkyl, Y is substituted or unsubstituted aryl, and X is substituted or unsubstituted heteroaryl.

In another embodiment of the compounds of formula (IC-2), L⁴ is —CH₂—, L³ is —C(O)—, R⁶ is substituted or unsubstituted cycloalkyl, Y is substituted or unsubstituted aryl, and X is substituted or unsubstituted heteroaryl.

In another embodiment of the compounds of formula (IC-2), L⁴ is —CH(alkyl)-, L³ is —C(O)—, R⁶ is substituted or unsubstituted cycloalkyl, Y is substituted or unsubstituted aryl, and X is substituted or unsubstituted heteroaryl.

In another embodiment of the compounds of formula (IC-2), L⁴ is —C(R⁴)₂—, L³ is —C(R⁴)₂—, R⁶ is substituted or unsubstituted arylalkyl, Y is substituted or unsubstituted aryl, and X is substituted or unsubstituted heteroaryl.

In another embodiment of the compounds of formula (IC-2), L⁴ is —CH₂—, L³ is —C(R⁴)₂—, R⁶ is substituted or unsubstituted arylalkyl, Y is substituted or unsubstituted aryl, and X is substituted or unsubstituted heteroaryl.

In another embodiment of the compounds of formula (IC-2), L⁴ is —C(R⁴)₂—, L³ is —CH₂—, R⁶ is substituted or unsubstituted arylalkyl, Y is substituted or unsubstituted aryl, and X is substituted or unsubstituted heteroaryl.

In another embodiment of the compounds of formula (IC-2), L⁴ is —CH₂—, L³ is —CH₂—, R⁶ is substituted or unsubstituted arylalkyl, Y is substituted or unsubstituted aryl, and X is substituted or unsubstituted heteroaryl.

In another embodiment of the compounds of formula (IC-2), L⁴ is —CH(alkyl)-, L³ is —CH₂—, R⁶ is substituted or unsubstituted arylalkyl, Y is substituted or unsubstituted aryl, and X is substituted or unsubstituted heteroaryl.

In another embodiment of the compounds of formula (IC-2), L⁴ is —CH₂—, L³ is —CH(alkyl)-, R⁶ is substituted or unsubstituted arylalkyl, Y is substituted or unsubstituted aryl, and X is substituted or unsubstituted heteroaryl.

In another embodiment of the compounds of formula (IC-2), L⁴ is —CH(alkyl)-, L³ is —CH(alkyl)-, R⁶ is substituted or unsubstituted arylalkyl, Y is substituted or unsubstituted aryl, and X is substituted or unsubstituted heteroaryl.

In another embodiment of the compounds of formula (IC-2), L⁴ is —C(R⁴)₂— and L³ is —C(O)—, R⁶ is substituted or unsubstituted arylalkyl, Y is substituted or unsubstituted aryl, and X is substituted or unsubstituted heteroaryl.

In another embodiment of the compounds of formula (IC-2), L⁴ is —CH₂—, L³ is —C(O)—, R⁶ is substituted or unsubstituted arylalkyl, Y is substituted or unsubstituted aryl, and X is substituted or unsubstituted heteroaryl.

In another embodiment of the compounds of formula (IC-2), L⁴ is —CH(alkyl)-, L³ is —C(O)—, R⁶ is substituted or unsubstituted arylalkyl, Y is substituted or unsubstituted aryl, and X is substituted or unsubstituted heteroaryl.

In another embodiment of the compounds of formula (IC-2), L⁴ is —C(R⁴)₂—, L³ is —C(R⁴)₂—, R⁶ is substituted or unsubstituted benzyl, Y is substituted or unsubstituted aryl, and X is substituted or unsubstituted heteroaryl.

In another embodiment of the compounds of formula (IC-2), L⁴ is —CH₂—, L³ is —C(R⁴)₂—, R⁶ is substituted or unsubstituted benzyl, and Y is substituted or unsubstituted aryl, and X is substituted or unsubstituted heteroaryl.

In another embodiment of the compounds of formula (IC-2), L⁴ is —C(R⁴)₂—, L³ is —CH₂—, R⁶ is substituted or unsubstituted benzyl, Y is substituted or unsubstituted aryl, and X is substituted or unsubstituted heteroaryl.

In another embodiment of the compounds of formula (IC-2), L⁴ is —CH₂—, L³ is —CH₂—, R⁶ is substituted or unsubstituted benzyl, Y is substituted or unsubstituted aryl, and X is substituted or unsubstituted heteroaryl.

In another embodiment of the compounds of formula (IC-2), L⁴ is —CH(alkyl)-, L³ is —CH₂—, R⁶ is substituted or unsubstituted benzyl, Y is substituted or unsubstituted aryl, and X is substituted or unsubstituted heteroaryl.

In another embodiment of the compounds of formula (IC-2), L⁴ is —CH₂—, L³ is —CH(alkyl)-, R⁶ is substituted or unsubstituted benzyl, Y is substituted or unsubstituted aryl, and X is substituted or unsubstituted heteroaryl.

In another embodiment of the compounds of formula (IC-2), L⁴ is —CH(alkyl)-, L³ is —CH(alkyl)-, R⁶ is substituted or unsubstituted benzyl, Y is substituted or unsubstituted aryl, and X is substituted or unsubstituted heteroaryl.

In another embodiment of the compounds of formula (IC-2), L⁴ is —C(R⁴)₂— and L³ is —C(O)—, R⁶ is substituted or unsubstituted benzyl, Y is substituted or unsubstituted aryl, and X is substituted or unsubstituted heteroaryl.

In another embodiment of the compounds of formula (IC-2), L⁴ is —CH₂—, L³ is —C(O)—, R⁶ is substituted or unsubstituted benzyl, and Y is substituted or unsubstituted aryl, and X is substituted or unsubstituted heteroaryl.

In another embodiment of the compounds of formula (IC-2), L⁴ is —CH(alkyl)-, L³ is —C(O)—, R⁶ is substituted or unsubstituted benzyl, Y is substituted or unsubstituted aryl, and X is substituted or unsubstituted heteroaryl.

In another embodiment of the compounds of formula (IC-2), L⁴ is —C(R⁴)₂—, L³ is —C(R⁴)₂—, R⁶ is substituted or unsubstituted heteroarylalkyl, Y is substituted or unsubstituted aryl, and X is substituted or unsubstituted heteroaryl.

In another embodiment of the compounds of formula (IC-2), L⁴ is —CH₂—, L³ is —C(R⁴)₂—, R⁶ is substituted or unsubstituted heteroarylalkyl, Y is substituted or unsubstituted aryl, and X is substituted or unsubstituted heteroaryl.

In another embodiment of the compounds of formula (IC-2), L⁴ is —C(R⁴)₂—, L³ is —CH₂—, R⁶ is substituted or unsubstituted heteroarylalkyl, Y is substituted or unsubstituted aryl, and X is substituted or unsubstituted heteroaryl.

In another embodiment of the compounds of formula (IC-2), L⁴ is —CH₂—, L³ is —CH₂—, R⁶ is substituted or unsubstituted heteroarylalkyl, Y is substituted or unsubstituted aryl, and X is substituted or unsubstituted heteroaryl.

In another embodiment of the compounds of formula (IC-2), L⁴ is —CH(alkyl)-, L³ is —CH₂—, R⁶ is substituted or unsubstituted heteroarylalkyl, Y is substituted or unsubstituted aryl, and X is substituted or unsubstituted heteroaryl.

In another embodiment of the compounds of formula (IC-2), L⁴ is —CH₂—, L³ is —CH(alkyl)-, R⁶ is substituted or unsubstituted heteroarylalkyl, Y is substituted or unsubstituted aryl, and X is substituted or unsubstituted heteroaryl.

In another embodiment of the compounds of formula (IC-2), L⁴ is —CH(alkyl)-, L³ is —CH(alkyl)-, R⁶ is substituted or unsubstituted heteroarylalkyl, Y is substituted or unsubstituted aryl, and X is substituted or unsubstituted heteroaryl.

In another embodiment of the compounds of formula (IC-2), L⁴ is —C(R⁴)₂— and L³ is —C(O)—, R⁶ is substituted or unsubstituted heteroarylalkyl, Y is substituted or unsubstituted aryl, and X is substituted or unsubstituted heteroaryl.

In another embodiment of the compounds of formula (IC-2), L⁴ is —CH₂—, L³ is —C(O)—, R⁶ is substituted or unsubstituted heteroarylalkyl, Y is substituted or unsubstituted aryl, and X is substituted or unsubstituted heteroaryl.

In another embodiment of the compounds of formula (IC-2), L⁴ is —CH(alkyl)-, L³ is —C(O)—, R⁶ is substituted or unsubstituted heteroarylalkyl, Y is substituted or unsubstituted aryl, and X is substituted or unsubstituted heteroaryl.

In another embodiment of the compounds of formula (IC-2), L⁴ is —C(R⁴)₂—, L³ is —C(R⁴)₂—, R⁶ is H, Y is substituted or unsubstituted heteroaryl, and X is substituted or unsubstituted heteroaryl.

In another embodiment of the compounds of formula (IC-2), L⁴ is —CH₂—, L³ is —C(R⁴)₂—, R⁶ is H, Y is substituted or unsubstituted heteroaryl, and X is substituted or unsubstituted heteroaryl.

In another embodiment of the compounds of formula (IC-2), L⁴ is —C(R⁴)₂—, L³ is —CH₂—, R⁶ is H, Y is substituted or unsubstituted heteroaryl, and X is substituted or unsubstituted heteroaryl.

In another embodiment of the compounds of formula (IC-2), L⁴ is —CH₂—, L³ is —CH₂—, R⁶ is H, Y is substituted or unsubstituted heteroaryl, and X is substituted or unsubstituted heteroaryl.

In another embodiment of the compounds of formula (IC-2), L⁴ is —CH(alkyl)-, L³ is —CH₂—, R⁶ is H, Y is substituted or unsubstituted heteroaryl, and X is substituted or unsubstituted heteroaryl.

In another embodiment of the compounds of formula (IC-2), L⁴ is —CH₂—, L³ is —CH(alkyl)-, R⁶ is H, Y is substituted or unsubstituted heteroaryl, and X is substituted or unsubstituted heteroaryl.

In another embodiment of the compounds of formula (IC-2), L⁴ is —CH(alkyl)-, L³ is —CH(alkyl)-, R⁶ is H, Y is substituted or unsubstituted heteroaryl, and X is substituted or unsubstituted heteroaryl.

In another embodiment of the compounds of formula (IC-2), L⁴ is —C(R⁴)₂— and L³ is —C(O)—, R⁶ is H, Y is substituted or unsubstituted heteroaryl, and X is substituted or unsubstituted heteroaryl.

In another embodiment of the compounds of formula (IC-2), L⁴ is —CH₂—, L³ is —C(O)—, R⁶ is H, Y is substituted or unsubstituted heteroaryl, and X is substituted or unsubstituted heteroaryl.

In another embodiment of the compounds of formula (IC-2), L⁴ is —CH(alkyl)-, L³ is —C(O)—, R⁶ is H, Y is substituted or unsubstituted heteroaryl, and X is substituted or unsubstituted heteroaryl.

In another embodiment of the compounds of formula (IC-2), L⁴ is —C(R⁴)₂—, L³ is —C(R⁴)₂—, R⁶ is alkyl, Y is substituted or unsubstituted heteroaryl, and X is substituted or unsubstituted heteroaryl.

In another embodiment of the compounds of formula (IC-2), L⁴ is —CH₂—, L³ is —C(R⁴)₂—, R⁶ is alkyl, Y is substituted or unsubstituted heteroaryl, and X is substituted or unsubstituted heteroaryl.

In another embodiment of the compounds of formula (IC-2), L⁴ is —C(R⁴)₂—, L³ is —CH₂—, R⁶ is alkyl, Y is substituted or unsubstituted heteroaryl, and X is substituted or unsubstituted heteroaryl.

In another embodiment of the compounds of formula (IC-2), L⁴ is —CH₂—, L³ is —CH₂—, R⁶ is alkyl, Y is substituted or unsubstituted heteroaryl, and X is substituted or unsubstituted heteroaryl.

In another embodiment of the compounds of formula (IC-2), L⁴ is —CH(alkyl)-, L³ is —CH₂—, R⁶ is alkyl, Y is substituted or unsubstituted heteroaryl, and X is substituted or unsubstituted heteroaryl.

In another embodiment of the compounds of formula (IC-2), L⁴ is —CH₂—, L³ is —CH(alkyl)-, R⁶ is alkyl, Y is substituted or unsubstituted heteroaryl, and X is substituted or unsubstituted heteroaryl.

In another embodiment of the compounds of formula (IC-2), L⁴ is —CH(alkyl)-, L³ is —CH(alkyl)-, R⁶ is alkyl, Y is substituted or unsubstituted heteroaryl, and X is substituted or unsubstituted heteroaryl.

In another embodiment of the compounds of formula (IC-2), L⁴ is —C(R⁴)₂— and L³ is —C(O)—, R⁶ is alkyl, Y is substituted or unsubstituted heteroaryl, and X is substituted or unsubstituted heteroaryl.

In another embodiment of the compounds of formula (IC-2), L⁴ is —CH₂—, L³ is —C(O)—, R⁶ is alkyl, Y is substituted or unsubstituted heteroaryl, and X is substituted or unsubstituted heteroaryl.

In another embodiment of the compounds of formula (IC-2), L⁴ is —CH(alkyl)-, L³ is —C(O)—, R⁶ is alkyl, Y is substituted or unsubstituted heteroaryl, and X is substituted or unsubstituted heteroaryl.

In another embodiment of the compounds of formula (IC-2), L⁴ is —C(R⁴)₂—, L³ is —C(R⁴)₂—, R⁶ is substituted or unsubstituted cycloalkyl, Y is substituted or unsubstituted heteroaryl, and X is substituted or unsubstituted heteroaryl.

In another embodiment of the compounds of formula (IC-2), L⁴ is —CH₂—, L³ is —C(R⁴)₂—, R⁶ is substituted or unsubstituted cycloalkyl, Y is substituted or unsubstituted heteroaryl, and X is substituted or unsubstituted heteroaryl.

In another embodiment of the compounds of formula (IC-2), L⁴ is —C(R⁴)₂—, L³ is —CH₂—, R⁶ is substituted or unsubstituted cycloalkyl, Y is substituted or unsubstituted heteroaryl, and X is substituted or unsubstituted heteroaryl.

In another embodiment of the compounds of formula (IC-2), L⁴ is —CH₂—, L³ is —CH₂—, R⁶ is substituted or unsubstituted cycloalkyl, Y is substituted or unsubstituted heteroaryl, and X is substituted or unsubstituted heteroaryl.

In another embodiment of the compounds of formula (IC-2), L⁴ is —CH(alkyl)-, L³ is —CH₂—, R⁶ is substituted or unsubstituted cycloalkyl, Y is substituted or unsubstituted heteroaryl, and X is substituted or unsubstituted heteroaryl.

In another embodiment of the compounds of formula (IC-2), L⁴ is —CH₂—, L³ is —CH(alkyl)-, R⁶ is substituted or unsubstituted cycloalkyl, Y is substituted or unsubstituted heteroaryl, and X is substituted or unsubstituted heteroaryl.

In another embodiment of the compounds of formula (IC-2), L⁴ is —CH(alkyl)-, L³ is —CH(alkyl)-, R⁶ is substituted or unsubstituted cycloalkyl, Y is substituted or unsubstituted heteroaryl, and X is substituted or unsubstituted heteroaryl.

In another embodiment of the compounds of formula (IC-2), L⁴ is —C(R⁴)₂— and L³ is —C(O)—, R⁶ is substituted or unsubstituted cycloalkyl, Y is substituted or unsubstituted heteroaryl, and X is substituted or unsubstituted heteroaryl.

In another embodiment of the compounds of formula (IC-2), L⁴ is —CH₂—, L³ is —C(O)—, R⁶ is substituted or unsubstituted cycloalkyl, Y is substituted or unsubstituted heteroaryl, and X is substituted or unsubstituted heteroaryl.

In another embodiment of the compounds of formula (IC-2), L⁴ is —CH(alkyl)-, L³ is —C(O)—, R⁶ is substituted or unsubstituted cycloalkyl, Y is substituted or unsubstituted heteroaryl, and X is substituted or unsubstituted heteroaryl.

In another embodiment of the compounds of formula (IC-2), L⁴ is —C(R⁴)₂—, L³ is —C(R⁴)₂—, R⁶ is substituted or unsubstituted arylalkyl, Y is substituted or unsubstituted heteroaryl, and X is substituted or unsubstituted heteroaryl.

In another embodiment of the compounds of formula (IC-2), L⁴ is —CH₂—, L³ is —C(R⁴)₂—, R⁶ is substituted or unsubstituted arylalkyl, Y is substituted or unsubstituted heteroaryl, and X is substituted or unsubstituted heteroaryl.

In another embodiment of the compounds of formula (IC-2), L⁴ is —C(R⁴)₂—, L³ is —CH₂—, R⁶ is substituted or unsubstituted arylalkyl, Y is substituted or unsubstituted heteroaryl, and X is substituted or unsubstituted heteroaryl.

In another embodiment of the compounds of formula (IC-2), L⁴ is —CH₂—, L³ is —CH₂—, R⁶ is substituted or unsubstituted arylalkyl, Y is substituted or unsubstituted heteroaryl, and X is substituted or unsubstituted heteroaryl.

In another embodiment of the compounds of formula (IC-2), L⁴ is —CH(alkyl)-, L³ is —CH₂—, R⁶ is substituted or unsubstituted arylalkyl, Y is substituted or unsubstituted heteroaryl, and X is substituted or unsubstituted heteroaryl.

In another embodiment of the compounds of formula (IC-2), L⁴ is —CH₂—, L³ is —CH(alkyl)-, R⁶ is substituted or unsubstituted arylalkyl, Y is substituted or unsubstituted heteroaryl, and X is substituted or unsubstituted heteroaryl.

In another embodiment of the compounds of formula (IC-2), L⁴ is —CH(alkyl)-, L³ is —CH(alkyl)-, R⁶ is substituted or unsubstituted arylalkyl, Y is substituted or unsubstituted heteroaryl, and X is substituted or unsubstituted heteroaryl.

In another embodiment of the compounds of formula (IC-2), L⁴ is —C(R⁴)₂— and L³ is —C(O)—, R⁶ is substituted or unsubstituted arylalkyl, Y is substituted or unsubstituted heteroaryl, and X is substituted or unsubstituted heteroaryl.

In another embodiment of the compounds of formula (IC-2), L⁴ is —CH₂—, L³ is —C(O)—, R⁶ is substituted or unsubstituted arylalkyl, Y is substituted or unsubstituted heteroaryl, and X is substituted or unsubstituted heteroaryl.

In another embodiment of the compounds of formula (IC-2), L⁴ is —CH(alkyl)-, L³ is —C(O)—, R⁶ is substituted or unsubstituted arylalkyl, Y is substituted or unsubstituted heteroaryl, and X is substituted or unsubstituted heteroaryl.

In another embodiment of the compounds of formula (IC-2), L⁴ is —C(R⁴)₂—, L³ is —C(R⁴)₂—, R⁶ is substituted or unsubstituted benzyl, Y is substituted or unsubstituted heteroaryl, and X is substituted or unsubstituted heteroaryl.

In another embodiment of the compounds of formula (IC-2), L⁴ is —CH₂—, L³ is —C(R⁴)₂—, R⁶ is substituted or unsubstituted benzyl, Y is substituted or unsubstituted heteroaryl, and X is substituted or unsubstituted heteroaryl.

In another embodiment of the compounds of formula (IC-2), L⁴ is —C(R⁴)₂—, L³ is —CH₂—, R⁶ is substituted or unsubstituted benzyl, Y is substituted or unsubstituted heteroaryl, and X is substituted or unsubstituted heteroaryl.

In another embodiment of the compounds of formula (IC-2), L⁴ is —CH₂—, L³ is —CH₂—, R⁶ is substituted or unsubstituted benzyl, Y is substituted or unsubstituted heteroaryl, and X is substituted or unsubstituted heteroaryl.

In another embodiment of the compounds of formula (IC-2), L⁴ is —CH(alkyl)-, L³ is —CH₂—, R⁶ is substituted or unsubstituted benzyl, Y is substituted or unsubstituted heteroaryl, and X is substituted or unsubstituted heteroaryl.

In another embodiment of the compounds of formula (IC-2), L⁴ is —CH₂—, L³ is —CH(alkyl)-, R⁶ is substituted or unsubstituted benzyl, Y is substituted or unsubstituted heteroaryl, and X is substituted or unsubstituted heteroaryl.

In another embodiment of the compounds of formula (IC-2), L⁴ is —CH(alkyl)-, L³ is —CH(alkyl)-, R⁶ is substituted or unsubstituted benzyl, Y is substituted or unsubstituted heteroaryl, and X is substituted or unsubstituted heteroaryl.

In another embodiment of the compounds of formula (IC-2), L⁴ is —C(R⁴)₂— and L³ is —C(O)—, R⁶ is substituted or unsubstituted benzyl, Y is substituted or unsubstituted heteroaryl, and X is substituted or unsubstituted heteroaryl.

In another embodiment of the compounds of formula (IC-2), L⁴ is —CH₂—, L³ is —C(O)—, R⁶ is substituted or unsubstituted benzyl, Y is substituted or unsubstituted heteroaryl, and X is substituted or unsubstituted heteroaryl.

In another embodiment of the compounds of formula (IC-2), L⁴ is —CH(alkyl)-, L³ is —C(O)—, R⁶ is substituted or unsubstituted benzyl, Y is substituted or unsubstituted heteroaryl, and X is substituted or unsubstituted heteroaryl.

In another embodiment of the compounds of formula (IC-2), L⁴ is —C(R⁴)₂—, L³ is —C(R⁴)₂—, R⁶ is substituted or unsubstituted heteroarylalkyl, Y is substituted or unsubstituted heteroaryl, and X is substituted or unsubstituted heteroaryl.

In another embodiment of the compounds of formula (IC-2), L⁴ is —CH₂—, L³ is —C(R⁴)₂—, R⁶ is substituted or unsubstituted heteroarylalkyl, Y is substituted or unsubstituted heteroaryl, and X is substituted or unsubstituted heteroaryl.

In another embodiment of the compounds of formula (IC-2), L⁴ is —C(R⁴)₂—, L³ is —CH₂—, R⁶ is substituted or unsubstituted heteroarylalkyl, Y is substituted or unsubstituted heteroaryl, and X is substituted or unsubstituted heteroaryl.

In another embodiment of the compounds of formula (IC-2), L⁴ is —CH₂—, L³ is —CH₂—, R⁶ is substituted or unsubstituted heteroarylalkyl, Y is substituted or unsubstituted heteroaryl, and X is substituted or unsubstituted heteroaryl.

In another embodiment of the compounds of formula (IC-2), L⁴ is —CH(alkyl)-, L³ is —CH₂—, R⁶ is substituted or unsubstituted heteroarylalkyl, Y is substituted or unsubstituted heteroaryl, and X is substituted or unsubstituted heteroaryl.

In another embodiment of the compounds of formula (IC-2), L⁴ is —CH₂—, L³ is —CH(alkyl)-, R⁶ is substituted or unsubstituted heteroarylalkyl, and Y is substituted or unsubstituted heteroaryl, and X is substituted or unsubstituted heteroaryl.

In another embodiment of the compounds of formula (IC-2), L⁴ is —CH(alkyl)-, L³ is —CH(alkyl)-, R⁶ is substituted or unsubstituted heteroarylalkyl, Y is substituted or unsubstituted heteroaryl, and X is substituted or unsubstituted heteroaryl.

In another embodiment of the compounds of formula (IC-2), L⁴ is —C(R⁴)₂— and L³ is —C(O)—, R⁶ is substituted or unsubstituted heteroarylalkyl, Y is substituted or unsubstituted heteroaryl, and X is substituted or unsubstituted heteroaryl.

In another embodiment of the compounds of formula (IC-2), L⁴ is —CH₂—, L³ is —C(O)—, R⁶ is substituted or unsubstituted heteroarylalkyl, Y is substituted or unsubstituted heteroaryl, and X is substituted or unsubstituted heteroaryl.

In another embodiment of the compounds of formula (IC-2), L⁴ is —CH(alkyl)-, L³ is —C(O)—, R⁶ is substituted or unsubstituted heteroarylalkyl, Y is substituted or unsubstituted heteroaryl, and X is substituted or unsubstituted heteroaryl.

In another embodiment, the compounds of the present invention, or pharmaceutically acceptable salts, solvates, prodrugs, tautomers, or esters thereof, have the structure of formula (IC-3):

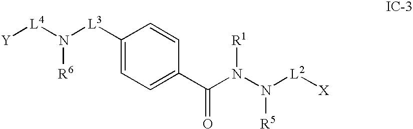

wherein R¹, R⁵, R⁶, L³, L⁴, X and Y are as defined herein.

In another embodiment of the compounds of formula IC-3, L² is a covalent bond, —C(O)—, or —C(O)O—; L³ is —C(R⁴)₂— or —C(O)—; L⁴ is selected from the group consisting of a covalent bond-C(R⁴)₂—, —C(O)—, and —S(O)₂—; X is selected from the group consisting of aminoalkyl, substituted or unsubstituted aryl, substituted or unsubstituted arylalkyl, and substituted or unsubstituted heteroaryl; Y is selected from the group consisting of H, substituted or unsubstituted cycloalkyl, substituted or unsubstituted heteroaryl, substituted or unsubstituted aryl, substituted or unsubstituted heteroarylalkyl, alkyl, aminoalkyl, hydroxyalkyl, and alkoxyalkyl; R¹ is selected from the group consisting of H, alkyl, substituted or unsubstituted arylalkyl, aminoalkyl, alkenyl, and substituted or unsubstituted heteroarylalkyl; each R⁴ is H or alkyl; R⁵ is selected from the group consisting of H, alkyl, aminoalkyl, and alkyl-S(O)₂—; or R⁵ and R¹, together with the nitrogen atoms to which they are each attached, form a substituted or unsubstituted heterocyclyl ring; and R⁶ is selected from the group consisting of H, alkoxyalkyl, hydroxyalkyl, substituted or unsubstituted arylalkyl, and substituted or unsubstituted heteroarylalkyl; or R⁶ and -L⁴-Y, together with the nitrogen atom to which they are both attached, form a substituted or unsubstituted heterocyclyl ring.

In another embodiment of the compounds of formula IC-3, L² is selected from the group consisting of a covalent bond, —C(O)—, and —C(O)O—; L³ is —CH₂— or —C(O)-L⁴ is selected from the group consisting of a covalent bond, —CH₂—, —C(O)—, and —S(O)₂—; X is selected from the group consisting of H, methyl, substituted or unsubstituted phenyl, substituted or unsubstituted pyridyl, and substituted or unsubstituted pyrimidyl; Y is selected from the group consisting of substituted or unsubstituted phenyl, substituted or unsubstituted pyrimidyl, substituted or unsubstituted 2-pyridyl, substituted or unsubstituted imidazolyl, substituted or unsubstituted phthalamidoalkyl, substituted or unsubstituted 5,6,7,8-tetrahydroquinolin-8-yl, aminomethyl, aminoethyl, aminopropyl, aminobutyl, methyl, hydroxyalkyl, and methoxyalkyl; R¹ is selected from the group consisting of H, methyl, substituted or unsubstituted benzyl, aminopropyl, and allyl; R⁵ is selected from the group consisting of H, methyl, aminopropyl, aminobutyl, and methyl-S(O)₂—; or R⁵ and R¹, together with the nitrogen atoms to which they are each attached, form a substituted or unsubstituted 5-membered or 6-membered heterocyclyl ring; R⁶ is selected from the group consisting of H, methoxyalkyl, hydroxyalkyl, substituted or unsubstituted pyridylylalkyl, substituted or unsubstituted cycloalkyl with a fused aromatic ring, substituted or unsubstituted benzyl; or R⁶ and -L⁴-Y, together with the nitrogen atom to which they are both attached, form a substituted or unsubstituted piperazinyl ring.

In another embodiment of the compounds of formula IC-3, L² is a covalent bond.

In another embodiment of the compounds of formula IC-3, X is a substituted or unsubstituted aryl.

In another embodiment of the compounds of formula IC-3, X is a substituted or unsubstituted heteroaryl.

In another embodiment of the compounds of formula IC-3, R⁵ is H.

In another embodiment of the compounds of formula IC-3, R⁵ is alkyl.

In another embodiment of the compounds of formula IC-3, R⁵ is aminoalkyl.

In another embodiment of the compounds of formula IC-3, R⁵ is aminopropyl.

In another embodiment of the compounds of formula IC-3, R⁵ is aminoethyl.

In another embodiment of the compounds of formula IC-3, R⁵ is substituted or unsubstituted arylaminoethyl.

In another embodiment of the compounds of formula IC-3, R⁵ is substituted or unsubstituted arylaminopropyl.

In another embodiment of the compounds of formula IC-3, R⁵ is substituted or unsubstituted heteroarylaminoethyl.

In another embodiment of the compounds of formula IC-3, R⁵ is substituted or unsubstituted heteroarylaminopropyl.

In another embodiment of the compounds of formula IC-3, R⁵ is an amidoalkyl.

In another embodiment of the compounds of formula IC-3, R⁵ is -alkyl-NH—C(O)—NH(alkyl).

In another embodiment of the compounds of formula IC-3, R⁵ is -alkyl-N(alkyl)-C(O)—NH(alkyl).

In another embodiment of the compounds of formula IC-3, R⁵ is alkylaminopropyl.

In another embodiment of the compounds of formula IC-3, R⁵ is dialkylaminopropyl.

In another embodiment of the compounds of formula IC-3, R⁵ is methyl.

In another embodiment of the compounds of formula IC-3, R⁵ is ethyl.

In another embodiment of the compounds of formula IC-3, R⁵ is propyl.

In another embodiment of the compounds of formula IC-3, R⁵ is substituted or unsubstituted benzyl.

In another embodiment of the compounds of formula IC-3, R⁵ is substituted or unsubstituted phenylaminoethyl.

In another embodiment of the compounds of formula IC-3, R⁵ is substituted or unsubstituted phenylaminopropyl.

In another embodiment of the compounds of formula IC-3, R⁵ is substituted or unsubstituted pyridylaminoethyl.

In another embodiment of the compounds of formula IC-3, R⁵ is substituted or unsubstituted pyridylaminopropyl.

In another embodiment of the compounds of formula IC-3, R⁵ is substituted or unsubstituted pyrimidylaminoethyl.

In another embodiment of the compounds of formula IC-3, R⁵ is substituted or unsubstituted pyrimidylaminopropyl.

In another embodiment of the compounds of formula IC-3, L² is a covalent bond and X is a substituted or unsubstituted aryl.

In another embodiment of the compounds of formula IC-3, L² is a covalent bond and X is a substituted or unsubstituted heteroaryl.

In another embodiment of the compounds of formula IC-3, L² is a covalent bond and X is a substituted or unsubstituted phenyl.

In another embodiment of the compounds of formula IC-3, L² is a covalent bond and X is a substituted or unsubstituted pyridyl.

In another embodiment of the compounds of formula IC-3, L² is a covalent bond and X is a substituted or unsubstituted pyrimidyl.

In another embodiment of the compounds of formula IC-3, L² is a covalent bond and X is a substituted or unsubstituted indolyl.

In another embodiment of the compounds of formula IC-3, L² is a covalent bond and X is a substituted or unsubstituted quinolyl.

In another embodiment of the compounds of formula IC-3, L² is a covalent bond and X is a substituted or unsubstituted thienyl.

In another embodiment of the compounds of formula IC-3, L² is a covalent bond and X is a substituted or unsubstituted pyridyl.

In another embodiment of the compounds of formula IC-3, L² is a covalent bond and X is a substituted or unsubstituted purinyl.

In another embodiment of the compounds of formula IC-3, L² is a covalent bond, X is a substituted or unsubstituted aryl, and R⁵ is H.

In another embodiment of the compounds of formula IC-3, L² is a covalent bond, X is a substituted or unsubstituted heteroaryl, and R⁵ is H.

In another embodiment of the compounds of formula IC-3, L² is a covalent bond, X is a substituted or unsubstituted phenyl, and R⁵ is H.

In another embodiment of the compounds of formula IC-3, L² is a covalent bond, X is a substituted or unsubstituted pyridyl, and R⁵ is H.

In another embodiment of the compounds of formula IC-3, L² is a covalent bond, X is a substituted or unsubstituted pyrimidyl, and R⁵ is H.

In another embodiment of the compounds of formula IC-3, L² is a covalent bond, X is a substituted or unsubstituted indolyl, and R⁵ is H.

In another embodiment of the compounds of formula IC-3, L² is a covalent bond, X is a substituted or unsubstituted quinolyl, and R⁵ is H.

In another embodiment of the compounds of formula IC-3, L² is a covalent bond, X is a substituted or unsubstituted thienyl, and R⁵ is H.

In another embodiment of the compounds of formula IC-3, L² is a covalent bond, X is a substituted or unsubstituted pyridyl, and R⁵ is H.

In another embodiment of the compounds of formula IC-3, L² is a covalent bond, X is a substituted or unsubstituted purinyl, and R⁵ is H.

In another embodiment of the compounds of formula IC-3, L² is a covalent bond, X is a substituted or unsubstituted aryl, and R⁵ is alkyl.

In another embodiment of the compounds of formula IC-3, L² is a covalent bond, X is a substituted or unsubstituted heteroaryl, and R⁵ is alkyl.

In another embodiment of the compounds of formula IC-3, L² is a covalent bond, X is a substituted or unsubstituted phenyl, and R⁵ is alkyl.

In another embodiment of the compounds of formula IC-3, L² is a covalent bond, X is a substituted or unsubstituted pyridyl, and R⁵ is alkyl.

In another embodiment of the compounds of formula IC-3, L² is a covalent bond, X is a substituted or unsubstituted pyrimidyl, and R⁵ is alkyl.

In another embodiment of the compounds of formula IC-3, L² is a covalent bond, X is a substituted or unsubstituted indolyl, and R⁵ is alkyl.

In another embodiment of the compounds of formula IC-3, L² is a covalent bond, X is a substituted or unsubstituted quinolyl, and R⁵ is alkyl.

In another embodiment of the compounds of formula IC-3, L² is a covalent bond, X is a substituted or unsubstituted thienyl, and R⁵ is alkyl.

In another embodiment of the compounds of formula IC-3, L² is a covalent bond, X is a substituted or unsubstituted pyridyl, and R⁵ is alkyl.

In another embodiment of the compounds of formula IC-3, L² is a covalent bond, X is a substituted or unsubstituted purinyl, and R⁵ is alkyl.

In another embodiment of the compounds of formula IC-3, L² is a covalent bond, X is a substituted or unsubstituted aryl, and R⁵ is methyl.

In another embodiment of the compounds of formula IC-3, L² is a covalent bond, X is a substituted or unsubstituted heteroaryl, and R⁵ is methyl.

In another embodiment of the compounds of formula IC-3, L² is a covalent bond, X is a substituted or unsubstituted phenyl, and R⁵ is methyl.

In another embodiment of the compounds of formula IC-3, L² is a covalent bond, X is a substituted or unsubstituted pyridyl, and R⁵ is methyl.

In another embodiment of the compounds of formula IC-3, L² is a covalent bond, X is a substituted or unsubstituted pyrimidyl, and R⁵ is methyl.

In another embodiment of the compounds of formula IC-3, L² is a covalent bond, X is a substituted or unsubstituted indolyl, and R⁵ is methyl.

In another embodiment of the compounds of formula IC-3, L² is a covalent bond, X is a substituted or unsubstituted quinolyl, and R⁵ is methyl.

In another embodiment of the compounds of formula IC-3, L² is a covalent bond, X is a substituted or unsubstituted thienyl, and R⁵ is methyl.

In another embodiment of the compounds of formula IC-3, L² is a covalent bond, X is a substituted or unsubstituted pyridyl, and R⁵ is methyl.

In another embodiment of the compounds of formula IC-3, L² is a covalent bond, X is a substituted or unsubstituted purinyl, and R⁵ is methyl.

In another embodiment of the compounds of formula IC-3, L² is a covalent bond, X is a substituted or unsubstituted aryl, and R⁵ is ethyl.

In another embodiment of the compounds of formula IC-3, L² is a covalent bond, X is a substituted or unsubstituted heteroaryl, and R⁵ is ethyl.

In another embodiment of the compounds of formula IC-3, L² is a covalent bond, X is a substituted or unsubstituted phenyl, and R⁵ is ethyl.

In another embodiment of the compounds of formula IC-3, L² is a covalent bond, X is a substituted or unsubstituted pyridyl, and R⁵ is ethyl.

In another embodiment of the compounds of formula IC-3, L² is a covalent bond, X is a substituted or unsubstituted pyrimidyl, and R⁵ is ethyl.

In another embodiment of the compounds of formula IC-3, L² is a covalent bond, X is a substituted or unsubstituted indolyl, and R⁵ is ethyl.

In another embodiment of the compounds of formula IC-3, L² is a covalent bond, X is a substituted or unsubstituted quinolyl, and R⁵ is ethyl.

In another embodiment of the compounds of formula IC-3, L² is a covalent bond, X is a substituted or unsubstituted thienyl, and R⁵ is ethyl.

In another embodiment of the compounds of formula IC-3, L² is a covalent bond, X is a substituted or unsubstituted pyridyl, and R⁵ is ethyl.

In another embodiment of the compounds of formula IC-3, L² is a covalent bond, X is a substituted or unsubstituted purinyl, and R⁵ is ethyl.

In another embodiment of the compounds of formula IC-3, L² is a covalent bond, X is a substituted or unsubstituted aryl, and R⁵ is substituted or unsubstituted benzyl.

In another embodiment of the compounds of formula IC-3, L² is a covalent bond, X is a substituted or unsubstituted heteroaryl, and R⁵ is substituted or unsubstituted benzyl.

In another embodiment of the compounds of formula IC-3, L² is a covalent bond, X is a substituted or unsubstituted phenyl, and R⁵ is substituted or unsubstituted benzyl.

In another embodiment of the compounds of formula IC-3, L₂ is a covalent bond, X is a substituted or unsubstituted pyridyl, and R⁵ is substituted or unsubstituted benzyl.

In another embodiment of the compounds of formula IC-3, L² is a covalent bond, X is a substituted or unsubstituted pyrimidyl, and R⁵ is substituted or unsubstituted benzyl.

In another embodiment of the compounds of formula IC-3, L² is a covalent bond, X is a substituted or unsubstituted indolyl, and R⁵ is substituted or unsubstituted benzyl.

In another embodiment of the compounds of formula IC-3, L² is a covalent bond, X is a substituted or unsubstituted quinolyl, and R⁵ is substituted or unsubstituted benzyl.

In another embodiment of the compounds of formula IC-3, L² is a covalent bond, X is a substituted or unsubstituted thienyl, and R⁵ is substituted or unsubstituted benzyl.

In another embodiment of the compounds of formula IC-3, L² is a covalent bond, X is a substituted or unsubstituted pyridyl, and R⁵ is substituted or unsubstituted benzyl.

In another embodiment of the compounds of formula IC-3, L² is a covalent bond, X is a substituted or unsubstituted purinyl, and R⁵ is substituted or unsubstituted benzyl.

In another embodiment of the compounds of formula IC-3, L² is a covalent bond, X is a substituted or unsubstituted aryl, and R⁵ is substituted or unsubstituted aminoalkyl, wherein said substituted or unsubstituted aminoalkyl is any aminoalkyl defined or described herein.

In another embodiment of the compounds of formula IC-3, L² is a covalent bond, X is a substituted or unsubstituted heteroaryl, and R⁵ is substituted or unsubstituted aminoalkyl, wherein said aminoalkyl is any substituted or unsubstituted aminoalkyl defined or described herein.

In another embodiment of the compounds of formula IC-3, L² is a covalent bond, X is a substituted or unsubstituted phenyl, and R⁵ is substituted or unsubstituted aminoalkyl, wherein said aminoalkyl is any substituted or unsubstituted aminoalkyl defined or described herein.

In another embodiment of the compounds of formula IC-3, L² is a covalent bond, X is a substituted or unsubstituted pyridyl, and R⁵ is substituted or unsubstituted aminoalkyl, wherein said aminoalkyl is any substituted or unsubstituted aminoalkyl defined or described herein.

In another embodiment of the compounds of formula IC-3, L² is a covalent bond, X is a substituted or unsubstituted pyrimidyl, and R⁵ is substituted or unsubstituted aminoalkyl, wherein said substituted or unsubstituted aminoalkyl is any aminoalkyl defined or described herein.

In another embodiment of the compounds of formula IC-3, L² is a covalent bond, X is a substituted or unsubstituted indolyl, and R⁵ is substituted or unsubstituted aminoalkyl, wherein said aminoalkyl is any substituted or unsubstituted aminoalkyl defined or described herein.

In another embodiment of the compounds of formula IC-3, L² is a covalent bond, X is a substituted or unsubstituted quinolyl, and R⁵ is substituted or unsubstituted aminoalkyl, wherein said aminoalkyl is any substituted or unsubstituted aminoalkyl defined or described herein.

In another embodiment of the compounds of formula IC-3, L² is a covalent bond, X is a substituted or unsubstituted thienyl, and R⁵ is substituted or unsubstituted aminoalkyl, wherein said aminoalkyl is any substituted or unsubstituted aminoalkyl defined or described herein.

In another embodiment of the compounds of formula IC-3, L² is a covalent bond, X is a substituted or unsubstituted pyridyl, and R⁵ is substituted or unsubstituted aminoalkyl, wherein said aminoalkyl is any substituted or unsubstituted aminoalkyl defined or described herein.

In another embodiment of the compounds of formula IC-3, L² is a covalent bond, X is a substituted or unsubstituted purinyl, and R⁵ is substituted or unsubstituted aminoalkyl, wherein said aminoalkyl is any substituted or unsubstituted aminoalkyl defined or described herein.

In another embodiment of the compounds of formula IC-3, L³ is —C(R⁴)₂—.

In another embodiment of the compounds of formula IC-3, L³ is —CH₂—.

In another embodiment of the compounds of formula IC-3, L³ is —CH(CH₃)—.

In another embodiment of the compounds of formula IC-3, L³ is —C(O)—.

In another embodiment of the compounds of formula IC-3, L⁴ is —C(R⁴)₂—.

In another embodiment of the compounds of formula IC-3, L⁴ is —CH₂—.

In another embodiment of the compounds of formula IC-3, L⁴ is —CH(CH₃)—.

In another embodiment of the compounds of formula IC-3, L⁴ is a covalent bond.

In another embodiment of the compounds of formula IC-3, L³ is —C(R⁴)₂— and L⁴ is —C(R⁴)₂—, wherein R⁴ is as defined herein.

In another embodiment of the compounds of formula IC-3, L³ is —CH₂— and L⁴ is —C(R⁴)₂—, wherein R⁴ is as defined herein.

In another embodiment of the compounds of formula IC-3, L³ is —CH(CH₃)— and L⁴ is —C(R⁴)₂—, wherein R⁴ is as defined herein.

In another embodiment of the compounds of formula IC-3, L³ is —C(O)— and L⁴ is —C(R⁴)₂—, wherein R⁴ is as defined herein.

In another embodiment of the compounds of formula IC-3, L³ is —C(R⁴)₂— and L⁴ is —CH(CH₃)—, wherein R⁴ is as defined herein.

In another embodiment of the compounds of formula IC-3, L³ is —CH₂— and L⁴ is —CH(CH₃)—.

In another embodiment of the compounds of formula IC-3, L³ is —CH(CH₃)— and L⁴ is —CH(CH₃)—.

In another embodiment of the compounds of formula IC-3, L³ is —C(O)— and L⁴ is —CH(CH₃)—.

In another embodiment of the compounds of formula IC-3, L³ is —C(R⁴)₂—, wherein R⁴ is as defined herein, and L⁴ is a covalent bond.

In another embodiment of the compounds of formula IC-3, L³ is —CH₂— and L⁴ is a covalent bond.

In another embodiment of the compounds of formula IC-3, L³ is —CH(CH₃)— and L⁴ is a covalent bond.

In another embodiment of the compounds of formula IC-3, L³ is —C(O)— and L⁴ is a covalent bond.

In another embodiment of the compounds of formula IC-3, Y is substituted or unsubstituted aryl.

In another embodiment of the compounds of formula IC-3, Y is substituted or unsubstituted phenyl.

In another embodiment of the compounds of formula IC-3, Y is substituted or unsubstituted heteroaryl.

In another embodiment of the compounds of formula IC-3, Y is substituted or unsubstituted pyridyl.

In another embodiment of the compounds of formula IC-3, Y is substituted or unsubstituted pyrimidyl.

In another embodiment of the compounds of formula IC-3, Y is substituted or unsubstituted thienyl.

In another embodiment of the compounds of formula IC-3, Y is substituted or unsubstituted furanyl.

In another embodiment of the compounds of formula IC-3, Y is substituted or unsubstituted indolyl.

In another embodiment of the compounds of formula IC-3, Y is substituted or unsubstituted purinyl.

In another embodiment of the compounds of formula IC-3, Y is substituted or unsubstituted pyrrolyl.

In another embodiment of the compounds of formula IC-3, Y is alkoxy alkyl.

In another embodiment of the compounds of formula IC-3, Y is methoxyalkyl.

In another embodiment of the compounds of formula IC-3, Y is methoxyalkyl.

In another embodiment of the compounds of formula IC-3, R⁶ is H.

In another embodiment of the compounds of formula IC-3, R⁶ is alkyl.

In another embodiment of the compounds of formula IC-3, R⁶ is methyl.

In another embodiment of the compounds of formula IC-3, R⁶ is alkoxyalkyl.

In another embodiment of the compounds of formula IC-3, R⁶ is methoxyalkyl.

In another embodiment of the compounds of formula IC-3, R⁶ is ethoxyalkyl.

In another embodiment of the compounds of formula IC-3, L³ is —C(R⁴)₂—, L⁴ is —C(R⁴)₂—, wherein R⁴ is as defined herein, and Y is a substituted or unsubstituted aryl.

In another embodiment of the compounds of formula IC-3, L³ is —CH₂—, L⁴ is —C(R⁴)₂—, wherein R⁴ is as defined herein, and Y is a substituted or unsubstituted aryl.

In another embodiment of the compounds of formula IC-3, L³ is —CH(CH₃)—, L⁴ is —C(R⁴)₂— wherein R⁴ is as defined herein, and Y is a substituted or unsubstituted aryl.

In another embodiment of the compounds of formula IC-3, L³ is —C(O)—, L⁴ is —C(R⁴)₂— wherein R⁴ is as defined herein, and Y is a substituted or unsubstituted aryl.

In another embodiment of the compounds of formula IC-3, L³ is —C(R⁴)₂—, L⁴ is —CH(CH₃)— wherein R⁴ is as defined herein, and Y is a substituted or unsubstituted aryl.

In another embodiment of the compounds of formula IC-3, L³ is —CH₂—, L⁴ is —CH(CH₃)—, and Y is a substituted or unsubstituted aryl.

In another embodiment of the compounds of formula IC-3, L³ is —CH(CH₃)—, L⁴ is —CH(CH₃)—, and Y is a substituted or unsubstituted aryl.

In another embodiment of the compounds of formula IC-3, L³ is —C(O)—, L⁴ is —CH(CH₃)—, and Y is a substituted or unsubstituted aryl.

In another embodiment of the compounds of formula IC-3, L³ is —C(R⁴)₂— wherein R⁴ is as defined herein, L⁴ is a covalent bond, and Y is a substituted or unsubstituted aryl.

In another embodiment of the compounds of formula IC-3, L³ is —CH₂—, L⁴ is a covalent bond, and Y is a substituted or unsubstituted aryl.

In another embodiment of the compounds of formula IC-3, L³ is —CH(CH₃)—, L⁴ is a covalent bond, and Y is a substituted or unsubstituted aryl.

In another embodiment of the compounds of formula IC-3, L³ is —C(O)—, L⁴ is a covalent bond, and Y is a substituted or unsubstituted aryl.

In another embodiment of the compounds of formula IC-3, L³ is —C(R⁴)₂—, L⁴ is —C(R⁴)₂—, wherein R⁴ is as defined herein, and Y is a substituted or unsubstituted phenyl.

In another embodiment of the compounds of formula IC-3, L³ is —CH₂—, L⁴ is —C(R⁴)₂—, wherein R⁴ is as defined herein, and Y is a substituted or unsubstituted phenyl.

In another embodiment of the compounds of formula IC-3, L³ is —CH(CH₃)—, L⁴ is —C(R⁴)₂— wherein R⁴ is as defined herein, and Y is a substituted or unsubstituted phenyl.

In another embodiment of the compounds of formula IC-3, L³ is —C(O)—, L⁴ is —C(R⁴)₂— wherein R⁴ is as defined herein, and Y is a substituted or unsubstituted phenyl.

In another embodiment of the compounds of formula IC-3, L³ is —C(R⁴)₂—, L⁴ is —CH(CH₃)— wherein R⁴ is as defined herein, and Y is a substituted or unsubstituted phenyl.

In another embodiment of the compounds of formula IC-3, L³ is —CH₂—, L⁴ is —CH(CH₃)—, and Y is a substituted or unsubstituted phenyl.

In another embodiment of the compounds of formula IC-3, L³ is —CH(CH₃)—, L⁴ is —CH(CH₃)—, and Y is a substituted or unsubstituted phenyl.

In another embodiment of the compounds of formula IC-3, L³ is —C(O)—, L⁴ is —CH(CH₃)—, and Y is a substituted or unsubstituted phenyl.

In another embodiment of the compounds of formula IC-3, L³ is —C(R⁴)₂— wherein R⁴ is as defined herein, L⁴ is a covalent bond, and Y is a substituted or unsubstituted phenyl.

In another embodiment of the compounds of formula IC-3, L³ is —CH₂—, L⁴ is a covalent bond, and Y is a substituted or unsubstituted phenyl.

In another embodiment of the compounds of formula IC-3, L³ is —CH(CH₃)—, L⁴ is a covalent bond, and Y is a substituted or unsubstituted phenyl.

In another embodiment of the compounds of formula IC-3, L³ is —C(O)—, L⁴ is a covalent bond, and Y is a substituted or unsubstituted phenyl.

In another embodiment of the compounds of formula IC-3, L³ is —C(R⁴)₂—, L⁴ is —C(R⁴)₂—, wherein R⁴ is as defined herein, and Y is a substituted or unsubstituted heteroaryl.

In another embodiment of the compounds of formula IC-3, L³ is —CH₂—, L⁴ is —C(R⁴)₂—, wherein R⁴ is as defined herein, and Y is a substituted or unsubstituted heteroaryl.

In another embodiment of the compounds of formula IC-3, L³ is —CH(CH₃)—, L⁴ is —C(R⁴)₂— wherein R⁴ is as defined herein, and Y is a substituted or unsubstituted heteroaryl.

In another embodiment of the compounds of formula IC-3, L³ is —C(O)—, L⁴ is —C(R⁴)₂— wherein R⁴ is as defined herein, and Y is a substituted or unsubstituted heteroaryl.

In another embodiment of the compounds of formula IC-3, L³ is —C(R⁴)₂—, L⁴ is —CH(CH₃)— wherein R⁴ is as defined herein, and Y is a substituted or unsubstituted heteroaryl.

In another embodiment of the compounds of formula IC-3, L³ is —CH₂—, L⁴ is —CH(CH₃)—, and Y is a substituted or unsubstituted heteroaryl.

In another embodiment of the compounds of formula IC-3, L³ is —CH(CH₃)—, L⁴ is —CH(CH₃)—, and Y is a substituted or unsubstituted heteroaryl.

In another embodiment of the compounds of formula IC-3, L³ is —C(O)—, L⁴ is —CH(CH₃)—, and Y is a substituted or unsubstituted heteroaryl.

In another embodiment of the compounds of formula IC-3, L³ is —C(R⁴)₂— wherein R⁴ is as defined herein, L⁴ is a covalent bond, and Y is a substituted or unsubstituted heteroaryl.

In another embodiment of the compounds of formula IC-3, L³ is —CH₂—, L⁴ is a covalent bond, and Y is a substituted or unsubstituted heteroaryl.

In another embodiment of the compounds of formula IC-3, L³ is —CH(CH₃)—, L⁴ is a covalent bond, and Y is a substituted or unsubstituted heteroaryl.

In another embodiment of the compounds of formula IC-3, L³ is —C(O)—, L⁴ is a covalent bond, and Y is a substituted or unsubstituted heteroaryl.

In another embodiment of the compounds of formula IC-3, L³ is —CH₂—, L⁴ is —C(R⁴)₂—, wherein R⁴ is as defined herein, and Y is a substituted or unsubstituted pyridyl.

In another embodiment of the compounds of formula IC-3, L³ is —CH(CH₃)—, L⁴ is —C(R⁴)₂— wherein R⁴ is as defined herein, and Y is a substituted or unsubstituted pyridyl.

In another embodiment of the compounds of formula IC-3, L³ is —C(O)—, L⁴ is —C(R⁴)₂— wherein R⁴ is as defined herein, and Y is a substituted or unsubstituted pyridyl.

In another embodiment of the compounds of formula IC-3, L³ is —C(R⁴)₂—, L⁴ is —CH(CH₃)— wherein R⁴ is as defined herein, and Y is a substituted or unsubstituted pyridyl.

In another embodiment of the compounds of formula IC-3, L³ is —CH₂—, L⁴ is —CH(CH₃)—, and Y is a substituted or unsubstituted pyridyl.

In another embodiment of the compounds of formula IC-3, L³ is —CH(CH₃)—, L⁴ is —CH(CH₃)—, and Y is a substituted or unsubstituted pyridyl.

In another embodiment of the compounds of formula IC-3, L³ is —C(O)—, L⁴ is —CH(CH₃)—, and Y is a substituted or unsubstituted pyridyl.

In another embodiment of the compounds of formula IC-3, L³ is —C(R⁴)₂— wherein R⁴ is as defined herein, L⁴ is a covalent bond, and Y is a substituted or unsubstituted pyridyl.

In another embodiment of the compounds of formula IC-3, L³ is —CH₂—, L⁴ is a covalent bond, and Y is a substituted or unsubstituted pyridyl.

In another embodiment of the compounds of formula IC-3, L³ is —CH(CH₃)—, L⁴ is a covalent bond, and Y is a substituted or unsubstituted pyridyl.

In another embodiment of the compounds of formula IC-3, L³ is —C(O)—, L⁴ is a covalent bond, and Y is a substituted or unsubstituted pyridyl.

In another embodiment of the compounds of formula IC-3, L³ is —CH₂—, L⁴ is —C(R⁴)₂—, wherein R⁴ is as defined herein, and Y is a substituted or unsubstituted pyrimidyl.

In another embodiment of the compounds of formula IC-3, L³ is —CH(CH₃)—, L⁴ is —C(R⁴)₂— wherein R⁴ is as defined herein, and Y is a substituted or unsubstituted pyrimidyl.

In another embodiment of the compounds of formula IC-3, L³ is —C(O)—, L⁴ is —C(R⁴)₂— wherein R⁴ is as defined herein, and Y is a substituted or unsubstituted pyrimidyl.

In another embodiment of the compounds of formula IC-3, L³ is —C(R⁴)₂—, L⁴ is —CH(CH₃)— wherein R⁴ is as defined herein, and Y is a substituted or unsubstituted pyrimidyl.

In another embodiment of the compounds of formula IC-3, L³ is —CH₂—, L⁴ is —CH(CH₃)—, and Y is a substituted or unsubstituted pyrimidyl.

In another embodiment of the compounds of formula IC-3, L³ is —CH(CH₃)—, L⁴ is —CH(CH₃)—, and Y is a substituted or unsubstituted pyrimidyl.

In another embodiment of the compounds of formula IC-3, L³ is —C(O)—, L⁴ is —CH(CH₃)—, and Y is a substituted or unsubstituted pyrimidyl.

In another embodiment of the compounds of formula IC-3, L³ is —C(R⁴)₂— wherein R⁴ is as defined herein, L⁴ is a covalent bond, and Y is a substituted or unsubstituted pyrimidyl.

In another embodiment of the compounds of formula IC-3, L³ is —CH₂—, L⁴ is a covalent bond, and Y is a substituted or unsubstituted pyrimidyl.

In another embodiment of the compounds of formula IC-3, L³ is —CH(CH₃)—, L⁴ is a covalent bond, and Y is a substituted or unsubstituted pyrimidyl.

In another embodiment of the compounds of formula IC-3, L³ is —C(O)—, L⁴ is a covalent bond, and Y is a substituted or unsubstituted pyrimidyl.

In another embodiment of the compounds of formula IC-3, L³ is —CH₂—, L⁴ is —C(R⁴)₂—, wherein R⁴ is as defined herein, and Y is a substituted or unsubstituted quinolyl.

In another embodiment of the compounds of formula IC-3, L³ is —CH(CH₃)—, L⁴ is —C(R⁴)₂— wherein R⁴ is as defined herein, and Y is a substituted or unsubstituted quinolyl.

In another embodiment of the compounds of formula IC-3, L³ is —C(O)—, L⁴ is —C(R⁴)₂— wherein R⁴ is as defined herein, and Y is a substituted or unsubstituted quinolyl.

In another embodiment of the compounds of formula IC-3, L³ is —C(R⁴)₂—, L⁴ is —CH(CH₃)— wherein R⁴ is as defined herein, and Y is a substituted or unsubstituted quinolyl.

In another embodiment of the compounds of formula IC-3, L³ is —CH₂—, L⁴ is —CH(CH₃)—, and Y is a substituted or unsubstituted quinolyl.

In another embodiment of the compounds of formula IC-3, L³ is —CH(CH₃)—, L⁴ is —CH(CH₃)—, and Y is a substituted or unsubstituted quinolyl.

In another embodiment of the compounds of formula IC-3, L³ is —C(O)—, L⁴ is —CH(CH₃)—, and Y is a substituted or unsubstituted quinolyl.

In another embodiment of the compounds of formula IC-3, L³ is —C(R⁴)₂— wherein R⁴ is as defined herein, L⁴ is a covalent bond, and Y is a substituted or unsubstituted quinolyl.

In another embodiment of the compounds of formula IC-3, L³ is —CH₂—, L⁴ is a covalent bond, and Y is a substituted or unsubstituted quinolyl.

In another embodiment of the compounds of formula IC-3, L³ is —CH(CH₃)—, L⁴ is a covalent bond, and Y is a substituted or unsubstituted quinolyl.

In another embodiment of the compounds of formula IC-3, L³ is —C(O)—, L⁴ is a covalent bond, and Y is a substituted or unsubstituted quinolyl.

In another embodiment of the compounds of formula IC-3, L³ is —CH₂—, L⁴ is —C(R⁴)₂—, wherein R⁴ is as defined herein, and Y is a substituted or unsubstituted indolyl.

In another embodiment of the compounds of formula IC-3, L³ is —CH(CH₃)—, L⁴ is —C(R⁴)₂— wherein R⁴ is as defined herein, and Y is a substituted or unsubstituted indolyl.

In another embodiment of the compounds of formula IC-3, L³ is —C(O)—, L⁴ is —C(R⁴)₂— wherein R⁴ is as defined herein, and Y is a substituted or unsubstituted indolyl.

In another embodiment of the compounds of formula IC-3, L³ is —C(R⁴)₂—, L⁴ is —CH(CH₃)— wherein R⁴ is as defined herein, and Y is a substituted or unsubstituted indolyl.

In another embodiment of the compounds of formula IC-3, L³ is —CH₂—, L⁴ is —CH(CH₃)—, and Y is a substituted or unsubstituted indolyl.

In another embodiment of the compounds of formula IC-3, L³ is —CH(CH₃)—, L⁴ is —CH(CH₃)—, and Y is a substituted or unsubstituted indolyl.

In another embodiment of the compounds of formula IC-3, L³ is —C(O)—, L⁴ is —CH(CH₃)—, and Y is a substituted or unsubstituted indolyl.

In another embodiment of the compounds of formula IC-3, L³ is —C(R⁴)₂— wherein R⁴ is as defined herein, L⁴ is a covalent bond, and Y is a substituted or unsubstituted indolyl.

In another embodiment of the compounds of formula IC-3, L³ is —CH₂—, L⁴ is a covalent bond, and Y is a substituted or unsubstituted indolyl.

In another embodiment of the compounds of formula IC-3, L³ is —CH(CH₃)—, L⁴ is a covalent bond, and Y is a substituted or unsubstituted indolyl.

In another embodiment of the compounds of formula IC-3, L³ is —C(O)—, L⁴ is a covalent bond, and Y is a substituted or unsubstituted indolyl.

In another embodiment of the compounds of formula IC-3, L³ is —CH₂—, L⁴ is —C(R⁴)₂—, wherein R⁴ is as defined herein, and Y is a substituted or unsubstituted thienyl.

In another embodiment of the compounds of formula IC-3, L³ is —CH(CH₃)—, L⁴ is —C(R⁴)₂— wherein R⁴ is as defined herein, and Y is a substituted or unsubstituted thienyl.

In another embodiment of the compounds of formula IC-3, L³ is —C(O)—, L⁴ is —C(R⁴)₂— wherein R⁴ is as defined herein, and Y is a substituted or unsubstituted thienyl.

In another embodiment of the compounds of formula IC-3, L³ is —C(R⁴)₂—, L⁴ is —CH(CH₃)— wherein R⁴ is as defined herein, and Y is a substituted or unsubstituted thienyl.

In another embodiment of the compounds of formula IC-3, L³ is —CH₂—, L⁴ is —CH(CH₃)—, and Y is a substituted or unsubstituted thienyl.

In another embodiment of the compounds of formula IC-3, L³ is —CH(CH₃)—, L⁴ is —CH(CH₃)—, and Y is a substituted or unsubstituted thienyl.

In another embodiment of the compounds of formula IC-3, L³ is —C(O)—, L⁴ is —CH(CH₃)—, and Y is a substituted or unsubstituted thienyl.

In another embodiment of the compounds of formula IC-3, L³ is —C(R⁴)₂— wherein R⁴ is as defined herein, L⁴ is a covalent bond, and Y is a substituted or unsubstituted thienyl.

In another embodiment of the compounds of formula IC-3, L³ is —CH₂—, L⁴ is a covalent bond, and Y is a substituted or unsubstituted thienyl.

In another embodiment of the compounds of formula IC-3, L³ is —CH(CH₃)—, L⁴ is a covalent bond, and Y is a substituted or unsubstituted thienyl.

In another embodiment of the compounds of formula IC-3, L³ is —C(O)—, L⁴ is a covalent bond, and Y is a substituted or unsubstituted thienyl.

In another embodiment of the compounds of formula IC-3, L³ is —CH₂—, L⁴ is —C(R⁴)₂—, wherein R⁴ is as defined herein, and Y is a substituted or unsubstituted purinyl.

In another embodiment of the compounds of formula IC-3, L³ is —CH(CH₃)—, L⁴ is —C(R⁴)₂— wherein R⁴ is as defined herein, and Y is a substituted or unsubstituted purinyl.

In another embodiment of the compounds of formula IC-3, L³ is —C(O)—, L⁴ is —C(R⁴)₂— wherein R⁴ is as defined herein, and Y is a substituted or unsubstituted purinyl.

In another embodiment of the compounds of formula IC-3, L³ is —C(R⁴)₂—, L⁴ is —CH(CH₃)— wherein R⁴ is as defined herein, and Y is a substituted or unsubstituted purinyl.

In another embodiment of the compounds of formula IC-3, L³ is —CH₂—, L⁴ is —CH(CH₃)—, and Y is a substituted or unsubstituted purinyl.

In another embodiment of the compounds of formula IC-3, L³ is —CH(CH₃)—, L⁴ is —CH(CH₃)—, and Y is a substituted or unsubstituted purinyl.

In another embodiment of the compounds of formula IC-3, L³ is —C(O)—, L⁴ is —CH(CH₃)—, and Y is a substituted or unsubstituted purinyl.

In another embodiment of the compounds of formula IC-3, L³ is —C(R⁴)₂— wherein R⁴ is as defined herein, L⁴ is a covalent bond, and Y is a substituted or unsubstituted purinyl.

In another embodiment of the compounds of formula IC-3, L³ is —CH₂—, L⁴ is a covalent bond, and Y is a substituted or unsubstituted purinyl.

In another embodiment of the compounds of formula IC-3, L³ is —CH(CH₃)—, L⁴ is a covalent bond, and Y is a substituted or unsubstituted purinyl.

In another embodiment of the compounds of formula IC-3, L³ is —C(O)—, L⁴ is a covalent bond, and Y is a substituted or unsubstituted purinyl.

In another embodiment of the compounds of formula IC-3, L³ is —CH₂—, L⁴ is —C(R⁴)₂—, wherein R⁴ is as defined herein, and Y is a substituted or unsubstituted furanyl.

In another embodiment of the compounds of formula IC-3, L³ is —CH(CH₃)—, L⁴ is —C(R⁴)₂— wherein R⁴ is as defined herein, and Y is a substituted or unsubstituted furanyl.

In another embodiment of the compounds of formula IC-3, L³ is —C(O)—, L⁴ is —C(R⁴)₂— wherein R⁴ is as defined herein, and Y is a substituted or unsubstituted furanyl.

In another embodiment of the compounds of formula IC-3, L³ is —C(R⁴)₂—, L⁴ is —CH(CH₃)— wherein R⁴ is as defined herein, and Y is a substituted or unsubstituted furanyl.

In another embodiment of the compounds of formula IC-3, L³ is —CH₂—, L⁴ is —CH(CH₃)—, and Y is a substituted or unsubstituted furanyl.

In another embodiment of the compounds of formula IC-3, L³ is —CH(CH₃)—, L⁴ is —CH(CH₃)—, and Y is a substituted or unsubstituted furanyl.

In another embodiment of the compounds of formula IC-3, L³ is —C(O)—, L⁴ is —CH(CH₃)—, and Y is a substituted or unsubstituted furanyl.

In another embodiment of the compounds of formula IC-3, L³ is —C(R⁴)₂— wherein R⁴ is as defined herein, L⁴ is a covalent bond, and Y is a substituted or unsubstituted furanyl.

In another embodiment of the compounds of formula IC-3, L³ is —CH₂—, L⁴ is a covalent bond, and Y is a substituted or unsubstituted furanyl.

In another embodiment of the compounds of formula IC-3, L³ is —CH(CH₃)—, L⁴ is a covalent bond, and Y is a substituted or unsubstituted furanyl.

In another embodiment of the compounds of formula IC-3, L³ is —C(O)—, L⁴ is a covalent bond, and Y is a substituted or unsubstituted furanyl.

In another embodiment of the compounds of formula IC-3, L³ is —CH₂—, L⁴ is —C(R⁴)₂—, wherein R⁴ is as defined herein, and Y is a substituted or unsubstituted pyrrolyl.

In another embodiment of the compounds of formula IC-3, L³ is —CH(CH₃)—, L⁴ is —C(R⁴)₂— wherein R⁴ is as defined herein, and Y is a substituted or unsubstituted pyrrolyl.

In another embodiment of the compounds of formula IC-3, L³ is —C(O)—, L⁴ is —C(R⁴)₂— wherein R⁴ is as defined herein, and Y is a substituted or unsubstituted pyrrolyl.

In another embodiment of the compounds of formula IC-3, L³ is —C(R⁴)₂—, L⁴ is —CH(CH₃)— wherein R⁴ is as defined herein, and Y is a substituted or unsubstituted pyrrolyl.

In another embodiment of the compounds of formula IC-3, L³ is —CH₂—, L⁴ is —CH(CH₃)—, and Y is a substituted or unsubstituted pyrrolyl.

In another embodiment of the compounds of formula IC-3, L³ is —CH(CH₃)—, L⁴ is —CH(CH₃)—, and Y is a substituted or unsubstituted pyrrolyl.

In another embodiment of the compounds of formula IC-3, L³ is —C(O)—, L⁴ is —CH(CH₃)—, and Y is a substituted or unsubstituted pyrrolyl.

In another embodiment of the compounds of formula IC-3, L³ is —C(R⁴)₂— wherein R⁴ is as defined herein, L⁴ is a covalent bond, and Y is a substituted or unsubstituted pyrrolyl.

In another embodiment of the compounds of formula IC-3, L³ is —CH₂—, L⁴ is a covalent bond, and Y is a substituted or unsubstituted pyrrolyl.

In another embodiment of the compounds of formula IC-3, L³ is —CH(CH₃)—, L⁴ is a covalent bond, and Y is a substituted or unsubstituted pyrrolyl.

In another embodiment of the compounds of formula IC-3, L³ is —C(O)—, L⁴ is a covalent bond, and Y is a substituted or unsubstituted pyrrolyl.

In another embodiment of the compounds of formula IC-3, L³ is —CH₂—, L⁴ is —C(R⁴)₂—, wherein R⁴ is as defined herein, and Y is alkoxyalkyl.

In another embodiment of the compounds of formula IC-3, L³ is —CH(CH₃)—, L⁴ is —C(R⁴)₂— wherein R⁴ is as defined herein, and Y is alkoxyalkyl.

In another embodiment of the compounds of formula IC-3, L³ is —C(O)—, L⁴ is —C(R⁴)₂— wherein R⁴ is as defined herein, and Y is alkoxyalkyl.

In another embodiment of the compounds of formula IC-3, L³ is —C(R⁴)₂—, L⁴ is —CH(CH₃)— wherein R⁴ is as defined herein, and Y is alkoxyalkyl.

In another embodiment of the compounds of formula IC-3, L³ is —CH₂—, L⁴ is —CH(CH₃)—, and Y is alkoxyalkyl.

In another embodiment of the compounds of formula IC-3, L³ is —CH(CH₃)—, L⁴ is —CH(CH₃)—, and Y is alkoxyalkyl.

In another embodiment of the compounds of formula IC-3, L³ is —C(O)—, L⁴ is —CH(CH₃)—, and Y is alkoxyalkyl.

In another embodiment of the compounds of formula IC-3, L³ is —C(R⁴)₂— wherein R⁴ is as defined herein, L⁴ is a covalent bond, and Y is alkoxyalkyl.

In another embodiment of the compounds of formula IC-3, L³ is —CH₂—, L⁴ is a covalent bond, and Y is alkoxyalkyl.

In another embodiment of the compounds of formula IC-3, L³ is —CH(CH₃)—, L⁴ is a covalent bond, and Y is alkoxyalkyl.

In another embodiment of the compounds of formula IC-3, L³ is —C(O)—, L⁴ is a covalent bond, and Y is alkoxyalkyl.

In another embodiment of the compounds of formula IC-3, L³ is —C(R⁴)₂—, L⁴ is —C(R⁴)₂—, wherein R⁴ is as defined herein, R⁶ is H, and Y is a substituted or unsubstituted aryl.

In another embodiment of the compounds of formula IC-3, L³ is —CH₂—, L⁴ is —C(R⁴)₂—, wherein R⁴ is as defined herein, R⁶ is H, and Y is a substituted or unsubstituted aryl.

In another embodiment of the compounds of formula IC-3, L³ is —CH(CH₃)—, L⁴ is —C(R⁴)₂— wherein R⁴ is as defined herein, R⁶ is H, and Y is a substituted or unsubstituted aryl.

In another embodiment of the compounds of formula IC-3, L³ is —C(O)—, L⁴ is —C(R⁴)₂— wherein R⁴ is as defined herein, R⁶ is H, and Y is a substituted or unsubstituted aryl.

In another embodiment of the compounds of formula IC-3, L³ is —C(R⁴)₂—, L⁴ is —CH(CH₃)— wherein R⁴ is as defined herein, R⁶ is H, and Y is a substituted or unsubstituted aryl.

In another embodiment of the compounds of formula IC-3, L³ is —CH₂—, L⁴ is —CH(CH₃)—, R⁶ is H, and Y is a substituted or unsubstituted aryl.

In another embodiment of the compounds of formula IC-3, L³ is —CH(CH₃)—, L⁴ is —CH(CH₃)—, R⁶ is H, and Y is a substituted or unsubstituted aryl.

In another embodiment of the compounds of formula IC-3, L³ is —C(O)—, L⁴ is —CH(CH₃)—, R⁶ is H, and Y is a substituted or unsubstituted aryl.

In another embodiment of the compounds of formula IC-3, L³ is —C(R⁴)₂— wherein R⁴ is as defined herein, L⁴ is a covalent bond, R⁶ is H, and Y is a substituted or unsubstituted aryl.

In another embodiment of the compounds of formula IC-3, L³ is —CH₂—, L⁴ is a covalent bond, R⁶ is H, and Y is a substituted or unsubstituted aryl.

In another embodiment of the compounds of formula IC-3, L³ is —CH(CH₃)—, L⁴ is a covalent bond, R⁶ is H, and Y is a substituted or unsubstituted aryl.

In another embodiment of the compounds of formula IC-3, L³ is —C(O)—, L⁴ is a covalent bond, R⁶ is H, and Y is a substituted or unsubstituted aryl.

In another embodiment of the compounds of formula IC-3, L³ is —C(R⁴)₂—, L⁴ is —C(R⁴)₂—, wherein R⁴ is as defined herein, R⁶ is H, and Y is a substituted or unsubstituted phenyl.

In another embodiment of the compounds of formula IC-3, L³ is —CH₂—, L⁴ is —C(R⁴)₂—, wherein R⁴ is as defined herein, R⁶ is H, and Y is a substituted or unsubstituted phenyl.

In another embodiment of the compounds of formula IC-3, L³ is —CH(CH₃)—, L⁴ is —C(R⁴)₂— wherein R⁴ is as defined herein, R⁶ is H, and Y is a substituted or unsubstituted phenyl.

In another embodiment of the compounds of formula IC-3, L³ is —C(O)—, L⁴ is —C(R⁴)₂— wherein R⁴ is as defined herein, R⁶ is H, and Y is a substituted or unsubstituted phenyl.

In another embodiment of the compounds of formula IC-3, L³ is —C(R⁴)₂—, L⁴ is —CH(CH₃)— wherein R⁴ is as defined herein, R⁶ is H, and Y is a substituted or unsubstituted phenyl.

In another embodiment of the compounds of formula IC-3, L³ is —CH₂—, L⁴ is —CH(CH₃)—, R⁶ is H, and Y is a substituted or unsubstituted phenyl.

In another embodiment of the compounds of formula IC-3, L³ is —CH(CH₃)—, L⁴ is —CH(CH₃)—, R⁶ is H, and Y is a substituted or unsubstituted phenyl.

In another embodiment of the compounds of formula IC-3, L³ is —C(O)—, L⁴ is —CH(CH₃)—, R⁶ is H, and Y is a substituted or unsubstituted phenyl.

In another embodiment of the compounds of formula IC-3, L³ is —C(R⁴)₂— wherein R⁴ is as defined herein, L⁴ is a covalent bond, R⁶ is H, and Y is a substituted or unsubstituted phenyl.

In another embodiment of the compounds of formula IC-3, L³ is —CH₂—, L⁴ is a covalent bond, R⁶ is H, and Y is a substituted or unsubstituted phenyl.

In another embodiment of the compounds of formula IC-3, L³ is —CH(CH₃)—, L⁴ is a covalent bond, R⁶ is H, and Y is a substituted or unsubstituted phenyl.

In another embodiment of the compounds of formula IC-3, L³ is —C(O)—, L⁴ is a covalent bond, R⁶ is H, and Y is a substituted or unsubstituted phenyl.

In another embodiment of the compounds of formula IC-3, L³ is —C(R⁴)₂—, L⁴ is —C(R⁴)₂—, wherein R⁴ is as defined herein, R⁶ is H, and Y is a substituted or unsubstituted heteroaryl.

In another embodiment of the compounds of formula IC-3, L³ is —CH₂—, L⁴ is —C(R⁴)₂—, wherein R⁴ is as defined herein, R⁶ is H, and Y is a substituted or unsubstituted heteroaryl.

In another embodiment of the compounds of formula IC-3, L³ is —CH(CH₃)—, L⁴ is —C(R⁴)₂— wherein R⁴ is as defined herein, R⁶ is H, and Y is a substituted or unsubstituted heteroaryl.

In another embodiment of the compounds of formula IC-3, L³ is —C(O)—, L⁴ is —C(R⁴)₂— wherein R⁴ is as defined herein, R⁶ is H, and Y is a substituted or unsubstituted heteroaryl.

In another embodiment of the compounds of formula IC-3, L³ is —C(R⁴)₂—, L⁴ is —CH(CH₃)— wherein R⁴ is as defined herein, R⁶ is H, and Y is a substituted or unsubstituted heteroaryl.

In another embodiment of the compounds of formula IC-3, L³ is —CH₂—, L⁴ is —CH(CH₃)—, R⁶ is H, and Y is a substituted or unsubstituted heteroaryl.

In another embodiment of the compounds of formula IC-3, L³ is —CH(CH₃)—, L⁴ is —CH(CH₃)—, R⁶ is H, and Y is a substituted or unsubstituted heteroaryl.

In another embodiment of the compounds of formula IC-3, L³ is —C(O)—, L⁴ is —CH(CH₃)—, R⁶ is H, and Y is a substituted or unsubstituted heteroaryl.

In another embodiment of the compounds of formula IC-3, L³ is —C(R⁴)₂— wherein R⁴ is as defined herein, L⁴ is a covalent bond, R⁶ is H, and Y is a substituted or unsubstituted heteroaryl.

In another embodiment of the compounds of formula IC-3, L³ is —CH₂—, L⁴ is a covalent bond, R⁶ is H, and Y is a substituted or unsubstituted heteroaryl.

In another embodiment of the compounds of formula IC-3, L³ is —CH(CH₃)—, L⁴ is a covalent bond, R⁶ is H, and Y is a substituted or unsubstituted heteroaryl.

In another embodiment of the compounds of formula IC-3, L³ is —C(O)—, L⁴ is a covalent bond, R⁶ is H, and Y is a substituted or unsubstituted heteroaryl.

In another embodiment of the compounds of formula IC-3, L³ is —CH₂—, L⁴ is —C(R⁴)₂—, wherein R⁴ is as defined herein, R⁶ is H, and Y is a substituted or unsubstituted pyridyl.

In another embodiment of the compounds of formula IC-3, L³ is —CH(CH₃)—, L⁴ is —C(R⁴)₂— wherein R⁴ is as defined herein, R⁶ is H, and Y is a substituted or unsubstituted pyridyl.

In another embodiment of the compounds of formula IC-3, L³ is —C(O)—, L⁴ is —C(R⁴)₂— wherein R⁴ is as defined herein, R⁶ is H, and Y is a substituted or unsubstituted pyridyl.

In another embodiment of the compounds of formula IC-3, L³ is —C(R⁴)₂—, L⁴ is —CH(CH₃)— wherein R⁴ is as defined herein, R⁶ is H, and Y is a substituted or unsubstituted pyridyl.

In another embodiment of the compounds of formula IC-3, L³ is —CH₂—, L⁴ is —CH(CH₃)—, R⁶ is H, and Y is a substituted or unsubstituted pyridyl.

In another embodiment of the compounds of formula IC-3, L³ is —CH(CH₃)—, L⁴ is —CH(CH₃)—, R⁶ is H, and Y is a substituted or unsubstituted pyridyl.

In another embodiment of the compounds of formula IC-3, L³ is —C(O)—, L⁴ is —CH(CH₃)—, R⁶ is H, and Y is a substituted or unsubstituted pyridyl.

In another embodiment of the compounds of formula IC-3, L³ is —C(R⁴)₂— wherein R⁴ is as defined herein, L⁴ is a covalent bond, R⁶ is H, and Y is a substituted or unsubstituted pyridyl.

In another embodiment of the compounds of formula IC-3, L³ is —CH₂—, L⁴ is a covalent bond, R⁶ is H, and Y is a substituted or unsubstituted pyridyl.

In another embodiment of the compounds of formula IC-3, L³ is —CH(CH₃)—, L⁴ is a covalent bond, R⁶ is H, and Y is a substituted or unsubstituted pyridyl.

In another embodiment of the compounds of formula IC-3, L³ is —C(O)—, L⁴ is a covalent bond, R⁶ is H, and Y is a substituted or unsubstituted pyridyl.

In another embodiment of the compounds of formula IC-3, L³ is —CH₂—, L⁴ is —C(R⁴)₂—, wherein R⁴ is as defined herein, R⁶ is H, and Y is a substituted or unsubstituted pyrimidyl.

In another embodiment of the compounds of formula IC-3, L³ is —CH(CH₃)—, L⁴ is —C(R⁴)₂— wherein R⁴ is as defined herein, R⁶ is H, and Y is a substituted or unsubstituted pyrimidyl.

In another embodiment of the compounds of formula IC-3, L³ is —C(O)—, L⁴ is —C(R⁴)₂— wherein R⁴ is as defined herein, R⁶ is H, and Y is a substituted or unsubstituted pyrimidyl.

In another embodiment of the compounds of formula IC-3, L³ is —C(R⁴)₂—, L⁴ is —CH(CH₃)— wherein R⁴ is as defined herein, R⁶ is H, and Y is a substituted or unsubstituted pyrimidyl.

In another embodiment of the compounds of formula IC-3, L³ is —CH₂—, L⁴ is —CH(CH₃)—, R⁶ is H, and Y is a substituted or unsubstituted pyrimidyl.

In another embodiment of the compounds of formula IC-3, L³ is —CH(CH₃)—, L⁴ is —CH(CH₃)—, R⁶ is H, and Y is a substituted or unsubstituted pyrimidyl.

In another embodiment of the compounds of formula IC-3, L³ is —C(O)—, L⁴ is —CH(CH₃)—, R⁶ is H, and Y is a substituted or unsubstituted pyrimidyl.

In another embodiment of the compounds of formula IC-3, L³ is —C(R⁴)₂— wherein R⁴ is as defined herein, L⁴ is a covalent bond, R⁶ is H, and Y is a substituted or unsubstituted pyrimidyl.

In another embodiment of the compounds of formula IC-3, L³ is —CH₂—, L⁴ is a covalent bond, R⁶ is H, and Y is a substituted or unsubstituted pyrimidyl.

In another embodiment of the compounds of formula IC-3, L³ is —CH(CH₃)—, L⁴ is a covalent bond, R⁶ is H, and Y is a substituted or unsubstituted pyrimidyl.

In another embodiment of the compounds of formula IC-3, L³ is —C(O)—, L⁴ is a covalent bond, R⁶ is H, and Y is a substituted or unsubstituted pyrimidyl.

In another embodiment of the compounds of formula IC-3, L³ is —CH₂—, L⁴ is —C(R⁴)₂—, wherein R⁴ is as defined herein, R⁶ is H, and Y is a substituted or unsubstituted quinolyl.

In another embodiment of the compounds of formula IC-3, L³ is —CH(CH₃)—, L⁴ is —C(R⁴)₂— wherein R⁴ is as defined herein, R⁶ is H, and Y is a substituted or unsubstituted quinolyl.

In another embodiment of the compounds of formula IC-3, L³ is —C(O)—, L⁴ is —C(R⁴)₂— wherein R⁴ is as defined herein, R⁶ is H, and Y is a substituted or unsubstituted quinolyl.

In another embodiment of the compounds of formula IC-3, L³ is —C(R⁴)₂—, L⁴ is —CH(CH₃)— wherein R⁴ is as defined herein, R⁶ is H, and Y is a substituted or unsubstituted quinolyl.

In another embodiment of the compounds of formula IC-3, L³ is —CH₂—, L⁴ is —CH(CH₃)—, R⁶ is H, and Y is a substituted or unsubstituted quinolyl.

In another embodiment of the compounds of formula IC-3, L³ is —CH(CH₃)—, L⁴ is —CH(CH₃)—, R⁶ is H, and Y is a substituted or unsubstituted quinolyl.

In another embodiment of the compounds of formula IC-3, L³ is —C(O)—, L⁴ is —CH(CH₃)—, R⁶ is H, and Y is a substituted or unsubstituted quinolyl.

In another embodiment of the compounds of formula IC-3, L³ is —C(R⁴)₂— wherein R⁴ is as defined herein, L⁴ is a covalent bond, R⁶ is H, and Y is a substituted or unsubstituted quinolyl.

In another embodiment of the compounds of formula IC-3, L³ is —CH₂—, L⁴ is a covalent bond, R⁶ is H, and Y is a substituted or unsubstituted quinolyl.

In another embodiment of the compounds of formula IC-3, L³ is —CH(CH₃)—, L⁴ is a covalent bond, R⁶ is H, and Y is a substituted or unsubstituted quinolyl.

In another embodiment of the compounds of formula IC-3, L³ is —C(O)—, L⁴ is a covalent bond, R⁶ is H, and Y is a substituted or unsubstituted quinolyl.

In another embodiment of the compounds of formula IC-3, L³ is —CH₂—, L⁴ is —C(R⁴)₂—, wherein R⁴ is as defined herein, R⁶ is H, and Y is a substituted or unsubstituted indolyl.

In another embodiment of the compounds of formula IC-3, L³ is —CH(CH₃)—, L⁴ is —C(R⁴)₂— wherein R⁴ is as defined herein, R⁶ is H, and Y is a substituted or unsubstituted indolyl.

In another embodiment of the compounds of formula IC-3, L³ is —C(O)—, L⁴ is —C(R⁴)₂— wherein R⁴ is as defined herein, R⁶ is H, and Y is a substituted or unsubstituted indolyl.

In another embodiment of the compounds of formula IC-3, L³ is —C(R⁴)₂—, L⁴ is —CH(CH₃)— wherein R⁴ is as defined herein, R⁶ is H, and Y is a substituted or unsubstituted indolyl.

In another embodiment of the compounds of formula IC-3, L³ is —CH₂—, L⁴ is —CH(CH₃)—, R⁶ is H, and Y is a substituted or unsubstituted indolyl.

In another embodiment of the compounds of formula IC-3, L³ is —CH(CH₃)—, L⁴ is —CH(CH₃)—, R⁶ is H, and Y is a substituted or unsubstituted indolyl.

In another embodiment of the compounds of formula IC-3, L³ is —C(O)—, L⁴ is —CH(CH₃)—, R⁶ is H, and Y is a substituted or unsubstituted indolyl.

In another embodiment of the compounds of formula IC-3, L³ is —C(R⁴)₂— wherein R⁴ is as defined herein, L⁴ is a covalent bond, R⁶ is H, and Y is a substituted or unsubstituted indolyl.

In another embodiment of the compounds of formula IC-3, L³ is —CH₂—, L⁴ is a covalent bond, R⁶ is H, and Y is a substituted or unsubstituted indolyl.

In another embodiment of the compounds of formula IC-3, L³ is —CH(CH₃)—, L⁴ is a covalent bond, R⁶ is H, and Y is a substituted or unsubstituted indolyl.

In another embodiment of the compounds of formula IC-3, L³ is —C(O)—, L⁴ is a covalent bond, R⁶ is H, and Y is a substituted or unsubstituted indolyl.

In another embodiment of the compounds of formula IC-3, L³ is —CH₂—, L⁴ is —C(R⁴)₂—, wherein R⁴ is as defined herein, R⁶ is H, and Y is a substituted or unsubstituted thienyl.

In another embodiment of the compounds of formula IC-3, L³ is —CH(CH₃)—, L⁴ is —C(R⁴)₂— wherein R⁴ is as defined herein, R⁶ is H, and Y is a substituted or unsubstituted thienyl.

In another embodiment of the compounds of formula IC-3, L³ is —C(O)—, L⁴ is —C(R⁴)₂— wherein R⁴ is as defined herein, R⁶ is H, and Y is a substituted or unsubstituted thienyl.

In another embodiment of the compounds of formula IC-3, L³ is —C(R⁴)₂—, L⁴ is —CH(CH₃)— wherein R⁴ is as defined herein, R⁶ is H, and Y is a substituted or unsubstituted thienyl.

In another embodiment of the compounds of formula IC-3, L³ is —CH₂—, L⁴ is —CH(CH₃)—, R⁶ is H, and Y is a substituted or unsubstituted thienyl.

In another embodiment of the compounds of formula IC-3, L³ is —CH(CH₃)—, L⁴ is —CH(CH₃)—, R⁶ is H, and Y is a substituted or unsubstituted thienyl.

In another embodiment of the compounds of formula IC-3, L³ is —C(O)—, L⁴ is —CH(CH₃)—, R⁶ is H, and Y is a substituted or unsubstituted thienyl.

In another embodiment of the compounds of formula IC-3, L³ is —C(R⁴)₂— wherein R⁴ is as defined herein, L⁴ is a covalent bond, R⁶ is H, and Y is a substituted or unsubstituted thienyl.

In another embodiment of the compounds of formula IC-3, L³ is —CH₂—, L⁴ is a covalent bond, R⁶ is H, and Y is a substituted or unsubstituted thienyl.

In another embodiment of the compounds of formula IC-3, L³ is —CH(CH₃)—, L⁴ is a covalent bond, R⁶ is H, and Y is a substituted or unsubstituted thienyl.

In another embodiment of the compounds of formula IC-3, L³ is —C(O)—, L⁴ is a covalent bond, R⁶ is H, and Y is a substituted or unsubstituted thienyl.

In another embodiment of the compounds of formula IC-3, L³ is —CH₂—, L⁴ is —C(R⁴)₂—, wherein R⁴ is as defined herein, R⁶ is H, and Y is a substituted or unsubstituted purinyl.

In another embodiment of the compounds of formula IC-3, L³ is —CH(CH₃)—, L⁴ is —C(R⁴)₂— wherein R⁴ is as defined herein, R⁶ is H, and Y is a substituted or unsubstituted purinyl.

In another embodiment of the compounds of formula IC-3, L³ is —C(O)—, L⁴ is —C(R⁴)₂— wherein R⁴ is as defined herein, R⁶ is H, and Y is a substituted or unsubstituted purinyl.

In another embodiment of the compounds of formula IC-3, L³ is —C(R⁴)₂—, L⁴ is —CH(CH₃)— wherein R⁴ is as defined herein, R⁶ is H, and Y is a substituted or unsubstituted purinyl.

In another embodiment of the compounds of formula IC-3, L³ is —CH₂—, L⁴ is —CH(CH₃)—, R⁶ is H, and Y is a substituted or unsubstituted purinyl.

In another embodiment of the compounds of formula IC-3, L³ is —CH(CH₃)—, L⁴ is —CH(CH₃)—, R⁶ is H, and Y is a substituted or unsubstituted purinyl.

In another embodiment of the compounds of formula IC-3, L³ is —C(O)—, L⁴ is —CH(CH₃)—, R⁶ is H, and Y is a substituted or unsubstituted purinyl.

In another embodiment of the compounds of formula IC-3, L³ is —C(R⁴)₂— wherein R⁴ is as defined herein, L⁴ is a covalent bond, R⁶ is H, and Y is a substituted or unsubstituted purinyl.

In another embodiment of the compounds of formula IC-3, L³ is —CH₂—, L⁴ is a covalent bond, R⁶ is H, and Y is a substituted or unsubstituted purinyl.

In another embodiment of the compounds of formula IC-3, L³ is —CH(CH₃)—, L⁴ is a covalent bond, R⁶ is H, and Y is a substituted or unsubstituted purinyl.

In another embodiment of the compounds of formula IC-3, L³ is —C(O)—, L⁴ is a covalent bond, R⁶ is H, and Y is a substituted or unsubstituted purinyl.

In another embodiment of the compounds of formula IC-3, L³ is —CH₂—, L⁴ is —C(R⁴)₂—, wherein R⁴ is as defined herein, R⁶ is H, and Y is a substituted or unsubstituted furanyl.

In another embodiment of the compounds of formula IC-3, L³ is —CH(CH₃)—, L⁴ is —C(R⁴)₂— wherein R⁴ is as defined herein, R⁶ is H, and Y is a substituted or unsubstituted furanyl.

In another embodiment of the compounds of formula IC-3, L³ is —C(O)—, L⁴ is —C(R⁴)₂— wherein R⁴ is as defined herein, R⁶ is H, and Y is a substituted or unsubstituted furanyl.

In another embodiment of the compounds of formula IC-3, L³ is —C(R⁴)₂—, L⁴ is —CH(CH₃)— wherein R⁴ is as defined herein, R⁶ is H, and Y is a substituted or unsubstituted furanyl.

In another embodiment of the compounds of formula IC-3, L³ is —CH₂—, L⁴ is —CH(CH₃)—, R⁶ is H, and Y is a substituted or unsubstituted furanyl.

In another embodiment of the compounds of formula IC-3, L³ is —CH(CH₃)—, L⁴ is —CH(CH₃)—, R⁶ is H, and Y is a substituted or unsubstituted furanyl.

In another embodiment of the compounds of formula IC-3, L³ is —C(O)—, L⁴ is —CH(CH₃)—, R⁶ is H, and Y is a substituted or unsubstituted furanyl.

In another embodiment of the compounds of formula IC-3, L³ is —C(R⁴)₂— wherein R⁴ is as defined herein, L⁴ is a covalent bond, R⁶ is H, and Y is a substituted or unsubstituted furanyl.

In another embodiment of the compounds of formula IC-3, L³ is —CH₂—, L⁴ is a covalent bond, R⁶ is H, and Y is a substituted or unsubstituted furanyl.

In another embodiment of the compounds of formula IC-3, L³ is —CH(CH₃)—, L⁴ is a covalent bond, R⁶ is H, and Y is a substituted or unsubstituted furanyl.

In another embodiment of the compounds of formula IC-3, L³ is —C(O)—, L⁴ is a covalent bond, R⁶ is H, and Y is a substituted or unsubstituted furanyl.

In another embodiment of the compounds of formula IC-3, L³ is —CH₂—, L⁴ is —C(R⁴)₂—, wherein R⁴ is as defined herein, R⁶ is H, and Y is a substituted or unsubstituted pyrrolyl.

In another embodiment of the compounds of formula IC-3, L³ is —CH(CH₃)—, L⁴ is —C(R⁴)₂— wherein R⁴ is as defined herein, R⁶ is H, and Y is a substituted or unsubstituted pyrrolyl.

In another embodiment of the compounds of formula IC-3, L³ is —C(O)—, L⁴ is —C(R⁴)₂— wherein R⁴ is as defined herein, R⁶ is H, and Y is a substituted or unsubstituted pyrrolyl.

In another embodiment of the compounds of formula IC-3, L³ is —C(R⁴)₂—, L⁴ is —CH(CH₃)— wherein R⁴ is as defined herein, R⁶ is H, and Y is a substituted or unsubstituted pyrrolyl.

In another embodiment of the compounds of formula IC-3, L³ is —CH₂—, L⁴ is —CH(CH₃)—, R⁶ is H, and Y is a substituted or unsubstituted pyrrolyl.

In another embodiment of the compounds of formula IC-3, L³ is —CH(CH₃)—, L⁴ is —CH(CH₃)—, R⁶ is H, and Y is a substituted or unsubstituted pyrrolyl.

In another embodiment of the compounds of formula IC-3, L³ is —C(O)—, L⁴ is —CH(CH₃)—, R⁶ is H, and Y is a substituted or unsubstituted pyrrolyl.

In another embodiment of the compounds of formula IC-3, L³ is —C(R⁴)₂— wherein R⁴ is as defined herein, L⁴ is a covalent bond, R⁶ is H, and Y is a substituted or unsubstituted pyrrolyl.

In another embodiment of the compounds of formula IC-3, L³ is —CH₂—, L⁴ is a covalent bond, R⁶ is H, and Y is a substituted or unsubstituted pyrrolyl.

In another embodiment of the compounds of formula IC-3, L³ is —CH(CH₃)—, L⁴ is a covalent bond, R⁶ is H, and Y is a substituted or unsubstituted pyrrolyl.

In another embodiment of the compounds of formula IC-3, L³ is —C(O)—, L⁴ is a covalent bond, R⁶ is H, and Y is a substituted or unsubstituted pyrrolyl.

In another embodiment of the compounds of formula IC-3, L³ is —CH₂—, L⁴ is —C(R⁴)₂—, wherein R⁴ is as defined herein, R⁶ is H, and Y is alkoxyalkyl.

In another embodiment of the compounds of formula IC-3, L³ is —CH(CH₃)—, L⁴ is —C(R⁴)₂— wherein R⁴ is as defined herein, R⁶ is H, and Y is alkoxyalkyl.

In another embodiment of the compounds of formula IC-3, L³ is —C(O)—, L⁴ is —C(R⁴)₂— wherein R⁴ is as defined herein, R⁶ is H, and Y is alkoxyalkyl.

In another embodiment of the compounds of formula IC-3, L³ is —C(R⁴)₂—, L⁴ is —CH(CH₃)— wherein R⁴ is as defined herein, R⁶ is H, and Y is alkoxyalkyl.

In another embodiment of the compounds of formula IC-3, L³ is —CH₂—, L⁴ is —CH(CH₃)—, R⁶ is H, and Y is alkoxyalkyl.

In another embodiment of the compounds of formula IC-3, L³ is —CH(CH₃)—, L⁴ is —CH(CH₃)—, R⁶ is H, and Y is alkoxyalkyl.

In another embodiment of the compounds of formula IC-3, L³ is —C(O)—, L⁴ is —CH(CH₃)—, R⁶ is H, and Y is alkoxyalkyl.

In another embodiment of the compounds of formula IC-3, L³ is —C(R⁴)₂— wherein R⁴ is as defined herein, L⁴ is a covalent bond, R⁶ is H, and Y is alkoxyalkyl.

In another embodiment of the compounds of formula IC-3, L³ is —CH₂—, L⁴ is a covalent bond, R⁶ is H, and Y is alkoxyalkyl.

In another embodiment of the compounds of formula IC-3, L³ is —CH(CH₃)—, L⁴ is a covalent bond, R⁶ is H, and Y is alkoxyalkyl.

In another embodiment of the compounds of formula IC-3, L³ is —C(O)—, L⁴ is a covalent bond, R⁶ is H, and Y is alkoxyalkyl.

In another embodiment of the compounds of formula IC-3, L³ is —C(R⁴)₂—, L⁴ is —C(R⁴)₂—, wherein R⁴ is as defined herein, R⁶ is alkyl, and Y is a substituted or unsubstituted aryl.

In another embodiment of the compounds of formula IC-3, L³ is —CH₂—, L⁴ is —C(R⁴)₂—, wherein R⁴ is as defined herein, R⁶ is alkyl, and Y is a substituted or unsubstituted aryl.

In another embodiment of the compounds of formula IC-3, L³ is —CH(CH₃)—, L⁴ is —C(R⁴)₂— wherein R⁴ is as defined herein, R⁶ is alkyl, and Y is a substituted or unsubstituted aryl.

In another embodiment of the compounds of formula IC-3, L³ is —C(O)—, L⁴ is —C(R⁴)₂— wherein R⁴ is as defined herein, R⁶ is alkyl, and Y is a substituted or unsubstituted aryl.

In another embodiment of the compounds of formula IC-3, L³ is —C(R⁴)₂—, L⁴ is —CH(CH₃)— wherein R⁴ is as defined herein, R⁶ is alkyl, and Y is a substituted or unsubstituted aryl.

In another embodiment of the compounds of formula IC-3, L³ is —CH₂—, L⁴ is —CH(CH₃)—, R⁶ is alkyl, and Y is a substituted or unsubstituted aryl.

In another embodiment of the compounds of formula IC-3, L³ is —CH(CH₃)—, L⁴ is —CH(CH₃)—, R⁶ is alkyl, and Y is a substituted or unsubstituted aryl.

In another embodiment of the compounds of formula IC-3, L³ is —C(O)—, L⁴ is —CH(CH₃)—, R⁶ is alkyl, and Y is a substituted or unsubstituted aryl.

In another embodiment of the compounds of formula IC-3, L³ is —C(R⁴)₂— wherein R⁴ is as defined herein, L⁴ is a covalent bond, R⁶ is alkyl, and Y is a substituted or unsubstituted aryl.

In another embodiment of the compounds of formula IC-3, L³ is —CH₂—, L⁴ is a covalent bond, R⁶ is alkyl, and Y is a substituted or unsubstituted aryl.

In another embodiment of the compounds of formula IC-3, L³ is —CH(CH₃)—, L⁴ is a covalent bond, R⁶ is alkyl, and Y is a substituted or unsubstituted aryl.

In another embodiment of the compounds of formula IC-3, L³ is —C(O)—, L⁴ is a covalent bond, R⁶ is alkyl, and Y is a substituted or unsubstituted aryl.

In another embodiment of the compounds of formula IC-3, L³ is —C(R⁴)₂—, L⁴ is —C(R⁴)₂—, wherein R⁴ is as defined herein, R⁶ is alkyl, and Y is a substituted or unsubstituted phenyl.

In another embodiment of the compounds of formula IC-3, L³ is —CH₂—, L⁴ is —C(R⁴)₂—, wherein R⁴ is as defined herein, R⁶ is alkyl, and Y is a substituted or unsubstituted phenyl.

In another embodiment of the compounds of formula IC-3, L³ is —CH(CH₃)—, L⁴ is —C(R⁴)₂— wherein R⁴ is as defined herein, R⁶ is alkyl, and Y is a substituted or unsubstituted phenyl.

In another embodiment of the compounds of formula IC-3, L³ is —C(O)—, L⁴ is —C(R⁴)₂— wherein R⁴ is as defined herein, R⁶ is alkyl, and Y is a substituted or unsubstituted phenyl.

In another embodiment of the compounds of formula IC-3, L³ is —C(R⁴)₂—, L⁴ is —CH(CH₃)— wherein R⁴ is as defined herein, R⁶ is alkyl, and Y is a substituted or unsubstituted phenyl.

In another embodiment of the compounds of formula IC-3, L³ is —CH₂—, L⁴ is —CH(CH₃)—, R⁶ is alkyl, and Y is a substituted or unsubstituted phenyl.

In another embodiment of the compounds of formula IC-3, L³ is —CH(CH₃)—, L⁴ is —CH(CH₃)—, R⁶ is alkyl, and Y is a substituted or unsubstituted phenyl.

In another embodiment of the compounds of formula IC-3, L³ is —C(O)—, L⁴ is —CH(CH₃)—, R⁶ is alkyl, and Y is a substituted or unsubstituted phenyl.

In another embodiment of the compounds of formula IC-3, L³ is —C(R⁴)₂— wherein R⁴ is as defined herein, L⁴ is a covalent bond, R⁶ is alkyl, and Y is a substituted or unsubstituted phenyl.

In another embodiment of the compounds of formula IC-3, L³ is —CH₂—, L⁴ is a covalent bond, R⁶ is alkyl, and Y is a substituted or unsubstituted phenyl.

In another embodiment of the compounds of formula IC-3, L³ is —CH(CH₃)—, L⁴ is a covalent bond, R⁶ is alkyl, and Y is a substituted or unsubstituted phenyl.

In another embodiment of the compounds of formula IC-3, L³ is —C(O)—, L⁴ is a covalent bond, R⁶ is alkyl, and Y is a substituted or unsubstituted phenyl.

In another embodiment of the compounds of formula IC-3, L³ is —C(R⁴)₂—, L⁴ is —C(R⁴)₂—, wherein R⁴ is as defined herein, R⁶ is alkyl, and Y is a substituted or unsubstituted heteroaryl.

In another embodiment of the compounds of formula IC-3, L³ is —CH₂—, L⁴ is —C(R⁴)₂—, wherein R⁴ is as defined herein, R⁶ is alkyl, and Y is a substituted or unsubstituted heteroaryl.

In another embodiment of the compounds of formula IC-3, L³ is —CH(CH₃)—, L⁴ is —C(R⁴)₂— wherein R⁴ is as defined herein, R⁶ is alkyl, and Y is a substituted or unsubstituted heteroaryl.

In another embodiment of the compounds of formula IC-3, L³ is —C(O)—, L⁴ is —C(R⁴)₂— wherein R⁴ is as defined herein, R⁶ is alkyl, and Y is a substituted or unsubstituted heteroaryl.

In another embodiment of the compounds of formula IC-3, L³ is —C(R⁴)₂—, L⁴ is —CH(CH₃)— wherein R⁴ is as defined herein, R⁶ is alkyl, and Y is a substituted or unsubstituted heteroaryl.

In another embodiment of the compounds of formula IC-3, L³ is —CH₂—, L⁴ is —CH(CH₃)—, R⁶ is alkyl, and Y is a substituted or unsubstituted heteroaryl.

In another embodiment of the compounds of formula IC-3, L³ is —CH(CH₃)—, L⁴ is —CH(CH₃)—, R⁶ is alkyl, and Y is a substituted or unsubstituted heteroaryl.

In another embodiment of the compounds of formula IC-3, L³ is —C(O)—, L⁴ is —CH(CH₃)—, R⁶ is alkyl, and Y is a substituted or unsubstituted heteroaryl.

In another embodiment of the compounds of formula IC-3, L³ is —C(R⁴)₂— wherein R⁴ is as defined herein, L⁴ is a covalent bond, R⁶ is alkyl, and Y is a substituted or unsubstituted heteroaryl.

In another embodiment of the compounds of formula IC-3, L³ is —CH₂—, L⁴ is a covalent bond, R⁶ is alkyl, and Y is a substituted or unsubstituted heteroaryl.

In another embodiment of the compounds of formula IC-3, L³ is —CH(CH₃)—, L⁴ is a covalent bond, R⁶ is alkyl, and Y is a substituted or unsubstituted heteroaryl.

In another embodiment of the compounds of formula IC-3, L³ is —C(O)—, L⁴ is a covalent bond, R⁶ is alkyl, and Y is a substituted or unsubstituted heteroaryl.

In another embodiment of the compounds of formula IC-3, L³ is —CH₂—, L⁴ is —C(R⁴)₂—, wherein R⁴ is as defined herein, R⁶ is alkyl, and Y is a substituted or unsubstituted pyridyl.

In another embodiment of the compounds of formula IC-3, L³ is —CH(CH₃)—, L⁴ is —C(R⁴)₂— wherein R⁴ is as defined herein, R⁶ is alkyl, and Y is a substituted or unsubstituted pyridyl.

In another embodiment of the compounds of formula IC-3, L³ is —C(O)—, L⁴ is —C(R⁴)₂— wherein R⁴ is as defined herein, R⁶ is alkyl, and Y is a substituted or unsubstituted pyridyl.

In another embodiment of the compounds of formula IC-3, L³ is —C(R⁴)₂—, L⁴ is —CH(CH₃)— wherein R⁴ is as defined herein, R⁶ is alkyl, and Y is a substituted or unsubstituted pyridyl.

In another embodiment of the compounds of formula IC-3, L³ is —CH₂—, L⁴ is —CH(CH₃)—, R⁶ is alkyl, and Y is a substituted or unsubstituted pyridyl.

In another embodiment of the compounds of formula IC-3, L³ is —CH(CH₃)—, L⁴ is —CH(CH₃)—, R⁶ is alkyl, and Y is a substituted or unsubstituted pyridyl.

In another embodiment of the compounds of formula IC-3, L³ is —C(O)—, L⁴ is —CH(CH₃)—, R⁶ is alkyl, and Y is a substituted or unsubstituted pyridyl.

In another embodiment of the compounds of formula IC-3, L³ is —C(R⁴)₂— wherein R⁴ is as defined herein, L⁴ is a covalent bond, R⁶ is alkyl, and Y is a substituted or unsubstituted pyridyl.

In another embodiment of the compounds of formula IC-3, L³ is —CH₂—, L⁴ is a covalent bond, R⁶ is alkyl, and Y is a substituted or unsubstituted pyridyl.

In another embodiment of the compounds of formula IC-3, L³ is —CH(CH₃)—, L⁴ is a covalent bond, R⁶ is alkyl, and Y is a substituted or unsubstituted pyridyl.

In another embodiment of the compounds of formula IC-3, L³ is —C(O)—, L⁴ is a covalent bond, R⁶ is alkyl, and Y is a substituted or unsubstituted pyridyl.

In another embodiment of the compounds of formula IC-3, L³ is —CH₂—, L⁴ is —C(R⁴)₂—, wherein R⁴ is as defined herein, R⁶ is alkyl, and Y is a substituted or unsubstituted pyrimidyl.

In another embodiment of the compounds of formula IC-3, L³ is —CH(CH₃)—, L⁴ is —C(R⁴)₂— wherein R⁴ is as defined herein, R⁶ is alkyl, and Y is a substituted or unsubstituted pyrimidyl.

In another embodiment of the compounds of formula IC-3, L³ is —C(O)—, L⁴ is —C(R⁴)₂— wherein R⁴ is as defined herein, R⁶ is alkyl, and Y is a substituted or unsubstituted pyrimidyl.

In another embodiment of the compounds of formula IC-3, L³ is —C(R⁴)₂—, L⁴ is —CH(CH₃)— wherein R⁴ is as defined herein, R⁶ is alkyl, and Y is a substituted or unsubstituted pyrimidyl.

In another embodiment of the compounds of formula IC-3, L³ is —CH₂—, L⁴ is —CH(CH₃)—, R⁶ is alkyl, and Y is a substituted or unsubstituted pyrimidyl.

In another embodiment of the compounds of formula IC-3, L³ is —CH(CH₃)—, L⁴ is —CH(CH₃)—, R⁶ is alkyl, and Y is a substituted or unsubstituted pyrimidyl.

In another embodiment of the compounds of formula IC-3, L³ is —C(O)—, L⁴ is —CH(CH₃)—, R⁶ is alkyl, and Y is a substituted or unsubstituted pyrimidyl.

In another embodiment of the compounds of formula IC-3, L³ is —C(R⁴)₂— wherein R⁴ is as defined herein, L⁴ is a covalent bond, R⁶ is alkyl, and Y is a substituted or unsubstituted pyrimidyl.

In another embodiment of the compounds of formula IC-3, L³ is —CH₂—, L⁴ is a covalent bond, R⁶ is alkyl, and Y is a substituted or unsubstituted pyrimidyl.

In another embodiment of the compounds of formula IC-3, L³ is —CH(CH₃)—, L⁴ is a covalent bond, R⁶ is alkyl, and Y is a substituted or unsubstituted pyrimidyl.

In another embodiment of the compounds of formula IC-3, L³ is —C(O)—, L⁴ is a covalent bond, R⁶ is alkyl, and Y is a substituted or unsubstituted pyrimidyl.

In another embodiment of the compounds of formula IC-3, L³ is —CH₂—, L⁴ is —C(R⁴)₂—, wherein R⁴ is as defined herein, R⁶ is alkyl, and Y is a substituted or unsubstituted quinolyl.

In another embodiment of the compounds of formula IC-3, L³ is —CH(CH₃)—, L⁴ is —C(R⁴)₂— wherein R⁴ is as defined herein, R⁶ is alkyl, and Y is a substituted or unsubstituted quinolyl.

In another embodiment of the compounds of formula IC-3, L³ is —C(O)—, L⁴ is —C(R⁴)₂— wherein R⁴ is as defined herein, R⁶ is alkyl, and Y is a substituted or unsubstituted quinolyl.

In another embodiment of the compounds of formula IC-3, L³ is —C(R⁴)₂—, L⁴ is —CH(CH₃)— wherein R⁴ is as defined herein, R⁶ is alkyl, and Y is a substituted or unsubstituted quinolyl.

In another embodiment of the compounds of formula IC-3, L³ is —CH₂—, L⁴ is —CH(CH₃)—, R⁶ is alkyl, and Y is a substituted or unsubstituted quinolyl.

In another embodiment of the compounds of formula IC-3, L³ is —CH(CH₃)—, L⁴ is —CH(CH₃)—, R⁶ is alkyl, and Y is a substituted or unsubstituted quinolyl.

In another embodiment of the compounds of formula IC-3, L³ is —C(O)—, L⁴ is —CH(CH₃)—, R⁶ is alkyl, and Y is a substituted or unsubstituted quinolyl.

In another embodiment of the compounds of formula IC-3, L³ is —C(R⁴)₂— wherein R⁴ is as defined herein, L⁴ is a covalent bond, R⁶ is alkyl, and Y is a substituted or unsubstituted quinolyl.

In another embodiment of the compounds of formula IC-3, L³ is —CH₂—, L⁴ is a covalent bond, R⁶ is alkyl, and Y is a substituted or unsubstituted quinolyl.

In another embodiment of the compounds of formula IC-3, L³ is —CH(CH₃)—, L⁴ is a covalent bond, R⁶ is alkyl, and Y is a substituted or unsubstituted quinolyl.

In another embodiment of the compounds of formula IC-3, L³ is —C(O)—, L⁴ is a covalent bond, R⁶ is alkyl, and Y is a substituted or unsubstituted quinolyl.

In another embodiment of the compounds of formula IC-3, L³ is —CH₂—, L¹ is —C(R⁴)₂—, wherein R⁴ is as defined herein, R⁶ is alkyl, and Y is a substituted or unsubstituted indolyl.

In another embodiment of the compounds of formula IC-3, L³ is —CH(CH₃)—, L⁴ is —C(R⁴)₂— wherein R⁴ is as defined herein, R⁶ is alkyl, and Y is a substituted or unsubstituted indolyl.

In another embodiment of the compounds of formula IC-3, L³ is —C(O)—, L⁴ is —C(R⁴)₂— wherein R⁴ is as defined herein, R⁶ is alkyl, and Y is a substituted or unsubstituted indolyl.

In another embodiment of the compounds of formula IC-3, L³ is —C(R⁴)₂—, L⁴ is —CH(CH₃)— wherein R⁴ is as defined herein, R⁶ is alkyl, and Y is a substituted or unsubstituted indolyl.

In another embodiment of the compounds of formula IC-3, L³ is —CH₂—, L⁴ is —CH(CH₃)—, R⁶ is alkyl, and Y is a substituted or unsubstituted indolyl.

In another embodiment of the compounds of formula IC-3, L³ is —CH(CH₃)—, L⁴ is —CH(CH₃)—, R⁶ is alkyl, and Y is a substituted or unsubstituted indolyl.

In another embodiment of the compounds of formula IC-3, L³ is —C(O)—, L⁴ is —CH(CH₃)—, R⁶ is alkyl, and Y is a substituted or unsubstituted indolyl.

In another embodiment of the compounds of formula IC-3, L³ is —C(R⁴)₂— wherein R⁴ is as defined herein, L⁴ is a covalent bond, R⁶ is alkyl, and Y is a substituted or unsubstituted indolyl.

In another embodiment of the compounds of formula IC-3, L³ is —CH₂—, L⁴ is a covalent bond, R⁶ is alkyl, and Y is a substituted or unsubstituted indolyl.

In another embodiment of the compounds of formula IC-3, L³ is —CH(CH₃)—, L⁴ is a covalent bond, R⁶ is alkyl, and Y is a substituted or unsubstituted indolyl.

In another embodiment of the compounds of formula IC-3, L³ is —C(O)—, L⁴ is a covalent bond, R⁶ is alkyl, and Y is a substituted or unsubstituted indolyl.

In another embodiment of the compounds of formula IC-3, L³ is —CH₂—, L⁴ is —C(R⁴)₂—, wherein R⁴ is as defined herein, R⁶ is alkyl, and Y is a substituted or unsubstituted thienyl.

In another embodiment of the compounds of formula IC-3, L³ is —CH(CH₃)—, L⁴ is —C(R⁴)₂— wherein R⁴ is as defined herein, R⁶ is alkyl, and Y is a substituted or unsubstituted thienyl.

In another embodiment of the compounds of formula IC-3, L³ is —C(O)—, L⁴ is —C(R⁴)₂— wherein R⁴ is as defined herein, R⁶ is alkyl, and Y is a substituted or unsubstituted thienyl.

In another embodiment of the compounds of formula IC-3, L³ is —C(R⁴)₂—, L⁴ is —CH(CH₃)— wherein R⁴ is as defined herein, R⁶ is alkyl, and Y is a substituted or unsubstituted thienyl.

In another embodiment of the compounds of formula IC-3, L³ is —CH₂—, L⁴ is —CH(CH₃)—, R⁶ is alkyl, and Y is a substituted or unsubstituted thienyl.

In another embodiment of the compounds of formula IC-3, L³ is —CH(CH₃)—, L⁴ is —CH(CH₃)—, R⁶ is alkyl, and Y is a substituted or unsubstituted thienyl.

In another embodiment of the compounds of formula IC-3, L³ is —C(O)—, L⁴ is —CH(CH₃)—, R⁶ is alkyl, and Y is a substituted or unsubstituted thienyl.

In another embodiment of the compounds of formula IC-3, L³ is —C(R⁴)₂— wherein R⁴ is as defined herein, L⁴ is a covalent bond, R⁶ is alkyl, and Y is a substituted or unsubstituted thienyl.

In another embodiment of the compounds of formula IC-3, L³ is —CH₂—, L⁴ is a covalent bond, R⁶ is alkyl, and Y is a substituted or unsubstituted thienyl.

In another embodiment of the compounds of formula IC-3, L³ is —CH(CH₃)—, L⁴ is a covalent bond, R⁶ is alkyl, and Y is a substituted or unsubstituted thienyl.

In another embodiment of the compounds of formula IC-3, L³ is —C(O)—, L⁴ is a covalent bond, R⁶ is alkyl, and Y is a substituted or unsubstituted thienyl.

In another embodiment of the compounds of formula IC-3, L³ is —CH₂—, L⁴ is —C(R⁴)₂—, wherein R⁴ is as defined herein, R⁶ is alkyl, and Y is a substituted or unsubstituted purinyl.

In another embodiment of the compounds of formula IC-3, L³ is —CH(CH₃)—, L⁴ is —C(R⁴)₂— wherein R⁴ is as defined herein, R⁶ is alkyl, and Y is a substituted or unsubstituted purinyl.

In another embodiment of the compounds of formula IC-3, L³ is —C(O)—, L⁴ is —C(R⁴)₂— wherein R⁴ is as defined herein, R⁶ is alkyl, and Y is a substituted or unsubstituted purinyl.

In another embodiment of the compounds of formula IC-3, L³ is —C(R⁴)₂—, L⁴ is —CH(CH₃)— wherein R⁴ is as defined herein, R⁶ is alkyl, and Y is a substituted or unsubstituted purinyl.

In another embodiment of the compounds of formula IC-3, L³ is —CH₂—, L⁴ is —CH(CH₃)—, R⁶ is alkyl, and Y is a substituted or unsubstituted purinyl.

In another embodiment of the compounds of formula IC-3, L³ is —CH(CH₃)—, L⁴ is —CH(CH₃)—, R⁶ is alkyl, and Y is a substituted or unsubstituted purinyl.

In another embodiment of the compounds of formula IC-3, L³ is —C(O)—, L⁴ is —CH(CH₃)—, R⁶ is alkyl, and Y is a substituted or unsubstituted purinyl.

In another embodiment of the compounds of formula IC-3, L³ is —C(R⁴)₂— wherein R⁴ is as defined herein, L⁴ is a covalent bond, R⁶ is alkyl, and Y is a substituted or unsubstituted purinyl.

In another embodiment of the compounds of formula IC-3, L³ is —CH₂—, L⁴ is a covalent bond, R⁶ is alkyl, and Y is a substituted or unsubstituted purinyl.

In another embodiment of the compounds of formula IC-3, L³ is —CH(CH₃)—, L⁴ is a covalent bond, R⁶ is alkyl, and Y is a substituted or unsubstituted purinyl.

In another embodiment of the compounds of formula IC-3, L³ is —C(O)—, L⁴ is a covalent bond, R⁶ is alkyl, and Y is a substituted or unsubstituted purinyl.

In another embodiment of the compounds of formula IC-3, L³ is —CH₂—, L⁴ is —C(R⁴)₂—, wherein R⁴ is as defined herein, R⁶ is alkyl, and Y is a substituted or unsubstituted furanyl.

In another embodiment of the compounds of formula IC-3, L³ is —CH(CH₃)—, L⁴ is —C(R⁴)₂— wherein R⁴ is as defined herein, R⁶ is alkyl, and Y is a substituted or unsubstituted furanyl.

In another embodiment of the compounds of formula IC-3, L³ is —C(O)—, L⁴ is —C(R⁴)₂— wherein R⁴ is as defined herein, R⁶ is alkyl, and Y is a substituted or unsubstituted furanyl.

In another embodiment of the compounds of formula IC-3, L³ is —C(R⁴)₂—, L⁴ is —CH(CH₃)— wherein R⁴ is as defined herein, R⁶ is alkyl, and Y is a substituted or unsubstituted furanyl.

In another embodiment of the compounds of formula IC-3, L³ is —CH₂—, L⁴ is —CH(CH₃)—, R⁶ is alkyl, and Y is a substituted or unsubstituted furanyl.

In another embodiment of the compounds of formula IC-3, L³ is —CH(CH₃)—, L⁴ is —CH(CH₃)—, R⁶ is alkyl, and Y is a substituted or unsubstituted furanyl.

In another embodiment of the compounds of formula IC-3, L³ is —C(O)—, L⁴ is —CH(CH₃)—, R⁶ is alkyl, and Y is a substituted or unsubstituted furanyl.

In another embodiment of the compounds of formula IC-3, L³ is —C(R⁴)₂— wherein R⁴ is as defined herein, L⁴ is a covalent bond, R⁶ is alkyl, and Y is a substituted or unsubstituted furanyl.

In another embodiment of the compounds of formula IC-3, L³ is —CH₂—, L⁴ is a covalent bond, R⁶ is alkyl, and Y is a substituted or unsubstituted furanyl.

In another embodiment of the compounds of formula IC-3, L³ is —CH(CH₃)—, L⁴ is a covalent bond, R⁶ is alkyl, and Y is a substituted or unsubstituted furanyl.

In another embodiment of the compounds of formula IC-3, L³ is —C(O)—, L⁴ is a covalent bond, R⁶ is alkyl, and Y is a substituted or unsubstituted furanyl.

In another embodiment of the compounds of formula IC-3, L³ is —CH₂—, L⁴ is —C(R⁴)₂—, wherein R⁴ is as defined herein, R⁶ is alkyl, and Y is a substituted or unsubstituted pyrrolyl.

In another embodiment of the compounds of formula IC-3, L³ is —CH(CH₃)—, L⁴ is —C(R⁴)₂— wherein R⁴ is as defined herein, R⁶ is alkyl, and Y is a substituted or unsubstituted pyrrolyl.

In another embodiment of the compounds of formula IC-3, L³ is —C(O)—, L⁴ is —C(R⁴)₂— wherein R⁴ is as defined herein, R⁶ is alkyl, and Y is a substituted or unsubstituted pyrrolyl.

In another embodiment of the compounds of formula IC-3, L³ is —C(R⁴)₂—, L⁴ is —CH(CH₃)— wherein R⁴ is as defined herein, R⁶ is alkyl, and Y is a substituted or unsubstituted pyrrolyl.

In another embodiment of the compounds of formula IC-3, L³ is —CH₂—, L⁴ is —CH(CH₃)—, R⁶ is alkyl, and Y is a substituted or unsubstituted pyrrolyl.

In another embodiment of the compounds of formula IC-3, L³ is —CH(CH₃)—, L⁴ is —CH(CH₃)—, R⁶ is alkyl, and Y is a substituted or unsubstituted pyrrolyl.

In another embodiment of the compounds of formula IC-3, L³ is —C(O)—, L⁴ is —CH(CH₃)—, R⁶ is alkyl, and Y is a substituted or unsubstituted pyrrolyl.

In another embodiment of the compounds of formula IC-3, L³ is —C(R⁴)₂— wherein R⁴ is as defined herein, L⁴ is a covalent bond, R⁶ is alkyl, and Y is a substituted or unsubstituted pyrrolyl.

In another embodiment of the compounds of formula IC-3, L³ is —CH₂—, L⁴ is a covalent bond, R⁶ is alkyl, and Y is a substituted or unsubstituted pyrrolyl.

In another embodiment of the compounds of formula IC-3, L³ is —CH(CH₃)—, L⁴ is a covalent bond, R⁶ is alkyl, and Y is a substituted or unsubstituted pyrrolyl.

In another embodiment of the compounds of formula IC-3, L³ is —C(O)—, L⁴ is a covalent bond, R⁶ is alkyl, and Y is a substituted or unsubstituted pyrrolyl.

In another embodiment of the compounds of formula IC-3, L³ is —CH₂—, L⁴ is —C(R⁴)₂—, wherein R⁴ is as defined herein, R⁶ is alkyl, and Y is alkoxyalkyl.

In another embodiment of the compounds of formula IC-3, L³ is —CH(CH₃)—, L⁴ is —C(R⁴)₂— wherein R⁴ is as defined herein, R⁶ is alkyl, and Y is alkoxyalkyl.

In another embodiment of the compounds of formula IC-3, L³ is —C(O)—, L⁴ is —C(R⁴)₂— wherein R⁴ is as defined herein, R⁶ is alkyl, and Y is alkoxyalkyl.

In another embodiment of the compounds of formula IC-3, L³ is —C(R⁴)₂—, L⁴ is —CH(CH₃)— wherein R⁴ is as defined herein, R⁶ is alkyl, and Y is alkoxyalkyl.

In another embodiment of the compounds of formula IC-3, L³ is —CH₂—, L⁴ is —CH(CH₃)—, R⁶ is alkyl, and Y is alkoxyalkyl.

In another embodiment of the compounds of formula IC-3, L³ is —CH(CH₃)—, L⁴ is —CH(CH₃)—, R⁶ is alkyl, and Y is alkoxyalkyl.

In another embodiment of the compounds of formula IC-3, L³ is —C(O)—, L⁴ is —CH(CH₃)—, R⁶ is alkyl, and Y is alkoxyalkyl.

In another embodiment of the compounds of formula IC-3, L³ is —C(R⁴)₂— wherein R⁴ is as defined herein, L⁴ is a covalent bond, R⁶ is alkyl, and Y is alkoxyalkyl.

In another embodiment of the compounds of formula IC-3, L³ is —CH₂—, L⁴ is a covalent bond, R⁶ is alkyl, and Y is alkoxyalkyl.

In another embodiment of the compounds of formula IC-3, L³ is —CH(CH₃)—, L⁴ is a covalent bond, R⁶ is alkyl, and Y is alkoxyalkyl.

In another embodiment of the compounds of formula IC-3, L³ is —C(O)—, L⁴ is a covalent bond, R⁶ is alkyl, and Y is alkoxyalkyl.

In another embodiment of the compounds of formula IC-3, L³ is —C(R⁴)₂—, L⁴ is —C(R⁴)₂—, wherein R⁴ is as defined herein, R⁶ is methyl, and Y is a substituted or unsubstituted aryl.

In another embodiment of the compounds of formula IC-3, L³ is —CH₂—, L⁴ is —C(R⁴)₂—, wherein R⁴ is as defined herein, R⁶ is methyl, and Y is a substituted or unsubstituted aryl.

In another embodiment of the compounds of formula IC-3, L³ is —CH(CH₃)—, L⁴ is —C(R⁴)₂— wherein R⁴ is as defined herein, R⁶ is methyl, and Y is a substituted or unsubstituted aryl.

In another embodiment of the compounds of formula IC-3, L³ is —C(O)—, L⁴ is —C(R⁴)₂— wherein R⁴ is as defined herein, R⁶ is methyl, and Y is a substituted or unsubstituted aryl.

In another embodiment of the compounds of formula IC-3, L³ is —C(R⁴)₂—, L⁴ is —CH(CH₃)— wherein R⁴ is as defined herein, R⁶ is methyl, and Y is a substituted or unsubstituted aryl.

In another embodiment of the compounds of formula IC-3, L³ is —CH₂—, L⁴ is —CH(CH₃)—, R⁶ is methyl, and Y is a substituted or unsubstituted aryl.

In another embodiment of the compounds of formula IC-3, L³ is —CH(CH₃)—, L⁴ is —CH(CH₃)—, R⁶ is methyl, and Y is a substituted or unsubstituted aryl.

In another embodiment of the compounds of formula IC-3, L³ is —C(O)—, L⁴ is —CH(CH₃)—, R⁶ is methyl, and Y is a substituted or unsubstituted aryl.

In another embodiment of the compounds of formula IC-3, L³ is —C(R⁴)₂— wherein R⁴ is as defined herein, L⁴ is a covalent bond, R⁶ is methyl, and Y is a substituted or unsubstituted aryl.

In another embodiment of the compounds of formula IC-3, L³ is —CH₂—, L⁴ is a covalent bond, R⁶ is methyl, and Y is a substituted or unsubstituted aryl.

In another embodiment of the compounds of formula IC-3, L³ is —CH(CH₃)—, L⁴ is a covalent bond, R⁶ is methyl, and Y is a substituted or unsubstituted aryl.

In another embodiment of the compounds of formula IC-3, L³ is —C(O)—, L⁴ is a covalent bond, R⁶ is methyl, and Y is a substituted or unsubstituted aryl.

In another embodiment of the compounds of formula IC-3, L³ is —C(R⁴)₂—, L⁴ is —C(R⁴)₂—, wherein R⁴ is as defined herein, R⁶ is methyl, and Y is a substituted or unsubstituted phenyl.

In another embodiment of the compounds of formula IC-3, L³ is —CH₂—, L⁴ is —C(R⁴)₂—, wherein R⁴ is as defined herein, R⁶ is methyl, and Y is a substituted or unsubstituted phenyl.

In another embodiment of the compounds of formula IC-3, L³ is —CH(CH₃)—, L⁴ is —C(R⁴)₂— wherein R⁴ is as defined herein, R⁶ is methyl, and Y is a substituted or unsubstituted phenyl.

In another embodiment of the compounds of formula IC-3, L³ is —C(O)—, L⁴ is —C(R⁴)₂— wherein R⁴ is as defined herein, R⁶ is methyl, and Y is a substituted or unsubstituted phenyl.

In another embodiment of the compounds of formula IC-3, L³ is —C(R⁴)₂—, L⁴ is —CH(CH₃)— wherein R⁴ is as defined herein, R⁶ is methyl, and Y is a substituted or unsubstituted phenyl.

In another embodiment of the compounds of formula IC-3, L³ is —CH₂—, L⁴ is —CH(CH₃)—, R⁶ is methyl, and Y is a substituted or unsubstituted phenyl.

In another embodiment of the compounds of formula IC-3, L³ is —CH(CH₃)—, L⁴ is —CH(CH₃)—, R⁶ is methyl, and Y is a substituted or unsubstituted phenyl.

In another embodiment of the compounds of formula IC-3, L³ is —C(O)—, L⁴ is —CH(CH₃)—, R⁶ is methyl, and Y is a substituted or unsubstituted phenyl.

In another embodiment of the compounds of formula IC-3, L³ is —C(R⁴)₂— wherein R⁴ is as defined herein, L⁴ is a covalent bond, R⁶ is methyl, and Y is a substituted or unsubstituted phenyl.

In another embodiment of the compounds of formula IC-3, L³ is —CH₂—, L⁴ is a covalent bond, R⁶ is methyl, and Y is a substituted or unsubstituted phenyl.

In another embodiment of the compounds of formula IC-3, L³ is —CH(CH₃)—, L⁴ is a covalent bond, R⁶ is methyl, and Y is a substituted or unsubstituted phenyl.

In another embodiment of the compounds of formula IC-3, L³ is —C(O)—, L⁴ is a covalent bond, R⁶ is methyl, and Y is a substituted or unsubstituted phenyl.

In another embodiment of the compounds of formula IC-3, L³ is —C(R⁴)₂—, L⁴ is —C(R⁴)₂—, wherein R⁴ is as defined herein, R⁶ is methyl, and Y is a substituted or unsubstituted heteroaryl.

In another embodiment of the compounds of formula IC-3, L³ is —CH₂—, L⁴ is —C(R⁴)₂—, wherein R⁴ is as defined herein, R⁶ is methyl, and Y is a substituted or unsubstituted heteroaryl.

In another embodiment of the compounds of formula IC-3, L³ is —CH(CH₃)—, L⁴ is —C(R⁴)₂— wherein R⁴ is as defined herein, R⁶ is methyl, and Y is a substituted or unsubstituted heteroaryl.

In another embodiment of the compounds of formula IC-3, L³ is —C(O)—, L⁴ is —C(R⁴)₂— wherein R⁴ is as defined herein, R⁶ is methyl, and Y is a substituted or unsubstituted heteroaryl.

In another embodiment of the compounds of formula IC-3, L³ is —C(R⁴)₂—, L⁴ is —CH(CH₃)— wherein R⁴ is as defined herein, R⁶ is methyl, and Y is a substituted or unsubstituted heteroaryl.

In another embodiment of the compounds of formula IC-3, L³ is —CH₂—, L⁴ is —CH(CH₃)—, R⁶ is methyl, and Y is a substituted or unsubstituted heteroaryl.

In another embodiment of the compounds of formula IC-3, L³ is —CH(CH₃)—, L⁴ is —CH(CH₃)—, R⁶ is methyl, and Y is a substituted or unsubstituted heteroaryl.

In another embodiment of the compounds of formula IC-3, L³ is —C(O)—, L⁴ is —CH(CH₃)—, R⁶ is methyl, and Y is a substituted or unsubstituted heteroaryl.

In another embodiment of the compounds of formula IC-3, L³ is —C(R⁴)₂— wherein R⁴ is as defined herein, L⁴ is a covalent bond, R⁶ is methyl, and Y is a substituted or unsubstituted heteroaryl.

In another embodiment of the compounds of formula IC-3, L³ is —CH₂—, L⁴ is a covalent bond, R⁶ is methyl, and Y is a substituted or unsubstituted heteroaryl.

In another embodiment of the compounds of formula IC-3, L³ is —CH(CH₃)—, L⁴ is a covalent bond, R⁶ is methyl, and Y is a substituted or unsubstituted heteroaryl.

In another embodiment of the compounds of formula IC-3, L³ is —C(O)—, L⁴ is a covalent bond, R⁶ is methyl, and Y is a substituted or unsubstituted heteroaryl.

In another embodiment of the compounds of formula IC-3, L³ is —CH₂—, L⁴ is —C(R⁴)₂—, wherein R⁴ is as defined herein, R⁶ is methyl, and Y is a substituted or unsubstituted pyridyl.

In another embodiment of the compounds of formula IC-3, L³ is —CH(CH₃)—, L⁴ is —C(R⁴)₂— wherein R⁴ is as defined herein, R⁶ is methyl, and Y is a substituted or unsubstituted pyridyl.

In another embodiment of the compounds of formula IC-3, L³ is —C(O)—, L⁴ is —C(R⁴)₂— wherein R⁴ is as defined herein, R⁶ is methyl, and Y is a substituted or unsubstituted pyridyl.

In another embodiment of the compounds of formula IC-3, L³ is —C(R⁴)₂—, L⁴ is —CH(CH₃)— wherein R⁴ is as defined herein, R⁶ is methyl, and Y is a substituted or unsubstituted pyridyl.

In another embodiment of the compounds of formula IC-3, L³ is —CH₂—, L⁴ is —CH(CH₃)—, R⁶ is methyl, and Y is a substituted or unsubstituted pyridyl.

In another embodiment of the compounds of formula IC-3, L³ is —CH(CH₃)—, L⁴ is —CH(CH₃)—, R⁶ is methyl, and Y is a substituted or unsubstituted pyridyl.

In another embodiment of the compounds of formula IC-3, L³ is —C(O)—, L⁴ is —CH(CH₃)—, R⁶ is methyl, and Y is a substituted or unsubstituted pyridyl.

In another embodiment of the compounds of formula IC-3, L³ is —C(R⁴)₂— wherein R⁴ is as defined herein, L⁴ is a covalent bond, R⁶ is methyl, and Y is a substituted or unsubstituted pyridyl.

In another embodiment of the compounds of formula IC-3, L³ is —CH₂—, L⁴ is a covalent bond, R⁶ is methyl, and Y is a substituted or unsubstituted pyridyl.

In another embodiment of the compounds of formula IC-3, L³ is —CH(CH₃)—, L⁴ is a covalent bond, R⁶ is methyl, and Y is a substituted or unsubstituted pyridyl.

In another embodiment of the compounds of formula IC-3, L³ is —C(O)—, L⁴ is a covalent bond, R⁶ is methyl, and Y is a substituted or unsubstituted pyridyl.

In another embodiment of the compounds of formula IC-3, L³ is —CH₂—, L¹ is —C(R⁴)₂—, wherein R⁴ is as defined herein, R⁶ is methyl, and Y is a substituted or unsubstituted pyrimidyl.

In another embodiment of the compounds of formula IC-3, L³ is —CH(CH₃)—, L⁴ is —C(R⁴)₂— wherein R⁴ is as defined herein, R⁶ is methyl, and Y is a substituted or unsubstituted pyrimidyl.

In another embodiment of the compounds of formula IC-3, L³ is —C(O)—, L⁴ is —C(R⁴)₂— wherein R⁴ is as defined herein, R⁶ is methyl, and Y is a substituted or unsubstituted pyrimidyl.

In another embodiment of the compounds of formula IC-3, L³ is —C(R⁴)₂—, L⁴ is —CH(CH₃)— wherein R⁴ is as defined herein, R⁶ is methyl, and Y is a substituted or unsubstituted pyrimidyl.

In another embodiment of the compounds of formula IC-3, L³ is —CH₂—, L⁴ is —CH(CH₃)—, R⁶ is methyl, and Y is a substituted or unsubstituted pyrimidyl.

In another embodiment of the compounds of formula IC-3, L³ is —CH(CH₃)—, L⁴ is —CH(CH₃)—, R⁶ is methyl, and Y is a substituted or unsubstituted pyrimidyl.

In another embodiment of the compounds of formula IC-3, L³ is —C(O)—, L⁴ is —CH(CH₃)—, R⁶ is methyl, and Y is a substituted or unsubstituted pyrimidyl.

In another embodiment of the compounds of formula IC-3, L³ is —C(R⁴)₂— wherein R⁴ is as defined herein, L⁴ is a covalent bond, R⁶ is methyl, and Y is a substituted or unsubstituted pyrimidyl.

In another embodiment of the compounds of formula IC-3, L³ is —CH₂—, L⁴ is a covalent bond, R⁶ is methyl, and Y is a substituted or unsubstituted pyrimidyl.

In another embodiment of the compounds of formula IC-3, L³ is —CH(CH₃)—, L⁴ is a covalent bond, R⁶ is methyl, and Y is a substituted or unsubstituted pyrimidyl.

In another embodiment of the compounds of formula IC-3, L³ is —C(O)—, L⁴ is a covalent bond, R⁶ is methyl, and Y is a substituted or unsubstituted pyrimidyl.

In another embodiment of the compounds of formula IC-3, L³ is —CH₂—, L⁴ is —C(R⁴)₂—, wherein R⁴ is as defined herein, R⁶ is methyl, and Y is a substituted or unsubstituted quinolyl.

In another embodiment of the compounds of formula IC-3, L³ is —CH(CH₃)—, L⁴ is —C(R⁴)₂— wherein R⁴ is as defined herein, R⁶ is methyl, and Y is a substituted or unsubstituted quinolyl.

In another embodiment of the compounds of formula IC-3, L³ is —C(O)—, L⁴ is —C(R⁴)₂— wherein R⁴ is as defined herein, R⁶ is methyl, and Y is a substituted or unsubstituted quinolyl.

In another embodiment of the compounds of formula IC-3, L³ is —C(R⁴)₂—, L⁴ is —CH(CH₃)— wherein R⁴ is as defined herein, R⁶ is methyl, and Y is a substituted or unsubstituted quinolyl.

In another embodiment of the compounds of formula IC-3, L³ is —CH₂—, L⁴ is —CH(CH₃)—, R⁶ is methyl, and Y is a substituted or unsubstituted quinolyl.

In another embodiment of the compounds of formula IC-3, L³ is —CH(CH₃)—, L⁴ is —CH(CH₃)—, R⁶ is methyl, and Y is a substituted or unsubstituted quinolyl.

In another embodiment of the compounds of formula IC-3, L³ is —C(O)—, L⁴ is —CH(CH₃)—, R⁶ is methyl, and Y is a substituted or unsubstituted quinolyl.

In another embodiment of the compounds of formula IC-3, L³ is —C(R⁴)₂— wherein R⁴ is as defined herein, L⁴ is a covalent bond, R⁶ is methyl, and Y is a substituted or unsubstituted quinolyl.

In another embodiment of the compounds of formula IC-3, L³ is —CH₂—, L⁴ is a covalent bond, R⁶ is methyl, and Y is a substituted or unsubstituted quinolyl.

In another embodiment of the compounds of formula IC-3, L³ is —CH(CH₃)—, L⁴ is a covalent bond, R⁶ is methyl, and Y is a substituted or unsubstituted quinolyl.

In another embodiment of the compounds of formula IC-3, L³ is —C(O)—, L⁴ is a covalent bond, R⁶ is methyl, and Y is a substituted or unsubstituted quinolyl.

In another embodiment of the compounds of formula IC-3, L³ is —CH₂—, L⁴ is —C(R⁴)₂—, wherein R⁴ is as defined herein, R⁶ is methyl, and Y is a substituted or unsubstituted indolyl.

In another embodiment of the compounds of formula IC-3, L³ is —CH(CH₃)—, L⁴ is —C(R⁴)₂— wherein R⁴ is as defined herein, R⁶ is methyl, and Y is a substituted or unsubstituted indolyl.

In another embodiment of the compounds of formula IC-3, L³ is —C(O)—, L⁴ is —C(R⁴)₂— wherein R⁴ is as defined herein, R⁶ is methyl, and Y is a substituted or unsubstituted indolyl.

In another embodiment of the compounds of formula IC-3, L³ is —C(R⁴)₂—, L⁴ is —CH(CH₃)— wherein R⁴ is as defined herein, R⁶ is methyl, and Y is a substituted or unsubstituted indolyl.

In another embodiment of the compounds of formula IC-3, L³ is —CH₂—, L⁴ is —CH(CH₃)—, R⁶ is methyl, and Y is a substituted or unsubstituted indolyl.

In another embodiment of the compounds of formula IC-3, L³ is —CH(CH₃)—, L⁴ is —CH(CH₃)—, R⁶ is methyl, and Y is a substituted or unsubstituted indolyl.

In another embodiment of the compounds of formula IC-3, L³ is —C(O)—, L⁴ is —CH(CH₃)—, R⁶ is methyl, and Y is a substituted or unsubstituted indolyl.

In another embodiment of the compounds of formula IC-3, L³ is —C(R⁴)₂— wherein R⁴ is as defined herein, L⁴ is a covalent bond, R⁶ is methyl, and Y is a substituted or unsubstituted indolyl.

In another embodiment of the compounds of formula IC-3, L³ is —CH₂—, L⁴ is a covalent bond, R⁶ is methyl, and Y is a substituted or unsubstituted indolyl.

In another embodiment of the compounds of formula IC-3, L³ is —CH(CH₃)—, L⁴ is a covalent bond, R⁶ is methyl, and Y is a substituted or unsubstituted indolyl.

In another embodiment of the compounds of formula IC-3, L³ is —C(O)—, L⁴ is a covalent bond, R⁶ is methyl, and Y is a substituted or unsubstituted indolyl.

In another embodiment of the compounds of formula IC-3, L³ is —CH₂—, L⁴ is —C(R⁴)₂—, wherein R⁴ is as defined herein, R⁶ is methyl, and Y is a substituted or unsubstituted thienyl.

In another embodiment of the compounds of formula IC-3, L³ is —CH(CH₃)—, L⁴ is —C(R⁴)₂— wherein R⁴ is as defined herein, R⁶ is methyl, and Y is a substituted or unsubstituted thienyl.

In another embodiment of the compounds of formula IC-3, L³ is —C(O)—, L⁴ is —C(R⁴)₂— wherein R⁴ is as defined herein, R⁶ is methyl, and Y is a substituted or unsubstituted thienyl.

In another embodiment of the compounds of formula IC-3, L³ is —C(R⁴)₂—, L⁴ is —CH(CH₃)— wherein R⁴ is as defined herein, R⁶ is methyl, and Y is a substituted or unsubstituted thienyl.

In another embodiment of the compounds of formula IC-3, L³ is —CH₂—, L⁴ is —CH(CH₃)—, R⁶ is methyl, and Y is a substituted or unsubstituted thienyl.

In another embodiment of the compounds of formula IC-3, L³ is —CH(CH₃)—, L⁴ is —CH(CH₃)—, R⁶ is methyl, and Y is a substituted or unsubstituted thienyl.

In another embodiment of the compounds of formula IC-3, L³ is —C(O)—, L⁴ is —CH(CH₃)—, R⁶ is methyl, and Y is a substituted or unsubstituted thienyl.

In another embodiment of the compounds of formula IC-3, L³ is —C(R⁴)₂— wherein R⁴ is as defined herein, L⁴ is a covalent bond, R⁶ is methyl, and Y is a substituted or unsubstituted thienyl.

In another embodiment of the compounds of formula IC-3, L³ is —CH₂—, L⁴ is a covalent bond, R⁶ is methyl, and Y is a substituted or unsubstituted thienyl.

In another embodiment of the compounds of formula IC-3, L³ is —CH(CH₃)—, L⁴ is a covalent bond, R⁶ is methyl, and Y is a substituted or unsubstituted thienyl.

In another embodiment of the compounds of formula IC-3, L³ is —C(O)—, L⁴ is a covalent bond, R⁶ is methyl, and Y is a substituted or unsubstituted thienyl.

In another embodiment of the compounds of formula IC-3, L³ is —CH₂—, L⁴ is —C(R⁴)₂—, wherein R⁴ is as defined herein, R⁶ is methyl, and Y is a substituted or unsubstituted purinyl.

In another embodiment of the compounds of formula IC-3, L³ is —CH(CH₃)—, L⁴ is —C(R⁴)₂— wherein R⁴ is as defined herein, R⁶ is methyl, and Y is a substituted or unsubstituted purinyl.

In another embodiment of the compounds of formula IC-3, L³ is —C(O)—, L⁴ is —C(R⁴)₂— wherein R⁴ is as defined herein, R⁶ is methyl, and Y is a substituted or unsubstituted purinyl.

In another embodiment of the compounds of formula IC-3, L³ is —C(R⁴)₂—, L⁴ is —CH(CH₃)— wherein R⁴ is as defined herein, R⁶ is methyl, and Y is a substituted or unsubstituted purinyl.

In another embodiment of the compounds of formula IC-3, L³ is —CH₂—, L⁴ is —CH(CH₃)—, R⁶ is methyl, and Y is a substituted or unsubstituted purinyl.

In another embodiment of the compounds of formula IC-3, L³ is —CH(CH₃)—, L⁴ is —CH(CH₃)—, R⁶ is methyl, and Y is a substituted or unsubstituted purinyl.

In another embodiment of the compounds of formula IC-3, L³ is —C(O)—, L⁴ is —CH(CH₃)—, R⁶ is methyl, and Y is a substituted or unsubstituted purinyl.

In another embodiment of the compounds of formula IC-3, L³ is —C(R⁴)₂— wherein R⁴ is as defined herein, L⁴ is a covalent bond, R⁶ is methyl, and Y is a substituted or unsubstituted purinyl.

In another embodiment of the compounds of formula IC-3, L³ is —CH₂—, L⁴ is a covalent bond, R⁶ is methyl, and Y is a substituted or unsubstituted purinyl.

In another embodiment of the compounds of formula IC-3, L³ is —CH(CH₃)—, L⁴ is a covalent bond, R⁶ is methyl, and Y is a substituted or unsubstituted purinyl.

In another embodiment of the compounds of formula IC-3, L³ is —C(O)—, L⁴ is a covalent bond, R⁶ is methyl, and Y is a substituted or unsubstituted purinyl.

In another embodiment of the compounds of formula IC-3, L³ is —CH₂—, L⁴ is —C(R⁴)₂—, wherein R⁴ is as defined herein, R⁶ is methyl, and Y is a substituted or unsubstituted furanyl.

In another embodiment of the compounds of formula IC-3, L³ is —CH(CH₃)—, L⁴ is —C(R⁴)₂— wherein R⁴ is as defined herein, R⁶ is methyl, and Y is a substituted or unsubstituted furanyl.

In another embodiment of the compounds of formula IC-3, L³ is —C(O)—, L⁴ is —C(R⁴)₂— wherein R⁴ is as defined herein, R⁶ is methyl, and Y is a substituted or unsubstituted furanyl.

In another embodiment of the compounds of formula IC-3, L³ is —C(R⁴)₂—, L⁴ is —CH(CH₃)— wherein R⁴ is as defined herein, R⁶ is methyl, and Y is a substituted or unsubstituted furanyl.

In another embodiment of the compounds of formula IC-3, L³ is —CH₂—, L⁴ is —CH(CH₃)—, R⁶ is methyl, and Y is a substituted or unsubstituted furanyl.

In another embodiment of the compounds of formula IC-3, L³ is —CH(CH₃)—, L⁴ is —CH(CH₃)—, R⁶ is methyl, and Y is a substituted or unsubstituted furanyl.

In another embodiment of the compounds of formula IC-3, L³ is —C(O)—, L⁴ is —CH(CH₃)—, R⁶ is methyl, and Y is a substituted or unsubstituted furanyl.

In another embodiment of the compounds of formula IC-3, L³ is —C(R⁴)₂— wherein R⁴ is as defined herein, L⁴ is a covalent bond, R⁶ is methyl, and Y is a substituted or unsubstituted furanyl.

In another embodiment of the compounds of formula IC-3, L³ is —CH₂—, L⁴ is a covalent bond, R⁶ is methyl, and Y is a substituted or unsubstituted furanyl.

In another embodiment of the compounds of formula IC-3, L³ is —CH(CH₃)—, L⁴ is a covalent bond, R⁶ is methyl, and Y is a substituted or unsubstituted furanyl.

In another embodiment of the compounds of formula IC-3, L³ is —C(O)—, L⁴ is a covalent bond, R⁶ is methyl, and Y is a substituted or unsubstituted furanyl.

In another embodiment of the compounds of formula IC-3, L³ is —CH₂—, L⁴ is —C(R⁴)₂—, wherein R⁴ is as defined herein, R⁶ is methyl, and Y is a substituted or unsubstituted pyrrolyl.

In another embodiment of the compounds of formula IC-3, L³ is —CH(CH₃)—, L⁴ is —C(R⁴)₂— wherein R⁴ is as defined herein, R⁶ is methyl, and Y is a substituted or unsubstituted pyrrolyl.

In another embodiment of the compounds of formula IC-3, L³ is —C(O)—, L⁴ is —C(R⁴)₂— wherein R⁴ is as defined herein, R⁶ is methyl, and Y is a substituted or unsubstituted pyrrolyl.

In another embodiment of the compounds of formula IC-3, L³ is —C(R⁴)₂—, L⁴ is —CH(CH₃)— wherein R⁴ is as defined herein, R⁶ is methyl, and Y is a substituted or unsubstituted pyrrolyl.

In another embodiment of the compounds of formula IC-3, L³ is —CH₂—, L⁴ is —CH(CH₃)—, R⁶ is methyl, and Y is a substituted or unsubstituted pyrrolyl.

In another embodiment of the compounds of formula IC-3, L³ is —CH(CH₃)—, L⁴ is —CH(CH₃)—, R⁶ is methyl, and Y is a substituted or unsubstituted pyrrolyl.

In another embodiment of the compounds of formula IC-3, L³ is —C(O)—, L⁴ is —CH(CH₃)—, R⁶ is methyl, and Y is a substituted or unsubstituted pyrrolyl.

In another embodiment of the compounds of formula IC-3, L³ is —C(R⁴)₂— wherein R⁴ is as defined herein, L⁴ is a covalent bond, R⁶ is methyl, and Y is a substituted or unsubstituted pyrrolyl.

In another embodiment of the compounds of formula IC-3, L³ is —CH₂—, L⁴ is a covalent bond, R⁶ is methyl, and Y is a substituted or unsubstituted pyrrolyl.

In another embodiment of the compounds of formula IC-3, L³ is —CH(CH₃)—, L⁴ is a covalent bond, R⁶ is methyl, and Y is a substituted or unsubstituted pyrrolyl.

In another embodiment of the compounds of formula IC-3, L³ is —C(O)—, L⁴ is a covalent bond, R⁶ is methyl, and Y is a substituted or unsubstituted pyrrolyl.

In another embodiment of the compounds of formula IC-3, L³ is —CH₂—, L⁴ is —C(R⁴)₂—, wherein R⁴ is as defined herein, R⁶ is methyl, and Y is alkoxyalkyl.

In another embodiment of the compounds of formula IC-3, L³ is —CH(CH₃)—, L⁴ is —C(R⁴)₂— wherein R⁴ is as defined herein, R⁶ is methyl, and Y is alkoxyalkyl.

In another embodiment of the compounds of formula IC-3, L³ is —C(O)—, L⁴ is —C(R⁴)₂— wherein R⁴ is as defined herein, R⁶ is methyl, and Y is alkoxyalkyl.

In another embodiment of the compounds of formula IC-3, L³ is —C(R⁴)₂—, L⁴ is —CH(CH₃)— wherein R⁴ is as defined herein, R⁶ is methyl, and Y is alkoxyalkyl.

In another embodiment of the compounds of formula IC-3, L³ is —CH₂—, L⁴ is —CH(CH₃)—, R⁶ is methyl, and Y is alkoxyalkyl.

In another embodiment of the compounds of formula IC-3, L³ is —CH(CH₃)—, L⁴ is —CH(CH₃)—, R⁶ is methyl, and Y is alkoxyalkyl.

In another embodiment of the compounds of formula IC-3, L³ is —C(O)—, L⁴ is —CH(CH₃)—, R⁶ is methyl, and Y is alkoxyalkyl.

In another embodiment of the compounds of formula IC-3, L³ is —C(R⁴)₂— wherein R⁴ is as defined herein, L⁴ is a covalent bond, R⁶ is methyl, and Y is alkoxyalkyl.

In another embodiment of the compounds of formula IC-3, L³ is —CH₂—, L⁴ is a covalent bond, R⁶ is methyl, and Y is alkoxyalkyl.

In another embodiment of the compounds of formula IC-3, L³ is —CH(CH₃)—, L⁴ is a covalent bond, R⁶ is methyl, and Y is alkoxyalkyl.

In another embodiment of the compounds of formula IC-3, L³ is —C(O)—, L⁴ is a covalent bond, R⁶ is methyl, and Y is alkoxyalkyl.

In another embodiment of the compounds of formula IC-3, L³ is —C(R⁴)₂—, L⁴ is —C(R⁴)₂—, wherein R⁴ is as defined herein, R⁶ is alkoxyalkyl, and Y is a substituted or unsubstituted aryl.

In another embodiment of the compounds of formula IC-3, L³ is —CH₂—, L⁴ is —C(R⁴)₂—, wherein R⁴ is as defined herein, R⁶ is alkoxyalkyl, and Y is a substituted or unsubstituted aryl.

In another embodiment of the compounds of formula IC-3, L³ is —CH(CH₃)—, L⁴ is —C(R⁴)₂— wherein R⁴ is as defined herein, R⁶ is alkoxyalkyl, and Y is a substituted or unsubstituted aryl.

In another embodiment of the compounds of formula IC-3, L³ is —C(O)—, L⁴ is —C(R⁴)₂— wherein R⁴ is as defined herein, R⁶ is alkoxyalkyl, and Y is a substituted or unsubstituted aryl.

In another embodiment of the compounds of formula IC-3, L³ is —C(R⁴)₂—, L⁴ is —CH(CH₃)— wherein R⁴ is as defined herein, R⁶ is alkoxyalkyl, and Y is a substituted or unsubstituted aryl.

In another embodiment of the compounds of formula IC-3, L³ is —CH₂—, L⁴ is —CH(CH₃)—, R⁶ is alkoxyalkyl, and Y is a substituted or unsubstituted aryl.

In another embodiment of the compounds of formula IC-3, L³ is —CH(CH₃)—, L⁴ is —CH(CH₃)—, R⁶ is alkoxyalkyl, and Y is a substituted or unsubstituted aryl.

In another embodiment of the compounds of formula IC-3, L³ is —C(O)—, L⁴ is —CH(CH₃)—, R⁶ is alkoxyalkyl, and Y is a substituted or unsubstituted aryl.

In another embodiment of the compounds of formula IC-3, L³ is —C(R⁴)₂— wherein R⁴ is as defined herein, L⁴ is a covalent bond, R⁶ is alkoxyalkyl, and Y is a substituted or unsubstituted aryl.

In another embodiment of the compounds of formula IC-3, L³ is —CH₂—, L⁴ is a covalent bond, R⁶ is alkoxyalkyl, and Y is a substituted or unsubstituted aryl.

In another embodiment of the compounds of formula IC-3, L³ is —CH(CH₃)—, L⁴ is a covalent bond, R⁶ is alkoxyalkyl, and Y is a substituted or unsubstituted aryl.

In another embodiment of the compounds of formula IC-3, L³ is —C(O)—, L⁴ is a covalent bond, R⁶ is alkoxyalkyl, and Y is a substituted or unsubstituted aryl.

In another embodiment of the compounds of formula IC-3, L³ is —C(R⁴)₂—, L⁴ is —C(R⁴)₂—, wherein R⁴ is as defined herein, R⁶ is alkoxyalkyl, and Y is a substituted or unsubstituted phenyl.

In another embodiment of the compounds of formula IC-3, L³ is —CH₂—, L⁴ is —C(R⁴)₂—, wherein R⁴ is as defined herein, R⁶ is alkoxyalkyl, and Y is a substituted or unsubstituted phenyl.

In another embodiment of the compounds of formula IC-3, L³ is —CH(CH₃)—, L⁴ is —C(R⁴)₂— wherein R⁴ is as defined herein, R⁶ is alkoxyalkyl, and Y is a substituted or unsubstituted phenyl.

In another embodiment of the compounds of formula IC-3, L³ is —C(O)—, L⁴ is —C(R⁴)₂— wherein R⁴ is as defined herein, R⁶ is alkoxyalkyl, and Y is a substituted or unsubstituted phenyl.

In another embodiment of the compounds of formula IC-3, L³ is —C(R⁴)₂—, L⁴ is —CH(CH₃)— wherein R⁴ is as defined herein, R⁶ is alkoxyalkyl, and Y is a substituted or unsubstituted phenyl.

In another embodiment of the compounds of formula IC-3, L³ is —CH₂—, L⁴ is —CH(CH₃)—, R⁶ is alkoxyalkyl, and Y is a substituted or unsubstituted phenyl.

In another embodiment of the compounds of formula IC-3, L³ is —CH(CH₃)—, L⁴ is —CH(CH₃)—, R⁶ is alkoxyalkyl, and Y is a substituted or unsubstituted phenyl.

In another embodiment of the compounds of formula IC-3, L³ is —C(O)—, L⁴ is —CH(CH₃)—, R⁶ is alkoxyalkyl, and Y is a substituted or unsubstituted phenyl.

In another embodiment of the compounds of formula IC-3, L³ is —C(R⁴)₂— wherein R⁴ is as defined herein, L⁴ is a covalent bond, R⁶ is alkoxyalkyl, and Y is a substituted or unsubstituted phenyl.

In another embodiment of the compounds of formula IC-3, L³ is —CH₂—, L⁴ is a covalent bond, R⁶ is alkoxyalkyl, and Y is a substituted or unsubstituted phenyl.

In another embodiment of the compounds of formula IC-3, L³ is —CH(CH₃)—, L⁴ is a covalent bond, R⁶ is alkoxyalkyl, and Y is a substituted or unsubstituted phenyl.

In another embodiment of the compounds of formula IC-3, L³ is —C(O)—, L⁴ is a covalent bond, R⁶ is alkoxyalkyl, and Y is a substituted or unsubstituted phenyl.

In another embodiment of the compounds of formula IC-3, L³ is —C(R⁴)₂—, L⁴ is —C(R⁴)₂—, wherein R⁴ is as defined herein, R⁶ is alkoxyalkyl, and Y is a substituted or unsubstituted heteroaryl.

In another embodiment of the compounds of formula IC-3, L³ is —CH₂—, L⁴ is —C(R⁴)₂—, wherein R⁴ is as defined herein, R⁶ is alkoxyalkyl, and Y is a substituted or unsubstituted heteroaryl.

In another embodiment of the compounds of formula IC-3, L³ is —CH(CH₃)—, L⁴ is —C(R⁴)₂— wherein R⁴ is as defined herein, R⁶ is alkoxyalkyl, and Y is a substituted or unsubstituted heteroaryl.

In another embodiment of the compounds of formula IC-3, L³ is —C(O)—, L⁴ is —C(R⁴)₂— wherein R⁴ is as defined herein, R⁶ is alkoxyalkyl, and Y is a substituted or unsubstituted heteroaryl.

In another embodiment of the compounds of formula IC-3, L³ is —C(R⁴)₂—, L⁴ is —CH(CH₃)— wherein R⁴ is as defined herein, R⁶ is alkoxyalkyl, and Y is a substituted or unsubstituted heteroaryl.

In another embodiment of the compounds of formula IC-3, L³ is —CH₂—, L⁴ is —CH(CH₃)—, R⁶ is alkoxyalkyl, and Y is a substituted or unsubstituted heteroaryl.

In another embodiment of the compounds of formula IC-3, L³ is —CH(CH₃)—, L⁴ is —CH(CH₃)—, R⁶ is alkoxyalkyl, and Y is a substituted or unsubstituted heteroaryl.

In another embodiment of the compounds of formula IC-3, L³ is —C(O)—, L⁴ is —CH(CH₃)—, R⁶ is alkoxyalkyl, and Y is a substituted or unsubstituted heteroaryl.

In another embodiment of the compounds of formula IC-3, L³ is —C(R⁴)₂— wherein R⁴ is as defined herein, L⁴ is a covalent bond, R⁶ is alkoxyalkyl, and Y is a substituted or unsubstituted heteroaryl.

In another embodiment of the compounds of formula IC-3, L³ is —CH₂—, L⁴ is a covalent bond, R⁶ is alkoxyalkyl, and Y is a substituted or unsubstituted heteroaryl.

In another embodiment of the compounds of formula IC-3, L³ is —CH(CH₃)—, L⁴ is a covalent bond, R⁶ is alkoxyalkyl, and Y is a substituted or unsubstituted heteroaryl.

In another embodiment of the compounds of formula IC-3, L³ is —C(O)—, L⁴ is a covalent bond, R⁶ is alkoxyalkyl, and Y is a substituted or unsubstituted heteroaryl.

In another embodiment of the compounds of formula IC-3, L³ is —CH₂—, L⁴ is —C(R⁴)₂—, wherein R⁴ is as defined herein, R⁶ is alkoxyalkyl, and Y is a substituted or unsubstituted pyridyl.

In another embodiment of the compounds of formula IC-3, L³ is —CH(CH₃)—, L⁴ is —C(R⁴)₂— wherein R⁴ is as defined herein, R⁶ is alkoxyalkyl, and Y is a substituted or unsubstituted pyridyl.

In another embodiment of the compounds of formula IC-3, L³ is —C(O)—, L⁴ is —C(R⁴)₂— wherein R⁴ is as defined herein, R⁶ is alkoxyalkyl, and Y is a substituted or unsubstituted pyridyl.

In another embodiment of the compounds of formula IC-3, L³ is —C(R⁴)₂—, L⁴ is —CH(CH₃)— wherein R⁴ is as defined herein, R⁶ is alkoxyalkyl, and Y is a substituted or unsubstituted pyridyl.

In another embodiment of the compounds of formula IC-3, L³ is —CH₂—, L⁴ is —CH(CH₃)—, R⁶ is alkoxyalkyl, and Y is a substituted or unsubstituted pyridyl.

In another embodiment of the compounds of formula IC-3, L³ is —CH(CH₃)—, L⁴ is —CH(CH₃)—, R⁶ is alkoxyalkyl, and Y is a substituted or unsubstituted pyridyl.

In another embodiment of the compounds of formula IC-3, L³ is —C(O)—, L⁴ is —CH(CH₃)—, R⁶ is alkoxyalkyl, and Y is a substituted or unsubstituted pyridyl.

In another embodiment of the compounds of formula IC-3, L³ is —C(R⁴)₂— wherein R⁴ is as defined herein, L⁴ is a covalent bond, R⁶ is alkoxyalkyl, and Y is a substituted or unsubstituted pyridyl.

In another embodiment of the compounds of formula IC-3, L³ is —CH₂—, L⁴ is a covalent bond, R⁶ is alkoxyalkyl, and Y is a substituted or unsubstituted pyridyl.

In another embodiment of the compounds of formula IC-3, L³ is —CH(CH₃)—, L⁴ is a covalent bond, R⁶ is alkoxyalkyl, and Y is a substituted or unsubstituted pyridyl.

In another embodiment of the compounds of formula IC-3, L³ is —C(O)—, L⁴ is a covalent bond, R⁶ is alkoxyalkyl, and Y is a substituted or unsubstituted pyridyl.

In another embodiment of the compounds of formula IC-3, L³ is —CH₂—, L⁴ is —C(R⁴)₂—, wherein R⁴ is as defined herein, R⁶ is alkoxyalkyl, and Y is a substituted or unsubstituted pyrimidyl.

In another embodiment of the compounds of formula IC-3, L³ is —CH(CH₃)—, L⁴ is —C(R⁴)₂— wherein R⁴ is as defined herein, R⁶ is alkoxyalkyl, and Y is a substituted or unsubstituted pyrimidyl.

In another embodiment of the compounds of formula IC-3, L³ is —C(O)—, L⁴ is —C(R⁴)₂— wherein R⁴ is as defined herein, R⁶ is alkoxyalkyl, and Y is a substituted or unsubstituted pyrimidyl.

In another embodiment of the compounds of formula IC-3, L³ is —C(R⁴)₂—, L⁴ is —CH(CH₃)— wherein R⁴ is as defined herein, R⁶ is alkoxyalkyl, and Y is a substituted or unsubstituted pyrimidyl.

In another embodiment of the compounds of formula IC-3, L³ is —CH₂—, L⁴ is —CH(CH₃)—, R⁶ is alkoxyalkyl, and Y is a substituted or unsubstituted pyrimidyl.

In another embodiment of the compounds of formula IC-3, L³ is —CH(CH₃)—, L⁴ is —CH(CH₃)—, R⁶ is alkoxyalkyl, and Y is a substituted or unsubstituted pyrimidyl.

In another embodiment of the compounds of formula IC-3, L³ is —C(O)—, L⁴ is —CH(CH₃)—, R⁶ is alkoxyalkyl, and Y is a substituted or unsubstituted pyrimidyl.

In another embodiment of the compounds of formula IC-3, L³ is —C(R⁴)₂— wherein R⁴ is as defined herein, L⁴ is a covalent bond, R⁶ is alkoxyalkyl, and Y is a substituted or unsubstituted pyrimidyl.

In another embodiment of the compounds of formula IC-3, L³ is —CH₂—, L⁴ is a covalent bond, R⁶ is alkoxyalkyl, and Y is a substituted or unsubstituted pyrimidyl.

In another embodiment of the compounds of formula IC-3, L³ is —CH(CH₃)—, L⁴ is a covalent bond, R⁶ is alkoxyalkyl, and Y is a substituted or unsubstituted pyrimidyl.

In another embodiment of the compounds of formula IC-3, L³ is —C(O)—, L⁴ is a covalent bond, R⁶ is alkoxyalkyl, and Y is a substituted or unsubstituted pyrimidyl.

In another embodiment of the compounds of formula IC-3, L³ is —CH₂—, L⁴ is —C(R⁴)₂—, wherein R⁴ is as defined herein, R⁶ is alkoxyalkyl, and Y is a substituted or unsubstituted quinolyl.

In another embodiment of the compounds of formula IC-3, L³ is —CH(CH₃)—, L⁴ is —C(R⁴)₂— wherein R⁴ is as defined herein, R⁶ is alkoxyalkyl, and Y is a substituted or unsubstituted quinolyl.

In another embodiment of the compounds of formula IC-3, L³ is —C(O)—, L⁴ is —C(R⁴)₂— wherein R⁴ is as defined herein, R⁶ is alkoxyalkyl, and Y is a substituted or unsubstituted quinolyl.

In another embodiment of the compounds of formula IC-3, L³ is —C(R⁴)₂—, L⁴ is —CH(CH₃)— wherein R⁴ is as defined herein, R⁶ is alkoxyalkyl, and Y is a substituted or unsubstituted quinolyl.

In another embodiment of the compounds of formula IC-3, L³ is —CH₂—, L⁴ is —CH(CH₃)—, R⁶ is alkoxyalkyl, and Y is a substituted or unsubstituted quinolyl.

In another embodiment of the compounds of formula IC-3, L³ is —CH(CH₃)—, L⁴ is —CH(CH₃)—, R⁶ is alkoxyalkyl, and Y is a substituted or unsubstituted quinolyl.

In another embodiment of the compounds of formula IC-3, L³ is —C(O)—, L⁴ is —CH(CH₃)—, R⁶ is alkoxyalkyl, and Y is a substituted or unsubstituted quinolyl.

In another embodiment of the compounds of formula IC-3, L³ is —C(R⁴)₂— wherein R⁴ is as defined herein, L⁴ is a covalent bond, R⁶ is alkoxyalkyl, and Y is a substituted or unsubstituted quinolyl.

In another embodiment of the compounds of formula IC-3, L³ is —CH₂—, L⁴ is a covalent bond, R⁶ is alkoxyalkyl, and Y is a substituted or unsubstituted quinolyl.

In another embodiment of the compounds of formula IC-3, L³ is —CH(CH₃)—, L⁴ is a covalent bond, R⁶ is alkoxyalkyl, and Y is a substituted or unsubstituted quinolyl.

In another embodiment of the compounds of formula IC-3, L³ is —C(O)—, L⁴ is a covalent bond, R⁶ is alkoxyalkyl, and Y is a substituted or unsubstituted quinolyl.

In another embodiment of the compounds of formula IC-3, L³ is —CH₂—, L⁴ is —C(R⁴)₂—, wherein R⁴ is as defined herein, R⁶ is alkoxyalkyl, and Y is a substituted or unsubstituted indolyl.

In another embodiment of the compounds of formula IC-3, L³ is —CH(CH₃)—, L⁴ is —C(R⁴)₂— wherein R⁴ is as defined herein, R⁶ is alkoxyalkyl, and Y is a substituted or unsubstituted indolyl.

In another embodiment of the compounds of formula IC-3, L³ is —C(O)—, L⁴ is —C(R⁴)₂— wherein R⁴ is as defined herein, R⁶ is alkoxyalkyl, and Y is a substituted or unsubstituted indolyl.

In another embodiment of the compounds of formula IC-3, L³ is —C(R⁴)₂—, L⁴ is —CH(CH₃)— wherein R⁴ is as defined herein, R⁶ is alkoxyalkyl, and Y is a substituted or unsubstituted indolyl.

In another embodiment of the compounds of formula IC-3, L³ is —CH₂—, L⁴ is —CH(CH₃)—, R⁶ is alkoxyalkyl, and Y is a substituted or unsubstituted indolyl.

In another embodiment of the compounds of formula IC-3, L³ is —CH(CH₃)—, L⁴ is —CH(CH₃)—, R⁶ is alkoxyalkyl, and Y is a substituted or unsubstituted indolyl.

In another embodiment of the compounds of formula IC-3, L³ is —C(O)—, L⁴ is —CH(CH₃)—, R⁶ is alkoxyalkyl, and Y is a substituted or unsubstituted indolyl.

In another embodiment of the compounds of formula IC-3, L³ is —C(R⁴)₂— wherein R⁴ is as defined herein, L⁴ is a covalent bond, R⁶ is alkoxyalkyl, and Y is a substituted or unsubstituted indolyl.

In another embodiment of the compounds of formula IC-3, L³ is —CH₂—, L⁴ is a covalent bond, R⁶ is alkoxyalkyl, and Y is a substituted or unsubstituted indolyl.

In another embodiment of the compounds of formula IC-3, L³ is —CH(CH₃)—, L⁴ is a covalent bond, R⁶ is alkoxyalkyl, and Y is a substituted or unsubstituted indolyl.

In another embodiment of the compounds of formula IC-3, L³ is —C(O)—, L⁴ is a covalent bond, R⁶ is alkoxyalkyl, and Y is a substituted or unsubstituted indolyl.

In another embodiment of the compounds of formula IC-3, L³ is —CH₂—, L⁴ is —C(R⁴)₂—, wherein R⁴ is as defined herein, R⁶ is alkoxyalkyl, and Y is a substituted or unsubstituted thienyl.

In another embodiment of the compounds of formula IC-3, L³ is —CH(CH₃)—, L⁴ is —C(R⁴)₂— wherein R⁴ is as defined herein, R⁶ is alkoxyalkyl, and Y is a substituted or unsubstituted thienyl.

In another embodiment of the compounds of formula IC-3, L³ is —C(O)—, L⁴ is —C(R⁴)₂— wherein R⁴ is as defined herein, R⁶ is alkoxyalkyl, and Y is a substituted or unsubstituted thienyl.

In another embodiment of the compounds of formula IC-3, L³ is —C(R⁴)₂—, L⁴ is —CH(CH₃)— wherein R⁴ is as defined herein, R⁶ is alkoxyalkyl, and Y is a substituted or unsubstituted thienyl.

In another embodiment of the compounds of formula IC-3, L³ is —CH₂—, L⁴ is —CH(CH₃)—, R⁶ is alkoxyalkyl, and Y is a substituted or unsubstituted thienyl.

In another embodiment of the compounds of formula IC-3, L³ is —CH(CH₃)—, L⁴ is —CH(CH₃)—, R⁶ is alkoxyalkyl, and Y is a substituted or unsubstituted thienyl.

In another embodiment of the compounds of formula IC-3, L³ is —C(O)—, L⁴ is —CH(CH₃)—, R⁶ is alkoxyalkyl, and Y is a substituted or unsubstituted thienyl.

In another embodiment of the compounds of formula IC-3, L³ is —C(R⁴)₂— wherein R⁴ is as defined herein, L⁴ is a covalent bond, R⁶ is alkoxyalkyl, and Y is a substituted or unsubstituted thienyl.

In another embodiment of the compounds of formula IC-3, L³ is —CH₂—, L⁴ is a covalent bond, R⁶ is alkoxyalkyl, and Y is a substituted or unsubstituted thienyl.

In another embodiment of the compounds of formula IC-3, L³ is —CH(CH₃)—, L⁴ is a covalent bond, R⁶ is alkoxyalkyl, and Y is a substituted or unsubstituted thienyl.

In another embodiment of the compounds of formula IC-3, L³ is —C(O)—, L⁴ is a covalent bond, R⁶ is alkoxyalkyl, and Y is a substituted or unsubstituted thienyl.

In another embodiment of the compounds of formula IC-3, L³ is —CH₂—, L⁴ is —C(R⁴)₂—, wherein R⁴ is as defined herein, R⁶ is alkoxyalkyl, and Y is a substituted or unsubstituted purinyl.

In another embodiment of the compounds of formula IC-3, L³ is —CH(CH₃)—, L⁴ is —C(R⁴)₂— wherein R⁴ is as defined herein, R⁶ is alkoxyalkyl, and Y is a substituted or unsubstituted purinyl.

In another embodiment of the compounds of formula IC-3, L³ is —C(O)—, L⁴ is —C(R⁴)₂— wherein R⁴ is as defined herein, R⁶ is alkoxyalkyl, and Y is a substituted or unsubstituted purinyl.

In another embodiment of the compounds of formula IC-3, L³ is —C(R⁴)₂—, L⁴ is —CH(CH₃)— wherein R⁴ is as defined herein, R⁶ is alkoxyalkyl, and Y is a substituted or unsubstituted purinyl.

In another embodiment of the compounds of formula IC-3, L³ is —CH₂—, L⁴ is —CH(CH₃)—, R⁶ is alkoxyalkyl, and Y is a substituted or unsubstituted purinyl.

In another embodiment of the compounds of formula IC-3, L³ is —CH(CH₃)—, L⁴ is —CH(CH₃)—, R⁶ is alkoxyalkyl, and Y is a substituted or unsubstituted purinyl.

In another embodiment of the compounds of formula IC-3, L³ is —C(O)—, L⁴ is —CH(CH₃)—, R⁶ is alkoxyalkyl, and Y is a substituted or unsubstituted purinyl.

In another embodiment of the compounds of formula IC-3, L³ is —C(R⁴)₂— wherein R⁴ is as defined herein, L⁴ is a covalent bond, R⁶ is alkoxyalkyl, and Y is a substituted or unsubstituted purinyl.

In another embodiment of the compounds of formula IC-3, L³ is —CH₂—, L⁴ is a covalent bond, R⁶ is alkoxyalkyl, and Y is a substituted or unsubstituted purinyl.

In another embodiment of the compounds of formula IC-3, L³ is —CH(CH₃)—, L⁴ is a covalent bond, R⁶ is alkoxyalkyl, and Y is a substituted or unsubstituted purinyl.

In another embodiment of the compounds of formula IC-3, L³ is —C(O)—, L⁴ is a covalent bond, R⁶ is alkoxyalkyl, and Y is a substituted or unsubstituted purinyl.

In another embodiment of the compounds of formula IC-3, L³ is —CH₂—, L⁴ is —C(R⁴)₂—, wherein R⁴ is as defined herein, R⁶ is alkoxyalkyl, and Y is a substituted or unsubstituted furanyl.

In another embodiment of the compounds of formula IC-3, L³ is —CH(CH₃)—, L⁴ is —C(R⁴)₂— wherein R⁴ is as defined herein, R⁶ is alkoxyalkyl, and Y is a substituted or unsubstituted furanyl.

In another embodiment of the compounds of formula IC-3, L³ is —C(O)—, L⁴ is —C(R⁴)₂— wherein R⁴ is as defined herein, R⁶ is alkoxyalkyl, and Y is a substituted or unsubstituted furanyl.

In another embodiment of the compounds of formula IC-3, L³ is —C(R⁴)₂—, L⁴ is —CH(CH₃)— wherein R⁴ is as defined herein, R⁶ is alkoxyalkyl, and Y is a substituted or unsubstituted furanyl.

In another embodiment of the compounds of formula IC-3, L³ is —CH₂—, L⁴ is —CH(CH₃)—, R⁶ is alkoxyalkyl, and Y is a substituted or unsubstituted furanyl.

In another embodiment of the compounds of formula IC-3, L³ is —CH(CH₃)—, L⁴ is —CH(CH₃)—, R⁶ is alkoxyalkyl, and Y is a substituted or unsubstituted furanyl.

In another embodiment of the compounds of formula IC-3, L³ is —C(O)—, L⁴ is —CH(CH₃)—, R⁶ is alkoxyalkyl, and Y is a substituted or unsubstituted furanyl.

In another embodiment of the compounds of formula IC-3, L³ is —C(R⁴)₂— wherein R⁴ is as defined herein, L⁴ is a covalent bond, R⁶ is alkoxyalkyl, and Y is a substituted or unsubstituted furanyl.

In another embodiment of the compounds of formula IC-3, L³ is —CH₂—, L⁴ is a covalent bond, R⁶ is alkoxyalkyl, and Y is a substituted or unsubstituted furanyl.

In another embodiment of the compounds of formula IC-3, L³ is —CH(CH₃)—, L⁴ is a covalent bond, R⁶ is alkoxyalkyl, and Y is a substituted or unsubstituted furanyl.

In another embodiment of the compounds of formula IC-3, L³ is —C(O)—, L⁴ is a covalent bond, R⁶ is alkoxyalkyl, and Y is a substituted or unsubstituted furanyl.

In another embodiment of the compounds of formula IC-3, L³ is —CH₂—, L¹ is —C(R⁴)₂—, wherein R⁴ is as defined herein, R⁶ is alkoxyalkyl, and Y is a substituted or unsubstituted pyrrolyl.

In another embodiment of the compounds of formula IC-3, L³ is —CH(CH₃)—, L⁴ is —C(R⁴)₂— wherein R⁴ is as defined herein, R⁶ is alkoxyalkyl, and Y is a substituted or unsubstituted pyrrolyl.

In another embodiment of the compounds of formula IC-3, L³ is —C(O)—, L⁴ is —C(R⁴)₂— wherein R⁴ is as defined herein, R⁶ is alkoxyalkyl, and Y is a substituted or unsubstituted pyrrolyl.

In another embodiment of the compounds of formula IC-3, L³ is —C(R⁴)₂—, L⁴ is —CH(CH₃)— wherein R⁴ is as defined herein, R⁶ is alkoxyalkyl, and Y is a substituted or unsubstituted pyrrolyl.

In another embodiment of the compounds of formula IC-3, L³ is —CH₂—, L⁴ is —CH(CH₃)—, R⁶ is alkoxyalkyl, and Y is a substituted or unsubstituted pyrrolyl.

In another embodiment of the compounds of formula IC-3, L³ is —CH(CH₃)—, L⁴ is —CH(CH₃)—, R⁶ is alkoxyalkyl, and Y is a substituted or unsubstituted pyrrolyl.

In another embodiment of the compounds of formula IC-3, L³ is —C(O)—, L⁴ is —CH(CH₃)—, R⁶ is alkoxyalkyl, and Y is a substituted or unsubstituted pyrrolyl.

In another embodiment of the compounds of formula IC-3, L³ is —C(R⁴)₂— wherein R⁴ is as defined herein, L⁴ is a covalent bond, R⁶ is alkoxyalkyl, and Y is a substituted or unsubstituted pyrrolyl.

In another embodiment of the compounds of formula IC-3, L³ is —CH₂—, L⁴ is a covalent bond, R⁶ is alkoxyalkyl, and Y is a substituted or unsubstituted pyrrolyl.

In another embodiment of the compounds of formula IC-3, L³ is —CH(CH₃)—, L⁴ is a covalent bond, R⁶ is alkoxyalkyl, and Y is a substituted or unsubstituted pyrrolyl.

In another embodiment of the compounds of formula IC-3, L³ is —C(O)—, L⁴ is a covalent bond, R⁶ is alkoxyalkyl, and Y is a substituted or unsubstituted pyrrolyl.

In another embodiment of the compounds of formula IC-3, L³ is —CH₂—, L⁴ is —C(R⁴)₂—, wherein R⁴ is as defined herein, R⁶ is alkoxyalkyl, and Y is alkoxyalkyl.

In another embodiment of the compounds of formula IC-3, L³ is —CH(CH₃)—, L⁴ is —C(R⁴)₂— wherein R⁴ is as defined herein, R⁶ is alkoxyalkyl, and Y is alkoxyalkyl.

In another embodiment of the compounds of formula IC-3, L³ is —C(O)—, L⁴ is —C(R⁴)₂— wherein R⁴ is as defined herein, R⁶ is alkoxyalkyl, and Y is alkoxyalkyl.

In another embodiment of the compounds of formula IC-3, L³ is —C(R⁴)₂—, L⁴ is —CH(CH₃)— wherein R⁴ is as defined herein, R⁶ is alkoxyalkyl, and Y is alkoxyalkyl.

In another embodiment of the compounds of formula IC-3, L³ is —CH₂—, L⁴ is —CH(CH₃)—, R⁶ is alkoxyalkyl, and Y is alkoxyalkyl.

In another embodiment of the compounds of formula IC-3, L³ is —CH(CH₃)—, L⁴ is —CH(CH₃)—, R⁶ is alkoxyalkyl, and Y is alkoxyalkyl.

In another embodiment of the compounds of formula IC-3, L³ is —C(O)—, L⁴ is —CH(CH₃)—, R⁶ is alkoxyalkyl, and Y is alkoxyalkyl.

In another embodiment of the compounds of formula IC-3, L³ is —C(R⁴)₂— wherein R⁴ is as defined herein, L⁴ is a covalent bond, R⁶ is alkoxyalkyl, and Y is alkoxyalkyl.

In another embodiment of the compounds of formula IC-3, L³ is —CH₂—, L⁴ is a covalent bond, R⁶ is alkoxyalkyl, and Y is alkoxyalkyl.

In another embodiment of the compounds of formula IC-3, L³ is —CH(CH₃)—, L⁴ is a covalent bond, R⁶ is alkoxyalkyl, and Y is alkoxyalkyl.

In another embodiment of the compounds of formula IC-3, L³ is —C(O)—, L⁴ is a covalent bond, R⁶ is alkoxyalkyl, and Y is alkoxyalkyl.

In another embodiment of the compounds of formula IC-3, R⁶ and -L⁴-Y, together with the nitrogen atom to which they are attached, form a substituted or unsubstituted heterocyclyl.

In another embodiment of the compounds of formula IC-3, R⁶ and -L⁴-Y, together with the nitrogen atom to which they are attached, form a substituted or unsubstituted piperidyl.

In another embodiment of the compounds of formula IC-3, R⁶ and -L⁴-Y, together with the nitrogen atom to which they are attached, form a substituted or unsubstituted piperazinyl.

In another embodiment of the compounds of formula IC-3, R⁶ and -L⁴-Y, together with the nitrogen atom to which they are attached, form a substituted or unsubstituted pyrrolyl.

In another embodiment of the compounds of formula IC-3, R⁶ and -L⁴-Y, together with the nitrogen atom to which they are attached, form a substituted or unsubstituted morpholinyl.

In another embodiment of the compounds of formula IC-3, R⁶ and -L⁴-Y, together with the nitrogen atom to which they are attached, form a substituted or unsubstituted heterocyclyl, and L³ is —C(R⁴)₂— wherein R⁴ is as defined or described herein.

In another embodiment of the compounds of formula IC-3, R⁶ and -L⁴-Y, together with the nitrogen atom to which they are attached, form a substituted or unsubstituted piperidyl, and L³ is —C(R⁴)₂— wherein R⁴ is as defined or described herein.

In another embodiment of the compounds of formula IC-3, R⁶ and -L⁴-Y, together with the nitrogen atom to which they are attached, form a substituted or unsubstituted piperazinyl, and L³ is —C(R⁴)₂— wherein R⁴ is as defined or described herein.

In another embodiment of the compounds of formula IC-3, R⁶ and -L⁴-Y, together with the nitrogen atom to which they are attached, form a substituted or unsubstituted pyrrolyl, and L³ is —C(R⁴)₂— wherein R⁴ is as defined or described herein.

In another embodiment of the compounds of formula IC-3, R⁶ and -L⁴-Y, together with the nitrogen atom to which they are attached, form a substituted or unsubstituted morpholinyl, and L³ is —C(R⁴)₂— wherein R⁴ is as defined or described herein.

In another embodiment of the compounds of formula IC-3, R⁶ and -L⁴-Y, together with the nitrogen atom to which they are attached, form a substituted or unsubstituted heterocyclyl, and L³ is —CH₂— wherein R⁴ is as defined or described herein.

In another embodiment of the compounds of formula IC-3, R⁶ and -L⁴-Y, together with the nitrogen atom to which they are attached, form a substituted or unsubstituted piperidyl, and L³ is —CH₂— wherein R⁴ is as defined or described herein.

In another embodiment of the compounds of formula IC-3, R⁶ and -L⁴-Y, together with the nitrogen atom to which they are attached, form a substituted or unsubstituted piperazinyl, and L³ is —CH₂— wherein R⁴ is as defined or described herein.

In another embodiment of the compounds of formula IC-3, R⁶ and -L⁴-Y, together with the nitrogen atom to which they are attached, form a substituted or unsubstituted pyrrolyl, and L³ is —CH₂— wherein R⁴ is as defined or described herein.

In another embodiment of the compounds of formula IC-3, R⁶ and -L⁴-Y, together with the nitrogen atom to which they are attached, form a substituted or unsubstituted morpholinyl, and L³ is —CH₂— wherein R⁴ is as defined or described herein.

In another embodiment of the compounds of formula IC-3, R⁶ and -L⁴-Y, together with the nitrogen atom to which they are attached, form a substituted or unsubstituted heterocyclyl, and L³ is —CH(CH₃)— wherein R⁴ is as defined or described herein.

In another embodiment of the compounds of formula IC-3, R⁶ and -L⁴-Y, together with the nitrogen atom to which they are attached, form a substituted or unsubstituted piperidyl, and L³ is —CH(CH₃)— wherein R⁴ is as defined or described herein.

In another embodiment of the compounds of formula IC-3, R⁶ and -L⁴-Y, together with the nitrogen atom to which they are attached, form a substituted or unsubstituted piperazinyl, and L³ is —CH(CH₃)— wherein R⁴ is as defined or described herein.

In another embodiment of the compounds of formula IC-3, R⁶ and -L⁴-Y, together with the nitrogen atom to which they are attached, form a substituted or unsubstituted pyrrolyl, and L³ is —CH(CH₃)— wherein R⁴ is as defined or described herein.

In another embodiment of the compounds of formula IC-3, R⁶ and -L⁴-Y, together with the nitrogen atom to which they are attached, form a substituted or unsubstituted morpholinyl, and L³ is —CH(CH₃)— wherein R⁴ is as defined or described herein.

In another embodiment of the compounds of formula IC-3, R⁶ and -L⁴-Y, together with the nitrogen atom to which they are attached, form a substituted or unsubstituted heterocyclyl, and L³ is —C(O)— wherein R⁴ is as defined or described herein.

In another embodiment of the compounds of formula IC-3, R⁶ and -L⁴-Y, together with the nitrogen atom to which they are attached, form a substituted or unsubstituted piperidyl, and L³ is —C(O)— wherein R⁴ is as defined or described herein.

In another embodiment of the compounds of formula IC-3, R⁶ and -L⁴-Y, together with the nitrogen atom to which they are attached, form a substituted or unsubstituted piperazinyl, and L³ is —C(O)— wherein R⁴ is as defined or described herein.

In another embodiment of the compounds of formula IC-3, R⁶ and -L⁴-Y, together with the nitrogen atom to which they are attached, form a substituted or unsubstituted pyrrolyl, and L³ is —C(O)— wherein R⁴ is as defined or described herein.

In another embodiment of the compounds of formula IC-3, R⁶ and -L⁴-Y, together with the nitrogen atom to which they are attached, form a substituted or unsubstituted morpholinyl, and L³ is —C(O)— wherein R⁴ is as defined or described herein.

In another embodiment of the compounds of formula IC-3, L² is —C(O)O— and X is arylalkyl.

In another embodiment of the compounds of formula IC-3, L² is —C(O)O— and X is benzyl.

In another embodiment of the compounds of formula IC-3, L³ is —C(O)O—, X is arylalkyl, and R⁵ and R¹, together with the nitrogen atoms to which they are each attached, form a substituted or unsubstituted heterocyclyl ring.

In another embodiment of the compounds of formula IC-3, L² is —C(O)O—, X is benzyl, and R⁵ and R¹, together with the nitrogen atoms to which they are each attached, form a substituted or unsubstituted heterocyclyl ring.

In another embodiment of the compounds of formula IC-3, L² is a covalent bond, X is H, and R⁵ and R¹, together with the nitrogen atoms to which they are each attached, form a substituted or unsubstituted heterocyclyl ring.

In another embodiment of the compounds of formula IC-3, L² is —C(O)— and X is aminoalkyl.

In another embodiment of the compounds of formula IC-3, L² is —C(O)— and X is aminopropyl.

In another embodiment of the compounds of formula IC-3, L³ is —C(O)— and X is urethanylalkyl.

In another embodiment of the compounds of formula IC-3, L³ is —C(O)— and X is urethanylpropyl.

In another embodiment of the compounds of formula IC-3, L² is —C(O)—, X is aminoalkyl, and R⁵ and R¹, together with the nitrogen atoms to which they are each attached, form a substituted or unsubstituted heterocyclyl ring.

In another embodiment of the compounds of formula IC-3, L² is —C(O)—, X is aminopropyl, and R⁵ and R¹, together with the nitrogen atoms to which they are each attached, form a substituted or unsubstituted heterocyclyl ring.

In another embodiment of the compounds of formula IC-3, L³ is —C(O)—, X is urethanylalkyl, and R⁵ and R¹, together with the nitrogen atoms to which they are each attached, form a substituted or unsubstituted heterocyclyl ring.

In another embodiment of the compounds of formula IC-3, L³ is —C(O)—, X is urethanylpropyl, and R⁵ and R¹, together with the nitrogen atoms to which they are each attached, form a substituted or unsubstituted heterocyclyl ring.

In another embodiment of the compounds of formula IC-3, L² is a covalent bond, X is H, and R⁵ and R¹, together with the nitrogen atoms to which they are each attached, form a substituted or unsubstituted heterocyclyl ring.

In another embodiment of the compounds of formula IC-3, L⁴ is —S(O)₂— and Y is substituted or unsubstituted aryl.

In another embodiment of the compounds of formula IC-3, L⁴ is —S(O)₂— and Y is substituted or unsubstituted phenyl.

In another embodiment of the compounds of formula IC-3, L⁴ is —S(O)₂—, Y is substituted or unsubstituted aryl, and R⁶ is arylalkyl.

In another embodiment of the compounds of formula IC-3, L⁴ is —S(O)₂—, Y is substituted or unsubstituted aryl, and R⁶ is heteroarylalkyl.

In another embodiment of the compounds of formula IC-3, L⁴ is —S(O)₂—, Y is substituted or unsubstituted aryl, and R⁶ is pyridylalkyl.

In another embodiment of the compounds of formula IC-3, L⁴ is —S(O)₂—, Y is substituted or unsubstituted phenyl and R⁶ is arylalkyl.

In another embodiment of the compounds of formula IC-3, L⁴ is —S(O)₂—, Y is substituted or unsubstituted phenyl and R⁶ is heteroarylalkyl.

In another embodiment of the compounds of formula IC-3, L⁴ is —S(O)₂—, Y is substituted or unsubstituted phenyl and R⁶ is pyridylalkyl.

The compounds of formulae I, IC, IC-1, IC-2, and IC-3 do not include the compounds disclosed in WO 2006/027211 (herein incorporated by reference in its entirety), specifically:

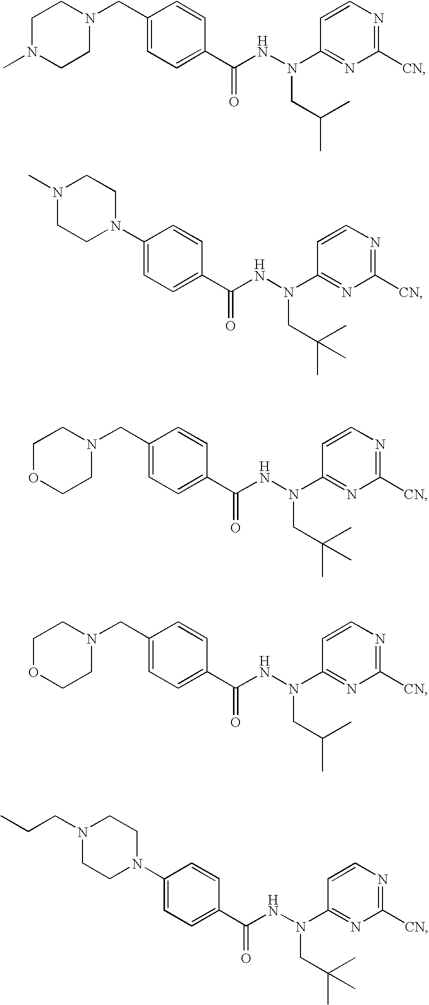

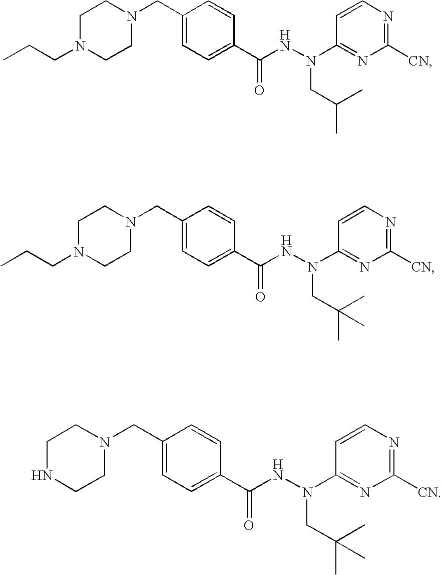

The compounds of formulae I, IC, IC-2, and IC-3 also do not include the compounds disclosed in WO 2004/056784 (herein incorporated by reference in its entirety), specifically, compounds of the following general formula:

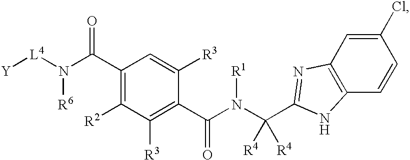

wherein R¹, R², R³, R⁴, R⁶, L⁴, and Y are as defined herein.

In another embodiment, the compounds of the present invention, or pharmaceutically acceptable salts, solvates, prodrugs, tautomers, or esters thereof, have the structure of formula (ID):

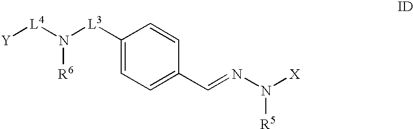

wherein Y-L⁴-N(R⁶)—, L³, X and R⁵ are as defined herein.

In another embodiment of the compounds of formula ID, R⁴, R⁵ and R⁶ are H; X is selected from the group consisting of substituted or unsubstituted phenyl, and substituted or unsubstituted pyrimidyl; Y is selected from the group consisting of substituted or unsubstituted phenyl, substituted or unsubstituted pyridyl, and substituted or unsubstituted pyrimidyl.

In another embodiment of the compounds of formula ID, X is substituted or unsubstituted aryl.

In another embodiment of the compounds of formula ID, X is substituted or unsubstituted phenyl.

In another embodiment of the compounds of formula ID, X is substituted or unsubstituted heteroaryl.

In another embodiment of the compounds of formula ID, X is substituted or unsubstituted pyridyl.

In another embodiment of the compounds of formula ID, X is substituted or unsubstituted pyrimidyl.

In another embodiment of the compounds of formula ID, X is substituted or unsubstituted indolyl.

In another embodiment of the compounds of formula ID, X is substituted or unsubstituted quinolyl.

In another embodiment of the compounds of formula ID, X is substituted or unsubstituted purinyl.

In another embodiment of the compounds of formula ID, X is substituted or unsubstituted thienyl.

In another embodiment of the compounds of formula ID, X is substituted or unsubstituted furanyl.

In another embodiment of the compounds of formula ID, X is substituted or unsubstituted pyrrolyl.

In another embodiment of the compounds of formula ID, X is substituted or unsubstituted benzimidazolyl.

In another embodiment of the compounds of formula ID, R⁵ is H.

In another embodiment of the compounds of formula ID, R⁵ is alkyl.

In another embodiment of the compounds of formula ID, R⁵ is methyl.

In another embodiment of the compounds of formula ID, R⁵ is ethyl.

In another embodiment of the compounds of formula ID, R⁵ is substituted or unsubstituted benzyl.

In another embodiment of the compounds of formula ID, X is substituted or unsubstituted aryl, and R⁵ is H.

In another embodiment of the compounds of formula ID, X is substituted or unsubstituted phenyl, and R⁵ is H.

In another embodiment of the compounds of formula ID, X is substituted or unsubstituted heteroaryl, and R⁵ is H.

In another embodiment of the compounds of formula ID, X is substituted or unsubstituted pyridyl, and R⁵ is H.

In another embodiment of the compounds of formula ID, X is substituted or unsubstituted pyrimidyl, and R⁵ is H.

In another embodiment of the compounds of formula ID, X is substituted or unsubstituted indolyl, and R⁵ is H.

In another embodiment of the compounds of formula ID, X is substituted or unsubstituted quinolyl, and R⁵ is H.

In another embodiment of the compounds of formula ID, X is substituted or unsubstituted purinyl, and R⁵ is H.

In another embodiment of the compounds of formula ID, X is substituted or unsubstituted thienyl, and R⁵ is H.

In another embodiment of the compounds of formula ID, X is substituted or unsubstituted furanyl, and R⁵ is H.

In another embodiment of the compounds of formula ID, X is substituted or unsubstituted pyrrolyl, and R⁵ is H.

In another embodiment of the compounds of formula ID, X is substituted or unsubstituted benzimidazolyl, and R⁵ is H.

In another embodiment of the compounds of formula ID, X is substituted or unsubstituted aryl, and R⁵ is alkyl.

In another embodiment of the compounds of formula ID, X is substituted or unsubstituted phenyl, and R⁵ is alkyl.

In another embodiment of the compounds of formula ID, X is substituted or unsubstituted heteroaryl, and R⁵ is alkyl.

In another embodiment of the compounds of formula ID, X is substituted or unsubstituted pyridyl, and R⁵ is alkyl.

In another embodiment of the compounds of formula ID, X is substituted or unsubstituted pyrimidyl, and R⁵ is alkyl.

In another embodiment of the compounds of formula ID, X is substituted or unsubstituted indolyl, and R⁵ is alkyl.

In another embodiment of the compounds of formula ID, X is substituted or unsubstituted quinolyl, and R⁵ is alkyl.

In another embodiment of the compounds of formula ID, X is substituted or unsubstituted purinyl, and R⁵ is alkyl.

In another embodiment of the compounds of formula ID, X is substituted or unsubstituted thienyl, and R⁵ is alkyl.

In another embodiment of the compounds of formula ID, X is substituted or unsubstituted furanyl, and R⁵ is alkyl.

In another embodiment of the compounds of formula ID, X is substituted or unsubstituted pyrrolyl, and R⁵ is alkyl.

In another embodiment of the compounds of formula ID, X is substituted or unsubstituted benzimidazolyl, and R⁵ is alkyl.

In another embodiment of the compounds of formula ID, X is substituted or unsubstituted aryl, and R⁵ is methyl.

In another embodiment of the compounds of formula ID, X is substituted or unsubstituted phenyl, and R⁵ is methyl.

In another embodiment of the compounds of formula ID, X is substituted or unsubstituted heteroaryl, and R⁵ is methyl.

In another embodiment of the compounds of formula ID, X is substituted or unsubstituted pyridyl, and R⁵ is methyl.

In another embodiment of the compounds of formula ID, X is substituted or unsubstituted pyrimidyl, and R⁵ is methyl.

In another embodiment of the compounds of formula ID, X is substituted or unsubstituted indolyl, and R⁵ is methyl.

In another embodiment of the compounds of formula ID, X is substituted or unsubstituted quinolyl, and R⁵ is methyl.

In another embodiment of the compounds of formula ID, X is substituted or unsubstituted purinyl, and R⁵ is methyl.

In another embodiment of the compounds of formula ID, X is substituted or unsubstituted thienyl, and R⁵ is methyl.

In another embodiment of the compounds of formula ID, X is substituted or unsubstituted furanyl, and R⁵ is methyl.

In another embodiment of the compounds of formula ID, X is substituted or unsubstituted pyrrolyl, and R⁵ is methyl.

In another embodiment of the compounds of formula ID, X is substituted or unsubstituted benzimidazolyl, and R⁵ is methyl.

In another embodiment of the compounds of formula ID, X is substituted or unsubstituted aryl, and R⁵ is ethyl.

In another embodiment of the compounds of formula ID, X is substituted or unsubstituted phenyl, and R⁵ is ethyl.

In another embodiment of the compounds of formula ID, X is substituted or unsubstituted heteroaryl, and R⁵ is ethyl.

In another embodiment of the compounds of formula ID, X is substituted or unsubstituted pyridyl, and R⁵ is ethyl.

In another embodiment of the compounds of formula ID, X is substituted or unsubstituted pyrimidyl, and R⁵ is ethyl.

In another embodiment of the compounds of formula ID, X is substituted or unsubstituted indolyl, and R⁵ is ethyl.

In another embodiment of the compounds of formula ID, X is substituted or unsubstituted quinolyl, and R⁵ is ethyl.

In another embodiment of the compounds of formula ID, X is substituted or unsubstituted purinyl, and R⁵ is ethyl.

In another embodiment of the compounds of formula ID, X is substituted or unsubstituted thienyl, and R⁵ is ethyl.

In another embodiment of the compounds of formula ID, X is substituted or unsubstituted furanyl, and R⁵ is ethyl.

In another embodiment of the compounds of formula ID, X is substituted or unsubstituted pyrrolyl, and R⁵ is ethyl.

In another embodiment of the compounds of formula ID, X is substituted or unsubstituted benzimidazolyl, and R⁵ is ethyl.

In another embodiment of the compounds of formula ID, X is substituted or unsubstituted aryl, and R⁵ is substituted or unsubstituted benzyl.

In another embodiment of the compounds of formula ID, X is substituted or unsubstituted phenyl, and R⁵ is substituted or unsubstituted benzyl.

In another embodiment of the compounds of formula ID, X is substituted or unsubstituted heteroaryl, and R⁵ is substituted or unsubstituted benzyl.

In another embodiment of the compounds of formula ID, X is substituted or unsubstituted pyridyl, and R⁵ is substituted or unsubstituted benzyl.

In another embodiment of the compounds of formula ID, X is substituted or unsubstituted pyrimidyl, and R⁵ is substituted or unsubstituted benzyl.

In another embodiment of the compounds of formula ID, X is substituted or unsubstituted indolyl, and R⁵ is substituted or unsubstituted benzyl.

In another embodiment of the compounds of formula ID, X is substituted or unsubstituted quinolyl, and R⁵ is substituted or unsubstituted benzyl.

In another embodiment of the compounds of formula ID, X is substituted or unsubstituted purinyl, and R⁵ is substituted or unsubstituted benzyl.

In another embodiment of the compounds of formula ID, X is substituted or unsubstituted thienyl, and R⁵ is substituted or unsubstituted benzyl.

In another embodiment of the compounds of formula ID, X is substituted or unsubstituted furanyl, and R⁵ is substituted or unsubstituted benzyl.

In another embodiment of the compounds of formula ID, X is substituted or unsubstituted pyrrolyl, and R⁵ is substituted or unsubstituted benzyl.

In another embodiment of the compounds of formula ID, X is substituted or unsubstituted benzimidazolyl, and R⁵ is substituted or unsubstituted benzyl.

In another embodiment of the compounds of formula ID, L³ is —C(R⁴)₂—.

In another embodiment of the compounds of formula ID, L³ is —CH₂—.

In another embodiment of the compounds of formula ID, L³ is —CH(CH₃)—.

In another embodiment of the compounds of formula ID, L³ is —C(O)—.

In another embodiment of the compounds of formula ID, L³ is —S(O)₂—.

In another embodiment of the compounds of formula ID, L⁴ is a covalent bond.

In another embodiment of the compounds of formula ID, L⁴ is —C(R⁴)₂—.

In another embodiment of the compounds of formula ID, L⁴ is —CH₂—.

In another embodiment of the compounds of formula ID, L⁴ is —CH(CH₃)—.

In another embodiment of the compounds of formula ID, L³ is —C(R⁴)₂—, and L⁴ is a covalent bond.

In another embodiment of the compounds of formula ID, L³ is —CH₂—, and L⁴ is a covalent bond.

In another embodiment of the compounds of formula ID, L³ is —CH(CH₃)—, and L⁴ is a covalent bond.

In another embodiment of the compounds of formula ID, L³ is —C(O)—, and L⁴ is a covalent bond.

In another embodiment of the compounds of formula ID, L³ is —S(O)₂—, and L⁴ is a covalent bond.

In another embodiment of the compounds of formula ID, L³ is —C(R⁴)₂—, and L⁴ is —C(R⁴)₂—.

In another embodiment of the compounds of formula ID, L³ is —CH₂—, and L⁴ is —C(R⁴)₂—.

In another embodiment of the compounds of formula ID, L³ is —CH(CH₃)—, and L⁴ is —C(R⁴)₂—.

In another embodiment of the compounds of formula ID, L³ is —C(O)—, and L⁴ is —C(R⁴)₂—.

In another embodiment of the compounds of formula ID, L³ is —S(O)₂—, and L⁴ is —C(R⁴)₂—.

In another embodiment of the compounds of formula ID, L³ is —C(R⁴)₂—, and L⁴ is —CH₂—.

In another embodiment of the compounds of formula ID, L³ is —CH₂—, and L⁴ is —CH₂—

In another embodiment of the compounds of formula ID, L³ is —CH(CH₃)—, and L⁴ is —CH₂—.

In another embodiment of the compounds of formula ID, L³ is —C(O)—, and L⁴ is —CH₂—.

In another embodiment of the compounds of formula ID, L³ is —S(O)₂—, and L⁴ is —CH₂—.

In another embodiment of the compounds of formula ID, L³ is —C(R⁴)₂—, and L⁴ is —CH(CH₃)—.

In another embodiment of the compounds of formula ID, L³ is —CH₂—, and L⁴ is —CH(CH₃)—.

In another embodiment of the compounds of formula ID, L³ is —CH(CH₃)—, and L⁴ is —CH(CH₃)—.

In another embodiment of the compounds of formula ID, L³ is —C(O)—, and L⁴ is —CH(CH₃)—.

In another embodiment of the compounds of formula ID, L³ is —S(O)₂—, and L⁴ is —CH(CH₃)—.

In another embodiment of the compounds of formula ID, Y is substituted or unsubstituted aryl.

In another embodiment of the compounds of formula ID, Y is substituted or unsubstituted phenyl.

In another embodiment of the compounds of formula ID, Y is substituted or unsubstituted heteroaryl.

In another embodiment of the compounds of formula ID, Y is substituted or unsubstituted pyridyl.

In another embodiment of the compounds of formula ID, Y is substituted or unsubstituted pyrimidyl.

In another embodiment of the compounds of formula ID, Y is substituted or unsubstituted thienyl.

In another embodiment of the compounds of formula ID, Y is substituted or unsubstituted quinolyl.

In another embodiment of the compounds of formula ID, Y is substituted or unsubstituted furanyl.

In another embodiment of the compounds of formula ID, Y is substituted or unsubstituted indolyl.

In another embodiment of the compounds of formula ID, Y is substituted or unsubstituted puranyl.

In another embodiment of the compounds of formula ID, Y is substituted or unsubstituted pyrrolyl.

In another embodiment of the compounds of formula ID, Y is substituted or unsubstituted benzimidazolyl.

In another embodiment of the compounds of formula ID, L³ is —C(R⁴)₂—, and Y is substituted or unsubstituted aryl.

In another embodiment of the compounds of formula ID, L³ is —CH₂—, and Y is substituted or unsubstituted aryl.

In another embodiment of the compounds of formula ID, L³ is —CH(CH₃)—, and Y is substituted or unsubstituted aryl.

In another embodiment of the compounds of formula ID, L³ is —C(O)—, and Y is substituted or unsubstituted aryl.

In another embodiment of the compounds of formula ID, L³ is —S(O)₂—, and Y is substituted or unsubstituted aryl.

In another embodiment of the compounds of formula ID, L⁴ is a covalent bond, and Y is substituted or unsubstituted aryl.

In another embodiment of the compounds of formula ID, L⁴ is —C(R⁴)₂—, and Y is substituted or unsubstituted aryl.

In another embodiment of the compounds of formula ID, L⁴ is —CH₂—, and Y is substituted or unsubstituted aryl.

In another embodiment of the compounds of formula ID, L⁴ is —CH(CH₃)—, and Y is substituted or unsubstituted aryl.

In another embodiment of the compounds of formula ID, L³ is —C(R⁴)₂—, L⁴ is a covalent bond, and Y is substituted or unsubstituted aryl.

In another embodiment of the compounds of formula ID, L³ is —CH₂—, L⁴ is a covalent bond, and Y is substituted or unsubstituted aryl.

In another embodiment of the compounds of formula ID, L³ is —CH(CH₃)—, L⁴ is a covalent bond, and Y is substituted or unsubstituted aryl.

In another embodiment of the compounds of formula ID, L³ is —C(O)—, L⁴ is a covalent bond, and Y is substituted or unsubstituted aryl.

In another embodiment of the compounds of formula ID, L³ is —S(O)₂—, L⁴ is a covalent bond, and Y is substituted or unsubstituted aryl.

In another embodiment of the compounds of formula ID, L³ is —C(R⁴)₂—, L⁴ is —C(R⁴)₂—, and Y is substituted or unsubstituted aryl.

In another embodiment of the compounds of formula ID, L³ is —CH₂—, L⁴ is —C(R⁴)₂—, and Y is substituted or unsubstituted aryl.

In another embodiment of the compounds of formula ID, L³ is —CH(CH₃)—, L⁴ is —C(R⁴)₂—, and Y is substituted or unsubstituted aryl.

In another embodiment of the compounds of formula ID, L³ is —C(O)—, L⁴ is —C(R⁴)₂—, and Y is substituted or unsubstituted aryl.

In another embodiment of the compounds of formula ID, L³ is —S(O)₂—, L⁴ is —C(R⁴)₂—, and Y is substituted or unsubstituted aryl.

In another embodiment of the compounds of formula ID, L³ is —C(R⁴)₂—, L⁴ is —CH₂—, and Y is substituted or unsubstituted aryl.

In another embodiment of the compounds of formula ID, L³ is —CH₂—, L⁴ is —CH₂—, and Y is substituted or unsubstituted aryl.

In another embodiment of the compounds of formula ID, L³ is —CH(CH₃)—, L⁴ is —CH₂—, and Y is substituted or unsubstituted aryl.

In another embodiment of the compounds of formula ID, L³ is —C(O)—, L⁴ is —CH₂—, and Y is substituted or unsubstituted aryl.

In another embodiment of the compounds of formula ID, L³ is —S(O)₂—, L⁴ is —CH₂—, and Y is substituted or unsubstituted aryl.

In another embodiment of the compounds of formula ID, L³ is —C(R⁴)₂—, L⁴ is —CH(CH₃)—, and Y is substituted or unsubstituted aryl.

In another embodiment of the compounds of formula ID, L³ is —CH₂—, L⁴ is —CH(CH₃)—, and Y is substituted or unsubstituted aryl.

In another embodiment of the compounds of formula ID, L³ is —CH(CH₃)—, L⁴ is —CH(CH₃)—, and Y is substituted or unsubstituted aryl.

In another embodiment of the compounds of formula ID, L³ is —C(O)—, L⁴ is —CH(CH₃)—, and Y is substituted or unsubstituted aryl.

In another embodiment of the compounds of formula ID, L³ is —S(O)₂—, L⁴ is —CH(CH₃)—, and Y is substituted or unsubstituted aryl.

In another embodiment of the compounds of formula ID, L³ is —C(R⁴)₂—, and Y is substituted or unsubstituted phenyl.

In another embodiment of the compounds of formula ID, L³ is —CH₂—, and Y is substituted or unsubstituted phenyl.

In another embodiment of the compounds of formula ID, L³ is —CH(CH₃)—, and Y is substituted or unsubstituted phenyl.

In another embodiment of the compounds of formula ID, L³ is —C(O)—, and Y is substituted or unsubstituted phenyl.

In another embodiment of the compounds of formula ID, L³ is —S(O)₂—, and Y is substituted or unsubstituted phenyl.

In another embodiment of the compounds of formula ID, L⁴ is a covalent bond, and Y is substituted or unsubstituted phenyl.

In another embodiment of the compounds of formula ID, L⁴ is —C(R⁴)₂—, and Y is substituted or unsubstituted phenyl.

In another embodiment of the compounds of formula ID, L⁴ is —CH₂—, and Y is substituted or unsubstituted phenyl.

In another embodiment of the compounds of formula ID, L⁴ is —CH(CH₃)—, and Y is substituted or unsubstituted phenyl.

In another embodiment of the compounds of formula ID, L³ is —C(R⁴)₂—, L⁴ is a covalent bond, and Y is substituted or unsubstituted phenyl.

In another embodiment of the compounds of formula ID, L³ is —CH₂—, L⁴ is a covalent bond, and Y is substituted or unsubstituted phenyl.

In another embodiment of the compounds of formula ID, L³ is —CH(CH₃)—, L⁴ is a covalent bond, and Y is substituted or unsubstituted phenyl.

In another embodiment of the compounds of formula ID, L³ is —C(O)—, L⁴ is a covalent bond, and Y is substituted or unsubstituted phenyl.

In another embodiment of the compounds of formula ID, L³ is —S(O)₂—, L⁴ is a covalent bond, and Y is substituted or unsubstituted phenyl.

In another embodiment of the compounds of formula ID, L³ is —C(R⁴)₂—, L⁴ is —C(R⁴)₂—, and Y is substituted or unsubstituted phenyl.

In another embodiment of the compounds of formula ID, L³ is —CH₂—, L⁴ is —C(R⁴)₂—, and Y is substituted or unsubstituted phenyl.

In another embodiment of the compounds of formula ID, L³ is —CH(CH₃)—, L⁴ is —C(R⁴)₂—, and Y is substituted or unsubstituted phenyl.

In another embodiment of the compounds of formula ID, L³ is —C(O)—, L⁴ is —C(R⁴)₂—, and Y is substituted or unsubstituted phenyl.

In another embodiment of the compounds of formula ID, L³ is —S(O)₂—, L⁴ is —C(R⁴)₂—, and Y is substituted or unsubstituted phenyl.

In another embodiment of the compounds of formula ID, L³ is —C(R⁴)₂—, L⁴ is —CH₂—, and Y is substituted or unsubstituted phenyl.

In another embodiment of the compounds of formula ID, L³ is —CH₂—, L⁴ is —CH₂—, and Y is substituted or unsubstituted phenyl.

In another embodiment of the compounds of formula ID, L³ is —CH(CH₃)—, L⁴ is —CH₂—, and Y is substituted or unsubstituted phenyl.

In another embodiment of the compounds of formula ID, L³ is —C(O)—, L⁴ is —CH₂—, and Y is substituted or unsubstituted phenyl.

In another embodiment of the compounds of formula ID, L³ is —S(O)₂—, L⁴ is —CH₂—, and Y is substituted or unsubstituted phenyl.

In another embodiment of the compounds of formula ID, L³ is —C(R⁴)₂—, L⁴ is —CH(CH₃)—, and Y is substituted or unsubstituted phenyl.

In another embodiment of the compounds of formula ID, L³ is —CH₂—, L⁴ is —CH(CH₃)—, and Y is substituted or unsubstituted phenyl.

In another embodiment of the compounds of formula ID, L³ is —CH(CH₃)—, L⁴ is —CH(CH₃)—, and Y is substituted or unsubstituted phenyl.

In another embodiment of the compounds of formula ID, L³ is —C(O)—, L⁴ is —CH(CH₃)—, and Y is substituted or unsubstituted phenyl.

In another embodiment of the compounds of formula ID, L³ is —S(O)₂—, L⁴ is —CH(CH₃)—, and Y is substituted or unsubstituted phenyl.

In another embodiment of the compounds of formula ID, L³ is —C(R⁴)₂—, and Y is substituted or unsubstituted heteroaryl.

In another embodiment of the compounds of formula ID, L³ is —CH₂—, and Y is substituted or unsubstituted heteroaryl.

In another embodiment of the compounds of formula ID, L³ is —CH(CH₃)—, and Y is substituted or unsubstituted heteroaryl.

In another embodiment of the compounds of formula ID, L³ is —C(O)—, and Y is substituted or unsubstituted heteroaryl.

In another embodiment of the compounds of formula ID, L³ is —S(O)₂—, and Y is substituted or unsubstituted heteroaryl.

In another embodiment of the compounds of formula ID, L⁴ is a covalent bond, and Y is substituted or unsubstituted heteroaryl.

In another embodiment of the compounds of formula ID, L⁴ is —C(R⁴)₂—, and Y is substituted or unsubstituted heteroaryl.

In another embodiment of the compounds of formula ID, L⁴ is —CH₂—, and Y is substituted or unsubstituted heteroaryl.

In another embodiment of the compounds of formula ID, L⁴ is —CH(CH₃)—, and Y is substituted or unsubstituted heteroaryl.

In another embodiment of the compounds of formula ID, L³ is —C(R⁴)₂—, L⁴ is a covalent bond, and Y is substituted or unsubstituted heteroaryl.

In another embodiment of the compounds of formula ID, L³ is —CH₂—, L⁴ is a covalent bond, and Y is substituted or unsubstituted heteroaryl.

In another embodiment of the compounds of formula ID, L³ is —CH(CH₃)—, L⁴ is a covalent bond, and Y is substituted or unsubstituted heteroaryl.

In another embodiment of the compounds of formula ID, L³ is —C(O)—, L⁴ is a covalent bond, and Y is substituted or unsubstituted heteroaryl.

In another embodiment of the compounds of formula ID, L³ is —S(O)₂—, L⁴ is a covalent bond, and Y is substituted or unsubstituted heteroaryl.

In another embodiment of the compounds of formula ID, L³ is —C(R⁴)₂—, L⁴ is —C(R⁴)₂—, and Y is substituted or unsubstituted heteroaryl.

In another embodiment of the compounds of formula ID, L³ is —CH₂—, L⁴ is —C(R⁴)₂—, and Y is substituted or unsubstituted heteroaryl.

In another embodiment of the compounds of formula ID, L³ is —CH(CH₃)—, L⁴ is —C(R⁴)₂—, and Y is substituted or unsubstituted heteroaryl.

In another embodiment of the compounds of formula ID, L³ is —C(O)—, L⁴ is —C(R⁴)₂—, and Y is substituted or unsubstituted heteroaryl.

In another embodiment of the compounds of formula ID, L³ is —S(O)₂—, L⁴ is —C(R⁴)₂—, and Y is substituted or unsubstituted heteroaryl.

In another embodiment of the compounds of formula ID, L³ is —C(R⁴)₂—, L⁴ is —CH₂—, and Y is substituted or unsubstituted heteroaryl.

In another embodiment of the compounds of formula ID, L³ is —CH₂—, L⁴ is —CH₂—, and Y is substituted or unsubstituted heteroaryl.

In another embodiment of the compounds of formula ID, L³ is —CH(CH₃)—, L⁴ is —CH₂—, and Y is substituted or unsubstituted heteroaryl.

In another embodiment of the compounds of formula ID, L³ is —C(O)—, L⁴ is —CH₂—, and Y is substituted or unsubstituted heteroaryl.

In another embodiment of the compounds of formula ID, L³ is —S(O)₂—, L⁴ is —CH₂—, and Y is substituted or unsubstituted heteroaryl.

In another embodiment of the compounds of formula ID, L³ is —C(R⁴)₂—, L⁴ is —CH(CH₃)—, and Y is substituted or unsubstituted heteroaryl.

In another embodiment of the compounds of formula ID, L³ is —CH₂—, L⁴ is —CH(CH₃)—, and Y is substituted or unsubstituted heteroaryl.

In another embodiment of the compounds of formula ID, L³ is —CH(CH₃)—, L⁴ is —CH(CH₃)—, and Y is substituted or unsubstituted heteroaryl.

In another embodiment of the compounds of formula ID, L³ is —C(O)—, L⁴ is —CH(CH₃)—, and Y is substituted or unsubstituted heteroaryl.

In another embodiment of the compounds of formula ID, L³ is —S(O)₂—, L⁴ is —CH(CH₃)—, and Y is substituted or unsubstituted heteroaryl.

In another embodiment of the compounds of formula ID, L³ is —C(R⁴)₂—, and Y is substituted or unsubstituted pyridyl.

In another embodiment of the compounds of formula ID, L³ is —CH₂—, and Y is substituted or unsubstituted pyridyl.

In another embodiment of the compounds of formula ID, L³ is —CH(CH₃)—, and Y is substituted or unsubstituted pyridyl.

In another embodiment of the compounds of formula ID, L³ is —C(O)—, and Y is substituted or unsubstituted pyridyl.

In another embodiment of the compounds of formula ID, L³ is —S(O)₂—, and Y is substituted or unsubstituted pyridyl.

In another embodiment of the compounds of formula ID, L⁴ is a covalent bond, and Y is substituted or unsubstituted pyridyl.

In another embodiment of the compounds of formula ID, L⁴ is —C(R⁴)₂—, and Y is substituted or unsubstituted pyridyl.

In another embodiment of the compounds of formula ID, L⁴ is —CH₂—, and Y is substituted or unsubstituted pyridyl.

In another embodiment of the compounds of formula ID, L⁴ is —CH(CH₃)—, and Y is substituted or unsubstituted pyridyl.

In another embodiment of the compounds of formula ID, L³ is —C(R⁴)₂—, L⁴ is a covalent bond, and Y is substituted or unsubstituted pyridyl.

In another embodiment of the compounds of formula ID, L³ is —CH₂—, L⁴ is a covalent bond, and Y is substituted or unsubstituted pyridyl.

In another embodiment of the compounds of formula ID, L³ is —CH(CH₃)—, L⁴ is a covalent bond, and Y is substituted or unsubstituted pyridyl.

In another embodiment of the compounds of formula ID, L³ is —C(O)—, L⁴ is a covalent bond, and Y is substituted or unsubstituted pyridyl.

In another embodiment of the compounds of formula ID, L³ is —S(O)₂—, L⁴ is a covalent bond, and Y is substituted or unsubstituted pyridyl.

In another embodiment of the compounds of formula ID, L³ is —C(R⁴)₂—, L⁴ is —C(R⁴)₂—, and Y is substituted or unsubstituted pyridyl.

In another embodiment of the compounds of formula ID, L³ is —CH₂—, L⁴ is —C(R⁴)₂—, and Y is substituted or unsubstituted pyridyl.

In another embodiment of the compounds of formula ID, L³ is —CH(CH₃)—, L⁴ is —C(R⁴)₂—, and Y is substituted or unsubstituted pyridyl.

In another embodiment of the compounds of formula ID, L³ is —C(O)—, L⁴ is —C(R⁴)₂—, and Y is substituted or unsubstituted pyridyl.

In another embodiment of the compounds of formula ID, L³ is —S(O)₂—, L⁴ is —C(R⁴)₂—, and Y is substituted or unsubstituted pyridyl.

In another embodiment of the compounds of formula ID, L³ is —C(R⁴)₂—, L⁴ is —CH₂—, and Y is substituted or unsubstituted pyridyl.

In another embodiment of the compounds of formula ID, L³ is —CH₂—, L⁴ is —CH₂—, and Y is substituted or unsubstituted pyridyl.

In another embodiment of the compounds of formula ID, L³ is —CH(CH₃)—, L⁴ is —CH₂—, and Y is substituted or unsubstituted pyridyl.

In another embodiment of the compounds of formula ID, L³ is —C(O)—, L⁴ is —CH₂—, and Y is substituted or unsubstituted pyridyl.

In another embodiment of the compounds of formula ID, L³ is —S(O)₂—, L⁴ is —CH₂—, and Y is substituted or unsubstituted pyridyl.

In another embodiment of the compounds of formula ID, L³ is —C(R⁴)₂—, L⁴ is —CH(CH₃)—, and Y is substituted or unsubstituted pyridyl.

In another embodiment of the compounds of formula ID, L³ is —CH₂—, L⁴ is —CH(CH₃)—, and Y is substituted or unsubstituted pyridyl.

In another embodiment of the compounds of formula ID, L³ is —CH(CH₃)—, L⁴ is —CH(CH₃)—, and Y is substituted or unsubstituted pyridyl.

In another embodiment of the compounds of formula ID, L³ is —C(O)—, L⁴ is —CH(CH₃)—, and Y is substituted or unsubstituted pyridyl.

In another embodiment of the compounds of formula ID, L³ is —S(O)₂—, L⁴ is —CH(CH₃)—, and Y is substituted or unsubstituted pyridyl.

In another embodiment of the compounds of formula ID, L³ is —C(R⁴)₂—, and Y is substituted or unsubstituted pyrimidyl.

In another embodiment of the compounds of formula ID, L³ is —CH₂—, and Y is substituted or unsubstituted pyrimidyl.

In another embodiment of the compounds of formula ID, L³ is —CH(CH₃)—, and Y is substituted or unsubstituted pyrimidyl.

In another embodiment of the compounds of formula ID, L³ is —C(O)—, and Y is substituted or unsubstituted pyrimidyl.

In another embodiment of the compounds of formula ID, L³ is —S(O)₂—, and Y is substituted or unsubstituted pyrimidyl.

In another embodiment of the compounds of formula ID, L⁴ is a covalent bond, and Y is substituted or unsubstituted pyrimidyl.

In another embodiment of the compounds of formula ID, L⁴ is —C(R⁴)₂—, and Y is substituted or unsubstituted pyrimidyl.

In another embodiment of the compounds of formula ID, L⁴ is —CH₂—, and Y is substituted or unsubstituted pyrimidyl.

In another embodiment of the compounds of formula ID, L⁴ is —CH(CH₃)—, and Y is substituted or unsubstituted pyrimidyl.

In another embodiment of the compounds of formula ID, L³ is —C(R⁴)₂—, L⁴ is a covalent bond, and Y is substituted or unsubstituted pyrimidyl.

In another embodiment of the compounds of formula ID, L³ is —CH₂—, L⁴ is a covalent bond, and Y is substituted or unsubstituted pyrimidyl.

In another embodiment of the compounds of formula ID, L³ is —CH(CH₃)—, L⁴ is a covalent bond, and Y is substituted or unsubstituted pyrimidyl.

In another embodiment of the compounds of formula ID, L³ is —C(O)—, L⁴ is a covalent bond, and Y is substituted or unsubstituted pyrimidyl.

In another embodiment of the compounds of formula ID, L³ is —S(O)₂—, L⁴ is a covalent bond, and Y is substituted or unsubstituted pyrimidyl.

In another embodiment of the compounds of formula ID, L³ is —C(R⁴)₂—, L⁴ is —C(R⁴)₂—, and Y is substituted or unsubstituted pyrimidyl.

In another embodiment of the compounds of formula ID, L³ is —CH₂—, L⁴ is —C(R⁴)₂—, and Y is substituted or unsubstituted pyrimidyl.

In another embodiment of the compounds of formula ID, L³ is —CH(CH₃)—, L⁴ is —C(R⁴)₂—, and Y is substituted or unsubstituted pyrimidyl.

In another embodiment of the compounds of formula ID, L³ is —C(O)—, L⁴ is —C(R⁴)₂—, and Y is substituted or unsubstituted pyrimidyl.

In another embodiment of the compounds of formula ID, L³ is —S(O)₂—, L⁴ is —C(R⁴)₂—, and Y is substituted or unsubstituted pyrimidyl.

In another embodiment of the compounds of formula ID, L³ is —C(R⁴)₂—, L⁴ is —CH₂—, and Y is substituted or unsubstituted pyrimidyl.

In another embodiment of the compounds of formula ID, L³ is —CH₂—, L⁴ is —CH₂—, and Y is substituted or unsubstituted pyrimidyl.

In another embodiment of the compounds of formula ID, L³ is —CH(CH₃)—, L⁴ is —CH₂—, and Y is substituted or unsubstituted pyrimidyl.

In another embodiment of the compounds of formula ID, L³ is —C(O)—, L⁴ is —CH₂—, and Y is substituted or unsubstituted pyrimidyl.

In another embodiment of the compounds of formula ID, L³ is —S(O)₂—, L⁴ is —CH₂—, and Y is substituted or unsubstituted pyrimidyl.

In another embodiment of the compounds of formula ID, L³ is —C(R⁴)₂—, L⁴ is —CH(CH₃)—, and Y is substituted or unsubstituted pyrimidyl.

In another embodiment of the compounds of formula ID, L³ is —CH₂—, L⁴ is —CH(CH₃)—, and Y is substituted or unsubstituted pyrimidyl.

In another embodiment of the compounds of formula ID, L³ is —CH(CH₃)—, L⁴ is —CH(CH₃)—, and Y is substituted or unsubstituted pyrimidyl.

In another embodiment of the compounds of formula ID, L³ is —C(O)—, L⁴ is —CH(CH₃)—, and Y is substituted or unsubstituted pyrimidyl.

In another embodiment of the compounds of formula ID, L³ is —S(O)₂—, L⁴ is —CH(CH₃)—, and Y is substituted or unsubstituted pyrimidyl.

In another embodiment of the compounds of formula ID, L³ is —C(R⁴)₂—, and Y is substituted or unsubstituted thienyl.

In another embodiment of the compounds of formula ID, L³ is —CH₂—, and Y is substituted or unsubstituted thienyl.

In another embodiment of the compounds of formula ID, L³ is —CH(CH₃)—, and Y is substituted or unsubstituted thienyl.

In another embodiment of the compounds of formula ID, L³ is —C(O)—, and Y is substituted or unsubstituted thienyl.

In another embodiment of the compounds of formula ID, L³ is —S(O)₂—, and Y is substituted or unsubstituted thienyl.

In another embodiment of the compounds of formula ID, L⁴ is a covalent bond, and Y is substituted or unsubstituted thienyl.

In another embodiment of the compounds of formula ID, L⁴ is —C(R⁴)₂—, and Y is substituted or unsubstituted thienyl.

In another embodiment of the compounds of formula ID, L⁴ is —CH₂—, and Y is substituted or unsubstituted thienyl.

In another embodiment of the compounds of formula ID, L⁴ is —CH(CH₃)—, and Y is substituted or unsubstituted thienyl.

In another embodiment of the compounds of formula ID, L³ is —C(R⁴)₂—, L⁴ is a covalent bond, and Y is substituted or unsubstituted thienyl.

In another embodiment of the compounds of formula ID, L³ is —CH₂—, L⁴ is a covalent bond, and Y is substituted or unsubstituted thienyl.

In another embodiment of the compounds of formula ID, L³ is —CH(CH₃)—, L⁴ is a covalent bond, and Y is substituted or unsubstituted thienyl.

In another embodiment of the compounds of formula ID, L³ is —C(O)—, L⁴ is a covalent bond, and Y is substituted or unsubstituted thienyl.

In another embodiment of the compounds of formula ID, L³ is —S(O)₂—, L⁴ is a covalent bond, and Y is substituted or unsubstituted thienyl.

In another embodiment of the compounds of formula ID, L³ is —C(R⁴)₂—, L⁴ is —C(R⁴)₂—, and Y is substituted or unsubstituted thienyl.

In another embodiment of the compounds of formula ID, L³ is —CH₂—, L⁴ is —C(R⁴)₂—, and Y is substituted or unsubstituted thienyl.

In another embodiment of the compounds of formula ID, L³ is —CH(CH₃)—, L⁴ is —C(R⁴)₂—, and Y is substituted or unsubstituted thienyl.

In another embodiment of the compounds of formula ID, L³ is —C(O)—, L⁴ is —C(R⁴)₂—, and Y is substituted or unsubstituted thienyl.

In another embodiment of the compounds of formula ID, L³ is —S(O)₂—, L⁴ is —C(R⁴)₂—, and Y is substituted or unsubstituted thienyl.

In another embodiment of the compounds of formula ID, L³ is —C(R⁴)₂—, L⁴ is —CH₂—, and Y is substituted or unsubstituted thienyl.

In another embodiment of the compounds of formula ID, L³ is —CH₂—, L⁴ is —CH₂—, and Y is substituted or unsubstituted thienyl.

In another embodiment of the compounds of formula ID, L³ is —CH(CH₃)—, L⁴ is —CH₂—, and Y is substituted or unsubstituted thienyl.

In another embodiment of the compounds of formula ID, L³ is —C(O)—, L⁴ is —CH₂—, and Y is substituted or unsubstituted thienyl.

In another embodiment of the compounds of formula ID, L³ is —S(O)₂—, L⁴ is —CH₂—, and Y is substituted or unsubstituted thienyl.

In another embodiment of the compounds of formula ID, L³ is —C(R⁴)₂—, L⁴ is —CH(CH₃)—, and Y is substituted or unsubstituted thienyl.

In another embodiment of the compounds of formula ID, L³ is —CH₂—, L⁴ is —CH(CH₃)—, and Y is substituted or unsubstituted thienyl.

In another embodiment of the compounds of formula ID, L³ is —CH(CH₃)—, L⁴ is —CH(CH₃)—, and Y is substituted or unsubstituted thienyl.

In another embodiment of the compounds of formula ID, L³ is —C(O)—, L⁴ is —CH(CH₃)—, and Y is substituted or unsubstituted thienyl.

In another embodiment of the compounds of formula ID, L³ is —S(O)₂—, L⁴ is —CH(CH₃)—, and Y is substituted or unsubstituted thienyl.

In another embodiment of the compounds of formula ID, L³ is —C(R⁴)₂—, and Y is substituted or unsubstituted furanyl.

In another embodiment of the compounds of formula ID, L³ is —CH₂—, and Y is substituted or unsubstituted furanyl.

In another embodiment of the compounds of formula ID, L³ is —CH(CH₃)—, and Y is substituted or unsubstituted furanyl.

In another embodiment of the compounds of formula ID, L³ is —C(O)—, and Y is substituted or unsubstituted furanyl.

In another embodiment of the compounds of formula ID, L³ is —S(O)₂—, and Y is substituted or unsubstituted furanyl.

In another embodiment of the compounds of formula ID, L⁴ is a covalent bond, and Y is substituted or unsubstituted furanyl.

In another embodiment of the compounds of formula ID, L⁴ is —C(R⁴)₂—, and Y is substituted or unsubstituted furanyl.

In another embodiment of the compounds of formula ID, L⁴ is —CH₂—, and Y is substituted or unsubstituted furanyl.

In another embodiment of the compounds of formula ID, L⁴ is —CH(CH₃)—, and Y is substituted or unsubstituted furanyl.

In another embodiment of the compounds of formula ID, L³ is —C(R⁴)₂—, L⁴ is a covalent bond, and Y is substituted or unsubstituted furanyl.

In another embodiment of the compounds of formula ID, L³ is —CH₂—, L⁴ is a covalent bond, and Y is substituted or unsubstituted furanyl.

In another embodiment of the compounds of formula ID, L³ is —CH(CH₃)—, L⁴ is a covalent bond, and Y is substituted or unsubstituted furanyl.

In another embodiment of the compounds of formula ID, L³ is —C(O)—, L⁴ is a covalent bond, and Y is substituted or unsubstituted furanyl.

In another embodiment of the compounds of formula ID, L³ is —S(O)₂—, L⁴ is a covalent bond, and Y is substituted or unsubstituted furanyl.

In another embodiment of the compounds of formula ID, L³ is —C(R⁴)₂—, L⁴ is —C(R⁴)₂—, and Y is substituted or unsubstituted furanyl.

In another embodiment of the compounds of formula ID, L³ is —CH₂—, L⁴ is —C(R⁴)₂—, and Y is substituted or unsubstituted furanyl.

In another embodiment of the compounds of formula ID, L³ is —CH(CH₃)—, L⁴ is —C(R⁴)₂—, and Y is substituted or unsubstituted furanyl.

In another embodiment of the compounds of formula ID, L³ is —C(O)—, L⁴ is —C(R⁴)₂—, and Y is substituted or unsubstituted furanyl.

In another embodiment of the compounds of formula ID, L³ is —S(O)₂—, L⁴ is —C(R⁴)₂—, and Y is substituted or unsubstituted furanyl.

In another embodiment of the compounds of formula ID, L³ is —C(R⁴)₂—, L⁴ is —CH₂—, and Y is substituted or unsubstituted furanyl.

In another embodiment of the compounds of formula ID, L³ is —CH₂—, L⁴ is —CH₂—, and Y is substituted or unsubstituted furanyl.

In another embodiment of the compounds of formula ID, L³ is —CH(CH₃)—, L⁴ is —CH₂—, and Y is substituted or unsubstituted furanyl.

In another embodiment of the compounds of formula ID, L³ is —C(O)—, L⁴ is —CH₂—, and Y is substituted or unsubstituted furanyl.

In another embodiment of the compounds of formula ID, L³ is —S(O)₂—, L⁴ is —CH₂—, and Y is substituted or unsubstituted furanyl.

In another embodiment of the compounds of formula ID, L³ is —C(R⁴)₂—, L⁴ is —CH(CH₃)—, and Y is substituted or unsubstituted furanyl.

In another embodiment of the compounds of formula ID, L³ is —CH₂—, L⁴ is —CH(CH₃)—, and Y is substituted or unsubstituted furanyl.

In another embodiment of the compounds of formula ID, L³ is —CH(CH₃)—, L⁴ is —CH(CH₃)—, and Y is substituted or unsubstituted furanyl.

In another embodiment of the compounds of formula ID, L³ is —C(O)—, L⁴ is —CH(CH₃)—, and Y is substituted or unsubstituted furanyl.

In another embodiment of the compounds of formula ID, L³ is —S(O)₂—, L⁴ is —CH(CH₃)—, and Y is substituted or unsubstituted furanyl.

In another embodiment of the compounds of formula ID, L³ is —C(R⁴)₂—, and Y is substituted or unsubstituted purinyl.

In another embodiment of the compounds of formula ID, L³ is —CH₂—, and Y is substituted or unsubstituted purinyl.

In another embodiment of the compounds of formula ID, L³ is —CH(CH₃)—, and Y is substituted or unsubstituted purinyl.

In another embodiment of the compounds of formula ID, L³ is —C(O)—, and Y is substituted or unsubstituted purinyl.

In another embodiment of the compounds of formula ID, L³ is —S(O)₂—, and Y is substituted or unsubstituted purinyl.

In another embodiment of the compounds of formula ID, L⁴ is a covalent bond, and Y is substituted or unsubstituted purinyl.

In another embodiment of the compounds of formula ID, L⁴ is —C(R⁴)₂—, and Y is substituted or unsubstituted purinyl.

In another embodiment of the compounds of formula ID, L⁴ is —CH₂—, and Y is substituted or unsubstituted purinyl.

In another embodiment of the compounds of formula ID, L⁴ is —CH(CH₃)—, and Y is substituted or unsubstituted purinyl.

In another embodiment of the compounds of formula ID, L³ is —C(R⁴)₂—, L⁴ is a covalent bond, and Y is substituted or unsubstituted purinyl.

In another embodiment of the compounds of formula ID, L³ is —CH₂—, L⁴ is a covalent bond, and Y is substituted or unsubstituted purinyl.

In another embodiment of the compounds of formula ID, L³ is —CH(CH₃)—, L⁴ is a covalent bond, and Y is substituted or unsubstituted purinyl.

In another embodiment of the compounds of formula ID, L³ is —C(O)—, L⁴ is a covalent bond, and Y is substituted or unsubstituted purinyl.

In another embodiment of the compounds of formula ID, L³ is —S(O)₂—, L⁴ is a covalent bond, and Y is substituted or unsubstituted purinyl.

In another embodiment of the compounds of formula ID, L³ is —C(R⁴)₂—, L⁴ is —C(R⁴)₂—, and Y is substituted or unsubstituted purinyl.

In another embodiment of the compounds of formula ID, L³ is —CH₂—, L⁴ is —C(R⁴)₂—, and Y is substituted or unsubstituted purinyl.

In another embodiment of the compounds of formula ID, L³ is —CH(CH₃)—, L⁴ is —C(R⁴)₂—, and Y is substituted or unsubstituted purinyl.

In another embodiment of the compounds of formula ID, L³ is C(O)—, L⁴ is —C(R⁴)₂—, and Y is substituted or unsubstituted purinyl.

In another embodiment of the compounds of formula ID, L³ is —S(O)₂—, L⁴ is —C(R⁴)₂—, and Y is substituted or unsubstituted purinyl.

In another embodiment of the compounds of formula ID, L³ is —C(R⁴)₂—, L⁴ is —CH₂—, and Y is substituted or unsubstituted purinyl.

In another embodiment of the compounds of formula ID, L³ is —CH₂—, L⁴ is —CH₂—, and Y is substituted or unsubstituted purinyl.

In another embodiment of the compounds of formula ID, L³ is —CH(CH₃)—, L⁴ is —CH₂—, and Y is substituted or unsubstituted purinyl.

In another embodiment of the compounds of formula ID, L³ is —C(O)—, L⁴ is —CH₂—, and Y is substituted or unsubstituted purinyl.

In another embodiment of the compounds of formula ID, L³ is —S(O)₂—, L⁴ is —CH₂—, and Y is substituted or unsubstituted purinyl.

In another embodiment of the compounds of formula ID, L³ is —C(R⁴)₂—, L⁴ is —CH(CH₃)—, and Y is substituted or unsubstituted purinyl.

In another embodiment of the compounds of formula ID, L³ is —CH₂—, L⁴ is —CH(CH₃)—, and Y is substituted or unsubstituted purinyl.

In another embodiment of the compounds of formula ID, L³ is —CH(CH₃)—, L⁴ is —CH(CH₃)—, and Y is substituted or unsubstituted purinyl.

In another embodiment of the compounds of formula ID, L³ is —C(O)—, L⁴ is —CH(CH₃)—, and Y is substituted or unsubstituted purinyl.

In another embodiment of the compounds of formula ID, L³ is —S(O)₂—, L⁴ is —CH(CH₃)—, and Y is substituted or unsubstituted purinyl.

In another embodiment of the compounds of formula ID, L³ is —C(R⁴)₂—, and Y is substituted or unsubstituted benzimidazolyl.

In another embodiment of the compounds of formula ID, L³ is —CH₂—, and Y is substituted or unsubstituted benzimidazolyl.

In another embodiment of the compounds of formula ID, L³ is —CH(CH₃)—, and Y is substituted or unsubstituted benzimidazolyl.

In another embodiment of the compounds of formula ID, L³ is —C(O)—, and Y is substituted or unsubstituted benzimidazolyl.

In another embodiment of the compounds of formula ID, L³ is —S(O)₂—, and Y is substituted or unsubstituted benzimidazolyl.

In another embodiment of the compounds of formula ID, L⁴ is a covalent bond, and Y is substituted or unsubstituted benzimidazolyl.

In another embodiment of the compounds of formula ID, L⁴ is —C(R⁴)₂—, and Y is substituted or unsubstituted benzimidazolyl.

In another embodiment of the compounds of formula ID, L⁴ is —CH₂—, and Y is substituted or unsubstituted benzimidazolyl.

In another embodiment of the compounds of formula ID, L⁴ is —CH(CH₃)—, and Y is substituted or unsubstituted benzimidazolyl.

In another embodiment of the compounds of formula ID, L³ is —C(R⁴)₂—, L⁴ is a covalent bond, and Y is substituted or unsubstituted benzimidazolyl.

In another embodiment of the compounds of formula ID, L³ is —CH₂—, L⁴ is a covalent bond, and Y is substituted or unsubstituted benzimidazolyl.

In another embodiment of the compounds of formula ID, L³ is —CH(CH₃)—, L⁴ is a covalent bond, and Y is substituted or unsubstituted benzimidazolyl.

In another embodiment of the compounds of formula ID, L³ is —C(O)—, L⁴ is a covalent bond, and Y is substituted or unsubstituted benzimidazolyl.

In another embodiment of the compounds of formula ID, L³ is —S(O)₂—, L⁴ is a covalent bond, and Y is substituted or unsubstituted benzimidazolyl.

In another embodiment of the compounds of formula ID, L³ is —C(R⁴)₂—, L⁴ is —C(R⁴)₂—, and Y is substituted or unsubstituted benzimidazolyl.

In another embodiment of the compounds of formula ID, L³ is —CH₂—, L⁴ is —C(R⁴)₂—, and Y is substituted or unsubstituted benzimidazolyl.

In another embodiment of the compounds of formula ID, L³ is —CH(CH₃)—, L⁴ is —C(R⁴)₂—, and Y is substituted or unsubstituted benzimidazolyl.

In another embodiment of the compounds of formula ID, L³ is —C(O)—, L⁴ is —C(R⁴)₂—, and Y is substituted or unsubstituted benzimidazolyl.

In another embodiment of the compounds of formula ID, L³ is —S(O)₂—, L⁴ is —C(R⁴)₂—, and Y is substituted or unsubstituted benzimidazolyl.

In another embodiment of the compounds of formula ID, L³ is —C(R⁴)₂—, L⁴ is —CH₂—, and Y is substituted or unsubstituted benzimidazolyl.

In another embodiment of the compounds of formula ID, L³ is —CH₂—, L⁴ is —CH₂—, and Y is substituted or unsubstituted benzimidazolyl.

In another embodiment of the compounds of formula ID, L³ is —CH(CH₃)—, L⁴ is —CH₂—, and Y is substituted or unsubstituted benzimidazolyl.

In another embodiment of the compounds of formula ID, L³ is —C(O)—, L⁴ is —CH₂—, and Y is substituted or unsubstituted benzimidazolyl.

In another embodiment of the compounds of formula ID, L³ is —S(O)₂—, L⁴ is —CH₂—, and Y is substituted or unsubstituted benzimidazolyl.

In another embodiment of the compounds of formula ID, L³ is —C(R⁴)₂—, L⁴ is —CH(CH₃)—, and Y is substituted or unsubstituted benzimidazolyl.

In another embodiment of the compounds of formula ID, L³ is —CH₂—, L⁴ is —CH(CH₃)—, and Y is substituted or unsubstituted benzimidazolyl.

In another embodiment of the compounds of formula ID, L³ is —CH(CH₃)—, L⁴ is —CH(CH₃)—, and Y is substituted or unsubstituted benzimidazolyl.

In another embodiment of the compounds of formula ID, L³ is —C(O)—, L⁴ is —CH(CH₃)—, and Y is substituted or unsubstituted benzimidazolyl.

In another embodiment of the compounds of formula ID, L³ is —S(O)₂—, L⁴ is —CH(CH₃)—, and Y is substituted or unsubstituted benzimidazolyl.

In another embodiment of the compounds of formula ID, L³ is —C(R⁴)₂—, and Y is substituted or unsubstituted pyrrolyl.

In another embodiment of the compounds of formula ID, L³ is —CH₂—, and Y is substituted or unsubstituted pyrrolyl.

In another embodiment of the compounds of formula ID, L³ is —CH(CH₃)—, and Y is substituted or unsubstituted pyrrolyl.

In another embodiment of the compounds of formula ID, L³ is —C(O)—, and Y is substituted or unsubstituted pyrrolyl.

In another embodiment of the compounds of formula ID, L³ is —S(O)₂—, and Y is substituted or unsubstituted pyrrolyl.

In another embodiment of the compounds of formula ID, L⁴ is a covalent bond, and Y is substituted or unsubstituted pyrrolyl.

In another embodiment of the compounds of formula ID, L⁴ is —C(R⁴)₂—, and Y is substituted or unsubstituted pyrrolyl.

In another embodiment of the compounds of formula ID, L⁴ is —CH₂—, and Y is substituted or unsubstituted pyrrolyl.

In another embodiment of the compounds of formula ID, L⁴ is —CH(CH₃)—, and Y is substituted or unsubstituted pyrrolyl.

In another embodiment of the compounds of formula ID, L³ is —C(R⁴)₂—, L⁴ is a covalent bond, and Y is substituted or unsubstituted pyrrolyl.

In another embodiment of the compounds of formula ID, L³ is —CH₂—, L⁴ is a covalent bond, and Y is substituted or unsubstituted pyrrolyl.

In another embodiment of the compounds of formula ID, L³ is —CH(CH₃)—, L⁴ is a covalent bond, and Y is substituted or unsubstituted pyrrolyl.

In another embodiment of the compounds of formula ID, L³ is —C(O)—, L⁴ is a covalent bond, and Y is substituted or unsubstituted pyrrolyl.

In another embodiment of the compounds of formula ID, L³ is —S(O)₂—, L⁴ is a covalent bond, and Y is substituted or unsubstituted pyrrolyl.

In another embodiment of the compounds of formula ID, L³ is —C(R⁴)₂—, L⁴ is —C(R⁴)₂—, and Y is substituted or unsubstituted pyrrolyl.

In another embodiment of the compounds of formula ID, L³ is —CH₂—, L⁴ is —C(R⁴)₂—, and Y is substituted or unsubstituted pyrrolyl.

In another embodiment of the compounds of formula ID, L³ is —CH(CH₃)—, L⁴ is —C(R⁴)₂—, and Y is substituted or unsubstituted pyrrolyl.

In another embodiment of the compounds of formula ID, L³ is —C(O)—, L⁴ is —C(R⁴)₂—, and Y is substituted or unsubstituted pyrrolyl.

In another embodiment of the compounds of formula ID, L³ is —S(O)₂—, L⁴ is —C(R⁴)₂—, and Y is substituted or unsubstituted pyrrolyl.

In another embodiment of the compounds of formula ID, L³ is —C(R⁴)₂—, L⁴ is —CH₂—, and Y is substituted or unsubstituted pyrrolyl.

In another embodiment of the compounds of formula ID, L³ is —CH₂—, L⁴ is —CH₂—, and Y is substituted or unsubstituted pyrrolyl.

In another embodiment of the compounds of formula ID, L³ is —CH(CH₃)—, L⁴ is —CH₂—, and Y is substituted or unsubstituted pyrrolyl.

In another embodiment of the compounds of formula ID, L³ is —C(O)—, L⁴ is —CH₂—, and Y is substituted or unsubstituted pyrrolyl.

In another embodiment of the compounds of formula ID, L³ is —S(O)₂—, L⁴ is —CH₂—, and Y is substituted or unsubstituted pyrrolyl.

In another embodiment of the compounds of formula ID, L³ is —C(R⁴)₂—, L⁴ is —CH(CH₃)—, and Y is substituted or unsubstituted pyrrolyl.

In another embodiment of the compounds of formula ID, L³ is —CH₂—, L⁴ is —CH(CH₃)—, and Y is substituted or unsubstituted pyrrolyl.

In another embodiment of the compounds of formula ID, L³ is —CH(CH₃)—, L⁴ is —CH(CH₃)—, and Y is substituted or unsubstituted pyrrolyl.

In another embodiment of the compounds of formula ID, L³ is —C(O)—, L⁴ is —CH(CH₃)—, and Y is substituted or unsubstituted pyrrolyl.

In another embodiment of the compounds of formula ID, L³ is —S(O)₂—, L⁴ is —CH(CH₃)—, and Y is substituted or unsubstituted pyrrolyl.

In another embodiment of the compounds of formula ID, L³ is —C(R⁴)₂—, and Y is substituted or unsubstituted quinolyl.

In another embodiment of the compounds of formula ID, L³ is —CH₂—, and Y is substituted or unsubstituted quinolyl.

In another embodiment of the compounds of formula ID, L³ is —CH(CH₃)—, and Y is substituted or unsubstituted quinolyl.

In another embodiment of the compounds of formula ID, L³ is —C(O)—, and Y is substituted or unsubstituted quinolyl.

In another embodiment of the compounds of formula ID, L³ is —S(O)₂—, and Y is substituted or unsubstituted quinolyl.

In another embodiment of the compounds of formula ID, L⁴ is a covalent bond, and Y is substituted or unsubstituted quinolyl.

In another embodiment of the compounds of formula ID, L⁴ is —C(R⁴)₂—, and Y is substituted or unsubstituted quinolyl.

In another embodiment of the compounds of formula ID, L⁴ is —CH₂—, and Y is substituted or unsubstituted quinolyl.

In another embodiment of the compounds of formula ID, L⁴ is —CH(CH₃)—, and Y is substituted or unsubstituted quinolyl.

In another embodiment of the compounds of formula ID, L³ is —C(R⁴)₂—, L⁴ is a covalent bond, and Y is substituted or unsubstituted quinolyl.

In another embodiment of the compounds of formula ID, L³ is —CH₂—, L⁴ is a covalent bond, and Y is substituted or unsubstituted quinolyl.

In another embodiment of the compounds of formula ID, L³ is —CH(CH₃)—, L⁴ is a covalent bond, and Y is substituted or unsubstituted quinolyl.

In another embodiment of the compounds of formula ID, L³ is —C(O)—, L⁴ is a covalent bond, and Y is substituted or unsubstituted quinolyl.

In another embodiment of the compounds of formula ID, L³ is —S(O)₂—, L⁴ is a covalent bond, and Y is substituted or unsubstituted quinolyl.

In another embodiment of the compounds of formula ID, L³ is —C(R⁴)₂—, L⁴ is —C(R⁴)₂—, and Y is substituted or unsubstituted quinolyl.

In another embodiment of the compounds of formula ID, L³ is —CH₂—, L⁴ is —C(R⁴)₂—, and Y is substituted or unsubstituted quinolyl.

In another embodiment of the compounds of formula ID, L³ is —CH(CH₃)—, L⁴ is —C(R⁴)₂—, and Y is substituted or unsubstituted quinolyl.

In another embodiment of the compounds of formula ID, L³ is —C(O)—, L⁴ is —C(R⁴)₂—, and Y is substituted or unsubstituted quinolyl.

In another embodiment of the compounds of formula ID, L³ is —S(O)₂—, L⁴ is —C(R⁴)₂—, and Y is substituted or unsubstituted quinolyl.

In another embodiment of the compounds of formula ID, L³ is —C(R⁴)₂—, L⁴ is —CH₂—, and Y is substituted or unsubstituted quinolyl.

In another embodiment of the compounds of formula ID, L³ is —CH₂—, L⁴ is —CH₂—, and Y is substituted or unsubstituted quinolyl.

In another embodiment of the compounds of formula ID, L³ is —CH(CH₃)—, L⁴ is —CH₂—, and Y is substituted or unsubstituted quinolyl.

In another embodiment of the compounds of formula ID, L³ is —C(O)—, L⁴ is —CH₂—, and Y is substituted or unsubstituted quinolyl.

In another embodiment of the compounds of formula ID, L³ is —S(O)₂—, L⁴ is —CH₂—, and Y is substituted or unsubstituted quinolyl.

In another embodiment of the compounds of formula ID, L³ is —C(R⁴)₂—, L⁴ is —CH(CH₃)—, and Y is substituted or unsubstituted quinolyl.

In another embodiment of the compounds of formula ID, L³ is —CH₂—, L⁴ is —CH(CH₃)—, and Y is substituted or unsubstituted quinolyl.

In another embodiment of the compounds of formula ID, L³ is —CH(CH₃)—, L⁴ is —CH(CH₃)—, and Y is substituted or unsubstituted quinolyl.

In another embodiment of the compounds of formula ID, L³ is —C(O)—, L⁴ is —CH(CH₃)—, and Y is substituted or unsubstituted quinolyl.

In another embodiment of the compounds of formula ID, L³ is —S(O)₂—, L⁴ is —CH(CH₃)—, and Y is substituted or unsubstituted quinolyl.

In another embodiment of the compounds of formula ID, R⁶ and -L⁴-Y, together with the nitrogen atom to which they are shown attached, form a substituted or unsubstituted heterocyclyl.

In another embodiment of the compounds of formula ID, R⁶ and -L⁴-Y, together with the nitrogen atom to which they are shown attached, form a substituted or unsubstituted piperazine.

In another embodiment of the compounds of formula ID, R⁶ and -L⁴-Y, together with the nitrogen atom to which they are shown attached, form a substituted or unsubstituted piperidine.

In another embodiment of the compounds of formula ID, R⁶ and -L⁴-Y, together with the nitrogen atom to which they are shown attached, form a substituted or unsubstituted pyrrolidinyl.

In another embodiment of the compounds of formula ID, R⁶ and -L⁴-Y, together with the nitrogen atom to which they are shown attached, form a substituted or unsubstituted morpholinyl.

In another embodiment of the compounds of formula ID, R⁶ and -L⁴-Y, together with the nitrogen atom to which they are shown attached, form a substituted or unsubstituted tetraazacyclotetradecanyl.

In another embodiment of the compounds of formula ID, R⁶ and -L⁴-Y, together with the nitrogen atom to which they are shown attached, form a substituted or unsubstituted diazabicycloheptanyl.

In another embodiment of the compounds of formula ID, L³ is —C(R⁴)₂—, and R⁶ and -L⁴-Y, together with the nitrogen atom to which they are shown attached, form a substituted or unsubstituted heterocyclyl.

In another embodiment of the compounds of formula ID, L³ is —CH₂—, and R⁶ and -L⁴-Y, together with the nitrogen atom to which they are shown attached, form a substituted or unsubstituted heterocyclyl.

In another embodiment of the compounds of formula ID, L³ is —CH(CH₃)—, and R⁶ and -L⁴-Y, together with the nitrogen atom to which they are shown attached, form a substituted or unsubstituted heterocyclyl.

In another embodiment of the compounds of formula ID, L³ is —C(O)—, and R⁶ and -L⁴-Y, together with the nitrogen atom to which they are shown attached, form a substituted or unsubstituted heterocyclyl.

In another embodiment of the compounds of formula ID, L³ is —S(O)₂—, and R⁶ and -L⁴-Y, together with the nitrogen atom to which they are shown attached, form a substituted or unsubstituted heterocyclyl.

In another embodiment of the compounds of formula ID, L³ is —C(R⁴)₂—, and R⁶ and -L⁴-Y, together with the nitrogen atom to which they are shown attached, form a substituted or unsubstituted piperidinyl.

In another embodiment of the compounds of formula ID, L³ is —CH₂—, and R⁶ and -L⁴-Y, together with the nitrogen atom to which they are shown attached, form a substituted or unsubstituted piperidinyl.

In another embodiment of the compounds of formula ID, L³ is —CH(CH₃)—, and R⁶ and -L⁴-Y, together with the nitrogen atom to which they are shown attached, form a substituted or unsubstituted piperidinyl.

In another embodiment of the compounds of formula ID, L³ is —C(O)—, and R⁶ and -L⁴-Y, together with the nitrogen atom to which they are shown attached, form a substituted or unsubstituted piperidinyl.

In another embodiment of the compounds of formula ID, L³ is —S(O)₂—, and R⁶ and -L⁴-Y, together with the nitrogen atom to which they are shown attached, form a substituted or unsubstituted piperidinyl.

In another embodiment of the compounds of formula ID, L³ is —C(R⁴)₂—, and R⁶ and -L⁴-Y, together with the nitrogen atom to which they are shown attached, form a substituted or unsubstituted piperazinyl.

In another embodiment of the compounds of formula ID, L³ is —CH₂—, and R⁶ and -L⁴-Y, together with the nitrogen atom to which they are shown attached, form a substituted or unsubstituted piperazinyl.

In another embodiment of the compounds of formula ID, L³ is —CH(CH₃)—, and R⁶ and -L⁴-Y, together with the nitrogen atom to which they are shown attached, form a substituted or unsubstituted piperazinyl.

In another embodiment of the compounds of formula ID, L³ is —C(O)—, and R⁶ and -L⁴-Y, together with the nitrogen atom to which they are shown attached, form a substituted or unsubstituted piperazinyl.

In another embodiment of the compounds of formula ID, L³ is —S(O)₂—, and R⁶ and -L⁴-Y, together with the nitrogen atom to which they are shown attached, form a substituted or unsubstituted piperazinyl.

In another embodiment of the compounds of formula ID, L³ is —C(R⁴)₂—, and R⁶ and -L⁴-Y, together with the nitrogen atom to which they are shown attached, form a substituted or unsubstituted pyrrolidinyl.

In another embodiment of the compounds of formula ID, L³ is —CH₂—, and R⁶ and -L⁴-Y, together with the nitrogen atom to which they are shown attached, form a substituted or unsubstituted pyrrolidinyl.

In another embodiment of the compounds of formula ID, L³ is —CH(CH₃)—, and R⁶ and -L⁴-Y, together with the nitrogen atom to which they are shown attached, form a substituted or unsubstituted pyrrolidinyl.

In another embodiment of the compounds of formula ID, L³ is —C(O)—, and R⁶ and -L⁴-Y, together with the nitrogen atom to which they are shown attached, form a substituted or unsubstituted pyrrolidinyl.

In another embodiment of the compounds of formula ID, L³ is —S(O)₂—, and R⁶ and -L⁴-Y, together with the nitrogen atom to which they are shown attached, form a substituted or unsubstituted pyrrolidinyl.

In another embodiment of the compounds of formula ID, L³ is —C(R⁴)₂—, and R⁶ and -L⁴-Y, together with the nitrogen atom to which they are shown attached, form a substituted or unsubstituted morpholinyl.

In another embodiment of the compounds of formula ID, L³ is —CH₂—, and R⁶ and -L⁴-Y, together with the nitrogen atom to which they are shown attached, form a substituted or unsubstituted morpholinyl.

In another embodiment of the compounds of formula ID, L³ is —CH(CH₃)—, and R⁶ and -L⁴-Y, together with the nitrogen atom to which they are shown attached, form a substituted or unsubstituted morpholinyl.

In another embodiment of the compounds of formula ID, L³ is —C(O)—, and R⁶ and -L⁴-Y, together with the nitrogen atom to which they are shown attached, form a substituted or unsubstituted morpholinyl.

In another embodiment of the compounds of formula ID, L³ is —S(O)₂—, and R⁶ and -L⁴-Y, together with the nitrogen atom to which they are shown attached, form a substituted or unsubstituted morpholinyl.

In another embodiment of the compounds of formula ID, L³ is —C(R⁴)₂—, and R⁶ and -L⁴-Y, together with the nitrogen atom to which they are shown attached, form a substituted or unsubstituted tetraazacyclotetradecanyl.

In another embodiment of the compounds of formula ID, L³ is —CH₂—, and R⁶ and -L⁴-Y, together with the nitrogen atom to which they are shown attached, form a substituted or unsubstituted tetraazacyclotetradecanyl.

In another embodiment of the compounds of formula ID, L³ is —CH(CH₃)—, and R⁶ and -L⁴-Y, together with the nitrogen atom to which they are shown attached, form a substituted or unsubstituted tetraazacyclotetradecanyl.

In another embodiment of the compounds of formula ID, L³ is —C(O)—, and R⁶ and -L⁴-Y, together with the nitrogen atom to which they are shown attached, form a substituted or unsubstituted tetraazacyclotetradecanyl.

In another embodiment of the compounds of formula ID, L³ is —S(O)₂—, and R⁶ and -L⁴-Y, together with the nitrogen atom to which they are shown attached, form a substituted or unsubstituted tetraazacyclotetradecanyl.

In another embodiment of the compounds of formula ID, L³ is —C(R⁴)₂—, and R⁶ and -L⁴-Y, together with the nitrogen atom to which they are shown attached, form a substituted or unsubstituted diazabicycloheptanyl.

In another embodiment of the compounds of formula ID, L³ is —CH₂—, and R⁶ and -L⁴-Y, together with the nitrogen atom to which they are shown attached, form a substituted or unsubstituted diazabicycloheptanyl.

In another embodiment of the compounds of formula ID, L³ is —CH(CH₃)—, and R⁶ and -L⁴-Y, together with the nitrogen atom to which they are shown attached, form a substituted or unsubstituted diazabicycloheptanyl.

In another embodiment of the compounds of formula ID, L³ is —C(O)—, and R⁶ and -L⁴-Y, together with the nitrogen atom to which they are shown attached, form a substituted or unsubstituted diazabicycloheptanyl.

In another embodiment of the compounds of formula ID, L³ is —S(O)₂—, and R⁶ and -L⁴-Y, together with the nitrogen atom to which they are shown attached, form a substituted or unsubstituted diazabicycloheptanyl.

In another embodiment of the compounds of formula ID, L³ is —C(R⁴)₂—, and Y is alkoxyalkyl.

In another embodiment of the compounds of formula ID, L³ is —CH₂—, and Y is alkoxyalkyl.

In another embodiment of the compounds of formula ID, L³ is —CH(CH₃)—, and Y is alkoxyalkyl.

In another embodiment of the compounds of formula ID, L³ is —C(O)—, and Y is alkoxyalkyl.

In another embodiment of the compounds of formula ID, L³ is —S(O)₂—, and Y is alkoxyalkyl.

In another embodiment of the compounds of formula ID, L³ is —C(R⁴)₂—, and Y is methoxyalkyl.

In another embodiment of the compounds of formula ID, L³ is —CH₂—, and Y is methoxyalkyl.

In another embodiment of the compounds of formula ID, L³ is —CH(CH₃)—, and Y is methoxyalkyl.

In another embodiment of the compounds of formula ID, L³ is —C(O)—, and Y is methoxyalkyl.

In another embodiment of the compounds of formula ID, L³ is —S(O)₂—, and Y is methoxyalkyl.

In another embodiment of the compounds of formula ID, L³ is —C(R⁴)₂—, L⁴ is a covalent bond, and Y is alkoxyalkyl.

In another embodiment of the compounds of formula ID, L³ is —CH₂—, L⁴ is a covalent bond, and Y is alkoxyalkyl.

In another embodiment of the compounds of formula ID, L³ is —CH(CH₃)—, L⁴ is a covalent bond, and Y is alkoxyalkyl.

In another embodiment of the compounds of formula ID, L³ is —C(O)—, L⁴ is a covalent bond, and Y is alkoxyalkyl.

In another embodiment of the compounds of formula ID, L³ is —S(O)₂—, L⁴ is a covalent bond, and Y is alkoxyalkyl.

In another embodiment of the compounds of formula ID, L³ is —C(R⁴)₂—, L⁴ is —C(R⁴)₂—, and Y is alkoxyalkyl.

In another embodiment of the compounds of formula ID, L³ is —CH₂—, L⁴ is —C(R⁴)₂—, and Y is alkoxyalkyl.

In another embodiment of the compounds of formula ID, L³ is —CH(CH₃)—, L⁴ is —C(R⁴)₂—, and Y is alkoxyalkyl.

In another embodiment of the compounds of formula ID, L³ is —C(O)—, L⁴ is —C(R⁴)₂—, and Y is alkoxyalkyl.

In another embodiment of the compounds of formula ID, L³ is —S(O)₂—, L⁴ is —C(R⁴)₂—, and Y is alkoxyalkyl.

In another embodiment of the compounds of formula ID, L³ is —C(R⁴)₂—, L⁴ is —CH₂—, and Y is alkoxyalkyl.

In another embodiment of the compounds of formula ID, L³ is —CH₂—, L⁴ is —CH₂—, and Y is alkoxyalkyl.

In another embodiment of the compounds of formula ID, L³ is —CH(CH₃)—, L⁴ is —CH₂—, and Y is alkoxyalkyl.

In another embodiment of the compounds of formula ID, L³ is —C(O)—, L⁴ is —CH₂—, and Y is alkoxyalkyl.

In another embodiment of the compounds of formula ID, L³ is —S(O)₂—, L⁴ is —CH₂—, and Y is alkoxyalkyl.

In another embodiment of the compounds of formula ID, L³ is —C(R⁴)₂—, L⁴ is —CH(CH₃)—, and Y is alkoxyalkyl.

In another embodiment of the compounds of formula ID, L³ is —CH₂—, L⁴ is —CH(CH₃)—, and Y is alkoxyalkyl.

In another embodiment of the compounds of formula ID, L³ is —CH(CH₃)—, L⁴ is —CH(CH₃)—, and Y is alkoxyalkyl.

In another embodiment of the compounds of formula ID, L³ is —C(O)—, L⁴ is —CH(CH₃)—, and Y is alkoxyalkyl.

In another embodiment of the compounds of formula ID, L³ is —S(O)₂—, L⁴ is —CH(CH₃)—, and Y is alkoxyalkyl.

In another embodiment of the compounds of formula ID, L³ is —C(R⁴)₂—, L⁴ is a covalent bond, and Y is methoxyalkyl.

In another embodiment of the compounds of formula ID, L³ is —CH₂—, L⁴ is a covalent bond, and Y is methoxyalkyl.

In another embodiment of the compounds of formula ID, L³ is —CH(CH₃)—, L⁴ is a covalent bond, and Y is methoxyalkyl.

In another embodiment of the compounds of formula ID, L³ is —C(O)—, L⁴ is a covalent bond, and Y is methoxyalkyl.

In another embodiment of the compounds of formula ID, L³ is —S(O)₂—, L⁴ is a covalent bond, and Y is methoxyalkyl.

In another embodiment of the compounds of formula ID, L³ is —C(R⁴)₂—, L⁴ is —C(R⁴)₂—, and Y is methoxyalkyl.

In another embodiment of the compounds of formula ID, L³ is —CH₂—, L⁴ is —C(R⁴)₂—, and Y is methoxyalkyl.

In another embodiment of the compounds of formula ID, L³ is —CH(CH₃)—, L⁴ is —C(R⁴)₂—, and Y is methoxyalkyl.

In another embodiment of the compounds of formula ID, L³ is —C(O)—, L⁴ is —C(R⁴)₂—, and Y is methoxyalkyl.

In another embodiment of the compounds of formula ID, L³ is —S(O)₂—, L⁴ is —C(R⁴)₂—, and Y is methoxyalkyl.

In another embodiment of the compounds of formula ID, L³ is —C(R⁴)₂—, L⁴ is —CH₂—, and Y is methoxyalkyl.

In another embodiment of the compounds of formula ID, L³ is —CH₂—, L⁴ is —CH₂—, and Y is methoxyalkyl.

In another embodiment of the compounds of formula ID, L³ is —CH(CH₃)—, L⁴ is —CH₂—, and Y is methoxyalkyl.

In another embodiment of the compounds of formula ID, L³ is —C(O)—, L⁴ is —CH₂—, and Y is methoxyalkyl.

In another embodiment of the compounds of formula ID, L³ is —S(O)₂—, L⁴ is —CH₂—, and Y is methoxyalkyl.

In another embodiment of the compounds of formula ID, L³ is —C(R⁴)₂—, L⁴ is —CH(CH₃)—, and Y is methoxyalkyl.

In another embodiment of the compounds of formula ID, L³ is —CH₂—, L⁴ is —CH(CH₃)—, and Y is methoxyalkyl.

In another embodiment of the compounds of formula ID, L³ is —CH(CH₃)—, L⁴ is —CH(CH₃)—, and Y is methoxyalkyl.

In another embodiment of the compounds of formula ID, L³ is —C(O)—, L⁴ is —CH(CH₃)—, and Y is methoxyalkyl.

In another embodiment of the compounds of formula ID, L³ is —S(O)₂—, L⁴ is —CH(CH₃)—, and Y is methoxyalkyl.

In another embodiment of the compounds of formula ID, L³ is —C(R⁴)₂—, L⁴ is a covalent bond, R⁶ is H, and Y is alkoxyalkyl.

In another embodiment of the compounds of formula ID, L³ is —CH₂—, L⁴ is a covalent bond, R⁶ is H, and Y is alkoxyalkyl.

In another embodiment of the compounds of formula ID, L³ is —CH(CH₃)—, L⁴ is a covalent bond, R⁶ is H, and Y is alkoxyalkyl.

In another embodiment of the compounds of formula ID, L³ is —C(O)—, L⁴ is a covalent bond, R⁶ is H, and Y is alkoxyalkyl.

In another embodiment of the compounds of formula ID, L³ is —S(O)₂—, L⁴ is a covalent bond, R⁶ is H, and Y is alkoxyalkyl.

In another embodiment of the compounds of formula ID, L³ is —C(R⁴)₂—, L⁴ is —C(R⁴)₂—, R⁶ is H, and Y is alkoxyalkyl.

In another embodiment of the compounds of formula ID, L³ is —CH₂—, L⁴ is —C(R⁴)₂—, R⁶ is H, and Y is alkoxyalkyl.

In another embodiment of the compounds of formula ID, L³ is —CH(CH₃)—, L⁴ is —C(R⁴)₂—, R⁶ is H, and Y is alkoxyalkyl.

In another embodiment of the compounds of formula ID, L³ is —C(O)—, L⁴ is —C(R⁴)₂—, R⁶ is H, and Y is alkoxyalkyl.

In another embodiment of the compounds of formula ID, L³ is —S(O)₂—, L⁴ is —C(R⁴)₂—, R⁶ is H, and Y is alkoxyalkyl.

In another embodiment of the compounds of formula ID, L³ is —C(R⁴)₂—, L⁴ is —CH₂—, R⁶ is H, and Y is alkoxyalkyl.

In another embodiment of the compounds of formula ID, L³ is —CH₂—, L⁴ is —CH₂—, R⁶ is H, and Y is alkoxyalkyl.

In another embodiment of the compounds of formula ID, L³ is —CH(CH₃)—, L⁴ is —CH₂—, R⁶ is H, and Y is alkoxyalkyl.

In another embodiment of the compounds of formula ID, L³ is —C(O)—, L⁴ is —CH₂—, R⁶ is H, and Y is alkoxyalkyl.

In another embodiment of the compounds of formula ID, L³ is —S(O)₂—, L⁴ is —CH₂—, R⁶ is H, and Y is alkoxyalkyl.

In another embodiment of the compounds of formula ID, L³ is —C(R⁴)₂—, L⁴ is —CH(CH₃)—, R⁶ is H, and Y is alkoxyalkyl.

In another embodiment of the compounds of formula ID, L³ is —CH₂—, L⁴ is —CH(CH₃)—, R⁶ is H, and Y is alkoxyalkyl.

In another embodiment of the compounds of formula ID, L³ is —CH(CH₃)—, L⁴ is —CH(CH₃)—, R⁶ is H, and Y is alkoxyalkyl.

In another embodiment of the compounds of formula ID, L³ is —C(O)—, L⁴ is —CH(CH₃)—, R⁶ is H, and Y is alkoxyalkyl.

In another embodiment of the compounds of formula ID, L³ is —S(O)₂—, L⁴ is —CH(CH₃)—, R⁶ is H, and Y is alkoxyalkyl.

In another embodiment of the compounds of formula ID, L³ is —C(R⁴)₂—, L⁴ is a covalent bond, R⁶ is alkyl, and Y is alkoxyalkyl.

In another embodiment of the compounds of formula ID, L³ is —CH₂—, L⁴ is a covalent bond, R⁶ is alkyl, and Y is alkoxyalkyl.

In another embodiment of the compounds of formula ID, L³ is —CH(CH₃)—, L⁴ is a covalent bond, R⁶ is alkyl, and Y is alkoxyalkyl.

In another embodiment of the compounds of formula ID, L³ is —C(O)—, L⁴ is a covalent bond, R⁶ is alkyl, and Y is alkoxyalkyl.

In another embodiment of the compounds of formula ID, L³ is —S(O)₂—, L⁴ is a covalent bond, R⁶ is alkyl, and Y is alkoxyalkyl.

In another embodiment of the compounds of formula ID, L³ is —C(R⁴)₂—, L⁴ is —C(R⁴)₂—, R⁶ is alkyl, and Y is alkoxyalkyl.

In another embodiment of the compounds of formula ID, L³ is —CH₂—, L⁴ is —C(R⁴)₂—, R⁶ is alkyl, and Y is alkoxyalkyl.

In another embodiment of the compounds of formula ID, L³ is —CH(CH₃)—, L⁴ is —C(R⁴)₂—, R⁶ is alkyl, and Y is alkoxyalkyl.

In another embodiment of the compounds of formula ID, L³ is —C(O)—, L⁴ is —C(R⁴)₂—, R⁶ is alkyl, and Y is alkoxyalkyl.

In another embodiment of the compounds of formula ID, L³ is —S(O)₂—, L⁴ is —C(R⁴)₂—, R⁶ is alkyl, and Y is alkoxyalkyl.

In another embodiment of the compounds of formula ID, L³ is —C(R⁴)₂—, L⁴ is —CH₂—, R⁶ is alkyl, and Y is alkoxyalkyl.

In another embodiment of the compounds of formula ID, L³ is —CH₂—, L⁴ is —CH₂—, R⁶ is alkyl, and Y is alkoxyalkyl.

In another embodiment of the compounds of formula ID, L³ is —CH(CH₃)—, L⁴ is —CH₂—, R⁶ is alkyl, and Y is alkoxyalkyl.

In another embodiment of the compounds of formula ID, L³ is —C(O)—, L⁴ is —CH₂—, R⁶ is alkyl, and Y is alkoxyalkyl.

In another embodiment of the compounds of formula ID, L³ is —S(O)₂—, L⁴ is —CH₂—, R⁶ is alkyl, and Y is alkoxyalkyl.

In another embodiment of the compounds of formula ID, L³ is —C(R⁴)₂—, L⁴ is —CH(CH₃)—, R⁶ is alkyl, and Y is alkoxyalkyl.

In another embodiment of the compounds of formula ID, L³ is —CH₂—, L⁴ is —CH(CH₃)—, R⁶ is alkyl, and Y is alkoxyalkyl.

In another embodiment of the compounds of formula ID, L³ is —CH(CH₃)—, L⁴ is —CH(CH₃)—, R⁶ is alkyl, and Y is alkoxyalkyl.

In another embodiment of the compounds of formula ID, L³ is —C(O)—, L⁴ is —CH(CH₃)—, R⁶ is alkyl, and Y is alkoxyalkyl.

In another embodiment of the compounds of formula ID, L³ is —S(O)₂—, L⁴ is —CH(CH₃)—, R⁶ is alkyl, and Y is alkoxyalkyl.

In another embodiment of the compounds of formula ID, L³ is —C(R⁴)₂—, L⁴ is a covalent bond, R⁶ is alkoxyalkyl, and Y is alkoxyalkyl.

In another embodiment of the compounds of formula ID, L³ is —CH₂—, L⁴ is a covalent bond, R⁶ is alkoxyalkyl, and Y is alkoxyalkyl.

In another embodiment of the compounds of formula ID, L³ is —CH(CH₃)—, L⁴ is a covalent bond, R⁶ is alkoxyalkyl, and Y is alkoxyalkyl.

In another embodiment of the compounds of formula ID, L³ is —C(O)—, L⁴ is a covalent bond, R⁶ is alkoxyalkyl, and Y is alkoxyalkyl.

In another embodiment of the compounds of formula ID, L³ is —S(O)₂—, L⁴ is a covalent bond, R⁶ is alkoxyalkyl, and Y is alkoxyalkyl.

In another embodiment of the compounds of formula ID, L³ is —C(R⁴)₂—, L⁴ is —C(R⁴)₂—, R⁶ is alkoxyalkyl, and Y is alkoxyalkyl.

In another embodiment of the compounds of formula ID, L³ is —CH₂—, L⁴ is —C(R⁴)₂—, R⁶ is alkoxyalkyl, and Y is alkoxyalkyl.

In another embodiment of the compounds of formula ID, L³ is —CH(CH₃)—, L⁴ is —C(R⁴)₂—, R⁶ is alkoxyalkyl, and Y is alkoxyalkyl.

In another embodiment of the compounds of formula ID, L³ is —C(O)—, L⁴ is —C(R⁴)₂—, R⁶ is alkoxyalkyl, and Y is alkoxyalkyl.

In another embodiment of the compounds of formula ID, L³ is —S(O)₂—, L⁴ is —C(R⁴)₂—, R⁶ is alkoxyalkyl, and Y is alkoxyalkyl.

In another embodiment of the compounds of formula ID, L³ is —C(R⁴)₂—, L⁴ is —CH₂—, R⁶ is alkoxyalkyl, and Y is alkoxyalkyl.

In another embodiment of the compounds of formula ID, L³ is —CH₂—, L⁴ is —CH₂—, R⁶ is alkoxyalkyl, and Y is alkoxyalkyl.

In another embodiment of the compounds of formula ID, L³ is —CH(CH₃)—, L⁴ is —CH₂—, R⁶ is alkoxyalkyl, and Y is alkoxyalkyl.

In another embodiment of the compounds of formula ID, L³ is —C(O)—, L⁴ is —CH₂—, R⁶ is alkoxyalkyl, and Y is alkoxyalkyl.

In another embodiment of the compounds of formula ID, L³ is —S(O)₂—, L⁴ is —CH₂—, R⁶ is alkoxyalkyl, and Y is alkoxyalkyl.

In another embodiment of the compounds of formula ID, L³ is —C(R⁴)₂—, L⁴ is —CH(CH₃)—, R⁶ is alkoxyalkyl, and Y is alkoxyalkyl.

In another embodiment of the compounds of formula ID, L³ is —CH₂—, L⁴ is —CH(CH₃)—, R⁶ is alkoxyalkyl, and Y is alkoxyalkyl.

In another embodiment of the compounds of formula ID, L³ is —CH(CH₃)—, L⁴ is —CH(CH₃)—, R⁶ is alkoxyalkyl, and Y is alkoxyalkyl.

In another embodiment of the compounds of formula ID, L³ is —C(O)—, L⁴ is —CH(CH₃)—, R⁶ is alkoxyalkyl, and Y is alkoxyalkyl.

In another embodiment of the compounds of formula ID, L³ is —S(O)₂—, L⁴ is —CH(CH₃)—, R⁶ is alkoxyalkyl, and Y is alkoxyalkyl.

In another embodiment, the compounds of the present invention, or pharmaceutically acceptable salts, solvates, prodrugs, tautomers, or esters thereof, have the structure of formula (IE-1):

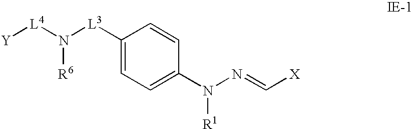

wherein Y-L⁴-N(R⁶)—, L³, R¹, and X are as defined herein.

In another embodiment of the compounds of formula IE-1, L⁴ is a covalent bond or —CH₂—; X is selected from the group consisting of substituted or unsubstituted phenyl, substituted or unsubstituted benzimidazolyl, and substituted or unsubstituted pyrimidyl; Y is selected from the group consisting of substituted or unsubstituted pyrimidyl, substituted or unsubstituted pyridyl, substituted or unsubstituted thienyl, substituted or unsubstituted phenyl, and dimethylaminomethyl; and R⁶ is selected from the group consisting of H, substituted or unsubstituted arylalkyl, and substituted or unsubstituted heteroarylalkyl.

In another embodiment of the compounds of formula IE-1, X is substituted or unsubstituted aryl.

In another embodiment of the compounds of formula IE-1, X is substituted or unsubstituted phenyl.

In another embodiment of the compounds of formula IE-1, X is substituted or unsubstituted naphthyl.

In another embodiment of the compounds of formula IE-1, X is substituted or unsubstituted heteroaryl.

In another embodiment of the compounds of formula IE-1, X is substituted or unsubstituted pyridyl.

In another embodiment of the compounds of formula IE-1, X is substituted or unsubstituted pyrimidyl.

In another embodiment of the compounds of formula IE-1, X is substituted or unsubstituted quinolyl.

In another embodiment of the compounds of formula IE-1, X is substituted or unsubstituted thienyl.

In another embodiment of the compounds of formula IE-1, X is substituted or unsubstituted furanyl.

In another embodiment of the compounds of formula IE-1, X is substituted or unsubstituted pyrrolyl.

In another embodiment of the compounds of formula IE-1, X is substituted or unsubstituted indolyl.

In another embodiment of the compounds of formula IE-1, X is substituted or unsubstituted benzimidazolyl.

In another embodiment of the compounds of formula IE-1, X is substituted or unsubstituted purinyl.

In another embodiment of the compounds of formula IE-1, R¹ is H.

In another embodiment of the compounds of formula IE-1, R¹ is alkyl.

In another embodiment of the compounds of formula IE-1, R¹ is methyl.

In another embodiment of the compounds of formula IE-1, R¹ is ethyl.

In another embodiment of the compounds of formula IE-1, R¹ is propyl.

In another embodiment of the compounds of formula IE-1, R¹ is substituted or unsubstituted benzyl.

In another embodiment of the compounds of formula IE-1, X is substituted or unsubstituted aryl, and R¹ is H.

In another embodiment of the compounds of formula IE-1, X is substituted or unsubstituted phenyl, and R¹ is H.

In another embodiment of the compounds of formula IE-1, X is substituted or unsubstituted naphthyl, and R¹ is H.

In another embodiment of the compounds of formula IE-1, X is substituted or unsubstituted heteroaryl, and R¹ is H.

In another embodiment of the compounds of formula IE-1, X is substituted or unsubstituted pyridyl, and R¹ is H.

In another embodiment of the compounds of formula IE-1, X is substituted or unsubstituted pyrimidyl, and R¹ is H.

In another embodiment of the compounds of formula IE-1, X is substituted or unsubstituted quinolyl, and R¹ is H.

In another embodiment of the compounds of formula IE-1, X is substituted or unsubstituted thienyl, and R¹ is H.

In another embodiment of the compounds of formula IE-1, X is substituted or unsubstituted furanyl, and R¹ is H.

In another embodiment of the compounds of formula IE-1, X is substituted or unsubstituted pyrrolyl, and R¹ is H.

In another embodiment of the compounds of formula IE-1, X is substituted or unsubstituted indolyl, and R¹ is H.

In another embodiment of the compounds of formula IE-1, X is substituted or unsubstituted benzimidazolyl, and R¹ is H.

In another embodiment of the compounds of formula IE-1, X is substituted or unsubstituted purinyl, and R¹ is H.

In another embodiment of the compounds of formula IE-1, X is substituted or unsubstituted aryl, and R¹ is alkyl.

In another embodiment of the compounds of formula IE-1, X is substituted or unsubstituted phenyl, and R¹ is alkyl.

In another embodiment of the compounds of formula IE-1, X is substituted or unsubstituted naphthyl, and R¹ is alkyl.

In another embodiment of the compounds of formula IE-1, X is substituted or unsubstituted heteroaryl, and R¹ is alkyl.

In another embodiment of the compounds of formula IE-1, X is substituted or unsubstituted pyridyl, and R¹ is alkyl.

In another embodiment of the compounds of formula IE-1, X is substituted or unsubstituted pyrimidyl, and R¹ is alkyl.

In another embodiment of the compounds of formula IE-1, X is substituted or unsubstituted quinolyl, and R¹ is alkyl.

In another embodiment of the compounds of formula IE-1, X is substituted or unsubstituted thienyl, and R¹ is alkyl.

In another embodiment of the compounds of formula IE-1, X is substituted or unsubstituted furanyl, and R¹ is alkyl.

In another embodiment of the compounds of formula IE-1, X is substituted or unsubstituted pyrrolyl, and R¹ is alkyl.

In another embodiment of the compounds of formula IE-1, X is substituted or unsubstituted indolyl, and R¹ is alkyl.

In another embodiment of the compounds of formula IE-1, X is substituted or unsubstituted benzimidazolyl, and R¹ is alkyl.

In another embodiment of the compounds of formula IE-1, X is substituted or unsubstituted purinyl, and R¹ is alkyl.

In another embodiment of the compounds of formula IE-1, X is substituted or unsubstituted aryl, and R¹ is methyl.

In another embodiment of the compounds of formula IE-1, X is substituted or unsubstituted phenyl, and R¹ is methyl.

In another embodiment of the compounds of formula IE-1, X is substituted or unsubstituted naphthyl, and R¹ is methyl.

In another embodiment of the compounds of formula IE-1, X is substituted or unsubstituted heteroaryl, and R¹ is methyl.

In another embodiment of the compounds of formula IE-1, X is substituted or unsubstituted pyridyl, and R¹ is methyl.

In another embodiment of the compounds of formula IE-1, X is substituted or unsubstituted pyrimidyl, and R¹ is methyl.

In another embodiment of the compounds of formula IE-1, X is substituted or unsubstituted quinolyl, and R¹ is methyl.

In another embodiment of the compounds of formula IE-1, X is substituted or unsubstituted thienyl, and R¹ is methyl.

In another embodiment of the compounds of formula IE-1, X is substituted or unsubstituted furanyl, and R¹ is methyl.

In another embodiment of the compounds of formula IE-1, X is substituted or unsubstituted pyrrolyl, and R¹ is methyl.

In another embodiment of the compounds of formula IE-1, X is substituted or unsubstituted indolyl, and R¹ is methyl.

In another embodiment of the compounds of formula IE-1, X is substituted or unsubstituted benzimidazolyl, and R¹ is methyl.

In another embodiment of the compounds of formula IE-1, X is substituted or unsubstituted purinyl, and R¹ is methyl.

In another embodiment of the compounds of formula IE-1, X is substituted or unsubstituted aryl, and R¹ is ethyl.

In another embodiment of the compounds of formula IE-1, X is substituted or unsubstituted phenyl, and R¹ is ethyl.

In another embodiment of the compounds of formula IE-1, X is substituted or unsubstituted naphthyl, and R¹ is ethyl.

In another embodiment of the compounds of formula IE-1, X is substituted or unsubstituted heteroaryl, and R¹ is ethyl.

In another embodiment of the compounds of formula IE-1, X is substituted or unsubstituted pyridyl, and R¹ is ethyl.

In another embodiment of the compounds of formula IE-1, X is substituted or unsubstituted pyrimidyl, and R¹ is ethyl.

In another embodiment of the compounds of formula IE-1, X is substituted or unsubstituted quinolyl, and R¹ is ethyl.

In another embodiment of the compounds of formula IE-1, X is substituted or unsubstituted thienyl, and R¹ is ethyl.

In another embodiment of the compounds of formula IE-1, X is substituted or unsubstituted furanyl, and R¹ is ethyl.

In another embodiment of the compounds of formula IE-1, X is substituted or unsubstituted pyrrolyl, and R¹ is ethyl.

In another embodiment of the compounds of formula IE-1, X is substituted or unsubstituted indolyl, and R¹ is ethyl.

In another embodiment of the compounds of formula IE-1, X is substituted or unsubstituted benzimidazolyl, and R¹ is ethyl.

In another embodiment of the compounds of formula IE-1, X is substituted or unsubstituted purinyl, and R¹ is ethyl.

In another embodiment of the compounds of formula IE-1, X is substituted or unsubstituted aryl, and R¹ is propyl.

In another embodiment of the compounds of formula IE-1, X is substituted or unsubstituted phenyl, and R¹ is propyl.

In another embodiment of the compounds of formula IE-1, X is substituted or unsubstituted naphthyl, and R¹ is propyl.

In another embodiment of the compounds of formula IE-1, X is substituted or unsubstituted heteroaryl, and R¹ is propyl.

In another embodiment of the compounds of formula IE-1, X is substituted or unsubstituted pyridyl, and R¹ is propyl.

In another embodiment of the compounds of formula IE-1, X is substituted or unsubstituted pyrimidyl, and R¹ is propyl.

In another embodiment of the compounds of formula IE-1, X is substituted or unsubstituted quinolyl, and R¹ is propyl.

In another embodiment of the compounds of formula IE-1, X is substituted or unsubstituted thienyl, and R¹ is propyl.

In another embodiment of the compounds of formula IE-1, X is substituted or unsubstituted furanyl, and R¹ is propyl.

In another embodiment of the compounds of formula IE-1, X is substituted or unsubstituted pyrrolyl, and R¹ is propyl.

In another embodiment of the compounds of formula IE-1, X is substituted or unsubstituted indolyl, and R¹ is propyl.

In another embodiment of the compounds of formula IE-1, X is substituted or unsubstituted benzimidazolyl, and R¹ is propyl.

In another embodiment of the compounds of formula IE-1, X is substituted or unsubstituted purinyl, and R¹ is propyl.

In another embodiment of the compounds of formula IE-1, X is substituted or unsubstituted aryl, and R¹ is substituted or unsubstituted benzyl.

In another embodiment of the compounds of formula IE-1, X is substituted or unsubstituted phenyl, and R¹ is substituted or unsubstituted benzyl.

In another embodiment of the compounds of formula IE-1, X is substituted or unsubstituted naphthyl, and R¹ is substituted or unsubstituted benzyl.

In another embodiment of the compounds of formula IE-1, X is substituted or unsubstituted heteroaryl, and R¹ is substituted or unsubstituted benzyl.

In another embodiment of the compounds of formula IE-1, X is substituted or unsubstituted pyridyl, and R¹ is substituted or unsubstituted benzyl.

In another embodiment of the compounds of formula IE-1, X is substituted or unsubstituted pyrimidyl, and R¹ is substituted or unsubstituted benzyl.

In another embodiment of the compounds of formula IE-1, X is substituted or unsubstituted quinolyl, and R¹ is substituted or unsubstituted benzyl.

In another embodiment of the compounds of formula IE-1, X is substituted or unsubstituted thienyl, and R¹ is substituted or unsubstituted benzyl.

In another embodiment of the compounds of formula IE-1, X is substituted or unsubstituted furanyl, and R¹ is substituted or unsubstituted benzyl.

In another embodiment of the compounds of formula IE-1, X is substituted or unsubstituted pyrrolyl, and R¹ is substituted or unsubstituted benzyl.

In another embodiment of the compounds of formula IE-1, X is substituted or unsubstituted indolyl, and R¹ is substituted or unsubstituted benzyl.

In another embodiment of the compounds of formula IE-1, X is substituted or unsubstituted benzimidazolyl, and R¹ is substituted or unsubstituted benzyl.

In another embodiment of the compounds of formula IE-1, X is substituted or unsubstituted purinyl, and R¹ is substituted or unsubstituted benzyl.

In another embodiment of the compounds of formula IE-1, L³ is —C(R⁴)₂—.

In another embodiment of the compounds of formula IE-1, L³ is —CH₂—.

In another embodiment of the compounds of formula IE-1, L³ is —CH(CH₃)—.

In another embodiment of the compounds of formula IE-1, L³ is —C(O)—.

In another embodiment of the compounds of formula IE-1, L³ is —S(O)₂—.

In another embodiment of the compounds of formula IE-1, L⁴ is —C(R⁴)₂—.

In another embodiment of the compounds of formula IE-1, L⁴ is —CH₂—.

In another embodiment of the compounds of formula IE-1, L⁴ is —CH(CH₃)—.

In another embodiment of the compounds of formula IE-1, L³ is —C(R⁴)₂—, and L⁴ is —C(R⁴)₂—.

In another embodiment of the compounds of formula IE-1, L³ is —CH₂—, and L⁴ is —C(R⁴)₂—.

In another embodiment of the compounds of formula IE-1, L³ is —CH(CH₃)—, and L⁴ is —C(R⁴)₂—.

In another embodiment of the compounds of formula IE-1, L³ is —C(O)—, and L⁴ is —C(R⁴)₂—.

In another embodiment of the compounds of formula IE-1, L³ is —S(O)₂—, and L⁴ is —C(R⁴)₂—.

In another embodiment of the compounds of formula IE-1, L³ is —C(R⁴)₂—, and L⁴ is —CH₂—.

In another embodiment of the compounds of formula IE-1, L³ is —CH₂—, and L⁴ is —CH₂—

In another embodiment of the compounds of formula IE-1, L³ is —CH(CH₃)—, and L⁴ is —CH₂—.

In another embodiment of the compounds of formula IE-1, L³ is —C(O)—, and L⁴ is —CH₂—.

In another embodiment of the compounds of formula IE-1, L³ is —S(O)₂—, and L⁴ is —CH₂—.

In another embodiment of the compounds of formula IE-1, L³ is —C(R⁴)₂—, and L⁴ is —CH(CH₃)—.

In another embodiment of the compounds of formula IE-1, L³ is —CH₂—, and L⁴ is —CH(CH₃)—.

In another embodiment of the compounds of formula IE-1, L³ is —CH(CH₃)—, and L⁴ is —CH(CH₃)—.

In another embodiment of the compounds of formula IE-1, L³ is —C(O)—, and L⁴ is —CH(CH₃)—.

In another embodiment of the compounds of formula IE-1, L³ is —S(O)₂—, and L⁴ is —CH(CH₃)—.

In another embodiment of the compounds of formula IE-1, L³ is —C(R⁴)₂—, L⁴ is —C(R⁴)₂—, and R⁶ is H.

In another embodiment of the compounds of formula IE-1, L³ is —CH₂—, L⁴ is —C(R⁴)₂—, and R⁶ is H.

In another embodiment of the compounds of formula IE-1, L³ is —CH(CH₃)—, L⁴ is —C(R⁴)₂—, and R¹ is H.

In another embodiment of the compounds of formula IE-1, L³ is —C(O)—, L⁴ is —C(R⁴)₂—, and R⁶ is H.

In another embodiment of the compounds of formula IE-1, L³ is —S(O)₂—, L⁴ is —C(R⁴)₂—, and R⁶ is H.

In another embodiment of the compounds of formula IE-1, L³ is —C(R⁴)₂—, L⁴ is —CH₂—, and R⁶ is H.

In another embodiment of the compounds of formula IE-1, L³ is —CH₂—, L⁴ is —CH₂—, and R⁶ is H.

In another embodiment of the compounds of formula IE-1, L³ is —CH(CH₃)—, L⁴ is —CH₂—, and R⁶ is H.

In another embodiment of the compounds of formula IE-1, L³ is —C(O)—, L⁴ is —CH₂—, and R⁶ is H.

In another embodiment of the compounds of formula IE-1, L³ is —S(O)₂—, L⁴ is —CH₂—, and R⁶ is H.

In another embodiment of the compounds of formula IE-1, L³ is —C(R⁴)₂—, L⁴ is —CH(CH₃)—, and R¹ is H.

In another embodiment of the compounds of formula IE-1, L³ is —CH₂—, L⁴ is —CH(CH₃)—, and R⁶ is H.

In another embodiment of the compounds of formula IE-1, L³ is —CH(CH₃)—, L⁴ is —CH(CH₃)—, and R⁶ is H.

In another embodiment of the compounds of formula IE-1, L³ is —C(O)—, L⁴ is —CH(CH₃)—, and R⁶ is H.

In another embodiment of the compounds of formula IE-1, L³ is —S(O)₂—, L⁴ is —CH(CH₃)—, and R⁶ is H.

In another embodiment of the compounds of formula IE-1, L³ is —C(R⁴)₂—, L⁴ is —C(R⁴)₂—, and R⁶ is alkyl.

In another embodiment of the compounds of formula IE-1, L³ is —CH₂—, L⁴ is —C(R⁴)₂—, and R⁶ is alkyl.

In another embodiment of the compounds of formula IE-1, L³ is —CH(CH₃)—, L⁴ is —C(R⁴)₂—, and R⁶ is alkyl.

In another embodiment of the compounds of formula IE-1, L³ is —C(O)—, L⁴ is —C(R⁴)₂—, and R⁶ is alkyl.

In another embodiment of the compounds of formula IE-1, L³ is —S(O)₂—, L⁴ is —C(R⁴)₂—, and R⁶ is alkyl.

In another embodiment of the compounds of formula IE-1, L³ is —C(R⁴)₂—, L⁴ is —CH₂—, and R⁶ is alkyl.

In another embodiment of the compounds of formula IE-1, L³ is —CH₂—, L⁴ is —CH₂—, and R⁶ is alkyl.

In another embodiment of the compounds of formula IE-1, L³ is —CH(CH₃)—, L⁴ is —CH₂—, and R⁶ is alkyl.

In another embodiment of the compounds of formula IE-1, L³ is —C(O)—, L⁴ is —CH₂—, and R⁶ is alkyl.

In another embodiment of the compounds of formula IE-1, L³ is —S(O)₂—, L⁴ is —CH₂—, and R⁶ is alkyl.

In another embodiment of the compounds of formula IE-1, L³ is —C(R⁴)₂—, L⁴ is —CH(CH₃)—, and R⁶ is alkyl.

In another embodiment of the compounds of formula IE-1, L³ is —CH₂—, L⁴ is —CH(CH₃)—, and R⁶ is alkyl.

In another embodiment of the compounds of formula IE-1, L³ is —CH(CH₃)—, L⁴ is —CH(CH₃)—, and R⁶ is alkyl.

In another embodiment of the compounds of formula IE-1, L³ is —C(O)—, L⁴ is —CH(CH₃)—, and R⁶ is alkyl.

In another embodiment of the compounds of formula IE-1, L³ is —S(O)₂—, L⁴ is —CH(CH₃)—, and R⁶ is alkyl.

In another embodiment of the compounds of formula IE-1, L³ is —C(R⁴)₂—, L⁴ is —C(R⁴)₂—, and R⁶ is methyl.

In another embodiment of the compounds of formula IE-1, L³ is —CH₂—, L⁴ is —C(R⁴)₂—, and R⁶ is methyl.

In another embodiment of the compounds of formula IE-1, L³ is —CH(CH₃)—, L⁴ is —C(R⁴)₂—, and R⁶ is methyl.

In another embodiment of the compounds of formula IE-1, L³ is —C(O)—, L⁴ is —C(R⁴)₂—, and R⁶ is methyl.

In another embodiment of the compounds of formula IE-1, L³ is —S(O)₂—, L⁴ is —C(R⁴)₂—, and R⁶ is methyl.

In another embodiment of the compounds of formula IE-1, L³ is —C(R⁴)₂—, L⁴ is —CH₂—, and R⁶ is methyl.

In another embodiment of the compounds of formula IE-1, L³ is —CH₂—, L⁴ is —CH₂—, and R⁶ is methyl.

In another embodiment of the compounds of formula IE-1, L³ is —CH(CH₃)—, L⁴ is —CH₂—, and R⁶ is methyl.

In another embodiment of the compounds of formula IE-1, L³ is —C(O)—, L⁴ is —CH₂—, and R⁶ is methyl.

In another embodiment of the compounds of formula IE-1, L³ is —S(O)₂—, L⁴ is —CH₂—, and R⁶ is methyl.

In another embodiment of the compounds of formula IE-1, L³ is —C(R⁴)₂—, L⁴ is —CH(CH₃)—, and R⁶ is methyl.

In another embodiment of the compounds of formula IE-1, L³ is —CH₂—, L⁴ is —CH(CH₃)—, and R⁶ is methyl.

In another embodiment of the compounds of formula IE-1, L³ is —CH(CH₃)—, L⁴ is —CH(CH₃)—, and R⁶ is methyl.

In another embodiment of the compounds of formula IE-1, L³ is —C(O)—, L⁴ is —CH(CH₃)—, and R⁶ is methyl.

In another embodiment of the compounds of formula IE-1, L³ is —S(O)₂—, L⁴ is —CH(CH₃)—, and R⁶ is methyl.

In another embodiment of the compounds of formula IE-1, L³ is —C(R⁴)₂—, L⁴ is —C(R⁴)₂—, and R⁶ is substituted or unsubstituted benzyl.

In another embodiment of the compounds of formula IE-1, L³ is —CH₂—, L⁴ is —C(R⁴)₂—, and R⁶ is substituted or unsubstituted benzyl.

In another embodiment of the compounds of formula IE-1, L³ is —CH(CH₃)—, L⁴ is —C(R⁴)₂—, and R⁶ is substituted or unsubstituted benzyl.

In another embodiment of the compounds of formula IE-1, L³ is —C(O)—, L⁴ is —C(R⁴)₂—, and R⁶ is substituted or unsubstituted benzyl.

In another embodiment of the compounds of formula IE-1, L³ is —S(O)₂—, L⁴ is —C(R⁴)₂—, and R⁶ is substituted or unsubstituted benzyl.

In another embodiment of the compounds of formula IE-1, L³ is —C(R⁴)₂—, L⁴ is —CH₂—, and R⁶ is substituted or unsubstituted benzyl.

In another embodiment of the compounds of formula IE-1, L³ is —CH₂—, L⁴ is —CH₂—, and R⁶ is substituted or unsubstituted benzyl.

In another embodiment of the compounds of formula IE-1, L³ is —CH(CH₃)—, L⁴ is —CH₂—, and R⁶ is substituted or unsubstituted benzyl.

In another embodiment of the compounds of formula IE-1, L³ is —C(O)—, L⁴ is —CH₂—, and R⁶ is substituted or unsubstituted benzyl.

In another embodiment of the compounds of formula IE-1, L³ is —S(O)₂—, L⁴ is —CH₂—, and R⁶ is substituted or unsubstituted benzyl.

In another embodiment of the compounds of formula IE-1, L³ is —C(R⁴)₂—, L⁴ is —CH(CH₃)—, and R⁶ is substituted or unsubstituted benzyl.

In another embodiment of the compounds of formula IE-1, L³ is —CH₂—, L⁴ is —CH(CH₃)—, and R⁶ is substituted or unsubstituted benzyl.

In another embodiment of the compounds of formula IE-1, L³ is —CH(CH₃)—, L⁴ is —CH(CH₃)—, and R⁶ is substituted or unsubstituted benzyl.

In another embodiment of the compounds of formula IE-1, L³ is —C(O)—, L⁴ is —CH(CH₃)—, and R⁶ is substituted or unsubstituted benzyl.

In another embodiment of the compounds of formula IE-1, L³ is —S(O)₂—, L⁴ is —CH(CH₃)—, and R⁶ is substituted or unsubstituted benzyl.

In another embodiment of the compounds of formula IE-1, L³ is —C(R⁴)₂—, L⁴ is —C(R⁴)₂—, R⁶ is H, and Y is alkoxyalkyl.

In another embodiment of the compounds of formula IE-1, L³ is —CH₂—, L⁴ is —C(R⁴)₂—, R⁶ is H, and Y is alkoxyalkyl.

In another embodiment of the compounds of formula IE-1, L³ is —C(R⁴)₂—, L⁴ is —CH₂—, R⁶ is H, and Y is alkoxyalkyl.

In another embodiment of the compounds of formula IE-1, L³ is —CH₂—, L⁴ is —CH₂—, R⁶ is H, and Y is alkoxyalkyl.

In another embodiment of the compounds of formula IE-1, L³ is —C(O)—, L⁴ is —C(R⁴)₂—, R⁶ is H, and Y is alkoxyalkyl.

In another embodiment of the compounds of formula IE-1, L³ is —C(O)—, L⁴ is —CH₂—, R⁶ is H, and Y is alkoxyalkyl.

In another embodiment of the compounds of formula IE-1, L³ is —S(O)₂—, L⁴ is —C(R⁴)₂—, R⁶ is H, and Y is alkoxyalkyl.

In another embodiment of the compounds of formula IE-1, L³ is —S(O)₂—, L⁴ is —CH₂—, R⁶ is H, and Y is alkoxyalkyl.

In another embodiment of the compounds of formula IE-1, L³ is —C(R⁴)₂—, L⁴ is —C(R⁴)₂—, R⁶ is alkyl, and Y is alkoxyalkyl.

In another embodiment of the compounds of formula IE-1, L³ is —CH₂—, L⁴ is —C(R⁴)₂—, R⁶ is alkyl, and Y is alkoxyalkyl.

In another embodiment of the compounds of formula IE-1, L³ is —C(R⁴)₂—, L⁴ is —CH₂—, R⁶ is alkyl, and Y is alkoxyalkyl.

In another embodiment of the compounds of formula IE-1, L³ is —CH₂—, L⁴ is —CH₂—, R⁶ is alkyl, and Y is alkoxyalkyl.

In another embodiment of the compounds of formula IE-1, L³ is —C(O)—, L⁴ is —C(R⁴)₂—, R⁶ is alkyl, and Y is alkoxyalkyl.

In another embodiment of the compounds of formula IE-1, L³ is —C(O)—, L⁴ is —CH₂—, R⁶ is alkyl, and Y is alkoxyalkyl.

In another embodiment of the compounds of formula IE-1, L³ is —S(O)₂—, L⁴ is —C(R⁴)₂—, R⁶ is alkyl, and Y is alkoxyalkyl.

In another embodiment of the compounds of formula IE-1, L³ is —S(O)₂—, L⁴ is —CH₂—, R⁶ is alkyl, and Y is alkoxyalkyl.

In another embodiment of the compounds of formula IE-1, L³ is —C(R⁴)₂—, L⁴ is —C(R⁴)₂—, R⁶ is methyl, and Y is alkoxyalkyl.

In another embodiment of the compounds of formula IE-1, L³ is —CH₂—, L⁴ is —C(R⁴)₂—, R⁶ is methyl, and Y is alkoxyalkyl.

In another embodiment of the compounds of formula IE-1, L³ is —C(R⁴)₂—, L⁴ is —CH₂—, R⁶ is methyl, and Y is alkoxyalkyl.

In another embodiment of the compounds of formula IE-1, L³ is —CH₂—, L⁴ is —CH₂—, R⁶ is methyl, and Y is alkoxyalkyl.

In another embodiment of the compounds of formula IE-1, L³ is —C(O)—, L⁴ is —C(R⁴)₂—, R⁶ is methyl, and Y is alkoxyalkyl.

In another embodiment of the compounds of formula IE-1, L³ is —C(O)—, L⁴ is —CH₂—, R⁶ is methyl, and Y is alkoxyalkyl.

In another embodiment of the compounds of formula IE-1, L³ is —S(O)₂—, L⁴ is —C(R⁴)₂—, R⁶ is methyl, and Y is alkoxyalkyl.

In another embodiment of the compounds of formula IE-1, L³ is —S(O)₂—, L⁴ is —CH₂—, R⁶ is methyl, and Y is alkoxyalkyl.

In another embodiment of the compounds of formula IE-1, L³ is —C(R⁴)₂—, L⁴ is —C(R⁴)₂—, R⁶ is substituted or unsubstituted arylalkyl, and Y is alkoxyalkyl.

In another embodiment of the compounds of formula IE-1, L³ is —CH₂—, L⁴ is —C(R⁴)₂—, R⁶ is substituted or unsubstituted arylalkyl, and Y is alkoxyalkyl.

In another embodiment of the compounds of formula IE-1, L³ is —C(R⁴)₂—, L⁴ is —CH₂—, R⁶ is substituted or unsubstituted arylalkyl, and Y is alkoxyalkyl.

In another embodiment of the compounds of formula IE-1, L³ is —CH₂—, L⁴ is —CH₂—, R⁶ is substituted or unsubstituted arylalkyl, and Y is alkoxyalkyl.

In another embodiment of the compounds of formula IE-1, L³ is —C(O)—, L⁴ is —C(R⁴)₂—, R⁶ is substituted or unsubstituted arylalkyl, and Y is alkoxyalkyl.

In another embodiment of the compounds of formula IE-1, L³ is —C(O)—, L⁴ is —CH₂—, R⁶ is substituted or unsubstituted arylalkyl, and Y is alkoxyalkyl.

In another embodiment of the compounds of formula IE-1, L³ is —S(O)₂—, L⁴ is —C(R⁴)₂—, R⁶ is substituted or unsubstituted arylalkyl, and Y is alkoxyalkyl.

In another embodiment of the compounds of formula IE-1, L³ is —S(O)₂—, L⁴ is —CH₂—, R⁶ is substituted or unsubstituted arylalkyl, and Y is alkoxyalkyl.

In another embodiment of the compounds of formula IE-1, L³ is —C(R⁴)₂—, L⁴ is —C(R⁴)₂—, R⁶ is alkoxyalkyl, and Y is alkoxyalkyl.

In another embodiment of the compounds of formula IE-1, L³ is —CH₂—, L⁴ is —C(R⁴)₂—, R⁶ is alkoxyalkyl, and Y is alkoxyalkyl.

In another embodiment of the compounds of formula IE-1, L³ is —C(R⁴)₂—, L⁴ is —CH₂—, R⁶ is alkoxyalkyl, and Y is alkoxyalkyl.

In another embodiment of the compounds of formula IE-1, L³ is —CH₂—, L⁴ is —CH₂—, R⁶ is alkoxyalkyl, and Y is alkoxyalkyl.

In another embodiment of the compounds of formula IE-1, L³ is —C(O)—, L⁴ is —C(R⁴)₂—, R⁶ is alkoxyalkyl, and Y is alkoxyalkyl.

In another embodiment of the compounds of formula IE-1, L³ is —C(O)—, L⁴ is —CH₂—, R⁶ is alkoxyalkyl, and Y is alkoxyalkyl.

In another embodiment of the compounds of formula IE-1, L³ is —S(O)₂—, L⁴ is —C(R⁴)₂—, R⁶ is alkoxyalkyl, and Y is alkoxyalkyl.

In another embodiment of the compounds of formula IE-1, L³ is —S(O)₂—, L⁴ is —CH₂—, R⁶ is alkoxyalkyl, and Y is alkoxyalkyl.

In another embodiment of the compounds of formula IE-1, L³ is —C(R⁴)₂—, L⁴ is —C(R⁴)₂—, R⁶ is methoxyalkyl, and Y is alkoxyalkyl.

In another embodiment of the compounds of formula IE-1, L³ is —CH₂—, L⁴ is —C(R⁴)₂—, R⁶ is methoxyalkyl, and Y is alkoxyalkyl.

In another embodiment of the compounds of formula IE-1, L³ is —C(R⁴)₂—, L⁴ is —CH₂—, R⁶ is methoxyalkyl, and Y is alkoxyalkyl.

In another embodiment of the compounds of formula IE-1, L³ is —CH₂—, L⁴ is —CH₂—, R⁶ is methoxyalkyl, and Y is alkoxyalkyl.

In another embodiment of the compounds of formula IE-1, L³ is —C(O)—, L⁴ is —C(R⁴)₂—, R⁶ is methoxyalkyl, and Y is alkoxyalkyl.

In another embodiment of the compounds of formula IE-1, L³ is —C(O)—, L⁴ is —CH₂—, R⁶ is methoxyalkyl, and Y is alkoxyalkyl.

In another embodiment of the compounds of formula IE-1, L³ is —S(O)₂—, L⁴ is —C(R⁴)₂—, R⁶ is methoxyalkyl, and Y is alkoxyalkyl.

In another embodiment of the compounds of formula IE-1, L³ is —S(O)₂—, L⁴ is —CH₂—, R⁶ is methoxyalkyl, and Y is alkoxyalkyl.

In another embodiment of the compounds of formula IE-1, L³ is —C(R⁴)₂—, L⁴ is —C(R⁴)₂—, R⁶ is substituted or unsubstituted benzyl, and Y is alkoxyalkyl.

In another embodiment of the compounds of formula IE-1, L³ is —CH₂—, L⁴ is —C(R⁴)₂—, R⁶ is substituted or unsubstituted benzyl, and Y is alkoxyalkyl.

In another embodiment of the compounds of formula IE-1, L³ is —C(R⁴)₂—, L⁴ is —CH₂—, R⁶ is substituted or unsubstituted benzyl, and Y is alkoxyalkyl.

In another embodiment of the compounds of formula IE-1, L³ is —CH₂—, L⁴ is —CH₂—, R⁶ is substituted or unsubstituted benzyl, and Y is alkoxyalkyl.

In another embodiment of the compounds of formula IE-1, L³ is —C(O)—, L⁴ is —C(R⁴)₂—, R⁶ is substituted or unsubstituted benzyl, and Y is alkoxyalkyl.

In another embodiment of the compounds of formula IE-1, L³ is —C(O)—, L⁴ is —CH₂—, R⁶ is substituted or unsubstituted benzyl, and Y is alkoxyalkyl.

In another embodiment of the compounds of formula IE-1, L³ is —S(O)₂—, L⁴ is —C(R⁴)₂—, R⁶ is substituted or unsubstituted benzyl, and Y is alkoxyalkyl.

In another embodiment of the compounds of formula IE-1, L³ is —S(O)₂—, L⁴ is —CH₂—, R⁶ is substituted or unsubstituted benzyl, and Y is alkoxyalkyl.

In another embodiment of the compounds of formula IE-1, L³ is —C(R⁴)₂—, L⁴ is —C(R⁴)₂—, R⁶ is H, and Y is methoxyalkyl.

In another embodiment of the compounds of formula IE-1, L³ is —CH₂—, L⁴ is —C(R⁴)₂—, R⁶ is H, and Y is methoxyalkyl.

In another embodiment of the compounds of formula IE-1, L³ is —C(R⁴)₂—, L⁴ is —CH₂—, R⁶ is H, and Y is methoxyalkyl.

In another embodiment of the compounds of formula IE-1, L³ is —CH₂—, L⁴ is —CH₂—, R⁶ is H, and Y is methoxyalkyl.

In another embodiment of the compounds of formula IE-1, L³ is —C(O)—, L⁴ is —C(R⁴)₂—, R⁶ is H, and Y is methoxyalkyl.

In another embodiment of the compounds of formula IE-1, L³ is —C(O)—, L⁴ is —CH₂—, R⁶ is H, and Y is methoxyalkyl.

In another embodiment of the compounds of formula IE-1, L³ is —S(O)₂—, L⁴ is —C(R⁴)₂—, R⁶ is H, and Y is methoxyalkyl.

In another embodiment of the compounds of formula IE-1, L³ is —S(O)₂—, L⁴ is —CH₂—, R⁶ is H, and Y is methoxyalkyl.

In another embodiment of the compounds of formula IE-1, L³ is —C(R⁴)₂—, L⁴ is —C(R⁴)₂—, R⁶ is alkyl, and Y is methoxyalkyl.

In another embodiment of the compounds of formula IE-1, L³ is —CH₂—, L⁴ is —C(R⁴)₂—, R⁶ is alkyl, and Y is methoxyalkyl.

In another embodiment of the compounds of formula IE-1, L³ is —C(R⁴)₂—, L⁴ is —CH₂—, R⁶ is alkyl, and Y is methoxyalkyl.

In another embodiment of the compounds of formula IE-1, L³ is —CH₂—, L⁴ is —CH₂—, R⁶ is alkyl, and Y is methoxyalkyl.

In another embodiment of the compounds of formula IE-1, L³ is —C(O)—, L⁴ is —C(R⁴)₂—, R⁶ is alkyl, and Y is methoxyalkyl.

In another embodiment of the compounds of formula IE-1, L³ is —C(O)—, L⁴ is —CH₂—, R⁶ is alkyl, and Y is methoxyalkyl.

In another embodiment of the compounds of formula IE-1, L³ is —S(O)₂—, L⁴ is —C(R⁴)₂—, R⁶ is alkyl, and Y is methoxyalkyl.

In another embodiment of the compounds of formula IE-1, L³ is —S(O)₂—, L⁴ is —CH₂—, R⁶ is alkyl, and Y is methoxyalkyl.

In another embodiment of the compounds of formula IE-1, L³ is —C(R⁴)₂—, L⁴ is —C(R⁴)₂—, R⁶ is methyl, and Y is methoxyalkyl.

In another embodiment of the compounds of formula IE-1, L³ is —CH₂—, L⁴ is —C(R⁴)₂—, R⁶ is methyl, and Y is methoxyalkyl.

In another embodiment of the compounds of formula IE-1, L³ is —C(R⁴)₂—, L⁴ is —CH₂—, R⁶ is methyl, and Y is methoxyalkyl.

In another embodiment of the compounds of formula IE-1, L³ is —CH₂—, L⁴ is —CH₂—, R⁶ is methyl, and Y is methoxyalkyl.

In another embodiment of the compounds of formula IE-1, L³ is —C(O)—, L⁴ is —C(R⁴)₂—, R⁶ is methyl, and Y is methoxyalkyl.

In another embodiment of the compounds of formula IE-1, L³ is —C(O)—, L⁴ is —CH₂—, R⁶ is methyl, and Y is methoxyalkyl.

In another embodiment of the compounds of formula IE-1, L³ is —S(O)₂—, L⁴ is —C(R⁴)₂—, R⁶ is methyl, and Y is methoxyalkyl.

In another embodiment of the compounds of formula IE-1, L³ is —S(O)₂—, L⁴ is —CH₂—, R⁶ is methyl, and Y is methoxyalkyl.

In another embodiment of the compounds of formula IE-1, L³ is —C(R⁴)₂—, L⁴ is —C(R⁴)₂—, R⁶ is substituted or unsubstituted arylalkyl, and Y is methoxyalkyl.

In another embodiment of the compounds of formula IE-1, L³ is —CH₂—, L⁴ is —C(R⁴)₂—, R⁶ is substituted or unsubstituted arylalkyl, and Y is methoxyalkyl.

In another embodiment of the compounds of formula IE-1, L³ is —C(R⁴)₂—, L⁴ is —CH₂—, R⁶ is substituted or unsubstituted arylalkyl, and Y is methoxyalkyl.

In another embodiment of the compounds of formula IE-1, L³ is —CH₂—, L⁴ is —CH₂—, R⁶ is substituted or unsubstituted arylalkyl, and Y is methoxyalkyl.

In another embodiment of the compounds of formula IE-1, L³ is —C(O)—, L⁴ is —C(R⁴)₂—, R⁶ is substituted or unsubstituted arylalkyl, and Y is methoxyalkyl.

In another embodiment of the compounds of formula IE-1, L³ is —C(O)—, L⁴ is —CH₂—, R⁶ is substituted or unsubstituted arylalkyl, and Y is methoxyalkyl.

In another embodiment of the compounds of formula IE-1, L³ is —S(O)₂—, L⁴ is —C(R⁴)₂—, R⁶ is substituted or unsubstituted arylalkyl, and Y is methoxyalkyl.

In another embodiment of the compounds of formula IE-1, L³ is —S(O)₂—, L⁴ is —CH₂—, R⁶ is substituted or unsubstituted arylalkyl, and Y is methoxyalkyl.

In another embodiment of the compounds of formula IE-1, L³ is —C(R⁴)₂—, L⁴ is —C(R⁴)₂—, R⁶ is alkoxyalkyl, and Y is methoxyalkyl.

In another embodiment of the compounds of formula IE-1, L³ is —CH₂—, L⁴ is —C(R⁴)₂—, R⁶ is alkoxyalkyl, and Y is methoxyalkyl.

In another embodiment of the compounds of formula IE-1, L³ is —C(R⁴)₂—, L⁴ is —CH₂—, R⁶ is alkoxyalkyl, and Y is methoxyalkyl.

In another embodiment of the compounds of formula IE-1, L³ is —CH₂—, L⁴ is —CH₂—, R⁶ is alkoxyalkyl, and Y is methoxyalkyl.

In another embodiment of the compounds of formula IE-1, L³ is —C(O)—, L⁴ is —C(R⁴)₂—, R⁶ is alkoxyalkyl, and Y is methoxyalkyl.

In another embodiment of the compounds of formula IE-1, L³ is —C(O)—, L⁴ is —CH₂—, R⁶ is alkoxyalkyl, and Y is methoxyalkyl.

In another embodiment of the compounds of formula IE-1, L³ is —S(O)₂—, L⁴ is —C(R⁴)₂—, R⁶ is alkoxyalkyl, and Y is methoxyalkyl.

In another embodiment of the compounds of formula IE-1, L³ is —S(O)₂—, L⁴ is —CH₂—, R⁶ is alkoxyalkyl, and Y is methoxyalkyl.

In another embodiment of the compounds of formula IE-1, L³ is —C(R⁴)₂—, L⁴ is —C(R⁴)₂—, R⁶ is methoxyalkyl, and Y is methoxyalkyl.

In another embodiment of the compounds of formula IE-1, L³ is —CH₂—, L⁴ is —C(R⁴)₂—, R⁶ is methoxyalkyl, and Y is methoxyalkyl.

In another embodiment of the compounds of formula IE-1, L³ is —C(R⁴)₂—, L⁴ is —CH₂—, R⁶ is methoxyalkyl, and Y is methoxyalkyl.

In another embodiment of the compounds of formula IE-1, L³ is —CH₂—, L⁴ is —CH₂—, R⁶ is methoxyalkyl, and Y is methoxyalkyl.

In another embodiment of the compounds of formula IE-1, L³ is —C(O)—, L⁴ is —C(R⁴)₂—, R⁶ is methoxyalkyl, and Y is methoxyalkyl.

In another embodiment of the compounds of formula IE-1, L³ is —C(O)—, L⁴ is —CH₂—, R⁶ is methoxyalkyl, and Y is methoxyalkyl.

In another embodiment of the compounds of formula IE-1, L³ is —S(O)₂—, L⁴ is —C(R⁴)₂—, R⁶ is methoxyalkyl, and Y is methoxyalkyl.

In another embodiment of the compounds of formula IE-1, L³ is —S(O)₂—, L⁴ is —CH₂—, R⁶ is methoxyalkyl, and Y is methoxyalkyl.

In another embodiment of the compounds of formula IE-1, L³ is —C(R⁴)₂—, L⁴ is —C(R⁴)₂—, R⁶ is substituted or unsubstituted benzyl, and Y is methoxyalkyl.

In another embodiment of the compounds of formula IE-1, L³ is —CH₂—, L⁴ is —C(R⁴)₂—, R⁶ is substituted or unsubstituted benzyl, and Y is methoxyalkyl.

In another embodiment of the compounds of formula IE-1, L³ is —C(R⁴)₂—, L⁴ is —CH₂—, R⁶ is substituted or unsubstituted benzyl, and Y is methoxyalkyl.

In another embodiment of the compounds of formula IE-1, L³ is —CH₂—, L⁴ is —CH₂—, R⁶ is substituted or unsubstituted benzyl, and Y is methoxyalkyl.

In another embodiment of the compounds of formula IE-1, L³ is —C(O)—, L⁴ is —C(R⁴)₂—, R⁶ is substituted or unsubstituted benzyl, and Y is methoxyalkyl.

In another embodiment of the compounds of formula IE-1, L³ is —C(O)—, L⁴ is —CH₂—, R⁶ is substituted or unsubstituted benzyl, and Y is methoxyalkyl.

In another embodiment of the compounds of formula IE-1, L³ is —S(O)₂—, L⁴ is —C(R⁴)₂—, R⁶ is substituted or unsubstituted benzyl, and Y is methoxyalkyl.

In another embodiment of the compounds of formula IE-1, L³ is —S(O)₂—, L⁴ is —CH₂—, R⁶ is substituted or unsubstituted benzyl, and Y is methoxyalkyl.

In another embodiment, the compounds of the present invention, or pharmaceutically acceptable salts, solvates, prodrugs, tautomers, or esters thereof, have the structure of formula (IF):

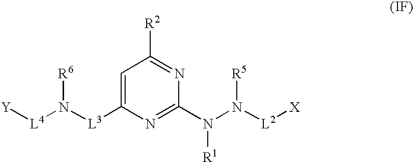

wherein R¹, R², R⁵, R⁶, L², L³, L⁴, X and Y are as defined herein.

In another embodiment of the compounds of formula IF, L² and L³ are each —C(R⁴)₂—; X and Y are each independently selected from the group consisting of substituted or unsubstituted aryl, and substituted or unsubstituted heteroaryl; R¹ and R⁵ are H; and R⁶ is selected from the group consisting of H, and substituted or unsubstituted cycloalkyl.

In another embodiment of the compounds of formula IF, L² is —(CR⁴)₂—; L³ is —C(O)—; L⁴ is a covalent bond or —(CR⁴)₂—; and R¹, R⁵, and R⁶ are each independently selected from the group consisting of H, substituted or unsubstituted arylalkyl, substituted or unsubstituted cycloalkyl, and substituted or unsubstituted heteroarylalkyl.

In another embodiment of the compounds of formula IF, L² is a covalent bond; L³ is —C(O)—; L⁴ is —C(R⁴)₂—; X is selected from the group consisting of substituted or unsubstituted aryl, and substituted or unsubstituted heteroaryl; Y is selected from the group consisting of substituted or unsubstituted aryl, and substituted or unsubstituted heteroaryl; R¹, R⁵, and R⁶ are each independently selected from the group consisting of H, substituted or unsubstituted arylalkyl, substituted or unsubstituted cycloalkyl, and substituted or unsubstituted heteroarylalkyl; and each R⁴ is H or alkyl.

In another embodiment of the compounds of formula IF, R⁵ and R¹, together with the nitrogen atoms to which they are each attached, form a substituted or unsubstituted heterocyclyl ring; X and Y are selected from the group consisting of substituted or unsubstituted aryl, and substituted or unsubstituted heteroaryl; each R⁴ is H or alkyl; and R⁶ is selected from the group consisting of H, substituted or unsubstituted arylalkyl, substituted or unsubstituted cycloalkyl, and substituted or unsubstituted heteroarylalkyl.

In another embodiment of the compounds of formula IF, L² is a covalent bond.

In another embodiment of the compounds of formula IF, L² is —C(R⁴)₂—.

In another embodiment of the compounds of formula IF, L² is —CH₂—.

In another embodiment of the compounds of formula IF, L² is —CH(CH₃)—.

In another embodiment of the compounds of formula IF, L² is —C(O)—.

In another embodiment of the compounds of formula IF, L² is —S(O)—.

In another embodiment of the compounds of formula IF, L² is —S(O)₂—.

In another embodiment of the compounds of formula IF, R² is H.

In another embodiment of the compounds of formula IF, R² is haloalkyl.

In another embodiment of the compounds of formula IF, R² is —CF₃.

In another embodiment of the compounds of formula IF, R¹ and R⁵ are independently H or alkyl.

In another embodiment of the compounds of formula IF, R¹ and R⁵ are independently H or methyl.

In another embodiment of the compounds of formula IF, R¹ and R⁵ are independently H or methyl.

In another embodiment of the compounds of formula IF, R¹ and R⁵ are independently H or substituted or unsubstituted arylalkyl.

In another embodiment of the compounds of formula IF, R¹ and R⁵ are both H.

In another embodiment of the compounds of formula IF, R¹ and R⁵, together with the nitrogen atoms to which they are shown attached, form a 5-membered heterocyclyl ring.

In another embodiment of the compounds of formula IF, R¹ and R⁵, together with the nitrogen atoms to which they are shown attached, form a 6-membered heterocyclyl ring.

In another embodiment of the compounds of formula IF, L² is a covalent bond, and R¹ and R⁵ are independently H or alkyl.

In another embodiment of the compounds of formula IF, L² is —C(R⁴)₂—, and R¹ and R⁵ are independently H or alkyl.

In another embodiment of the compounds of formula IF, L² is —CH₂—, and R¹ and R⁵ are independently H or alkyl.

In another embodiment of the compounds of formula IF, L² is —CH(CH₃)—, and R¹ and R⁵ are independently H or alkyl.

In another embodiment of the compounds of formula IF, L² is —C(O)—, and R¹ and R⁵ are independently H or alkyl.

In another embodiment of the compounds of formula IF, L² is a covalent bond, and R¹ and R⁵ are independently H or methyl.

In another embodiment of the compounds of formula IF, L² is —C(R⁴)₂—, and R¹ and R⁵ are independently H or methyl.

In another embodiment of the compounds of formula IF, L² is —CH₂—, and R¹ and R⁵ are independently H or methyl.

In another embodiment of the compounds of formula IF, L² is —CH(CH₃)—, and R¹ and R⁵ are independently H or methyl.

In another embodiment of the compounds of formula IF, L² is —C(O)—, and R¹ and R⁵ are independently H or methyl.

In another embodiment of the compounds of formula IF, L² is a covalent bond, and R¹ and R⁵ are independently H or substituted or unsubstituted arylalkyl.

In another embodiment of the compounds of formula IF, L² is —C(R⁴)₂—, and R¹ and R⁵ are independently H or substituted or unsubstituted arylalkyl.

In another embodiment of the compounds of formula IF, L² is —CH₂—, and R¹ and R⁵ are independently H or substituted or unsubstituted arylalkyl.

In another embodiment of the compounds of formula IF, L² is —CH(CH₃)—, and R¹ and R⁵ are independently H or substituted or unsubstituted arylalkyl.

In another embodiment of the compounds of formula IF, L² is —C(O)—, and R¹ and R⁵ are independently H or substituted or unsubstituted arylalkyl.

In another embodiment of the compounds of formula IF, L² is a covalent bond, and R¹ and R⁵ are both H.

In another embodiment of the compounds of formula IF, L² is —C(R⁴)₂—, and R¹ and R⁵ are both H.

In another embodiment of the compounds of formula IF, L² is —CH₂—, and R¹ and R⁵ are both H.

In another embodiment of the compounds of formula IF, L² is —CH(CH₃)—, and R¹ and R⁵ are both H.

In another embodiment of the compounds of formula IF, L² is —C(O)—, and R¹ and R⁵ are both H.

In another embodiment of the compounds of formula IF, L² is a covalent bond, and R¹ and R⁵, together with the nitrogen atoms to which they are shown attached, form a 5-membered heterocyclyl ring.

In another embodiment of the compounds of formula IF, L² is —C(R⁴)₂—, and R¹ and R⁵, together with the nitrogen atoms to which they are shown attached, form a 5-membered heterocyclyl ring.

In another embodiment of the compounds of formula IF, L² is —CH₂—, and R¹ and R⁵, together with the nitrogen atoms to which they are shown attached, form a 5-membered heterocyclyl ring.

In another embodiment of the compounds of formula IF, L² is —CH(CH₃)—, and R¹ and R⁵, together with the nitrogen atoms to which they are shown attached, form a 5-membered heterocyclyl ring.

In another embodiment of the compounds of formula IF, L² is —C(O)—, and R¹ and R⁵, together with the nitrogen atoms to which they are shown attached, form a 5-membered heterocyclyl ring.

In another embodiment of the compounds of formula IF, L² is a covalent bond, and R¹ and R⁵, together with the nitrogen atoms to which they are shown attached, form a 6-membered heterocyclyl ring.

In another embodiment of the compounds of formula IF, L² is —C(R⁴)₂—, and R¹ and R⁵, together with the nitrogen atoms to which they are shown attached, form a 6-membered heterocyclyl ring.

In another embodiment of the compounds of formula IF, L² is —CH₂—, and R¹ and R⁵, together with the nitrogen atoms to which they are shown attached, form a 6-membered heterocyclyl ring.

In another embodiment of the compounds of formula IF, L² is —CH(CH₃)—, and R¹ and R⁵, together with the nitrogen atoms to which they are shown attached, form a 6-membered heterocyclyl ring.

In another embodiment of the compounds of formula IF, L² is —C(O)—, and R¹ and R⁵, together with the nitrogen atoms to which they are shown attached, form a 6-membered heterocyclyl ring.

In another embodiment of the compounds of formula IF, L² is a covalent bond, R¹ and R⁵ are independently H or alkyl, and X is substituted or unsubstituted aryl.

In another embodiment of the compounds of formula IF, L² is —C(R⁴)₂—, R¹ and R⁵ are independently H or alkyl, and X is substituted or unsubstituted aryl.

In another embodiment of the compounds of formula IF, L² is —CH₂—, R¹ and R⁵ are independently H or alkyl, and X is substituted or unsubstituted aryl.

In another embodiment of the compounds of formula IF, L² is —CH(CH₃)—, R¹ and R⁵ are independently H or alkyl, and X is substituted or unsubstituted aryl.

In another embodiment of the compounds of formula IF, L² is —C(O)—, R¹ and R⁵ are independently H or alkyl, and X is substituted or unsubstituted aryl.

In another embodiment of the compounds of formula IF, L² is a covalent bond, R¹ and R⁵ are independently H or methyl, and X is substituted or unsubstituted aryl.

In another embodiment of the compounds of formula IF, L² is —C(R⁴)₂—, R¹ and R⁵ are independently H or methyl, and X is substituted or unsubstituted aryl.

In another embodiment of the compounds of formula IF, L² is —CH₂—, R¹ and R⁵ are independently H or methyl, and X is substituted or unsubstituted aryl.

In another embodiment of the compounds of formula IF, L² is —CH(CH₃)—, R¹ and R⁵ are independently H or methyl, and X is substituted or unsubstituted aryl.

In another embodiment of the compounds of formula IF, L² is —C(O)—, R¹ and R⁵ are independently H or methyl, and X is substituted or unsubstituted aryl.

In another embodiment of the compounds of formula IF, L² is a covalent bond, R¹ and R⁵ are independently H or substituted or unsubstituted arylalkyl, and X is substituted or unsubstituted aryl.

In another embodiment of the compounds of formula IF, L² is —C(R⁴)₂—, R¹ and R⁵ are independently H or substituted or unsubstituted arylalkyl, and X is substituted or unsubstituted aryl.

In another embodiment of the compounds of formula IF, L² is —CH₂—, R¹ and R⁵ are independently H or substituted or unsubstituted arylalkyl, and X is substituted or unsubstituted aryl.

In another embodiment of the compounds of formula IF, L² is —CH(CH₃)—, R¹ and R⁵ are independently H or substituted or unsubstituted arylalkyl, and X is substituted or unsubstituted aryl.

In another embodiment of the compounds of formula IF, L² is —C(O)—, R¹ and R⁵ are independently H or substituted or unsubstituted arylalkyl, and X is substituted or unsubstituted aryl.

In another embodiment of the compounds of formula IF, L² is a covalent bond, R¹ and R⁵ are both H, and X is substituted or unsubstituted aryl.

In another embodiment of the compounds of formula IF, L² is —C(R⁴)₂—, R¹ and R⁵ are both H, and X is substituted or unsubstituted aryl.

In another embodiment of the compounds of formula IF, L² is —CH₂—, R¹ and R⁵ are both H, and X is substituted or unsubstituted aryl.

In another embodiment of the compounds of formula IF, L² is —CH(CH₃)—, R¹ and R⁵ are both H, and X is substituted or unsubstituted aryl.

In another embodiment of the compounds of formula IF, L² is —C(O)—, R¹ and R⁵ are both H, and X is substituted or unsubstituted aryl.

In another embodiment of the compounds of formula IF, L² is a covalent bond, R¹ and R⁵, together with the nitrogen atoms to which they are shown attached, form a 5-membered heterocyclyl ring, and X is substituted or unsubstituted aryl.

In another embodiment of the compounds of formula IF, L² is —C(R⁴)₂—, R¹ and R⁵, together with the nitrogen atoms to which they are shown attached, form a 5-membered heterocyclyl ring, and X is substituted or unsubstituted aryl.

In another embodiment of the compounds of formula IF, L² is —CH₂—, R¹ and R⁵, together with the nitrogen atoms to which they are shown attached, form a 5-membered heterocyclyl ring, and X is substituted or unsubstituted aryl.

In another embodiment of the compounds of formula IF, L² is —CH(CH₃)—, R¹ and R⁵, together with the nitrogen atoms to which they are shown attached, form a 5-membered heterocyclyl ring, and X is substituted or unsubstituted aryl.

In another embodiment of the compounds of formula IF, L² is —C(O)—, R¹ and R⁵, together with the nitrogen atoms to which they are shown attached, form a 5-membered heterocyclyl ring, and X is substituted or unsubstituted aryl.

In another embodiment of the compounds of formula IF, L² is a covalent bond, R¹ and R⁵, together with the nitrogen atoms to which they are shown attached, form a 6-membered heterocyclyl ring, and X is substituted or unsubstituted aryl.

In another embodiment of the compounds of formula IF, L² is —C(R⁴)₂—, R¹ and R⁵, together with the nitrogen atoms to which they are shown attached, form a 6-membered heterocyclyl ring, and X is substituted or unsubstituted aryl.

In another embodiment of the compounds of formula IF, L² is —CH₂—, R¹ and R⁵, together with the nitrogen atoms to which they are shown attached, form a 6-membered heterocyclyl ring, and X is substituted or unsubstituted aryl.

In another embodiment of the compounds of formula IF, L² is —CH(CH₃)—, R¹ and R⁵, together with the nitrogen atoms to which they are shown attached, form a 6-membered heterocyclyl ring, and X is substituted or unsubstituted aryl.

In another embodiment of the compounds of formula IF, L² is —C(O)—, R¹ and R⁵, together with the nitrogen atoms to which they are shown attached, form a 6-membered heterocyclyl ring, and X is substituted or unsubstituted aryl.

In another embodiment of the compounds of formula IF, L² is a covalent bond, R¹ and R⁵ are independently H or alkyl, and X is substituted or unsubstituted phenyl.

In another embodiment of the compounds of formula IF, L² is —C(R⁴)₂—, R¹ and R⁵ are independently H or alkyl, and X is substituted or unsubstituted phenyl.

In another embodiment of the compounds of formula IF, L² is —CH₂—, R¹ and R⁵ are independently H or alkyl, and X is substituted or unsubstituted phenyl.

In another embodiment of the compounds of formula IF, L² is —CH(CH₃)—, R¹ and R⁵ are independently H or alkyl, and X is substituted or unsubstituted phenyl.

In another embodiment of the compounds of formula IF, L² is —C(O)—, R¹ and R⁵ are independently H or alkyl, and X is substituted or unsubstituted phenyl.

In another embodiment of the compounds of formula IF, L² is a covalent bond, R¹ and R⁵ are independently H or methyl, and X is substituted or unsubstituted phenyl.

In another embodiment of the compounds of formula IF, L² is —C(R⁴)₂—, R¹ and R⁵ are independently H or methyl, and X is substituted or unsubstituted phenyl.

In another embodiment of the compounds of formula IF, L² is —CH₂—, R¹ and R⁵ are independently H or methyl, and X is substituted or unsubstituted phenyl.

In another embodiment of the compounds of formula IF, L² is —CH(CH₃)—, R¹ and R⁵ are independently H or methyl, and X is substituted or unsubstituted phenyl.

In another embodiment of the compounds of formula IF, L² is —C(O)—, R¹ and R⁵ are independently H or methyl, and X is substituted or unsubstituted phenyl.

In another embodiment of the compounds of formula IF, L² is a covalent bond, R¹ and R⁵ are independently H or substituted or unsubstituted arylalkyl, and X is substituted or unsubstituted phenyl.

In another embodiment of the compounds of formula IF, L² is —C(R⁴)₂—, R¹ and R⁵ are independently H or substituted or unsubstituted arylalkyl, and X is substituted or unsubstituted phenyl.

In another embodiment of the compounds of formula IF, L² is —CH₂—, R¹ and R⁵ are independently H or substituted or unsubstituted arylalkyl, and X is substituted or unsubstituted phenyl.

In another embodiment of the compounds of formula IF, L² is —CH(CH₃)—, R¹ and R⁵ are independently H or substituted or unsubstituted arylalkyl, and X is substituted or unsubstituted phenyl.

In another embodiment of the compounds of formula IF, L² is —C(O)—, R¹ and R⁵ are independently H or substituted or unsubstituted arylalkyl, and X is substituted or unsubstituted phenyl.

In another embodiment of the compounds of formula IF, L² is a covalent bond, R¹ and R⁵ are both H, and X is substituted or unsubstituted phenyl.

In another embodiment of the compounds of formula IF, L² is —C(R⁴)₂—, R¹ and R⁵ are both H, and X is substituted or unsubstituted phenyl.

In another embodiment of the compounds of formula IF, L² is —CH₂—, R¹ and R⁵ are both H, and X is substituted or unsubstituted phenyl.

In another embodiment of the compounds of formula IF, L² is —CH(CH₃)—, R¹ and R⁵ are both H, and X is substituted or unsubstituted phenyl.

In another embodiment of the compounds of formula IF, L² is —C(O)—, R¹ and R⁵ are both H, and X is substituted or unsubstituted phenyl.

In another embodiment of the compounds of formula IF, L² is a covalent bond, R¹ and R⁵, together with the nitrogen atoms to which they are shown attached, form a 5-membered heterocyclyl ring, and X is substituted or unsubstituted phenyl.

In another embodiment of the compounds of formula IF, L² is —C(R⁴)₂—, R¹ and R⁵, together with the nitrogen atoms to which they are shown attached, form a 5-membered heterocyclyl ring, and X is substituted or unsubstituted phenyl.

In another embodiment of the compounds of formula IF, L² is —CH₂—, R¹ and R⁵, together with the nitrogen atoms to which they are shown attached, form a 5-membered heterocyclyl ring, and X is substituted or unsubstituted phenyl.

In another embodiment of the compounds of formula IF, L² is —CH(CH₃)—, R¹ and R⁵, together with the nitrogen atoms to which they are shown attached, form a 5-membered heterocyclyl ring, and X is substituted or unsubstituted phenyl.

In another embodiment of the compounds of formula IF, L² is —C(O)—, R¹ and R⁵, together with the nitrogen atoms to which they are shown attached, form a 5-membered heterocyclyl ring, and X is substituted or unsubstituted phenyl.

In another embodiment of the compounds of formula IF, L² is a covalent bond, R¹ and R⁵, together with the nitrogen atoms to which they are shown attached, form a 6-membered heterocyclyl ring, and X is substituted or unsubstituted phenyl.

In another embodiment of the compounds of formula IF, L² is —C(R⁴)₂—, R¹ and R⁵, together with the nitrogen atoms to which they are shown attached, form a 6-membered heterocyclyl ring, and X is substituted or unsubstituted phenyl.

In another embodiment of the compounds of formula IF, L² is —CH₂—, R¹ and R⁵, together with the nitrogen atoms to which they are shown attached, form a 6-membered heterocyclyl ring, and X is substituted or unsubstituted phenyl.

In another embodiment of the compounds of formula IF, L² is —CH(CH₃)—, R¹ and R⁵, together with the nitrogen atoms to which they are shown attached, form a 6-membered heterocyclyl ring, and X is substituted or unsubstituted phenyl.

In another embodiment of the compounds of formula IF, L² is —C(O)—, R¹ and R⁵, together with the nitrogen atoms to which they are shown attached, form a 6-membered heterocyclyl ring, and X is substituted or unsubstituted phenyl.

In another embodiment of the compounds of formula IF, L² is a covalent bond, R¹ and R⁵ are independently H or alkyl, and X is substituted or unsubstituted heteroaryl.

In another embodiment of the compounds of formula IF, L² is —C(R⁴)₂—, R¹ and R⁵ are independently H or alkyl, and X is substituted or unsubstituted heteroaryl.

In another embodiment of the compounds of formula IF, L² is —CH₂—, R¹ and R⁵ are independently H or alkyl, and X is substituted or unsubstituted heteroaryl.

In another embodiment of the compounds of formula IF, L² is —CH(CH₃)—, R¹ and R⁵ are independently H or alkyl, and X is substituted or unsubstituted heteroaryl.

In another embodiment of the compounds of formula IF, L² is —C(O)—, R¹ and R⁵ are independently H or alkyl, and X is substituted or unsubstituted heteroaryl.

In another embodiment of the compounds of formula IF, L² is a covalent bond, R¹ and R⁵ are independently H or methyl, and X is substituted or unsubstituted heteroaryl.

In another embodiment of the compounds of formula IF, L² is —C(R⁴)₂—, R¹ and R⁵ are independently H or methyl, and X is substituted or unsubstituted heteroaryl.

In another embodiment of the compounds of formula IF, L² is —CH₂—, R¹ and R⁵ are independently H or methyl, and X is substituted or unsubstituted heteroaryl.

In another embodiment of the compounds of formula IF, L² is —CH(CH₃)—, R¹ and R⁵ are independently H or methyl, and X is substituted or unsubstituted heteroaryl.

In another embodiment of the compounds of formula IF, L² is —C(O)—, R¹ and R⁵ are independently H or methyl, and X is substituted or unsubstituted heteroaryl.

In another embodiment of the compounds of formula IF, L² is a covalent bond, R¹ and R⁵ are independently H or substituted or unsubstituted arylalkyl, and X is substituted or unsubstituted heteroaryl.

In another embodiment of the compounds of formula IF, L² is —C(R⁴)₂—, R¹ and R⁵ are independently H or substituted or unsubstituted arylalkyl, and X is substituted or unsubstituted heteroaryl.

In another embodiment of the compounds of formula IF, L² is —CH₂—, R¹ and R⁵ are independently H or substituted or unsubstituted arylalkyl, and X is substituted or unsubstituted heteroaryl.

In another embodiment of the compounds of formula IF, L² is —CH(CH₃)—, R¹ and R⁵ are independently H or substituted or unsubstituted arylalkyl, and X is substituted or unsubstituted heteroaryl.

In another embodiment of the compounds of formula IF, L² is —C(O)—, R¹ and R⁵ are independently H or substituted or unsubstituted arylalkyl, and X is substituted or unsubstituted heteroaryl.

In another embodiment of the compounds of formula IF, L² is a covalent bond, R¹ and R⁵ are both H, and X is substituted or unsubstituted heteroaryl.

In another embodiment of the compounds of formula IF, L² is —C(R⁴)₂—, R¹ and R⁵ are both H, and X is substituted or unsubstituted heteroaryl.

In another embodiment of the compounds of formula IF, L² is —CH₂—, R¹ and R⁵ are both H, and X is substituted or unsubstituted heteroaryl.

In another embodiment of the compounds of formula IF, L² is —CH(CH₃)—, R¹ and R⁵ are both H, and X is substituted or unsubstituted heteroaryl.

In another embodiment of the compounds of formula IF, L² is —C(O)—, R¹ and R⁵ are both H, and X is substituted or unsubstituted heteroaryl.

In another embodiment of the compounds of formula IF, L² is a covalent bond, R¹ and R⁵, together with the nitrogen atoms to which they are shown attached, form a 5-membered heterocyclyl ring, and X is substituted or unsubstituted heteroaryl.

In another embodiment of the compounds of formula IF, L² is —C(R⁴)₂—, R¹ and R⁵, together with the nitrogen atoms to which they are shown attached, form a 5-membered heterocyclyl ring, and X is substituted or unsubstituted heteroaryl.

In another embodiment of the compounds of formula IF, L² is —CH₂—, R¹ and R⁵, together with the nitrogen atoms to which they are shown attached, form a 5-membered heterocyclyl ring, and X is substituted or unsubstituted heteroaryl.

In another embodiment of the compounds of formula IF, L² is —CH(CH₃)—, R¹ and R⁵, together with the nitrogen atoms to which they are shown attached, form a 5-membered heterocyclyl ring, and X is substituted or unsubstituted heteroaryl.

In another embodiment of the compounds of formula IF, L² is —C(O)—, R¹ and R⁵, together with the nitrogen atoms to which they are shown attached, form a 5-membered heterocyclyl ring, and X is substituted or unsubstituted heteroaryl.

In another embodiment of the compounds of formula IF, L² is a covalent bond, R¹ and R⁵, together with the nitrogen atoms to which they are shown attached, form a 6-membered heterocyclyl ring, and X is substituted or unsubstituted heteroaryl.

In another embodiment of the compounds of formula IF, L² is —C(R⁴)₂—, R¹ and R⁵, together with the nitrogen atoms to which they are shown attached, form a 6-membered heterocyclyl ring, and X is substituted or unsubstituted heteroaryl.

In another embodiment of the compounds of formula IF, L² is —CH₂—, R¹ and R⁵, together with the nitrogen atoms to which they are shown attached, form a 6-membered heterocyclyl ring, and X is substituted or unsubstituted heteroaryl.

In another embodiment of the compounds of formula IF, L² is —CH(CH₃)—, R¹ and R⁵, together with the nitrogen atoms to which they are shown attached, form a 6-membered heterocyclyl ring, and X is substituted or unsubstituted heteroaryl.

In another embodiment of the compounds of formula IF, L² is —C(O)—, R¹ and R⁵, together with the nitrogen atoms to which they are shown attached, form a 6-membered heterocyclyl ring, and X is substituted or unsubstituted heteroaryl.

In another embodiment of the compounds of formula IF, L² is a covalent bond, R¹ and R⁵ are independently H or alkyl, and X is substituted or unsubstituted pyridyl.

In another embodiment of the compounds of formula IF, L² is —C(R⁴)₂—, R¹ and R⁵ are independently H or alkyl, and X is substituted or unsubstituted pyridyl.

In another embodiment of the compounds of formula IF, L² is —CH₂—, R¹ and R⁵ are independently H or alkyl, and X is substituted or unsubstituted pyridyl.

In another embodiment of the compounds of formula IF, L² is —CH(CH₃)—, R¹ and R⁵ are independently H or alkyl, and X is substituted or unsubstituted pyridyl.

In another embodiment of the compounds of formula IF, L² is —C(O)—, R¹ and R⁵ are independently H or alkyl, and X is substituted or unsubstituted pyridyl.

In another embodiment of the compounds of formula IF, L² is a covalent bond, R¹ and R⁵ are independently H or methyl, and X is substituted or unsubstituted pyridyl.

In another embodiment of the compounds of formula IF, L² is —C(R⁴)₂—, R¹ and R⁵ are independently H or methyl, and X is substituted or unsubstituted pyridyl.

In another embodiment of the compounds of formula IF, L² is —CH₂—, R¹ and R⁵ are independently H or methyl, and X is substituted or unsubstituted pyridyl.

In another embodiment of the compounds of formula IF, L² is —CH(CH₃)—, R¹ and R⁵ are independently H or methyl, and X is substituted or unsubstituted pyridyl.

In another embodiment of the compounds of formula IF, L² is —C(O)—, R¹ and R⁵ are independently H or methyl, and X is substituted or unsubstituted pyridyl.

In another embodiment of the compounds of formula IF, L² is a covalent bond, R¹ and R⁵ are independently H or substituted or unsubstituted arylalkyl, and X is substituted or unsubstituted pyridyl.

In another embodiment of the compounds of formula IF, L² is —C(R⁴)₂—, R¹ and R⁵ are independently H or substituted or unsubstituted arylalkyl, and X is substituted or unsubstituted pyridyl.

In another embodiment of the compounds of formula IF, L² is —CH₂—, R¹ and R⁵ are independently H or substituted or unsubstituted arylalkyl, and X is substituted or unsubstituted pyridyl.

In another embodiment of the compounds of formula IF, L² is —CH(CH₃)—, R¹ and R⁵ are independently H or substituted or unsubstituted arylalkyl, and X is substituted or unsubstituted pyridyl.

In another embodiment of the compounds of formula IF, L² is —C(O)—, R¹ and R⁵ are independently H or substituted or unsubstituted arylalkyl, and X is substituted or unsubstituted pyridyl.

In another embodiment of the compounds of formula IF, L² is a covalent bond, R¹ and R⁵ are both H, and X is substituted or unsubstituted pyridyl.

In another embodiment of the compounds of formula IF, L² is —C(R⁴)₂—, R¹ and R⁵ are both H, and X is substituted or unsubstituted pyridyl.

In another embodiment of the compounds of formula IF, L² is —CH₂—, R¹ and R⁵ are both H, and X is substituted or unsubstituted pyridyl.

In another embodiment of the compounds of formula IF, L² is —CH(CH₃)—, R¹ and R⁵ are both H, and X is substituted or unsubstituted pyridyl.

In another embodiment of the compounds of formula IF, L² is —C(O)—, R¹ and R⁵ are both H, and X is substituted or unsubstituted pyridyl.

In another embodiment of the compounds of formula IF, L² is a covalent bond, R¹ and R⁵, together with the nitrogen atoms to which they are shown attached, form a 5-membered heterocyclyl ring, and X is substituted or unsubstituted pyridyl.

In another embodiment of the compounds of formula IF, L² is —C(R⁴)₂—, R¹ and R⁵, together with the nitrogen atoms to which they are shown attached, form a 5-membered heterocyclyl ring, and X is substituted or unsubstituted pyridyl.

In another embodiment of the compounds of formula IF, L² is —CH₂—, R¹ and R⁵, together with the nitrogen atoms to which they are shown attached, form a 5-membered heterocyclyl ring, and X is substituted or unsubstituted pyridyl.

In another embodiment of the compounds of formula IF, L² is —CH(CH₃)—, R¹ and R⁵, together with the nitrogen atoms to which they are shown attached, form a 5-membered heterocyclyl ring, and X is substituted or unsubstituted pyridyl.

In another embodiment of the compounds of formula IF, L² is —C(O)—, R¹ and R⁵, together with the nitrogen atoms to which they are shown attached, form a 5-membered heterocyclyl ring, and X is substituted or unsubstituted pyridyl.

In another embodiment of the compounds of formula IF, L² is a covalent bond, R¹ and R⁵, together with the nitrogen atoms to which they are shown attached, form a 6-membered heterocyclyl ring, and X is substituted or unsubstituted pyridyl.

In another embodiment of the compounds of formula IF, L² is —C(R⁴)₂—, R¹ and R⁵, together with the nitrogen atoms to which they are shown attached, form a 6-membered heterocyclyl ring, and X is substituted or unsubstituted pyridyl.

In another embodiment of the compounds of formula IF, L² is —CH₂—, R¹ and R⁵, together with the nitrogen atoms to which they are shown attached, form a 6-membered heterocyclyl ring, and X is substituted or unsubstituted pyridyl.

In another embodiment of the compounds of formula IF, L² is —CH(CH₃)—, R¹ and R⁵, together with the nitrogen atoms to which they are shown attached, form a 6-membered heterocyclyl ring, and X is substituted or unsubstituted pyridyl.

In another embodiment of the compounds of formula IF, L² is —C(O)—, R¹ and R⁵, together with the nitrogen atoms to which they are shown attached, form a 6-membered heterocyclyl ring, and X is substituted or unsubstituted pyridyl.

In another embodiment of the compounds of formula IF, L² is a covalent bond, R¹ and R⁵ are independently H or alkyl, and X is substituted or unsubstituted pyrimidyl.

In another embodiment of the compounds of formula IF, L² is —C(R⁴)₂—, R¹ and R⁵ are independently H or alkyl, and X is substituted or unsubstituted pyrimidyl.

In another embodiment of the compounds of formula IF, L² is —CH₂—, R¹ and R⁵ are independently H or alkyl, and X is substituted or unsubstituted pyrimidyl.

In another embodiment of the compounds of formula IF, L² is —CH(CH₃)—, R¹ and R⁵ are independently H or alkyl, and X is substituted or unsubstituted pyrimidyl.

In another embodiment of the compounds of formula IF, L² is —C(O)—, R¹ and R⁵ are independently H or alkyl, and X is substituted or unsubstituted pyrimidyl.

In another embodiment of the compounds of formula IF, L² is a covalent bond, R¹ and R⁵ are independently H or methyl, and X is substituted or unsubstituted pyrimidyl.

In another embodiment of the compounds of formula IF, L² is —C(R⁴)₂—, R¹ and R⁵ are independently H or methyl, and X is substituted or unsubstituted pyrimidyl.

In another embodiment of the compounds of formula IF, L² is —CH₂—, R¹ and R⁵ are independently H or methyl, and X is substituted or unsubstituted pyrimidyl.

In another embodiment of the compounds of formula IF, L² is —CH(CH₃)—, R¹ and R⁵ are independently H or methyl, and X is substituted or unsubstituted pyrimidyl.

In another embodiment of the compounds of formula IF, L² is —C(O)—, R¹ and R⁵ are independently H or methyl, and X is substituted or unsubstituted pyrimidyl.

In another embodiment of the compounds of formula IF, L² is a covalent bond, R¹ and R⁵ are independently H or substituted or unsubstituted arylalkyl, and X is substituted or unsubstituted pyrimidyl.

In another embodiment of the compounds of formula IF, L² is —C(R⁴)₂—, R¹ and R⁵ are independently H or substituted or unsubstituted arylalkyl, and X is substituted or unsubstituted pyrimidyl.

In another embodiment of the compounds of formula IF, L² is —CH₂—, R¹ and R⁵ are independently H or substituted or unsubstituted arylalkyl, and X is substituted or unsubstituted pyrimidyl.

In another embodiment of the compounds of formula IF, L² is —CH(CH₃)—, R¹ and R⁵ are independently H or substituted or unsubstituted arylalkyl, and X is substituted or unsubstituted pyrimidyl.

In another embodiment of the compounds of formula IF, L² is —C(O)—, R¹ and R⁵ are independently H or substituted or unsubstituted arylalkyl, and X is substituted or unsubstituted pyrimidyl.

In another embodiment of the compounds of formula IF, L² is a covalent bond, R¹ and R⁵ are both H, and X is substituted or unsubstituted pyrimidyl.

In another embodiment of the compounds of formula IF, L² is —C(R⁴)₂—, R¹ and R⁵ are both H, and X is substituted or unsubstituted pyrimidyl.

In another embodiment of the compounds of formula IF, L² is —CH₂—, R¹ and R⁵ are both H, and X is substituted or unsubstituted pyrimidyl.

In another embodiment of the compounds of formula IF, L² is —CH(CH₃)—, R¹ and R⁵ are both H, and X is substituted or unsubstituted pyrimidyl.

In another embodiment of the compounds of formula IF, L² is —C(O)—, R¹ and R⁵ are both H, and X is substituted or unsubstituted pyrimidyl.

In another embodiment of the compounds of formula IF, L² is a covalent bond, R¹ and R⁵, together with the nitrogen atoms to which they are shown attached, form a 5-membered heterocyclyl ring, and X is substituted or unsubstituted pyrimidyl.

In another embodiment of the compounds of formula IF, L² is —C(R⁴)₂—, R¹ and R⁵, together with the nitrogen atoms to which they are shown attached, form a 5-membered heterocyclyl ring, and X is substituted or unsubstituted pyrimidyl.

In another embodiment of the compounds of formula IF, L² is —CH₂—, R¹ and R⁵, together with the nitrogen atoms to which they are shown attached, form a 5-membered heterocyclyl ring, and X is substituted or unsubstituted pyrimidyl.

In another embodiment of the compounds of formula IF, L² is —CH(CH₃)—, R¹ and R⁵, together with the nitrogen atoms to which they are shown attached, form a 5-membered heterocyclyl ring, and X is substituted or unsubstituted pyrimidyl.

In another embodiment of the compounds of formula IF, L² is —C(O)—, R¹ and R⁵, together with the nitrogen atoms to which they are shown attached, form a 5-membered heterocyclyl ring, and X is substituted or unsubstituted pyrimidyl.

In another embodiment of the compounds of formula IF, L² is a covalent bond, R¹ and R⁵, together with the nitrogen atoms to which they are shown attached, form a 6-membered heterocyclyl ring, and X is substituted or unsubstituted pyrimidyl.

In another embodiment of the compounds of formula IF, L² is —C(R⁴)₂—, R¹ and R⁵, together with the nitrogen atoms to which they are shown attached, form a 6-membered heterocyclyl ring, and X is substituted or unsubstituted pyrimidyl.

In another embodiment of the compounds of formula IF, L² is —CH₂—, R¹ and R⁵, together with the nitrogen atoms to which they are shown attached, form a 6-membered heterocyclyl ring, and X is substituted or unsubstituted pyrimidyl.

In another embodiment of the compounds of formula IF, L² is —CH(CH₃)—, R¹ and R⁵, together with the nitrogen atoms to which they are shown attached, form a 6-membered heterocyclyl ring, and X is substituted or unsubstituted pyrimidyl.

In another embodiment of the compounds of formula IF, L² is —C(O)—, R¹ and R⁵, together with the nitrogen atoms to which they are shown attached, form a 6-membered heterocyclyl ring, and X is substituted or unsubstituted pyrimidyl.

In another embodiment of the compounds of formula IF, L² is a covalent bond, R¹ and R⁵ are independently H or alkyl, and X is substituted or unsubstituted indolyl.

In another embodiment of the compounds of formula IF, L² is —C(R⁴)₂—, R¹ and R⁵ are independently H or alkyl, and X is substituted or unsubstituted indolyl.

In another embodiment of the compounds of formula IF, L² is —CH₂—, R¹ and R⁵ are independently H or alkyl, and X is substituted or unsubstituted indolyl.

In another embodiment of the compounds of formula IF, L² is —CH(CH₃)—, R¹ and R⁵ are independently H or alkyl, and X is substituted or unsubstituted indolyl.

In another embodiment of the compounds of formula IF, L² is —C(O)—, R¹ and R⁵ are independently H or alkyl, and X is substituted or unsubstituted indolyl.

In another embodiment of the compounds of formula IF, L² is a covalent bond, R¹ and R⁵ are independently H or methyl, and X is substituted or unsubstituted indolyl.

In another embodiment of the compounds of formula IF, L² is —C(R⁴)₂—, R¹ and R⁵ are independently H or methyl, and X is substituted or unsubstituted indolyl.

In another embodiment of the compounds of formula IF, L² is —CH₂—, R¹ and R⁵ are independently H or methyl, and X is substituted or unsubstituted indolyl.

In another embodiment of the compounds of formula IF, L² is —CH(CH₃)—, R¹ and R⁵ are independently H or methyl, and X is substituted or unsubstituted indolyl.

In another embodiment of the compounds of formula IF, L² is —C(O)—, R¹ and R⁵ are independently H or methyl, and X is substituted or unsubstituted indolyl.

In another embodiment of the compounds of formula IF, L² is a covalent bond, R¹ and R⁵ are independently H or substituted or unsubstituted arylalkyl, and X is substituted or unsubstituted indolyl.

In another embodiment of the compounds of formula IF, L² is —C(R⁴)₂—, R¹ and R⁵ are independently H or substituted or unsubstituted arylalkyl, and X is substituted or unsubstituted indolyl.

In another embodiment of the compounds of formula IF, L² is —CH₂—, R¹ and R⁵ are independently H or substituted or unsubstituted arylalkyl, and X is substituted or unsubstituted indolyl.

In another embodiment of the compounds of formula IF, L² is —CH(CH₃)—, R¹ and R⁵ are independently H or substituted or unsubstituted arylalkyl, and X is substituted or unsubstituted indolyl.

In another embodiment of the compounds of formula IF, L² is —C(O)—, R¹ and R⁵ are independently H or substituted or unsubstituted arylalkyl, and X is substituted or unsubstituted indolyl.

In another embodiment of the compounds of formula IF, L² is a covalent bond, R¹ and R⁵ are both H, and X is substituted or unsubstituted indolyl.

In another embodiment of the compounds of formula IF, L² is —C(R⁴)₂—, R¹ and R⁵ are both H, and X is substituted or unsubstituted indolyl.

In another embodiment of the compounds of formula IF, L² is —CH₂—, R¹ and R⁵ are both H, and X is substituted or unsubstituted indolyl.

In another embodiment of the compounds of formula IF, L² is —CH(CH₃)—, R¹ and R⁵ are both H, and X is substituted or unsubstituted indolyl.

In another embodiment of the compounds of formula IF, L² is —C(O)—, R¹ and R⁵ are both H, and X is substituted or unsubstituted indolyl.

In another embodiment of the compounds of formula IF, L² is a covalent bond, R¹ and R⁵, together with the nitrogen atoms to which they are shown attached, form a 5-membered heterocyclyl ring, and X is substituted or unsubstituted indolyl.

In another embodiment of the compounds of formula IF, L² is —C(R⁴)₂—, R¹ and R⁵, together with the nitrogen atoms to which they are shown attached, form a 5-membered heterocyclyl ring, and X is substituted or unsubstituted indolyl.

In another embodiment of the compounds of formula IF, L² is —CH₂—, R¹ and R⁵, together with the nitrogen atoms to which they are shown attached, form a 5-membered heterocyclyl ring, and X is substituted or unsubstituted indolyl.

In another embodiment of the compounds of formula IF, L² is —CH(CH₃)—, R¹ and R⁵, together with the nitrogen atoms to which they are shown attached, form a 5-membered heterocyclyl ring, and X is substituted or unsubstituted indolyl.

In another embodiment of the compounds of formula IF, L² is —C(O)—, R¹ and R⁵, together with the nitrogen atoms to which they are shown attached, form a 5-membered heterocyclyl ring, and X is substituted or unsubstituted indolyl.

In another embodiment of the compounds of formula IF, L² is a covalent bond, R¹ and R⁵, together with the nitrogen atoms to which they are shown attached, form a 6-membered heterocyclyl ring, and X is substituted or unsubstituted indolyl.

In another embodiment of the compounds of formula IF, L² is —C(R⁴)₂—, R¹ and R⁵, together with the nitrogen atoms to which they are shown attached, form a 6-membered heterocyclyl ring, and X is substituted or unsubstituted indolyl.

In another embodiment of the compounds of formula IF, L² is —CH₂—, R¹ and R⁵, together with the nitrogen atoms to which they are shown attached, form a 6-membered heterocyclyl ring, and X is substituted or unsubstituted indolyl.

In another embodiment of the compounds of formula IF, L² is —CH(CH₃)—, R¹ and R⁵, together with the nitrogen atoms to which they are shown attached, form a 6-membered heterocyclyl ring, and X is substituted or unsubstituted indolyl.

In another embodiment of the compounds of formula IF, L² is —C(O)—, R¹ and R⁵, together with the nitrogen atoms to which they are shown attached, form a 6-membered heterocyclyl ring, and X is substituted or unsubstituted indolyl.

In another embodiment of the compounds of formula IF, L² is a covalent bond, R¹ and R⁵ are independently H or alkyl, and X is substituted or unsubstituted benzimidazolyl.

In another embodiment of the compounds of formula IF, L² is —C(R⁴)₂—, R¹ and R⁵ are independently H or alkyl, and X is substituted or unsubstituted benzimidazolyl.

In another embodiment of the compounds of formula IF, L² is —CH₂—, R¹ and R⁵ are independently H or alkyl, and X is substituted or unsubstituted benzimidazolyl.

In another embodiment of the compounds of formula IF, L² is —CH(CH₃)—, R¹ and R⁵ are independently H or alkyl, and X is substituted or unsubstituted benzimidazolyl.

In another embodiment of the compounds of formula IF, L² is —C(O)—, R¹ and R⁵ are independently H or alkyl, and X is substituted or unsubstituted benzimidazolyl.

In another embodiment of the compounds of formula IF, L² is a covalent bond, R¹ and R⁵ are independently H or methyl, and X is substituted or unsubstituted benzimidazolyl.

In another embodiment of the compounds of formula IF, L² is —C(R⁴)₂—, R¹ and R⁵ are independently H or methyl, and X is substituted or unsubstituted benzimidazolyl.

In another embodiment of the compounds of formula IF, L² is —CH₂—, R¹ and R⁵ are independently H or methyl, and X is substituted or unsubstituted benzimidazolyl.

In another embodiment of the compounds of formula IF, L² is —CH(CH₃)—, R¹ and R⁵ are independently H or methyl, and X is substituted or unsubstituted benzimidazolyl.

In another embodiment of the compounds of formula IF, L² is —C(O)—, R¹ and R⁵ are independently H or methyl, and X is substituted or unsubstituted benzimidazolyl.

In another embodiment of the compounds of formula IF, L² is a covalent bond, R¹ and R⁵ are independently H or substituted or unsubstituted arylalkyl, and X is substituted or unsubstituted benzimidazolyl.

In another embodiment of the compounds of formula IF, L² is —C(R⁴)₂—, R¹ and R⁵ are independently H or substituted or unsubstituted arylalkyl, and X is substituted or unsubstituted benzimidazolyl.

In another embodiment of the compounds of formula IF, L² is —CH₂—, R¹ and R⁵ are independently H or substituted or unsubstituted arylalkyl, and X is substituted or unsubstituted benzimidazolyl.

In another embodiment of the compounds of formula IF, L² is —CH(CH₃)—, R¹ and R⁵ are independently H or substituted or unsubstituted arylalkyl, and X is substituted or unsubstituted benzimidazolyl.

In another embodiment of the compounds of formula IF, L² is —C(O)—, R¹ and R⁵ are independently H or substituted or unsubstituted arylalkyl, and X is substituted or unsubstituted benzimidazolyl.

In another embodiment of the compounds of formula IF, L² is a covalent bond, R¹ and R⁵ are both H, and X is substituted or unsubstituted benzimidazolyl.

In another embodiment of the compounds of formula IF, L² is —C(R⁴)₂—, R¹ and R⁵ are both H, and X is substituted or unsubstituted benzimidazolyl.

In another embodiment of the compounds of formula IF, L² is —CH₂—, R¹ and R⁵ are both H, and X is substituted or unsubstituted benzimidazolyl.

In another embodiment of the compounds of formula IF, L² is —CH(CH₃)—, R¹ and R⁵ are both H, and X is substituted or unsubstituted benzimidazolyl.

In another embodiment of the compounds of formula IF, L² is —C(O)—, R¹ and R⁵ are both H, and X is substituted or unsubstituted benzimidazolyl.

In another embodiment of the compounds of formula IF, L² is a covalent bond, R¹ and R⁵, together with the nitrogen atoms to which they are shown attached, form a 5-membered heterocyclyl ring, and X is substituted or unsubstituted benzimidazolyl.

In another embodiment of the compounds of formula IF, L² is —C(R⁴)₂—, R¹ and R⁵, together with the nitrogen atoms to which they are shown attached, form a 5-membered heterocyclyl ring, and X is substituted or unsubstituted benzimidazolyl.

In another embodiment of the compounds of formula IF, L² is —CH₂—, R¹ and R⁵, together with the nitrogen atoms to which they are shown attached, form a 5-membered heterocyclyl ring, and X is substituted or unsubstituted benzimidazolyl.

In another embodiment of the compounds of formula IF, L² is —CH(CH₃)—, R¹ and R⁵, together with the nitrogen atoms to which they are shown attached, form a 5-membered heterocyclyl ring, and X is substituted or unsubstituted benzimidazolyl.

In another embodiment of the compounds of formula IF, L² is —C(O)—, R¹ and R⁵, together with the nitrogen atoms to which they are shown attached, form a 5-membered heterocyclyl ring, and X is substituted or unsubstituted benzimidazolyl.

In another embodiment of the compounds of formula IF, L² is a covalent bond, R¹ and R⁵, together with the nitrogen atoms to which they are shown attached, form a 6-membered heterocyclyl ring, and X is substituted or unsubstituted benzimidazolyl.

In another embodiment of the compounds of formula IF, L² is —C(R⁴)₂—, R¹ and R⁵, together with the nitrogen atoms to which they are shown attached, form a 6-membered heterocyclyl ring, and X is substituted or unsubstituted benzimidazolyl.

In another embodiment of the compounds of formula IF, L² is —CH₂—, R¹ and R⁵, together with the nitrogen atoms to which they are shown attached, form a 6-membered heterocyclyl ring, and X is substituted or unsubstituted benzimidazolyl.

In another embodiment of the compounds of formula IF, L² is —CH(CH₃)—, R¹ and R⁵, together with the nitrogen atoms to which they are shown attached, form a 6-membered heterocyclyl ring, and X is substituted or unsubstituted benzimidazolyl.

In another embodiment of the compounds of formula IF, L² is —C(O)—, R¹ and R⁵, together with the nitrogen atoms to which they are shown attached, form a 6-membered heterocyclyl ring, and X is substituted or unsubstituted benzimidazolyl.

In another embodiment of the compounds of formula IF, L² is a covalent bond, R¹ and R⁵ are independently H or alkyl, and X is substituted or unsubstituted purinyl.

In another embodiment of the compounds of formula IF, L² is —C(R⁴)₂—, R¹ and R⁵ are independently H or alkyl, and X is substituted or unsubstituted purinyl.

In another embodiment of the compounds of formula IF, L² is —CH₂—, R¹ and R⁵ are independently H or alkyl, and X is substituted or unsubstituted purinyl.

In another embodiment of the compounds of formula IF, L² is —CH(CH₃)—, R¹ and R⁵ are independently H or alkyl, and X is substituted or unsubstituted purinyl.

In another embodiment of the compounds of formula IF, L² is —C(O)—, R¹ and R⁵ are independently H or alkyl, and X is substituted or unsubstituted purinyl.

In another embodiment of the compounds of formula IF, L² is a covalent bond, R¹ and R⁵ are independently H or methyl, and X is substituted or unsubstituted purinyl.

In another embodiment of the compounds of formula IF, L² is —C(R⁴)₂—, R¹ and R⁵ are independently H or methyl, and X is substituted or unsubstituted purinyl.

In another embodiment of the compounds of formula IF, L² is —CH₂—, R¹ and R⁵ are independently H or methyl, and X is substituted or unsubstituted purinyl.

In another embodiment of the compounds of formula IF, L² is —CH(CH₃)—, R¹ and R⁵ are independently H or methyl, and X is substituted or unsubstituted purinyl.

In another embodiment of the compounds of formula IF, L² is —C(O)—, R¹ and R⁵ are independently H or methyl, and X is substituted or unsubstituted purinyl.

In another embodiment of the compounds of formula IF, L² is a covalent bond, R¹ and R⁵ are independently H or substituted or unsubstituted arylalkyl, and X is substituted or unsubstituted purinyl.

In another embodiment of the compounds of formula IF, L² is —C(R⁴)₂—, R¹ and R⁵ are independently H or substituted or unsubstituted arylalkyl, and X is substituted or unsubstituted purinyl.

In another embodiment of the compounds of formula IF, L² is —CH₂—, R¹ and R⁵ are independently H or substituted or unsubstituted arylalkyl, and X is substituted or unsubstituted purinyl.

In another embodiment of the compounds of formula IF, L² is —CH(CH₃)—, R¹ and R⁵ are independently H or substituted or unsubstituted arylalkyl, and X is substituted or unsubstituted purinyl.

In another embodiment of the compounds of formula IF, L² is —C(O)—, R¹ and R⁵ are independently H or substituted or unsubstituted arylalkyl, and X is substituted or unsubstituted purinyl.

In another embodiment of the compounds of formula IF, L² is a covalent bond, R¹ and R⁵ are both H, and X is substituted or unsubstituted purinyl.

In another embodiment of the compounds of formula IF, L² is —C(R⁴)₂—, R¹ and R⁵ are both H, and X is substituted or unsubstituted purinyl.

In another embodiment of the compounds of formula IF, L² is —CH₂—, R¹ and R⁵ are both H, and X is substituted or unsubstituted purinyl.

In another embodiment of the compounds of formula IF, L² is —CH(CH₃)—, R¹ and R⁵ are both H, and X is substituted or unsubstituted purinyl.

In another embodiment of the compounds of formula IF, L² is —C(O)—, R¹ and R⁵ are both H, and X is substituted or unsubstituted purinyl.

In another embodiment of the compounds of formula IF, L² is a covalent bond, R¹ and R⁵, together with the nitrogen atoms to which they are shown attached, form a 5-membered heterocyclyl ring, and X is substituted or unsubstituted purinyl.

In another embodiment of the compounds of formula IF, L² is —C(R⁴)₂—, R¹ and R⁵, together with the nitrogen atoms to which they are shown attached, form a 5-membered heterocyclyl ring, and X is substituted or unsubstituted purinyl.

In another embodiment of the compounds of formula IF, L² is —CH₂—, R¹ and R⁵, together with the nitrogen atoms to which they are shown attached, form a 5-membered heterocyclyl ring, and X is substituted or unsubstituted purinyl.

In another embodiment of the compounds of formula IF, L² is —CH(CH₃)—, R¹ and R⁵, together with the nitrogen atoms to which they are shown attached, form a 5-membered heterocyclyl ring, and X is substituted or unsubstituted purinyl.

In another embodiment of the compounds of formula IF, L² is —C(O)—, R¹ and R⁵, together with the nitrogen atoms to which they are shown attached, form a 5-membered heterocyclyl ring, and X is substituted or unsubstituted purinyl.

In another embodiment of the compounds of formula IF, L² is a covalent bond, R¹ and R⁵, together with the nitrogen atoms to which they are shown attached, form a 6-membered heterocyclyl ring, and X is substituted or unsubstituted purinyl.

In another embodiment of the compounds of formula IF, L² is —C(R⁴)₂—, R¹ and R⁵, together with the nitrogen atoms to which they are shown attached, form a 6-membered heterocyclyl ring, and X is substituted or unsubstituted purinyl.

In another embodiment of the compounds of formula IF, L² is —CH₂—, R¹ and R⁵, together with the nitrogen atoms to which they are shown attached, form a 6-membered heterocyclyl ring, and X is substituted or unsubstituted purinyl.

In another embodiment of the compounds of formula IF, L² is —CH(CH₃)—, R¹ and R⁵, together with the nitrogen atoms to which they are shown attached, form a 6-membered heterocyclyl ring, and X is substituted or unsubstituted purinyl.

In another embodiment of the compounds of formula IF, L² is —C(O)—, R¹ and R⁵, together with the nitrogen atoms to which they are shown attached, form a 6-membered heterocyclyl ring, and X is substituted or unsubstituted purinyl.

In another embodiment of the compounds of formula IF, L² is a covalent bond, R¹ and R⁵ are independently H or alkyl, and X is substituted or unsubstituted thienyl.

In another embodiment of the compounds of formula IF, L² is —C(R⁴)₂—, R¹ and R⁵ are independently H or alkyl, and X is substituted or unsubstituted thienyl.

In another embodiment of the compounds of formula IF, L² is —CH₂—, R¹ and R⁵ are independently H or alkyl, and X is substituted or unsubstituted thienyl.

In another embodiment of the compounds of formula IF, L² is —CH(CH₃)—, R¹ and R⁵ are independently H or alkyl, and X is substituted or unsubstituted thienyl.

In another embodiment of the compounds of formula IF, L² is —C(O)—, R¹ and R⁵ are independently H or alkyl, and X is substituted or unsubstituted thienyl.

In another embodiment of the compounds of formula IF, L² is a covalent bond, R¹ and R⁵ are independently H or methyl, and X is substituted or unsubstituted thienyl.

In another embodiment of the compounds of formula IF, L² is —C(R⁴)₂—, R¹ and R⁵ are independently H or methyl, and X is substituted or unsubstituted thienyl.

In another embodiment of the compounds of formula IF, L² is —CH₂—, R¹ and R⁵ are independently H or methyl, and X is substituted or unsubstituted thienyl.

In another embodiment of the compounds of formula IF, L² is —CH(CH₃)—, R¹ and R⁵ are independently H or methyl, and X is substituted or unsubstituted thienyl.

In another embodiment of the compounds of formula IF, L² is —C(O)—, R¹ and R⁵ are independently H or methyl, and X is substituted or unsubstituted thienyl.

In another embodiment of the compounds of formula IF, L² is a covalent bond, R¹ and R⁵ are independently H or substituted or unsubstituted arylalkyl, and X is substituted or unsubstituted thienyl.

In another embodiment of the compounds of formula IF, L² is —C(R⁴)₂—, R¹ and R⁵ are independently H or substituted or unsubstituted arylalkyl, and X is substituted or unsubstituted thienyl.

In another embodiment of the compounds of formula IF, L² is —CH₂—, R¹ and R⁵ are independently H or substituted or unsubstituted arylalkyl, and X is substituted or unsubstituted thienyl.

In another embodiment of the compounds of formula IF, L² is —CH(CH₃)—, R¹ and R⁵ are independently H or substituted or unsubstituted arylalkyl, and X is substituted or unsubstituted thienyl.

In another embodiment of the compounds of formula IF, L² is —C(O)—, R¹ and R⁵ are independently H or substituted or unsubstituted arylalkyl, and X is substituted or unsubstituted thienyl.

In another embodiment of the compounds of formula IF, L² is a covalent bond, R¹ and R⁵ are both H, and X is substituted or unsubstituted thienyl.

In another embodiment of the compounds of formula IF, L² is —C(R⁴)₂—, R¹ and R⁵ are both H, and X is substituted or unsubstituted thienyl.

In another embodiment of the compounds of formula IF, L² is —CH₂—, R¹ and R⁵ are both H, and X is substituted or unsubstituted thienyl.

In another embodiment of the compounds of formula IF, L² is —CH(CH₃)—, R¹ and R⁵ are both H, and X is substituted or unsubstituted thienyl.

In another embodiment of the compounds of formula IF, L² is —C(O)—, R¹ and R⁵ are both H, and X is substituted or unsubstituted thienyl.

In another embodiment of the compounds of formula IF, L² is a covalent bond, R¹ and R⁵, together with the nitrogen atoms to which they are shown attached, form a 5-membered heterocyclyl ring, and X is substituted or unsubstituted thienyl.

In another embodiment of the compounds of formula IF, L² is —C(R⁴)₂—, R¹ and R⁵, together with the nitrogen atoms to which they are shown attached, form a 5-membered heterocyclyl ring, and X is substituted or unsubstituted thienyl.

In another embodiment of the compounds of formula IF, L² is —CH₂—, R¹ and R⁵, together with the nitrogen atoms to which they are shown attached, form a 5-membered heterocyclyl ring, and X is substituted or unsubstituted thienyl.

In another embodiment of the compounds of formula IF, L² is —CH(CH₃)—, R¹ and R⁵, together with the nitrogen atoms to which they are shown attached, form a 5-membered heterocyclyl ring, and X is substituted or unsubstituted thienyl.

In another embodiment of the compounds of formula IF, L² is —C(O)—, R¹ and R⁵, together with the nitrogen atoms to which they are shown attached, form a 5-membered heterocyclyl ring, and X is substituted or unsubstituted thienyl.

In another embodiment of the compounds of formula IF, L² is a covalent bond, R¹ and R⁵, together with the nitrogen atoms to which they are shown attached, form a 6-membered heterocyclyl ring, and X is substituted or unsubstituted thienyl.

In another embodiment of the compounds of formula IF, L² is —C(R⁴)₂—, R¹ and R⁵, together with the nitrogen atoms to which they are shown attached, form a 6-membered heterocyclyl ring, and X is substituted or unsubstituted thienyl.

In another embodiment of the compounds of formula IF, L² is —CH₂—, R¹ and R⁵, together with the nitrogen atoms to which they are shown attached, form a 6-membered heterocyclyl ring, and X is substituted or unsubstituted thienyl.

In another embodiment of the compounds of formula IF, L² is —CH(CH₃)—, R¹ and R⁵, together with the nitrogen atoms to which they are shown attached, form a 6-membered heterocyclyl ring, and X is substituted or unsubstituted thienyl.

In another embodiment of the compounds of formula IF, L² is —C(O)—, R¹ and R⁵, together with the nitrogen atoms to which they are shown attached, form a 6-membered heterocyclyl ring, and X is substituted or unsubstituted thienyl.

In another embodiment of the compounds of formula IF, L² is a covalent bond, R¹ and R⁵ are independently H or alkyl, and X is substituted or unsubstituted furanyl.

In another embodiment of the compounds of formula IF, L² is —C(R⁴)₂—, R¹ and R⁵ are independently H or alkyl, and X is substituted or unsubstituted furanyl.

In another embodiment of the compounds of formula IF, L² is —CH₂—, R¹ and R⁵ are independently H or alkyl, and X is substituted or unsubstituted furanyl.

In another embodiment of the compounds of formula IF, L² is —CH(CH₃)—, R¹ and R⁵ are independently H or alkyl, and X is substituted or unsubstituted furanyl.

In another embodiment of the compounds of formula IF, L² is —C(O)—, R¹ and R⁵ are independently H or alkyl, and X is substituted or unsubstituted furanyl.

In another embodiment of the compounds of formula IF, L² is a covalent bond, R¹ and R⁵ are independently H or methyl, and X is substituted or unsubstituted furanyl.

In another embodiment of the compounds of formula IF, L² is —C(R⁴)₂—, R¹ and R⁵ are independently H or methyl, and X is substituted or unsubstituted furanyl.

In another embodiment of the compounds of formula IF, L² is —CH₂—, R¹ and R⁵ are independently H or methyl, and X is substituted or unsubstituted furanyl.

In another embodiment of the compounds of formula IF, L² is —CH(CH₃)—, R¹ and R⁵ are independently H or methyl, and X is substituted or unsubstituted furanyl.

In another embodiment of the compounds of formula IF, L² is —C(O)—, R¹ and R⁵ are independently H or methyl, and X is substituted or unsubstituted furanyl.

In another embodiment of the compounds of formula IF, L² is a covalent bond, R¹ and R⁵ are independently H or substituted or unsubstituted arylalkyl, and X is substituted or unsubstituted furanyl.

In another embodiment of the compounds of formula IF, L² is —C(R⁴)₂—, R¹ and R⁵ are independently H or substituted or unsubstituted arylalkyl, and X is substituted or unsubstituted furanyl.

In another embodiment of the compounds of formula IF, L² is —CH₂—, R¹ and R⁵ are independently H or substituted or unsubstituted arylalkyl, and X is substituted or unsubstituted furanyl.

In another embodiment of the compounds of formula IF, L² is —CH(CH₃)—, R¹ and R⁵ are independently H or substituted or unsubstituted arylalkyl, and X is substituted or unsubstituted furanyl.

In another embodiment of the compounds of formula IF, L² is —C(O)—, R¹ and R⁵ are independently H or substituted or unsubstituted arylalkyl, and X is substituted or unsubstituted furanyl.

In another embodiment of the compounds of formula IF, L² is a covalent bond, R¹ and R⁵ are both H, and X is substituted or unsubstituted furanyl.

In another embodiment of the compounds of formula IF, L² is —C(R⁴)₂—, R¹ and R⁵ are both H, and X is substituted or unsubstituted furanyl.

In another embodiment of the compounds of formula IF, L² is —CH₂—, R¹ and R⁵ are both H, and X is substituted or unsubstituted furanyl.

In another embodiment of the compounds of formula IF, L² is —CH(CH₃)—, R¹ and R⁵ are both H, and X is substituted or unsubstituted furanyl.

In another embodiment of the compounds of formula IF, L² is —C(O)—, R¹ and R⁵ are both H, and X is substituted or unsubstituted furanyl.

In another embodiment of the compounds of formula IF, L² is a covalent bond, R¹ and R⁵, together with the nitrogen atoms to which they are shown attached, form a 5-membered heterocyclyl ring, and X is substituted or unsubstituted furanyl.

In another embodiment of the compounds of formula IF, L² is —C(R⁴)₂—, R¹ and R⁵, together with the nitrogen atoms to which they are shown attached, form a 5-membered heterocyclyl ring, and X is substituted or unsubstituted furanyl.

In another embodiment of the compounds of formula IF, L² is —CH₂—, R¹ and R⁵, together with the nitrogen atoms to which they are shown attached, form a 5-membered heterocyclyl ring, and X is substituted or unsubstituted furanyl.

In another embodiment of the compounds of formula IF, L² is —CH(CH₃)—, R¹ and R⁵, together with the nitrogen atoms to which they are shown attached, form a 5-membered heterocyclyl ring, and X is substituted or unsubstituted furanyl.

In another embodiment of the compounds of formula IF, L² is —C(O)—, R¹ and R⁵, together with the nitrogen atoms to which they are shown attached, form a 5-membered heterocyclyl ring, and X is substituted or unsubstituted furanyl.

In another embodiment of the compounds of formula IF, L² is a covalent bond, R¹ and R⁵, together with the nitrogen atoms to which they are shown attached, form a 6-membered heterocyclyl ring, and X is substituted or unsubstituted furanyl.

In another embodiment of the compounds of formula IF, L² is —C(R⁴)₂—, R¹ and R⁵, together with the nitrogen atoms to which they are shown attached, form a 6-membered heterocyclyl ring, and X is substituted or unsubstituted furanyl.

In another embodiment of the compounds of formula IF, L² is —CH₂—, R¹ and R⁵, together with the nitrogen atoms to which they are shown attached, form a 6-membered heterocyclyl ring, and X is substituted or unsubstituted furanyl.

In another embodiment of the compounds of formula IF, L² is —CH(CH₃)—, R¹ and R⁵, together with the nitrogen atoms to which they are shown attached, form a 6-membered heterocyclyl ring, and X is substituted or unsubstituted furanyl.

In another embodiment of the compounds of formula IF, L² is —C(O)—, R¹ and R⁵, together with the nitrogen atoms to which they are shown attached, form a 6-membered heterocyclyl ring, and X is substituted or unsubstituted furanyl.

In another embodiment of the compounds of formula IF, L² is a covalent bond, R¹ and R⁵ are independently H or alkyl, and X is substituted or unsubstituted pyrrolyl.

In another embodiment of the compounds of formula IF, L² is —C(R⁴)₂—, R¹ and R⁵ are independently H or alkyl, and X is substituted or unsubstituted pyrrolyl.

In another embodiment of the compounds of formula IF, L² is —CH₂—, R¹ and R⁵ are independently H or alkyl, and X is substituted or unsubstituted pyrrolyl.

In another embodiment of the compounds of formula IF, L² is —CH(CH₃)—, R¹ and R⁵ are independently H or alkyl, and X is substituted or unsubstituted pyrrolyl.

In another embodiment of the compounds of formula IF, L² is —C(O)—, R¹ and R⁵ are independently H or alkyl, and X is substituted or unsubstituted pyrrolyl.

In another embodiment of the compounds of formula IF, L² is a covalent bond, R¹ and R⁵ are independently H or methyl, and X is substituted or unsubstituted pyrrolyl.

In another embodiment of the compounds of formula IF, L² is —C(R⁴)₂—, R¹ and R⁵ are independently H or methyl, and X is substituted or unsubstituted pyrrolyl.

In another embodiment of the compounds of formula IF, L² is —CH₂—, R¹ and R⁵ are independently H or methyl, and X is substituted or unsubstituted pyrrolyl.

In another embodiment of the compounds of formula IF, L² is —CH(CH₃)—, R¹ and R⁵ are independently H or methyl, and X is substituted or unsubstituted pyrrolyl.

In another embodiment of the compounds of formula IF, L² is —C(O)—, R¹ and R⁵ are independently H or methyl, and X is substituted or unsubstituted pyrrolyl.

In another embodiment of the compounds of formula IF, L² is a covalent bond, R¹ and R⁵ are independently H or substituted or unsubstituted arylalkyl, and X is substituted or unsubstituted pyrrolyl.

In another embodiment of the compounds of formula IF, L² is —C(R⁴)₂—, R¹ and R⁵ are independently H or substituted or unsubstituted arylalkyl, and X is substituted or unsubstituted pyrrolyl.

In another embodiment of the compounds of formula IF, L² is —CH₂—, R¹ and R⁵ are independently H or substituted or unsubstituted arylalkyl, and X is substituted or unsubstituted pyrrolyl.

In another embodiment of the compounds of formula IF, L² is —CH(CH₃)—, R¹ and R⁵ are independently H or substituted or unsubstituted arylalkyl, and X is substituted or unsubstituted pyrrolyl.

In another embodiment of the compounds of formula IF, L² is —C(O)—, R¹ and R⁵ are independently H or substituted or unsubstituted arylalkyl, and X is substituted or unsubstituted pyrrolyl.

In another embodiment of the compounds of formula IF, L² is a covalent bond, R¹ and R⁵ are both H, and X is substituted or unsubstituted pyrrolyl.

In another embodiment of the compounds of formula IF, L² is —C(R⁴)₂—, R¹ and R⁵ are both H, and X is substituted or unsubstituted pyrrolyl.

In another embodiment of the compounds of formula IF, L² is —CH₂—, R¹ and R⁵ are both H, and X is substituted or unsubstituted pyrrolyl.

In another embodiment of the compounds of formula IF, L² is —CH(CH₃)—, R¹ and R⁵ are both H, and X is substituted or unsubstituted pyrrolyl.

In another embodiment of the compounds of formula IF, L² is —C(O)—, R¹ and R⁵ are both H, and X is substituted or unsubstituted pyrrolyl.

In another embodiment of the compounds of formula IF, L² is a covalent bond, R¹ and R⁵, together with the nitrogen atoms to which they are shown attached, form a 5-membered heterocyclyl ring, and X is substituted or unsubstituted pyrrolyl.

In another embodiment of the compounds of formula IF, L² is —C(R⁴)₂—, R¹ and R⁵, together with the nitrogen atoms to which they are shown attached, form a 5-membered heterocyclyl ring, and X is substituted or unsubstituted pyrrolyl.

In another embodiment of the compounds of formula IF, L² is —CH₂—, R¹ and R⁵, together with the nitrogen atoms to which they are shown attached, form a 5-membered heterocyclyl ring, and X is substituted or unsubstituted pyrrolyl.

In another embodiment of the compounds of formula IF, L² is —CH(CH₃)—, R¹ and R⁵, together with the nitrogen atoms to which they are shown attached, form a 5-membered heterocyclyl ring, and X is substituted or unsubstituted pyrrolyl.

In another embodiment of the compounds of formula IF, L² is —C(O)—, R¹ and R⁵, together with the nitrogen atoms to which they are shown attached, form a 5-membered heterocyclyl ring, and X is substituted or unsubstituted pyrrolyl.

In another embodiment of the compounds of formula IF, L² is a covalent bond, R¹ and R⁵, together with the nitrogen atoms to which they are shown attached, form a 6-membered heterocyclyl ring, and X is substituted or unsubstituted pyrrolyl.

In another embodiment of the compounds of formula IF, L² is —C(R⁴)₂—, R¹ and R⁵, together with the nitrogen atoms to which they are shown attached, form a 6-membered heterocyclyl ring, and X is substituted or unsubstituted pyrrolyl.

In another embodiment of the compounds of formula IF, L² is —CH₂—, R¹ and R⁵, together with the nitrogen atoms to which they are shown attached, form a 6-membered heterocyclyl ring, and X is substituted or unsubstituted pyrrolyl.

In another embodiment of the compounds of formula IF, L² is —CH(CH₃)—, R¹ and R⁵, together with the nitrogen atoms to which they are shown attached, form a 6-membered heterocyclyl ring, and X is substituted or unsubstituted pyrrolyl.

In another embodiment of the compounds of formula IF, L³ is —C(O)—, R¹ and R⁵, together with the nitrogen atoms to which they are shown attached, form a 6-membered heterocyclyl ring, and X is substituted or unsubstituted pyrrolyl.

In another embodiment of the compounds of formula IF, L³ is —C(R⁴)₂—.

In another embodiment of the compounds of formula IF, L³ is —CH₂—.

In another embodiment of the compounds of formula IF, L³ is —CH(CH₃)—.

In another embodiment of the compounds of formula IF, L³ is —C(O)—.

In another embodiment of the compounds of formula IF, L³ is —S(O)₂—.

In another embodiment of the compounds of formula IF, L⁴ is a covalent bond.

In another embodiment of the compounds of formula IF, L⁴ is —C(R⁴)₂—.

In another embodiment of the compounds of formula IF, L⁴ is —CH₂—.

In another embodiment of the compounds of formula IF, L⁴ is —CH(CH₃)—.

In another embodiment of the compounds of formula IF, L⁴ is —C(O)—.

In another embodiment of the compounds of formula IF, L⁴ is —S(O)₂—.

In another embodiment of the compounds of formula IF, L³ is —C(R⁴)₂—, and L⁴ is a covalent bond.

In another embodiment of the compounds of formula IF, L³ is —CH₂—, and L⁴ is a covalent bond.

In another embodiment of the compounds of formula IF, L³ is —CH(CH₃)—, and L⁴ is a covalent bond.

In another embodiment of the compounds of formula IF, L³ is —C(O)—, and L⁴ is a covalent bond.

In another embodiment of the compounds of formula IF, L³ is —S(O)₂—, and L⁴ is a covalent bond.

In another embodiment of the compounds of formula IF, L³ is —C(R⁴)₂—, and L⁴ is —C(R⁴)₂—.

In another embodiment of the compounds of formula IF, L³ is —CH₂—, and L⁴ is —C(R⁴)₂—.

In another embodiment of the compounds of formula IF, L³ is —CH(CH₃)—, and L⁴ is —C(R⁴)₂—.

In another embodiment of the compounds of formula IF, L³ is —C(O)—, and L⁴ is —C(R⁴)₂—.

In another embodiment of the compounds of formula IF, L³ is —S(O)₂—, and L⁴ is —C(R⁴)₂—.

In another embodiment of the compounds of formula IF, L³ is —C(R⁴)₂—, and L⁴ is —CH₂—.

In another embodiment of the compounds of formula IF, L³ is —CH₂—, and L⁴ is —CH₂—.

In another embodiment of the compounds of formula IF, L³ is —CH(CH₃)—, and L⁴ is —CH₂—.

In another embodiment of the compounds of formula IF, L³ is —C(O)—, and L⁴ is —CH₂—.

In another embodiment of the compounds of formula IF, L³ is —S(O)₂—, and L⁴ is —CH₂—.

In another embodiment of the compounds of formula IF, L³ is —C(R⁴)₂—, and L⁴ is —CH(CH₃)—.

In another embodiment of the compounds of formula IF, L³ is —CH₂—, and L⁴ is —CH(CH₃)—.

In another embodiment of the compounds of formula IF, L³ is —CH(CH₃)—, and L⁴ is —CH(CH₃)—.

In another embodiment of the compounds of formula IF, L³ is —C(O)—, and L⁴ is —CH(CH₃)—.

In another embodiment of the compounds of formula IF, L³ is —S(O)₂—, and L⁴ is —CH(CH₃)—.

In another embodiment of the compounds of formula IF, L³ is —C(R⁴)₂—, and L⁴ is —C(O)—.

In another embodiment of the compounds of formula IF, L³ is —CH₂—, and L⁴ is —C(O)—.

In another embodiment of the compounds of formula IF, L³ is —CH(CH₃)—, and L⁴ is —C(O)—.

In another embodiment of the compounds of formula IF, L³ is —C(O)—, and L⁴ is —C(O)—.

In another embodiment of the compounds of formula IF, L³ is —S(O)₂—, and L⁴ is —C(O)—.

In another embodiment of the compounds of formula IF, L³ is —C(R⁴)₂—, and L⁴ is —S(O)₂—.

In another embodiment of the compounds of formula IF, L³ is —CH₂—, and L⁴ is —S(O)₂—.

In another embodiment of the compounds of formula IF, L³ is —CH(CH₃)—, and L⁴ is —S(O)₂—.

In another embodiment of the compounds of formula IF, L³ is —C(O)—, and L⁴ is —S(O)₂—.

In another embodiment of the compounds of formula IF, L³ is —S(O)₂—, and L⁴ is —S(O)₂—.

In another embodiment of the compounds of formula IF, R⁶ is H.

In another embodiment of the compounds of formula IF, R⁶ is substituted or unsubstituted arylalkyl.

In another embodiment of the compounds of formula IF, R⁶ is substituted or unsubstituted benzyl.

In another embodiment of the compounds of formula IF, R⁶ is substituted or unsubstituted cycloalkyl.

In another embodiment of the compounds of formula IF, R⁶ is substituted or unsubstituted cyclopentyl.

In another embodiment of the compounds of formula IF, R⁶ is substituted or unsubstituted cyclohexyl.

In another embodiment of the compounds of formula IF, R⁶ is substituted or unsubstituted tetrahydronaphthyl.

In another embodiment of the compounds of formula IF, L³ is —C(R⁴)₂—, L⁴ is a covalent bond, and Y is substituted or unsubstituted aryl.

In another embodiment of the compounds of formula IF, L³ is —CH₂—, L⁴ is a covalent bond, and Y is substituted or unsubstituted aryl.

In another embodiment of the compounds of formula IF, L³ is —CH(CH₃)—, L⁴ is a covalent bond, and Y is substituted or unsubstituted aryl.

In another embodiment of the compounds of formula IF, L³ is —C(O)—, L⁴ is a covalent bond, and Y is substituted or unsubstituted aryl.

In another embodiment of the compounds of formula IF, L³ is —S(O)₂—, L⁴ is a covalent bond, and Y is substituted or unsubstituted aryl.

In another embodiment of the compounds of formula IF, L³ is —C(R⁴)₂—, L⁴ is —C(R⁴)₂—, and Y is substituted or unsubstituted aryl.

In another embodiment of the compounds of formula IF, L³ is —CH₂—, L⁴ is —C(R⁴)₂—, and Y is substituted or unsubstituted aryl.

In another embodiment of the compounds of formula IF, L³ is —CH(CH₃)—, L⁴ is —C(R⁴)₂—, and Y is substituted or unsubstituted aryl.

In another embodiment of the compounds of formula IF, L³ is —C(O)—, L⁴ is —C(R⁴)₂—, and Y is substituted or unsubstituted aryl.

In another embodiment of the compounds of formula IF, L³ is —S(O)₂—, L⁴ is —C(R⁴)₂—, and Y is substituted or unsubstituted aryl.

In another embodiment of the compounds of formula IF, L³ is —C(R⁴)₂—, L⁴ is —CH₂—, and Y is substituted or unsubstituted aryl.

In another embodiment of the compounds of formula IF, L³ is —CH₂—, L⁴ is —CH₂—, and Y is substituted or unsubstituted aryl.

In another embodiment of the compounds of formula IF, L³ is —CH(CH₃)—, L⁴ is —CH₂—, and Y is substituted or unsubstituted aryl.

In another embodiment of the compounds of formula IF, L³ is —C(O)—, L⁴ is —CH₂—, and Y is substituted or unsubstituted aryl.

In another embodiment of the compounds of formula IF, L³ is —S(O)₂—, L⁴ is —CH₂—, and Y is substituted or unsubstituted aryl.

In another embodiment of the compounds of formula IF, L³ is —C(R⁴)₂—, L⁴ is —CH(CH₃)—, and Y is substituted or unsubstituted aryl.

In another embodiment of the compounds of formula IF, L³ is —CH₂—, L⁴ is —CH(CH₃)—, and Y is substituted or unsubstituted aryl.

In another embodiment of the compounds of formula IF, L³ is —CH(CH₃)—, L⁴ is —CH(CH₃)—, and Y is substituted or unsubstituted aryl.

In another embodiment of the compounds of formula IF, L³ is —C(O)—, L⁴ is —CH(CH₃)—, and Y is substituted or unsubstituted aryl.

In another embodiment of the compounds of formula IF, L³ is —S(O)₂—, L⁴ is —CH(CH₃)—, and Y is substituted or unsubstituted aryl.

In another embodiment of the compounds of formula IF, L³ is —C(R⁴)₂—, L⁴ is —C(O)—, and Y is substituted or unsubstituted aryl.

In another embodiment of the compounds of formula IF, L³ is —CH₂—, L⁴ is —C(O)—, and Y is substituted or unsubstituted aryl.

In another embodiment of the compounds of formula IF, L³ is —CH(CH₃)—, L⁴ is —C(O)—, and Y is substituted or unsubstituted aryl.

In another embodiment of the compounds of formula IF, L³ is —C(O)—, L⁴ is —C(O)—, and Y is substituted or unsubstituted aryl.

In another embodiment of the compounds of formula IF, L³ is —S(O)₂—, L⁴ is —C(O)—, and Y is substituted or unsubstituted aryl.

In another embodiment of the compounds of formula IF, L³ is —C(R⁴)₂—, L⁴ is —S(O)₂—, and Y is substituted or unsubstituted aryl.

In another embodiment of the compounds of formula IF, L³ is —CH₂—, L⁴ is —S(O)₂—, and Y is substituted or unsubstituted aryl.

In another embodiment of the compounds of formula IF, L³ is —CH(CH₃)—, L⁴ is —S(O)₂—, and Y is substituted or unsubstituted aryl.

In another embodiment of the compounds of formula IF, L³ is —C(O)—, L⁴ is —S(O)₂—, and Y is substituted or unsubstituted aryl.

In another embodiment of the compounds of formula IF, L³ is —S(O)₂—, L⁴ is —S(O)₂—, and Y is substituted or unsubstituted aryl.

In another embodiment of the compounds of formula IF, L³ is —C(R⁴)₂—, L⁴ is a covalent bond, and Y is substituted or unsubstituted phenyl.

In another embodiment of the compounds of formula IF, L³ is —CH₂—, L⁴ is a covalent bond, and Y is substituted or unsubstituted phenyl.

In another embodiment of the compounds of formula IF, L³ is —CH(CH₃)—, L⁴ is a covalent bond, and Y is substituted or unsubstituted phenyl.

In another embodiment of the compounds of formula IF, L³ is —C(O)—, L⁴ is a covalent bond, and Y is substituted or unsubstituted phenyl.

In another embodiment of the compounds of formula IF, L³ is —S(O)₂—, L⁴ is a covalent bond, and Y is substituted or unsubstituted phenyl.

In another embodiment of the compounds of formula IF, L³ is —C(R⁴)₂—, L⁴ is —C(R⁴)₂—, and Y is substituted or unsubstituted phenyl.

In another embodiment of the compounds of formula IF, L³ is —CH₂—, L⁴ is —C(R⁴)₂—, and Y is substituted or unsubstituted phenyl.

In another embodiment of the compounds of formula IF, L³ is —CH(CH₃)—, L⁴ is —C(R⁴)₂—, and Y is substituted or unsubstituted phenyl.

In another embodiment of the compounds of formula IF, L³ is —C(O)—, L⁴ is —C(R⁴)₂—, and Y is substituted or unsubstituted phenyl.

In another embodiment of the compounds of formula IF, L³ is —S(O)₂—, L⁴ is —C(R⁴)₂—, and Y is substituted or unsubstituted phenyl.

In another embodiment of the compounds of formula IF, L³ is —C(R⁴)₂—, L⁴ is —CH₂—, and Y is substituted or unsubstituted phenyl.

In another embodiment of the compounds of formula IF, L³ is —CH₂—, L⁴ is —CH₂—, and Y is substituted or unsubstituted phenyl.

In another embodiment of the compounds of formula IF, L³ is —CH(CH₃)—, L⁴ is —CH₂—, and Y is substituted or unsubstituted phenyl.

In another embodiment of the compounds of formula IF, L³ is —C(O)—, L⁴ is —CH₂—, and Y is substituted or unsubstituted phenyl.

In another embodiment of the compounds of formula IF, L³ is —S(O)₂—, L⁴ is —CH₂—, and Y is substituted or unsubstituted phenyl.

In another embodiment of the compounds of formula IF, L³ is —C(R⁴)₂—, L⁴ is —CH(CH₃)—, and Y is substituted or unsubstituted phenyl.

In another embodiment of the compounds of formula IF, L³ is —CH₂—, L⁴ is —CH(CH₃)—, and Y is substituted or unsubstituted phenyl.

In another embodiment of the compounds of formula IF, L³ is —CH(CH₃)—, L⁴ is —CH(CH₃)—, and Y is substituted or unsubstituted phenyl.

In another embodiment of the compounds of formula IF, L³ is —C(O)—, L⁴ is —CH(CH₃)—, and Y is substituted or unsubstituted phenyl.

In another embodiment of the compounds of formula IF, L³ is —S(O)₂—, L⁴ is —CH(CH₃)—, and Y is substituted or unsubstituted phenyl.

In another embodiment of the compounds of formula IF, L³ is —C(R⁴)₂—, L⁴ is —C(O)—, and Y is substituted or unsubstituted phenyl.

In another embodiment of the compounds of formula IF, L³ is —CH₂—, L⁴ is —C(O)—, and Y is substituted or unsubstituted phenyl.

In another embodiment of the compounds of formula IF, L³ is —CH(CH₃)—, L⁴ is —C(O)—, and Y is substituted or unsubstituted phenyl.

In another embodiment of the compounds of formula IF, L³ is —C(O)—, L⁴ is —C(O)—, and Y is substituted or unsubstituted phenyl.

In another embodiment of the compounds of formula IF, L³ is —S(O)₂—, L⁴ is —C(O)—, and Y is substituted or unsubstituted phenyl.

In another embodiment of the compounds of formula IF, L³ is —C(R⁴)₂—, L⁴ is —S(O)₂—, and Y is substituted or unsubstituted phenyl.

In another embodiment of the compounds of formula IF, L³ is —CH₂—, L⁴ is —S(O)₂—, and Y is substituted or unsubstituted phenyl.

In another embodiment of the compounds of formula IF, L³ is —CH(CH₃)—, L⁴ is —S(O)₂—, and Y is substituted or unsubstituted phenyl.

In another embodiment of the compounds of formula IF, L³ is —C(O)—, L⁴ is —S(O)₂—, and Y is substituted or unsubstituted phenyl.

In another embodiment of the compounds of formula IF, L³ is —S(O)₂—, L⁴ is —S(O)₂—, and Y is substituted or unsubstituted phenyl.

In another embodiment of the compounds of formula IF, L³ is —C(R⁴)₂—, L⁴ is a covalent bond, and Y is substituted or unsubstituted heteroaryl.

In another embodiment of the compounds of formula IF, L³ is —CH₂—, L⁴ is a covalent bond, and Y is substituted or unsubstituted heteroaryl.

In another embodiment of the compounds of formula IF, L³ is —CH(CH₃)—, L⁴ is a covalent bond, and Y is substituted or unsubstituted heteroaryl.

In another embodiment of the compounds of formula IF, L³ is —C(O)—, L⁴ is a covalent bond, and Y is substituted or unsubstituted heteroaryl.

In another embodiment of the compounds of formula IF, L³ is —S(O)₂—, L⁴ is a covalent bond, and Y is substituted or unsubstituted heteroaryl.

In another embodiment of the compounds of formula IF, L³ is —C(R⁴)₂—, L⁴ is —C(R⁴)₂—, and Y is substituted or unsubstituted heteroaryl.

In another embodiment of the compounds of formula IF, L³ is —CH₂—, L⁴ is —C(R⁴)₂—, and Y is substituted or unsubstituted heteroaryl.

In another embodiment of the compounds of formula IF, L³ is —CH(CH₃)—, L⁴ is —C(R⁴)₂—, and Y is substituted or unsubstituted heteroaryl.

In another embodiment of the compounds of formula IF, L³ is —C(O)—, L⁴ is —C(R⁴)₂—, and Y is substituted or unsubstituted heteroaryl.

In another embodiment of the compounds of formula IF, L³ is —S(O)₂—, L⁴ is —C(R⁴)₂—, and Y is substituted or unsubstituted heteroaryl.

In another embodiment of the compounds of formula IF, L³ is —C(R⁴)₂—, L⁴ is —CH₂—, and Y is substituted or unsubstituted heteroaryl.

In another embodiment of the compounds of formula IF, L³ is —CH₂—, L⁴ is —CH₂—, and Y is substituted or unsubstituted heteroaryl.

In another embodiment of the compounds of formula IF, L³ is —CH(CH₃)—, L⁴ is —CH₂—, and Y is substituted or unsubstituted heteroaryl.

In another embodiment of the compounds of formula IF, L³ is —C(O)—, L⁴ is —CH₂—, and Y is substituted or unsubstituted heteroaryl.

In another embodiment of the compounds of formula IF, L³ is —S(O)₂—, L⁴ is —CH₂—, and Y is substituted or unsubstituted heteroaryl.

In another embodiment of the compounds of formula IF, L³ is —C(R⁴)₂—, L⁴ is —CH(CH₃)—, and Y is substituted or unsubstituted heteroaryl.

In another embodiment of the compounds of formula IF, L³ is —CH₂—, L⁴ is —CH(CH₃)—, and Y is substituted or unsubstituted heteroaryl.

In another embodiment of the compounds of formula IF, L³ is —CH(CH₃)—, L⁴ is —CH(CH₃)—, and Y is substituted or unsubstituted heteroaryl.

In another embodiment of the compounds of formula IF, L³ is —C(O)—, L⁴ is —CH(CH₃)—, and Y is substituted or unsubstituted heteroaryl.

In another embodiment of the compounds of formula IF, L³ is —S(O)₂—, L⁴ is —CH(CH₃)—, and Y is substituted or unsubstituted heteroaryl.

In another embodiment of the compounds of formula IF, L³ is —C(R⁴)₂—, L⁴ is —C(O)—, and Y is substituted or unsubstituted heteroaryl.

In another embodiment of the compounds of formula IF, L³ is —CH₂—, L⁴ is —C(O)—, and Y is substituted or unsubstituted heteroaryl.

In another embodiment of the compounds of formula IF, L³ is —CH(CH₃)—, L⁴ is —C(O)—, and Y is substituted or unsubstituted heteroaryl.

In another embodiment of the compounds of formula IF, L³ is —C(O)—, L⁴ is —C(O)—, and Y is substituted or unsubstituted heteroaryl.

In another embodiment of the compounds of formula IF, L³ is —S(O)₂—, L⁴ is —C(O)—, and Y is substituted or unsubstituted heteroaryl.

In another embodiment of the compounds of formula IF, L³ is —C(R⁴)₂—, L⁴ is —S(O)₂—, and Y is substituted or unsubstituted heteroaryl.

In another embodiment of the compounds of formula IF, L³ is —CH₂—, L⁴ is —S(O)₂—, and Y is substituted or unsubstituted heteroaryl.

In another embodiment of the compounds of formula IF, L³ is —CH(CH₃)—, L⁴ is —S(O)₂—, and Y is substituted or unsubstituted heteroaryl.

In another embodiment of the compounds of formula IF, L³ is —C(O)—, L⁴ is —S(O)₂—, and Y is substituted or unsubstituted heteroaryl.

In another embodiment of the compounds of formula IF, L³ is —S(O)₂—, L⁴ is —S(O)₂—, and Y is substituted or unsubstituted heteroaryl.

In another embodiment of the compounds of formula IF, L³ is —C(R⁴)₂—, L⁴ is a covalent bond, and Y is substituted or unsubstituted pyridyl.

In another embodiment of the compounds of formula IF, L³ is —CH₂—, L⁴ is a covalent bond, and Y is substituted or unsubstituted pyridyl.

In another embodiment of the compounds of formula IF, L³ is —CH(CH₃)—, L⁴ is a covalent bond, and Y is substituted or unsubstituted pyridyl.

In another embodiment of the compounds of formula IF, L³ is —C(O)—, L⁴ is a covalent bond, and Y is substituted or unsubstituted pyridyl.

In another embodiment of the compounds of formula IF, L³ is —S(O)₂—, L⁴ is a covalent bond, and Y is substituted or unsubstituted pyridyl.

In another embodiment of the compounds of formula IF, L³ is —C(R⁴)₂—, L⁴ is —C(R⁴)₂—, and Y is substituted or unsubstituted pyridyl.

In another embodiment of the compounds of formula IF, L³ is —CH₂—, L⁴ is —C(R⁴)₂—, and Y is substituted or unsubstituted pyridyl.

In another embodiment of the compounds of formula IF, L³ is —CH(CH₃)—, L⁴ is —C(R⁴)₂—, and Y is substituted or unsubstituted pyridyl.

In another embodiment of the compounds of formula IF, L³ is —C(O)—, L⁴ is —C(R⁴)₂—, and Y is substituted or unsubstituted pyridyl.

In another embodiment of the compounds of formula IF, L³ is —S(O)₂—, L⁴ is —C(R⁴)₂—, and Y is substituted or unsubstituted pyridyl.

In another embodiment of the compounds of formula IF, L³ is —C(R⁴)₂—, L⁴ is —CH₂—, and Y is substituted or unsubstituted pyridyl.

In another embodiment of the compounds of formula IF, L³ is —CH₂—, L⁴ is —CH₂—, and Y is substituted or unsubstituted pyridyl.

In another embodiment of the compounds of formula IF, L³ is —CH(CH₃)—, L⁴ is —CH₂—, and Y is substituted or unsubstituted pyridyl.

In another embodiment of the compounds of formula IF, L³ is —C(O)—, L⁴ is —CH₂—, and Y is substituted or unsubstituted pyridyl.

In another embodiment of the compounds of formula IF, L³ is —S(O)₂—, L⁴ is —CH₂—, and Y is substituted or unsubstituted pyridyl.

In another embodiment of the compounds of formula IF, L³ is —C(R⁴)₂—, L⁴ is —CH(CH₃)—, and Y is substituted or unsubstituted pyridyl.

In another embodiment of the compounds of formula IF, L³ is —CH₂—, L⁴ is —CH(CH₃)—, and Y is substituted or unsubstituted pyridyl.

In another embodiment of the compounds of formula IF, L³ is —CH(CH₃)—, L⁴ is —CH(CH₃)—, and Y is substituted or unsubstituted pyridyl.

In another embodiment of the compounds of formula IF, L³ is —C(O)—, L⁴ is —CH(CH₃)—, and Y is substituted or unsubstituted pyridyl.

In another embodiment of the compounds of formula IF, L³ is —S(O)₂—, L⁴ is —CH(CH₃)—, and Y is substituted or unsubstituted pyridyl.

In another embodiment of the compounds of formula IF, L³ is —C(R⁴)₂—, L⁴ is —C(O)—, and Y is substituted or unsubstituted pyridyl.

In another embodiment of the compounds of formula IF, L³ is —CH₂—, L⁴ is —C(O)—, and Y is substituted or unsubstituted pyridyl.

In another embodiment of the compounds of formula IF, L³ is —CH(CH₃)—, L⁴ is —C(O)—, and Y is substituted or unsubstituted pyridyl.

In another embodiment of the compounds of formula IF, L³ is —C(O)—, L⁴ is —C(O)—, and Y is substituted or unsubstituted pyridyl.

In another embodiment of the compounds of formula IF, L³ is —S(O)₂—, L⁴ is —C(O)—, and Y is substituted or unsubstituted pyridyl.

In another embodiment of the compounds of formula IF, L³ is —C(R⁴)₂—, L⁴ is —S(O)₂—, and Y is substituted or unsubstituted pyridyl.

In another embodiment of the compounds of formula IF, L³ is —CH₂—, L⁴ is —S(O)₂—, and Y is substituted or unsubstituted pyridyl.

In another embodiment of the compounds of formula IF, L³ is —CH(CH₃)—, L⁴ is —S(O)₂—, and Y is substituted or unsubstituted pyridyl.

In another embodiment of the compounds of formula IF, L³ is —C(O)—, L⁴ is —S(O)₂—, and Y is substituted or unsubstituted pyridyl.

In another embodiment of the compounds of formula IF, L³ is —S(O)₂—, L⁴ is —S(O)₂—, and Y is substituted or unsubstituted pyridyl.

In another embodiment of the compounds of formula IF, L³ is —C(R⁴)₂—, L⁴ is a covalent bond, and Y is substituted or unsubstituted pyrimidyl.

In another embodiment of the compounds of formula IF, L³ is —CH₂—, L⁴ is a covalent bond, and Y is substituted or unsubstituted pyrimidyl.

In another embodiment of the compounds of formula IF, L³ is —CH(CH₃)—, L⁴ is a covalent bond, and Y is substituted or unsubstituted pyrimidyl.

In another embodiment of the compounds of formula IF, L³ is —C(O)—, L⁴ is a covalent bond, and Y is substituted or unsubstituted pyrimidyl.

In another embodiment of the compounds of formula IF, L³ is —S(O)₂—, L⁴ is a covalent bond, and Y is substituted or unsubstituted pyrimidyl.

In another embodiment of the compounds of formula IF, L³ is —C(R⁴)₂—, L⁴ is —C(R⁴)₂—, and Y is substituted or unsubstituted pyrimidyl.

In another embodiment of the compounds of formula IF, L³ is —CH₂—, L⁴ is —C(R⁴)₂—, and Y is substituted or unsubstituted pyrimidyl.

In another embodiment of the compounds of formula IF, L³ is —CH(CH₃)—, L⁴ is —C(R⁴)₂—, and Y is substituted or unsubstituted pyrimidyl.

In another embodiment of the compounds of formula IF, L³ is —C(O)—, L⁴ is —C(R⁴)₂—, and Y is substituted or unsubstituted pyrimidyl.

In another embodiment of the compounds of formula IF, L³ is —S(O)₂—, L⁴ is —C(R⁴)₂—, and Y is substituted or unsubstituted pyrimidyl.

In another embodiment of the compounds of formula IF, L³ is —C(R⁴)₂—, L⁴ is —CH₂—, and Y is substituted or unsubstituted pyrimidyl.

In another embodiment of the compounds of formula IF, L³ is —CH₂—, L⁴ is —CH₂—, and Y is substituted or unsubstituted pyrimidyl.

In another embodiment of the compounds of formula IF, L³ is —CH(CH₃)—, L⁴ is —CH₂—, and Y is substituted or unsubstituted pyrimidyl.

In another embodiment of the compounds of formula IF, L³ is —C(O)—, L⁴ is —CH₂—, and Y is substituted or unsubstituted pyrimidyl.

In another embodiment of the compounds of formula IF, L³ is —S(O)₂—, L⁴ is —CH₂—, and Y is substituted or unsubstituted pyrimidyl.

In another embodiment of the compounds of formula IF, L³ is —C(R⁴)₂—, L⁴ is —CH(CH₃)—, and Y is substituted or unsubstituted pyrimidyl.

In another embodiment of the compounds of formula IF, L³ is —CH₂—, L⁴ is —CH(CH₃)—, and Y is substituted or unsubstituted pyrimidyl.

In another embodiment of the compounds of formula IF, L³ is —CH(CH₃)—, L⁴ is —CH(CH₃)—, and Y is substituted or unsubstituted pyrimidyl.

In another embodiment of the compounds of formula IF, L³ is —C(O)—, L⁴ is —CH(CH₃)—, and Y is substituted or unsubstituted pyrimidyl.

In another embodiment of the compounds of formula IF, L³ is —S(O)₂—, L⁴ is —CH(CH₃)—, and Y is substituted or unsubstituted pyrimidyl.

In another embodiment of the compounds of formula IF, L³ is —C(R⁴)₂—, L⁴ is —C(O)—, and Y is substituted or unsubstituted pyrimidyl.

In another embodiment of the compounds of formula IF, L³ is —CH₂—, L⁴ is —C(O)—, and Y is substituted or unsubstituted pyrimidyl.

In another embodiment of the compounds of formula IF, L³ is —CH(CH₃)—, L⁴ is —C(O)—, and Y is substituted or unsubstituted pyrimidyl.

In another embodiment of the compounds of formula IF, L³ is —C(O)—, L⁴ is —C(O)—, and Y is substituted or unsubstituted pyrimidyl.

In another embodiment of the compounds of formula IF, L³ is —S(O)₂—, L⁴ is —C(O)—, and Y is substituted or unsubstituted pyrimidyl.

In another embodiment of the compounds of formula IF, L³ is —C(R⁴)₂—, L⁴ is —S(O)₂—, and Y is substituted or unsubstituted pyrimidyl.

In another embodiment of the compounds of formula IF, L³ is —CH₂—, L⁴ is —S(O)₂—, and Y is substituted or unsubstituted pyrimidyl.

In another embodiment of the compounds of formula IF, L³ is —CH(CH₃)—, L⁴ is —S(O)₂—, and Y is substituted or unsubstituted pyrimidyl.

In another embodiment of the compounds of formula IF, L³ is —C(O)—, L⁴ is —S(O)₂—, and Y is substituted or unsubstituted pyrimidyl.

In another embodiment of the compounds of formula IF, L³ is —S(O)₂—, L⁴ is —S(O)₂—, and Y is substituted or unsubstituted pyrimidyl.

In another embodiment of the compounds of formula IF, L³ is —C(R⁴)₂—, L⁴ is a covalent bond, and Y is substituted or unsubstituted indolyl.

In another embodiment of the compounds of formula IF, L³ is —CH₂—, L⁴ is a covalent bond, and Y is substituted or unsubstituted indolyl.

In another embodiment of the compounds of formula IF, L³ is —CH(CH₃)—, L⁴ is a covalent bond, and Y is substituted or unsubstituted indolyl.

In another embodiment of the compounds of formula IF, L³ is —C(O)—, L⁴ is a covalent bond, and Y is substituted or unsubstituted indolyl.

In another embodiment of the compounds of formula IF, L³ is —S(O)₂—, L⁴ is a covalent bond, and Y is substituted or unsubstituted indolyl.

In another embodiment of the compounds of formula IF, L³ is —C(R⁴)₂—, L⁴ is —C(R⁴)₂—, and Y is substituted or unsubstituted indolyl.

In another embodiment of the compounds of formula IF, L³ is —CH₂—, L⁴ is —C(R⁴)₂—, and Y is substituted or unsubstituted indolyl.

In another embodiment of the compounds of formula IF, L³ is —CH(CH₃)—, L⁴ is —C(R⁴)₂—, and Y is substituted or unsubstituted indolyl.

In another embodiment of the compounds of formula IF, L³ is —C(O)—, L⁴ is —C(R⁴)₂—, and Y is substituted or unsubstituted indolyl.

In another embodiment of the compounds of formula IF, L³ is —S(O)₂—, L⁴ is —C(R⁴)₂—, and Y is substituted or unsubstituted indolyl.

In another embodiment of the compounds of formula IF, L³ is —C(R⁴)₂—, L⁴ is —CH₂—, and Y is substituted or unsubstituted indolyl.

In another embodiment of the compounds of formula IF, L³ is —CH₂—, L⁴ is —CH₂—, and Y is substituted or unsubstituted indolyl.

In another embodiment of the compounds of formula IF, L³ is —CH(CH₃)—, L⁴ is —CH₂—, and Y is substituted or unsubstituted indolyl.

In another embodiment of the compounds of formula IF, L³ is —C(O)—, L⁴ is —CH₂—, and Y is substituted or unsubstituted indolyl.

In another embodiment of the compounds of formula IF, L³ is —S(O)₂—, L⁴ is —CH₂—, and Y is substituted or unsubstituted indolyl.

In another embodiment of the compounds of formula IF, L³ is —C(R⁴)₂—, L⁴ is —CH(CH₃)—, and Y is substituted or unsubstituted indolyl.

In another embodiment of the compounds of formula IF, L³ is —CH₂—, L⁴ is —CH(CH₃)—, and Y is substituted or unsubstituted indolyl.

In another embodiment of the compounds of formula IF, L³ is —CH(CH₃)—, L⁴ is —CH(CH₃)—, and Y is substituted or unsubstituted indolyl.

In another embodiment of the compounds of formula IF, L³ is —C(O)—, L⁴ is —CH(CH₃)—, and Y is substituted or unsubstituted indolyl.

In another embodiment of the compounds of formula IF, L³ is —S(O)₂—, L⁴ is —CH(CH₃)—, and Y is substituted or unsubstituted indolyl.

In another embodiment of the compounds of formula IF, L³ is —C(R⁴)₂—, L⁴ is —C(O)—, and Y is substituted or unsubstituted indolyl.

In another embodiment of the compounds of formula IF, L³ is —CH₂—, L⁴ is —C(O)—, and Y is substituted or unsubstituted indolyl.

In another embodiment of the compounds of formula IF, L³ is —CH(CH₃)—, L⁴ is —C(O)—, and Y is substituted or unsubstituted indolyl.

In another embodiment of the compounds of formula IF, L³ is —C(O)—, L⁴ is —C(O)—, and Y is substituted or unsubstituted indolyl.

In another embodiment of the compounds of formula IF, L³ is —S(O)₂—, L⁴ is —C(O)—, and Y is substituted or unsubstituted indolyl.

In another embodiment of the compounds of formula IF, L³ is —C(R⁴)₂—, L⁴ is —S(O)₂—, and Y is substituted or unsubstituted indolyl.

In another embodiment of the compounds of formula IF, L³ is —CH₂—, L⁴ is —S(O)₂—, and Y is substituted or unsubstituted indolyl.

In another embodiment of the compounds of formula IF, L³ is —CH(CH₃)—, L⁴ is —S(O)₂—, and Y is substituted or unsubstituted indolyl.

In another embodiment of the compounds of formula IF, L³ is —C(O)—, L⁴ is —S(O)₂—, and Y is substituted or unsubstituted indolyl.

In another embodiment of the compounds of formula IF, L³ is —S(O)₂—, L⁴ is —S(O)₂—, and Y is substituted or unsubstituted indolyl.

In another embodiment of the compounds of formula IF, L³ is —C(R⁴)₂—, L⁴ is a covalent bond, and Y is substituted or unsubstituted benzimidazolyl.

In another embodiment of the compounds of formula IF, L³ is —CH₂—, L⁴ is a covalent bond, and Y is substituted or unsubstituted benzimidazolyl.

In another embodiment of the compounds of formula IF, L³ is —CH(CH₃)—, L⁴ is a covalent bond, and Y is substituted or unsubstituted benzimidazolyl.

In another embodiment of the compounds of formula IF, L³ is —C(O)—, L⁴ is a covalent bond, and Y is substituted or unsubstituted benzimidazolyl.

In another embodiment of the compounds of formula IF, L³ is —S(O)₂—, L⁴ is a covalent bond, and Y is substituted or unsubstituted benzimidazolyl.

In another embodiment of the compounds of formula IF, L³ is —C(R⁴)₂—, L⁴ is —C(R⁴)₂—, and Y is substituted or unsubstituted benzimidazolyl.

In another embodiment of the compounds of formula IF, L³ is —CH₂—, L⁴ is —C(R⁴)₂—, and Y is substituted or unsubstituted benzimidazolyl.

In another embodiment of the compounds of formula IF, L³ is —CH(CH₃)—, L⁴ is —C(R⁴)₂—, and Y is substituted or unsubstituted benzimidazolyl.

In another embodiment of the compounds of formula IF, L³ is —C(O)—, L⁴ is —C(R⁴)₂—, and Y is substituted or unsubstituted benzimidazolyl.

In another embodiment of the compounds of formula IF, L³ is —S(O)₂—, L⁴ is —C(R⁴)₂—, and Y is substituted or unsubstituted benzimidazolyl.

In another embodiment of the compounds of formula IF, L³ is —C(R⁴)₂—, L⁴ is —CH₂—, and Y is substituted or unsubstituted benzimidazolyl.

In another embodiment of the compounds of formula IF, L³ is —CH₂—, L⁴ is —CH₂—, and Y is substituted or unsubstituted benzimidazolyl.

In another embodiment of the compounds of formula IF, L³ is —CH(CH₃)—, L⁴ is —CH₂—, and Y is substituted or unsubstituted benzimidazolyl.

In another embodiment of the compounds of formula IF, L³ is —C(O)—, L⁴ is —CH₂—, and Y is substituted or unsubstituted benzimidazolyl.

In another embodiment of the compounds of formula IF, L³ is —S(O)₂—, L⁴ is —CH₂—, and Y is substituted or unsubstituted benzimidazolyl.

In another embodiment of the compounds of formula IF, L³ is —C(R⁴)₂—, L⁴ is —CH(CH₃)—, and Y is substituted or unsubstituted benzimidazolyl.

In another embodiment of the compounds of formula IF, L³ is —CH₂—, L⁴ is —CH(CH₃)—, and Y is substituted or unsubstituted benzimidazolyl.

In another embodiment of the compounds of formula IF, L³ is —CH(CH₃)—, L⁴ is —CH(CH₃)—, and Y is substituted or unsubstituted benzimidazolyl.

In another embodiment of the compounds of formula IF, L³ is —C(O)—, L⁴ is —CH(CH₃)—, and Y is substituted or unsubstituted benzimidazolyl.

In another embodiment of the compounds of formula IF, L³ is —S(O)₂—, L⁴ is —CH(CH₃)—, and Y is substituted or unsubstituted benzimidazolyl.

In another embodiment of the compounds of formula IF, L³ is —C(R⁴)₂—, L⁴ is —C(O)—, and Y is substituted or unsubstituted benzimidazolyl.

In another embodiment of the compounds of formula IF, L³ is —CH₂—, L⁴ is —C(O)—, and Y is substituted or unsubstituted benzimidazolyl.

In another embodiment of the compounds of formula IF, L³ is —CH(CH₃)—, L⁴ is —C(O)—, and Y is substituted or unsubstituted benzimidazolyl.

In another embodiment of the compounds of formula IF, L³ is —C(O)—, L⁴ is —C(O)—, and Y is substituted or unsubstituted benzimidazolyl.

In another embodiment of the compounds of formula IF, L³ is —S(O)₂—, L⁴ is —C(O)—, and Y is substituted or unsubstituted benzimidazolyl.

In another embodiment of the compounds of formula IF, L³ is —C(R⁴)₂—, L⁴ is —S(O)₂—, and Y is substituted or unsubstituted benzimidazolyl.

In another embodiment of the compounds of formula IF, L³ is —CH₂—, L⁴ is —S(O)₂—, and Y is substituted or unsubstituted benzimidazolyl.

In another embodiment of the compounds of formula IF, L³ is —CH(CH₃)—, L⁴ is —S(O)₂—, and Y is substituted or unsubstituted benzimidazolyl.

In another embodiment of the compounds of formula IF, L³ is —C(O)—, L⁴ is —S(O)₂—, and Y is substituted or unsubstituted benzimidazolyl.

In another embodiment of the compounds of formula IF, L³ is —S(O)₂—, L⁴ is —S(O)₂—, and Y is substituted or unsubstituted benzimidazolyl.

In another embodiment of the compounds of formula IF, L³ is —C(R⁴)₂—, L⁴ is a covalent bond, and Y is substituted or unsubstituted purinyl.

In another embodiment of the compounds of formula IF, L³ is —CH₂—, L⁴ is a covalent bond, and Y is substituted or unsubstituted purinyl.

In another embodiment of the compounds of formula IF, L³ is —CH(CH₃)—, L⁴ is a covalent bond, and Y is substituted or unsubstituted purinyl.

In another embodiment of the compounds of formula IF, L³ is —C(O)—, L⁴ is a covalent bond, and Y is substituted or unsubstituted purinyl.

In another embodiment of the compounds of formula IF, L³ is —S(O)₂—, L⁴ is a covalent bond, and Y is substituted or unsubstituted purinyl.

In another embodiment of the compounds of formula IF, L³ is —C(R⁴)₂—, L⁴ is —C(R⁴)₂—, and Y is substituted or unsubstituted purinyl.

In another embodiment of the compounds of formula IF, L³ is —CH₂—, L⁴ is —C(R⁴)₂—, and Y is substituted or unsubstituted purinyl.

In another embodiment of the compounds of formula IF, L³ is —CH(CH₃)—, L⁴ is —C(R⁴)₂—, and Y is substituted or unsubstituted purinyl.

In another embodiment of the compounds of formula IF, L³ is —C(O)—, L⁴ is —C(R⁴)₂—, and Y is substituted or unsubstituted purinyl.

In another embodiment of the compounds of formula IF, L³ is —S(O)₂—, L⁴ is —C(R⁴)₂—, and Y is substituted or unsubstituted purinyl.

In another embodiment of the compounds of formula IF, L³ is —C(R⁴)₂—, L⁴ is —CH₂—, and Y is substituted or unsubstituted purinyl.

In another embodiment of the compounds of formula IF, L³ is —CH₂—, L⁴ is —CH₂—, and Y is substituted or unsubstituted purinyl.

In another embodiment of the compounds of formula IF, L³ is —CH(CH₃)—, L⁴ is —CH₂—, and Y is substituted or unsubstituted purinyl.

In another embodiment of the compounds of formula IF, L³ is —C(O)—, L⁴ is —CH₂—, and Y is substituted or unsubstituted purinyl.

In another embodiment of the compounds of formula IF, L³ is —S(O)₂—, L⁴ is —CH₂—, and Y is substituted or unsubstituted purinyl.

In another embodiment of the compounds of formula IF, L³ is —C(R⁴)₂—, L⁴ is —CH(CH₃)—, and Y is substituted or unsubstituted purinyl.

In another embodiment of the compounds of formula IF, L³ is —CH₂—, L⁴ is —CH(CH₃)—, and Y is substituted or unsubstituted purinyl.

In another embodiment of the compounds of formula IF, L³ is —CH(CH₃)—, L⁴ is —CH(CH₃)—, and Y is substituted or unsubstituted purinyl.

In another embodiment of the compounds of formula IF, L³ is —C(O)—, L⁴ is —CH(CH₃)—, and Y is substituted or unsubstituted purinyl.

In another embodiment of the compounds of formula IF, L³ is —S(O)₂—, L⁴ is —CH(CH₃)—, and Y is substituted or unsubstituted purinyl.

In another embodiment of the compounds of formula IF, L³ is —C(R⁴)₂—, L⁴ is —C(O)—, and Y is substituted or unsubstituted purinyl.

In another embodiment of the compounds of formula IF, L³ is —CH₂—, L⁴ is —C(O)—, and Y is substituted or unsubstituted purinyl.

In another embodiment of the compounds of formula IF, L³ is —CH(CH₃)—, L⁴ is —C(O)—, and Y is substituted or unsubstituted purinyl.

In another embodiment of the compounds of formula IF, L³ is —C(O)—, L⁴ is —C(O)—, and Y is substituted or unsubstituted purinyl.

In another embodiment of the compounds of formula IF, L³ is —S(O)₂—, L⁴ is —C(O)—, and Y is substituted or unsubstituted purinyl.

In another embodiment of the compounds of formula IF, L³ is —C(R⁴)₂—, L⁴ is —S(O)₂—, and Y is substituted or unsubstituted purinyl.

In another embodiment of the compounds of formula IF, L³ is —CH₂—, L⁴ is —S(O)₂—, and Y is substituted or unsubstituted purinyl.

In another embodiment of the compounds of formula IF, L³ is —CH(CH₃)—, L⁴ is —S(O)₂—, and Y is substituted or unsubstituted purinyl.

In another embodiment of the compounds of formula IF, L³ is —C(O)—, L⁴ is —S(O)₂—, and Y is substituted or unsubstituted purinyl.

In another embodiment of the compounds of formula IF, L³ is —S(O)₂—, L⁴ is —S(O)₂—, and Y is substituted or unsubstituted purinyl.

In another embodiment of the compounds of formula IF, L³ is —C(R⁴)₂—, L⁴ is a covalent bond, and Y is substituted or unsubstituted thienyl.

In another embodiment of the compounds of formula IF, L³ is —CH₂—, L⁴ is a covalent bond, and Y is substituted or unsubstituted thienyl.

In another embodiment of the compounds of formula IF, L³ is —CH(CH₃)—, L⁴ is a covalent bond, and Y is substituted or unsubstituted thienyl.

In another embodiment of the compounds of formula IF, L³ is —C(O)—, L⁴ is a covalent bond, and Y is substituted or unsubstituted thienyl.

In another embodiment of the compounds of formula IF, L³ is —S(O)₂—, L⁴ is a covalent bond, and Y is substituted or unsubstituted thienyl.

In another embodiment of the compounds of formula IF, L³ is —C(R⁴)₂—, L⁴ is —C(R⁴)₂—, and Y is substituted or unsubstituted thienyl.

In another embodiment of the compounds of formula IF, L³ is —CH₂—, L⁴ is —C(R⁴)₂—, and Y is substituted or unsubstituted thienyl.

In another embodiment of the compounds of formula IF, L³ is —CH(CH₃)—, L⁴ is —C(R⁴)₂—, and Y is substituted or unsubstituted thienyl.

In another embodiment of the compounds of formula IF, L³ is —C(O)—, L⁴ is —C(R⁴)₂—, and Y is substituted or unsubstituted thienyl.

In another embodiment of the compounds of formula IF, L³ is —S(O)₂—, L⁴ is —C(R⁴)₂—, and Y is substituted or unsubstituted thienyl.

In another embodiment of the compounds of formula IF, L³ is —C(R⁴)₂—, L⁴ is —CH₂—, and Y is substituted or unsubstituted thienyl.

In another embodiment of the compounds of formula IF, L³ is —CH₂—, L⁴ is —CH₂—, and Y is substituted or unsubstituted thienyl.

In another embodiment of the compounds of formula IF, L³ is —CH(CH₃)—, L⁴ is —CH₂—, and Y is substituted or unsubstituted thienyl.

In another embodiment of the compounds of formula IF, L³ is —C(O)—, L⁴ is —CH₂—, and Y is substituted or unsubstituted thienyl.

In another embodiment of the compounds of formula IF, L³ is —S(O)₂—, L⁴ is —CH₂—, and Y is substituted or unsubstituted thienyl.

In another embodiment of the compounds of formula IF, L³ is —C(R⁴)₂—, L⁴ is —CH(CH₃)—, and Y is substituted or unsubstituted thienyl.

In another embodiment of the compounds of formula IF, L³ is —CH₂—, L⁴ is —CH(CH₃)—, and Y is substituted or unsubstituted thienyl.

In another embodiment of the compounds of formula IF, L³ is —CH(CH₃)—, L⁴ is —CH(CH₃)—, and Y is substituted or unsubstituted thienyl.

In another embodiment of the compounds of formula IF, L³ is —C(O)—, L⁴ is —CH(CH₃)—, and Y is substituted or unsubstituted thienyl.

In another embodiment of the compounds of formula IF, L³ is —S(O)₂—, L⁴ is —CH(CH₃)—, and Y is substituted or unsubstituted thienyl.

In another embodiment of the compounds of formula IF, L³ is —C(R⁴)₂—, L⁴ is —C(O)—, and Y is substituted or unsubstituted thienyl.

In another embodiment of the compounds of formula IF, L³ is —CH₂—, L⁴ is —C(O)—, and Y is substituted or unsubstituted thienyl.

In another embodiment of the compounds of formula IF, L³ is —CH(CH₃)—, L⁴ is —C(O)—, and Y is substituted or unsubstituted thienyl.

In another embodiment of the compounds of formula IF, L³ is —C(O)—, L⁴ is —C(O)—, and Y is substituted or unsubstituted thienyl.

In another embodiment of the compounds of formula IF, L³ is —S(O)₂—, L⁴ is —C(O)—, and Y is substituted or unsubstituted thienyl.

In another embodiment of the compounds of formula IF, L³ is —C(R⁴)₂—, L⁴ is —S(O)₂—, and Y is substituted or unsubstituted thienyl.

In another embodiment of the compounds of formula IF, L³ is —CH₂—, L⁴ is —S(O)₂—, and Y is substituted or unsubstituted thienyl.

In another embodiment of the compounds of formula IF, L³ is —CH(CH₃)—, L⁴ is —S(O)₂—, and Y is substituted or unsubstituted thienyl.

In another embodiment of the compounds of formula IF, L³ is —C(O)—, L⁴ is —S(O)₂—, and Y is substituted or unsubstituted thienyl.

In another embodiment of the compounds of formula IF, L³ is —S(O)₂—, L⁴ is —S(O)₂—, and Y is substituted or unsubstituted thienyl.

In another embodiment of the compounds of formula IF, L³ is —C(R⁴)₂—, L⁴ is a covalent bond, and Y is substituted or unsubstituted furanyl.

In another embodiment of the compounds of formula IF, L³ is —CH₂—, L⁴ is a covalent bond, and Y is substituted or unsubstituted furanyl.

In another embodiment of the compounds of formula IF, L³ is —CH(CH₃)—, L⁴ is a covalent bond, and Y is substituted or unsubstituted furanyl.

In another embodiment of the compounds of formula IF, L³ is —C(O)—, L⁴ is a covalent bond, and Y is substituted or unsubstituted furanyl.

In another embodiment of the compounds of formula IF, L³ is —S(O)₂—, L⁴ is a covalent bond, and Y is substituted or unsubstituted furanyl.

In another embodiment of the compounds of formula IF, L³ is —C(R⁴)₂—, L⁴ is —C(R⁴)₂—, and Y is substituted or unsubstituted furanyl.

In another embodiment of the compounds of formula IF, L³ is —CH₂—, L⁴ is —C(R⁴)₂—, and Y is substituted or unsubstituted furanyl.

In another embodiment of the compounds of formula IF, L³ is —CH(CH₃)—, L⁴ is —C(R⁴)₂—, and Y is substituted or unsubstituted furanyl.

In another embodiment of the compounds of formula IF, L³ is —C(O)—, L⁴ is —C(R⁴)₂—, and Y is substituted or unsubstituted furanyl.

In another embodiment of the compounds of formula IF, L³ is —S(O)₂—, L⁴ is —C(R⁴)₂—, and Y is substituted or unsubstituted furanyl.

In another embodiment of the compounds of formula IF, L³ is —C(R⁴)₂—, L⁴ is —CH₂—, and Y is substituted or unsubstituted furanyl.

In another embodiment of the compounds of formula IF, L³ is —CH₂—, L⁴ is —CH₂—, and Y is substituted or unsubstituted furanyl.

In another embodiment of the compounds of formula IF, L³ is —CH(CH₃)—, L⁴ is —CH₂—, and Y is substituted or unsubstituted furanyl.

In another embodiment of the compounds of formula IF, L³ is —C(O)—, L⁴ is —CH₂—, and Y is substituted or unsubstituted furanyl.

In another embodiment of the compounds of formula IF, L³ is —S(O)₂—, L⁴ is —CH₂—, and Y is substituted or unsubstituted furanyl.

In another embodiment of the compounds of formula IF, L³ is —C(R⁴)₂—, L⁴ is —CH(CH₃)—, and Y is substituted or unsubstituted furanyl.

In another embodiment of the compounds of formula IF, L³ is —CH₂—, L⁴ is —CH(CH₃)—, and Y is substituted or unsubstituted furanyl.

In another embodiment of the compounds of formula IF, L³ is —CH(CH₃)—, L⁴ is —CH(CH₃)—, and Y is substituted or unsubstituted furanyl.

In another embodiment of the compounds of formula IF, L³ is —C(O)—, L⁴ is —CH(CH₃)—, and Y is substituted or unsubstituted furanyl.

In another embodiment of the compounds of formula IF, L³ is —S(O)₂—, L⁴ is —CH(CH₃)—, and Y is substituted or unsubstituted furanyl.

In another embodiment of the compounds of formula IF, L³ is —C(R⁴)₂—, L⁴ is —C(O)—, and Y is substituted or unsubstituted furanyl.

In another embodiment of the compounds of formula IF, L³ is —CH₂—, L⁴ is —C(O)—, and Y is substituted or unsubstituted furanyl.

In another embodiment of the compounds of formula IF, L³ is —CH(CH₃)—, L⁴ is —C(O)—, and Y is substituted or unsubstituted furanyl.

In another embodiment of the compounds of formula IF, L³ is —C(O)—, L⁴ is —C(O)—, and Y is substituted or unsubstituted furanyl.

In another embodiment of the compounds of formula IF, L³ is —S(O)₂—, L⁴ is —C(O)—, and Y is substituted or unsubstituted furanyl.

In another embodiment of the compounds of formula IF, L³ is —C(R⁴)₂—, L⁴ is —S(O)₂—, and Y is substituted or unsubstituted furanyl.

In another embodiment of the compounds of formula IF, L³ is —CH₂—, L⁴ is —S(O)₂—, and Y is substituted or unsubstituted furanyl.

In another embodiment of the compounds of formula IF, L³ is —CH(CH₃)—, L⁴ is —S(O)₂—, and Y is substituted or unsubstituted furanyl.

In another embodiment of the compounds of formula IF, L³ is —C(O)—, L⁴ is —S(O)₂—, and Y is substituted or unsubstituted furanyl.

In another embodiment of the compounds of formula IF, L³ is —S(O)₂—, L⁴ is —S(O)₂—, and Y is substituted or unsubstituted furanyl.

In another embodiment of the compounds of formula IF, L³ is —C(R⁴)₂—, L⁴ is a covalent bond, and Y is substituted or unsubstituted pyrrolyl.

In another embodiment of the compounds of formula IF, L³ is —CH₂—, L⁴ is a covalent bond, and Y is substituted or unsubstituted pyrrolyl.

In another embodiment of the compounds of formula IF, L³ is —CH(CH₃)—, L⁴ is a covalent bond, and Y is substituted or unsubstituted pyrrolyl.

In another embodiment of the compounds of formula IF, L³ is —C(O)—, L⁴ is a covalent bond, and Y is substituted or unsubstituted pyrrolyl.

In another embodiment of the compounds of formula IF, L³ is —S(O)₂—, L⁴ is a covalent bond, and Y is substituted or unsubstituted pyrrolyl.

In another embodiment of the compounds of formula IF, L³ is —C(R⁴)₂—, L⁴ is —C(R⁴)₂—, and Y is substituted or unsubstituted pyrrolyl.

In another embodiment of the compounds of formula IF, L³ is —CH₂—, L⁴ is —C(R⁴)₂—, and Y is substituted or unsubstituted pyrrolyl.

In another embodiment of the compounds of formula IF, L³ is —CH(CH₃)—, L⁴ is —C(R⁴)₂—, and Y is substituted or unsubstituted pyrrolyl.

In another embodiment of the compounds of formula IF, L³ is —C(O)—, L⁴ is —C(R⁴)₂—, and Y is substituted or unsubstituted pyrrolyl.

In another embodiment of the compounds of formula IF, L³ is —S(O)₂—, L⁴ is —C(R⁴)₂—, and Y is substituted or unsubstituted pyrrolyl.

In another embodiment of the compounds of formula IF, L³ is —C(R⁴)₂—, L⁴ is —CH₂—, and Y is substituted or unsubstituted pyrrolyl.

In another embodiment of the compounds of formula IF, L³ is —CH₂—, L⁴ is —CH₂—, and Y is substituted or unsubstituted pyrrolyl.

In another embodiment of the compounds of formula IF, L³ is —CH(CH₃)—, L⁴ is —CH₂—, and Y is substituted or unsubstituted pyrrolyl.

In another embodiment of the compounds of formula IF, L³ is —C(O)—, L⁴ is —CH₂—, and Y is substituted or unsubstituted pyrrolyl.

In another embodiment of the compounds of formula IF, L³ is —S(O)₂—, L⁴ is —CH₂—, and Y is substituted or unsubstituted pyrrolyl.

In another embodiment of the compounds of formula IF, L³ is —C(R⁴)₂—, L⁴ is —CH(CH₃)—, and Y is substituted or unsubstituted pyrrolyl.

In another embodiment of the compounds of formula IF, L³ is —CH₂—, L⁴ is —CH(CH₃)—, and Y is substituted or unsubstituted pyrrolyl.

In another embodiment of the compounds of formula IF, L³ is —CH(CH₃)—, L⁴ is —CH(CH₃)—, and Y is substituted or unsubstituted pyrrolyl.

In another embodiment of the compounds of formula IF, L³ is —C(O)—, L⁴ is —CH(CH₃)—, and Y is substituted or unsubstituted pyrrolyl.

In another embodiment of the compounds of formula IF, L³ is —S(O)₂—, L⁴ is —CH(CH₃)—, and Y is substituted or unsubstituted pyrrolyl.

In another embodiment of the compounds of formula IF, L³ is —C(R⁴)₂—, L⁴ is —C(O)—, and Y is substituted or unsubstituted pyrrolyl.

In another embodiment of the compounds of formula IF, L³ is —CH₂—, L⁴ is —C(O)—, and Y is substituted or unsubstituted pyrrolyl.

In another embodiment of the compounds of formula IF, L³ is —CH(CH₃)—, L⁴ is —C(O)—, and Y is substituted or unsubstituted pyrrolyl.

In another embodiment of the compounds of formula IF, L³ is —C(O)—, L⁴ is —C(O)—, and Y is substituted or unsubstituted pyrrolyl.

In another embodiment of the compounds of formula IF, L³ is —S(O)₂—, L⁴ is —C(O)—, and Y is substituted or unsubstituted pyrrolyl.

In another embodiment of the compounds of formula IF, L³ is —C(R⁴)₂—, L⁴ is —S(O)₂—, and Y is substituted or unsubstituted pyrrolyl.

In another embodiment of the compounds of formula IF, L³ is —CH₂—, L⁴ is —S(O)₂—, and Y is substituted or unsubstituted pyrrolyl.

In another embodiment of the compounds of formula IF, L³ is —CH(CH₃)—, L⁴ is —S(O)₂—, and Y is substituted or unsubstituted pyrrolyl.

In another embodiment of the compounds of formula IF, L³ is —C(O)—, L⁴ is —S(O)₂—, and Y is substituted or unsubstituted pyrrolyl.

In another embodiment of the compounds of formula IF, L³ is —S(O)₂—, L⁴ is —S(O)₂—, and Y is substituted or unsubstituted pyrrolyl.

In another embodiment, the compounds of the present invention, or pharmaceutically acceptable salts, solvates, prodrugs, tautomers, or esters thereof, have the structure of formula (IG):

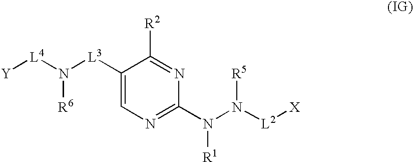

wherein R¹, R², R⁵, R⁶, L², L³, L⁴, X and Y are as defined herein.

In another embodiment of the compounds of formula IG, L² is —CH₂—; L³ is —C(O)— or —CH₂—; L⁴ is —S(O)₂— or —CH₂—; X is substituted or unsubstituted aryl; Y is selected from the group consisting of substituted or unsubstituted aryl, and substituted or unsubstituted heteroaryl; R¹, R⁵, and R⁶ are each H; R² is H, alkyl, halo, and haloalkyl.

In another embodiment of the compounds of formula IG, L² is —CH₂—; L³ is —C(O)— or —CH₂—; L⁴ is —S(O)₂— or —CH₂—; X is substituted or unsubstituted phenyl; Y is selected from the group consisting of substituted or unsubstituted phenyl, and substituted or unsubstituted benzimidazolyl; R¹, R⁵, and R⁶ are each H; R² is —CF₃.

In another embodiment of the compounds of formula IG, L² is a covalent bond.

In another embodiment of the compounds of formula IG, L² is —C(R⁴)₂—.

In another embodiment of the compounds of formula IG, L² is —CH₂—.

In another embodiment of the compounds of formula IG, L² is —CH(CH₃)—.

In another embodiment of the compounds of formula IG, L² is —C(O)—.

In another embodiment of the compounds of formula IG, L² is —S(O)—.

In another embodiment of the compounds of formula IG, L² is —S(O)₂—.

In another embodiment of the compounds of formula IG, R² is H.

In another embodiment of the compounds of formula IG, R² is haloalkyl.

In another embodiment of the compounds of formula IG, R² is —CF₃—.

In another embodiment of the compounds of formula IG, R¹ and R² are independently H or alkyl.

In another embodiment of the compounds of formula IG, R¹ and R⁵ are independently H or methyl.

In another embodiment of the compounds of formula IG, R¹ and R⁵ are independently H or substituted or unsubstituted arylalkyl.

In another embodiment of the compounds of formula IG, R¹ and R⁵ are both H.

In another embodiment of the compounds of formula IG, R¹ and R⁵, together with the nitrogen atoms to which they are shown attached, form a 5-membered heterocyclyl ring.

In another embodiment of the compounds of formula IG, R¹ and R⁵, together with the nitrogen atoms to which they are shown attached, form a 6-membered heterocyclyl ring.

In another embodiment of the compounds of formula IG, L² is a covalent bond, and R¹ and R⁵ are independently H or alkyl.

In another embodiment of the compounds of formula IG, L² is —C(R⁴)₂—, and R¹ and R⁵ are independently H or alkyl.

In another embodiment of the compounds of formula IG, L² is —CH₂—, and R¹ and R⁵ are independently H or alkyl.

In another embodiment of the compounds of formula IG, L² is —CH(CH₃)—, and R¹ and R⁵ are independently H or alkyl.

In another embodiment of the compounds of formula IG, L² is —C(O)—, and R¹ and R⁵ are independently H or alkyl.

In another embodiment of the compounds of formula IG, L² is a covalent bond, and R¹ and R⁵ are independently H or methyl.

In another embodiment of the compounds of formula IG, L² is —C(R⁴)₂—, and R¹ and R⁵ are independently H or methyl.

In another embodiment of the compounds of formula IG, L² is —CH₂—, and R¹ and R⁵ are independently H or methyl.

In another embodiment of the compounds of formula IG, L² is —CH(CH₃)—, and R¹ and R⁵ are independently H or methyl.

In another embodiment of the compounds of formula IG, L² is —C(O)—, and R¹ and R⁵ are independently H or methyl.

In another embodiment of the compounds of formula IG, L² is a covalent bond, and R¹ and R⁵ are independently H or substituted or unsubstituted arylalkyl.

In another embodiment of the compounds of formula IG, L² is —C(R⁴)₂—, and R¹ and R⁵ are independently H or substituted or unsubstituted arylalkyl.

In another embodiment of the compounds of formula IG, L² is —CH₂—, and R¹ and R⁵ are independently H or substituted or unsubstituted arylalkyl.

In another embodiment of the compounds of formula IG, L² is —CH(CH₃)—, and R¹ and R⁵ are independently H or substituted or unsubstituted arylalkyl.

In another embodiment of the compounds of formula IG, L² is —C(O)—, and R¹ and R⁵ are independently H or substituted or unsubstituted arylalkyl.

In another embodiment of the compounds of formula IG, L² is a covalent bond, and R¹ and R⁵ are both H.

In another embodiment of the compounds of formula IG, L² is —C(R⁴)₂—, and R¹ and R⁵ are both H.

In another embodiment of the compounds of formula IG, L² is —CH₂—, and R¹ and R⁵ are both H.

In another embodiment of the compounds of formula IG, L² is —CH(CH₃)—, and R¹ and R⁵ are both H.

In another embodiment of the compounds of formula IG, L² is —C(O)—, and R¹ and R⁵ are both H.

In another embodiment of the compounds of formula IG, L² is a covalent bond, and R¹ and R⁵, together with the nitrogen atoms to which they are shown attached, form a 5-membered heterocyclyl ring.

In another embodiment of the compounds of formula IG, L² is —C(R⁴)₂—, and R¹ and R⁵, together with the nitrogen atoms to which they are shown attached, form a 5-membered heterocyclyl ring.

In another embodiment of the compounds of formula IG, L² is —CH₂—, and R¹ and R⁵, together with the nitrogen atoms to which they are shown attached, form a 5-membered heterocyclyl ring.

In another embodiment of the compounds of formula IG, L² is —CH(CH₃)—, and R¹ and R⁵, together with the nitrogen atoms to which they are shown attached, form a 5-membered heterocyclyl ring.

In another embodiment of the compounds of formula IG, L² is —C(O)—, and R¹ and R⁵, together with the nitrogen atoms to which they are shown attached, form a 5-membered heterocyclyl ring.

In another embodiment of the compounds of formula IG, L² is a covalent bond, and R¹ and R⁵, together with the nitrogen atoms to which they are shown attached, form a 6-membered heterocyclyl ring.

In another embodiment of the compounds of formula IG, L² is —C(R⁴)₂—, and R¹ and R⁵, together with the nitrogen atoms to which they are shown attached, form a 6-membered heterocyclyl ring.

In another embodiment of the compounds of formula IG, L² is —CH₂—, and R¹ and R⁵, together with the nitrogen atoms to which they are shown attached, form a 6-membered heterocyclyl ring.

In another embodiment of the compounds of formula IG, L² is —CH(CH₃)—, and R¹ and R⁵, together with the nitrogen atoms to which they are shown attached, form a 6-membered heterocyclyl ring.

In another embodiment of the compounds of formula IG, L² is —C(O)—, and R¹ and R⁵, together with the nitrogen atoms to which they are shown attached, form a 6-membered heterocyclyl ring.

In another embodiment of the compounds of formula IG, L² is a covalent bond, R¹ and R⁵ are independently H or alkyl, and X is substituted or unsubstituted aryl.

In another embodiment of the compounds of formula IG, L² is —C(R⁴)₂—, R¹ and R⁵ are independently H or alkyl, and X is substituted or unsubstituted aryl.

In another embodiment of the compounds of formula IG, L² is —CH₂—, R¹ and R⁵ are independently H or alkyl, and X is substituted or unsubstituted aryl.

In another embodiment of the compounds of formula IG, L² is —CH(CH₃)—, R¹ and R⁵ are independently H or alkyl, and X is substituted or unsubstituted aryl.

In another embodiment of the compounds of formula IG, L² is —C(O)—, R¹ and R⁵ are independently H or alkyl, and X is substituted or unsubstituted aryl.

In another embodiment of the compounds of formula IG, L² is a covalent bond, R¹ and R⁵ are independently H or methyl, and X is substituted or unsubstituted aryl.

In another embodiment of the compounds of formula IG, L² is —C(R⁴)₂—, R¹ and R⁵ are independently H or methyl, and X is substituted or unsubstituted aryl.

In another embodiment of the compounds of formula IG, L² is —CH₂—, R¹ and R⁵ are independently H or methyl, and X is substituted or unsubstituted aryl.

In another embodiment of the compounds of formula IG, L² is —CH(CH₃)—, R¹ and R⁵ are independently H or methyl, and X is substituted or unsubstituted aryl.

In another embodiment of the compounds of formula IG, L² is —C(O)—, R¹ and R⁵ are independently H or methyl, and X is substituted or unsubstituted aryl.

In another embodiment of the compounds of formula IG, L² is a covalent bond, R¹ and R⁵ are independently H or substituted or unsubstituted arylalkyl, and X is substituted or unsubstituted aryl.

In another embodiment of the compounds of formula IG, L² is —C(R⁴)₂—, R¹ and R⁵ are independently H or substituted or unsubstituted arylalkyl, and X is substituted or unsubstituted aryl.

In another embodiment of the compounds of formula IG, L² is —CH₂—, R¹ and R⁵ are independently H or substituted or unsubstituted arylalkyl, and X is substituted or unsubstituted aryl.

In another embodiment of the compounds of formula IG, L² is —CH(CH₃)—, R¹ and R⁵ are independently H or substituted or unsubstituted arylalkyl, and X is substituted or unsubstituted aryl.

In another embodiment of the compounds of formula IG, L² is —C(O)—, R¹ and R⁵ are independently H or substituted or unsubstituted arylalkyl, and X is substituted or unsubstituted aryl.

In another embodiment of the compounds of formula IG, L² is a covalent bond, R¹ and R⁵ are both H, and X is substituted or unsubstituted aryl.

In another embodiment of the compounds of formula IG, L² is —C(R⁴)₂—, R¹ and R⁵ are both H, and X is substituted or unsubstituted aryl.

In another embodiment of the compounds of formula IG, L² is —CH₂—, R¹ and R⁵ are both H, and X is substituted or unsubstituted aryl.

In another embodiment of the compounds of formula IG, L² is —CH(CH₃)—, R¹ and R⁵ are both H, and X is substituted or unsubstituted aryl.

In another embodiment of the compounds of formula IG, L² is —C(O)—, R¹ and R⁵ are both H, and X is substituted or unsubstituted aryl.

In another embodiment of the compounds of formula IG, L² is a covalent bond, R¹ and R⁵, together with the nitrogen atoms to which they are shown attached, form a 5-membered heterocyclyl ring, and X is substituted or unsubstituted aryl.

In another embodiment of the compounds of formula IG, L² is —C(R⁴)₂—, R¹ and R⁵, together with the nitrogen atoms to which they are shown attached, form a 5-membered heterocyclyl ring, and X is substituted or unsubstituted aryl.

In another embodiment of the compounds of formula IG, L² is —CH₂—, R¹ and R⁵, together with the nitrogen atoms to which they are shown attached, form a 5-membered heterocyclyl ring, and X is substituted or unsubstituted aryl.

In another embodiment of the compounds of formula IG, L² is —CH(CH₃)—, R¹ and R⁵, together with the nitrogen atoms to which they are shown attached, form a 5-membered heterocyclyl ring, and X is substituted or unsubstituted aryl.

In another embodiment of the compounds of formula IG, L² is —C(O)—, R¹ and R⁵, together with the nitrogen atoms to which they are shown attached, form a 5-membered heterocyclyl ring, and X is substituted or unsubstituted aryl.

In another embodiment of the compounds of formula IG, L² is a covalent bond, R¹ and R⁵, together with the nitrogen atoms to which they are shown attached, form a 6-membered heterocyclyl ring, and X is substituted or unsubstituted aryl.

In another embodiment of the compounds of formula IG, L² is —C(R⁴)₂—, R¹ and R⁵, together with the nitrogen atoms to which they are shown attached, form a 6-membered heterocyclyl ring, and X is substituted or unsubstituted aryl.

In another embodiment of the compounds of formula IG, L² is —CH₂—, R¹ and R⁵, together with the nitrogen atoms to which they are shown attached, form a 6-membered heterocyclyl ring, and X is substituted or unsubstituted aryl.

In another embodiment of the compounds of formula IG, L² is —CH(CH₃)—, R¹ and R⁵, together with the nitrogen atoms to which they are shown attached, form a 6-membered heterocyclyl ring, and X is substituted or unsubstituted aryl.

In another embodiment of the compounds of formula IG, L² is —C(O)—, R¹ and R⁵, together with the nitrogen atoms to which they are shown attached, form a 6-membered heterocyclyl ring, and X is substituted or unsubstituted aryl.

In another embodiment of the compounds of formula IG, L² is a covalent bond, R¹ and R⁵ are independently H or alkyl, and X is substituted or unsubstituted phenyl.

In another embodiment of the compounds of formula IG, L² is —C(R⁴)₂—, R¹ and R⁵ are independently H or alkyl, and X is substituted or unsubstituted phenyl.

In another embodiment of the compounds of formula IG, L² is —CH₂—, R¹ and R⁵ are independently H or alkyl, and X is substituted or unsubstituted phenyl.

In another embodiment of the compounds of formula IG, L² is —CH(CH₃)—, R¹ and R⁵ are independently H or alkyl, and X is substituted or unsubstituted phenyl.

In another embodiment of the compounds of formula IG, L² is —C(O)—, R¹ and R⁵ are independently H or alkyl, and X is substituted or unsubstituted phenyl.

In another embodiment of the compounds of formula IG, L² is a covalent bond, R¹ and R⁵ are independently H or methyl, and X is substituted or unsubstituted phenyl.

In another embodiment of the compounds of formula IG, L² is —C(R⁴)₂—, R¹ and R⁵ are independently H or methyl, and X is substituted or unsubstituted phenyl.

In another embodiment of the compounds of formula IG, L² is —CH₂—, R¹ and R⁵ are independently H or methyl, and X is substituted or unsubstituted phenyl.

In another embodiment of the compounds of formula IG, L² is —CH(CH₃)—, R¹ and R⁵ are independently H or methyl, and X is substituted or unsubstituted phenyl.

In another embodiment of the compounds of formula IG, L² is —C(O)—, R¹ and R⁵ are independently H or methyl, and X is substituted or unsubstituted phenyl.

In another embodiment of the compounds of formula IG, L² is a covalent bond, R¹ and R⁵ are independently H or substituted or unsubstituted arylalkyl, and X is substituted or unsubstituted phenyl.

In another embodiment of the compounds of formula IG, L² is —C(R⁴)₂—, R¹ and R⁵ are independently H or substituted or unsubstituted arylalkyl, and X is substituted or unsubstituted phenyl.

In another embodiment of the compounds of formula IG, L² is —CH₂—, R¹ and R⁵ are independently H or substituted or unsubstituted arylalkyl, and X is substituted or unsubstituted phenyl.

In another embodiment of the compounds of formula IG, L² is —CH(CH₃)—, R¹ and R⁵ are independently H or substituted or unsubstituted arylalkyl, and X is substituted or unsubstituted phenyl.

In another embodiment of the compounds of formula IG, L² is —C(O)—, R¹ and R⁵ are independently H or substituted or unsubstituted arylalkyl, and X is substituted or unsubstituted phenyl.

In another embodiment of the compounds of formula IG, L² is a covalent bond, R¹ and R⁵ are both H, and X is substituted or unsubstituted phenyl.

In another embodiment of the compounds of formula IG, L² is —C(R⁴)₂—, R¹ and R⁵ are both H, and X is substituted or unsubstituted phenyl.

In another embodiment of the compounds of formula IG, L² is —CH₂—, R¹ and R⁵ are both H, and X is substituted or unsubstituted phenyl.

In another embodiment of the compounds of formula IG, L² is —CH(CH₃)—, R¹ and R⁵ are both H, and X is substituted or unsubstituted phenyl.

In another embodiment of the compounds of formula IG, L² is —C(O)—, R¹ and R⁵ are both H, and X is substituted or unsubstituted phenyl.

In another embodiment of the compounds of formula IG, L² is a covalent bond, R¹ and R⁵, together with the nitrogen atoms to which they are shown attached, form a 5-membered heterocyclyl ring, and X is substituted or unsubstituted phenyl.

In another embodiment of the compounds of formula IG, L² is —C(R⁴)₂—, R¹ and R⁵, together with the nitrogen atoms to which they are shown attached, form a 5-membered heterocyclyl ring, and X is substituted or unsubstituted phenyl.

In another embodiment of the compounds of formula IG, L² is —CH₂—, R¹ and R⁵, together with the nitrogen atoms to which they are shown attached, form a 5-membered heterocyclyl ring, and X is substituted or unsubstituted phenyl.

In another embodiment of the compounds of formula IG, L² is —CH(CH₃)—, R¹ and R⁵, together with the nitrogen atoms to which they are shown attached, form a 5-membered heterocyclyl ring, and X is substituted or unsubstituted phenyl.

In another embodiment of the compounds of formula IG, L² is —C(O)—, R¹ and R⁵, together with the nitrogen atoms to which they are shown attached, form a 5-membered heterocyclyl ring, and X is substituted or unsubstituted phenyl.

In another embodiment of the compounds of formula IG, L² is a covalent bond, R¹ and R⁵, together with the nitrogen atoms to which they are shown attached, form a 6-membered heterocyclyl ring, and X is substituted or unsubstituted phenyl.

In another embodiment of the compounds of formula IG, L² is —C(R⁴)₂—, R¹ and R⁵, together with the nitrogen atoms to which they are shown attached, form a 6-membered heterocyclyl ring, and X is substituted or unsubstituted phenyl.

In another embodiment of the compounds of formula IG, L² is —CH₂—, R¹ and R⁵, together with the nitrogen atoms to which they are shown attached, form a 6-membered heterocyclyl ring, and X is substituted or unsubstituted phenyl.

In another embodiment of the compounds of formula IG, L² is —CH(CH₃)—, R¹ and R⁵, together with the nitrogen atoms to which they are shown attached, form a 6-membered heterocyclyl ring, and X is substituted or unsubstituted phenyl.

In another embodiment of the compounds of formula IG, L² is —C(O)—, R¹ and R⁵, together with the nitrogen atoms to which they are shown attached, form a 6-membered heterocyclyl ring, and X is substituted or unsubstituted phenyl.

In another embodiment of the compounds of formula IG, L² is a covalent bond, R¹ and R⁵ are independently H or alkyl, and X is substituted or unsubstituted heteroaryl.

In another embodiment of the compounds of formula IG, L² is —C(R⁴)₂—, R¹ and R⁵ are independently H or alkyl, and X is substituted or unsubstituted heteroaryl.

In another embodiment of the compounds of formula IG, L² is —CH₂—, R¹ and R⁵ are independently H or alkyl, and X is substituted or unsubstituted heteroaryl.

In another embodiment of the compounds of formula IG, L² is —CH(CH₃)—, R¹ and R⁵ are independently H or alkyl, and X is substituted or unsubstituted heteroaryl.

In another embodiment of the compounds of formula IG, L² is —C(O)—, R¹ and R⁵ are independently H or alkyl, and X is substituted or unsubstituted heteroaryl.

In another embodiment of the compounds of formula IG, L² is a covalent bond, R¹ and R⁵ are independently H or methyl, and X is substituted or unsubstituted heteroaryl.

In another embodiment of the compounds of formula IG, L² is —C(R⁴)₂—, R¹ and R⁵ are independently H or methyl, and X is substituted or unsubstituted heteroaryl.

In another embodiment of the compounds of formula IG, L² is —CH₂—, R¹ and R⁵ are independently H or methyl, and X is substituted or unsubstituted heteroaryl.

In another embodiment of the compounds of formula IG, L² is —CH(CH₃)—, R¹ and R⁵ are independently H or methyl, and X is substituted or unsubstituted heteroaryl.

In another embodiment of the compounds of formula IG, L² is —C(O)—, R¹ and R⁵ are independently H or methyl, and X is substituted or unsubstituted heteroaryl.

In another embodiment of the compounds of formula IG, L² is a covalent bond, R¹ and R⁵ are independently H or substituted or unsubstituted arylalkyl, and X is substituted or unsubstituted heteroaryl.

In another embodiment of the compounds of formula IG, L² is —C(R⁴)₂—, R¹ and R⁵ are independently H or substituted or unsubstituted arylalkyl, and X is substituted or unsubstituted heteroaryl.

In another embodiment of the compounds of formula IG, L² is —CH₂—, R¹ and R⁵ are independently H or substituted or unsubstituted arylalkyl, and X is substituted or unsubstituted heteroaryl.

In another embodiment of the compounds of formula IG, L² is —CH(CH₃)—, R¹ and R⁵ are independently H or substituted or unsubstituted arylalkyl, and X is substituted or unsubstituted heteroaryl.

In another embodiment of the compounds of formula IG, L² is —C(O)—, R¹ and R⁵ are independently H or substituted or unsubstituted arylalkyl, and X is substituted or unsubstituted heteroaryl.

In another embodiment of the compounds of formula IG, L² is a covalent bond, R¹ and R⁵ are both H, and X is substituted or unsubstituted heteroaryl.

In another embodiment of the compounds of formula IG, L² is —C(R⁴)₂—, R¹ and R⁵ are both H, and X is substituted or unsubstituted heteroaryl.

In another embodiment of the compounds of formula IG, L² is —CH₂—, R¹ and R⁵ are both H, and X is substituted or unsubstituted heteroaryl.

In another embodiment of the compounds of formula IG, L² is —CH(CH₃)—, R¹ and R⁵ are both H, and X is substituted or unsubstituted heteroaryl.

In another embodiment of the compounds of formula IG, L² is —C(O)—, R¹ and R⁵ are both H, and X is substituted or unsubstituted heteroaryl.

In another embodiment of the compounds of formula IG, L² is a covalent bond, R¹ and R⁵, together with the nitrogen atoms to which they are shown attached, form a 5-membered heterocyclyl ring, and X is substituted or unsubstituted heteroaryl.

In another embodiment of the compounds of formula IG, L² is —C(R⁴)₂—, R¹ and R⁵, together with the nitrogen atoms to which they are shown attached, form a 5-membered heterocyclyl ring, and X is substituted or unsubstituted heteroaryl.

In another embodiment of the compounds of formula IG, L² is —CH₂—, R¹ and R⁵, together with the nitrogen atoms to which they are shown attached, form a 5-membered heterocyclyl ring, and X is substituted or unsubstituted heteroaryl.

In another embodiment of the compounds of formula IG, L² is —CH(CH₃)—, R¹ and R⁵, together with the nitrogen atoms to which they are shown attached, form a 5-membered heterocyclyl ring, and X is substituted or unsubstituted heteroaryl.

In another embodiment of the compounds of formula IG, L² is —C(O)—, R¹ and R⁵, together with the nitrogen atoms to which they are shown attached, form a 5-membered heterocyclyl ring, and X is substituted or unsubstituted heteroaryl.

In another embodiment of the compounds of formula IG, L² is a covalent bond, R¹ and R⁵, together with the nitrogen atoms to which they are shown attached, form a 6-membered heterocyclyl ring, and X is substituted or unsubstituted heteroaryl.

In another embodiment of the compounds of formula IG, L² is —C(R⁴)₂—, R¹ and R⁵, together with the nitrogen atoms to which they are shown attached, form a 6-membered heterocyclyl ring, and X is substituted or unsubstituted heteroaryl.

In another embodiment of the compounds of formula IG, L² is —CH₂—, R¹ and R⁵, together with the nitrogen atoms to which they are shown attached, form a 6-membered heterocyclyl ring, and X is substituted or unsubstituted heteroaryl.

In another embodiment of the compounds of formula IG, L² is —CH(CH₃)—, R¹ and R⁵, together with the nitrogen atoms to which they are shown attached, form a 6-membered heterocyclyl ring, and X is substituted or unsubstituted heteroaryl.

In another embodiment of the compounds of formula IG, L² is —C(O)—, R¹ and R⁵, together with the nitrogen atoms to which they are shown attached, form a 6-membered heterocyclyl ring, and X is substituted or unsubstituted heteroaryl.

In another embodiment of the compounds of formula IG, L² is a covalent bond, R¹ and R⁵ are independently H or alkyl, and X is substituted or unsubstituted pyridyl.

In another embodiment of the compounds of formula IG, L² is —C(R⁴)₂—, R¹ and R⁵ are independently H or alkyl, and X is substituted or unsubstituted pyridyl.

In another embodiment of the compounds of formula IG, L² is —CH₂—, R¹ and R⁵ are independently H or alkyl, and X is substituted or unsubstituted pyridyl.

In another embodiment of the compounds of formula IG, L² is —CH(CH₃)—, R¹ and R⁵ are independently H or alkyl, and X is substituted or unsubstituted pyridyl.

In another embodiment of the compounds of formula IG, L² is —C(O)—, R¹ and R⁵ are independently H or alkyl, and X is substituted or unsubstituted pyridyl.

In another embodiment of the compounds of formula IG, L² is a covalent bond, R¹ and R⁵ are independently H or methyl, and X is substituted or unsubstituted pyridyl.

In another embodiment of the compounds of formula IG, L² is —C(R⁴)₂—, R¹ and R⁵ are independently H or methyl, and X is substituted or unsubstituted pyridyl.

In another embodiment of the compounds of formula IG, L² is —CH₂—, R¹ and R⁵ are independently H or methyl, and X is substituted or unsubstituted pyridyl.

In another embodiment of the compounds of formula IG, L² is —CH(CH₃)—, R¹ and R⁵ are independently H or methyl, and X is substituted or unsubstituted pyridyl.

In another embodiment of the compounds of formula IG, L² is —C(O)—, R¹ and R⁵ are independently H or methyl, and X is substituted or unsubstituted pyridyl.

In another embodiment of the compounds of formula IG, L² is a covalent bond, R¹ and R⁵ are independently H or substituted or unsubstituted arylalkyl, and X is substituted or unsubstituted pyridyl.

In another embodiment of the compounds of formula IG, L² is —C(R⁴)₂—, R¹ and R⁵ are independently H or substituted or unsubstituted arylalkyl, and X is substituted or unsubstituted pyridyl.

In another embodiment of the compounds of formula IG, L² is —CH₂—, R¹ and R⁵ are independently H or substituted or unsubstituted arylalkyl, and X is substituted or unsubstituted pyridyl.

In another embodiment of the compounds of formula IG, L² is —CH(CH₃)—, R¹ and R⁵ are independently H or substituted or unsubstituted arylalkyl, and X is substituted or unsubstituted pyridyl.

In another embodiment of the compounds of formula IG, L² is —C(O)—, R¹ and R⁵ are independently H or substituted or unsubstituted arylalkyl, and X is substituted or unsubstituted pyridyl.

In another embodiment of the compounds of formula IG, L² is a covalent bond, R¹ and R⁵ are both H, and X is substituted or unsubstituted pyridyl.

In another embodiment of the compounds of formula IG, L² is —C(R⁴)₂—, R¹ and R⁵ are both H, and X is substituted or unsubstituted pyridyl.

In another embodiment of the compounds of formula IG, L² is —CH₂—, R¹ and R⁵ are both H, and X is substituted or unsubstituted pyridyl.

In another embodiment of the compounds of formula IG, L² is —CH(CH₃)—, R¹ and R⁵ are both H, and X is substituted or unsubstituted pyridyl.

In another embodiment of the compounds of formula IG, L² is —C(O)—, R¹ and R⁵ are both H, and X is substituted or unsubstituted pyridyl.

In another embodiment of the compounds of formula IG, L² is a covalent bond, R¹ and R⁵, together with the nitrogen atoms to which they are shown attached, form a 5-membered heterocyclyl ring, and X is substituted or unsubstituted pyridyl.

In another embodiment of the compounds of formula IG, L² is —C(R⁴)₂—, R¹ and R⁵, together with the nitrogen atoms to which they are shown attached, form a 5-membered heterocyclyl ring, and X is substituted or unsubstituted pyridyl.

In another embodiment of the compounds of formula IG, L² is —CH₂—, R¹ and R⁵, together with the nitrogen atoms to which they are shown attached, form a 5-membered heterocyclyl ring, and X is substituted or unsubstituted pyridyl.

In another embodiment of the compounds of formula IG, L² is —CH(CH₃)—, R¹ and R⁵, together with the nitrogen atoms to which they are shown attached, form a 5-membered heterocyclyl ring, and X is substituted or unsubstituted pyridyl.

In another embodiment of the compounds of formula IG, L² is —C(O)—, R¹ and R⁵, together with the nitrogen atoms to which they are shown attached, form a 5-membered heterocyclyl ring, and X is substituted or unsubstituted pyridyl.

In another embodiment of the compounds of formula IG, L² is a covalent bond, R¹ and R⁵, together with the nitrogen atoms to which they are shown attached, form a 6-membered heterocyclyl ring, and X is substituted or unsubstituted pyridyl.

In another embodiment of the compounds of formula IG, L² is —C(R⁴)₂—, R¹ and R⁵, together with the nitrogen atoms to which they are shown attached, form a 6-membered heterocyclyl ring, and X is substituted or unsubstituted pyridyl.

In another embodiment of the compounds of formula IG, L² is —CH₂—, R¹ and R⁵, together with the nitrogen atoms to which they are shown attached, form a 6-membered heterocyclyl ring, and X is substituted or unsubstituted pyridyl.

In another embodiment of the compounds of formula IG, L² is —CH(CH₃)—, R¹ and R⁵, together with the nitrogen atoms to which they are shown attached, form a 6-membered heterocyclyl ring, and X is substituted or unsubstituted pyridyl.

In another embodiment of the compounds of formula IG, L² is —C(O)—, R¹ and R⁵, together with the nitrogen atoms to which they are shown attached, form a 6-membered heterocyclyl ring, and X is substituted or unsubstituted pyridyl.

In another embodiment of the compounds of formula IG, L² is a covalent bond, R¹ and R⁵ are independently H or alkyl, and X is substituted or unsubstituted pyrimidyl.

In another embodiment of the compounds of formula IG, L² is —C(R⁴)₂—, R¹ and R⁵ are independently H or alkyl, and X is substituted or unsubstituted pyrimidyl.

In another embodiment of the compounds of formula IG, L² is —CH₂—, R¹ and R⁵ are independently H or alkyl, and X is substituted or unsubstituted pyrimidyl.

In another embodiment of the compounds of formula IG, L² is —CH(CH₃)—, R¹ and R⁵ are independently H or alkyl, and X is substituted or unsubstituted pyrimidyl.

In another embodiment of the compounds of formula IG, L² is —C(O)—, R¹ and R⁵ are independently H or alkyl, and X is substituted or unsubstituted pyrimidyl.

In another embodiment of the compounds of formula IG, L² is a covalent bond, R¹ and R⁵ are independently H or methyl, and X is substituted or unsubstituted pyrimidyl.

In another embodiment of the compounds of formula IG, L² is —C(R⁴)₂—, R¹ and R⁵ are independently H or methyl, and X is substituted or unsubstituted pyrimidyl.

In another embodiment of the compounds of formula IG, L² is —CH₂—, R¹ and R⁵ are independently H or methyl, and X is substituted or unsubstituted pyrimidyl.

In another embodiment of the compounds of formula IG, L² is —CH(CH₃)—, R¹ and R⁵ are independently H or methyl, and X is substituted or unsubstituted pyrimidyl.

In another embodiment of the compounds of formula IG, L² is —C(O)—, R¹ and R⁵ are independently H or methyl, and X is substituted or unsubstituted pyrimidyl.

In another embodiment of the compounds of formula IG, L² is a covalent bond, R¹ and R⁵ are independently H or substituted or unsubstituted arylalkyl, and X is substituted or unsubstituted pyrimidyl.

In another embodiment of the compounds of formula IG, L² is —C(R⁴)₂—, R¹ and R⁵ are independently H or substituted or unsubstituted arylalkyl, and X is substituted or unsubstituted pyrimidyl.

In another embodiment of the compounds of formula IG, L² is —CH₂—, R¹ and R⁵ are independently H or substituted or unsubstituted arylalkyl, and X is substituted or unsubstituted pyrimidyl.

In another embodiment of the compounds of formula IG, L² is —CH(CH₃)—, R¹ and R⁵ are independently H or substituted or unsubstituted arylalkyl, and X is substituted or unsubstituted pyrimidyl.

In another embodiment of the compounds of formula IG, L² is —C(O)—, R¹ and R⁵ are independently H or substituted or unsubstituted arylalkyl, and X is substituted or unsubstituted pyrimidyl.

In another embodiment of the compounds of formula IG, L² is a covalent bond, R¹ and R⁵ are both H, and X is substituted or unsubstituted pyrimidyl.

In another embodiment of the compounds of formula IG, L² is —C(R⁴)₂—, R¹ and R⁵ are both H, and X is substituted or unsubstituted pyrimidyl.

In another embodiment of the compounds of formula IG, L² is —CH₂—, R¹ and R⁵ are both H, and X is substituted or unsubstituted pyrimidyl.

In another embodiment of the compounds of formula IG, L² is —CH(CH₃)—, R¹ and R⁵ are both H, and X is substituted or unsubstituted pyrimidyl.

In another embodiment of the compounds of formula IG, L² is —C(O)—, R¹ and R⁵ are both H, and X is substituted or unsubstituted pyrimidyl.

In another embodiment of the compounds of formula IG, L² is a covalent bond, R¹ and R⁵, together with the nitrogen atoms to which they are shown attached, form a 5-membered heterocyclyl ring, and X is substituted or unsubstituted pyrimidyl.

In another embodiment of the compounds of formula IG, L² is —C(R⁴)₂—, R¹ and R⁵, together with the nitrogen atoms to which they are shown attached, form a 5-membered heterocyclyl ring, and X is substituted or unsubstituted pyrimidyl.

In another embodiment of the compounds of formula IG, L² is —CH₂—, R¹ and R⁵, together with the nitrogen atoms to which they are shown attached, form a 5-membered heterocyclyl ring, and X is substituted or unsubstituted pyrimidyl.

In another embodiment of the compounds of formula IG, L² is —CH(CH₃)—, R¹ and R⁵, together with the nitrogen atoms to which they are shown attached, form a 5-membered heterocyclyl ring, and X is substituted or unsubstituted pyrimidyl.

In another embodiment of the compounds of formula IG, L² is —C(O)—, R¹ and R⁵, together with the nitrogen atoms to which they are shown attached, form a 5-membered heterocyclyl ring, and X is substituted or unsubstituted pyrimidyl.

In another embodiment of the compounds of formula IG, L² is a covalent bond, R¹ and R⁵, together with the nitrogen atoms to which they are shown attached, form a 6-membered heterocyclyl ring, and X is substituted or unsubstituted pyrimidyl.

In another embodiment of the compounds of formula IG, L² is —C(R⁴)₂—, R¹ and R⁵, together with the nitrogen atoms to which they are shown attached, form a 6-membered heterocyclyl ring, and X is substituted or unsubstituted pyrimidyl.

In another embodiment of the compounds of formula IG, L² is —CH₂—, R¹ and R⁵, together with the nitrogen atoms to which they are shown attached, form a 6-membered heterocyclyl ring, and X is substituted or unsubstituted pyrimidyl.

In another embodiment of the compounds of formula IG, L² is —CH(CH₃)—, R¹ and R⁵, together with the nitrogen atoms to which they are shown attached, form a 6-membered heterocyclyl ring, and X is substituted or unsubstituted pyrimidyl.

In another embodiment of the compounds of formula IG, L² is —C(O)—, R¹ and R⁵, together with the nitrogen atoms to which they are shown attached, form a 6-membered heterocyclyl ring, and X is substituted or unsubstituted pyrimidyl.

In another embodiment of the compounds of formula IG, L² is a covalent bond, R¹ and R⁵ are independently H or alkyl, and X is substituted or unsubstituted indolyl.

In another embodiment of the compounds of formula IG, L² is —C(R⁴)₂—, R¹ and R⁵ are independently H or alkyl, and X is substituted or unsubstituted indolyl.

In another embodiment of the compounds of formula IG, L² is —CH₂—, R¹ and R⁵ are independently H or alkyl, and X is substituted or unsubstituted indolyl.

In another embodiment of the compounds of formula IG, L² is —CH(CH₃)—, R¹ and R⁵ are independently H or alkyl, and X is substituted or unsubstituted indolyl.

In another embodiment of the compounds of formula IG, L² is —C(O)—, R¹ and R⁵ are independently H or alkyl, and X is substituted or unsubstituted indolyl.

In another embodiment of the compounds of formula IG, L² is a covalent bond, R¹ and R⁵ are independently H or methyl, and X is substituted or unsubstituted indolyl.

In another embodiment of the compounds of formula IG, L² is —C(R⁴)₂—, R¹ and R⁵ are independently H or methyl, and X is substituted or unsubstituted indolyl.

In another embodiment of the compounds of formula IG, L² is —CH₂—, R¹ and R⁵ are independently H or methyl, and X is substituted or unsubstituted indolyl.

In another embodiment of the compounds of formula IG, L² is —CH(CH₃)—, R¹ and R⁵ are independently H or methyl, and X is substituted or unsubstituted indolyl.

In another embodiment of the compounds of formula IG, L² is —C(O)—, R¹ and R⁵ are independently H or methyl, and X is substituted or unsubstituted indolyl.

In another embodiment of the compounds of formula IG, L² is a covalent bond, R¹ and R⁵ are independently H or substituted or unsubstituted arylalkyl, and X is substituted or unsubstituted indolyl.

In another embodiment of the compounds of formula IG, L² is —C(R⁴)₂—, R¹ and R⁵ are independently H or substituted or unsubstituted arylalkyl, and X is substituted or unsubstituted indolyl.

In another embodiment of the compounds of formula IG, L² is —CH₂—, R¹ and R⁵ are independently H or substituted or unsubstituted arylalkyl, and X is substituted or unsubstituted indolyl.

In another embodiment of the compounds of formula IG, L² is —CH(CH₃)—, R¹ and R⁵ are independently H or substituted or unsubstituted arylalkyl, and X is substituted or unsubstituted indolyl.

In another embodiment of the compounds of formula IG, L² is —C(O)—, R¹ and R⁵ are independently H or substituted or unsubstituted arylalkyl, and X is substituted or unsubstituted indolyl.

In another embodiment of the compounds of formula IG, L² is a covalent bond, R¹ and R⁵ are both H, and X is substituted or unsubstituted indolyl.

In another embodiment of the compounds of formula IG, L² is —C(R⁴)₂—, R¹ and R⁵ are both H, and X is substituted or unsubstituted indolyl.

In another embodiment of the compounds of formula IG, L² is —CH₂—, R¹ and R⁵ are both H, and X is substituted or unsubstituted indolyl.

In another embodiment of the compounds of formula IG, L² is —CH(CH₃)—, R¹ and R⁵ are both H, and X is substituted or unsubstituted indolyl.

In another embodiment of the compounds of formula IG, L² is —C(O)—, R¹ and R⁵ are both H, and X is substituted or unsubstituted indolyl.

In another embodiment of the compounds of formula IG, L² is a covalent bond, R¹ and R⁵, together with the nitrogen atoms to which they are shown attached, form a 5-membered heterocyclyl ring, and X is substituted or unsubstituted indolyl.

In another embodiment of the compounds of formula IG, L² is —C(R⁴)₂—, R¹ and R⁵, together with the nitrogen atoms to which they are shown attached, form a 5-membered heterocyclyl ring, and X is substituted or unsubstituted indolyl.

In another embodiment of the compounds of formula IG, L² is —CH₂—, R¹ and R⁵, together with the nitrogen atoms to which they are shown attached, form a 5-membered heterocyclyl ring, and X is substituted or unsubstituted indolyl.

In another embodiment of the compounds of formula IG, L² is —CH(CH₃)—, R¹ and R⁵, together with the nitrogen atoms to which they are shown attached, form a 5-membered heterocyclyl ring, and X is substituted or unsubstituted indolyl.

In another embodiment of the compounds of formula IG, L² is —C(O)—, R¹ and R⁵, together with the nitrogen atoms to which they are shown attached, form a 5-membered heterocyclyl ring, and X is substituted or unsubstituted indolyl.

In another embodiment of the compounds of formula IG, L² is a covalent bond, R¹ and R⁵, together with the nitrogen atoms to which they are shown attached, form a 6-membered heterocyclyl ring, and X is substituted or unsubstituted indolyl.

In another embodiment of the compounds of formula IG, L² is —C(R⁴)₂—, R¹ and R⁵, together with the nitrogen atoms to which they are shown attached, form a 6-membered heterocyclyl ring, and X is substituted or unsubstituted indolyl.

In another embodiment of the compounds of formula IG, L² is —CH₂—, R¹ and R⁵, together with the nitrogen atoms to which they are shown attached, form a 6-membered heterocyclyl ring, and X is substituted or unsubstituted indolyl.

In another embodiment of the compounds of formula IG, L² is —CH(CH₃)—, R¹ and R⁵, together with the nitrogen atoms to which they are shown attached, form a 6-membered heterocyclyl ring, and X is substituted or unsubstituted indolyl.

In another embodiment of the compounds of formula IG, L² is —C(O)—, R¹ and R⁵, together with the nitrogen atoms to which they are shown attached, form a 6-membered heterocyclyl ring, and X is substituted or unsubstituted indolyl.

In another embodiment of the compounds of formula IG, L² is a covalent bond, R¹ and R⁵ are independently H or alkyl, and X is substituted or unsubstituted benzimidazolyl.

In another embodiment of the compounds of formula IG, L² is —C(R⁴)₂—, R¹ and R⁵ are independently H or alkyl, and X is substituted or unsubstituted benzimidazolyl.

In another embodiment of the compounds of formula IG, L² is —CH₂—, R¹ and R⁵ are independently H or alkyl, and X is substituted or unsubstituted benzimidazolyl.

In another embodiment of the compounds of formula IG, L² is —CH(CH₃)—, R¹ and R⁵ are independently H or alkyl, and X is substituted or unsubstituted benzimidazolyl.

In another embodiment of the compounds of formula IG, L² is —C(O)—, R¹ and R⁵ are independently H or alkyl, and X is substituted or unsubstituted benzimidazolyl.

In another embodiment of the compounds of formula IG, L² is a covalent bond, R¹ and R⁵ are independently H or methyl, and X is substituted or unsubstituted benzimidazolyl.

In another embodiment of the compounds of formula IG, L² is —C(R⁴)₂—, R¹ and R⁵ are independently H or methyl, and X is substituted or unsubstituted benzimidazolyl.

In another embodiment of the compounds of formula IG, L² is —CH₂—, R¹ and R⁵ are independently H or methyl, and X is substituted or unsubstituted benzimidazolyl.

In another embodiment of the compounds of formula IG, L² is —CH(CH₃)—, R¹ and R⁵ are independently H or methyl, and X is substituted or unsubstituted benzimidazolyl.

In another embodiment of the compounds of formula IG, L² is —C(O)—, R¹ and R⁵ are independently H or methyl, and X is substituted or unsubstituted benzimidazolyl.

In another embodiment of the compounds of formula IG, L² is a covalent bond, R¹ and R⁵ are independently H or substituted or unsubstituted arylalkyl, and X is substituted or unsubstituted benzimidazolyl.

In another embodiment of the compounds of formula IG, L² is —C(R⁴)₂—, R¹ and R⁵ are independently H or substituted or unsubstituted arylalkyl, and X is substituted or unsubstituted benzimidazolyl.

In another embodiment of the compounds of formula IG, L² is —CH₂—, R¹ and R⁵ are independently H or substituted or unsubstituted arylalkyl, and X is substituted or unsubstituted benzimidazolyl.

In another embodiment of the compounds of formula IG, L² is —CH(CH₃)—, R¹ and R⁵ are independently H or substituted or unsubstituted arylalkyl, and X is substituted or unsubstituted benzimidazolyl.

In another embodiment of the compounds of formula IG, L² is —C(O)—, R¹ and R⁵ are independently H or substituted or unsubstituted arylalkyl, and X is substituted or unsubstituted benzimidazolyl.

In another embodiment of the compounds of formula IG, L² is a covalent bond, R¹ and R⁵ are both H, and X is substituted or unsubstituted benzimidazolyl.

In another embodiment of the compounds of formula IG, L² is —C(R⁴)₂—, R¹ and R⁵ are both H, and X is substituted or unsubstituted benzimidazolyl.

In another embodiment of the compounds of formula IG, L² is —CH₂—, R¹ and R⁵ are both H, and X is substituted or unsubstituted benzimidazolyl.

In another embodiment of the compounds of formula IG, L² is —CH(CH₃)—, R¹ and R⁵ are both H, and X is substituted or unsubstituted benzimidazolyl.

In another embodiment of the compounds of formula IG, L² is —C(O)—, R¹ and R⁵ are both H, and X is substituted or unsubstituted benzimidazolyl.

In another embodiment of the compounds of formula IG, L² is a covalent bond, R¹ and R⁵, together with the nitrogen atoms to which they are shown attached, form a 5-membered heterocyclyl ring, and X is substituted or unsubstituted benzimidazolyl.

In another embodiment of the compounds of formula IG, L² is —C(R⁴)₂—, R¹ and R⁵, together with the nitrogen atoms to which they are shown attached, form a 5-membered heterocyclyl ring, and X is substituted or unsubstituted benzimidazolyl.

In another embodiment of the compounds of formula IG, L² is —CH₂—, R¹ and R⁵, together with the nitrogen atoms to which they are shown attached, form a 5-membered heterocyclyl ring, and X is substituted or unsubstituted benzimidazolyl.

In another embodiment of the compounds of formula IG, L² is —CH(CH₃)—, R¹ and R⁵, together with the nitrogen atoms to which they are shown attached, form a 5-membered heterocyclyl ring, and X is substituted or unsubstituted benzimidazolyl.

In another embodiment of the compounds of formula IG, L² is —C(O)—, R¹ and R⁵, together with the nitrogen atoms to which they are shown attached, form a 5-membered heterocyclyl ring, and X is substituted or unsubstituted benzimidazolyl.

In another embodiment of the compounds of formula IG, L² is a covalent bond, R¹ and R⁵, together with the nitrogen atoms to which they are shown attached, form a 6-membered heterocyclyl ring, and X is substituted or unsubstituted benzimidazolyl.

In another embodiment of the compounds of formula IG, L² is —C(R⁴)₂—, R¹ and R⁵, together with the nitrogen atoms to which they are shown attached, form a 6-membered heterocyclyl ring, and X is substituted or unsubstituted benzimidazolyl.

In another embodiment of the compounds of formula IG, L² is —CH₂—, R¹ and R⁵, together with the nitrogen atoms to which they are shown attached, form a 6-membered heterocyclyl ring, and X is substituted or unsubstituted benzimidazolyl.

In another embodiment of the compounds of formula IG, L² is —CH(CH₃)—, R¹ and R⁵, together with the nitrogen atoms to which they are shown attached, form a 6-membered heterocyclyl ring, and X is substituted or unsubstituted benzimidazolyl.

In another embodiment of the compounds of formula IG, L² is —C(O)—, R¹ and R⁵, together with the nitrogen atoms to which they are shown attached, form a 6-membered heterocyclyl ring, and X is substituted or unsubstituted benzimidazolyl.

In another embodiment of the compounds of formula IG, L² is a covalent bond, R¹ and R⁵ are independently H or alkyl, and X is substituted or unsubstituted purinyl.

In another embodiment of the compounds of formula IG, L² is —C(R⁴)₂—, R¹ and R⁵ are independently H or alkyl, and X is substituted or unsubstituted purinyl.

In another embodiment of the compounds of formula IG, L² is —CH₂—, R¹ and R⁵ are independently H or alkyl, and X is substituted or unsubstituted purinyl.

In another embodiment of the compounds of formula IG, L² is —CH(CH₃)—, R¹ and R⁵ are independently H or alkyl, and X is substituted or unsubstituted purinyl.

In another embodiment of the compounds of formula IG, L² is —C(O)—, R¹ and R⁵ are independently H or alkyl, and X is substituted or unsubstituted purinyl.

In another embodiment of the compounds of formula IG, L² is a covalent bond, R¹ and R⁵ are independently H or methyl, and X is substituted or unsubstituted purinyl.

In another embodiment of the compounds of formula IG, L² is —C(R⁴)₂—, R¹ and R⁵ are independently H or methyl, and X is substituted or unsubstituted purinyl.

In another embodiment of the compounds of formula IG, L² is —CH₂—, R¹ and R⁵ are independently H or methyl, and X is substituted or unsubstituted purinyl.

In another embodiment of the compounds of formula IG, L² is —CH(CH₃)—, R¹ and R⁵ are independently H or methyl, and X is substituted or unsubstituted purinyl.

In another embodiment of the compounds of formula IG, L² is —C(O)—, R¹ and R⁵ are independently H or methyl, and X is substituted or unsubstituted purinyl.

In another embodiment of the compounds of formula IG, L² is a covalent bond, R¹ and R⁵ are independently H or substituted or unsubstituted arylalkyl, and X is substituted or unsubstituted purinyl.

In another embodiment of the compounds of formula IG, L² is —C(R⁴)₂—, R¹ and R⁵ are independently H or substituted or unsubstituted arylalkyl, and X is substituted or unsubstituted purinyl.

In another embodiment of the compounds of formula IG, L² is —CH₂—, R¹ and R⁵ are independently H or substituted or unsubstituted arylalkyl, and X is substituted or unsubstituted purinyl.

In another embodiment of the compounds of formula IG, L² is —CH(CH₃)—, R¹ and R⁵ are independently H or substituted or unsubstituted arylalkyl, and X is substituted or unsubstituted purinyl.

In another embodiment of the compounds of formula IG, L² is —C(O)—, R¹ and R⁵ are independently H or substituted or unsubstituted arylalkyl, and X is substituted or unsubstituted purinyl.

In another embodiment of the compounds of formula IG, L² is a covalent bond, R¹ and R⁵ are both H, and X is substituted or unsubstituted purinyl.

In another embodiment of the compounds of formula IG, L² is —C(R⁴)₂—, R¹ and R⁵ are both H, and X is substituted or unsubstituted purinyl.

In another embodiment of the compounds of formula IG, L² is —CH₂—, R¹ and R⁵ are both H, and X is substituted or unsubstituted purinyl.

In another embodiment of the compounds of formula IG, L² is —CH(CH₃)—, R¹ and R⁵ are both H, and X is substituted or unsubstituted purinyl.

In another embodiment of the compounds of formula IG, L² is —C(O)—, R¹ and R⁵ are both H, and X is substituted or unsubstituted purinyl.

In another embodiment of the compounds of formula IG, L² is a covalent bond, R¹ and R⁵, together with the nitrogen atoms to which they are shown attached, form a 5-membered heterocyclyl ring, and X is substituted or unsubstituted purinyl.

In another embodiment of the compounds of formula IG, L² is —C(R⁴)₂—, R¹ and R⁵, together with the nitrogen atoms to which they are shown attached, form a 5-membered heterocyclyl ring, and X is substituted or unsubstituted purinyl.

In another embodiment of the compounds of formula IG, L² is —CH₂—, R¹ and R⁵, together with the nitrogen atoms to which they are shown attached, form a 5-membered heterocyclyl ring, and X is substituted or unsubstituted purinyl.

In another embodiment of the compounds of formula IG, L² is —CH(CH₃)—, R¹ and R⁵, together with the nitrogen atoms to which they are shown attached, form a 5-membered heterocyclyl ring, and X is substituted or unsubstituted purinyl.

In another embodiment of the compounds of formula IG, L² is —C(O)—, R¹ and R⁵, together with the nitrogen atoms to which they are shown attached, form a 5-membered heterocyclyl ring, and X is substituted or unsubstituted purinyl.

In another embodiment of the compounds of formula IG, L² is a covalent bond, R¹ and R⁵, together with the nitrogen atoms to which they are shown attached, form a 6-membered heterocyclyl ring, and X is substituted or unsubstituted purinyl.

In another embodiment of the compounds of formula IG, L² is —C(R⁴)₂—, R¹ and R⁵, together with the nitrogen atoms to which they are shown attached, form a 6-membered heterocyclyl ring, and X is substituted or unsubstituted purinyl.

In another embodiment of the compounds of formula IG, L² is —CH₂—, R¹ and R⁵, together with the nitrogen atoms to which they are shown attached, form a 6-membered heterocyclyl ring, and X is substituted or unsubstituted purinyl.

In another embodiment of the compounds of formula IG, L² is —CH(CH₃)—, R¹ and R⁵, together with the nitrogen atoms to which they are shown attached, form a 6-membered heterocyclyl ring, and X is substituted or unsubstituted purinyl.

In another embodiment of the compounds of formula IG, L² is —C(O)—, R¹ and R⁵, together with the nitrogen atoms to which they are shown attached, form a 6-membered heterocyclyl ring, and X is substituted or unsubstituted purinyl.

In another embodiment of the compounds of formula IG, L² is a covalent bond, R¹ and R⁵ are independently H or alkyl, and X is substituted or unsubstituted thienyl.

In another embodiment of the compounds of formula IG, L² is —C(R⁴)₂—, R¹ and R⁵ are independently H or alkyl, and X is substituted or unsubstituted thienyl.

In another embodiment of the compounds of formula IG, L² is —CH₂—, R¹ and R⁵ are independently H or alkyl, and X is substituted or unsubstituted thienyl.

In another embodiment of the compounds of formula IG, L² is —CH(CH₃)—, R¹ and R⁵ are independently H or alkyl, and X is substituted or unsubstituted thienyl.

In another embodiment of the compounds of formula IG, L² is —C(O)—, R¹ and R⁵ are independently H or alkyl, and X is substituted or unsubstituted thienyl.

In another embodiment of the compounds of formula IG, L² is a covalent bond, R¹ and R⁵ are independently H or methyl, and X is substituted or unsubstituted thienyl.

In another embodiment of the compounds of formula IG, L² is —C(R⁴)₂—, R¹ and R⁵ are independently H or methyl, and X is substituted or unsubstituted thienyl.

In another embodiment of the compounds of formula IG, L² is —CH₂—, R¹ and R⁵ are independently H or methyl, and X is substituted or unsubstituted thienyl.

In another embodiment of the compounds of formula IG, L² is —CH(CH₃)—, R¹ and R⁵ are independently H or methyl, and X is substituted or unsubstituted thienyl.

In another embodiment of the compounds of formula IG, L² is —C(O)—, R¹ and R⁵ are independently H or methyl, and X is substituted or unsubstituted thienyl.

In another embodiment of the compounds of formula IG, L² is a covalent bond, R¹ and R⁵ are independently H or substituted or unsubstituted arylalkyl, and X is substituted or unsubstituted thienyl.

In another embodiment of the compounds of formula IG, L² is —C(R⁴)₂—, R¹ and R⁵ are independently H or substituted or unsubstituted arylalkyl, and X is substituted or unsubstituted thienyl.

In another embodiment of the compounds of formula IG, L² is —CH₂—, R¹ and R⁵ are independently H or substituted or unsubstituted arylalkyl, and X is substituted or unsubstituted thienyl.

In another embodiment of the compounds of formula IG, L² is —CH(CH₃)—, R¹ and R⁵ are independently H or substituted or unsubstituted arylalkyl, and X is substituted or unsubstituted thienyl.

In another embodiment of the compounds of formula IG, L² is —C(O)—, R¹ and R⁵ are independently H or substituted or unsubstituted arylalkyl, and X is substituted or unsubstituted thienyl.

In another embodiment of the compounds of formula IG, L² is a covalent bond, R¹ and R⁵ are both H, and X is substituted or unsubstituted thienyl.

In another embodiment of the compounds of formula IG, L² is —C(R⁴)₂—, R¹ and R⁵ are both H, and X is substituted or unsubstituted thienyl.

In another embodiment of the compounds of formula IG, L² is —CH₂—, R¹ and R⁵ are both H, and X is substituted or unsubstituted thienyl.

In another embodiment of the compounds of formula IG, L² is —CH(CH₃)—, R¹ and R⁵ are both H, and X is substituted or unsubstituted thienyl.

In another embodiment of the compounds of formula IG, L² is —C(O)—, R¹ and R⁵ are both H, and X is substituted or unsubstituted thienyl.

In another embodiment of the compounds of formula IG, L² is a covalent bond, R¹ and R⁵, together with the nitrogen atoms to which they are shown attached, form a 5-membered heterocyclyl ring, and X is substituted or unsubstituted thienyl.

In another embodiment of the compounds of formula IG, L² is —C(R⁴)₂—, R¹ and R⁵, together with the nitrogen atoms to which they are shown attached, form a 5-membered heterocyclyl ring, and X is substituted or unsubstituted thienyl.

In another embodiment of the compounds of formula IG, L² is —CH₂—, R¹ and R⁵, together with the nitrogen atoms to which they are shown attached, form a 5-membered heterocyclyl ring, and X is substituted or unsubstituted thienyl.

In another embodiment of the compounds of formula IG, L² is —CH(CH₃)—, R¹ and R⁵, together with the nitrogen atoms to which they are shown attached, form a 5-membered heterocyclyl ring, and X is substituted or unsubstituted thienyl.

In another embodiment of the compounds of formula IG, L² is —C(O)—, R¹ and R⁵, together with the nitrogen atoms to which they are shown attached, form a 5-membered heterocyclyl ring, and X is substituted or unsubstituted thienyl.

In another embodiment of the compounds of formula IG, L² is a covalent bond, R¹ and R⁵, together with the nitrogen atoms to which they are shown attached, form a 6-membered heterocyclyl ring, and X is substituted or unsubstituted thienyl.

In another embodiment of the compounds of formula IG, L² is —C(R⁴)₂—, R¹ and R⁵, together with the nitrogen atoms to which they are shown attached, form a 6-membered heterocyclyl ring, and X is substituted or unsubstituted thienyl.

In another embodiment of the compounds of formula IG, L² is —CH₂—, R¹ and R⁵, together with the nitrogen atoms to which they are shown attached, form a 6-membered heterocyclyl ring, and X is substituted or unsubstituted thienyl.

In another embodiment of the compounds of formula IG, L² is —CH(CH₃)—, R¹ and R⁵, together with the nitrogen atoms to which they are shown attached, form a 6-membered heterocyclyl ring, and X is substituted or unsubstituted thienyl.

In another embodiment of the compounds of formula IG, L² is —C(O)—, R¹ and R⁵, together with the nitrogen atoms to which they are shown attached, form a 6-membered heterocyclyl ring, and X is substituted or unsubstituted thienyl.

In another embodiment of the compounds of formula IG, L² is a covalent bond, R¹ and R⁵ are independently H or alkyl, and X is substituted or unsubstituted furanyl.

In another embodiment of the compounds of formula IG, L² is —C(R⁴)₂—, R¹ and R⁵ are independently H or alkyl, and X is substituted or unsubstituted furanyl.

In another embodiment of the compounds of formula IG, L² is —CH₂—, R¹ and R⁵ are independently H or alkyl, and X is substituted or unsubstituted furanyl.

In another embodiment of the compounds of formula IG, L² is —CH(CH₃)—, R¹ and R⁵ are independently H or alkyl, and X is substituted or unsubstituted furanyl.

In another embodiment of the compounds of formula IG, L² is —C(O)—, R¹ and R⁵ are independently H or alkyl, and X is substituted or unsubstituted furanyl.

In another embodiment of the compounds of formula IG, L² is a covalent bond, R¹ and R⁵ are independently H or methyl, and X is substituted or unsubstituted furanyl.

In another embodiment of the compounds of formula IG, L² is —C(R⁴)₂—, R¹ and R⁵ are independently H or methyl, and X is substituted or unsubstituted furanyl.

In another embodiment of the compounds of formula IG, L² is —CH₂—, R¹ and R⁵ are independently H or methyl, and X is substituted or unsubstituted furanyl.

In another embodiment of the compounds of formula IG, L² is —CH(CH₃)—, R¹ and R⁵ are independently H or methyl, and X is substituted or unsubstituted furanyl.

In another embodiment of the compounds of formula IG, L² is —C(O)—, R¹ and R⁵ are independently H or methyl, and X is substituted or unsubstituted furanyl.

In another embodiment of the compounds of formula IG, L² is a covalent bond, R¹ and R⁵ are independently H or substituted or unsubstituted arylalkyl, and X is substituted or unsubstituted furanyl.

In another embodiment of the compounds of formula IG, L² is —C(R⁴)₂—, R¹ and R⁵ are independently H or substituted or unsubstituted arylalkyl, and X is substituted or unsubstituted furanyl.

In another embodiment of the compounds of formula IG, L² is —CH₂—, R¹ and R⁵ are independently H or substituted or unsubstituted arylalkyl, and X is substituted or unsubstituted furanyl.

In another embodiment of the compounds of formula IG, L² is —CH(CH₃)—, R¹ and R⁵ are independently H or substituted or unsubstituted arylalkyl, and X is substituted or unsubstituted furanyl.

In another embodiment of the compounds of formula IG, L² is —C(O)—, R¹ and R⁵ are independently H or substituted or unsubstituted arylalkyl, and X is substituted or unsubstituted furanyl.

In another embodiment of the compounds of formula IG, L² is a covalent bond, R¹ and R⁵ are both H, and X is substituted or unsubstituted furanyl.

In another embodiment of the compounds of formula IG, L² is —C(R⁴)₂—, R¹ and R⁵ are both H, and X is substituted or unsubstituted furanyl.

In another embodiment of the compounds of formula IG, L² is —CH₂—, R¹ and R⁵ are both H, and X is substituted or unsubstituted furanyl.

In another embodiment of the compounds of formula IG, L² is —CH(CH₃)—, R¹ and R⁵ are both H, and X is substituted or unsubstituted furanyl.

In another embodiment of the compounds of formula IG, L² is —C(O)—, R¹ and R⁵ are both H, and X is substituted or unsubstituted furanyl.

In another embodiment of the compounds of formula IG, L² is a covalent bond, R¹ and R⁵, together with the nitrogen atoms to which they are shown attached, form a 5-membered heterocyclyl ring, and X is substituted or unsubstituted furanyl.

In another embodiment of the compounds of formula IG, L² is —C(R⁴)₂—, R¹ and R⁵, together with the nitrogen atoms to which they are shown attached, form a 5-membered heterocyclyl ring, and X is substituted or unsubstituted furanyl.

In another embodiment of the compounds of formula IG, L² is —CH₂—, R¹ and R⁵, together with the nitrogen atoms to which they are shown attached, form a 5-membered heterocyclyl ring, and X is substituted or unsubstituted furanyl.

In another embodiment of the compounds of formula IG, L² is —CH(CH₃)—, R¹ and R⁵, together with the nitrogen atoms to which they are shown attached, form a 5-membered heterocyclyl ring, and X is substituted or unsubstituted furanyl.

In another embodiment of the compounds of formula IG, L² is —C(O)—, R¹ and R⁵, together with the nitrogen atoms to which they are shown attached, form a 5-membered heterocyclyl ring, and X is substituted or unsubstituted furanyl.

In another embodiment of the compounds of formula IG, L² is a covalent bond, R¹ and R⁵, together with the nitrogen atoms to which they are shown attached, form a 6-membered heterocyclyl ring, and X is substituted or unsubstituted furanyl.

In another embodiment of the compounds of formula IG, L² is —C(R⁴)₂—, R¹ and R⁵, together with the nitrogen atoms to which they are shown attached, form a 6-membered heterocyclyl ring, and X is substituted or unsubstituted furanyl.

In another embodiment of the compounds of formula IG, L² is —CH₂—, R¹ and R⁵, together with the nitrogen atoms to which they are shown attached, form a 6-membered heterocyclyl ring, and X is substituted or unsubstituted furanyl.

In another embodiment of the compounds of formula IG, L² is —CH(CH₃)—, R¹ and R⁵, together with the nitrogen atoms to which they are shown attached, form a 6-membered heterocyclyl ring, and X is substituted or unsubstituted furanyl.

In another embodiment of the compounds of formula IG, L² is —C(O)—, R¹ and R⁵, together with the nitrogen atoms to which they are shown attached, form a 6-membered heterocyclyl ring, and X is substituted or unsubstituted furanyl.

In another embodiment of the compounds of formula IG, L² is a covalent bond, R¹ and R⁵ are independently H or alkyl, and X is substituted or unsubstituted pyrrolyl.

In another embodiment of the compounds of formula IG, L² is —C(R⁴)₂—, R¹ and R⁵ are independently H or alkyl, and X is substituted or unsubstituted pyrrolyl.

In another embodiment of the compounds of formula IG, L² is —CH₂—, R¹ and R⁵ are independently H or alkyl, and X is substituted or unsubstituted pyrrolyl.

In another embodiment of the compounds of formula IG, L² is —CH(CH₃)—, R¹ and R⁵ are independently H or alkyl, and X is substituted or unsubstituted pyrrolyl.

In another embodiment of the compounds of formula IG, L² is —C(O)—, R¹ and R⁵ are independently H or alkyl, and X is substituted or unsubstituted pyrrolyl.

In another embodiment of the compounds of formula IG, L² is a covalent bond, R¹ and R⁵ are independently H or methyl, and X is substituted or unsubstituted pyrrolyl.

In another embodiment of the compounds of formula IG, L² is —C(R⁴)₂—, R¹ and R⁵ are independently H or methyl, and X is substituted or unsubstituted pyrrolyl.

In another embodiment of the compounds of formula IG, L² is —CH₂—, R¹ and R⁵ are independently H or methyl, and X is substituted or unsubstituted pyrrolyl.

In another embodiment of the compounds of formula IG, L² is —CH(CH₃)—, R¹ and R⁵ are independently H or methyl, and X is substituted or unsubstituted pyrrolyl.

In another embodiment of the compounds of formula IG, L² is —C(O)—, R¹ and R⁵ are independently H or methyl, and X is substituted or unsubstituted pyrrolyl.

In another embodiment of the compounds of formula IG, L² is a covalent bond, R¹ and R⁵ are independently H or substituted or unsubstituted arylalkyl, and X is substituted or unsubstituted pyrrolyl.

In another embodiment of the compounds of formula IG, L² is —C(R⁴)₂—, R¹ and R⁵ are independently H or substituted or unsubstituted arylalkyl, and X is substituted or unsubstituted pyrrolyl.

In another embodiment of the compounds of formula IG, L² is —CH₂—, R¹ and R⁵ are independently H or substituted or unsubstituted arylalkyl, and X is substituted or unsubstituted pyrrolyl.

In another embodiment of the compounds of formula IG, L² is —CH(CH₃)—, R¹ and R⁵ are independently H or substituted or unsubstituted arylalkyl, and X is substituted or unsubstituted pyrrolyl.

In another embodiment of the compounds of formula IG, L² is —C(O)—, R¹ and R⁵ are independently H or substituted or unsubstituted arylalkyl, and X is substituted or unsubstituted pyrrolyl.

In another embodiment of the compounds of formula IG, L² is a covalent bond, R¹ and R⁵ are both H, and X is substituted or unsubstituted pyrrolyl.

In another embodiment of the compounds of formula IG, L² is —C(R⁴)₂—, R¹ and R⁵ are both H, and X is substituted or unsubstituted pyrrolyl.

In another embodiment of the compounds of formula IG, L² is —CH₂—, R¹ and R⁵ are both H, and X is substituted or unsubstituted pyrrolyl.

In another embodiment of the compounds of formula IG, L² is —CH(CH₃)—, R¹ and R⁵ are both H, and X is substituted or unsubstituted pyrrolyl.

In another embodiment of the compounds of formula IG, L² is —C(O)—, R¹ and R⁵ are both H, and X is substituted or unsubstituted pyrrolyl.

In another embodiment of the compounds of formula IG, L² is a covalent bond, R¹ and R⁵, together with the nitrogen atoms to which they are shown attached, form a 5-membered heterocyclyl ring, and X is substituted or unsubstituted pyrrolyl.

In another embodiment of the compounds of formula IG, L² is —C(R⁴)₂—, R¹ and R⁵, together with the nitrogen atoms to which they are shown attached, form a 5-membered heterocyclyl ring, and X is substituted or unsubstituted pyrrolyl.

In another embodiment of the compounds of formula IG, L² is —CH₂—, R¹ and R⁵, together with the nitrogen atoms to which they are shown attached, form a 5-membered heterocyclyl ring, and X is substituted or unsubstituted pyrrolyl.

In another embodiment of the compounds of formula IG, L² is —CH(CH₃)—, R¹ and R⁵, together with the nitrogen atoms to which they are shown attached, form a 5-membered heterocyclyl ring, and X is substituted or unsubstituted pyrrolyl.

In another embodiment of the compounds of formula IG, L² is —C(O)—, R¹ and R⁵, together with the nitrogen atoms to which they are shown attached, form a 5-membered heterocyclyl ring, and X is substituted or unsubstituted pyrrolyl.

In another embodiment of the compounds of formula IG, L² is a covalent bond, R¹ and R⁵, together with the nitrogen atoms to which they are shown attached, form a 6-membered heterocyclyl ring, and X is substituted or unsubstituted pyrrolyl.

In another embodiment of the compounds of formula IG, L² is —C(R⁴)₂—, R¹ and R⁵, together with the nitrogen atoms to which they are shown attached, form a 6-membered heterocyclyl ring, and X is substituted or unsubstituted pyrrolyl.

In another embodiment of the compounds of formula IG, L² is —CH₂—, R¹ and R⁵, together with the nitrogen atoms to which they are shown attached, form a 6-membered heterocyclyl ring, and X is substituted or unsubstituted pyrrolyl.

In another embodiment of the compounds of formula IG, L² is —CH(CH₃)—, R¹ and R⁵, together with the nitrogen atoms to which they are shown attached, form a 6-membered heterocyclyl ring, and X is substituted or unsubstituted pyrrolyl.

In another embodiment of the compounds of formula IG, L³ is —C(O)—, R¹ and R⁵, together with the nitrogen atoms to which they are shown attached, form a 6-membered heterocyclyl ring, and X is substituted or unsubstituted pyrrolyl.

In another embodiment of the compounds of formula IG, L³ is —C(R⁴)₂—.

In another embodiment of the compounds of formula IG, L³ is —CH₂—.

In another embodiment of the compounds of formula IG, L³ is —CH(CH₃)—.

In another embodiment of the compounds of formula IG, L³ is —C(O)—.

In another embodiment of the compounds of formula IG, L³ is —S(O)₂—.

In another embodiment of the compounds of formula IG, L⁴ is a covalent bond.

In another embodiment of the compounds of formula IG, L⁴ is —C(R⁴)₂—.

In another embodiment of the compounds of formula IG, L⁴ is —CH₂—.

In another embodiment of the compounds of formula IG, L⁴ is —CH(CH₃)—.

In another embodiment of the compounds of formula IG, L⁴ is —C(O)—.

In another embodiment of the compounds of formula IG, L⁴ is —S(O)₂—.

In another embodiment of the compounds of formula IG, L³ is —C(R⁴)₂—, and L⁴ is a covalent bond.

In another embodiment of the compounds of formula IG, L³ is —CH₂—, and L⁴ is a covalent bond.

In another embodiment of the compounds of formula IG, L³ is —CH(CH₃)—, and L⁴ is a covalent bond.

In another embodiment of the compounds of formula IG, L³ is —C(O)—, and L⁴ is a covalent bond.

In another embodiment of the compounds of formula IG, L³ is —S(O)₂—, and L⁴ is a covalent bond.

In another embodiment of the compounds of formula IG, L³ is —C(R⁴)₂—, and L⁴ is —C(R⁴)₂—.

In another embodiment of the compounds of formula IG, L³ is —CH₂—, and L⁴ is —C(R⁴)₂—.

In another embodiment of the compounds of formula IG, L³ is —CH(CH₃)—, and L⁴ is —C(R⁴)₂—.

In another embodiment of the compounds of formula IG, L³ is —C(O)—, and L⁴ is —C(R⁴)₂—.

In another embodiment of the compounds of formula IG, L³ is —S(O)₂—, and L⁴ is —C(R⁴)₂—.

In another embodiment of the compounds of formula IG, L³ is —C(R⁴)₂—, and L⁴ is —CH₂—.

In another embodiment of the compounds of formula IG, L³ is —CH₂—, and L⁴ is —CH₂—.

In another embodiment of the compounds of formula IG, L³ is —CH(CH₃)—, and L⁴ is —CH₂—.

In another embodiment of the compounds of formula IG, L³ is —C(O)—, and L⁴ is —CH₂—.

In another embodiment of the compounds of formula IG, L³ is —S(O)₂—, and L⁴ is —CH₂—.

In another embodiment of the compounds of formula IG, L³ is —C(R⁴)₂—, and L⁴ is —CH(CH₃)—.

In another embodiment of the compounds of formula IG, L³ is —CH₂—, and L⁴ is —CH(CH₃)—.

In another embodiment of the compounds of formula IG, L³ is —CH(CH₃)—, and L⁴ is —CH(CH₃)—.

In another embodiment of the compounds of formula IG, L³ is —C(O)—, and L⁴ is —CH(CH₃)—.

In another embodiment of the compounds of formula IG, L³ is —S(O)₂—, and L⁴ is —CH(CH₃)—.

In another embodiment of the compounds of formula IG, L³ is —C(R⁴)₂—, and L⁴ is —C(O)—

In another embodiment of the compounds of formula IG, L³ is —CH₂—, and L⁴ is —C(O)—.

In another embodiment of the compounds of formula IG, L³ is —CH(CH₃)—, and L⁴ is —C(O)—.

In another embodiment of the compounds of formula IG, L³ is —C(O)—, and L⁴ is —C(O)—.

In another embodiment of the compounds of formula IG, L³ is —S(O)₂—, and L⁴ is —C(O)—.

In another embodiment of the compounds of formula IG, L³ is —C(R⁴)₂—, and L⁴ is —S(O)₂—.

In another embodiment of the compounds of formula IG, L³ is —CH₂—, and L⁴ is —S(O)₂—.

In another embodiment of the compounds of formula IG, L³ is —CH(CH₃)—, and L⁴ is —S(O)₂—.

In another embodiment of the compounds of formula IG, L³ is —C(O)—, and L⁴ is —S(O)₂—

In another embodiment of the compounds of formula IG, L³ is —S(O)₂—, and L⁴ is —S(O)₂—

In another embodiment of the compounds of formula IG, R⁶ is H.

In another embodiment of the compounds of formula IG, R⁶ is substituted or unsubstituted arylalkyl.

In another embodiment of the compounds of formula IG, R⁶ is substituted or unsubstituted benzyl.

In another embodiment of the compounds of formula IG, R⁶ is substituted or unsubstituted cycloalkyl.

In another embodiment of the compounds of formula IG, R⁶ is substituted or unsubstituted cyclopentyl.

In another embodiment of the compounds of formula IG, R⁶ is substituted or unsubstituted cyclohexyl.

In another embodiment of the compounds of formula IG, R⁶ is substituted or unsubstituted tetrahydronaphthyl.

In another embodiment of the compounds of formula IG, L³ is —C(R⁴)₂—, L⁴ is a covalent bond, and Y is substituted or unsubstituted aryl.

In another embodiment of the compounds of formula IG, L³ is —CH₂—, L⁴ is a covalent bond, and Y is substituted or unsubstituted aryl.

In another embodiment of the compounds of formula IG, L³ is —CH(CH₃)—, L⁴ is a covalent bond, and Y is substituted or unsubstituted aryl.

In another embodiment of the compounds of formula IG, L³ is —C(O)—, L⁴ is a covalent bond, and Y is substituted or unsubstituted aryl.

In another embodiment of the compounds of formula IG, L³ is —S(O)₂—, L⁴ is a covalent bond, and Y is substituted or unsubstituted aryl.

In another embodiment of the compounds of formula IG, L³ is —C(R⁴)₂—, L⁴ is —C(R⁴)₂—, and Y is substituted or unsubstituted aryl.

In another embodiment of the compounds of formula IG, L³ is —CH₂—, L⁴ is —C(R⁴)₂—, and Y is substituted or unsubstituted aryl.

In another embodiment of the compounds of formula IG, L³ is —CH(CH₃)—, L⁴ is —C(R⁴)₂—, and Y is substituted or unsubstituted aryl.

In another embodiment of the compounds of formula IG, L³ is —C(O)—, L⁴ is —C(R⁴)₂—, and Y is substituted or unsubstituted aryl.

In another embodiment of the compounds of formula IG, L³ is —S(O)₂—, L⁴ is —C(R⁴)₂—, and Y is substituted or unsubstituted aryl.

In another embodiment of the compounds of formula IG, L³ is —C(R⁴)₂—, L⁴ is —CH₂—, and Y is substituted or unsubstituted aryl.

In another embodiment of the compounds of formula IG, L³ is —CH₂—, L⁴ is —CH₂—, and Y is substituted or unsubstituted aryl.

In another embodiment of the compounds of formula IG, L³ is —CH(CH₃)—, L⁴ is —CH₂—, and Y is substituted or unsubstituted aryl.

In another embodiment of the compounds of formula IG, L³ is —C(O)—, L⁴ is —CH₂—, and Y is substituted or unsubstituted aryl.

In another embodiment of the compounds of formula IG, L³ is —S(O)₂—, L⁴ is —CH₂—, and Y is substituted or unsubstituted aryl.

In another embodiment of the compounds of formula IG, L³ is —C(R⁴)₂—, L⁴ is —CH(CH₃)—, and Y is substituted or unsubstituted aryl.

In another embodiment of the compounds of formula IG, L³ is —CH₂—, L⁴ is —CH(CH₃)—, and Y is substituted or unsubstituted aryl.

In another embodiment of the compounds of formula IG, L³ is —CH(CH₃)—, L⁴ is —CH(CH₃)—, and Y is substituted or unsubstituted aryl.

In another embodiment of the compounds of formula IG, L³ is —C(O)—, L⁴ is —CH(CH₃)—, and Y is substituted or unsubstituted aryl.

In another embodiment of the compounds of formula IG, L³ is —S(O)₂—, L⁴ is —CH(CH₃)—, and Y is substituted or unsubstituted aryl.

In another embodiment of the compounds of formula IG, L³ is —C(R⁴)₂—, L⁴ is —C(O)—, and Y is substituted or unsubstituted aryl.

In another embodiment of the compounds of formula IG, L³ is —CH₂—, L⁴ is —C(O)—, and Y is substituted or unsubstituted aryl.

In another embodiment of the compounds of formula IG, L³ is —CH(CH₃)—, L⁴ is —C(O)—, and Y is substituted or unsubstituted aryl.

In another embodiment of the compounds of formula IG, L³ is —C(O)—, L⁴ is —C(O)—, and Y is substituted or unsubstituted aryl.

In another embodiment of the compounds of formula IG, L³ is —S(O)₂—, L⁴ is —C(O)—, and Y is substituted or unsubstituted aryl.

In another embodiment of the compounds of formula IG, L³ is —C(R⁴)₂—, L⁴ is —S(O)₂—, and Y is substituted or unsubstituted aryl.

In another embodiment of the compounds of formula IG, L³ is —CH₂—, L⁴ is —S(O)₂—, and Y is substituted or unsubstituted aryl.

In another embodiment of the compounds of formula IG, L³ is —CH(CH₃)—, L⁴ is —S(O)₂—, and Y is substituted or unsubstituted aryl.

In another embodiment of the compounds of formula IG, L³ is —C(O)—, L⁴ is —S(O)₂—, and Y is substituted or unsubstituted aryl.

In another embodiment of the compounds of formula IG, L³ is —S(O)₂—, L⁴ is —S(O)₂—, and Y is substituted or unsubstituted aryl.

In another embodiment of the compounds of formula IG, L³ is —C(R⁴)₂—, L⁴ is a covalent bond, and Y is substituted or unsubstituted phenyl.

In another embodiment of the compounds of formula IG, L³ is —CH₂—, L⁴ is a covalent bond, and Y is substituted or unsubstituted phenyl.

In another embodiment of the compounds of formula IG, L³ is —CH(CH₃)—, L⁴ is a covalent bond, and Y is substituted or unsubstituted phenyl.

In another embodiment of the compounds of formula IG, L³ is —C(O)—, L⁴ is a covalent bond, and Y is substituted or unsubstituted phenyl.

In another embodiment of the compounds of formula IG, L³ is —S(O)₂—, L⁴ is a covalent bond, and Y is substituted or unsubstituted phenyl.

In another embodiment of the compounds of formula IG, L³ is —C(R⁴)₂—, L⁴ is —C(R⁴)₂—, and Y is substituted or unsubstituted phenyl.

In another embodiment of the compounds of formula IG, L³ is —CH₂—, L⁴ is —C(R⁴)₂—, and Y is substituted or unsubstituted phenyl.

In another embodiment of the compounds of formula IG, L³ is —CH(CH₃)—, L⁴ is —C(R⁴)₂—, and Y is substituted or unsubstituted phenyl.

In another embodiment of the compounds of formula IG, L³ is —C(O)—, L⁴ is —C(R⁴)₂—, and Y is substituted or unsubstituted phenyl.

In another embodiment of the compounds of formula IG, L³ is —S(O)₂—, L⁴ is —C(R⁴)₂—, and Y is substituted or unsubstituted phenyl.

In another embodiment of the compounds of formula IG, L³ is —C(R⁴)₂—, L⁴ is —CH₂—, and Y is substituted or unsubstituted phenyl.

In another embodiment of the compounds of formula IG, L³ is —CH₂—, L⁴ is —CH₂—, and Y is substituted or unsubstituted phenyl.

In another embodiment of the compounds of formula IG, L³ is —CH(CH₃)—, L⁴ is —CH₂—, and Y is substituted or unsubstituted phenyl.

In another embodiment of the compounds of formula IG, L³ is —C(O)—, L⁴ is —CH₂—, and Y is substituted or unsubstituted phenyl.

In another embodiment of the compounds of formula IG, L³ is —S(O)₂—, L⁴ is —CH₂—, and Y is substituted or unsubstituted phenyl.

In another embodiment of the compounds of formula IG, L³ is —C(R⁴)₂—, L⁴ is —CH(CH₃)—, and Y is substituted or unsubstituted phenyl.

In another embodiment of the compounds of formula IG, L³ is —CH₂—, L⁴ is —CH(CH₃)—, and Y is substituted or unsubstituted phenyl.

In another embodiment of the compounds of formula IG, L³ is —CH(CH₃)—, L⁴ is —CH(CH₃)—, and Y is substituted or unsubstituted phenyl.

In another embodiment of the compounds of formula IG, L³ is —C(O)—, L⁴ is —CH(CH₃)—, and Y is substituted or unsubstituted phenyl.

In another embodiment of the compounds of formula IG, L³ is —S(O)₂—, L⁴ is —CH(CH₃)—, and Y is substituted or unsubstituted phenyl.

In another embodiment of the compounds of formula IG, L³ is —C(R⁴)₂—, L⁴ is —C(O)—, and Y is substituted or unsubstituted phenyl.

In another embodiment of the compounds of formula IG, L³ is —CH₂—, L⁴ is —C(O)—, and Y is substituted or unsubstituted phenyl.

In another embodiment of the compounds of formula IG, L³ is —CH(CH₃)—, L⁴ is —C(O)—, and Y is substituted or unsubstituted phenyl.

In another embodiment of the compounds of formula IG, L³ is —C(O)—, L⁴ is —C(O)—, and Y is substituted or unsubstituted phenyl.

In another embodiment of the compounds of formula IG, L³ is —S(O)₂—, L⁴ is —C(O)—, and Y is substituted or unsubstituted phenyl.

In another embodiment of the compounds of formula IG, L³ is —C(R⁴)₂—, L⁴ is —S(O)₂—, and Y is substituted or unsubstituted phenyl.

In another embodiment of the compounds of formula IG, L³ is —CH₂—, L⁴ is —S(O)₂—, and Y is substituted or unsubstituted phenyl.

In another embodiment of the compounds of formula IG, L³ is —CH(CH₃)—, L⁴ is —S(O)₂—, and Y is substituted or unsubstituted phenyl.

In another embodiment of the compounds of formula IG, L³ is —C(O)—, L⁴ is —S(O)₂—, and Y is substituted or unsubstituted phenyl.

In another embodiment of the compounds of formula IG, L³ is —S(O)₂—, L⁴ is —S(O)₂—, and Y is substituted or unsubstituted phenyl.

In another embodiment of the compounds of formula IG, L³ is —C(R⁴)₂—, L⁴ is a covalent bond, and Y is substituted or unsubstituted heteroaryl.

In another embodiment of the compounds of formula IG, L³ is —CH₂—, L⁴ is a covalent bond, and Y is substituted or unsubstituted heteroaryl.

In another embodiment of the compounds of formula IG, L³ is —CH(CH₃)—, L⁴ is a covalent bond, and Y is substituted or unsubstituted heteroaryl.

In another embodiment of the compounds of formula IG, L³ is —C(O)—, L⁴ is a covalent bond, and Y is substituted or unsubstituted heteroaryl.

In another embodiment of the compounds of formula IG, L³ is —S(O)₂—, L⁴ is a covalent bond, and Y is substituted or unsubstituted heteroaryl.

In another embodiment of the compounds of formula IG, L³ is —C(R⁴)₂—, L⁴ is —C(R⁴)₂—, and Y is substituted or unsubstituted heteroaryl.

In another embodiment of the compounds of formula IG, L³ is —CH₂—, L⁴ is —C(R⁴)₂—, and Y is substituted or unsubstituted heteroaryl.

In another embodiment of the compounds of formula IG, L³ is —CH(CH₃)—, L⁴ is —C(R⁴)₂—, and Y is substituted or unsubstituted heteroaryl.

In another embodiment of the compounds of formula IG, L³ is —C(O)—, L⁴ is —C(R⁴)₂—, and Y is substituted or unsubstituted heteroaryl.

In another embodiment of the compounds of formula IG, L³ is —S(O)₂—, L⁴ is —C(R⁴)₂—, and Y is substituted or unsubstituted heteroaryl.

In another embodiment of the compounds of formula IG, L³ is —C(R⁴)₂—, L⁴ is —CH₂—, and Y is substituted or unsubstituted heteroaryl.

In another embodiment of the compounds of formula IG, L³ is —CH₂—, L⁴ is —CH₂—, and Y is substituted or unsubstituted heteroaryl.

In another embodiment of the compounds of formula IG, L³ is —CH(CH₃)—, L⁴ is —CH₂—, and Y is substituted or unsubstituted heteroaryl.

In another embodiment of the compounds of formula IG, L³ is —C(O)—, L⁴ is —CH₂—, and Y is substituted or unsubstituted heteroaryl.

In another embodiment of the compounds of formula IG, L³ is —S(O)₂—, L⁴ is —CH₂—, and Y is substituted or unsubstituted heteroaryl.

In another embodiment of the compounds of formula IG, L³ is —C(R⁴)₂—, L⁴ is —CH(CH₃)—, and Y is substituted or unsubstituted heteroaryl.

In another embodiment of the compounds of formula IG, L³ is —CH₂—, L⁴ is —CH(CH₃)—, and Y is substituted or unsubstituted heteroaryl.

In another embodiment of the compounds of formula IG, L³ is —CH(CH₃)—, L⁴ is —CH(CH₃)—, and Y is substituted or unsubstituted heteroaryl.

In another embodiment of the compounds of formula IG, L³ is —C(O)—, L⁴ is —CH(CH₃)—, and Y is substituted or unsubstituted heteroaryl.

In another embodiment of the compounds of formula IG, L³ is —S(O)₂—, L⁴ is —CH(CH₃)—, and Y is substituted or unsubstituted heteroaryl.

In another embodiment of the compounds of formula IG, L³ is —C(R⁴)₂—, L⁴ is —C(O)—, and Y is substituted or unsubstituted heteroaryl.

In another embodiment of the compounds of formula IG, L³ is —CH₂—, L⁴ is —C(O)—, and Y is substituted or unsubstituted heteroaryl.

In another embodiment of the compounds of formula IG, L³ is —CH(CH₃)—, L⁴ is —C(O)—, and Y is substituted or unsubstituted heteroaryl.

In another embodiment of the compounds of formula IG, L³ is —C(O)—, L⁴ is —C(O)—, and Y is substituted or unsubstituted heteroaryl.

In another embodiment of the compounds of formula IG, L³ is —S(O)₂—, L⁴ is —C(O)—, and Y is substituted or unsubstituted heteroaryl.

In another embodiment of the compounds of formula IG, L³ is —C(R⁴)₂—, L⁴ is —S(O)₂—, and Y is substituted or unsubstituted heteroaryl.

In another embodiment of the compounds of formula IG, L³ is —CH₂—, L⁴ is —S(O)₂—, and Y is substituted or unsubstituted heteroaryl.

In another embodiment of the compounds of formula IG, L³ is —CH(CH₃)—, L⁴ is —S(O)₂—, and Y is substituted or unsubstituted heteroaryl.

In another embodiment of the compounds of formula IG, L³ is —C(O)—, L⁴ is —S(O)₂—, and Y is substituted or unsubstituted heteroaryl.

In another embodiment of the compounds of formula IG, L³ is —S(O)₂—, L⁴ is —S(O)₂—, and Y is substituted or unsubstituted heteroaryl.

In another embodiment of the compounds of formula IG, L³ is —C(R⁴)₂—, L⁴ is a covalent bond, and Y is substituted or unsubstituted pyridyl.

In another embodiment of the compounds of formula IG, L³ is —CH₂—, L⁴ is a covalent bond, and Y is substituted or unsubstituted pyridyl.

In another embodiment of the compounds of formula IG, L³ is —CH(CH₃)—, L⁴ is a covalent bond, and Y is substituted or unsubstituted pyridyl.

In another embodiment of the compounds of formula IG, L³ is —C(O)—, L⁴ is a covalent bond, and Y is substituted or unsubstituted pyridyl.

In another embodiment of the compounds of formula IG, L³ is —S(O)₂—, L⁴ is a covalent bond, and Y is substituted or unsubstituted pyridyl.

In another embodiment of the compounds of formula IG, L³ is —C(R⁴)₂—, L⁴ is —C(R⁴)₂—, and Y is substituted or unsubstituted pyridyl.

In another embodiment of the compounds of formula IG, L³ is —CH₂—, L⁴ is —C(R⁴)₂—, and Y is substituted or unsubstituted pyridyl.

In another embodiment of the compounds of formula IG, L³ is —CH(CH₃)—, L⁴ is —C(R⁴)₂—, and Y is substituted or unsubstituted pyridyl.

In another embodiment of the compounds of formula IG, L³ is —C(O)—, L⁴ is —C(R⁴)₂—, and Y is substituted or unsubstituted pyridyl.

In another embodiment of the compounds of formula IG, L³ is —S(O)₂—, L⁴ is —C(R⁴)₂—, and Y is substituted or unsubstituted pyridyl.

In another embodiment of the compounds of formula IG, L³ is —C(R⁴)₂—, L⁴ is —CH₂—, and Y is substituted or unsubstituted pyridyl.

In another embodiment of the compounds of formula IG, L³ is —CH₂—, L⁴ is —CH₂—, and Y is substituted or unsubstituted pyridyl.

In another embodiment of the compounds of formula IG, L³ is —CH(CH₃)—, L⁴ is —CH₂—, and Y is substituted or unsubstituted pyridyl.

In another embodiment of the compounds of formula IG, L³ is —C(O)—, L⁴ is —CH₂—, and Y is substituted or unsubstituted pyridyl.

In another embodiment of the compounds of formula IG, L³ is —S(O)₂—, L⁴ is —CH₂—, and Y is substituted or unsubstituted pyridyl.

In another embodiment of the compounds of formula IG, L³ is —C(R⁴)₂—, L⁴ is —CH(CH₃)—, and Y is substituted or unsubstituted pyridyl.

In another embodiment of the compounds of formula IG, L³ is —CH₂—, L⁴ is —CH(CH₃)—, and Y is substituted or unsubstituted pyridyl.

In another embodiment of the compounds of formula IG, L³ is —CH(CH₃)—, L⁴ is —CH(CH₃)—, and Y is substituted or unsubstituted pyridyl.

In another embodiment of the compounds of formula IG, L³ is —C(O)—, L⁴ is —CH(CH₃)—, and Y is substituted or unsubstituted pyridyl.

In another embodiment of the compounds of formula IG, L³ is —S(O)₂—, L⁴ is —CH(CH₃)—, and Y is substituted or unsubstituted pyridyl.

In another embodiment of the compounds of formula IG, L³ is —C(R⁴)₂—, L⁴ is —C(O)—, and Y is substituted or unsubstituted pyridyl.

In another embodiment of the compounds of formula IG, L³ is —CH₂—, L⁴ is —C(O)—, and Y is substituted or unsubstituted pyridyl.

In another embodiment of the compounds of formula IG, L³ is —CH(CH₃)—, L⁴ is —C(O)—, and Y is substituted or unsubstituted pyridyl.

In another embodiment of the compounds of formula IG, L³ is —C(O)—, L⁴ is —C(O)—, and Y is substituted or unsubstituted pyridyl.

In another embodiment of the compounds of formula IG, L³ is —S(O)₂—, L⁴ is —C(O)—, and Y is substituted or unsubstituted pyridyl.

In another embodiment of the compounds of formula IG, L³ is —C(R⁴)₂—, L⁴ is —S(O)₂—, and Y is substituted or unsubstituted pyridyl.

In another embodiment of the compounds of formula IG, L³ is —CH₂—, L⁴ is —S(O)₂—, and Y is substituted or unsubstituted pyridyl.

In another embodiment of the compounds of formula IG, L³ is —CH(CH₃)—, L⁴ is —S(O)₂—, and Y is substituted or unsubstituted pyridyl.

In another embodiment of the compounds of formula IG, L³ is —C(O)—, L⁴ is —S(O)₂—, and Y is substituted or unsubstituted pyridyl.

In another embodiment of the compounds of formula IG, L³ is —S(O)₂—, L⁴ is —S(O)₂—, and Y is substituted or unsubstituted pyridyl.

In another embodiment of the compounds of formula IG, L³ is —C(R⁴)₂—, L⁴ is a covalent bond, and Y is substituted or unsubstituted pyrimidyl.

In another embodiment of the compounds of formula IG, L³ is —CH₂—, L⁴ is a covalent bond, and Y is substituted or unsubstituted pyrimidyl.

In another embodiment of the compounds of formula IG, L³ is —CH(CH₃)—, L⁴ is a covalent bond, and Y is substituted or unsubstituted pyrimidyl.

In another embodiment of the compounds of formula IG, L³ is —C(O)—, L⁴ is a covalent bond, and Y is substituted or unsubstituted pyrimidyl.

In another embodiment of the compounds of formula IG, L³ is —S(O)₂—, L⁴ is a covalent bond, and Y is substituted or unsubstituted pyrimidyl.

In another embodiment of the compounds of formula IG, L³ is —C(R⁴)₂—, L⁴ is —C(R⁴)₂—, and Y is substituted or unsubstituted pyrimidyl.

In another embodiment of the compounds of formula IG, L³ is —CH₂—, L⁴ is —C(R⁴)₂—, and Y is substituted or unsubstituted pyrimidyl.

In another embodiment of the compounds of formula IG, L³ is —CH(CH₃)—, L⁴ is —C(R⁴)₂—, and Y is substituted or unsubstituted pyrimidyl.

In another embodiment of the compounds of formula IG, L³ is —C(O)—, L⁴ is —C(R⁴)₂—, and Y is substituted or unsubstituted pyrimidyl.

In another embodiment of the compounds of formula IG, L³ is —S(O)₂—, L⁴ is —C(R⁴)₂—, and Y is substituted or unsubstituted pyrimidyl.

In another embodiment of the compounds of formula IG, L³ is —C(R⁴)₂—, L⁴ is —CH₂—, and Y is substituted or unsubstituted pyrimidyl.

In another embodiment of the compounds of formula IG, L³ is —CH₂—, L⁴ is —CH₂—, and Y is substituted or unsubstituted pyrimidyl.

In another embodiment of the compounds of formula IG, L³ is —CH(CH₃)—, L⁴ is —CH₂—, and Y is substituted or unsubstituted pyrimidyl.

In another embodiment of the compounds of formula IG, L³ is —C(O)—, L⁴ is —CH₂—, and Y is substituted or unsubstituted pyrimidyl.

In another embodiment of the compounds of formula IG, L³ is —S(O)₂—, L⁴ is —CH₂—, and Y is substituted or unsubstituted pyrimidyl.

In another embodiment of the compounds of formula IG, L³ is —C(R⁴)₂—, L⁴ is —CH(CH₃)—, and Y is substituted or unsubstituted pyrimidyl.

In another embodiment of the compounds of formula IG, L³ is —CH₂—, L⁴ is —CH(CH₃)—, and Y is substituted or unsubstituted pyrimidyl.

In another embodiment of the compounds of formula IG, L³ is —CH(CH₃)—, L⁴ is —CH(CH₃)—, and Y is substituted or unsubstituted pyrimidyl.

In another embodiment of the compounds of formula IG, L³ is —C(O)—, L⁴ is —CH(CH₃)—, and Y is substituted or unsubstituted pyrimidyl.

In another embodiment of the compounds of formula IG, L³ is —S(O)₂—, L⁴ is —CH(CH₃)—, and Y is substituted or unsubstituted pyrimidyl.

In another embodiment of the compounds of formula IG, L³ is —C(R⁴)₂—, L⁴ is —C(O)—, and Y is substituted or unsubstituted pyrimidyl.

In another embodiment of the compounds of formula IG, L³ is —CH₂—, L⁴ is —C(O)—, and Y is substituted or unsubstituted pyrimidyl.

In another embodiment of the compounds of formula IG, L³ is —CH(CH₃)—, L⁴ is —C(O)—, and Y is substituted or unsubstituted pyrimidyl.

In another embodiment of the compounds of formula IG, L³ is —C(O)—, L⁴ is —C(O)—, and Y is substituted or unsubstituted pyrimidyl.

In another embodiment of the compounds of formula IG, L³ is —S(O)₂—, L⁴ is —C(O)—, and Y is substituted or unsubstituted pyrimidyl.

In another embodiment of the compounds of formula IG, L³ is —C(R⁴)₂—, L⁴ is —S(O)₂—, and Y is substituted or unsubstituted pyrimidyl.

In another embodiment of the compounds of formula IG, L³ is —CH₂—, L⁴ is —S(O)₂—, and Y is substituted or unsubstituted pyrimidyl.

In another embodiment of the compounds of formula IG, L³ is —CH(CH₃)—, L⁴ is —S(O)₂—, and Y is substituted or unsubstituted pyrimidyl.

In another embodiment of the compounds of formula IG, L³ is —C(O)—, L⁴ is —S(O)₂—, and Y is substituted or unsubstituted pyrimidyl.

In another embodiment of the compounds of formula IG, L³ is —S(O)₂—, L⁴ is —S(O)₂—, and Y is substituted or unsubstituted pyrimidyl.

In another embodiment of the compounds of formula IG, L³ is —C(R⁴)₂—, L⁴ is a covalent bond, and Y is substituted or unsubstituted indolyl.

In another embodiment of the compounds of formula IG, L³ is —CH₂—, L⁴ is a covalent bond, and Y is substituted or unsubstituted indolyl.

In another embodiment of the compounds of formula IG, L³ is —CH(CH₃)—, L⁴ is a covalent bond, and Y is substituted or unsubstituted indolyl.

In another embodiment of the compounds of formula IG, L³ is —C(O)—, L⁴ is a covalent bond, and Y is substituted or unsubstituted indolyl.

In another embodiment of the compounds of formula IG, L³ is —S(O)₂—, L⁴ is a covalent bond, and Y is substituted or unsubstituted indolyl.

In another embodiment of the compounds of formula IG, L³ is —C(R⁴)₂—, L⁴ is —C(R⁴)₂—, and Y is substituted or unsubstituted indolyl.

In another embodiment of the compounds of formula IG, L³ is —CH₂—, L⁴ is —C(R⁴)₂—, and Y is substituted or unsubstituted indolyl.

In another embodiment of the compounds of formula IG, L³ is —CH(CH₃)—, L⁴ is —C(R⁴)₂—, and Y is substituted or unsubstituted indolyl.

In another embodiment of the compounds of formula IG, L³ is —C(O)—, L⁴ is —C(R⁴)₂—, and Y is substituted or unsubstituted indolyl.

In another embodiment of the compounds of formula IG, L³ is —S(O)₂—, L⁴ is —C(R⁴)₂—, and Y is substituted or unsubstituted indolyl.

In another embodiment of the compounds of formula IG, L³ is —C(R⁴)₂—, L⁴ is —CH₂—, and Y is substituted or unsubstituted indolyl.

In another embodiment of the compounds of formula IG, L³ is —CH₂—, L⁴ is —CH₂—, and Y is substituted or unsubstituted indolyl.

In another embodiment of the compounds of formula IG, L³ is —CH(CH₃)—, L⁴ is —CH₂—, and Y is substituted or unsubstituted indolyl.

In another embodiment of the compounds of formula IG, L³ is —C(O)—, L⁴ is —CH₂—, and Y is substituted or unsubstituted indolyl.

In another embodiment of the compounds of formula IG, L³ is —S(O)₂—, L⁴ is —CH₂—, and Y is substituted or unsubstituted indolyl.

In another embodiment of the compounds of formula IG, L³ is —C(R⁴)₂—, L⁴ is —CH(CH₃)—, and Y is substituted or unsubstituted indolyl.

In another embodiment of the compounds of formula IG, L³ is —CH₂—, L⁴ is —CH(CH₃)—, and Y is substituted or unsubstituted indolyl.

In another embodiment of the compounds of formula IG, L³ is —CH(CH₃)—, L⁴ is —CH(CH₃)—, and Y is substituted or unsubstituted indolyl.

In another embodiment of the compounds of formula IG, L³ is —C(O)—, L⁴ is —CH(CH₃)—, and Y is substituted or unsubstituted indolyl.

In another embodiment of the compounds of formula IG, L³ is —S(O)₂—, L⁴ is —CH(CH₃)—, and Y is substituted or unsubstituted indolyl.

In another embodiment of the compounds of formula IG, L³ is —C(R⁴)₂—, L⁴ is —C(O)—, and Y is substituted or unsubstituted indolyl.

In another embodiment of the compounds of formula IG, L³ is —CH₂—, L⁴ is —C(O)—, and Y is substituted or unsubstituted indolyl.

In another embodiment of the compounds of formula IG, L³ is —CH(CH₃)—, L⁴ is —C(O)—, and Y is substituted or unsubstituted indolyl.

In another embodiment of the compounds of formula IG, L³ is —C(O)—, L⁴ is —C(O)—, and Y is substituted or unsubstituted indolyl.

In another embodiment of the compounds of formula IG, L³ is —S(O)₂—, L⁴ is —C(O)—, and Y is substituted or unsubstituted indolyl.

In another embodiment of the compounds of formula IG, L³ is —C(R⁴)₂—, L⁴ is —S(O)₂—, and Y is substituted or unsubstituted indolyl.

In another embodiment of the compounds of formula IG, L³ is —CH₂—, L⁴ is —S(O)₂—, and Y is substituted or unsubstituted indolyl.

In another embodiment of the compounds of formula IG, L³ is —CH(CH₃)—, L⁴ is —S(O)₂—, and Y is substituted or unsubstituted indolyl.

In another embodiment of the compounds of formula IG, L³ is —C(O)—, L⁴ is —S(O)₂—, and Y is substituted or unsubstituted indolyl.

In another embodiment of the compounds of formula IG, L³ is —S(O)₂—, L⁴ is —S(O)₂—, and Y is substituted or unsubstituted indolyl.

In another embodiment of the compounds of formula IG, L³ is —C(R⁴)₂—, L⁴ is a covalent bond, and Y is substituted or unsubstituted benzimidazolyl.

In another embodiment of the compounds of formula IG, L³ is —CH₂—, L⁴ is a covalent bond, and Y is substituted or unsubstituted benzimidazolyl.

In another embodiment of the compounds of formula IG, L³ is —CH(CH₃)—, L⁴ is a covalent bond, and Y is substituted or unsubstituted benzimidazolyl.

In another embodiment of the compounds of formula IG, L³ is —C(O)—, L⁴ is a covalent bond, and Y is substituted or unsubstituted benzimidazolyl.

In another embodiment of the compounds of formula IG, L³ is —S(O)₂—, L⁴ is a covalent bond, and Y is substituted or unsubstituted benzimidazolyl.

In another embodiment of the compounds of formula IG, L³ is —C(R⁴)₂—, L⁴ is —C(R⁴)₂—, and Y is substituted or unsubstituted benzimidazolyl.

In another embodiment of the compounds of formula IG, L³ is —CH₂—, L⁴ is —C(R⁴)₂—, and Y is substituted or unsubstituted benzimidazolyl.

In another embodiment of the compounds of formula IG, L³ is —CH(CH₃)—, L⁴ is —C(R⁴)₂—, and Y is substituted or unsubstituted benzimidazolyl.

In another embodiment of the compounds of formula IG, L³ is —C(O)—, L⁴ is —C(R⁴)₂—, and Y is substituted or unsubstituted benzimidazolyl.

In another embodiment of the compounds of formula IG, L³ is —S(O)₂—, L⁴ is —C(R⁴)₂—, and Y is substituted or unsubstituted benzimidazolyl.

In another embodiment of the compounds of formula IG, L³ is —C(R⁴)₂—, L⁴ is —CH₂—, and Y is substituted or unsubstituted benzimidazolyl.

In another embodiment of the compounds of formula IG, L³ is —CH₂—, L⁴ is —CH₂—, and Y is substituted or unsubstituted benzimidazolyl.

In another embodiment of the compounds of formula IG, L³ is —CH(CH₃)—, L⁴ is —CH₂—, and Y is substituted or unsubstituted benzimidazolyl.

In another embodiment of the compounds of formula IG, L³ is —C(O)—, L⁴ is —CH₂—, and Y is substituted or unsubstituted benzimidazolyl.

In another embodiment of the compounds of formula IG, L³ is —S(O)₂—, L⁴ is —CH₂—, and Y is substituted or unsubstituted benzimidazolyl.

In another embodiment of the compounds of formula IG, L³ is —C(R⁴)₂—, L⁴ is —CH(CH₃)—, and Y is substituted or unsubstituted benzimidazolyl.

In another embodiment of the compounds of formula IG, L³ is —CH₂—, L⁴ is —CH(CH₃)—, and Y is substituted or unsubstituted benzimidazolyl.

In another embodiment of the compounds of formula IG, L³ is —CH(CH₃)—, L⁴ is —CH(CH₃)—, and Y is substituted or unsubstituted benzimidazolyl.

In another embodiment of the compounds of formula IG, L³ is —C(O)—, L⁴ is —CH(CH₃)—, and Y is substituted or unsubstituted benzimidazolyl.

In another embodiment of the compounds of formula IG, L³ is —S(O)₂—, L⁴ is —CH(CH₃)—, and Y is substituted or unsubstituted benzimidazolyl.

In another embodiment of the compounds of formula IG, L³ is —C(R⁴)₂—, L⁴ is —C(O)—, and Y is substituted or unsubstituted benzimidazolyl.

In another embodiment of the compounds of formula IG, L³ is —CH₂—, L⁴ is —C(O)—, and Y is substituted or unsubstituted benzimidazolyl.

In another embodiment of the compounds of formula IG, L³ is —CH(CH₃)—, L⁴ is —C(O)—, and Y is substituted or unsubstituted benzimidazolyl.

In another embodiment of the compounds of formula IG, L³ is —C(O)—, L⁴ is —C(O)—, and Y is substituted or unsubstituted benzimidazolyl.

In another embodiment of the compounds of formula IG, L³ is —S(O)₂—, L⁴ is —C(O)—, and Y is substituted or unsubstituted benzimidazolyl.

In another embodiment of the compounds of formula IG, L³ is —C(R⁴)₂—, L⁴ is —S(O)₂—, and Y is substituted or unsubstituted benzimidazolyl.

In another embodiment of the compounds of formula IG, L³ is —CH₂—, L⁴ is —S(O)₂—, and Y is substituted or unsubstituted benzimidazolyl.

In another embodiment of the compounds of formula IG, L³ is —CH(CH₃)—, L⁴ is —S(O)₂—, and Y is substituted or unsubstituted benzimidazolyl.

In another embodiment of the compounds of formula IG, L³ is —C(O)—, L⁴ is —S(O)₂—, and Y is substituted or unsubstituted benzimidazolyl.

In another embodiment of the compounds of formula IG, L³ is —S(O)₂—, L⁴ is —S(O)₂—, and Y is substituted or unsubstituted benzimidazolyl.

In another embodiment of the compounds of formula IG, L³ is —C(R⁴)₂—, L⁴ is a covalent bond, and Y is substituted or unsubstituted purinyl.

In another embodiment of the compounds of formula IG, L³ is —CH₂—, L⁴ is a covalent bond, and Y is substituted or unsubstituted purinyl.

In another embodiment of the compounds of formula IG, L³ is —CH(CH₃)—, L⁴ is a covalent bond, and Y is substituted or unsubstituted purinyl.

In another embodiment of the compounds of formula IG, L³ is —C(O)—, L⁴ is a covalent bond, and Y is substituted or unsubstituted purinyl.

In another embodiment of the compounds of formula IG, L³ is —S(O)₂—, L⁴ is a covalent bond, and Y is substituted or unsubstituted purinyl.

In another embodiment of the compounds of formula IG, L³ is —C(R⁴)₂—, L⁴ is —C(R⁴)₂—, and Y is substituted or unsubstituted purinyl.

In another embodiment of the compounds of formula IG, L³ is —CH₂—, L⁴ is —C(R⁴)₂—, and Y is substituted or unsubstituted purinyl.

In another embodiment of the compounds of formula IG, L³ is —CH(CH₃)—, L⁴ is —C(R⁴)₂—, and Y is substituted or unsubstituted purinyl.

In another embodiment of the compounds of formula IG, L³ is —C(O)—, L⁴ is —C(R⁴)₂—, and Y is substituted or unsubstituted purinyl.

In another embodiment of the compounds of formula IG, L³ is —S(O)₂—, L⁴ is —C(R⁴)₂—, and Y is substituted or unsubstituted purinyl.

In another embodiment of the compounds of formula IG, L³ is —C(R⁴)₂—, L⁴ is —CH₂—, and Y is substituted or unsubstituted purinyl.

In another embodiment of the compounds of formula IG, L³ is —CH₂—, L⁴ is —CH₂—, and Y is substituted or unsubstituted purinyl.

In another embodiment of the compounds of formula IG, L³ is —CH(CH₃)—, L⁴ is —CH₂—, and Y is substituted or unsubstituted purinyl.

In another embodiment of the compounds of formula IG, L³ is —C(O)—, L⁴ is —CH₂—, and Y is substituted or unsubstituted purinyl.

In another embodiment of the compounds of formula IG, L³ is —S(O)₂—, L⁴ is —CH₂—, and Y is substituted or unsubstituted purinyl.

In another embodiment of the compounds of formula IG, L³ is —C(R⁴)₂—, L⁴ is —CH(CH₃)—, and Y is substituted or unsubstituted purinyl.

In another embodiment of the compounds of formula IG, L³ is —CH₂—, L⁴ is —CH(CH₃)—, and Y is substituted or unsubstituted purinyl.

In another embodiment of the compounds of formula IG, L³ is —CH(CH₃)—, L⁴ is —CH(CH₃)—, and Y is substituted or unsubstituted purinyl.

In another embodiment of the compounds of formula IG, L³ is —C(O)—, L⁴ is —CH(CH₃)—, and Y is substituted or unsubstituted purinyl.

In another embodiment of the compounds of formula IG, L³ is —S(O)₂—, L⁴ is —CH(CH₃)—, and Y is substituted or unsubstituted purinyl.

In another embodiment of the compounds of formula IG, L³ is —C(R⁴)₂—, L⁴ is —C(O)—, and Y is substituted or unsubstituted purinyl.

In another embodiment of the compounds of formula IG, L³ is —CH₂—, L⁴ is —C(O)—, and Y is substituted or unsubstituted purinyl.

In another embodiment of the compounds of formula IG, L³ is —CH(CH₃)—, L⁴ is —C(O)—, and Y is substituted or unsubstituted purinyl.

In another embodiment of the compounds of formula IG, L³ is —C(O)—, L⁴ is —C(O)—, and Y is substituted or unsubstituted purinyl.

In another embodiment of the compounds of formula IG, L³ is —S(O)₂—, L⁴ is —C(O)—, and Y is substituted or unsubstituted purinyl.

In another embodiment of the compounds of formula IG, L³ is —C(R⁴)₂—, L⁴ is —S(O)₂—, and Y is substituted or unsubstituted purinyl.

In another embodiment of the compounds of formula IG, L³ is —CH₂—, L⁴ is —S(O)₂—, and Y is substituted or unsubstituted purinyl.

In another embodiment of the compounds of formula IG, L³ is —CH(CH₃)—, L⁴ is —S(O)₂—, and Y is substituted or unsubstituted purinyl.

In another embodiment of the compounds of formula IG, L³ is —C(O)—, L⁴ is —S(O)₂—, and Y is substituted or unsubstituted purinyl.

In another embodiment of the compounds of formula IG, L³ is —S(O)₂—, L⁴ is —S(O)₂—, and Y is substituted or unsubstituted purinyl.

In another embodiment of the compounds of formula IG, L³ is —C(R⁴)₂—, L⁴ is a covalent bond, and Y is substituted or unsubstituted thienyl.

In another embodiment of the compounds of formula IG, L³ is —CH₂—, L⁴ is a covalent bond, and Y is substituted or unsubstituted thienyl.

In another embodiment of the compounds of formula IG, L³ is —CH(CH₃)—, L⁴ is a covalent bond, and Y is substituted or unsubstituted thienyl.

In another embodiment of the compounds of formula IG, L³ is —C(O)—, L⁴ is a covalent bond, and Y is substituted or unsubstituted thienyl.

In another embodiment of the compounds of formula IG, L³ is —S(O)₂—, L⁴ is a covalent bond, and Y is substituted or unsubstituted thienyl.

In another embodiment of the compounds of formula IG, L³ is —C(R⁴)₂—, L⁴ is —C(R⁴)₂—, and Y is substituted or unsubstituted thienyl.

In another embodiment of the compounds of formula IG, L³ is —CH₂—, L⁴ is —C(R⁴)₂—, and Y is substituted or unsubstituted thienyl.

In another embodiment of the compounds of formula IG, L³ is —CH(CH₃)—, L⁴ is —C(R⁴)₂—, and Y is substituted or unsubstituted thienyl.

In another embodiment of the compounds of formula IG, L³ is —C(O)—, L⁴ is —C(R⁴)₂—, and Y is substituted or unsubstituted thienyl.

In another embodiment of the compounds of formula IG, L³ is —S(O)₂—, L⁴ is —C(R⁴)₂—, and Y is substituted or unsubstituted thienyl.

In another embodiment of the compounds of formula IG, L³ is —C(R⁴)₂—, L⁴ is —CH₂—, and Y is substituted or unsubstituted thienyl.

In another embodiment of the compounds of formula IG, L³ is —CH₂—, L⁴ is —CH₂—, and Y is substituted or unsubstituted thienyl.

In another embodiment of the compounds of formula IG, L³ is —CH(CH₃)—, L⁴ is —CH₂—, and Y is substituted or unsubstituted thienyl.

In another embodiment of the compounds of formula IG, L³ is —C(O)—, L⁴ is —CH₂—, and Y is substituted or unsubstituted thienyl.

In another embodiment of the compounds of formula IG, L³ is —S(O)₂—, L⁴ is —CH₂—, and Y is substituted or unsubstituted thienyl.

In another embodiment of the compounds of formula IG, L³ is —C(R⁴)₂—, L⁴ is —CH(CH₃)—, and Y is substituted or unsubstituted thienyl.

In another embodiment of the compounds of formula IG, L³ is —CH₂—, L⁴ is —CH(CH₃)—, and Y is substituted or unsubstituted thienyl.

In another embodiment of the compounds of formula IG, L³ is —CH(CH₃)—, L⁴ is —CH(CH₃)—, and Y is substituted or unsubstituted thienyl.

In another embodiment of the compounds of formula IG, L³ is —C(O)—, L⁴ is —CH(CH₃)—, and Y is substituted or unsubstituted thienyl.

In another embodiment of the compounds of formula IG, L³ is —S(O)₂—, L⁴ is —CH(CH₃)—, and Y is substituted or unsubstituted thienyl.

In another embodiment of the compounds of formula IG, L³ is —C(R⁴)₂—, L⁴ is —C(O)—, and Y is substituted or unsubstituted thienyl.

In another embodiment of the compounds of formula IG, L³ is —CH₂—, L⁴ is —C(O)—, and Y is substituted or unsubstituted thienyl.

In another embodiment of the compounds of formula IG, L³ is —CH(CH₃)—, L⁴ is —C(O)—, and Y is substituted or unsubstituted thienyl.

In another embodiment of the compounds of formula IG, L³ is —C(O)—, L⁴ is —C(O)—, and Y is substituted or unsubstituted thienyl.

In another embodiment of the compounds of formula IG, L³ is —S(O)₂—, L⁴ is —C(O)—, and Y is substituted or unsubstituted thienyl.

In another embodiment of the compounds of formula IG, L³ is —C(R⁴)₂—, L⁴ is —S(O)₂—, and Y is substituted or unsubstituted thienyl.

In another embodiment of the compounds of formula IG, L³ is —CH₂—, L⁴ is —S(O)₂—, and Y is substituted or unsubstituted thienyl.

In another embodiment of the compounds of formula IG, L³ is —CH(CH₃)—, L⁴ is —S(O)₂—, and Y is substituted or unsubstituted thienyl.

In another embodiment of the compounds of formula IG, L³ is —C(O)—, L⁴ is —S(O)₂—, and Y is substituted or unsubstituted thienyl.

In another embodiment of the compounds of formula IG, L³ is —S(O)₂—, L⁴ is —S(O)₂—, and Y is substituted or unsubstituted thienyl.

In another embodiment of the compounds of formula IG, L³ is —C(R⁴)₂—, L⁴ is a covalent bond, and Y is substituted or unsubstituted furanyl.

In another embodiment of the compounds of formula IG, L³ is —CH₂—, L⁴ is a covalent bond, and Y is substituted or unsubstituted furanyl.

In another embodiment of the compounds of formula IG, L³ is —CH(CH₃)—, L⁴ is a covalent bond, and Y is substituted or unsubstituted furanyl.

In another embodiment of the compounds of formula IG, L³ is —C(O)—, L⁴ is a covalent bond, and Y is substituted or unsubstituted furanyl.

In another embodiment of the compounds of formula IG, L³ is —S(O)₂—, L⁴ is a covalent bond, and Y is substituted or unsubstituted furanyl.

In another embodiment of the compounds of formula IG, L³ is —C(R⁴)₂—, L⁴ is —C(R⁴)₂—, and Y is substituted or unsubstituted furanyl.

In another embodiment of the compounds of formula IG, L³ is —CH₂—, L⁴ is —C(R⁴)₂—, and Y is substituted or unsubstituted furanyl.

In another embodiment of the compounds of formula IG, L³ is —CH(CH₃)—, L⁴ is —C(R⁴)₂—, and Y is substituted or unsubstituted furanyl.

In another embodiment of the compounds of formula IG, L³ is —C(O)—, L⁴ is —C(R⁴)₂—, and Y is substituted or unsubstituted furanyl.

In another embodiment of the compounds of formula IG, L³ is —S(O)₂—, L⁴ is —C(R⁴)₂—, and Y is substituted or unsubstituted furanyl.

In another embodiment of the compounds of formula IG, L³ is —C(R⁴)₂—, L⁴ is —CH₂—, and Y is substituted or unsubstituted furanyl.

In another embodiment of the compounds of formula IG, L³ is —CH₂—, L⁴ is —CH₂—, and Y is substituted or unsubstituted furanyl.

In another embodiment of the compounds of formula IG, L³ is —CH(CH₃)—, L⁴ is —CH₂—, and Y is substituted or unsubstituted furanyl.

In another embodiment of the compounds of formula IG, L³ is —C(O)—, L⁴ is —CH₂—, and Y is substituted or unsubstituted furanyl.

In another embodiment of the compounds of formula IG, L³ is —S(O)₂—, L⁴ is —CH₂—, and Y is substituted or unsubstituted furanyl.

In another embodiment of the compounds of formula IG, L³ is —C(R⁴)₂—, L⁴ is —CH(CH₃)—, and Y is substituted or unsubstituted furanyl.

In another embodiment of the compounds of formula IG, L³ is —CH₂—, L⁴ is —CH(CH₃)—, and Y is substituted or unsubstituted furanyl.

In another embodiment of the compounds of formula IG, L³ is —CH(CH₃)—, L⁴ is —CH(CH₃)—, and Y is substituted or unsubstituted furanyl.

In another embodiment of the compounds of formula IG, L³ is —C(O)—, L⁴ is —CH(CH₃)—, and Y is substituted or unsubstituted furanyl.

In another embodiment of the compounds of formula IG, L³ is —S(O)₂—, L⁴ is —CH(CH₃)—, and Y is substituted or unsubstituted furanyl.

In another embodiment of the compounds of formula IG, L³ is —C(R⁴)₂—, L⁴ is —C(O)—, and Y is substituted or unsubstituted furanyl.

In another embodiment of the compounds of formula IG, L³ is —CH₂—, L⁴ is —C(O)—, and Y is substituted or unsubstituted furanyl.

In another embodiment of the compounds of formula IG, L³ is —CH(CH₃)—, L⁴ is —C(O)—, and Y is substituted or unsubstituted furanyl.

In another embodiment of the compounds of formula IG, L³ is —C(O)—, L⁴ is —C(O)—, and Y is substituted or unsubstituted furanyl.

In another embodiment of the compounds of formula IG, L³ is —S(O)₂—, L⁴ is —C(O)—, and Y is substituted or unsubstituted furanyl.

In another embodiment of the compounds of formula IG, L³ is —C(R⁴)₂—, L⁴ is —S(O)₂—, and Y is substituted or unsubstituted furanyl.

In another embodiment of the compounds of formula IG, L³ is —CH₂—, L⁴ is —S(O)₂—, and Y is substituted or unsubstituted furanyl.

In another embodiment of the compounds of formula IG, L³ is —CH(CH₃)—, L⁴ is —S(O)₂—, and Y is substituted or unsubstituted furanyl.

In another embodiment of the compounds of formula IG, L³ is —C(O)—, L⁴ is —S(O)₂—, and Y is substituted or unsubstituted furanyl.

In another embodiment of the compounds of formula IG, L³ is —S(O)₂—, L⁴ is —S(O)₂—, and Y is substituted or unsubstituted furanyl.

In another embodiment of the compounds of formula IG, L³ is —C(R⁴)₂—, L⁴ is a covalent bond, and Y is substituted or unsubstituted pyrrolyl.

In another embodiment of the compounds of formula IG, L³ is —CH₂—, L⁴ is a covalent bond, and Y is substituted or unsubstituted pyrrolyl.

In another embodiment of the compounds of formula IG, L³ is —CH(CH₃)—, L⁴ is a covalent bond, and Y is substituted or unsubstituted pyrrolyl.

In another embodiment of the compounds of formula IG, L³ is —C(O)—, L⁴ is a covalent bond, and Y is substituted or unsubstituted pyrrolyl.

In another embodiment of the compounds of formula IG, L³ is —S(O)₂—, L⁴ is a covalent bond, and Y is substituted or unsubstituted pyrrolyl.

In another embodiment of the compounds of formula IG, L³ is —C(R⁴)₂—, L⁴ is —C(R⁴)₂—, and Y is substituted or unsubstituted pyrrolyl.

In another embodiment of the compounds of formula IG, L³ is —CH₂—, L⁴ is —C(R⁴)₂—, and Y is substituted or unsubstituted pyrrolyl.

In another embodiment of the compounds of formula IG, L³ is —CH(CH₃)—, L⁴ is —C(R⁴)₂—, and Y is substituted or unsubstituted pyrrolyl.

In another embodiment of the compounds of formula IG, L³ is —C(O)—, L⁴ is —C(R⁴)₂—, and Y is substituted or unsubstituted pyrrolyl.

In another embodiment of the compounds of formula IG, L³ is —S(O)₂—, L⁴ is —C(R⁴)₂—, and Y is substituted or unsubstituted pyrrolyl.

In another embodiment of the compounds of formula IG, L³ is —C(R⁴)₂—, L⁴ is —CH₂—, and Y is substituted or unsubstituted pyrrolyl.

In another embodiment of the compounds of formula IG, L³ is —CH₂—, L⁴ is —CH₂—, and Y is substituted or unsubstituted pyrrolyl.

In another embodiment of the compounds of formula IG, L³ is —CH(CH₃)—, L⁴ is —CH₂—, and Y is substituted or unsubstituted pyrrolyl.

In another embodiment of the compounds of formula IG, L³ is —C(O)—, L⁴ is —CH₂—, and Y is substituted or unsubstituted pyrrolyl.

In another embodiment of the compounds of formula IG, L³ is —S(O)₂—, L⁴ is —CH₂—, and Y is substituted or unsubstituted pyrrolyl.

In another embodiment of the compounds of formula IG, L³ is —C(R⁴)₂—, L⁴ is —CH(CH₃)—, and Y is substituted or unsubstituted pyrrolyl.

In another embodiment of the compounds of formula IG, L³ is —CH₂—, L⁴ is —CH(CH₃)—, and Y is substituted or unsubstituted pyrrolyl.

In another embodiment of the compounds of formula IG, L³ is —CH(CH₃)—, L⁴ is —CH(CH₃)—, and Y is substituted or unsubstituted pyrrolyl.

In another embodiment of the compounds of formula IG, L³ is —C(O)—, L⁴ is —CH(CH₃)—, and Y is substituted or unsubstituted pyrrolyl.

In another embodiment of the compounds of formula IG, L³ is —S(O)₂—, L⁴ is —CH(CH₃)—, and Y is substituted or unsubstituted pyrrolyl.

In another embodiment of the compounds of formula IG, L³ is —C(R⁴)₂—, L⁴ is —C(O)—, and Y is substituted or unsubstituted pyrrolyl.

In another embodiment of the compounds of formula IG, L³ is —CH₂—, L⁴ is —C(O)—, and Y is substituted or unsubstituted pyrrolyl.

In another embodiment of the compounds of formula IG, L³ is —CH(CH₃)—, L⁴ is —C(O)—, and Y is substituted or unsubstituted pyrrolyl.

In another embodiment of the compounds of formula IG, L³ is —C(O)—, L⁴ is —C(O)—, and Y is substituted or unsubstituted pyrrolyl.

In another embodiment of the compounds of formula IG, L³ is —S(O)₂—, L⁴ is —C(O)—, and Y is substituted or unsubstituted pyrrolyl.

In another embodiment of the compounds of formula IG, L³ is —C(R⁴)₂—, L⁴ is —S(O)₂—, and Y is substituted or unsubstituted pyrrolyl.

In another embodiment of the compounds of formula IG, L³ is —CH₂—, L⁴ is —S(O)₂—, and Y is substituted or unsubstituted pyrrolyl.

In another embodiment of the compounds of formula IG, L³ is —CH(CH₃)—, L⁴ is —S(O)₂—, and Y is substituted or unsubstituted pyrrolyl.

In another embodiment of the compounds of formula IG, L³ is —C(O)—, L⁴ is —S(O)₂—, and Y is substituted or unsubstituted pyrrolyl.

In another embodiment of the compounds of formula IG, L³ is —S(O)₂—, L⁴ is —S(O)₂—, and Y is substituted or unsubstituted pyrrolyl.

In another embodiment, the compounds of the present invention, or pharmaceutically acceptable salts, solvates, prodrugs, tautomers, or esters thereof, have the structure of formula (IG-1):

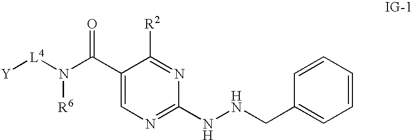

wherein R¹, R⁵, L², L⁵, n, X and Z are as defined herein.

In another embodiment of the compounds of formula IG-1, L⁴ is selected from the group consisting of a covalent bond, —CH₂—, and —CH(aminopropyl); Y is selected from the group consisting of substituted or unsubstituted phenyl, substituted or unsubstituted 5,6,7,8-tetrahydroquinolin-8-yl, and substituted or unsubstituted benzimidazolyl; R² is selected from the group consisting of H, methyl, ethyl, and trifluoromethyl; and R⁶ is selected from the group consisting of H, methyl, and aminobutyl.

In another embodiment of the compounds of formula IG-1, R⁶ is H.

In another embodiment of the compounds of formula IG-1, R⁶ is alkyl.

In another embodiment of the compounds of formula IG-1, R⁶ is methyl.

In another embodiment of the compounds of formula IG-1, R⁶ is substituted or unsubstituted benzyl.

In another embodiment of the compounds of formula IG-1, R⁶ is aminoalkyl. In another embodiment of the compounds of formula IG-1, L⁴ is a covalent bond.

In another embodiment of the compounds of formula IG-1, L⁴ is —C(R⁴)₂—.

In another embodiment of the compounds of formula IG-1, L⁴ is —CH₂—.

In another embodiment of the compounds of formula IG-1, L⁴ is —CH(CH₃)—.

In another embodiment of the compounds of formula IG-1, L⁴ is —CH(aminoalkyl)-.

In another embodiment of the compounds of formula IG-1, L⁴ is —CH(NH₂alkyl)-.

In another embodiment of the compounds of formula IG-1, L⁴ is —CH(NH(alkyl)alkyl)-.

In another embodiment of the compounds of formula IG-1, L⁴ is —CH(N(alkyl)₂alkyl)-. In another embodiment of the compounds of formula IG-1, L⁴ is —CH(NH₂(CH₂)₃)—. In another embodiment of the compounds of formula IG-1, Y is substituted or unsubstituted aryl.

In another embodiment of the compounds of formula IG-1, Y is substituted or unsubstituted phenyl.

In another embodiment of the compounds of formula IG-1, Y is substituted or unsubstituted heteroaryl.

In another embodiment of the compounds of formula IG-1, Y is substituted or unsubstituted pyridyl.

In another embodiment of the compounds of formula IG-1, Y is substituted or unsubstituted pyrimidyl.

In another embodiment of the compounds of formula IG-1, Y is substituted or unsubstituted indolyl.

In another embodiment of the compounds of formula IG-1, Y is substituted or unsubstituted benzimidazolyl.

In another embodiment of the compounds of formula IG-1, Y is substituted or unsubstituted purinyl.

In another embodiment of the compounds of formula IG-1, Y is substituted or unsubstituted thienyl.

In another embodiment of the compounds of formula IG-1, Y is substituted or unsubstituted quinolyl.

In another embodiment of the compounds of formula IG-1, Y is substituted or unsubstituted furanyl.

In another embodiment of the compounds of formula IG-1, Y is substituted or unsubstituted pyrrolyl.

In another embodiment of the compounds of formula IG-1, Y is substituted or unsubstituted 5,6,7,8,-tetrahydroquinolin-8-yl.

In another embodiment of the compounds of formula IG-1, L⁴ is a covalent bond, and Y is substituted or unsubstituted cycloalkyl.

In another embodiment of the compounds of formula IG-1, L⁴ is a covalent bond, and Y is substituted or unsubstituted 5,6,7,8,-tetrahydroquinolin-8-yl.

In another embodiment of the compounds of formula IG-1, L⁴ is —C(R⁴)₂—, and Y is substituted or unsubstituted aryl.

In another embodiment of the compounds of formula IG-1, L⁴ is —CH₂—, and Y is substituted or unsubstituted aryl.

In another embodiment of the compounds of formula IG-1, L⁴ is —CH(CH₃)—, and Y is substituted or unsubstituted aryl.

In another embodiment of the compounds of formula IG-1, L⁴ is —CH(aminoalkyl)-, and Y is substituted or unsubstituted aryl.

In another embodiment of the compounds of formula IG-1, L⁴ is —CH(NH₂alkyl)-, and Y is substituted or unsubstituted aryl.

In another embodiment of the compounds of formula IG-1, L⁴ is —CH(NH(alkyl)alkyl)-, and Y is substituted or unsubstituted aryl.

In another embodiment of the compounds of formula IG-1, L⁴ is —CH(N(alkyl)₂alkyl)-, and Y is substituted or unsubstituted aryl.

In another embodiment of the compounds of formula IG-1, L⁴ is —CH(NH₂(CH₂)₃)—, and Y is substituted or unsubstituted aryl.

In another embodiment of the compounds of formula IG-1, L⁴ is —C(R⁴)₂—, and Y is substituted or unsubstituted phenyl.

In another embodiment of the compounds of formula IG-1, L⁴ is —CH₂—, and Y is substituted or unsubstituted phenyl. In another embodiment of the compounds of formula IG-1, L⁴ is —CH(CH₃)—, and Y is substituted or unsubstituted phenyl.

In another embodiment of the compounds of formula IG-1, L⁴ is —CH(aminoalkyl)-, and Y is substituted or unsubstituted phenyl.

In another embodiment of the compounds of formula IG-1, L⁴ is —CH(NH₂alkyl)-, and Y is substituted or unsubstituted phenyl.

In another embodiment of the compounds of formula IG-1, L⁴ is —CH(NH(alkyl)alkyl)-, and Y is substituted or unsubstituted phenyl.

In another embodiment of the compounds of formula IG-1, L⁴ is —CH(N(alkyl)₂alkyl)-, and Y is substituted or unsubstituted phenyl.

In another embodiment of the compounds of formula IG-1, L⁴ is —CH(NH₂(CH₂)₃)—, and Y is substituted or unsubstituted phenyl.

In another embodiment of the compounds of formula IG-1, L⁴ is —C(R⁴)₂—, and Y is substituted or unsubstituted heteroaryl.

In another embodiment of the compounds of formula IG-1, L⁴ is —CH₂—, and Y is substituted or unsubstituted heteroaryl.

In another embodiment of the compounds of formula IG-1, L⁴ is —CH(CH₃)—, and Y is substituted or unsubstituted heteroaryl.

In another embodiment of the compounds of formula IG-1, L⁴ is —CH(aminoalkyl)-, and Y is substituted or unsubstituted heteroaryl.

In another embodiment of the compounds of formula IG-1, L⁴ is —CH(NH₂alkyl)-, and Y is substituted or unsubstituted heteroaryl.

In another embodiment of the compounds of formula IG-1, L⁴ is —CH(NH(alkyl)alkyl)-, and Y is substituted or unsubstituted heteroaryl.

In another embodiment of the compounds of formula IG-1, L⁴ is —CH(N(alkyl)₂alkyl)-, and Y is substituted or unsubstituted heteroaryl.

In another embodiment of the compounds of formula IG-1, L⁴ is —CH(NH₂(CH₂)₃)—, and Y is substituted or unsubstituted heteroaryl.

In another embodiment of the compounds of formula IG-1, L⁴ is —C(R⁴)₂—, and Y is substituted or unsubstituted pyridyl.

In another embodiment of the compounds of formula IG-1, L⁴ is —CH₂—, and Y is substituted or unsubstituted pyridyl.

In another embodiment of the compounds of formula IG-1, L⁴ is —CH(CH₃)—, and Y is substituted or unsubstituted pyridyl.

In another embodiment of the compounds of formula IG-1, L⁴ is —CH(aminoalkyl)-, and Y is substituted or unsubstituted pyridyl.

In another embodiment of the compounds of formula IG-1, L⁴ is —CH(NH₂alkyl)-, and Y is substituted or unsubstituted pyridyl.

In another embodiment of the compounds of formula IG-1, L⁴ is —CH(NH(alkyl)alkyl)-, and Y is substituted or unsubstituted pyridyl.

In another embodiment of the compounds of formula IG-1, L⁴ is —CH(N(alkyl)₂alkyl)-, and Y is substituted or unsubstituted pyridyl.

In another embodiment of the compounds of formula IG-1, L⁴ is —CH(NH₂(CH₂)₃)—, and Y is substituted or unsubstituted pyridyl.

In another embodiment of the compounds of formula IG-1, L⁴ is —C(R⁴)₂—, and Y is substituted or unsubstituted pyrimidyl.

In another embodiment of the compounds of formula IG-1, L⁴ is —CH₂—, and Y is substituted or unsubstituted pyrimidyl.

In another embodiment of the compounds of formula IG-1, L⁴ is —CH(CH₃)—, and Y is substituted or unsubstituted pyrimidyl.

In another embodiment of the compounds of formula IG-1, L⁴ is —CH(aminoalkyl)-, and Y is substituted or unsubstituted pyrimidyl.

In another embodiment of the compounds of formula IG-1, L⁴ is —CH(NH₂alkyl)-, and Y is substituted or unsubstituted pyrimidyl.

In another embodiment of the compounds of formula IG-1, L⁴ is —CH(NH(alkyl)alkyl)-, and Y is substituted or unsubstituted pyrimidyl.

In another embodiment of the compounds of formula IG-1, L⁴ is —CH(N(alkyl)₂alkyl)-, and Y is substituted or unsubstituted pyrimidyl.

In another embodiment of the compounds of formula IG-1, L⁴ is —CH(NH₂(CH₂)₃)—, and Y is substituted or unsubstituted pyrimidyl.

In another embodiment of the compounds of formula IG-1, L⁴ is —C(R⁴)₂—, and Y is substituted or unsubstituted quinolyl.

In another embodiment of the compounds of formula IG-1, L⁴ is —CH₂—, and Y is substituted or unsubstituted quinolyl.

In another embodiment of the compounds of formula IG-1, L⁴ is —CH(CH₃)—, and Y is substituted or unsubstituted quinolyl.

In another embodiment of the compounds of formula IG-1, L⁴ is —CH(aminoalkyl)-, and Y is substituted or unsubstituted quinolyl.

In another embodiment of the compounds of formula IG-1, L⁴ is —CH(NH₂alkyl)-, and Y is substituted or unsubstituted quinolyl.

In another embodiment of the compounds of formula IG-1, L⁴ is —CH(NH(alkyl)alkyl)-, and Y is substituted or unsubstituted quinolyl.

In another embodiment of the compounds of formula IG-1, L⁴ is —CH(N(alkyl)₂alkyl)-, and Y is substituted or unsubstituted quinolyl.

In another embodiment of the compounds of formula IG-1, L⁴ is —CH(NH₂(CH₂)₃)—, and Y is substituted or unsubstituted quinolyl.

In another embodiment of the compounds of formula IG-1, L⁴ is —C(R⁴)₂—, and Y is substituted or unsubstituted indolyl.

In another embodiment of the compounds of formula IG-1, L⁴ is —CH₂—, and Y is substituted or unsubstituted indolyl.

In another embodiment of the compounds of formula IG-1, L⁴ is —CH(CH₃)—, and Y is substituted or unsubstituted indolyl.

In another embodiment of the compounds of formula IG-1, L⁴ is —CH(aminoalkyl)-, and Y is substituted or unsubstituted indolyl.

In another embodiment of the compounds of formula IG-1, L⁴ is —CH(NH₂alkyl)-, and Y is substituted or unsubstituted indolyl.

In another embodiment of the compounds of formula IG-1, L⁴ is —CH(NH(alkyl)alkyl)-, and Y is substituted or unsubstituted indolyl.

In another embodiment of the compounds of formula IG-1, L⁴ is —CH(N(alkyl)₂alkyl)-, and Y is substituted or unsubstituted indolyl.

In another embodiment of the compounds of formula IG-1, L⁴ is —CH(NH₂(CH₂)₃)—, and Y is substituted or unsubstituted indolyl.

In another embodiment of the compounds of formula IG-1, L⁴ is —C(R⁴)₂—, and Y is substituted or unsubstituted benzimidazolyl.

In another embodiment of the compounds of formula IG-1, L⁴ is —CH₂—, and Y is substituted or unsubstituted benzimidazolyl.

In another embodiment of the compounds of formula IG-1, L⁴ is —CH(CH₃)—, and Y is substituted or unsubstituted benzimidazolyl.

In another embodiment of the compounds of formula IG-1, L⁴ is —CH(aminoalkyl)-, and Y is substituted or unsubstituted benzimidazolyl.

In another embodiment of the compounds of formula IG-1, L⁴ is —CH(NH₂alkyl)-, and Y is substituted or unsubstituted benzimidazolyl.

In another embodiment of the compounds of formula IG-1, L⁴ is —CH(NH(alkyl)alkyl)-, and Y is substituted or unsubstituted benzimidazolyl.

In another embodiment of the compounds of formula IG-1, L⁴ is —CH(N(alkyl)₂alkyl)-, and Y is substituted or unsubstituted benzimidazolyl.

In another embodiment of the compounds of formula IG-1, L⁴ is —CH(NH₂(CH₂)₃)—, and Y is substituted or unsubstituted benzimidazolyl.

In another embodiment of the compounds of formula IG-1, L⁴ is —C(R⁴)₂—, and Y is substituted or unsubstituted purinyl.

In another embodiment of the compounds of formula IG-1, L⁴ is —CH₂—, and Y is substituted or unsubstituted purinyl.

In another embodiment of the compounds of formula IG-1, L⁴ is —CH(CH₃)—, and Y is substituted or unsubstituted purinyl.

In another embodiment of the compounds of formula IG-1, L⁴ is —CH(aminoalkyl)-, and Y is substituted or unsubstituted purinyl.

In another embodiment of the compounds of formula IG-1, L⁴ is —CH(NH₂alkyl)-, and Y is substituted or unsubstituted purinyl.

In another embodiment of the compounds of formula IG-1, L⁴ is —CH(NH(alkyl)alkyl)-, and Y is substituted or unsubstituted purinyl.

In another embodiment of the compounds of formula IG-1, L⁴ is —CH(N(alkyl)₂alkyl)-, and Y is substituted or unsubstituted purinyl.

In another embodiment of the compounds of formula IG-1, L⁴ is —CH(NH₂(CH₂)₃)—, and Y is substituted or unsubstituted purinyl.

In another embodiment of the compounds of formula IG-1, L⁴ is —C(R⁴)₂—, and Y is substituted or unsubstituted thienyl.

In another embodiment of the compounds of formula IG-1, L⁴ is —CH₂—, and Y is substituted or unsubstituted thienyl.

In another embodiment of the compounds of formula IG-1, L⁴ is —CH(CH₃)—, and Y is substituted or unsubstituted thienyl.

In another embodiment of the compounds of formula IG-1, L⁴ is —CH(aminoalkyl)-, and Y is substituted or unsubstituted thienyl.

In another embodiment of the compounds of formula IG-1, L⁴ is —CH(NH₂alkyl)-, and Y is substituted or unsubstituted thienyl.

In another embodiment of the compounds of formula IG-1, L⁴ is —CH(NH(alkyl)alkyl)-, and Y is substituted or unsubstituted thienyl.

In another embodiment of the compounds of formula IG-1, L⁴ is —CH(N(alkyl)₂alkyl)-, and Y is substituted or unsubstituted thienyl.

In another embodiment of the compounds of formula IG-1, L⁴ is —CH(NH₂(CH₂)₃)—, and Y is substituted or unsubstituted thienyl.

In another embodiment of the compounds of formula IG-1, L⁴ is —C(R⁴)₂—, and Y is substituted or unsubstituted furanyl.

In another embodiment of the compounds of formula IG-1, L⁴ is —CH₂—, and Y is substituted or unsubstituted furanyl.

In another embodiment of the compounds of formula IG-1, L⁴ is —CH(CH₃)—, and Y is substituted or unsubstituted furanyl.

In another embodiment of the compounds of formula IG-1, L⁴ is —CH(aminoalkyl)-, and Y is substituted or unsubstituted furanyl.

In another embodiment of the compounds of formula IG-1, L⁴ is —CH(NH₂alkyl)-, and Y is substituted or unsubstituted furanyl.

In another embodiment of the compounds of formula IG-1, L⁴ is —CH(NH(alkyl)alkyl)-, and Y is substituted or unsubstituted furanyl.

In another embodiment of the compounds of formula IG-1, L⁴ is —CH(N(alkyl)₂alkyl)-, and Y is substituted or unsubstituted furanyl.

In another embodiment of the compounds of formula IG-1, L⁴ is —CH(NH₂(CH₂)₃)—, and Y is substituted or unsubstituted furanyl.

In another embodiment of the compounds of formula IG-1, L⁴ is —C(R⁴)₂—, and Y is substituted or unsubstituted pyrrolyl.

In another embodiment of the compounds of formula IG-1, L⁴ is —CH₂—, and Y is substituted or unsubstituted pyrrolyl.

In another embodiment of the compounds of formula IG-1, L⁴ is —CH(CH₃)—, and Y is substituted or unsubstituted pyrrolyl.

In another embodiment of the compounds of formula IG-1, L⁴ is —CH(aminoalkyl)-, and Y is substituted or unsubstituted pyrrolyl.

In another embodiment of the compounds of formula IG-1, L⁴ is —CH(NH₂alkyl)-, and Y is substituted or unsubstituted pyrrolyl.

In another embodiment of the compounds of formula IG-1, L⁴ is —CH(NH(alkyl)alkyl)-, and Y is substituted or unsubstituted pyrrolyl.

In another embodiment of the compounds of formula IG-1, L⁴ is —CH(N(alkyl)₂alkyl)-, and Y is substituted or unsubstituted pyrrolyl.

In another embodiment of the compounds of formula IG-1, L⁴ is —CH(NH₂(CH₂)₃)—, and Y is substituted or unsubstituted pyrrolyl.

In another embodiment of the compounds of formula IG-1, L⁴ is —C(R⁴)₂—, Y is substituted or unsubstituted aryl, and R⁶ is H.

In another embodiment of the compounds of formula IG-1, L⁴ is —CH₂—, Y is substituted or unsubstituted aryl, and R¹ is H.

In another embodiment of the compounds of formula IG-1, L⁴ is —CH(CH₃)—, Y is substituted or unsubstituted aryl, and R¹ is H.

In another embodiment of the compounds of formula IG-1, L⁴ is —CH(aminoalkyl)-, Y is substituted or unsubstituted aryl, and R⁶ is H.

In another embodiment of the compounds of formula IG-1, L⁴ is —CH(NH₂alkyl)-, Y is substituted or unsubstituted aryl, and R⁶ is H.

In another embodiment of the compounds of formula IG-1, L⁴ is —CH(NH(alkyl)alkyl)-, Y is substituted or unsubstituted aryl, and R⁶ is H.

In another embodiment of the compounds of formula IG-1, L⁴ is —CH(N(alkyl)₂alkyl)-, Y is substituted or unsubstituted aryl, and R⁶ is H.

In another embodiment of the compounds of formula IG-1, L⁴ is —CH(NH₂(CH₂)₃)—, Y is substituted or unsubstituted aryl, and R¹ is H.

In another embodiment of the compounds of formula IG-1, L⁴ is —C(R⁴)₂—, Y is substituted or unsubstituted phenyl, and R⁶ is H.

In another embodiment of the compounds of formula IG-1, L⁴ is —CH₂—, Y is substituted or unsubstituted phenyl, and R⁶ is H.

In another embodiment of the compounds of formula IG-1, L⁴ is —CH(CH₃)—, Y is substituted or unsubstituted phenyl, and R⁶ is H.

In another embodiment of the compounds of formula IG-1, L⁴ is —CH(aminoalkyl)-, Y is substituted or unsubstituted phenyl, and R⁶ is H.

In another embodiment of the compounds of formula IG-1, L⁴ is —CH(NH₂alkyl)-, Y is substituted or unsubstituted phenyl, and R⁶ is H.

In another embodiment of the compounds of formula IG-1, L⁴ is —CH(NH(alkyl)alkyl)-, Y is substituted or unsubstituted phenyl, and R⁶ is H.

In another embodiment of the compounds of formula IG-1, L⁴ is —CH(N(alkyl)₂alkyl)-, Y is substituted or unsubstituted phenyl, and R⁶ is H.

In another embodiment of the compounds of formula IG-1, L⁴ is —CH(NH₂(CH₂)₃)—, Y is substituted or unsubstituted phenyl, and R⁶ is H.

In another embodiment of the compounds of formula IG-1, L⁴ is —C(R⁴)₂—, Y is substituted or unsubstituted heteroaryl, and R⁶ is H.

In another embodiment of the compounds of formula IG-1, L⁴ is —CH₂—, Y is substituted or unsubstituted heteroaryl, and R⁶ is H.

In another embodiment of the compounds of formula IG-1, L⁴ is —CH(CH₃)—, Y is substituted or unsubstituted heteroaryl, and R⁶ is H.

In another embodiment of the compounds of formula IG-1, L⁴ is —CH(aminoalkyl)-, Y is substituted or unsubstituted heteroaryl, and R⁶ is H.

In another embodiment of the compounds of formula IG-1, L⁴ is —CH(NH₂alkyl)-, Y is substituted or unsubstituted heteroaryl, and R⁶ is H.

In another embodiment of the compounds of formula IG-1, L⁴ is —CH(NH(alkyl)alkyl)-, Y is substituted or unsubstituted heteroaryl, and R⁶ is H.

In another embodiment of the compounds of formula IG-1, L⁴ is —CH(N(alkyl)₂alkyl)-, Y is substituted or unsubstituted heteroaryl, and R⁶ is H.

In another embodiment of the compounds of formula IG-1, L⁴ is —CH(NH₂(CH₂)₃)—, Y is substituted or unsubstituted heteroaryl, and R⁶ is H.

In another embodiment of the compounds of formula IG-1, L⁴ is —C(R⁴)₂—, Y is substituted or unsubstituted pyridyl, and R⁶ is H.

In another embodiment of the compounds of formula IG-1, L⁴ is —CH₂—, Y is substituted or unsubstituted pyridyl, and R⁶ is H.

In another embodiment of the compounds of formula IG-1, L⁴ is —CH(CH₃)—, Y is substituted or unsubstituted pyridyl, and R⁶ is H.

In another embodiment of the compounds of formula IG-1, L⁴ is —CH(aminoalkyl)-, Y is substituted or unsubstituted pyridyl, and R⁶ is H.

In another embodiment of the compounds of formula IG-1, L⁴ is —CH(NH₂alkyl)-, Y is substituted or unsubstituted pyridyl, and R⁶ is H.

In another embodiment of the compounds of formula IG-1, L⁴ is —CH(NH(alkyl)alkyl)-, Y is substituted or unsubstituted pyridyl, and R⁶ is H.

In another embodiment of the compounds of formula IG-1, L⁴ is —CH(N(alkyl)₂alkyl)-, Y is substituted or unsubstituted pyridyl, and R⁶ is H.

In another embodiment of the compounds of formula IG-1, L⁴ is —CH(NH₂(CH₂)₃)—, Y is substituted or unsubstituted pyridyl, and R⁶ is H.

In another embodiment of the compounds of formula IG-1, L⁴ is —C(R⁴)₂—, Y is substituted or unsubstituted pyrimidyl, and R⁶ is H.

In another embodiment of the compounds of formula IG-1, L⁴ is —CH₂—, Y is substituted or unsubstituted pyrimidyl, and R¹ is H.

In another embodiment of the compounds of formula IG-1, L⁴ is —CH(CH₃)—, Y is substituted or unsubstituted pyrimidyl, and R⁶ is H.

In another embodiment of the compounds of formula IG-1, L⁴ is —CH(aminoalkyl)-, Y is substituted or unsubstituted pyrimidyl, and R⁶ is H.

In another embodiment of the compounds of formula IG-1, L⁴ is —CH(NH₂alkyl)-, Y is substituted or unsubstituted pyrimidyl, and R⁶ is H.

In another embodiment of the compounds of formula IG-1, L⁴ is —CH(NH(alkyl)alkyl)-, Y is substituted or unsubstituted pyrimidyl, and R⁶ is H.

In another embodiment of the compounds of formula IG-1, L⁴ is —CH(N(alkyl)₂alkyl)-, Y is substituted or unsubstituted pyrimidyl, and R⁶ is H.

In another embodiment of the compounds of formula IG-1, L⁴ is —CH(NH₂(CH₂)₃)—, Y is substituted or unsubstituted pyrimidyl, and R⁶ is H.

In another embodiment of the compounds of formula IG-1, L⁴ is —C(R⁴)₂—, Y is substituted or unsubstituted quinolyl, and R⁶ is H.

In another embodiment of the compounds of formula IG-1, L⁴ is —CH₂—, Y is substituted or unsubstituted quinolyl, and R⁶ is H.

In another embodiment of the compounds of formula IG-1, L⁴ is —CH(CH₃)—, Y is substituted or unsubstituted quinolyl, and R⁶ is H.

In another embodiment of the compounds of formula IG-1, L⁴ is —CH(aminoalkyl)-, Y is substituted or unsubstituted quinolyl, and R⁶ is H.

In another embodiment of the compounds of formula IG-1, L⁴ is —CH(NH₂alkyl)-, Y is substituted or unsubstituted quinolyl, and R⁶ is H.

In another embodiment of the compounds of formula IG-1, L⁴ is —CH(NH(alkyl)alkyl)-, Y is substituted or unsubstituted quinolyl, and R¹ is H.

In another embodiment of the compounds of formula IG-1, L⁴ is —CH(N(alkyl)₂alkyl)-, Y is substituted or unsubstituted quinolyl, and R⁶ is H.

In another embodiment of the compounds of formula IG-1, L⁴ is —CH(NH₂(CH₂)₃)—, Y is substituted or unsubstituted quinolyl, and R⁶ is H.

In another embodiment of the compounds of formula IG-1, L⁴ is —C(R⁴)₂—, Y is substituted or unsubstituted indolyl, and R⁶ is H.

In another embodiment of the compounds of formula IG-1, L⁴ is —CH₂—, Y is substituted or unsubstituted indolyl, and R⁶ is H.

In another embodiment of the compounds of formula IG-1, L⁴ is —CH(CH₃)—, Y is substituted or unsubstituted indolyl, and R⁶ is H.

In another embodiment of the compounds of formula IG-1, L⁴ is —CH(aminoalkyl)-, Y is substituted or unsubstituted indolyl, and R⁶ is H.

In another embodiment of the compounds of formula IG-1, L⁴ is —CH(NH₂alkyl)-, Y is substituted or unsubstituted indolyl, and R⁶ is H.

In another embodiment of the compounds of formula IG-1, L⁴ is —CH(NH(alkyl)alkyl)-, Y is substituted or unsubstituted indolyl, and R⁶ is H.

In another embodiment of the compounds of formula IG-1, L⁴ is —CH(N(alkyl)₂alkyl)-, Y is substituted or unsubstituted indolyl, and R⁶ is H.

In another embodiment of the compounds of formula IG-1, L⁴ is —CH(NH₂(CH₂)₃)—, Y is substituted or unsubstituted indolyl, and R⁶ is H.

In another embodiment of the compounds of formula IG-1, L⁴ is —C(R⁴)₂—, Y is substituted or unsubstituted benzimidazolyl, and R⁶ is H.

In another embodiment of the compounds of formula IG-1, L⁴ is —CH₂—, Y is substituted or unsubstituted benzimidazolyl, and R¹ is H.

In another embodiment of the compounds of formula IG-1, L⁴ is —CH(CH₃)—, Y is substituted or unsubstituted benzimidazolyl, and R⁶ is H.

In another embodiment of the compounds of formula IG-1, L⁴ is —CH(aminoalkyl)-, Y is substituted or unsubstituted benzimidazolyl, and R⁶ is H.

In another embodiment of the compounds of formula IG-1, L⁴ is —CH(NH₂alkyl)-, Y is substituted or unsubstituted benzimidazolyl, and R⁶ is H.

In another embodiment of the compounds of formula IG-1, L⁴ is —CH(NH(alkyl)alkyl)-, Y is substituted or unsubstituted benzimidazolyl, and R⁶ is H.

In another embodiment of the compounds of formula IG-1, L⁴ is —CH(N(alkyl)₂alkyl)-, Y is substituted or unsubstituted benzimidazolyl, and R⁶ is H.

In another embodiment of the compounds of formula IG-1, L⁴ is —CH(NH₂(CH₂)₃)—, Y is substituted or unsubstituted benzimidazolyl, and R⁶ is H.

In another embodiment of the compounds of formula IG-1, L⁴ is —C(R⁴)₂—, Y is substituted or unsubstituted purinyl, and R⁶ is H.

In another embodiment of the compounds of formula IG-1, L⁴ is —CH₂—, Y is substituted or unsubstituted purinyl, and R⁶ is H.

In another embodiment of the compounds of formula IG-1, L⁴ is —CH(CH₃)—, Y is substituted or unsubstituted purinyl, and R⁶ is H.

In another embodiment of the compounds of formula IG-1, L⁴ is —CH(aminoalkyl)-, Y is substituted or unsubstituted purinyl, and R⁶ is H.

In another embodiment of the compounds of formula IG-1, L⁴ is —CH(NH₂alkyl)-, Y is substituted or unsubstituted purinyl, and R⁶ is H.

In another embodiment of the compounds of formula IG-1, L⁴ is —CH(NH(alkyl)alkyl)-, Y is substituted or unsubstituted purinyl, and R⁶ is H.

In another embodiment of the compounds of formula IG-1, L⁴ is —CH(N(alkyl)₂alkyl)-, Y is substituted or unsubstituted purinyl, and R⁶ is H.

In another embodiment of the compounds of formula IG-1, L⁴ is —CH(NH₂(CH₂)₃)—, Y is substituted or unsubstituted purinyl, and R¹ is H.

In another embodiment of the compounds of formula IG-1, L⁴ is —C(R⁴)₂—, Y is substituted or unsubstituted thienyl, and R¹ is H.

In another embodiment of the compounds of formula IG-1, L⁴ is —CH₂—, Y is substituted or unsubstituted thienyl, and R⁶ is H.

In another embodiment of the compounds of formula IG-1, L⁴ is —CH(CH₃)—, Y is substituted or unsubstituted thienyl, and R¹ is H.

In another embodiment of the compounds of formula IG-1, L⁴ is —CH(aminoalkyl)-, Y is substituted or unsubstituted thienyl, and R⁶ is H.

In another embodiment of the compounds of formula IG-1, L⁴ is —CH(NH₂alkyl)-, Y is substituted or unsubstituted thienyl, and R⁶ is H.

In another embodiment of the compounds of formula IG-1, L⁴ is —CH(NH(alkyl)alkyl)-, Y is substituted or unsubstituted thienyl, and R¹ is H.

In another embodiment of the compounds of formula IG-1, L⁴ is —CH(N(alkyl)₂alkyl)-, Y is substituted or unsubstituted thienyl, and R⁶ is H.

In another embodiment of the compounds of formula IG-1, L⁴ is —CH(NH₂(CH₂)₃)—, Y is substituted or unsubstituted thienyl, and R¹ is H.

In another embodiment of the compounds of formula IG-1, L⁴ is —C(R⁴)₂—, Y is substituted or unsubstituted furanyl, and R⁶ is H.

In another embodiment of the compounds of formula IG-1, L⁴ is —CH₂—, Y is substituted or unsubstituted furanyl, and R⁶ is H.

In another embodiment of the compounds of formula IG-1, L⁴ is —CH(CH₃)—, Y is substituted or unsubstituted furanyl, and R⁶ is H.

In another embodiment of the compounds of formula IG-1, L⁴ is —CH(aminoalkyl)-, Y is substituted or unsubstituted furanyl, and R⁶ is H.

In another embodiment of the compounds of formula IG-1, L⁴ is —CH(NH₂alkyl)-, Y is substituted or unsubstituted furanyl, and R⁶ is H.

In another embodiment of the compounds of formula IG-1, L⁴ is —CH(NH(alkyl)alkyl)-, Y is substituted or unsubstituted furanyl, and R⁶ is H.

In another embodiment of the compounds of formula IG-1, L⁴ is —CH(N(alkyl)₂alkyl)-, Y is substituted or unsubstituted furanyl, and R⁶ is H.

In another embodiment of the compounds of formula IG-1, L⁴ is —CH(NH₂(CH₂)₃)—, Y is substituted or unsubstituted furanyl, and R⁶ is H.

In another embodiment of the compounds of formula IG-1, L⁴ is —C(R⁴)₂—, Y is substituted or unsubstituted pyrrolyl, and R⁶ is H.

In another embodiment of the compounds of formula IG-1, L⁴ is —CH₂—, Y is substituted or unsubstituted pyrrolyl, and R⁶ is H.

In another embodiment of the compounds of formula IG-1, L⁴ is —CH(CH₃)—, Y is substituted or unsubstituted pyrrolyl, and R⁶ is H.

In another embodiment of the compounds of formula IG-1, L⁴ is —CH(aminoalkyl)-, Y is substituted or unsubstituted pyrrolyl, and R⁶ is H.

In another embodiment of the compounds of formula IG-1, L⁴ is —CH(NH₂alkyl)-, Y is substituted or unsubstituted pyrrolyl, and R¹ is H.

In another embodiment of the compounds of formula IG-1, L⁴ is —CH(NH(alkyl)alkyl)-, Y is substituted or unsubstituted pyrrolyl, and R⁶ is H.

In another embodiment of the compounds of formula IG-1, L⁴ is —CH(N(alkyl)₂alkyl)-, Y is substituted or unsubstituted pyrrolyl, and R¹ is H.

In another embodiment of the compounds of formula IG-1, L⁴ is —CH(NH₂(CH₂)₃)—, Y is substituted or unsubstituted pyrrolyl, and R⁶ is H.

In another embodiment of the compounds of formula IG-1, L⁴ is —C(R⁴)₂—, Y is substituted or unsubstituted aryl, and R⁶ is alkyl.

In another embodiment of the compounds of formula IG-1, L⁴ is —CH₂—, Y is substituted or unsubstituted aryl, and R⁶ is alkyl.

In another embodiment of the compounds of formula IG-1, L⁴ is —CH(CH₃)—, Y is substituted or unsubstituted aryl, and R⁶ is alkyl.

In another embodiment of the compounds of formula IG-1, L⁴ is —CH(aminoalkyl)-, Y is substituted or unsubstituted aryl, and R⁶ is alkyl.

In another embodiment of the compounds of formula IG-1, L⁴ is —CH(NH₂alkyl)-, Y is substituted or unsubstituted aryl, and R⁶ is alkyl.

In another embodiment of the compounds of formula IG-1, L⁴ is —CH(NH(alkyl)alkyl)-, Y is substituted or unsubstituted aryl, and R⁶ is alkyl.

In another embodiment of the compounds of formula IG-1, L⁴ is —CH(N(alkyl)₂alkyl)-, Y is substituted or unsubstituted aryl, and R⁶ is alkyl.

In another embodiment of the compounds of formula IG-1, L⁴ is —CH(NH₂(CH₂)₃)—, Y is substituted or unsubstituted aryl, and R⁶ is alkyl.

In another embodiment of the compounds of formula IG-1, L⁴ is —C(R⁴)₂—, Y is substituted or unsubstituted phenyl, and R⁶ is alkyl.

In another embodiment of the compounds of formula IG-1, L⁴ is —CH₂—, Y is substituted or unsubstituted phenyl, and R⁶ is alkyl.

In another embodiment of the compounds of formula IG-1, L⁴ is —CH(CH₃)—, Y is substituted or unsubstituted phenyl, and R⁶ is alkyl.

In another embodiment of the compounds of formula IG-1, L⁴ is —CH(aminoalkyl)-, Y is substituted or unsubstituted phenyl, and R⁶ is alkyl.

In another embodiment of the compounds of formula IG-1, L⁴ is —CH(NH₂alkyl)-, Y is substituted or unsubstituted phenyl, and R⁶ is alkyl.

In another embodiment of the compounds of formula IG-1, L⁴ is —CH(NH(alkyl)alkyl)-, Y is substituted or unsubstituted phenyl, and R⁶ is alkyl.

In another embodiment of the compounds of formula IG-1, L⁴ is —CH(N(alkyl)₂alkyl)-, Y is substituted or unsubstituted phenyl, and R⁶ is alkyl.

In another embodiment of the compounds of formula IG-1, L⁴ is —CH(NH₂(CH₂)₃)—, Y is substituted or unsubstituted phenyl, and R⁶ is alkyl.

In another embodiment of the compounds of formula IG-1, L⁴ is —C(R⁴)₂—, Y is substituted or unsubstituted heteroaryl, and R⁶ is alkyl.

In another embodiment of the compounds of formula IG-1, L⁴ is —CH₂—, Y is substituted or unsubstituted heteroaryl, and R⁶ is alkyl.

In another embodiment of the compounds of formula IG-1, L⁴ is —CH(CH₃)—, Y is substituted or unsubstituted heteroaryl, and R⁶ is alkyl.

In another embodiment of the compounds of formula IG-1, L⁴ is —CH(aminoalkyl)-, Y is substituted or unsubstituted heteroaryl, and R⁶ is alkyl.

In another embodiment of the compounds of formula IG-1, L⁴ is —CH(NH₂alkyl)-, Y is substituted or unsubstituted heteroaryl, and R⁶ is alkyl.

In another embodiment of the compounds of formula IG-1, L⁴ is —CH(NH(alkyl)alkyl)-, Y is substituted or unsubstituted heteroaryl, and R⁶ is alkyl.

In another embodiment of the compounds of formula IG-1, L⁴ is —CH(N(alkyl)₂alkyl)-, Y is substituted or unsubstituted heteroaryl, and R⁶ is alkyl.

In another embodiment of the compounds of formula IG-1, L⁴ is —CH(NH₂(CH₂)₃)—, Y is substituted or unsubstituted heteroaryl, and R⁶ is alkyl.

In another embodiment of the compounds of formula IG-1, L⁴ is —C(R⁴)₂—, Y is substituted or unsubstituted pyridyl, and R⁶ is alkyl.

In another embodiment of the compounds of formula IG-1, L⁴ is —CH₂—, Y is substituted or unsubstituted pyridyl, and R⁶ is alkyl.

In another embodiment of the compounds of formula IG-1, L⁴ is —CH(CH₃)—, Y is substituted or unsubstituted pyridyl, and R⁶ is alkyl.

In another embodiment of the compounds of formula IG-1, L⁴ is —CH(aminoalkyl)-, Y is substituted or unsubstituted pyridyl, and R⁶ is alkyl.

In another embodiment of the compounds of formula IG-1, L⁴ is —CH(NH₂alkyl)-, Y is substituted or unsubstituted pyridyl, and R⁶ is alkyl.

In another embodiment of the compounds of formula IG-1, L⁴ is —CH(NH(alkyl)alkyl)-, Y is substituted or unsubstituted pyridyl, and R⁶ is alkyl.

In another embodiment of the compounds of formula IG-1, L⁴ is —CH(N(alkyl)₂alkyl)-, Y is substituted or unsubstituted pyridyl, and R⁶ is alkyl.

In another embodiment of the compounds of formula IG-1, L⁴ is —CH(NH₂(CH₂)₃)—, Y is substituted or unsubstituted pyridyl, and R⁶ is alkyl.

In another embodiment of the compounds of formula IG-1, L⁴ is —C(R⁴)₂—, Y is substituted or unsubstituted pyrimidyl, and R⁶ is alkyl.

In another embodiment of the compounds of formula IG-1, L⁴ is —CH₂—, Y is substituted or unsubstituted pyrimidyl, and R⁶ is alkyl.

In another embodiment of the compounds of formula IG-1, L⁴ is —CH(CH₃)—, Y is substituted or unsubstituted pyrimidyl, and R⁶ is alkyl.

In another embodiment of the compounds of formula IG-1, L⁴ is —CH(aminoalkyl)-, Y is substituted or unsubstituted pyrimidyl, and R⁶ is alkyl.

In another embodiment of the compounds of formula IG-1, L⁴ is —CH(NH₂alkyl)-, Y is substituted or unsubstituted pyrimidyl, and R⁶ is alkyl.

In another embodiment of the compounds of formula IG-1, L⁴ is —CH(NH(alkyl)alkyl)-, Y is substituted or unsubstituted pyrimidyl, and R⁶ is alkyl.

In another embodiment of the compounds of formula IG-1, L⁴ is —CH(N(alkyl)₂alkyl)-, Y is substituted or unsubstituted pyrimidyl, and R⁶ is alkyl.

In another embodiment of the compounds of formula IG-1, L⁴ is —CH(NH₂(CH₂)₃)—, Y is substituted or unsubstituted pyrimidyl, and R⁶ is alkyl.

In another embodiment of the compounds of formula IG-1, L⁴ is —C(R⁴)₂—, Y is substituted or unsubstituted quinolyl, and R⁶ is alkyl.

In another embodiment of the compounds of formula IG-1, L⁴ is —CH₂—, Y is substituted or unsubstituted quinolyl, and R⁶ is alkyl.

In another embodiment of the compounds of formula IG-1, L⁴ is —CH(CH₃)—, Y is substituted or unsubstituted quinolyl, and R⁶ is alkyl.

In another embodiment of the compounds of formula IG-1, L⁴ is —CH(aminoalkyl)-, Y is substituted or unsubstituted quinolyl, and R⁶ is alkyl.

In another embodiment of the compounds of formula IG-1, L⁴ is —CH(NH₂alkyl)-, Y is substituted or unsubstituted quinolyl, and R⁶ is alkyl.

In another embodiment of the compounds of formula IG-1, L⁴ is —CH(NH(alkyl)alkyl)-, Y is substituted or unsubstituted quinolyl, and R⁶ is alkyl.

In another embodiment of the compounds of formula IG-1, L⁴ is —CH(N(alkyl)₂alkyl)-, Y is substituted or unsubstituted quinolyl, and R⁶ is alkyl.

In another embodiment of the compounds of formula IG-1, L⁴ is —CH(NH₂(CH₂)₃)—, Y is substituted or unsubstituted quinolyl, and R⁶ is alkyl.

In another embodiment of the compounds of formula IG-1, L⁴ is —C(R⁴)₂—, Y is substituted or unsubstituted indolyl, and R⁶ is alkyl.

In another embodiment of the compounds of formula IG-1, L⁴ is —CH₂—, Y is substituted or unsubstituted indolyl, and R⁶ is alkyl.

In another embodiment of the compounds of formula IG-1, L⁴ is —CH(CH₃)—, Y is substituted or unsubstituted indolyl, and R⁶ is alkyl.

In another embodiment of the compounds of formula IG-1, L⁴ is —CH(aminoalkyl)-, Y is substituted or unsubstituted indolyl, and R⁶ is alkyl.

In another embodiment of the compounds of formula IG-1, L⁴ is —CH(NH₂alkyl)-, Y is substituted or unsubstituted indolyl, and R⁶ is alkyl.

In another embodiment of the compounds of formula IG-1, L⁴ is —CH(NH(alkyl)alkyl)-, Y is substituted or unsubstituted indolyl, and R⁶ is alkyl.

In another embodiment of the compounds of formula IG-1, L⁴ is —CH(N(alkyl)₂alkyl)-, Y is substituted or unsubstituted indolyl, and R⁶ is alkyl.

In another embodiment of the compounds of formula IG-1, L⁴ is —CH(NH₂(CH₂)₃)—, Y is substituted or unsubstituted indolyl, and R⁶ is alkyl.

In another embodiment of the compounds of formula IG-1, L⁴ is —C(R⁴)₂—, Y is substituted or unsubstituted benzimidazolyl, and R⁶ is alkyl.

In another embodiment of the compounds of formula IG-1, L⁴ is —CH₂—, Y is substituted or unsubstituted benzimidazolyl, and R⁶ is alkyl.

In another embodiment of the compounds of formula IG-1, L⁴ is —CH(CH₃)—, Y is substituted or unsubstituted benzimidazolyl, and R⁶ is alkyl.

In another embodiment of the compounds of formula IG-1, L⁴ is —CH(aminoalkyl)-, Y is substituted or unsubstituted benzimidazolyl, and R⁶ is alkyl.

In another embodiment of the compounds of formula IG-1, L⁴ is —CH(NH₂alkyl)-, Y is substituted or unsubstituted benzimidazolyl, and R⁶ is alkyl.

In another embodiment of the compounds of formula IG-1, L⁴ is —CH(NH(alkyl)alkyl)-, Y is substituted or unsubstituted benzimidazolyl, and R⁶ is alkyl.

In another embodiment of the compounds of formula IG-1, L⁴ is —CH(N(alkyl)₂alkyl)-, Y is substituted or unsubstituted benzimidazolyl, and R⁶ is alkyl.

In another embodiment of the compounds of formula IG-1, L⁴ is —CH(NH₂(CH₂)₃)—, Y is substituted or unsubstituted benzimidazolyl, and R⁶ is alkyl.

In another embodiment of the compounds of formula IG-1, L⁴ is —C(R⁴)₂—, Y is substituted or unsubstituted purinyl, and R⁶ is alkyl.

In another embodiment of the compounds of formula IG-1, L⁴ is —CH₂—, Y is substituted or unsubstituted purinyl, and R⁶ is alkyl.

In another embodiment of the compounds of formula IG-1, L⁴ is —CH(CH₃)—, Y is substituted or unsubstituted purinyl, and R⁶ is alkyl.

In another embodiment of the compounds of formula IG-1, L⁴ is —CH(aminoalkyl)-, Y is substituted or unsubstituted purinyl, and R⁶ is alkyl.

In another embodiment of the compounds of formula IG-1, L⁴ is —CH(NH₂alkyl)-, Y is substituted or unsubstituted purinyl, and R⁶ is alkyl.

In another embodiment of the compounds of formula IG-1, L⁴ is —CH(NH(alkyl)alkyl)-, Y is substituted or unsubstituted purinyl, and R⁶ is alkyl.

In another embodiment of the compounds of formula IG-1, L⁴ is —CH(N(alkyl)₂alkyl)-, Y is substituted or unsubstituted purinyl, and R⁶ is alkyl.

In another embodiment of the compounds of formula IG-1, L⁴ is —CH(NH₂(CH₂)₃)—, Y is substituted or unsubstituted purinyl, and R⁶ is alkyl.

In another embodiment of the compounds of formula IG-1, L⁴ is —C(R⁴)₂—, Y is substituted or unsubstituted thienyl, and R⁶ is alkyl.

In another embodiment of the compounds of formula IG-1, L⁴ is —CH₂—, Y is substituted or unsubstituted thienyl, and R⁶ is alkyl.

In another embodiment of the compounds of formula IG-1, L⁴ is —CH(CH₃)—, Y is substituted or unsubstituted thienyl, and R⁶ is alkyl.

In another embodiment of the compounds of formula IG-1, L⁴ is —CH(aminoalkyl)-, Y is substituted or unsubstituted thienyl, and R⁶ is alkyl.

In another embodiment of the compounds of formula IG-1, L⁴ is —CH(NH₂alkyl)-, Y is substituted or unsubstituted thienyl, and R⁶ is alkyl.

In another embodiment of the compounds of formula IG-1, L⁴ is —CH(NH(alkyl)alkyl)-, Y is substituted or unsubstituted thienyl, and R⁶ is alkyl.

In another embodiment of the compounds of formula IG-1, L⁴ is —CH(N(alkyl)₂alkyl)-, Y is substituted or unsubstituted thienyl, and R⁶ is alkyl.

In another embodiment of the compounds of formula IG-1, L⁴ is —CH(NH₂(CH₂)₃)—, Y is substituted or unsubstituted thienyl, and R⁶ is alkyl.

In another embodiment of the compounds of formula IG-1, L⁴ is —C(R⁴)₂—, Y is substituted or unsubstituted furanyl, and R⁶ is alkyl.

In another embodiment of the compounds of formula IG-1, L⁴ is —CH₂—, Y is substituted or unsubstituted furanyl, and R⁶ is alkyl.

In another embodiment of the compounds of formula IG-1, L⁴ is —CH(CH₃)—, Y is substituted or unsubstituted furanyl, and R⁶ is alkyl.

In another embodiment of the compounds of formula IG-1, L⁴ is —CH(aminoalkyl)-, Y is substituted or unsubstituted furanyl, and R⁶ is alkyl.

In another embodiment of the compounds of formula IG-1, L⁴ is —CH(NH₂alkyl)-, Y is substituted or unsubstituted furanyl, and R⁶ is alkyl.

In another embodiment of the compounds of formula IG-1, L⁴ is —CH(NH(alkyl)alkyl)-, Y is substituted or unsubstituted furanyl, and R⁶ is alkyl.

In another embodiment of the compounds of formula IG-1, L⁴ is —CH(N(alkyl)₂alkyl)-, Y is substituted or unsubstituted furanyl, and R⁶ is alkyl.

In another embodiment of the compounds of formula IG-1, L⁴ is —CH(NH₂(CH₂)₃)—, Y is substituted or unsubstituted furanyl, and R⁶ is alkyl.

In another embodiment of the compounds of formula IG-1, L⁴ is —C(R⁴)₂—, Y is substituted or unsubstituted pyrrolyl, and R⁶ is alkyl.

In another embodiment of the compounds of formula IG-1, L⁴ is —CH₂—, Y is substituted or unsubstituted pyrrolyl, and R⁶ is alkyl.

In another embodiment of the compounds of formula IG-1, L⁴ is —CH(CH₃)—, Y is substituted or unsubstituted pyrrolyl, and R⁶ is alkyl.

In another embodiment of the compounds of formula IG-1, L⁴ is —CH(aminoalkyl)-, Y is substituted or unsubstituted pyrrolyl, and R⁶ is alkyl.

In another embodiment of the compounds of formula IG-1, L⁴ is —CH(NH₂alkyl)-, Y is substituted or unsubstituted pyrrolyl, and R⁶ is alkyl.

In another embodiment of the compounds of formula IG-1, L⁴ is —CH(NH(alkyl)alkyl)-, Y is substituted or unsubstituted pyrrolyl, and R⁶ is alkyl.

In another embodiment of the compounds of formula IG-1, L⁴ is —CH(N(alkyl)₂alkyl)-, Y is substituted or unsubstituted pyrrolyl, and R⁶ is alkyl.

In another embodiment of the compounds of formula IG-1, L⁴ is —CH(NH₂(CH₂)₃)—, Y is substituted or unsubstituted pyrrolyl, and R⁶ is alkyl.

In another embodiment of the compounds of formula IG-1, L⁴ is —C(R⁴)₂—, Y is substituted or unsubstituted aryl, and R⁶ is substituted or unsubstituted arylalkyl.

In another embodiment of the compounds of formula IG-1, L⁴ is —CH₂—, Y is substituted or unsubstituted aryl, and R⁶ is substituted or unsubstituted arylalkyl.

In another embodiment of the compounds of formula IG-1, L⁴ is —CH(CH₃)—, Y is substituted or unsubstituted aryl, and R⁶ is substituted or unsubstituted arylalkyl.

In another embodiment of the compounds of formula IG-1, L⁴ is —CH(aminoalkyl)-, Y is substituted or unsubstituted aryl, and R⁶ is substituted or unsubstituted arylalkyl.

In another embodiment of the compounds of formula IG-1, L⁴ is —CH(NH₂alkyl)-, Y is substituted or unsubstituted aryl, and R⁶ is substituted or unsubstituted arylalkyl.

In another embodiment of the compounds of formula IG-1, L⁴ is —CH(NH(alkyl)alkyl)-, Y is substituted or unsubstituted aryl, and R⁶ is substituted or unsubstituted arylalkyl.

In another embodiment of the compounds of formula IG-1, L⁴ is —CH(N(alkyl)₂alkyl)-, Y is substituted or unsubstituted aryl, and R⁶ is substituted or unsubstituted arylalkyl.

In another embodiment of the compounds of formula IG-1, L⁴ is —CH(NH₂(CH₂)₃)—, Y is substituted or unsubstituted aryl, and R⁶ is substituted or unsubstituted arylalkyl.

In another embodiment of the compounds of formula IG-1, L⁴ is —C(R⁴)₂—, Y is substituted or unsubstituted phenyl, and R⁶ is substituted or unsubstituted arylalkyl.

In another embodiment of the compounds of formula IG-1, L⁴ is —CH₂—, Y is substituted or unsubstituted phenyl, and R⁶ is substituted or unsubstituted arylalkyl.

In another embodiment of the compounds of formula IG-1, L⁴ is —CH(CH₃)—, Y is substituted or unsubstituted phenyl, and R⁶ is substituted or unsubstituted arylalkyl.

In another embodiment of the compounds of formula IG-1, L⁴ is —CH(aminoalkyl)-, Y is substituted or unsubstituted phenyl, and R⁶ is substituted or unsubstituted arylalkyl.

In another embodiment of the compounds of formula IG-1, L⁴ is —CH(NH₂alkyl)-, Y is substituted or unsubstituted phenyl, and R⁶ is substituted or unsubstituted arylalkyl.

In another embodiment of the compounds of formula IG-1, L⁴ is —CH(NH(alkyl)alkyl)-, Y is substituted or unsubstituted phenyl, and R⁶ is substituted or unsubstituted arylalkyl.

In another embodiment of the compounds of formula IG-1, L⁴ is —CH(N(alkyl)₂alkyl)-, Y is substituted or unsubstituted phenyl, and R⁶ is substituted or unsubstituted arylalkyl.

In another embodiment of the compounds of formula IG-1, L⁴ is —CH(NH₂(CH₂)₃)—, Y is substituted or unsubstituted phenyl, and R⁶ is substituted or unsubstituted arylalkyl.

In another embodiment of the compounds of formula IG-1, L⁴ is —C(R⁴)₂—, Y is substituted or unsubstituted heteroaryl, and R⁶ is substituted or unsubstituted arylalkyl.

In another embodiment of the compounds of formula IG-1, L⁴ is —CH₂—, Y is substituted or unsubstituted heteroaryl, and R⁶ is substituted or unsubstituted arylalkyl.

In another embodiment of the compounds of formula IG-1, L⁴ is —CH(CH₃)—, Y is substituted or unsubstituted heteroaryl, and R⁶ is substituted or unsubstituted arylalkyl.

In another embodiment of the compounds of formula IG-1, L⁴ is —CH(aminoalkyl)-, Y is substituted or unsubstituted heteroaryl, and R⁶ is substituted or unsubstituted arylalkyl.

In another embodiment of the compounds of formula IG-1, L⁴ is —CH(NH₂alkyl)-, Y is substituted or unsubstituted heteroaryl, and R⁶ is substituted or unsubstituted arylalkyl.

In another embodiment of the compounds of formula IG-1, L⁴ is —CH(NH(alkyl)alkyl)-, Y is substituted or unsubstituted heteroaryl, and R⁶ is substituted or unsubstituted arylalkyl.

In another embodiment of the compounds of formula IG-1, L⁴ is —CH(N(alkyl)₂alkyl)-, Y is substituted or unsubstituted heteroaryl, and R⁶ is substituted or unsubstituted arylalkyl.

In another embodiment of the compounds of formula IG-1, L⁴ is —CH(NH₂(CH₂)₃)—, Y is substituted or unsubstituted heteroaryl, and R⁶ is substituted or unsubstituted arylalkyl.

In another embodiment of the compounds of formula IG-1, L⁴ is —C(R⁴)₂—, Y is substituted or unsubstituted pyridyl, and R⁶ is substituted or unsubstituted arylalkyl.

In another embodiment of the compounds of formula IG-1, L⁴ is —CH₂—, Y is substituted or unsubstituted pyridyl, and R⁶ is substituted or unsubstituted arylalkyl.

In another embodiment of the compounds of formula IG-1, L⁴ is —CH(CH₃)—, Y is substituted or unsubstituted pyridyl, and R⁶ is substituted or unsubstituted arylalkyl.

In another embodiment of the compounds of formula IG-1, L⁴ is —CH(aminoalkyl)-, Y is substituted or unsubstituted pyridyl, and R⁶ is substituted or unsubstituted arylalkyl.

In another embodiment of the compounds of formula IG-1, L⁴ is —CH(NH₂alkyl)-, Y is substituted or unsubstituted pyridyl, and R⁶ is substituted or unsubstituted arylalkyl.

In another embodiment of the compounds of formula IG-1, L⁴ is —CH(NH(alkyl)alkyl)-, Y is substituted or unsubstituted pyridyl, and R⁶ is substituted or unsubstituted arylalkyl.

In another embodiment of the compounds of formula IG-1, L⁴ is —CH(N(alkyl)₂alkyl)-, Y is substituted or unsubstituted pyridyl, and R⁶ is substituted or unsubstituted arylalkyl.

In another embodiment of the compounds of formula IG-1, L⁴ is —CH(NH₂(CH₂)₃)—, Y is substituted or unsubstituted pyridyl, and R⁶ is substituted or unsubstituted arylalkyl.

In another embodiment of the compounds of formula IG-1, L⁴ is —C(R⁴)₂—, Y is substituted or unsubstituted pyrimidyl, and R⁶ is substituted or unsubstituted arylalkyl.

In another embodiment of the compounds of formula IG-1, L⁴ is —CH₂—, Y is substituted or unsubstituted pyrimidyl, and R⁶ is substituted or unsubstituted arylalkyl.

In another embodiment of the compounds of formula IG-1, L⁴ is —CH(CH₃)—, Y is substituted or unsubstituted pyrimidyl, and R⁶ is substituted or unsubstituted arylalkyl.

In another embodiment of the compounds of formula IG-1, L⁴ is —CH(aminoalkyl)-, Y is substituted or unsubstituted pyrimidyl, and R⁶ is substituted or unsubstituted arylalkyl.

In another embodiment of the compounds of formula IG-1, L⁴ is —CH(NH₂alkyl)-, Y is substituted or unsubstituted pyrimidyl, and R⁶ is substituted or unsubstituted arylalkyl.

In another embodiment of the compounds of formula IG-1, L⁴ is —CH(NH(alkyl)alkyl)-, Y is substituted or unsubstituted pyrimidyl, and R⁶ is substituted or unsubstituted arylalkyl.

In another embodiment of the compounds of formula IG-1, L⁴ is —CH(N(alkyl)₂alkyl)-, Y is substituted or unsubstituted pyrimidyl, and R⁶ is substituted or unsubstituted arylalkyl.

In another embodiment of the compounds of formula IG-1, L⁴ is —CH(NH₂(CH₂)₃)—, Y is substituted or unsubstituted pyrimidyl, and R¹ is substituted or unsubstituted arylalkyl.

In another embodiment of the compounds of formula IG-1, L⁴ is —C(R⁴)₂—, Y is substituted or unsubstituted quinolyl, and R⁶ is substituted or unsubstituted arylalkyl.

In another embodiment of the compounds of formula IG-1, L⁴ is —CH₂—, Y is substituted or unsubstituted quinolyl, and R⁶ is substituted or unsubstituted arylalkyl.

In another embodiment of the compounds of formula IG-1, L⁴ is —CH(CH₃)—, Y is substituted or unsubstituted quinolyl, and R⁶ is substituted or unsubstituted arylalkyl.

In another embodiment of the compounds of formula IG-1, L⁴ is —CH(aminoalkyl)-, Y is substituted or unsubstituted quinolyl, and R⁶ is substituted or unsubstituted arylalkyl.

In another embodiment of the compounds of formula IG-1, L⁴ is —CH(NH₂alkyl)-, Y is substituted or unsubstituted quinolyl, and R⁶ is substituted or unsubstituted arylalkyl.

In another embodiment of the compounds of formula IG-1, L⁴ is —CH(NH(alkyl)alkyl)-, Y is substituted or unsubstituted quinolyl, and R⁶ is substituted or unsubstituted arylalkyl.

In another embodiment of the compounds of formula IG-1, L⁴ is —CH(N(alkyl)₂alkyl)-, Y is substituted or unsubstituted quinolyl, and R⁶ is substituted or unsubstituted arylalkyl.

In another embodiment of the compounds of formula IG-1, L⁴ is —CH(NH₂(CH₂)₃)—, Y is substituted or unsubstituted quinolyl, and R⁶ is substituted or unsubstituted arylalkyl.

In another embodiment of the compounds of formula IG-1, L⁴ is —C(R⁴)₂—, Y is substituted or unsubstituted indolyl, and R⁶ is substituted or unsubstituted arylalkyl.

In another embodiment of the compounds of formula IG-1, L⁴ is —CH₂—, Y is substituted or unsubstituted indolyl, and R¹ is substituted or unsubstituted arylalkyl.

In another embodiment of the compounds of formula IG-1, L⁴ is —CH(CH₃)—, Y is substituted or unsubstituted indolyl, and R⁶ is substituted or unsubstituted arylalkyl.

In another embodiment of the compounds of formula IG-1, L⁴ is —CH(aminoalkyl)-, Y is substituted or unsubstituted indolyl, and R⁶ is substituted or unsubstituted arylalkyl.

In another embodiment of the compounds of formula IG-1, L⁴ is —CH(NH₂alkyl)-, Y is substituted or unsubstituted indolyl, and R⁶ is substituted or unsubstituted arylalkyl.

In another embodiment of the compounds of formula IG-1, L⁴ is —CH(NH(alkyl)alkyl)-, Y is substituted or unsubstituted indolyl, and R⁶ is substituted or unsubstituted arylalkyl.

In another embodiment of the compounds of formula IG-1, L⁴ is —CH(N(alkyl)₂alkyl)-, Y is substituted or unsubstituted indolyl, and R⁶ is substituted or unsubstituted arylalkyl.

In another embodiment of the compounds of formula IG-1, L⁴ is —CH(NH₂(CH₂)₃)—, Y is substituted or unsubstituted indolyl, and R⁶ is substituted or unsubstituted arylalkyl.

In another embodiment of the compounds of formula IG-1, L⁴ is —C(R⁴)₂—, Y is substituted or unsubstituted benzimidazolyl, and R⁶ is substituted or unsubstituted arylalkyl.

In another embodiment of the compounds of formula IG-1, L⁴ is —CH₂—, Y is substituted or unsubstituted benzimidazolyl, and R⁶ is substituted or unsubstituted arylalkyl.

In another embodiment of the compounds of formula IG-1, L⁴ is —CH(CH₃)—, Y is substituted or unsubstituted benzimidazolyl, and R⁶ is substituted or unsubstituted arylalkyl.

In another embodiment of the compounds of formula IG-1, L⁴ is —CH(aminoalkyl)-, Y is substituted or unsubstituted benzimidazolyl, and R⁶ is substituted or unsubstituted arylalkyl.

In another embodiment of the compounds of formula IG-1, L⁴ is —CH(NH₂alkyl)-, Y is substituted or unsubstituted benzimidazolyl, and R⁶ is substituted or unsubstituted arylalkyl.

In another embodiment of the compounds of formula IG-1, L⁴ is —CH(NH(alkyl)alkyl)-, Y is substituted or unsubstituted benzimidazolyl, and R⁶ is substituted or unsubstituted arylalkyl.

In another embodiment of the compounds of formula IG-1, L⁴ is —CH(N(alkyl)₂alkyl)-, Y is substituted or unsubstituted benzimidazolyl, and R⁶ is substituted or unsubstituted arylalkyl.

In another embodiment of the compounds of formula IG-1, L⁴ is —CH(NH₂(CH₂)₃)—, Y is substituted or unsubstituted benzimidazolyl, and R⁶ is substituted or unsubstituted arylalkyl.

In another embodiment of the compounds of formula IG-1, L⁴ is —C(R⁴)₂—, Y is substituted or unsubstituted purinyl, and R⁶ is substituted or unsubstituted arylalkyl.

In another embodiment of the compounds of formula IG-1, L⁴ is —CH₂—, Y is substituted or unsubstituted purinyl, and R⁶ is substituted or unsubstituted arylalkyl.

In another embodiment of the compounds of formula IG-1, L⁴ is —CH(CH₃)—, Y is substituted or unsubstituted purinyl, and R⁶ is substituted or unsubstituted arylalkyl.

In another embodiment of the compounds of formula IG-1, L⁴ is —CH(aminoalkyl)-, Y is substituted or unsubstituted purinyl, and R⁶ is substituted or unsubstituted arylalkyl.

In another embodiment of the compounds of formula IG-1, L⁴ is —CH(NH₂alkyl)-, Y is substituted or unsubstituted purinyl, and R⁶ is substituted or unsubstituted arylalkyl.

In another embodiment of the compounds of formula IG-1, L⁴ is —CH(NH(alkyl)alkyl)-, Y is substituted or unsubstituted purinyl, and R⁶ is substituted or unsubstituted arylalkyl.

In another embodiment of the compounds of formula IG-1, L⁴ is —CH(N(alkyl)₂alkyl)-, Y is substituted or unsubstituted purinyl, and R⁶ is substituted or unsubstituted arylalkyl.

In another embodiment of the compounds of formula IG-1, L⁴ is —CH(NH₂(CH₂)₃)—, Y is substituted or unsubstituted purinyl, and R⁶ is substituted or unsubstituted arylalkyl.

In another embodiment of the compounds of formula IG-1, L⁴ is —C(R⁴)₂—, Y is substituted or unsubstituted thienyl, and R⁶ is substituted or unsubstituted arylalkyl.

In another embodiment of the compounds of formula IG-1, L⁴ is —CH₂—, Y is substituted or unsubstituted thienyl, and R⁶ is substituted or unsubstituted arylalkyl.

In another embodiment of the compounds of formula IG-1, L⁴ is —CH(CH₃)—, Y is substituted or unsubstituted thienyl, and R⁶ is substituted or unsubstituted arylalkyl.

In another embodiment of the compounds of formula IG-1, L⁴ is —CH(aminoalkyl)-, Y is substituted or unsubstituted thienyl, and R⁶ is substituted or unsubstituted arylalkyl.

In another embodiment of the compounds of formula IG-1, L⁴ is —CH(NH₂alkyl)-, Y is substituted or unsubstituted thienyl, and R⁶ is substituted or unsubstituted arylalkyl.

In another embodiment of the compounds of formula IG-1, L⁴ is —CH(NH(alkyl)alkyl)-, Y is substituted or unsubstituted thienyl, and R⁶ is substituted or unsubstituted arylalkyl.

In another embodiment of the compounds of formula IG-1, L⁴ is —CH(N(alkyl)₂alkyl)-, Y is substituted or unsubstituted thienyl, and R⁶ is substituted or unsubstituted arylalkyl.

In another embodiment of the compounds of formula IG-1, L⁴ is —CH(NH₂(CH₂)₃)—, Y is substituted or unsubstituted thienyl, and R⁶ is substituted or unsubstituted arylalkyl.

In another embodiment of the compounds of formula IG-1, L⁴ is —C(R⁴)₂—, Y is substituted or unsubstituted furanyl, and R⁶ is substituted or unsubstituted arylalkyl.

In another embodiment of the compounds of formula IG-1, L⁴ is —CH₂—, Y is substituted or unsubstituted furanyl, and R⁶ is substituted or unsubstituted arylalkyl.

In another embodiment of the compounds of formula IG-1, L⁴ is —CH(CH₃)—, Y is substituted or unsubstituted furanyl, and R⁶ is substituted or unsubstituted arylalkyl.

In another embodiment of the compounds of formula IG-1, L⁴ is —CH(aminoalkyl)-, Y is substituted or unsubstituted furanyl, and R⁶ is substituted or unsubstituted arylalkyl.

In another embodiment of the compounds of formula IG-1, L⁴ is —CH(NH₂alkyl)-, Y is substituted or unsubstituted furanyl, and R⁶ is substituted or unsubstituted arylalkyl.

In another embodiment of the compounds of formula IG-1, L⁴ is —CH(NH(alkyl)alkyl)-, Y is substituted or unsubstituted furanyl, and R⁶ is substituted or unsubstituted arylalkyl.

In another embodiment of the compounds of formula IG-1, L⁴ is —CH(N(alkyl)₂alkyl)-, Y is substituted or unsubstituted furanyl, and R⁶ is substituted or unsubstituted arylalkyl.

In another embodiment of the compounds of formula IG-1, L⁴ is —CH(NH₂(CH₂)₃)—, Y is substituted or unsubstituted furanyl, and R⁶ is substituted or unsubstituted arylalkyl.

In another embodiment of the compounds of formula IG-1, L⁴ is —C(R⁴)₂—, Y is substituted or unsubstituted pyrrolyl, and R⁶ is substituted or unsubstituted arylalkyl.

In another embodiment of the compounds of formula IG-1, L⁴ is —CH₂—, Y is substituted or unsubstituted pyrrolyl, and R⁶ is substituted or unsubstituted arylalkyl.

In another embodiment of the compounds of formula IG-1, L⁴ is —CH(CH₃)—, Y is substituted or unsubstituted pyrrolyl, and R⁶ is substituted or unsubstituted arylalkyl.

In another embodiment of the compounds of formula IG-1, L⁴ is —CH(aminoalkyl)-, Y is substituted or unsubstituted pyrrolyl, and R⁶ is substituted or unsubstituted arylalkyl.

In another embodiment of the compounds of formula IG-1, L⁴ is —CH(NH₂alkyl)-, Y is substituted or unsubstituted pyrrolyl, and R⁶ is substituted or unsubstituted arylalkyl.

In another embodiment of the compounds of formula IG-1, L⁴ is —CH(NH(alkyl)alkyl)-, Y is substituted or unsubstituted pyrrolyl, and R⁶ is substituted or unsubstituted arylalkyl.

In another embodiment of the compounds of formula IG-1, L⁴ is —CH(N(alkyl)₂alkyl)-, Y is substituted or unsubstituted pyrrolyl, and R⁶ is substituted or unsubstituted arylalkyl.

In another embodiment of the compounds of formula IG-1, L⁴ is —CH(NH₂(CH₂)₃)—, Y is substituted or unsubstituted pyrrolyl, and R⁶ is substituted or unsubstituted arylalkyl.

In another embodiment of the compounds of formula IG-1, R² is H.

In another embodiment of the compounds of formula IG-1, R² is alkyl.

In another embodiment of the compounds of formula IG-1, R² is methyl.

In another embodiment of the compounds of formula IG-1, R² is ethyl.

In another embodiment of the compounds of formula IG-1, R² is propyl.

In another embodiment of the compounds of formula IG-1, R² is butyl.

In another embodiment of the compounds of formula IG-1, R² is haloalkyl.

In another embodiment of the compounds of formula IG-1, R² is CF₃.

In another embodiment of the compounds of formula IG-1, R² is —CH₂CF₃.

In another embodiment of the compounds of formula IG-1, L⁴ is a covalent bond and Y is substituted or unsubstituted cycloalkyl.

In another embodiment of the compounds of formula IG-1, L⁴ is a covalent bond and Y is substituted or unsubstituted 5,6,7,8-tetrahydroquinolin-8-yl.

In another embodiment of the compounds of formula IG-1, L⁴ is a covalent bond, Y is substituted or unsubstituted cycloalkyl, and R⁶ is substituted or unsubstituted aminoalkyl.

In another embodiment of the compounds of formula IG-1, L⁴ is a covalent bond, Y is substituted or unsubstituted cycloalkyl, and R⁶ is aminobutyl.

In another embodiment of the compounds of formula IG-1, L⁴ is a covalent bond. Y is substituted or unsubstituted 5,6,7,8-tetrahydroquinolin-8-yl, and R⁶ is aminoalkyl.

In another embodiment of the compounds of formula IG-1, L⁴ is a covalent bond. Y is substituted or unsubstituted 5,6,7,8-tetrahydroquinolin-8-yl, and R⁶ is aminobutyl.

In another embodiment of the compounds of formula IG-1, L⁴ is a covalent bond, Y is substituted or unsubstituted cycloalkyl, and R⁶ is alkyl.

In another embodiment of the compounds of formula IG-1, L⁴ is a covalent bond. Y is substituted or unsubstituted 5,6,7,8-tetrahydroquinolin-8-yl, and R⁶ is alkyl.

In another embodiment of the compounds of formula IG-1, L⁴ is a covalent bond, Y is substituted or unsubstituted cycloalkyl, and R⁶ is methyl.

In another embodiment of the compounds of formula IG-1, L⁴ is a covalent bond. Y is substituted or unsubstituted 5,6,7,8-tetrahydroquinolin-8-yl, and R⁶ is methyl.

In another embodiment, the compounds of the present invention, or pharmaceutically acceptable salts, solvates, prodrugs, tautomers, or esters thereof, have the structure of formula (IH):

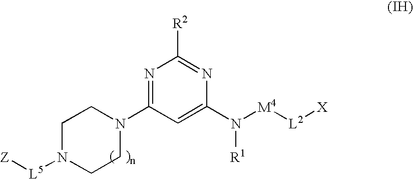

wherein R¹, R², M⁴, L², L⁵, n, X and Z are as defined herein.

In another embodiment of the compounds of formula IH, R¹ and R⁴ are H; R⁵ is selected from the group consisting of H and substituted or unsubstituted benzyl; X and Z are each substituted or unsubstituted phenyl; and n is 1.

In another embodiment of the compounds of formula IH, M⁴ is —C(R⁴)₂—; L² is a covalent bond; X is selected from the group consisting of substituted or unsubstituted aryl and substituted or unsubstituted heteroaryl; R¹ is selected from the group consisting of H, alkyl, substituted or unsubstituted arylalkyl, substituted or unsubstituted heteroarylalkyl, and substituted or unsubstituted cycloalkyl; and each R⁴ is independently selected from the group consisting of H, alkyl, and substituted or unsubstituted aryl.

In another embodiment of the compounds of formula IH, M⁴ is selected from the group consisting of —CH₂—, —CH(CH₃)—, and substituted or unsubstituted —CH(phenyl)-; L⁵ is —CH₂—; X is selected from the group consisting of substituted and unsubstituted phenyl, substituted or unsubstituted indolyl, and substituted or unsubstituted benzimidazolyl; and Z is selected from the group consisting of methoxymethyl, substituted or unsubstituted phenyl, and substituted or unsubstituted pyrimidyl.

In another embodiment of the compounds of formula IH, M⁴ is —CH₂—; and L⁵ is —C(O)—. In another embodiment of the compounds of formula IH, M⁴ is —N(R⁵)—.

In another embodiment of the compounds of formula IH, M⁴ is —NH—.

In another embodiment of the compounds of formula IH, X is substituted or unsubstituted aryl.

In another embodiment of the compounds of formula IH, X is substituted or unsubstituted phenyl.

In another embodiment of the compounds of formula IH, X is substituted or unsubstituted heteroaryl.

In another embodiment of the compounds of formula IH, X is substituted or unsubstituted pyridyl.

In another embodiment of the compounds of formula IH, X is substituted or unsubstituted pyrimidyl.

In another embodiment of the compounds of formula IH, X is substituted or unsubstituted benzimidazolyl.

In another embodiment of the compounds of formula IH, X is substituted or unsubstituted purinyl.

In another embodiment of the compounds of formula IH, X is substituted or unsubstituted indolyl.

In another embodiment of the compounds of formula IH, X is substituted or unsubstituted thienyl.

In another embodiment of the compounds of formula IH, X is substituted or unsubstituted furanyl.

In another embodiment of the compounds of formula IH, X is substituted or unsubstituted pyrrolyl.

In another embodiment of the compounds of formula IH, X is substituted or unsubstituted quinolyl.

In another embodiment of the compounds of formula IH, M⁴ is —NH—, and L² is —C(R⁴)₂—.

In another embodiment of the compounds of formula IH, M⁴ is —NH—, and L² is —CH₂—.

In another embodiment of the compounds of formula IH, M⁴ is —NH—, and L² is —CH(CH₃)—.

In another embodiment of the compounds of formula IH, M⁴ is —NH—, and L² is —S(O)₂—.

In another embodiment of the compounds of formula IH, M⁴ is —NH—, and L² is —C(O)—.

In another embodiment of the compounds of formula IH, M⁴ is —N(alkyl)-.

In another embodiment of the compounds of formula IH, M⁴ is —N(alkyl)-, and L² is —C(R⁴)₂—.

In another embodiment of the compounds of formula IH, M⁴ is —N(alkyl)-, and L² is —CH₂—.

In another embodiment of the compounds of formula IH, M⁴ is —N(alkyl)-, and L² is —CH(CH₃)—.

In another embodiment of the compounds of formula IH, M⁴ is —N(alkyl)-, and L² is —S(O)₂—.

In another embodiment of the compounds of formula IH, M⁴ is —N(alkyl)-, and L² is —C(O)—.

In another embodiment of the compounds of formula IH, M⁴ is substituted or unsubstituted —N(arylalkyl)-.

In another embodiment of the compounds of formula IH, M⁴ is substituted or unsubstituted —N(arylalkyl)-, and L² is —C(R⁴)₂—.

In another embodiment of the compounds of formula IH, M⁴ is substituted or unsubstituted —N(arylalkyl)-, and L² is —CH₂—.

In another embodiment of the compounds of formula IH, M⁴ is substituted or unsubstituted —N(arylalkyl)-, and L² is —CH(CH₃)—.

In another embodiment of the compounds of formula IH, M⁴ is substituted or unsubstituted —N(arylalkyl)-, and L² is —S(O)₂—.

In another embodiment of the compounds of formula IH, M⁴ is substituted or unsubstituted —N(arylalkyl)-, and L² is —C(O)—.

In another embodiment of the compounds of formula IH, M⁴ is substituted or unsubstituted —N(benzyl)-.

In another embodiment of the compounds of formula IH, M⁴ is substituted or unsubstituted —N(benzyl)-, and L² is —C(R⁴)₂—.

In another embodiment of the compounds of formula IH, M⁴ is substituted or unsubstituted —N(benzyl)-, and L² is —CH₂—.

In another embodiment of the compounds of formula IH, M⁴ is substituted or unsubstituted —N(benzyl)-, and L² is —CH(CH₃)—.

In another embodiment of the compounds of formula IH, M⁴ is substituted or unsubstituted —N(benzyl)-, and L² is —S(O)₂—.

In another embodiment of the compounds of formula IH, M⁴ is substituted or unsubstituted —N(benzyl)-, and L² is —C(O)—.

In another embodiment of the compounds of formula IH, M⁴ is —NH—, L² is —C(R⁴)₂—, and X is substituted or unsubstituted aryl.

In another embodiment of the compounds of formula IH, M⁴ is —NH—, L² is —CH₂—, and X is substituted or unsubstituted aryl.

In another embodiment of the compounds of formula IH, M⁴ is —NH—, L² is —CH(CH₃)—, and X is substituted or unsubstituted aryl.

In another embodiment of the compounds of formula IH, M⁴ is —NH—, L² is —S(O)₂—, and X is substituted or unsubstituted aryl.

In another embodiment of the compounds of formula IH, M⁴ is —NH—, L² is —C(O)—, and X is substituted or unsubstituted aryl.

In another embodiment of the compounds of formula IH, M⁴ is —N(alkyl)-, and X is substituted or unsubstituted aryl.

In another embodiment of the compounds of formula IH, M⁴ is —N(alkyl)-, L² is —C(R⁴)₂—, and X is substituted or unsubstituted aryl.

In another embodiment of the compounds of formula IH, M⁴ is —N(alkyl)-, L² is —CH₂—, and X is substituted or unsubstituted aryl.

In another embodiment of the compounds of formula IH, M⁴ is —N(alkyl)-, L² is —CH(CH₃)—, and X is substituted or unsubstituted aryl.

In another embodiment of the compounds of formula IH, M⁴ is —N(alkyl)-, L² is —S(O)₂—, and X is substituted or unsubstituted aryl.

In another embodiment of the compounds of formula IH, M⁴ is —N(alkyl)-, L² is —C(O)—, and X is substituted or unsubstituted aryl.

In another embodiment of the compounds of formula IH, M⁴ is substituted or unsubstituted —N(arylalkyl)-, and X is substituted or unsubstituted aryl.

In another embodiment of the compounds of formula IH, M⁴ is substituted or unsubstituted —N(arylalkyl)-, L² is —C(R⁴)₂—, and X is substituted or unsubstituted aryl.

In another embodiment of the compounds of formula IH, M⁴ is substituted or unsubstituted —N(arylalkyl)-, L² is —CH₂—, and X is substituted or unsubstituted aryl.

In another embodiment of the compounds of formula IH, M⁴ is substituted or unsubstituted —N(arylalkyl)-, L² is —CH(CH₃)—, and X is substituted or unsubstituted aryl.

In another embodiment of the compounds of formula IH, M⁴ is substituted or unsubstituted —N(arylalkyl)-, L² is —S(O)₂—, and X is substituted or unsubstituted aryl.

In another embodiment of the compounds of formula IH, M⁴ is substituted or unsubstituted —N(arylalkyl)-, L² is —C(O)—, and X is substituted or unsubstituted aryl.

In another embodiment of the compounds of formula IH, M⁴ is substituted or unsubstituted —N(benzyl)-, and X is substituted or unsubstituted aryl.

In another embodiment of the compounds of formula IH, M⁴ is substituted or unsubstituted —N(benzyl)-, L² is —C(R⁴)₂—, and X is substituted or unsubstituted aryl.

In another embodiment of the compounds of formula IH, M⁴ is substituted or unsubstituted —N(benzyl)-, L² is —CH₂—, and X is substituted or unsubstituted aryl.

In another embodiment of the compounds of formula IH, M⁴ is substituted or unsubstituted —N(benzyl)-, L² is —CH(CH₃)—, and X is substituted or unsubstituted aryl.

In another embodiment of the compounds of formula IH, M⁴ is substituted or unsubstituted —N(benzyl)-, L² is —S(O)₂—, and X is substituted or unsubstituted aryl.

In another embodiment of the compounds of formula IH, M⁴ is substituted or unsubstituted —N(benzyl)-, L² is —C(O)—, and X is substituted or unsubstituted aryl.

In another embodiment of the compounds of formula IH, M⁴ is —NH—, L² is —C(R⁴)₂—, and X is substituted or unsubstituted phenyl.

In another embodiment of the compounds of formula IH, M⁴ is —NH—, L² is —CH₂—, and X is substituted or unsubstituted phenyl.

In another embodiment of the compounds of formula IH, M⁴ is —NH—, L² is —CH(CH₃)—, and X is substituted or unsubstituted phenyl.

In another embodiment of the compounds of formula IH, M⁴ is —NH—, L² is —S(O)₂—, and X is substituted or unsubstituted phenyl.

In another embodiment of the compounds of formula IH, M⁴ is —NH—, L² is —C(O)—, and X is substituted or unsubstituted phenyl.

In another embodiment of the compounds of formula IH, M⁴ is —N(alkyl)-, and X is substituted or unsubstituted phenyl.

In another embodiment of the compounds of formula IH, M⁴ is —N(alkyl)-, L² is —C(R⁴)₂—, and X is substituted or unsubstituted phenyl.

In another embodiment of the compounds of formula IH, M⁴ is —N(alkyl)-, L² is —CH₂—, and X is substituted or unsubstituted phenyl.

In another embodiment of the compounds of formula IH, M⁴ is —N(alkyl)-, L² is —CH(CH₃)—, and X is substituted or unsubstituted phenyl.

In another embodiment of the compounds of formula IH, M⁴ is —N(alkyl)-, L² is —S(O)₂—, and X is substituted or unsubstituted phenyl.

In another embodiment of the compounds of formula IH, M⁴ is —N(alkyl)-, L² is —C(O)—, and X is substituted or unsubstituted phenyl.

In another embodiment of the compounds of formula IH, M⁴ is substituted or unsubstituted —N(arylalkyl)-, and X is substituted or unsubstituted phenyl.

In another embodiment of the compounds of formula IH, M⁴ is substituted or unsubstituted —N(arylalkyl)-, L² is —C(R⁴)₂—, and X is substituted or unsubstituted phenyl.

In another embodiment of the compounds of formula IH, M⁴ is substituted or unsubstituted —N(arylalkyl)-, L² is —CH₂—, and X is substituted or unsubstituted phenyl.

In another embodiment of the compounds of formula IH, M⁴ is substituted or unsubstituted —N(arylalkyl)-, L² is —CH(CH₃)—, and X is substituted or unsubstituted phenyl.

In another embodiment of the compounds of formula IH, M⁴ is substituted or unsubstituted —N(arylalkyl)-, L² is —S(O)₂—, and X is substituted or unsubstituted phenyl.

In another embodiment of the compounds of formula IH, M⁴ is substituted or unsubstituted —N(arylalkyl)-, L² is —C(O)—, and X is substituted or unsubstituted phenyl.

In another embodiment of the compounds of formula IH, M⁴ is substituted or unsubstituted —N(benzyl)-, and X is substituted or unsubstituted phenyl.

In another embodiment of the compounds of formula IH, M⁴ is substituted or unsubstituted —N(benzyl)-, L² is —C(R⁴)₂—, and X is substituted or unsubstituted phenyl.

In another embodiment of the compounds of formula IH, M⁴ is substituted or unsubstituted —N(benzyl)-, L² is —CH₂—, and X is substituted or unsubstituted phenyl.

In another embodiment of the compounds of formula IH, M⁴ is substituted or unsubstituted —N(benzyl)-, L² is —CH(CH₃)—, and X is substituted or unsubstituted phenyl.

In another embodiment of the compounds of formula IH, M⁴ is substituted or unsubstituted —N(benzyl)-, L² is —S(O)₂—, and X is substituted or unsubstituted phenyl.

In another embodiment of the compounds of formula IH, M⁴ is substituted or unsubstituted —N(benzyl)-, L² is —C(O)—, and X is substituted or unsubstituted phenyl.

In another embodiment of the compounds of formula IH, M⁴ is —NH—, L² is —C(R⁴)₂—, and X is substituted or unsubstituted heteroaryl.

In another embodiment of the compounds of formula IH, M⁴ is —NH—, L² is —CH₂—, and X is substituted or unsubstituted heteroaryl.

In another embodiment of the compounds of formula IH, M⁴ is —NH—, L¹ is —CH(CH₃)—, and X is substituted or unsubstituted heteroaryl.

In another embodiment of the compounds of formula IH, M⁴ is —NH—, L² is —S(O)₂—, and X is substituted or unsubstituted heteroaryl.

In another embodiment of the compounds of formula IH, M⁴ is —NH—, L² is —C(O)—, and X is substituted or unsubstituted heteroaryl.

In another embodiment of the compounds of formula IH, M⁴ is —N(alkyl)-, and X is substituted or unsubstituted heteroaryl.

In another embodiment of the compounds of formula IH, M⁴ is —N(alkyl)-, L² is —C(R⁴)₂—, and X is substituted or unsubstituted heteroaryl.

In another embodiment of the compounds of formula IH, M⁴ is —N(alkyl)-, L² is —CH₂—, and X is substituted or unsubstituted heteroaryl.

In another embodiment of the compounds of formula IH, M⁴ is —N(alkyl)-, L² is —CH(CH₃)—, and X is substituted or unsubstituted heteroaryl.

In another embodiment of the compounds of formula IH, M⁴ is —N(alkyl)-, L² is —S(O)₂—, and X is substituted or unsubstituted heteroaryl.

In another embodiment of the compounds of formula IH, M⁴ is —N(alkyl)-, L² is —C(O)—, and X is substituted or unsubstituted heteroaryl.

In another embodiment of the compounds of formula IH, M⁴ is substituted or unsubstituted —N(arylalkyl)-, and X is substituted or unsubstituted heteroaryl.

In another embodiment of the compounds of formula IH, M⁴ is substituted or unsubstituted —N(arylalkyl)-, L² is —C(R⁴)₂—, and X is substituted or unsubstituted heteroaryl.

In another embodiment of the compounds of formula IH, M⁴ is substituted or unsubstituted —N(arylalkyl)-, L² is —CH₂—, and X is substituted or unsubstituted heteroaryl.

In another embodiment of the compounds of formula IH, M⁴ is substituted or unsubstituted —N(arylalkyl)-, L² is —CH(CH₃)—, and X is substituted or unsubstituted heteroaryl.

In another embodiment of the compounds of formula IH, M⁴ is substituted or unsubstituted —N(arylalkyl)-, L² is —S(O)₂—, and X is substituted or unsubstituted heteroaryl.

In another embodiment of the compounds of formula IH, M⁴ is substituted or unsubstituted —N(arylalkyl)-, L² is —C(O)—, and X is substituted or unsubstituted heteroaryl.

In another embodiment of the compounds of formula IH, M⁴ is substituted or unsubstituted —N(benzyl)-, and X is substituted or unsubstituted heteroaryl.

In another embodiment of the compounds of formula IH, M⁴ is substituted or unsubstituted —N(benzyl)-, L² is —C(R⁴)₂—, and X is substituted or unsubstituted heteroaryl.

In another embodiment of the compounds of formula IH, M⁴ is substituted or unsubstituted —N(benzyl)-, L² is —CH₂—, and X is substituted or unsubstituted heteroaryl.

In another embodiment of the compounds of formula IH, M⁴ is substituted or unsubstituted —N(benzyl)-, L² is —CH(CH₃)—, and X is substituted or unsubstituted heteroaryl.

In another embodiment of the compounds of formula IH, M⁴ is substituted or unsubstituted —N(benzyl)-, L² is —S(O)₂—, and X is substituted or unsubstituted heteroaryl.

In another embodiment of the compounds of formula IH, M⁴ is substituted or unsubstituted —N(benzyl)-, L² is —C(O)—, and X is substituted or unsubstituted heteroaryl.

In another embodiment of the compounds of formula IH, M⁴ is —NH—, L² is —C(R⁴)₂—, and X is substituted or unsubstituted pyridyl.

In another embodiment of the compounds of formula IH, M⁴ is —NH—, L¹ is —CH₂—, and X is substituted or unsubstituted pyridyl.

In another embodiment of the compounds of formula IH, M⁴ is —NH—, L² is —CH(CH₃)—, and X is substituted or unsubstituted pyridyl.

In another embodiment of the compounds of formula IH, M⁴ is —NH—, L² is —S(O)₂—, and X is substituted or unsubstituted pyridyl.

In another embodiment of the compounds of formula IH, M⁴ is —NH—, L² is —C(O)—, and X is substituted or unsubstituted pyridyl.

In another embodiment of the compounds of formula IH, M⁴ is —N(alkyl)-, and X is substituted or unsubstituted pyridyl.

In another embodiment of the compounds of formula IH, M⁴ is —N(alkyl)-, L² is —C(R⁴)₂—, and X is substituted or unsubstituted pyridyl.

In another embodiment of the compounds of formula IH, M⁴ is —N(alkyl)-, L² is —CH₂—, and X is substituted or unsubstituted pyridyl.

In another embodiment of the compounds of formula IH, M⁴ is —N(alkyl)-, L² is —CH(CH₃)—, and X is substituted or unsubstituted pyridyl.

In another embodiment of the compounds of formula IH, M⁴ is —N(alkyl)-, L² is —S(O)₂—, and X is substituted or unsubstituted pyridyl.

In another embodiment of the compounds of formula IH, M⁴ is —N(alkyl)-, L² is —C(O)—, and X is substituted or unsubstituted pyridyl.

In another embodiment of the compounds of formula IH, M⁴ is substituted or unsubstituted —N(arylalkyl)-, and X is substituted or unsubstituted pyridyl.

In another embodiment of the compounds of formula IH, M⁴ is substituted or unsubstituted —N(arylalkyl)-, L² is —C(R⁴)₂—, and X is substituted or unsubstituted pyridyl.

In another embodiment of the compounds of formula IH, M⁴ is substituted or unsubstituted —N(arylalkyl)-, L² is —CH₂—, and X is substituted or unsubstituted pyridyl.

In another embodiment of the compounds of formula IH, M⁴ is substituted or unsubstituted —N(arylalkyl)-, L² is —CH(CH₃)—, and X is substituted or unsubstituted pyridyl.

In another embodiment of the compounds of formula IH, M⁴ is substituted or unsubstituted —N(arylalkyl)-, L² is —S(O)₂—, and X is substituted or unsubstituted pyridyl.

In another embodiment of the compounds of formula IH, M⁴ is substituted or unsubstituted —N(arylalkyl)-, L² is —C(O)—, and X is substituted or unsubstituted pyridyl.

In another embodiment of the compounds of formula IH, M⁴ is substituted or unsubstituted —N(benzyl)-, and X is substituted or unsubstituted pyridyl.

In another embodiment of the compounds of formula IH, M⁴ is substituted or unsubstituted —N(benzyl)-, L² is —C(R⁴)₂—, and X is substituted or unsubstituted pyridyl.

In another embodiment of the compounds of formula IH, M⁴ is substituted or unsubstituted —N(benzyl)-, L² is —CH₂—, and X is substituted or unsubstituted pyridyl.

In another embodiment of the compounds of formula IH, M⁴ is substituted or unsubstituted —N(benzyl)-, L² is —CH(CH₃)—, and X is substituted or unsubstituted pyridyl.

In another embodiment of the compounds of formula IH, M⁴ is substituted or unsubstituted —N(benzyl)-, L² is —S(O)₂—, and X is substituted or unsubstituted pyridyl.

In another embodiment of the compounds of formula IH, M⁴ is substituted or unsubstituted —N(benzyl)-, L² is —C(O)—, and X is substituted or unsubstituted pyridyl.

In another embodiment of the compounds of formula IH, M⁴ is —NH—, L² is —C(R⁴)₂—, and X is substituted or unsubstituted pyrimidyl.

In another embodiment of the compounds of formula IH, M⁴ is —NH—, L² is —CH₂—, and X is substituted or unsubstituted pyrimidyl.

In another embodiment of the compounds of formula IH, M⁴ is —NH—, L² is —CH(CH₃)—, and X is substituted or unsubstituted pyrimidyl.

In another embodiment of the compounds of formula IH, M⁴ is —NH—, L² is —S(O)₂—, and X is substituted or unsubstituted pyrimidyl.

In another embodiment of the compounds of formula IH, M⁴ is —NH—, L² is —C(O)—, and X is substituted or unsubstituted pyrimidyl.

In another embodiment of the compounds of formula IH, M⁴ is —N(alkyl)-, and X is substituted or unsubstituted pyrimidyl.

In another embodiment of the compounds of formula IH, M⁴ is —N(alkyl)-, L² is —C(R⁴)₂—, and X is substituted or unsubstituted pyrimidyl.

In another embodiment of the compounds of formula IH, M⁴ is —N(alkyl)-, L² is —CH₂—, and X is substituted or unsubstituted pyrimidyl.

In another embodiment of the compounds of formula IH, M⁴ is —N(alkyl)-, L² is —CH(CH₃)—, and X is substituted or unsubstituted pyrimidyl.

In another embodiment of the compounds of formula IH, M⁴ is —N(alkyl)-, L² is —S(O)₂—, and X is substituted or unsubstituted pyrimidyl.

In another embodiment of the compounds of formula IH, M⁴ is —N(alkyl)-, L² is —C(O)—, and X is substituted or unsubstituted pyrimidyl.

In another embodiment of the compounds of formula IH, M⁴ is substituted or unsubstituted —N(arylalkyl)-, and X is substituted or unsubstituted pyrimidyl.

In another embodiment of the compounds of formula IH, M⁴ is substituted or unsubstituted —N(arylalkyl)-, L² is —C(R⁴)₂—, and X is substituted or unsubstituted pyrimidyl.

In another embodiment of the compounds of formula IH, M⁴ is substituted or unsubstituted —N(arylalkyl)-, L² is —CH₂—, and X is substituted or unsubstituted pyrimidyl.

In another embodiment of the compounds of formula IH, M⁴ is substituted or unsubstituted —N(arylalkyl)-, L² is —CH(CH₃)—, and X is substituted or unsubstituted pyrimidyl.

In another embodiment of the compounds of formula IH, M⁴ is substituted or unsubstituted —N(arylalkyl)-, L² is —S(O)₂—, and X is substituted or unsubstituted pyrimidyl.

In another embodiment of the compounds of formula IH, M⁴ is substituted or unsubstituted —N(arylalkyl)-, L² is —C(O)—, and X is substituted or unsubstituted pyrimidyl.

In another embodiment of the compounds of formula IH, M⁴ is substituted or unsubstituted —N(benzyl)-, and X is substituted or unsubstituted pyrimidyl.

In another embodiment of the compounds of formula IH, M⁴ is substituted or unsubstituted —N(benzyl)-, L² is —C(R⁴)₂—, and X is substituted or unsubstituted pyrimidyl.

In another embodiment of the compounds of formula IH, M⁴ is substituted or unsubstituted —N(benzyl)-, L² is —CH₂—, and X is substituted or unsubstituted pyrimidyl.

In another embodiment of the compounds of formula IH, M⁴ is substituted or unsubstituted —N(benzyl)-, L² is —CH(CH₃)—, and X is substituted or unsubstituted pyrimidyl.

In another embodiment of the compounds of formula IH, M⁴ is substituted or unsubstituted —N(benzyl)-, L² is —S(O)₂—, and X is substituted or unsubstituted pyrimidyl.

In another embodiment of the compounds of formula IH, M⁴ is substituted or unsubstituted —N(benzyl)-, L² is —C(O)—, and X is substituted or unsubstituted pyrimidyl.

In another embodiment of the compounds of formula IH, M⁴ is —NH—, L² is —C(R⁴)₂—, and X is substituted or unsubstituted indolyl.

In another embodiment of the compounds of formula IH, M⁴ is —NH—, L² is —CH₂—, and X is substituted or unsubstituted indolyl.

In another embodiment of the compounds of formula IH, M⁴ is —NH—, L² is —CH(CH₃)—, and X is substituted or unsubstituted indolyl.

In another embodiment of the compounds of formula IH, M⁴ is —NH—, L² is —S(O)₂—, and X is substituted or unsubstituted indolyl.

In another embodiment of the compounds of formula IH, M⁴ is —NH—, L² is —C(O)—, and X is substituted or unsubstituted indolyl.

In another embodiment of the compounds of formula IH, M⁴ is —N(alkyl)-, and X is substituted or unsubstituted indolyl.

In another embodiment of the compounds of formula IH, M⁴ is —N(alkyl)-, L² is —C(R⁴)₂—, and X is substituted or unsubstituted indolyl.

In another embodiment of the compounds of formula IH, M⁴ is —N(alkyl)-, L² is —CH₂—, and X is substituted or unsubstituted indolyl.

In another embodiment of the compounds of formula IH, M⁴ is —N(alkyl)-, L² is —CH(CH₃)—, and X is substituted or unsubstituted indolyl.

In another embodiment of the compounds of formula IH, M⁴ is —N(alkyl)-, L² is —S(O)₂—, and X is substituted or unsubstituted indolyl.

In another embodiment of the compounds of formula IH, M⁴ is —N(alkyl)-, L² is —C(O)—, and X is substituted or unsubstituted indolyl.

In another embodiment of the compounds of formula IH, M⁴ is substituted or unsubstituted —N(arylalkyl)-, and X is substituted or unsubstituted indolyl.

In another embodiment of the compounds of formula IH, M⁴ is substituted or unsubstituted —N(arylalkyl)-, L² is —C(R⁴)₂—, and X is substituted or unsubstituted indolyl.

In another embodiment of the compounds of formula IH, M⁴ is substituted or unsubstituted —N(arylalkyl)-, L² is —CH₂—, and X is substituted or unsubstituted indolyl.

In another embodiment of the compounds of formula IH, M⁴ is substituted or unsubstituted —N(arylalkyl)-, L² is —CH(CH₃)—, and X is substituted or unsubstituted indolyl.

In another embodiment of the compounds of formula IH, M⁴ is substituted or unsubstituted —N(arylalkyl)-, L² is —S(O)₂—, and X is substituted or unsubstituted indolyl.

In another embodiment of the compounds of formula IH, M⁴ is substituted or unsubstituted —N(arylalkyl)-, L² is —C(O)—, and X is substituted or unsubstituted indolyl.

In another embodiment of the compounds of formula IH, M⁴ is substituted or unsubstituted —N(benzyl)-, and X is substituted or unsubstituted indolyl.

In another embodiment of the compounds of formula IH, M⁴ is substituted or unsubstituted —N(benzyl)-, L² is —C(R⁴)₂—, and X is substituted or unsubstituted indolyl.

In another embodiment of the compounds of formula IH, M⁴ is substituted or unsubstituted —N(benzyl)-, L² is —CH₂—, and X is substituted or unsubstituted indolyl.

In another embodiment of the compounds of formula IH, M⁴ is substituted or unsubstituted —N(benzyl)-, L² is —CH(CH₃)—, and X is substituted or unsubstituted indolyl.

In another embodiment of the compounds of formula IH, M⁴ is substituted or unsubstituted —N(benzyl)-, L² is —S(O)₂—, and X is substituted or unsubstituted indolyl.

In another embodiment of the compounds of formula IH, M⁴ is substituted or unsubstituted —N(benzyl)-, L² is —C(O)—, and X is substituted or unsubstituted indolyl.

In another embodiment of the compounds of formula IH, M⁴ is —NH—, L² is —C(R⁴)₂—, and X is substituted or unsubstituted benzimidazolyl.

In another embodiment of the compounds of formula IH, M⁴ is —NH—, L² is —CH₂—, and X is substituted or unsubstituted benzimidazolyl.

In another embodiment of the compounds of formula IH, M⁴ is —NH—, L² is —CH(CH₃)—, and X is substituted or unsubstituted benzimidazolyl.

In another embodiment of the compounds of formula IH, M⁴ is —NH—, L² is —S(O)₂—, and X is substituted or unsubstituted benzimidazolyl.

In another embodiment of the compounds of formula IH, M⁴ is —NH—, L² is —C(O)—, and X is substituted or unsubstituted benzimidazolyl.

In another embodiment of the compounds of formula IH, M⁴ is —N(alkyl)-, and X is substituted or unsubstituted benzimidazolyl.

In another embodiment of the compounds of formula IH, M⁴ is —N(alkyl)-, L² is —C(R⁴)₂—, and X is substituted or unsubstituted benzimidazolyl.

In another embodiment of the compounds of formula IH, M⁴ is —N(alkyl)-, L² is —CH₂—, and X is substituted or unsubstituted benzimidazolyl.

In another embodiment of the compounds of formula IH, M⁴ is —N(alkyl)-, L² is —CH(CH₃)—, and X is substituted or unsubstituted benzimidazolyl.

In another embodiment of the compounds of formula IH, M⁴ is —N(alkyl)-, L² is —S(O)₂—, and X is substituted or unsubstituted benzimidazolyl.

In another embodiment of the compounds of formula IH, M⁴ is —N(alkyl)-, L² is —C(O)—, and X is substituted or unsubstituted benzimidazolyl.

In another embodiment of the compounds of formula IH, M⁴ is substituted or unsubstituted —N(arylalkyl)-, and X is substituted or unsubstituted benzimidazolyl.

In another embodiment of the compounds of formula IH, M⁴ is substituted or unsubstituted —N(arylalkyl)-, L² is —C(R⁴)₂—, and X is substituted or unsubstituted benzimidazolyl.

In another embodiment of the compounds of formula IH, M⁴ is substituted or unsubstituted —N(arylalkyl)-, L² is —CH₂—, and X is substituted or unsubstituted benzimidazolyl.

In another embodiment of the compounds of formula IH, M⁴ is substituted or unsubstituted —N(arylalkyl)-, L² is —CH(CH₃)—, and X is substituted or unsubstituted benzimidazolyl.

In another embodiment of the compounds of formula IH, M⁴ is substituted or unsubstituted —N(arylalkyl)-, L² is —S(O)₂—, and X is substituted or unsubstituted benzimidazolyl.

In another embodiment of the compounds of formula IH, M⁴ is substituted or unsubstituted —N(arylalkyl)-, L² is —C(O)—, and X is substituted or unsubstituted benzimidazolyl.

In another embodiment of the compounds of formula IH, M⁴ is substituted or unsubstituted —N(benzyl)-, and X is substituted or unsubstituted benzimidazolyl.

In another embodiment of the compounds of formula IH, M⁴ is substituted or unsubstituted —N(benzyl)-, L² is —C(R⁴)₂—, and X is substituted or unsubstituted benzimidazolyl.

In another embodiment of the compounds of formula IH, M⁴ is substituted or unsubstituted —N(benzyl)-, L² is —CH₂—, and X is substituted or unsubstituted benzimidazolyl.

In another embodiment of the compounds of formula IH, M⁴ is substituted or unsubstituted —N(benzyl)-, L² is —CH(CH₃)—, and X is substituted or unsubstituted benzimidazolyl.

In another embodiment of the compounds of formula IH, M⁴ is substituted or unsubstituted —N(benzyl)-, L² is —S(O)₂—, and X is substituted or unsubstituted benzimidazolyl.

In another embodiment of the compounds of formula IH, M⁴ is substituted or unsubstituted —N(benzyl)-, L² is —C(O)—, and X is substituted or unsubstituted benzimidazolyl.

In another embodiment of the compounds of formula IH, M⁴ is —NH—, L² is —C(R⁴)₂—, and X is substituted or unsubstituted quinolyl.

In another embodiment of the compounds of formula IH, M⁴ is —NH—, L² is —CH₂—, and X is substituted or unsubstituted quinolyl.

In another embodiment of the compounds of formula IH, M⁴ is —NH—, L² is —CH(CH₃)—, and X is substituted or unsubstituted quinolyl.

In another embodiment of the compounds of formula IH, M⁴ is —NH—, L² is —S(O)₂—, and X is substituted or unsubstituted quinolyl.

In another embodiment of the compounds of formula IH, M⁴ is —NH—, L² is —C(O)—, and X is substituted or unsubstituted quinolyl.

In another embodiment of the compounds of formula IH, M⁴ is —N(alkyl)-, and X is substituted or unsubstituted quinolyl.

In another embodiment of the compounds of formula IH, M⁴ is —N(alkyl)-, L² is —C(R⁴)₂—, and X is substituted or unsubstituted quinolyl.

In another embodiment of the compounds of formula IH, M⁴ is —N(alkyl)-, L² is —CH₂—, and X is substituted or unsubstituted quinolyl.

In another embodiment of the compounds of formula IH, M⁴ is —N(alkyl)-, L² is —CH(CH₃)—, and X is substituted or unsubstituted quinolyl.

In another embodiment of the compounds of formula IH, M⁴ is —N(alkyl)-, L² is —S(O)₂—, and X is substituted or unsubstituted quinolyl.

In another embodiment of the compounds of formula IH, M⁴ is —N(alkyl)-, L² is —C(O)—, and X is substituted or unsubstituted quinolyl.

In another embodiment of the compounds of formula IH, M⁴ is substituted or unsubstituted —N(arylalkyl)-, and X is substituted or unsubstituted quinolyl.

In another embodiment of the compounds of formula IH, M⁴ is substituted or unsubstituted —N(arylalkyl)-, L² is —C(R⁴)₂—, and X is substituted or unsubstituted quinolyl.

In another embodiment of the compounds of formula IH, M⁴ is substituted or unsubstituted —N(arylalkyl)-, L² is —CH₂—, and X is substituted or unsubstituted quinolyl.

In another embodiment of the compounds of formula IH, M⁴ is substituted or unsubstituted —N(arylalkyl)-, L² is —CH(CH₃)—, and X is substituted or unsubstituted quinolyl.

In another embodiment of the compounds of formula IH, M⁴ is substituted or unsubstituted —N(arylalkyl)-, L² is —S(O)₂—, and X is substituted or unsubstituted quinolyl.

In another embodiment of the compounds of formula IH, M⁴ is substituted or unsubstituted —N(arylalkyl)-, L² is —C(O)—, and X is substituted or unsubstituted quinolyl.

In another embodiment of the compounds of formula IH, M⁴ is substituted or unsubstituted —N(benzyl)-, and X is substituted or unsubstituted quinolyl.

In another embodiment of the compounds of formula IH, M⁴ is substituted or unsubstituted —N(benzyl)-, L² is —C(R⁴)₂—, and X is substituted or unsubstituted quinolyl.

In another embodiment of the compounds of formula IH, M⁴ is substituted or unsubstituted —N(benzyl)-, L² is —CH₂—, and X is substituted or unsubstituted quinolyl.

In another embodiment of the compounds of formula IH, M⁴ is substituted or unsubstituted —N(benzyl)-, L² is —CH(CH₃)—, and X is substituted or unsubstituted quinolyl.

In another embodiment of the compounds of formula IH, M⁴ is substituted or unsubstituted —N(benzyl)-, L² is —S(O)₂—, and X is substituted or unsubstituted quinolyl.

In another embodiment of the compounds of formula IH, M⁴ is substituted or unsubstituted —N(benzyl)-, L² is —C(O)—, and X is substituted or unsubstituted quinolyl.

In another embodiment of the compounds of formula IH, M⁴ is —NH—, L² is —C(R⁴)₂—, and X is substituted or unsubstituted purinyl.

In another embodiment of the compounds of formula IH, M⁴ is —NH—, L² is —CH₂—, and X is substituted or unsubstituted purinyl.

In another embodiment of the compounds of formula IH, M⁴ is —NH—, L² is —CH(CH₃)—, and X is substituted or unsubstituted purinyl.

In another embodiment of the compounds of formula IH, M⁴ is —NH—, L² is —S(O)₂—, and X is substituted or unsubstituted purinyl.

In another embodiment of the compounds of formula IH, M⁴ is —NH—, L² is —C(O)—, and X is substituted or unsubstituted purinyl.

In another embodiment of the compounds of formula IH, M⁴ is —N(alkyl)-, and X is substituted or unsubstituted purinyl.

In another embodiment of the compounds of formula IH, M⁴ is —N(alkyl)-, L² is —C(R⁴)₂—, and X is substituted or unsubstituted purinyl.

In another embodiment of the compounds of formula IH, M⁴ is —N(alkyl)-, L² is —CH₂—, and X is substituted or unsubstituted purinyl.

In another embodiment of the compounds of formula IH, M⁴ is —N(alkyl)-, L² is —CH(CH₃)—, and X is substituted or unsubstituted purinyl.

In another embodiment of the compounds of formula IH, M is —N(alkyl)-, L² is —S(O)₂—, and X is substituted or unsubstituted purinyl.

In another embodiment of the compounds of formula IH, M⁴ is —N(alkyl)-, L² is —C(O)—, and X is substituted or unsubstituted purinyl.

In another embodiment of the compounds of formula IH, M⁴ is substituted or unsubstituted —N(arylalkyl)-, and X is substituted or unsubstituted purinyl.

In another embodiment of the compounds of formula IH, M⁴ is substituted or unsubstituted —N(arylalkyl)-, L² is —C(R⁴)₂—, and X is substituted or unsubstituted purinyl.

In another embodiment of the compounds of formula IH, M⁴ is substituted or unsubstituted —N(arylalkyl)-, L² is —CH₂—, and X is substituted or unsubstituted purinyl.

In another embodiment of the compounds of formula IH, M⁴ is substituted or unsubstituted —N(arylalkyl)-, L² is —CH(CH₃)—, and X is substituted or unsubstituted purinyl.

In another embodiment of the compounds of formula IH, M⁴ is substituted or unsubstituted —N(arylalkyl)-, L² is —S(O)₂—, and X is substituted or unsubstituted purinyl.

In another embodiment of the compounds of formula IH, M⁴ is substituted or unsubstituted —N(arylalkyl)-, L² is —C(O)—, and X is substituted or unsubstituted purinyl.

In another embodiment of the compounds of formula IH, M⁴ is substituted or unsubstituted —N(benzyl)-, and X is substituted or unsubstituted purinyl.

In another embodiment of the compounds of formula IH, M⁴ is substituted or unsubstituted —N(benzyl)-, L² is —C(R⁴)₂—, and X is substituted or unsubstituted purinyl.

In another embodiment of the compounds of formula IH, M⁴ is substituted or unsubstituted —N(benzyl)-, L² is —CH₂—, and X is substituted or unsubstituted purinyl.

In another embodiment of the compounds of formula IH, M⁴ is substituted or unsubstituted —N(benzyl)-, L² is —CH(CH₃)—, and X is substituted or unsubstituted purinyl.

In another embodiment of the compounds of formula IH, M⁴ is substituted or unsubstituted —N(benzyl)-, L² is —S(O)₂—, and X is substituted or unsubstituted purinyl.

In another embodiment of the compounds of formula IH, M⁴ is substituted or unsubstituted —N(benzyl)-, L² is —C(O)—, and X is substituted or unsubstituted purinyl.

In another embodiment of the compounds of formula IH, M⁴ is —NH—, L² is —C(R⁴)₂—, and X is substituted or unsubstituted thienyl.

In another embodiment of the compounds of formula IH, M⁴ is —NH—, L² is —CH₂—, and X is substituted or unsubstituted thienyl.

In another embodiment of the compounds of formula IH, M⁴ is —NH—, L² is —CH(CH₃)—, and X is substituted or unsubstituted thienyl.

In another embodiment of the compounds of formula IH, M⁴ is —NH—, L² is —S(O)₂—, and X is substituted or unsubstituted thienyl.

In another embodiment of the compounds of formula IH, M⁴ is —NH—, L² is —C(O)—, and X is substituted or unsubstituted thienyl.

In another embodiment of the compounds of formula IH, M⁴ is —N(alkyl)-, and X is substituted or unsubstituted thienyl.

In another embodiment of the compounds of formula IH, M⁴ is —N(alkyl)-, L² is —C(R⁴)₂—, and X is substituted or unsubstituted thienyl.

In another embodiment of the compounds of formula IH, M⁴ is —N(alkyl)-, L² is —CH₂—, and X is substituted or unsubstituted thienyl.

In another embodiment of the compounds of formula IH, M⁴ is —N(alkyl)-, L² is —CH(CH₃)—, and X is substituted or unsubstituted thienyl.

In another embodiment of the compounds of formula IH, M⁴ is —N(alkyl)-, L² is —S(O)₂—, and X is substituted or unsubstituted thienyl.

In another embodiment of the compounds of formula IH, M⁴ is —N(alkyl)-, L² is —C(O)—, and X is substituted or unsubstituted thienyl.

In another embodiment of the compounds of formula IH, M⁴ is substituted or unsubstituted —N(arylalkyl)-, and X is substituted or unsubstituted thienyl.

In another embodiment of the compounds of formula IH, M⁴ is substituted or unsubstituted —N(arylalkyl)-, L² is —C(R⁴)₂—, and X is substituted or unsubstituted thienyl.

In another embodiment of the compounds of formula IH, M⁴ is substituted or unsubstituted —N(arylalkyl)-, L² is —CH₂—, and X is substituted or unsubstituted thienyl.

In another embodiment of the compounds of formula IH, M⁴ is substituted or unsubstituted —N(arylalkyl)-, L² is —CH(CH₃)—, and X is substituted or unsubstituted thienyl.

In another embodiment of the compounds of formula IH, M⁴ is substituted or unsubstituted —N(arylalkyl)-, L² is —S(O)₂—, and X is substituted or unsubstituted thienyl.

In another embodiment of the compounds of formula IH, M⁴ is substituted or unsubstituted —N(arylalkyl)-, L² is —C(O)—, and X is substituted or unsubstituted thienyl.

In another embodiment of the compounds of formula IH, M⁴ is substituted or unsubstituted —N(benzyl)-, and X is substituted or unsubstituted thienyl.

In another embodiment of the compounds of formula IH, M⁴ is substituted or unsubstituted —N(benzyl)-, L² is —C(R⁴)₂—, and X is substituted or unsubstituted thienyl.

In another embodiment of the compounds of formula IH, M⁴ is substituted or unsubstituted —N(benzyl)-, L² is —CH₂—, and X is substituted or unsubstituted thienyl.

In another embodiment of the compounds of formula IH, M⁴ is substituted or unsubstituted —N(benzyl)-, L² is —CH(CH₃)—, and X is substituted or unsubstituted thienyl.

In another embodiment of the compounds of formula IH, M⁴ is substituted or unsubstituted —N(benzyl)-, L² is —S(O)₂—, and X is substituted or unsubstituted thienyl.

In another embodiment of the compounds of formula IH, M⁴ is substituted or unsubstituted —N(benzyl)-, L² is —C(O)—, and X is substituted or unsubstituted thienyl.

In another embodiment of the compounds of formula IH, M⁴ is —NH—, L² is —C(R⁴)₂—, and X is substituted or unsubstituted furanyl.

In another embodiment of the compounds of formula IH, M⁴ is —NH—, L² is —CH₂—, and X is substituted or unsubstituted furanyl.

In another embodiment of the compounds of formula IH, M⁴ is —NH—, L² is —CH(CH₃)—, and X is substituted or unsubstituted furanyl.

In another embodiment of the compounds of formula IH, M⁴ is —NH—, L² is —S(O)₂—, and X is substituted or unsubstituted furanyl.

In another embodiment of the compounds of formula IH, M⁴ is —NH—, L² is —C(O)—, and X is substituted or unsubstituted furanyl.

In another embodiment of the compounds of formula IH, M⁴ is —N(alkyl)-, and X is substituted or unsubstituted furanyl.

In another embodiment of the compounds of formula IH, M⁴ is —N(alkyl)-, L² is —C(R⁴)₂—, and X is substituted or unsubstituted furanyl.

In another embodiment of the compounds of formula IH, M⁴ is —N(alkyl)-, L² is —CH₂—, and X is substituted or unsubstituted furanyl.

In another embodiment of the compounds of formula IH, M⁴ is —N(alkyl)-, L² is —CH(CH₃)—, and X is substituted or unsubstituted furanyl.

In another embodiment of the compounds of formula IH, M⁴ is —N(alkyl)-, L² is —S(O)₂—, and X is substituted or unsubstituted furanyl.

In another embodiment of the compounds of formula IH, M⁴ is —N(alkyl)-, L² is —C(O)—, and X is substituted or unsubstituted furanyl.

In another embodiment of the compounds of formula IH, M⁴ is substituted or unsubstituted —N(arylalkyl)-, and X is substituted or unsubstituted furanyl.

In another embodiment of the compounds of formula IH, M⁴ is substituted or unsubstituted —N(arylalkyl)-, L² is —C(R⁴)₂—, and X is substituted or unsubstituted furanyl.

In another embodiment of the compounds of formula IH, M⁴ is substituted or unsubstituted —N(arylalkyl)-, L² is —CH₂—, and X is substituted or unsubstituted furanyl.

In another embodiment of the compounds of formula IH, M⁴ is substituted or unsubstituted —N(arylalkyl)-, L² is —CH(CH₃)—, and X is substituted or unsubstituted furanyl.

In another embodiment of the compounds of formula IH, M⁴ is substituted or unsubstituted —N(arylalkyl)-, L² is —S(O)₂—, and X is substituted or unsubstituted furanyl.

In another embodiment of the compounds of formula IH, M⁴ is substituted or unsubstituted —N(arylalkyl)-, L² is —C(O)—, and X is substituted or unsubstituted furanyl.

In another embodiment of the compounds of formula IH, M⁴ is substituted or unsubstituted —N(benzyl)-, and X is substituted or unsubstituted furanyl.

In another embodiment of the compounds of formula IH, M⁴ is substituted or unsubstituted —N(benzyl)-, L² is —C(R⁴)₂—, and X is substituted or unsubstituted furanyl.

In another embodiment of the compounds of formula IH, M⁴ is substituted or unsubstituted —N(benzyl)-, L² is —CH₂—, and X is substituted or unsubstituted furanyl.

In another embodiment of the compounds of formula IH, M⁴ is substituted or unsubstituted —N(benzyl)-, L² is —CH(CH₃)—, and X is substituted or unsubstituted furanyl.

In another embodiment of the compounds of formula IH, M⁴ is substituted or unsubstituted —N(benzyl)-, L² is —S(O)₂—, and X is substituted or unsubstituted furanyl.

In another embodiment of the compounds of formula IH, M⁴ is substituted or unsubstituted —N(benzyl)-, L² is —C(O)—, and X is substituted or unsubstituted furanyl.

In another embodiment of the compounds of formula IH, M⁴ is —NH—, L² is —C(R⁴)₂—, and X is substituted or unsubstituted pyrrolyl.

In another embodiment of the compounds of formula IH, M⁴ is —NH—, L² is —CH₂—, and X is substituted or unsubstituted pyrrolyl.

In another embodiment of the compounds of formula IH, M⁴ is —NH—, L² is —CH(CH₃)—, and X is substituted or unsubstituted pyrrolyl.

In another embodiment of the compounds of formula IH, M⁴ is —NH—, L² is —S(O)₂—, and X is substituted or unsubstituted pyrrolyl.

In another embodiment of the compounds of formula IH, M⁴ is —NH—, L² is —C(O)—, and X is substituted or unsubstituted pyrrolyl.

In another embodiment of the compounds of formula IH, M⁴ is —N(alkyl)-, and X is substituted or unsubstituted pyrrolyl.

In another embodiment of the compounds of formula IH, M⁴ is —N(alkyl)-, L² is —C(R⁴)₂—, and X is substituted or unsubstituted pyrrolyl.

In another embodiment of the compounds of formula IH, M⁴ is —N(alkyl)-, L² is —CH₂—, and X is substituted or unsubstituted pyrrolyl.

In another embodiment of the compounds of formula IH, M⁴ is —N(alkyl)-, L² is —CH(CH₃)—, and X is substituted or unsubstituted pyrrolyl.

In another embodiment of the compounds of formula IH, M⁴ is —N(alkyl)-, L² is —S(O)₂—, and X is substituted or unsubstituted pyrrolyl.

In another embodiment of the compounds of formula IH, M⁴ is —N(alkyl)-, L² is —C(O)—, and X is substituted or unsubstituted pyrrolyl.

In another embodiment of the compounds of formula IH, M⁴ is substituted or unsubstituted —N(arylalkyl)-, and X is substituted or unsubstituted pyrrolyl.

In another embodiment of the compounds of formula IH, M⁴ is substituted or unsubstituted —N(arylalkyl)-, L² is —C(R⁴)₂—, and X is substituted or unsubstituted pyrrolyl.

In another embodiment of the compounds of formula IH, M⁴ is substituted or unsubstituted —N(arylalkyl)-, L² is —CH₂—, and X is substituted or unsubstituted pyrrolyl.

In another embodiment of the compounds of formula IH, M⁴ is substituted or unsubstituted —N(arylalkyl)-, L² is —CH(CH₃)—, and X is substituted or unsubstituted pyrrolyl.

In another embodiment of the compounds of formula IH, M⁴ is substituted or unsubstituted —N(arylalkyl)-, L² is —S(O)₂—, and X is substituted or unsubstituted pyrrolyl.

In another embodiment of the compounds of formula IH, M⁴ is substituted or unsubstituted —N(arylalkyl)-, L² is —C(O)—, and X is substituted or unsubstituted pyrrolyl.

In another embodiment of the compounds of formula IH, M⁴ is substituted or unsubstituted —N(benzyl)-, and X is substituted or unsubstituted pyrrolyl.

In another embodiment of the compounds of formula IH, M⁴ is substituted or unsubstituted —N(benzyl)-, L² is —C(R⁴)₂—, and X is substituted or unsubstituted pyrrolyl.

In another embodiment of the compounds of formula IH, M⁴ is substituted or unsubstituted —N(benzyl)-, L² is —CH₂—, and X is substituted or unsubstituted pyrrolyl.

In another embodiment of the compounds of formula IH, M⁴ is substituted or unsubstituted —N(benzyl)-, L² is —CH(CH₃)—, and X is substituted or unsubstituted pyrrolyl.

In another embodiment of the compounds of formula IH, M⁴ is substituted or unsubstituted —N(benzyl)-, L² is —S(O)₂—, and X is substituted or unsubstituted pyrrolyl.

In another embodiment of the compounds of formula IH, M⁴ is substituted or unsubstituted —N(benzyl)-, L² is —C(O)—, and X is substituted or unsubstituted pyrrolyl.

In another embodiment of the compounds of formula IH, R² is H.

In another embodiment of the compounds of formula IH, R² is alkyl.

In another embodiment of the compounds of formula IH, R² is alkoxy.

In another embodiment of the compounds of formula IH, R² is alkylthio.

In another embodiment of the compounds of formula IH, R² is haloalkyl.

In another embodiment of the compounds of formula IH, M⁴ is —N(R⁵)—, and L⁵ is —C(R⁴)₂—.

In another embodiment of the compounds of formula IH, M⁴ is —N(R⁵)—, and L₅ is —CH₂—.

In another embodiment of the compounds of formula IH, M⁴ is —N(R⁵)—, and L⁵ is —CH(CH₃)—.

In another embodiment of the compounds of formula IH, M⁴ is —N(R⁵)—, and L⁵ is —C(O)—.

In another embodiment of the compounds of formula IH, M⁴ is —N(R⁵)—, and L⁵ is —S(O)₂—.

In another embodiment of the compounds of formula IH, M⁴ is —N(R⁵)—, L⁵ is —C(R⁴)₂—, and Z is substituted or unsubstituted aryl.

In another embodiment of the compounds of formula IH, M⁴ is —N(R⁵)—, L⁵ is —CH₂—, and Z is substituted or unsubstituted aryl.

In another embodiment of the compounds of formula IH, M⁴ is —N(R⁵)—, L⁵ is —CH(CH₃)—, and Z is substituted or unsubstituted aryl.

In another embodiment of the compounds of formula IH, M⁴ is —N(R⁵)—, L⁵ is —C(O)—, and Z is substituted or unsubstituted aryl.

In another embodiment of the compounds of formula IH, M⁴ is —N(R⁵)—, L⁵ is —S(O)₂—, and Z is substituted or unsubstituted aryl.

In another embodiment of the compounds of formula IH, M⁴ is —N(R⁵)—, L⁵ is —C(R⁴)₂—, and Z is substituted or unsubstituted phenyl.

In another embodiment of the compounds of formula IH, M⁴ is —N(R⁵)—, L⁵ is —CH₂—, and Z is substituted or unsubstituted phenyl.

In another embodiment of the compounds of formula IH, M⁴ is —N(R⁵)—, L⁵ is —CH(CH₃)—, and Z is substituted or unsubstituted phenyl.

In another embodiment of the compounds of formula IH, M⁴ is —N(R⁵)—, L⁵ is —C(O)—, and Z is substituted or unsubstituted phenyl.

In another embodiment of the compounds of formula IH, M⁴ is —N(R⁵)—, L⁵ is —S(O)₂—, and Z is substituted or unsubstituted phenyl.

In another embodiment of the compounds of formula IH, M⁴ is —N(R⁵)—, L⁵ is —C(R⁴)₂—, and Z is substituted or unsubstituted heteroaryl.

In another embodiment of the compounds of formula IH, M⁴ is —N(R⁵)—, L⁵ is —CH₂—, and Z is substituted or unsubstituted heteroaryl.

In another embodiment of the compounds of formula IH, M⁴ is —N(R⁵)—, L⁵ is —CH(CH₃)—, and Z is substituted or unsubstituted heteroaryl.

In another embodiment of the compounds of formula IH, M⁴ is —N(R⁵)—, L⁵ is —C(O)—, and Z is substituted or unsubstituted heteroaryl.

In another embodiment of the compounds of formula IH, M⁴ is —N(R⁵)—, L⁵ is —S(O)₂—, and Z is substituted or unsubstituted heteroaryl.

In another embodiment of the compounds of formula IH, M⁴ is —N(R⁵)—, L⁵ is —C(R⁴)₂—, and Z is substituted or unsubstituted pyridyl.

In another embodiment of the compounds of formula IH, M⁴ is —N(R⁵)—, L⁵ is —CH₂—, and Z is substituted or unsubstituted pyridyl.

In another embodiment of the compounds of formula IH, M⁴ is —N(R⁵)—, L⁵ is —CH(CH₃)—, and Z is substituted or unsubstituted pyridyl.

In another embodiment of the compounds of formula IH, M⁴ is —N(R⁵)—, L⁵ is —C(O)—, and Z is substituted or unsubstituted pyridyl.

In another embodiment of the compounds of formula IH, M⁴ is —N(R⁵)—, L⁵ is —S(O)₂—, and Z is substituted or unsubstituted pyridyl.

In another embodiment of the compounds of formula IH, M⁴ is —N(R⁵)—, L⁵ is —C(R⁴)₂—, and Z is substituted or unsubstituted pyrimidyl.

In another embodiment of the compounds of formula IH, M⁴ is —N(R⁵)—, L⁵ is —CH₂—, and Z is substituted or unsubstituted pyrimidyl.

In another embodiment of the compounds of formula IH, M⁴ is —N(R⁵)—, L⁵ is —CH(CH₃)—, and Z is substituted or unsubstituted pyrimidyl.

In another embodiment of the compounds of formula IH, M⁴ is —N(R⁵)—, L⁵ is —C(O)—, and Z is substituted or unsubstituted pyrimidyl.

In another embodiment of the compounds of formula IH, M⁴ is —N(R⁵)—, L⁵ is —S(O)₂—, and Z is substituted or unsubstituted pyrimidyl.

In another embodiment of the compounds of formula IH, M⁴ is —N(R⁵)—, L⁵ is —C(R⁴)₂—, and Z is substituted or unsubstituted indolyl.

In another embodiment of the compounds of formula IH, M⁴ is —N(R⁵)—, L⁵ is —CH₂—, and Z is substituted or unsubstituted indolyl.

In another embodiment of the compounds of formula IH, M⁴ is —N(R⁵)—, L⁵ is —CH(CH₃)—, and Z is substituted or unsubstituted indolyl.

In another embodiment of the compounds of formula IH, M⁴ is —N(R⁵)—, L⁵ is —C(O)—, and Z is substituted or unsubstituted indolyl.

In another embodiment of the compounds of formula IH, M⁴ is —N(R⁵)—, L⁵ is —S(O)₂—, and Z is substituted or unsubstituted indolyl.

In another embodiment of the compounds of formula IH, M⁴ is —N(R⁵)—, L⁵ is —C(R⁴)₂—, and Z is substituted or unsubstituted quinolyl.

In another embodiment of the compounds of formula IH, M⁴ is —N(R⁵)—, L⁵ is —CH₂—, and Z is substituted or unsubstituted quinolyl.

In another embodiment of the compounds of formula IH, M⁴ is —N(R⁵)—, L⁵ is —CH(CH₃)—, and Z is substituted or unsubstituted quinolyl.

In another embodiment of the compounds of formula IH, M⁴ is —N(R⁵)—, L⁵ is —C(O)—, and Z is substituted or unsubstituted quinolyl.

In another embodiment of the compounds of formula IH, M⁴ is —N(R⁵)—, L⁵ is —S(O)₂—, and Z is substituted or unsubstituted quinolyl.

In another embodiment of the compounds of formula IH, M⁴ is —N(R⁵)—, L⁵ is —C(R⁴)₂—, and Z is substituted or unsubstituted benzimidazolyl.

In another embodiment of the compounds of formula IH, M⁴ is —N(R⁵)—, L⁵ is —CH₂—, and Z is substituted or unsubstituted benzimidazolyl.

In another embodiment of the compounds of formula IH, M⁴ is —N(R⁵)—, L⁵ is —CH(CH₃)—, and Z is substituted or unsubstituted benzimidazolyl.

In another embodiment of the compounds of formula IH, M⁴ is —N(R⁵)—, L⁵ is —C(O)—, and Z is substituted or unsubstituted benzimidazolyl.

In another embodiment of the compounds of formula IH, M⁴ is —N(R⁵)—, L⁵ is —S(O)₂—, and Z is substituted or unsubstituted benzimidazolyl.

In another embodiment of the compounds of formula IH, M⁴ is —N(R⁵)—, L⁵ is —C(R⁴)₂—, and Z is substituted or unsubstituted thienyl.

In another embodiment of the compounds of formula IH, M⁴ is —N(R⁵)—, L⁵ is —CH₂—, and Z is substituted or unsubstituted thienyl.

In another embodiment of the compounds of formula IH, M⁴ is —N(R⁵)—, L⁵ is —CH(CH₃)—, and Z is substituted or unsubstituted thienyl.

In another embodiment of the compounds of formula IH, M⁴ is —N(R⁵)—, L⁵ is —C(O)—, and Z is substituted or unsubstituted thienyl.

In another embodiment of the compounds of formula IH, M⁴ is —N(R⁵)—, L⁵ is —S(O)₂—, and Z is substituted or unsubstituted thienyl.

In another embodiment of the compounds of formula IH, M⁴ is —N(R⁵)—, L⁵ is —C(R⁴)₂—, and Z is substituted or unsubstituted furanyl.

In another embodiment of the compounds of formula IH, M⁴ is —N(R⁵)—, L⁵ is —CH₂—, and Z is substituted or unsubstituted furanyl.

In another embodiment of the compounds of formula IH, M⁴ is —N(R⁵)—, L⁵ is —CH(CH₃)—, and Z is substituted or unsubstituted furanyl.

In another embodiment of the compounds of formula IH, M⁴ is —N(R⁵)—, L⁵ is —C(O)—, and Z is substituted or unsubstituted furanyl.

In another embodiment of the compounds of formula IH, M⁴ is —N(R⁵)—, L⁵ is —S(O)₂—, and Z is substituted or unsubstituted furanyl.

In another embodiment of the compounds of formula IH, M⁴ is —N(R⁵)—, L⁵ is —C(R⁴)₂—, and Z is substituted or unsubstituted pyrrolyl.

In another embodiment of the compounds of formula IH, M⁴ is —N(R⁵)—, L⁵ is —CH₂—, and Z is substituted or unsubstituted pyrrolyl.

In another embodiment of the compounds of formula IH, M⁴ is —N(R⁵)—, L⁵ is —CH(CH₃)—, and Z is substituted or unsubstituted pyrrolyl.

In another embodiment of the compounds of formula IH, M⁴ is —N(R⁵)—, L⁵ is —C(O)—, and Z is substituted or unsubstituted pyrrolyl.

In another embodiment of the compounds of formula IH, M⁴ is —N(R⁵)—, L⁵ is —S(O)₂—, and Z is substituted or unsubstituted pyrrolyl.

In another embodiment of the compounds of formula IH, M⁴ is —N(R⁵)—, L⁵ is —C(R⁴)₂—, and Z is substituted or unsubstituted purinyl.

In another embodiment of the compounds of formula IH, M⁴ is —N(R⁵)—, L⁵ is —CH₂—, and Z is substituted or unsubstituted purinyl.

In another embodiment of the compounds of formula IH, M⁴ is —N(R⁵)—, L⁵ is —CH(CH₃)—, and Z is substituted or unsubstituted purinyl.

In another embodiment of the compounds of formula IH, M⁴ is —N(R⁵)—, L⁵ is —C(O)—, and Z is substituted or unsubstituted purinyl.

In another embodiment of the compounds of formula IH, M⁴ is —N(R⁵)—, L⁵ is —S(O)₂—, and Z is substituted or unsubstituted purinyl.

In another embodiment of the compounds of formula IH, M⁴ is —N(R⁵)—, L⁵ is —C(R⁴)₂—, and Z is substituted or unsubstituted alkoxyalkyl.

In another embodiment of the compounds of formula IH, M⁴ is —N(R⁵)—, L⁵ is —CH₂—, and Z is substituted or unsubstituted alkoxyalkyl.

In another embodiment of the compounds of formula IH, M⁴ is —N(R⁵)—, L⁵ is —CH(CH₃)—, and Z is substituted or unsubstituted alkoxyalkyl.

In another embodiment of the compounds of formula IH, M⁴ is —N(R⁵)—, L⁵ is —C(O)—, and Z is substituted or unsubstituted alkoxyalkyl.

In another embodiment of the compounds of formula IH, M⁴ is —N(R⁵)—, L⁵ is —S(O)₂—, and Z is substituted or unsubstituted alkoxyalkyl.

In another embodiment of the compounds of formula IH, M⁴ is —N(R⁵)—, L⁵ is —C(R⁴)₂—, and Z is substituted or unsubstituted methoxyalkyl.

In another embodiment of the compounds of formula IH, M⁴ is —N(R⁵)—, L⁵ is —CH₂—, and Z is substituted or unsubstituted methoxyalkyl.

In another embodiment of the compounds of formula IH, M⁴ is —N(R⁵)—, L⁵ is —CH(CH₃)—, and Z is substituted or unsubstituted methoxyalkyl.

In another embodiment of the compounds of formula IH, M⁴ is —N(R⁵)—, L⁵ is —C(O)—, and Z is substituted or unsubstituted methoxyalkyl.

In another embodiment of the compounds of formula IH, M⁴ is —N(R⁵)—, L⁵ is —S(O)₂—, and Z is substituted or unsubstituted methoxyalkyl.

In another embodiment of the compounds of formula IH, M⁴ is —N(R⁵)—, L⁵ is —C(R⁴)₂—, and Z is substituted or unsubstituted methoxymethyl.

In another embodiment of the compounds of formula IH, M⁴ is —N(R⁵)—, L⁵ is —CH₂—, and Z is substituted or unsubstituted methoxymethyl.

In another embodiment of the compounds of formula IH, M⁴ is —N(R⁵)—, L⁵ is —CH(CH₃)—, and Z is substituted or unsubstituted methoxymethyl.

In another embodiment of the compounds of formula IH, M⁴ is —N(R⁵)—, L⁵ is —C(O)—, and Z is substituted or unsubstituted methoxymethyl.

In another embodiment of the compounds of formula IH, M⁴ is —N(R⁵)—, L⁵ is —S(O)₂—, and Z is substituted or unsubstituted methoxymethyl.

In another embodiment of the compounds of formula IH, M⁴ is —N(R⁵)—, L⁵ is —C(R⁴)₂—, and Z is substituted or unsubstituted methoxyethyl.

In another embodiment of the compounds of formula IH, M⁴ is —N(R⁵)—, L⁵ is —CH₂—, and Z is substituted or unsubstituted methoxyethyl.

In another embodiment of the compounds of formula IH, M⁴ is —N(R⁵)—, L⁵ is —CH(CH₃)—, and Z is substituted or unsubstituted methoxyethyl.

In another embodiment of the compounds of formula IH, M⁴ is —N(R⁵)—, L⁵ is —C(O)—, and Z is substituted or unsubstituted methoxyethyl.

In another embodiment of the compounds of formula IH, M⁴ is —N(R⁵)—, L⁵ is —S(O)₂—, and Z is substituted or unsubstituted methoxyethyl.

In another embodiment of the compounds of formula IH, M⁴ is —C(R⁴)₂—.

In another embodiment of the compounds of formula IH, M⁴ is —CH₂—.

In another embodiment of the compounds of formula IH, M⁴ is —CH(CH₃)—.

In another embodiment of the compounds of formula IH, M⁴ is —C(R⁴)₂—, and R¹ is H.

In another embodiment of the compounds of formula IH, M⁴ is —CH₂—, and R¹ is H.

In another embodiment of the compounds of formula IH, M⁴ is —CH(CH₃)—, and R¹ is H.

In another embodiment of the compounds of formula IH, M⁴ is —C(R⁴)₂—, and R¹ is alkyl.

In another embodiment of the compounds of formula IH, M⁴ is —CH₂—, and R¹ is alkyl.

In another embodiment of the compounds of formula IH, M⁴ is —CH(CH₃)—, and R¹ is alkyl.

In another embodiment of the compounds of formula IH, M⁴ is —C(R⁴)₂—, and R¹ is substituted or unsubstituted arylalkyl.

In another embodiment of the compounds of formula IH, M⁴ is —CH₂—, and R¹ is substituted or unsubstituted arylalkyl.

In another embodiment of the compounds of formula IH, M⁴ is —CH(CH₃)—, and R¹ is substituted or unsubstituted arylalkyl.

In another embodiment of the compounds of formula IH, M⁴ is —C(R⁴)₂—, R¹ is H, and L² is a covalent bond.

In another embodiment of the compounds of formula IH, M⁴ is —CH₂—, R¹ is H, and L² is a covalent bond.

In another embodiment of the compounds of formula IH, M⁴ is —CH(CH₃)—, R¹ is H, and L² is a covalent bond.

In another embodiment of the compounds of formula IH, M⁴ is —C(R⁴)₂—, R¹ is alkyl, and L² is a covalent bond.

In another embodiment of the compounds of formula IH, M⁴ is —CH₂—, R¹ is alkyl, and L² is a covalent bond.

In another embodiment of the compounds of formula IH, M⁴ is —CH(CH₃)—, R¹ is alkyl, and L² is a covalent bond.

In another embodiment of the compounds of formula IH, M⁴ is —C(R⁴)₂—, R¹ is substituted or unsubstituted arylalkyl, and L² is a covalent bond.

In another embodiment of the compounds of formula IH, M⁴ is —CH₂—, R¹ is substituted or unsubstituted arylalkyl, and L² is a covalent bond.

In another embodiment of the compounds of formula IH, M⁴ is —CH(CH₃)—, R¹ is substituted or unsubstituted arylalkyl, and L² is a covalent bond.

In another embodiment of the compounds of formula IH, M⁴ is —C(R⁴)₂—, R¹ is H, and L² is —C(R⁴)₂—.

In another embodiment of the compounds of formula IH, M⁴ is —CH₂—, R¹ is H, and L² is —C(R⁴)₂—.

In another embodiment of the compounds of formula IH, M⁴ is —CH(CH₃)—, R¹ is H, and L² is —C(R⁴)₂—

In another embodiment of the compounds of formula IH, M⁴ is —C(R⁴)₂—, R¹ is alkyl, and L² is —C(R⁴)₂—.

In another embodiment of the compounds of formula IH, M⁴ is —CH₂—, R¹ is alkyl, and L² is —C(R⁴)₂—.

In another embodiment of the compounds of formula IH, M⁴ is —CH(CH₃)—, R¹ is alkyl, and L² is —C(R⁴)₂—.

In another embodiment of the compounds of formula IH, M⁴ is —C(R⁴)₂—, R¹ is substituted or unsubstituted arylalkyl, and L² is —C(R⁴)₂—.

In another embodiment of the compounds of formula IH, M⁴ is —CH₂—, R¹ is substituted or unsubstituted arylalkyl, and L² is —C(R⁴)₂—.

In another embodiment of the compounds of formula IH, M⁴ is —CH(CH₃)—, R¹ is substituted or unsubstituted arylalkyl, and L² is —C(R⁴)₂—.

In another embodiment of the compounds of formula IH, M⁴ is —C(R⁴)₂—, R¹ is H, and L² is —C(O)—.

In another embodiment of the compounds of formula IH, M⁴ is —CH₂—, R¹ is H, and L² is —C(O)—.

In another embodiment of the compounds of formula IH, M⁴ is —CH(CH₃)—, R¹ is H, and L² is —C(O)—.

In another embodiment of the compounds of formula IH, M⁴ is —C(R⁴)₂—, R¹ is alkyl, and L² is —C(O)—.

In another embodiment of the compounds of formula IH, M⁴ is —CH₂—, R¹ is alkyl, and L² is —C(O)—.

In another embodiment of the compounds of formula IH, M⁴ is —CH(CH₃)—, R¹ is alkyl, and L² is —C(O)—.

In another embodiment of the compounds of formula IH, M⁴ is —C(R⁴)₂—, R¹ is substituted or unsubstituted arylalkyl, and L² is —C(O)—.

In another embodiment of the compounds of formula IH, M⁴ is —CH₂—, R¹ is substituted or unsubstituted arylalkyl, and L² is —C(O)—.

In another embodiment of the compounds of formula IH, M⁴ is —CH(CH₃)—, R¹ is substituted or unsubstituted arylalkyl, and L² is —C(O)—.

In another embodiment of the compounds of formula IH, M⁴ is —C(R⁴)₂—, R¹ is H, and L² is —S(O)₂—.

In another embodiment of the compounds of formula IH, M⁴ is —CH₂—, R¹ is H, and L² is —S(O)₂—.

In another embodiment of the compounds of formula IH, M⁴ is —CH(CH₃)—, R¹ is H, and L² is —S(O)₂—.

In another embodiment of the compounds of formula IH, M⁴ is —C(R⁴)₂—, R¹ is alkyl, and L² is —S(O)₂—.

In another embodiment of the compounds of formula IH, M⁴ is —CH₂—, R¹ is alkyl, and L² is —S(O)₂—.

In another embodiment of the compounds of formula IH, M⁴ is —CH(CH₃)—, R¹ is alkyl, and L² is —S(O)₂—.

In another embodiment of the compounds of formula IH, M⁴ is —C(R⁴)₂—, R¹ is substituted or unsubstituted arylalkyl, and L² is —S(O)₂—.

In another embodiment of the compounds of formula IH, M⁴ is —CH₂—, R¹ is substituted or unsubstituted arylalkyl, and L² is —S(O)₂—.

In another embodiment of the compounds of formula IH, M⁴ is —CH(CH₃)—, R¹ is substituted or unsubstituted arylalkyl, and L² is —S(O)₂—.

In another embodiment of the compounds of formula IH, M⁴ is —C(R⁴)₂—, R¹ is H, L² is a covalent bond, and X is substituted or unsubstituted aryl.

In another embodiment of the compounds of formula IH, M⁴ is —CH₂—, R¹ is H, L² is a covalent bond, and X is substituted or unsubstituted aryl.

In another embodiment of the compounds of formula IH, M⁴ is —CH(CH₃)—, R¹ is H, L² is a covalent bond, and X is substituted or unsubstituted aryl.

In another embodiment of the compounds of formula IH, M⁴ is —C(R⁴)₂—, R¹ is alkyl, L² is a covalent bond, and X is substituted or unsubstituted aryl.

In another embodiment of the compounds of formula IH, M⁴ is —CH₂—, R¹ is alkyl, L² is a covalent bond, and X is substituted or unsubstituted aryl.

In another embodiment of the compounds of formula IH, M⁴ is —CH(CH₃)—, R¹ is alkyl, L² is a covalent bond, and X is substituted or unsubstituted aryl.

In another embodiment of the compounds of formula IH, M⁴ is —C(R⁴)₂—, R¹ is substituted or unsubstituted arylalkyl, L² is a covalent bond, and X is substituted or unsubstituted aryl.

In another embodiment of the compounds of formula IH, M⁴ is —CH₂—, R¹ is substituted or unsubstituted arylalkyl, L² is a covalent bond, and X is substituted or unsubstituted aryl.

In another embodiment of the compounds of formula IH, M⁴ is —CH(CH₃)—, R¹ is substituted or unsubstituted arylalkyl, L² is a covalent bond, and X is substituted or unsubstituted aryl.

In another embodiment of the compounds of formula IH, M⁴ is —C(R⁴)₂—, R¹ is H, L² is —C(R⁴)₂—, and X is substituted or unsubstituted aryl.

In another embodiment of the compounds of formula IH, M⁴ is —CH₂—, R¹ is H, L² is —C(R⁴)₂—, and X is substituted or unsubstituted aryl.

In another embodiment of the compounds of formula IH, M⁴ is —CH(CH₃)—, R¹ is H, L² is —C(R⁴)₂—, and X is substituted or unsubstituted aryl.

In another embodiment of the compounds of formula IH, M⁴ is —C(R⁴)₂—, R¹ is alkyl, L² is —C(R⁴)₂—, and X is substituted or unsubstituted aryl.

In another embodiment of the compounds of formula IH, M⁴ is —CH₂—, R¹ is alkyl, L² is —C(R⁴)₂—, and X is substituted or unsubstituted aryl.

In another embodiment of the compounds of formula IH, M⁴ is —CH(CH₃)—, R¹ is alkyl, L² is —C(R⁴)₂—, and X is substituted or unsubstituted aryl.

In another embodiment of the compounds of formula IH, M⁴ is —C(R⁴)₂—, R¹ is substituted or unsubstituted arylalkyl, L² is —C(R⁴)₂—, and X is substituted or unsubstituted aryl.

In another embodiment of the compounds of formula IH, M⁴ is —CH₂—, R¹ is substituted or unsubstituted arylalkyl, L² is —C(R⁴)₂—, and X is substituted or unsubstituted aryl.

In another embodiment of the compounds of formula IH, M⁴ is —CH(CH₃)—, R¹ is substituted or unsubstituted arylalkyl, L² is —C(R⁴)₂—, and X is substituted or unsubstituted aryl.

In another embodiment of the compounds of formula IH, M⁴ is —C(R⁴)₂—, R¹ is H, L² is —C(O)—, and X is substituted or unsubstituted aryl.

In another embodiment of the compounds of formula IH, M⁴ is —CH₂—, R¹ is H, L² is —C(O)—, and X is substituted or unsubstituted aryl.

In another embodiment of the compounds of formula IH, M⁴ is —CH(CH₃)—, R¹ is H, L² is —C(O)—, and X is substituted or unsubstituted aryl.

In another embodiment of the compounds of formula IH, M⁴ is —C(R⁴)₂—, R¹ is alkyl, L² is —C(O)—, and X is substituted or unsubstituted aryl.

In another embodiment of the compounds of formula IH, M⁴ is —CH₂—, R¹ is alkyl, L² is —C(O)—, and X is substituted or unsubstituted aryl.

In another embodiment of the compounds of formula IH, M⁴ is —CH(CH₃)—, R¹ is alkyl, L² is —C(O)—, and X is substituted or unsubstituted aryl.

In another embodiment of the compounds of formula IH, M⁴ is —C(R⁴)₂—, R¹ is substituted or unsubstituted arylalkyl, L² is —C(O)—, and X is substituted or unsubstituted aryl.

In another embodiment of the compounds of formula IH, M⁴ is —CH₂—, R¹ is substituted or unsubstituted arylalkyl, L² is —C(O)—, and X is substituted or unsubstituted aryl.

In another embodiment of the compounds of formula IH, M⁴ is —CH(CH₃)—, R¹ is substituted or unsubstituted arylalkyl, L² is —C(O)—, and X is substituted or unsubstituted aryl.

In another embodiment of the compounds of formula IH, M⁴ is —C(R⁴)₂—, R¹ is H, L² is —S(O)₂—, and X is substituted or unsubstituted aryl.

In another embodiment of the compounds of formula IH, M⁴ is —CH₂—, R¹ is H, L² is —S(O)₂—, and X is substituted or unsubstituted aryl.

In another embodiment of the compounds of formula IH, M⁴ is —CH(CH₃)—, R¹ is H, L² is —S(O)₂—, and X is substituted or unsubstituted aryl.

In another embodiment of the compounds of formula IH, M⁴ is —C(R⁴)₂—, R¹ is alkyl, L² is —S(O)₂—, and X is substituted or unsubstituted aryl.

In another embodiment of the compounds of formula IH, M⁴ is —CH₂—, R¹ is alkyl, L² is —S(O)₂—, and X is substituted or unsubstituted aryl.

In another embodiment of the compounds of formula IH, M⁴ is —CH(CH₃)—, R¹ is alkyl, L² is —S(O)₂—, and X is substituted or unsubstituted aryl.

In another embodiment of the compounds of formula IH, M⁴ is —C(R⁴)₂—, R¹ is substituted or unsubstituted arylalkyl, L² is —S(O)₂—, and X is substituted or unsubstituted aryl.

In another embodiment of the compounds of formula IH, M⁴ is —CH₂—, R¹ is substituted or unsubstituted arylalkyl, L² is —S(O)₂—, and X is substituted or unsubstituted aryl.

In another embodiment of the compounds of formula IH, M⁴ is —CH(CH₃)—, R¹ is substituted or unsubstituted arylalkyl, L² is —S(O)₂—, and X is substituted or unsubstituted aryl.

In another embodiment of the compounds of formula IH, M⁴ is —C(R⁴)₂—, R¹ is H, L² is a covalent bond, and X is substituted or unsubstituted phenyl.

In another embodiment of the compounds of formula IH, M⁴ is —CH₂—, R¹ is H, L² is a covalent bond, and X is substituted or unsubstituted phenyl.

In another embodiment of the compounds of formula IH, M⁴ is —CH(CH₃)—, R¹ is H, L² is a covalent bond, and X is substituted or unsubstituted phenyl.

In another embodiment of the compounds of formula IH, M⁴ is —C(R⁴)₂—, R¹ is alkyl, L² is a covalent bond, and X is substituted or unsubstituted phenyl.

In another embodiment of the compounds of formula IH, M⁴ is —CH₂—, R¹ is alkyl, L² is a covalent bond, and X is substituted or unsubstituted phenyl.

In another embodiment of the compounds of formula IH, M⁴ is —CH(CH₃)—, R¹ is alkyl, L² is a covalent bond, and X is substituted or unsubstituted phenyl.

In another embodiment of the compounds of formula IH, M⁴ is —C(R⁴)₂—, R¹ is substituted or unsubstituted arylalkyl, L² is a covalent bond, and X is substituted or unsubstituted phenyl.

In another embodiment of the compounds of formula IH, M⁴ is —CH₂—, R¹ is substituted or unsubstituted arylalkyl, L² is a covalent bond, and X is substituted or unsubstituted phenyl.

In another embodiment of the compounds of formula IH, M⁴ is —CH(CH₃)—, R¹ is substituted or unsubstituted arylalkyl, L² is a covalent bond, and X is substituted or unsubstituted phenyl.

In another embodiment of the compounds of formula IH, M⁴ is —C(R⁴)₂—, R¹ is H, L² is —C(R⁴)₂—, and X is substituted or unsubstituted phenyl.

In another embodiment of the compounds of formula IH, M⁴ is —CH₂—, R¹ is H, L² is —C(R⁴)₂—, and X is substituted or unsubstituted phenyl.

In another embodiment of the compounds of formula IH, M⁴ is —CH(CH₃)—, R¹ is H, L² is —C(R⁴)₂—, and X is substituted or unsubstituted phenyl.

In another embodiment of the compounds of formula IH, M⁴ is —C(R⁴)₂—, R¹ is alkyl, L² is —C(R⁴)₂—, and X is substituted or unsubstituted phenyl.

In another embodiment of the compounds of formula IH, M⁴ is —CH₂—, R¹ is alkyl, L² is —C(R⁴)₂—, and X is substituted or unsubstituted phenyl.

In another embodiment of the compounds of formula IH, M⁴ is —CH(CH₃)—, R¹ is alkyl, L² is —C(R⁴)₂—, and X is substituted or unsubstituted phenyl.

In another embodiment of the compounds of formula IH, M⁴ is —C(R⁴)₂—, R¹ is substituted or unsubstituted arylalkyl, L² is —C(R⁴)₂—, and X is substituted or unsubstituted phenyl.

In another embodiment of the compounds of formula IH, M⁴ is —CH₂—, R¹ is substituted or unsubstituted arylalkyl, L² is —C(R⁴)₂—, and X is substituted or unsubstituted phenyl.

In another embodiment of the compounds of formula IH, M⁴ is —CH(CH₃)—, R¹ is substituted or unsubstituted arylalkyl, L² is —C(R⁴)₂—, and X is substituted or unsubstituted phenyl.

In another embodiment of the compounds of formula IH, M⁴ is —C(R⁴)₂—, R¹ is H, L² is —C(O)—, and X is substituted or unsubstituted phenyl.

In another embodiment of the compounds of formula IH, M⁴ is —CH₂—, R¹ is H, L² is —C(O)—, and X is substituted or unsubstituted phenyl.

In another embodiment of the compounds of formula IH, M⁴ is —CH(CH₃)—, R¹ is H, L² is —C(O)—, and X is substituted or unsubstituted phenyl.

In another embodiment of the compounds of formula IH, M⁴ is —C(R⁴)₂—, R¹ is alkyl, L² is —C(O)—, and X is substituted or unsubstituted phenyl.

In another embodiment of the compounds of formula IH, M⁴ is —CH₂—, R¹ is alkyl, L² is —C(O)—, and X is substituted or unsubstituted phenyl.

In another embodiment of the compounds of formula IH, M⁴ is —CH(CH₃)—, R¹ is alkyl, L² is —C(O)—, and X is substituted or unsubstituted phenyl.

In another embodiment of the compounds of formula IH, M⁴ is —C(R⁴)₂—, R¹ is substituted or unsubstituted arylalkyl, L² is —C(O)—, and X is substituted or unsubstituted phenyl.

In another embodiment of the compounds of formula IH, M⁴ is —CH₂—, R¹ is substituted or unsubstituted arylalkyl, L² is —C(O)—, and X is substituted or unsubstituted phenyl.

In another embodiment of the compounds of formula IH, M⁴ is —CH(CH₃)—, R¹ is substituted or unsubstituted arylalkyl, L² is —C(O)—, and X is substituted or unsubstituted phenyl.

In another embodiment of the compounds of formula IH, M⁴ is —C(R⁴)₂—, R¹ is H, L² is —S(O)₂—, and X is substituted or unsubstituted phenyl.

In another embodiment of the compounds of formula IH, M⁴ is —CH₂—, R¹ is H, L² is —S(O)₂—, and X is substituted or unsubstituted phenyl.

In another embodiment of the compounds of formula IH, M⁴ is —CH(CH₃)—, R¹ is H, L² is —S(O)₂—, and X is substituted or unsubstituted phenyl.

In another embodiment of the compounds of formula IH, M⁴ is —C(R⁴)₂—, R¹ is alkyl, L² is —S(O)₂—, and X is substituted or unsubstituted phenyl.

In another embodiment of the compounds of formula IH, M⁴ is —CH₂—, R¹ is alkyl, L² is —S(O)₂—, and X is substituted or unsubstituted phenyl.

In another embodiment of the compounds of formula IH, M⁴ is —CH(CH₃)—, R¹ is alkyl, L² is —S(O)₂—, and X is substituted or unsubstituted phenyl.

In another embodiment of the compounds of formula IH, M⁴ is —C(R⁴)₂—, R¹ is substituted or unsubstituted arylalkyl, L² is —S(O)₂—, and X is substituted or unsubstituted phenyl.

In another embodiment of the compounds of formula IH, M⁴ is —CH₂—, R¹ is substituted or unsubstituted arylalkyl, L² is —S(O)₂—, and X is substituted or unsubstituted phenyl.

In another embodiment of the compounds of formula IH, M⁴ is —CH(CH₃)—, R¹ is substituted or unsubstituted arylalkyl, L² is —S(O)₂—, and X is substituted or unsubstituted phenyl.

In another embodiment of the compounds of formula IH, M⁴ is —C(R⁴)₂—, R¹ is H, L² is a covalent bond, and X is substituted or unsubstituted heteroaryl.

In another embodiment of the compounds of formula IH, M⁴ is —CH₂—, R¹ is H, L² is a covalent bond, and X is substituted or unsubstituted heteroaryl.

In another embodiment of the compounds of formula IH, M⁴ is —CH(CH₃)—, R¹ is H, L² is a covalent bond, and X is substituted or unsubstituted heteroaryl.

In another embodiment of the compounds of formula IH, M⁴ is —C(R⁴)₂—, R¹ is alkyl, L² is a covalent bond, and X is substituted or unsubstituted heteroaryl.

In another embodiment of the compounds of formula IH, M⁴ is —CH₂—, R¹ is alkyl, L² is a covalent bond, and X is substituted or unsubstituted heteroaryl.

In another embodiment of the compounds of formula IH, M⁴ is —CH(CH₃)—, R¹ is alkyl, L² is a covalent bond, and X is substituted or unsubstituted heteroaryl.

In another embodiment of the compounds of formula IH, M⁴ is —C(R⁴)₂—, R¹ is substituted or unsubstituted arylalkyl, L² is a covalent bond, and X is substituted or unsubstituted heteroaryl.

In another embodiment of the compounds of formula IH, M⁴ is —CH₂—, R¹ is substituted or unsubstituted arylalkyl, L² is a covalent bond, and X is substituted or unsubstituted heteroaryl.

In another embodiment of the compounds of formula IH, M⁴ is —CH(CH₃)—, R¹ is substituted or unsubstituted arylalkyl, L² is a covalent bond, and X is substituted or unsubstituted heteroaryl.

In another embodiment of the compounds of formula IH, M⁴ is —C(R⁴)₂—, R¹ is H, L² is —C(R⁴)₂—, and X is substituted or unsubstituted heteroaryl.

In another embodiment of the compounds of formula IH, M⁴ is —CH₂—, R¹ is H, L² is —C(R⁴)₂—, and X is substituted or unsubstituted heteroaryl.

In another embodiment of the compounds of formula IH, M⁴ is —CH(CH₃)—, R¹ is H, L² is —C(R⁴)₂—, and X is substituted or unsubstituted heteroaryl.

In another embodiment of the compounds of formula IH, M⁴ is —C(R⁴)₂—, R¹ is alkyl, L² is —C(R⁴)₂—, and X is substituted or unsubstituted heteroaryl.

In another embodiment of the compounds of formula IH, M⁴ is —CH₂—, R¹ is alkyl, L² is —C(R⁴)₂—, and X is substituted or unsubstituted heteroaryl.

In another embodiment of the compounds of formula IH, M⁴ is —CH(CH₃)—, R¹ is alkyl, L² is —C(R⁴)₂—, and X is substituted or unsubstituted heteroaryl.

In another embodiment of the compounds of formula IH, M⁴ is —C(R⁴)₂—, R¹ is substituted or unsubstituted arylalkyl, L² is —C(R⁴)₂—, and X is substituted or unsubstituted heteroaryl.

In another embodiment of the compounds of formula IH, M⁴ is —CH₂—, R¹ is substituted or unsubstituted arylalkyl, L² is —C(R⁴)₂—, and X is substituted or unsubstituted heteroaryl.

In another embodiment of the compounds of formula IH, M⁴ is —CH(CH₃)—, R¹ is substituted or unsubstituted arylalkyl, L² is —C(R⁴)₂—, and X is substituted or unsubstituted heteroaryl.

In another embodiment of the compounds of formula IH, M⁴ is —C(R⁴)₂—, R¹ is H, L² is —C(O)—, and X is substituted or unsubstituted heteroaryl.

In another embodiment of the compounds of formula IH, M⁴ is —CH₂—, R¹ is H, L² is —C(O)—, and X is substituted or unsubstituted heteroaryl.

In another embodiment of the compounds of formula IH, M⁴ is —CH(CH₃)—, R¹ is H, L² is —C(O)—, and X is substituted or unsubstituted heteroaryl.

In another embodiment of the compounds of formula IH, M⁴ is —C(R⁴)₂—, R¹ is alkyl, L² is —C(O)—, and X is substituted or unsubstituted heteroaryl.

In another embodiment of the compounds of formula IH, M⁴ is —CH₂—, R¹ is alkyl, L² is —C(O)—, and X is substituted or unsubstituted heteroaryl.

In another embodiment of the compounds of formula IH, M⁴ is —CH(CH₃)—, R¹ is alkyl, L² is —C(O)—, and X is substituted or unsubstituted heteroaryl.

In another embodiment of the compounds of formula IH, M⁴ is —C(R⁴)₂—, R¹ is substituted or unsubstituted arylalkyl, L² is —C(O)—, and X is substituted or unsubstituted heteroaryl.

In another embodiment of the compounds of formula IH, M⁴ is —CH₂—, R¹ is substituted or unsubstituted arylalkyl, L² is —C(O)—, and X is substituted or unsubstituted heteroaryl.

In another embodiment of the compounds of formula IH, M⁴ is —CH(CH₃)—, R¹ is substituted or unsubstituted arylalkyl, L² is —C(O)—, and X is substituted or unsubstituted heteroaryl.

In another embodiment of the compounds of formula IH, M⁴ is —C(R⁴)₂—, R¹ is H, L² is —S(O)₂—, and X is substituted or unsubstituted heteroaryl.

In another embodiment of the compounds of formula IH, M⁴ is —CH₂—, R¹ is H, L² is —S(O)₂—, and X is substituted or unsubstituted heteroaryl.

In another embodiment of the compounds of formula IH, M⁴ is —CH(CH₃)—, R¹ is H, L² is —S(O)₂—, and X is substituted or unsubstituted heteroaryl.

In another embodiment of the compounds of formula IH, M⁴ is —C(R⁴)₂—, R¹ is alkyl, L² is —S(O)₂—, and X is substituted or unsubstituted heteroaryl.

In another embodiment of the compounds of formula IH, M⁴ is —CH₂—, R¹ is alkyl, L² is —S(O)₂—, and X is substituted or unsubstituted heteroaryl.

In another embodiment of the compounds of formula IH, M⁴ is —CH(CH₃)—, R¹ is alkyl, L² is —S(O)₂—, and X is substituted or unsubstituted heteroaryl.

In another embodiment of the compounds of formula IH, M⁴ is —C(R⁴)₂—, R¹ is substituted or unsubstituted arylalkyl, L² is —S(O)₂—, and X is substituted or unsubstituted heteroaryl.

In another embodiment of the compounds of formula IH, M⁴ is —CH₂—, R¹ is substituted or unsubstituted arylalkyl, L² is —S(O)₂—, and X is substituted or unsubstituted heteroaryl.

In another embodiment of the compounds of formula IH, M⁴ is —CH(CH₃)—, R¹ is substituted or unsubstituted arylalkyl, L² is —S(O)₂—, and X is substituted or unsubstituted heteroaryl.

In another embodiment of the compounds of formula IH, M⁴ is —C(R⁴)₂—, R¹ is H, L² is a covalent bond, and X is substituted or unsubstituted pyridyl.

In another embodiment of the compounds of formula IH, M⁴ is —CH₂—, R¹ is H, L² is a covalent bond, and X is substituted or unsubstituted pyridyl.

In another embodiment of the compounds of formula IH, M⁴ is —CH(CH₃)—, R¹ is H, L² is a covalent bond, and X is substituted or unsubstituted pyridyl.

In another embodiment of the compounds of formula IH, M⁴ is —C(R⁴)₂—, R¹ is alkyl, L² is a covalent bond, and X is substituted or unsubstituted pyridyl.

In another embodiment of the compounds of formula IH, M⁴ is —CH₂—, R¹ is alkyl, L² is a covalent bond, and X is substituted or unsubstituted pyridyl.

In another embodiment of the compounds of formula IH, M⁴ is —CH(CH₃)—, R¹ is alkyl, L² is a covalent bond, and X is substituted or unsubstituted pyridyl.

In another embodiment of the compounds of formula IH, M⁴ is —C(R⁴)₂—, R¹ is substituted or unsubstituted arylalkyl, L² is a covalent bond, and X is substituted or unsubstituted pyridyl.

In another embodiment of the compounds of formula IH, M⁴ is —CH₂—, R¹ is substituted or unsubstituted arylalkyl, L² is a covalent bond, and X is substituted or unsubstituted pyridyl.

In another embodiment of the compounds of formula IH, M⁴ is —CH(CH₃)—, R¹ is substituted or unsubstituted arylalkyl, L² is a covalent bond, and X is substituted or unsubstituted pyridyl.

In another embodiment of the compounds of formula IH, M⁴ is —C(R⁴)₂—, R¹ is H, L² is —C(R⁴)₂—, and X is substituted or unsubstituted pyridyl.

In another embodiment of the compounds of formula IH, M⁴ is —CH₂—, R¹ is H, L² is —C(R⁴)₂—, and X is substituted or unsubstituted pyridyl.

In another embodiment of the compounds of formula IH, M⁴ is —CH(CH₃)—, R¹ is H, L² is —C(R⁴)₂—, and X is substituted or unsubstituted pyridyl.

In another embodiment of the compounds of formula IH, M⁴ is —C(R⁴)₂—, R¹ is alkyl, L² is —C(R⁴)₂—, and X is substituted or unsubstituted pyridyl.

In another embodiment of the compounds of formula IH, M⁴ is —CH₂—, R¹ is alkyl, L² is —C(R⁴)₂—, and X is substituted or unsubstituted pyridyl.

In another embodiment of the compounds of formula IH, M⁴ is —CH(CH₃)—, R¹ is alkyl, L² is —C(R⁴)₂—, and X is substituted or unsubstituted pyridyl.

In another embodiment of the compounds of formula IH, M⁴ is —C(R⁴)₂—, R¹ is substituted or unsubstituted arylalkyl, L² is —C(R⁴)₂—, and X is substituted or unsubstituted pyridyl.

In another embodiment of the compounds of formula IH, M⁴ is —CH₂—, R¹ is substituted or unsubstituted arylalkyl, L² is —C(R⁴)₂—, and X is substituted or unsubstituted pyridyl.

In another embodiment of the compounds of formula IH, M⁴ is —CH(CH₃)—, R¹ is substituted or unsubstituted arylalkyl, L² is —C(R⁴)₂—, and X is substituted or unsubstituted pyridyl.

In another embodiment of the compounds of formula IH, M⁴ is —C(R⁴)₂—, R¹ is H, L² is —C(O)—, and X is substituted or unsubstituted pyridyl.

In another embodiment of the compounds of formula IH, M⁴ is —CH₂—, R¹ is H, L² is —C(O)—, and X is substituted or unsubstituted pyridyl.

In another embodiment of the compounds of formula IH, M⁴ is —CH(CH₃)—, R¹ is H, L² is —C(O)—, and X is substituted or unsubstituted pyridyl.

In another embodiment of the compounds of formula IH, M⁴ is —C(R⁴)₂—, R¹ is alkyl, L² is —C(O)—, and X is substituted or unsubstituted pyridyl.

In another embodiment of the compounds of formula IH, M⁴ is —CH₂—, R¹ is alkyl, L² is —C(O)—, and X is substituted or unsubstituted pyridyl.

In another embodiment of the compounds of formula IH, M⁴ is —CH(CH₃)—, R¹ is alkyl, L² is —C(O)—, and X is substituted or unsubstituted pyridyl.

In another embodiment of the compounds of formula IH, M⁴ is —C(R⁴)₂—, R¹ is substituted or unsubstituted arylalkyl, L² is —C(O)—, and X is substituted or unsubstituted pyridyl.

In another embodiment of the compounds of formula IH, M⁴ is —CH₂—, R¹ is substituted or unsubstituted arylalkyl, L² is —C(O)—, and X is substituted or unsubstituted pyridyl.

In another embodiment of the compounds of formula IH, M⁴ is —CH(CH₃)—, R¹ is substituted or unsubstituted arylalkyl, L² is —C(O)—, and X is substituted or unsubstituted pyridyl.

In another embodiment of the compounds of formula IH, M⁴ is —C(R⁴)₂—, R¹ is H, L² is —S(O)₂—, and X is substituted or unsubstituted pyridyl.

In another embodiment of the compounds of formula IH, M⁴ is —CH₂—, R¹ is H, L² is —S(O)₂—, and X is substituted or unsubstituted pyridyl.

In another embodiment of the compounds of formula IH, M⁴ is —CH(CH₃)—, R¹ is H, L² is —S(O)₂—, and X is substituted or unsubstituted pyridyl.

In another embodiment of the compounds of formula IH, M⁴ is —C(R⁴)₂—, R¹ is alkyl, L² is —S(O)₂—, and X is substituted or unsubstituted pyridyl.

In another embodiment of the compounds of formula IH, M⁴ is —CH₂—, R¹ is alkyl, L² is —S(O)₂—, and X is substituted or unsubstituted pyridyl.

In another embodiment of the compounds of formula IH, M⁴ is —CH(CH₃)—, R¹ is alkyl, L² is —S(O)₂—, and X is substituted or unsubstituted pyridyl.

In another embodiment of the compounds of formula IH, M⁴ is —C(R⁴)₂—, R¹ is substituted or unsubstituted arylalkyl, L² is —S(O)₂—, and X is substituted or unsubstituted pyridyl.

In another embodiment of the compounds of formula IH, M⁴ is —CH₂—, R¹ is substituted or unsubstituted arylalkyl, L² is —S(O)₂—, and X is substituted or unsubstituted pyridyl.

In another embodiment of the compounds of formula IH, M⁴ is —CH(CH₃)—, R¹ is substituted or unsubstituted arylalkyl, L² is —S(O)₂—, and X is substituted or unsubstituted pyridyl.

In another embodiment of the compounds of formula IH, M⁴ is —C(R⁴)₂—, R¹ is H, L² is a covalent bond, and X is substituted or unsubstituted pyrimidyl.

In another embodiment of the compounds of formula IH, M⁴ is —CH₂—, R¹ is H, L² is a covalent bond, and X is substituted or unsubstituted pyrimidyl.

In another embodiment of the compounds of formula IH, M⁴ is —CH(CH₃)—, R¹ is H, L² is a covalent bond, and X is substituted or unsubstituted pyrimidyl.

In another embodiment of the compounds of formula IH, M⁴ is —C(R⁴)₂—, R¹ is alkyl, L² is a covalent bond, and X is substituted or unsubstituted pyrimidyl.

In another embodiment of the compounds of formula IH, M⁴ is —CH₂—, R¹ is alkyl, L² is a covalent bond, and X is substituted or unsubstituted pyrimidyl.

In another embodiment of the compounds of formula IH, M⁴ is —CH(CH₃)—, R¹ is alkyl, L² is a covalent bond, and X is substituted or unsubstituted pyrimidyl.

In another embodiment of the compounds of formula IH, M⁴ is —C(R⁴)₂—, R¹ is substituted or unsubstituted arylalkyl, L² is a covalent bond, and X is substituted or unsubstituted pyrimidyl.

In another embodiment of the compounds of formula IH, M⁴ is —CH₂—, R¹ is substituted or unsubstituted arylalkyl, L² is a covalent bond, and X is substituted or unsubstituted pyrimidyl.

In another embodiment of the compounds of formula IH, M⁴ is —CH(CH₃)—, R¹ is substituted or unsubstituted arylalkyl, L² is a covalent bond, and X is substituted or unsubstituted pyrimidyl.

In another embodiment of the compounds of formula IH, M⁴ is —C(R⁴)₂—, R¹ is H, L² is —C(R⁴)₂—, and X is substituted or unsubstituted pyrimidyl.

In another embodiment of the compounds of formula IH, M⁴ is —CH₂—, R¹ is H, L² is —C(R⁴)₂—, and X is substituted or unsubstituted pyrimidyl.

In another embodiment of the compounds of formula IH, M⁴ is —CH(CH₃)—, R¹ is H, L² is —C(R⁴)₂—, and X is substituted or unsubstituted pyrimidyl.

In another embodiment of the compounds of formula IH, M⁴ is —C(R⁴)₂—, R¹ is alkyl, L² is —C(R⁴)₂—, and X is substituted or unsubstituted pyrimidyl.

In another embodiment of the compounds of formula IH, M⁴ is —CH₂—, R¹ is alkyl, L² is —C(R⁴)₂—, and X is substituted or unsubstituted pyrimidyl.

In another embodiment of the compounds of formula IH, M⁴ is —CH(CH₃)—, R¹ is alkyl, L² is —C(R⁴)₂—, and X is substituted or unsubstituted pyrimidyl.

In another embodiment of the compounds of formula IH, M⁴ is —C(R⁴)₂—, R¹ is substituted or unsubstituted arylalkyl, L² is —C(R⁴)₂—, and X is substituted or unsubstituted pyrimidyl.

In another embodiment of the compounds of formula IH, M⁴ is —CH₂—, R¹ is substituted or unsubstituted arylalkyl, L² is —C(R⁴)₂—, and X is substituted or unsubstituted pyrimidyl.

In another embodiment of the compounds of formula IH, M⁴ is —CH(CH₃)—, R¹ is substituted or unsubstituted arylalkyl, L² is —C(R⁴)₂—, and X is substituted or unsubstituted pyrimidyl.

In another embodiment of the compounds of formula IH, M⁴ is —C(R⁴)₂—, R¹ is H, L² is —C(O)—, and X is substituted or unsubstituted pyrimidyl.

In another embodiment of the compounds of formula IH, M⁴ is —CH₂—, R¹ is H, L² is —C(O)—, and X is substituted or unsubstituted pyrimidyl.

In another embodiment of the compounds of formula IH, M⁴ is —CH(CH₃)—, R¹ is H, L² is —C(O)—, and X is substituted or unsubstituted pyrimidyl.

In another embodiment of the compounds of formula IH, M⁴ is —C(R⁴)₂—, R¹ is alkyl, L² is —C(O)—, and X is substituted or unsubstituted pyrimidyl.

In another embodiment of the compounds of formula IH, M⁴ is —CH₂—, R¹ is alkyl, L² is —C(O)—, and X is substituted or unsubstituted pyrimidyl.

In another embodiment of the compounds of formula IH, M⁴ is —CH(CH₃)—, R¹ is alkyl, L² is —C(O)—, and X is substituted or unsubstituted pyrimidyl.

In another embodiment of the compounds of formula IH, M⁴ is —C(R⁴)₂—, R¹ is substituted or unsubstituted arylalkyl, L² is —C(O)—, and X is substituted or unsubstituted pyrimidyl.

In another embodiment of the compounds of formula IH, M⁴ is —CH₂—, R¹ is substituted or unsubstituted arylalkyl, L² is —C(O)—, and X is substituted or unsubstituted pyrimidyl.

In another embodiment of the compounds of formula IH, M⁴ is —CH(CH₃)—, R¹ is substituted or unsubstituted arylalkyl, L² is —C(O)—, and X is substituted or unsubstituted pyrimidyl.

In another embodiment of the compounds of formula IH, M⁴ is —C(R⁴)₂—, R¹ is H, L² is —S(O)₂—, and X is substituted or unsubstituted pyrimidyl.

In another embodiment of the compounds of formula IH, M⁴ is —CH₂—, R¹ is H, L² is —S(O)₂—, and X is substituted or unsubstituted pyrimidyl.

In another embodiment of the compounds of formula IH, M⁴ is —CH(CH₃)—, R¹ is H, L² is —S(O)₂—, and X is substituted or unsubstituted pyrimidyl.

In another embodiment of the compounds of formula IH, M⁴ is —C(R⁴)₂—, R¹ is alkyl, L² is —S(O)₂—, and X is substituted or unsubstituted pyrimidyl.

In another embodiment of the compounds of formula IH, M⁴ is —CH₂—, R¹ is alkyl, L² is —S(O)₂—, and X is substituted or unsubstituted pyrimidyl.

In another embodiment of the compounds of formula IH, M⁴ is —CH(CH₃)—, R¹ is alkyl, L² is —S(O)₂—, and X is substituted or unsubstituted pyrimidyl.

In another embodiment of the compounds of formula IH, M⁴ is —C(R⁴)₂—, R¹ is substituted or unsubstituted arylalkyl, L² is —S(O)₂—, and X is substituted or unsubstituted pyrimidyl.

In another embodiment of the compounds of formula IH, M⁴ is —CH₂—, R¹ is substituted or unsubstituted arylalkyl, L² is —S(O)₂—, and X is substituted or unsubstituted pyrimidyl.

In another embodiment of the compounds of formula IH, M⁴ is —CH(CH₃)—, R¹ is substituted or unsubstituted arylalkyl, L² is —S(O)₂—, and X is substituted or unsubstituted pyrimidyl.

In another embodiment of the compounds of formula IH, M⁴ is —C(R⁴)₂—, R¹ is H, L² is a covalent bond, and X is substituted or unsubstituted indolyl.

In another embodiment of the compounds of formula IH, M⁴ is —CH₂—, R¹ is H, L² is a covalent bond, and X is substituted or unsubstituted indolyl.

In another embodiment of the compounds of formula IH, M⁴ is —CH(CH₃)—, R¹ is H, L² is a covalent bond, and X is substituted or unsubstituted indolyl.

In another embodiment of the compounds of formula IH, M⁴ is —C(R⁴)₂—, R¹ is alkyl, L² is a covalent bond, and X is substituted or unsubstituted indolyl.

In another embodiment of the compounds of formula IH, M⁴ is —CH₂—, R¹ is alkyl, L² is a covalent bond, and X is substituted or unsubstituted indolyl.

In another embodiment of the compounds of formula IH, M⁴ is —CH(CH₃)—, R¹ is alkyl, L² is a covalent bond, and X is substituted or unsubstituted indolyl.

In another embodiment of the compounds of formula IH, M⁴ is —C(R⁴)₂—, R¹ is substituted or unsubstituted arylalkyl, L² is a covalent bond, and X is substituted or unsubstituted indolyl.

In another embodiment of the compounds of formula IH, M⁴ is —CH₂—, R¹ is substituted or unsubstituted arylalkyl, L² is a covalent bond, and X is substituted or unsubstituted indolyl.

In another embodiment of the compounds of formula IH, M⁴ is —CH(CH₃)—, R¹ is substituted or unsubstituted arylalkyl, L² is a covalent bond, and X is substituted or unsubstituted indolyl.

In another embodiment of the compounds of formula IH, M⁴ is —C(R⁴)₂—, R¹ is H, L² is —C(R⁴)₂—, and X is substituted or unsubstituted indolyl.

In another embodiment of the compounds of formula IH, M⁴ is —CH₂—, R¹ is H, L² is —C(R⁴)₂—, and X is substituted or unsubstituted indolyl.

In another embodiment of the compounds of formula IH, M⁴ is —CH(CH₃)—, R¹ is H, L² is —C(R⁴)₂—, and X is substituted or unsubstituted indolyl.

In another embodiment of the compounds of formula IH, M⁴ is —C(R⁴)₂—, R¹ is alkyl, L² is —C(R⁴)₂—, and X is substituted or unsubstituted indolyl.

In another embodiment of the compounds of formula IH, M⁴ is —CH₂—, R¹ is alkyl, L² is —C(R⁴)₂—, and X is substituted or unsubstituted indolyl.

In another embodiment of the compounds of formula IH, M⁴ is —CH(CH₃)—, R¹ is alkyl, L² is —C(R⁴)₂—, and X is substituted or unsubstituted indolyl.

In another embodiment of the compounds of formula IH, M⁴ is —C(R⁴)₂—, R¹ is substituted or unsubstituted arylalkyl, L² is —C(R⁴)₂—, and X is substituted or unsubstituted indolyl.

In another embodiment of the compounds of formula IH, M⁴ is —CH₂—, R¹ is substituted or unsubstituted arylalkyl, L² is —C(R⁴)₂—, and X is substituted or unsubstituted indolyl.

In another embodiment of the compounds of formula IH, M⁴ is —CH(CH₃)—, R¹ is substituted or unsubstituted arylalkyl, L² is —C(R⁴)₂—, and X is substituted or unsubstituted indolyl.

In another embodiment of the compounds of formula IH, M⁴ is —C(R⁴)₂—, R¹ is H, L² is —C(O)—, and X is substituted or unsubstituted indolyl.

In another embodiment of the compounds of formula IH, M⁴ is —CH₂—, R¹ is H, L² is —C(O)—, and X is substituted or unsubstituted indolyl.

In another embodiment of the compounds of formula IH, M⁴ is —CH(CH₃)—, R¹ is H, L² is —C(O)—, and X is substituted or unsubstituted indolyl.

In another embodiment of the compounds of formula IH, M⁴ is —C(R⁴)₂—, R¹ is alkyl, L² is —C(O)—, and X is substituted or unsubstituted indolyl.

In another embodiment of the compounds of formula IH, M⁴ is —CH₂—, R¹ is alkyl, L² is —C(O)—, and X is substituted or unsubstituted indolyl.

In another embodiment of the compounds of formula IH, M⁴ is —CH(CH₃)—, R¹ is alkyl, L² is —C(O)—, and X is substituted or unsubstituted indolyl.

In another embodiment of the compounds of formula IH, M⁴ is —C(R⁴)₂—, R¹ is substituted or unsubstituted arylalkyl, L² is —C(O)—, and X is substituted or unsubstituted indolyl.

In another embodiment of the compounds of formula IH, M⁴ is —CH₂—, R¹ is substituted or unsubstituted arylalkyl, L² is —C(O)—, and X is substituted or unsubstituted indolyl.

In another embodiment of the compounds of formula IH, M⁴ is —CH(CH₃)—, R¹ is substituted or unsubstituted arylalkyl, L² is —C(O)—, and X is substituted or unsubstituted indolyl.

In another embodiment of the compounds of formula IH, M⁴ is —C(R⁴)₂—, R¹ is H, L² is —S(O)₂—, and X is substituted or unsubstituted indolyl.

In another embodiment of the compounds of formula IH, M⁴ is —CH₂—, R¹ is H, L² is —S(O)₂—, and X is substituted or unsubstituted indolyl.

In another embodiment of the compounds of formula IH, M⁴ is —CH(CH₃)—, R¹ is H, L² is —S(O)₂—, and X is substituted or unsubstituted indolyl.

In another embodiment of the compounds of formula IH, M⁴ is —C(R⁴)₂—, R¹ is alkyl, L² is —S(O)₂—, and X is substituted or unsubstituted indolyl.

In another embodiment of the compounds of formula IH, M⁴ is —CH₂—, R¹ is alkyl, L² is —S(O)₂—, and X is substituted or unsubstituted indolyl.

In another embodiment of the compounds of formula IH, M⁴ is —CH(CH₃)—, R¹ is alkyl, L² is —S(O)₂—, and X is substituted or unsubstituted indolyl.

In another embodiment of the compounds of formula IH, M⁴ is —C(R⁴)₂—, R¹ is substituted or unsubstituted arylalkyl, L² is —S(O)₂—, and X is substituted or unsubstituted indolyl.

In another embodiment of the compounds of formula IH, M⁴ is —CH₂—, R¹ is substituted or unsubstituted arylalkyl, L² is —S(O)₂—, and X is substituted or unsubstituted indolyl.

In another embodiment of the compounds of formula IH, M⁴ is —CH(CH₃)—, R¹ is substituted or unsubstituted arylalkyl, L² is —S(O)₂—, and X is substituted or unsubstituted indolyl.

In another embodiment of the compounds of formula IH, M⁴ is —C(R⁴)₂—, R¹ is H, L² is a covalent bond, and X is substituted or unsubstituted benzimidazolyl.

In another embodiment of the compounds of formula IH, M⁴ is —CH₂—, R¹ is H, L² is a covalent bond, and X is substituted or unsubstituted benzimidazolyl.

In another embodiment of the compounds of formula IH, M⁴ is —CH(CH₃)—, R¹ is H, L² is a covalent bond, and X is substituted or unsubstituted benzimidazolyl.

In another embodiment of the compounds of formula IH, M⁴ is —C(R⁴)₂—, R¹ is alkyl, L² is a covalent bond, and X is substituted or unsubstituted benzimidazolyl.

In another embodiment of the compounds of formula IH, M⁴ is —CH₂—, R¹ is alkyl, L² is a covalent bond, and X is substituted or unsubstituted benzimidazolyl.

In another embodiment of the compounds of formula IH, M⁴ is —CH(CH₃)—, R¹ is alkyl, L² is a covalent bond, and X is substituted or unsubstituted benzimidazolyl.

In another embodiment of the compounds of formula IH, M⁴ is —C(R⁴)₂—, R¹ is substituted or unsubstituted arylalkyl, L² is a covalent bond, and X is substituted or unsubstituted benzimidazolyl.

In another embodiment of the compounds of formula IH, M⁴ is —CH₂—, R¹ is substituted or unsubstituted arylalkyl, L² is a covalent bond, and X is substituted or unsubstituted benzimidazolyl.

In another embodiment of the compounds of formula IH, M⁴ is —CH(CH₃)—, R¹ is substituted or unsubstituted arylalkyl, L² is a covalent bond, and X is substituted or unsubstituted benzimidazolyl.

In another embodiment of the compounds of formula IH, M⁴ is —C(R⁴)₂—, R¹ is H, L² is —C(R⁴)₂—, and X is substituted or unsubstituted benzimidazolyl.

In another embodiment of the compounds of formula IH, M⁴ is —CH₂—, R¹ is H, L² is —C(R⁴)₂—, and X is substituted or unsubstituted benzimidazolyl.

In another embodiment of the compounds of formula IH, M⁴ is —CH(CH₃)—, R¹ is H, L² is —C(R⁴)₂—, and X is substituted or unsubstituted benzimidazolyl.

In another embodiment of the compounds of formula IH, M⁴ is —C(R⁴)₂—, R¹ is alkyl, L² is —C(R⁴)₂—, and X is substituted or unsubstituted benzimidazolyl.

In another embodiment of the compounds of formula IH, M⁴ is —CH₂—, R¹ is alkyl, L² is —C(R⁴)₂—, and X is substituted or unsubstituted benzimidazolyl.

In another embodiment of the compounds of formula IH, M⁴ is —CH(CH₃)—, R¹ is alkyl, L² is —C(R⁴)₂—, and X is substituted or unsubstituted benzimidazolyl.

In another embodiment of the compounds of formula IH, M⁴ is —C(R⁴)₂—, R¹ is substituted or unsubstituted arylalkyl, L² is —C(R⁴)₂—, and X is substituted or unsubstituted benzimidazolyl.

In another embodiment of the compounds of formula IH, M⁴ is —CH₂—, R¹ is substituted or unsubstituted arylalkyl, L² is —C(R⁴)₂—, and X is substituted or unsubstituted benzimidazolyl.

In another embodiment of the compounds of formula IH, M⁴ is —CH(CH₃)—, R¹ is substituted or unsubstituted arylalkyl, L² is —C(R⁴)₂—, and X is substituted or unsubstituted benzimidazolyl.

In another embodiment of the compounds of formula IH, M⁴ is —C(R⁴)₂—, R¹ is H, L² is —C(O)—, and X is substituted or unsubstituted benzimidazolyl.

In another embodiment of the compounds of formula IH, M⁴ is —CH₂—, R¹ is H, L² is —C(O)—, and X is substituted or unsubstituted benzimidazolyl.

In another embodiment of the compounds of formula IH, M⁴ is —CH(CH₃)—, R¹ is H, L² is —C(O)—, and X is substituted or unsubstituted benzimidazolyl.

In another embodiment of the compounds of formula IH, M⁴ is —C(R⁴)₂—, R¹ is alkyl, L² is —C(O)—, and X is substituted or unsubstituted benzimidazolyl.

In another embodiment of the compounds of formula IH, M⁴ is —CH₂—, R¹ is alkyl, L² is —C(O)—, and X is substituted or unsubstituted benzimidazolyl.

In another embodiment of the compounds of formula IH, M⁴ is —CH(CH₃)—, R¹ is alkyl, L² is —C(O)—, and X is substituted or unsubstituted benzimidazolyl.

In another embodiment of the compounds of formula IH, M⁴ is —C(R⁴)₂—, R¹ is substituted or unsubstituted arylalkyl, L² is —C(O)—, and X is substituted or unsubstituted benzimidazolyl.

In another embodiment of the compounds of formula IH, M⁴ is —CH₂—, R¹ is substituted or unsubstituted arylalkyl, L² is —C(O)—, and X is substituted or unsubstituted benzimidazolyl.

In another embodiment of the compounds of formula IH, M⁴ is —CH(CH₃)—, R¹ is substituted or unsubstituted arylalkyl, L² is —C(O)—, and X is substituted or unsubstituted benzimidazolyl.

In another embodiment of the compounds of formula IH, M⁴ is —C(R⁴)₂—, R¹ is H, L² is —S(O)₂—, and X is substituted or unsubstituted benzimidazolyl.

In another embodiment of the compounds of formula IH, M⁴ is —CH₂—, R¹ is H, L² is —S(O)₂—, and X is substituted or unsubstituted benzimidazolyl.

In another embodiment of the compounds of formula IH, M⁴ is —CH(CH₃)—, R¹ is H, L² is —S(O)₂—, and X is substituted or unsubstituted benzimidazolyl.

In another embodiment of the compounds of formula IH, M⁴ is —C(R⁴)₂—, R¹ is alkyl, L² is —S(O)₂—, and X is substituted or unsubstituted benzimidazolyl.

In another embodiment of the compounds of formula IH, M⁴ is —CH₂—, R¹ is alkyl, L² is —S(O)₂—, and X is substituted or unsubstituted benzimidazolyl.

In another embodiment of the compounds of formula IH, M⁴ is —CH(CH₃)—, R¹ is alkyl, L² is —S(O)₂—, and X is substituted or unsubstituted benzimidazolyl.

In another embodiment of the compounds of formula IH, M⁴ is —C(R⁴)₂—, R¹ is substituted or unsubstituted arylalkyl, L² is —S(O)₂—, and X is substituted or unsubstituted benzimidazolyl.

In another embodiment of the compounds of formula IH, M⁴ is —CH₂—, R¹ is substituted or unsubstituted arylalkyl, L² is —S(O)₂—, and X is substituted or unsubstituted benzimidazolyl.

In another embodiment of the compounds of formula IH, M⁴ is —CH(CH₃)—, R¹ is substituted or unsubstituted arylalkyl, L² is —S(O)₂—, and X is substituted or unsubstituted benzimidazolyl.

In another embodiment of the compounds of formula IH, M⁴ is —C(R⁴)₂—, R¹ is H, L² is a covalent bond, and X is substituted or unsubstituted quinolyl.

In another embodiment of the compounds of formula IH, M⁴ is —CH₂—, R¹ is H, L² is a covalent bond, and X is substituted or unsubstituted quinolyl.

In another embodiment of the compounds of formula IH, M⁴ is —CH(CH₃)—, R¹ is H, L² is a covalent bond, and X is substituted or unsubstituted quinolyl.

In another embodiment of the compounds of formula IH, M⁴ is —C(R⁴)₂—, R¹ is alkyl, L² is a covalent bond, and X is substituted or unsubstituted quinolyl.

In another embodiment of the compounds of formula IH, M⁴ is —CH₂—, R¹ is alkyl, L² is a covalent bond, and X is substituted or unsubstituted quinolyl.

In another embodiment of the compounds of formula IH, M⁴ is —CH(CH₃)—, R¹ is alkyl, L² is a covalent bond, and X is substituted or unsubstituted quinolyl.

In another embodiment of the compounds of formula IH, M⁴ is —C(R⁴)₂—, R¹ is substituted or unsubstituted arylalkyl, L² is a covalent bond, and X is substituted or unsubstituted quinolyl.

In another embodiment of the compounds of formula IH, M⁴ is —CH₂—, R¹ is substituted or unsubstituted arylalkyl, L² is a covalent bond, and X is substituted or unsubstituted quinolyl.

In another embodiment of the compounds of formula IH, M⁴ is —CH(CH₃)—, R¹ is substituted or unsubstituted arylalkyl, L² is a covalent bond, and X is substituted or unsubstituted quinolyl.

In another embodiment of the compounds of formula IH, M⁴ is —C(R⁴)₂—, R¹ is H, L² is —C(R⁴)₂—, and X is substituted or unsubstituted quinolyl.

In another embodiment of the compounds of formula IH, M⁴ is —CH₂—, R¹ is H, L² is —C(R⁴)₂—, and X is substituted or unsubstituted quinolyl.

In another embodiment of the compounds of formula IH, M⁴ is —CH(CH₃)—, R¹ is H, L² is —C(R⁴)₂—, and X is substituted or unsubstituted quinolyl.

In another embodiment of the compounds of formula IH, M⁴ is —C(R⁴)₂—, R¹ is alkyl, L² is —C(R⁴)₂—, and X is substituted or unsubstituted quinolyl.

In another embodiment of the compounds of formula IH, M⁴ is —CH₂—, R¹ is alkyl, L² is —C(R⁴)₂—, and X is substituted or unsubstituted quinolyl.

In another embodiment of the compounds of formula IH, M⁴ is —CH(CH₃)—, R¹ is alkyl, L² is —C(R⁴)₂—, and X is substituted or unsubstituted quinolyl.

In another embodiment of the compounds of formula IH, M⁴ is —C(R⁴)₂—, R¹ is substituted or unsubstituted arylalkyl, L² is —C(R⁴)₂—, and X is substituted or unsubstituted quinolyl.

In another embodiment of the compounds of formula IH, M⁴ is —CH₂—, R¹ is substituted or unsubstituted arylalkyl, L² is —C(R⁴)₂—, and X is substituted or unsubstituted quinolyl.

In another embodiment of the compounds of formula IH, M⁴ is —CH(CH₃)—, R¹ is substituted or unsubstituted arylalkyl, L² is —C(R⁴)₂—, and X is substituted or unsubstituted quinolyl.

In another embodiment of the compounds of formula IH, M⁴ is —C(R⁴)₂—, R¹ is H, L² is —C(O)—, and X is substituted or unsubstituted quinolyl.

In another embodiment of the compounds of formula IH, M⁴ is —CH₂—, R¹ is H, L² is —C(O)—, and X is substituted or unsubstituted quinolyl.

In another embodiment of the compounds of formula IH, M⁴ is —CH(CH₃)—, R¹ is H, L² is —C(O)—, and X is substituted or unsubstituted quinolyl.

In another embodiment of the compounds of formula IH, M⁴ is —C(R⁴)₂—, R¹ is alkyl, L² is —C(O)—, and X is substituted or unsubstituted quinolyl.

In another embodiment of the compounds of formula IH, M⁴ is —CH₂—, R¹ is alkyl, L² is —C(O)—, and X is substituted or unsubstituted quinolyl.

In another embodiment of the compounds of formula IH, M⁴ is —CH(CH₃)—, R¹ is alkyl, L² is —C(O)—, and X is substituted or unsubstituted quinolyl.

In another embodiment of the compounds of formula IH, M⁴ is —C(R⁴)₂—, R¹ is substituted or unsubstituted arylalkyl, L² is —C(O)—, and X is substituted or unsubstituted quinolyl.

In another embodiment of the compounds of formula IH, M⁴ is —CH₂—, R¹ is substituted or unsubstituted arylalkyl, L² is —C(O)—, and X is substituted or unsubstituted quinolyl.

In another embodiment of the compounds of formula IH, M⁴ is —CH(CH₃)—, R¹ is substituted or unsubstituted arylalkyl, L² is —C(O)—, and X is substituted or unsubstituted quinolyl.

In another embodiment of the compounds of formula IH, M⁴ is —C(R⁴)₂—, R¹ is H, L² is —S(O)₂—, and X is substituted or unsubstituted quinolyl.

In another embodiment of the compounds of formula IH, M⁴ is —CH₂—, R¹ is H, L² is —S(O)₂—, and X is substituted or unsubstituted quinolyl.

In another embodiment of the compounds of formula IH, M⁴ is —CH(CH₃)—, R¹ is H, L² is —S(O)₂—, and X is substituted or unsubstituted quinolyl.

In another embodiment of the compounds of formula IH, M⁴ is —C(R⁴)₂—, R¹ is alkyl, L² is —S(O)₂—, and X is substituted or unsubstituted quinolyl.

In another embodiment of the compounds of formula IH, M⁴ is —CH₂—, R¹ is alkyl, L² is —S(O)₂—, and X is substituted or unsubstituted quinolyl.

In another embodiment of the compounds of formula IH, M⁴ is —CH(CH₃)—, R¹ is alkyl, L² is —S(O)₂—, and X is substituted or unsubstituted quinolyl.

In another embodiment of the compounds of formula IH, M⁴ is —C(R⁴)₂—, R¹ is substituted or unsubstituted arylalkyl, L² is —S(O)₂—, and X is substituted or unsubstituted quinolyl.

In another embodiment of the compounds of formula IH, M⁴ is —CH₂—, R¹ is substituted or unsubstituted arylalkyl, L² is —S(O)₂—, and X is substituted or unsubstituted quinolyl.

In another embodiment of the compounds of formula IH, M⁴ is —CH(CH₃)—, R¹ is substituted or unsubstituted arylalkyl, L² is —S(O)₂—, and X is substituted or unsubstituted quinolyl.

In another embodiment of the compounds of formula IH, M⁴ is —C(R⁴)₂—, R¹ is H, L² is a covalent bond, and X is substituted or unsubstituted thienyl.

In another embodiment of the compounds of formula IH, M⁴ is —CH₂—, R¹ is H, L² is a covalent bond, and X is substituted or unsubstituted thienyl.

In another embodiment of the compounds of formula IH, M⁴ is —CH(CH₃)—, R¹ is H, L² is a covalent bond, and X is substituted or unsubstituted thienyl.

In another embodiment of the compounds of formula IH, M⁴ is —C(R⁴)₂—, R¹ is alkyl, L² is a covalent bond, and X is substituted or unsubstituted thienyl.

In another embodiment of the compounds of formula IH, M⁴ is —CH₂—, R¹ is alkyl, L² is a covalent bond, and X is substituted or unsubstituted thienyl.

In another embodiment of the compounds of formula IH, M⁴ is —CH(CH₃)—, R¹ is alkyl, L² is a covalent bond, and X is substituted or unsubstituted thienyl.

In another embodiment of the compounds of formula IH, M⁴ is —C(R⁴)₂—, R¹ is substituted or unsubstituted arylalkyl, L² is a covalent bond, and X is substituted or unsubstituted thienyl.

In another embodiment of the compounds of formula IH, M⁴ is —CH₂—, R¹ is substituted or unsubstituted arylalkyl, L² is a covalent bond, and X is substituted or unsubstituted thienyl.

In another embodiment of the compounds of formula IH, M⁴ is —CH(CH₃)—, R¹ is substituted or unsubstituted arylalkyl, L² is a covalent bond, and X is substituted or unsubstituted thienyl.

In another embodiment of the compounds of formula IH, M⁴ is —C(R⁴)₂—, R¹ is H, L² is —C(R⁴)₂—, and X is substituted or unsubstituted thienyl.

In another embodiment of the compounds of formula IH, M⁴ is —CH₂—, R¹ is H, L² is —C(R⁴)₂—, and X is substituted or unsubstituted thienyl.

In another embodiment of the compounds of formula IH, M⁴ is —CH(CH₃)—, R¹ is H, L² is —C(R⁴)₂—, and X is substituted or unsubstituted thienyl.

In another embodiment of the compounds of formula IH, M⁴ is —C(R⁴)₂—, R¹ is alkyl, L² is —C(R⁴)₂—, and X is substituted or unsubstituted thienyl.

In another embodiment of the compounds of formula IH, M⁴ is —CH₂—, R¹ is alkyl, L² is —C(R⁴)₂—, and X is substituted or unsubstituted thienyl.

In another embodiment of the compounds of formula IH, M⁴ is —CH(CH₃)—, R¹ is alkyl, L² is —C(R⁴)₂—, and X is substituted or unsubstituted thienyl.

In another embodiment of the compounds of formula IH, M⁴ is —C(R⁴)₂—, R¹ is substituted or unsubstituted arylalkyl, L² is —C(R⁴)₂—, and X is substituted or unsubstituted thienyl.

In another embodiment of the compounds of formula IH, M⁴ is —CH₂—, R¹ is substituted or unsubstituted arylalkyl, L² is —C(R⁴)₂—, and X is substituted or unsubstituted thienyl.

In another embodiment of the compounds of formula IH, M⁴ is —CH(CH₃)—, R¹ is substituted or unsubstituted arylalkyl, L² is —C(R⁴)₂—, and X is substituted or unsubstituted thienyl.

In another embodiment of the compounds of formula IH, M⁴ is —C(R⁴)₂—, R¹ is H, L² is —C(O)—, and X is substituted or unsubstituted thienyl.

In another embodiment of the compounds of formula IH, M⁴ is —CH₂—, R¹ is H, L² is —C(O)—, and X is substituted or unsubstituted thienyl.

In another embodiment of the compounds of formula IH, M⁴ is —CH(CH₃)—, R¹ is H, L² is —C(O)—, and X is substituted or unsubstituted thienyl.

In another embodiment of the compounds of formula IH, M⁴ is —C(R⁴)₂—, R¹ is alkyl, L² is —C(O)—, and X is substituted or unsubstituted thienyl.

In another embodiment of the compounds of formula IH, M⁴ is —CH₂—, R¹ is alkyl, L² is —C(O)—, and X is substituted or unsubstituted thienyl.

In another embodiment of the compounds of formula IH, M⁴ is —CH(CH₃)—, R¹ is alkyl, L² is —C(O)—, and X is substituted or unsubstituted thienyl.

In another embodiment of the compounds of formula IH, M⁴ is —C(R⁴)₂—, R¹ is substituted or unsubstituted arylalkyl, L² is —C(O)—, and X is substituted or unsubstituted thienyl.

In another embodiment of the compounds of formula IH, M⁴ is —CH₂—, R¹ is substituted or unsubstituted arylalkyl, L² is —C(O)—, and X is substituted or unsubstituted thienyl.

In another embodiment of the compounds of formula IH, M⁴ is —CH(CH₃)—, R¹ is substituted or unsubstituted arylalkyl, L² is —C(O)—, and X is substituted or unsubstituted thienyl.

In another embodiment of the compounds of formula IH, M⁴ is —C(R⁴)₂—, R¹ is H, L² is —S(O)₂—, and X is substituted or unsubstituted thienyl.

In another embodiment of the compounds of formula IH, M⁴ is —CH₂—, R¹ is H, L² is —S(O)₂—, and X is substituted or unsubstituted thienyl.

In another embodiment of the compounds of formula IH, M⁴ is —CH(CH₃)—, R¹ is H, L² is —S(O)₂—, and X is substituted or unsubstituted thienyl.

In another embodiment of the compounds of formula IH, M⁴ is —C(R⁴)₂—, R¹ is alkyl, L² is —S(O)₂—, and X is substituted or unsubstituted thienyl.

In another embodiment of the compounds of formula IH, M⁴ is —CH₂—, R¹ is alkyl, L² is —S(O)₂—, and X is substituted or unsubstituted thienyl.

In another embodiment of the compounds of formula IH, M⁴ is —CH(CH₃)—, R¹ is alkyl, L² is —S(O)₂—, and X is substituted or unsubstituted thienyl.

In another embodiment of the compounds of formula IH, M⁴ is —C(R⁴)₂—, R¹ is substituted or unsubstituted arylalkyl, L² is —S(O)₂—, and X is substituted or unsubstituted thienyl.

In another embodiment of the compounds of formula IH, M⁴ is —CH₂—, R¹ is substituted or unsubstituted arylalkyl, L² is —S(O)₂—, and X is substituted or unsubstituted thienyl.

In another embodiment of the compounds of formula IH, M⁴ is —CH(CH₃)—, R¹ is substituted or unsubstituted arylalkyl, L² is —S(O)₂—, and X is substituted or unsubstituted thienyl.

In another embodiment of the compounds of formula IH, M⁴ is —C(R⁴)₂—, R¹ is H, L² is a covalent bond, and X is substituted or unsubstituted furanyl.

In another embodiment of the compounds of formula IH, M⁴ is —CH₂—, R¹ is H, L² is a covalent bond, and X is substituted or unsubstituted furanyl.

In another embodiment of the compounds of formula IH, M⁴ is —CH(CH₃)—, R¹ is H, L² is a covalent bond, and X is substituted or unsubstituted furanyl.

In another embodiment of the compounds of formula IH, M⁴ is —C(R⁴)₂—, R¹ is alkyl, L² is a covalent bond, and X is substituted or unsubstituted furanyl.

In another embodiment of the compounds of formula IH, M⁴ is —CH₂—, R¹ is alkyl, L² is a covalent bond, and X is substituted or unsubstituted furanyl.

In another embodiment of the compounds of formula IH, M⁴ is —CH(CH₃)—, R¹ is alkyl, L² is a covalent bond, and X is substituted or unsubstituted furanyl.

In another embodiment of the compounds of formula IH, M⁴ is —C(R⁴)₂—, R¹ is substituted or unsubstituted arylalkyl, L² is a covalent bond, and X is substituted or unsubstituted furanyl.

In another embodiment of the compounds of formula IH, M⁴ is —CH₂—, R¹ is substituted or unsubstituted arylalkyl, L² is a covalent bond, and X is substituted or unsubstituted furanyl.

In another embodiment of the compounds of formula IH, M⁴ is —CH(CH₃)—, R¹ is substituted or unsubstituted arylalkyl, L² is a covalent bond, and X is substituted or unsubstituted furanyl.

In another embodiment of the compounds of formula IH, M⁴ is —C(R⁴)₂—, R¹ is H, L² is —C(R⁴)₂—, and X is substituted or unsubstituted furanyl.

In another embodiment of the compounds of formula IH, M⁴ is —CH₂—, R¹ is H, L² is —C(R⁴)₂—, and X is substituted or unsubstituted furanyl.

In another embodiment of the compounds of formula IH, M⁴ is —CH(CH₃)—, R¹ is H, L² is —C(R⁴)₂—, and X is substituted or unsubstituted furanyl.

In another embodiment of the compounds of formula IH, M⁴ is —C(R⁴)₂—, R¹ is alkyl, L² is —C(R⁴)₂—, and X is substituted or unsubstituted furanyl.

In another embodiment of the compounds of formula IH, M⁴ is —CH₂—, R¹ is alkyl, L² is —C(R⁴)₂—, and X is substituted or unsubstituted furanyl.

In another embodiment of the compounds of formula IH, M⁴ is —CH(CH₃)—, R¹ is alkyl, L² is —C(R⁴)₂—, and X is substituted or unsubstituted furanyl.

In another embodiment of the compounds of formula IH, M⁴ is —C(R⁴)₂—, R¹ is substituted or unsubstituted arylalkyl, L² is —C(R⁴)₂—, and X is substituted or unsubstituted furanyl.

In another embodiment of the compounds of formula IH, M⁴ is —CH₂—, R¹ is substituted or unsubstituted arylalkyl, L² is —C(R⁴)₂—, and X is substituted or unsubstituted furanyl.

In another embodiment of the compounds of formula IH, M⁴ is —CH(CH₃)—, R¹ is substituted or unsubstituted arylalkyl, L² is —C(R⁴)₂—, and X is substituted or unsubstituted furanyl.

In another embodiment of the compounds of formula IH, M⁴ is —C(R⁴)₂—, R¹ is H, L² is —C(O)—, and X is substituted or unsubstituted furanyl.

In another embodiment of the compounds of formula IH, M⁴ is —CH₂—, R¹ is H, L² is —C(O)—, and X is substituted or unsubstituted furanyl.

In another embodiment of the compounds of formula IH, M⁴ is —CH(CH₃)—, R¹ is H, L² is —C(O)—, and X is substituted or unsubstituted furanyl.

In another embodiment of the compounds of formula IH, M⁴ is —C(R⁴)₂—, R¹ is alkyl, L² is —C(O)—, and X is substituted or unsubstituted furanyl.

In another embodiment of the compounds of formula IH, M⁴ is —CH₂—, R¹ is alkyl, L² is —C(O)—, and X is substituted or unsubstituted furanyl.

In another embodiment of the compounds of formula IH, M⁴ is —CH(CH₃)—, R¹ is alkyl, L² is —C(O)—, and X is substituted or unsubstituted furanyl.

In another embodiment of the compounds of formula IH, M⁴ is —C(R⁴)₂—, R¹ is substituted or unsubstituted arylalkyl, L² is —C(O)—, and X is substituted or unsubstituted furanyl.

In another embodiment of the compounds of formula IH, M⁴ is —CH₂—, R¹ is substituted or unsubstituted arylalkyl, L² is —C(O)—, and X is substituted or unsubstituted furanyl.

In another embodiment of the compounds of formula IH, M⁴ is —CH(CH₃)—, R¹ is substituted or unsubstituted arylalkyl, L² is —C(O)—, and X is substituted or unsubstituted furanyl.

In another embodiment of the compounds of formula IH, M⁴ is —C(R⁴)₂—, R¹ is H, L² is —S(O)₂—, and X is substituted or unsubstituted furanyl.

In another embodiment of the compounds of formula IH, M⁴ is —CH₂—, R¹ is H, L² is —S(O)₂—, and X is substituted or unsubstituted furanyl.

In another embodiment of the compounds of formula IH, M⁴ is —CH(CH₃)—, R¹ is H, L² is —S(O)₂—, and X is substituted or unsubstituted furanyl.

In another embodiment of the compounds of formula IH, M⁴ is —C(R⁴)₂—, R¹ is alkyl, L² is —S(O)₂—, and X is substituted or unsubstituted furanyl.

In another embodiment of the compounds of formula IH, M⁴ is —CH₂—, R¹ is alkyl, L² is —S(O)₂—, and X is substituted or unsubstituted furanyl.

In another embodiment of the compounds of formula IH, M⁴ is —CH(CH₃)—, R¹ is alkyl, L² is —S(O)₂—, and X is substituted or unsubstituted furanyl.

In another embodiment of the compounds of formula IH, M⁴ is —C(R⁴)₂—, R¹ is substituted or unsubstituted arylalkyl, L² is —S(O)₂—, and X is substituted or unsubstituted furanyl.

In another embodiment of the compounds of formula IH, M⁴ is —CH₂—, R¹ is substituted or unsubstituted arylalkyl, L² is —S(O)₂—, and X is substituted or unsubstituted furanyl.

In another embodiment of the compounds of formula IH, M⁴ is —CH(CH₃)—, R¹ is substituted or unsubstituted arylalkyl, L² is —S(O)₂—, and X is substituted or unsubstituted furanyl.

In another embodiment of the compounds of formula IH, M⁴ is —C(R⁴)₂—, R¹ is H, L² is a covalent bond, and X is substituted or unsubstituted pyrrolyl.

In another embodiment of the compounds of formula IH, M⁴ is —CH₂—, R¹ is H, L² is a covalent bond, and X is substituted or unsubstituted pyrrolyl.

In another embodiment of the compounds of formula IH, M⁴ is —CH(CH₃)—, R¹ is H, L² is a covalent bond, and X is substituted or unsubstituted pyrrolyl.

In another embodiment of the compounds of formula IH, M⁴ is —C(R⁴)₂—, R¹ is alkyl, L² is a covalent bond, and X is substituted or unsubstituted pyrrolyl.

In another embodiment of the compounds of formula IH, M⁴ is —CH₂—, R¹ is alkyl, L² is a covalent bond, and X is substituted or unsubstituted pyrrolyl.

In another embodiment of the compounds of formula IH, M⁴ is —CH(CH₃)—, R¹ is alkyl, L² is a covalent bond, and X is substituted or unsubstituted pyrrolyl.

In another embodiment of the compounds of formula IH, M⁴ is —C(R⁴)₂—, R¹ is substituted or unsubstituted arylalkyl, L² is a covalent bond, and X is substituted or unsubstituted pyrrolyl.

In another embodiment of the compounds of formula IH, M⁴ is —CH₂—, R¹ is substituted or unsubstituted arylalkyl, L² is a covalent bond, and X is substituted or unsubstituted pyrrolyl.

In another embodiment of the compounds of formula IH, M⁴ is —CH(CH₃)—, R¹ is substituted or unsubstituted arylalkyl, L² is a covalent bond, and X is substituted or unsubstituted pyrrolyl.

In another embodiment of the compounds of formula IH, M⁴ is —C(R⁴)₂—, R¹ is H, L² is —C(R⁴)₂—, and X is substituted or unsubstituted pyrrolyl.

In another embodiment of the compounds of formula IH, M⁴ is —CH₂—, R¹ is H, L² is —C(R⁴)₂—, and X is substituted or unsubstituted pyrrolyl.

In another embodiment of the compounds of formula IH, M⁴ is —CH(CH₃)—, R¹ is H, L² is —C(R⁴)₂—, and X is substituted or unsubstituted pyrrolyl.

In another embodiment of the compounds of formula IH, M⁴ is —C(R⁴)₂—, R¹ is alkyl, L² is —C(R⁴)₂—, and X is substituted or unsubstituted pyrrolyl.

In another embodiment of the compounds of formula IH, M⁴ is —CH₂—, R¹ is alkyl, L² is —C(R⁴)₂—, and X is substituted or unsubstituted pyrrolyl.

In another embodiment of the compounds of formula IH, M⁴ is —CH(CH₃)—, R¹ is alkyl, L² is —C(R⁴)₂—, and X is substituted or unsubstituted pyrrolyl.

In another embodiment of the compounds of formula IH, M⁴ is —C(R⁴)₂—, R¹ is substituted or unsubstituted arylalkyl, L² is —C(R⁴)₂—, and X is substituted or unsubstituted pyrrolyl.

In another embodiment of the compounds of formula IH, M⁴ is —CH₂—, R¹ is substituted or unsubstituted arylalkyl, L² is —C(R⁴)₂—, and X is substituted or unsubstituted pyrrolyl.

In another embodiment of the compounds of formula IH, M⁴ is —CH(CH₃)—, R¹ is substituted or unsubstituted arylalkyl, L² is —C(R⁴)₂—, and X is substituted or unsubstituted pyrrolyl.

In another embodiment of the compounds of formula IH, M⁴ is —C(R⁴)₂—, R¹ is H, L² is —C(O)—, and X is substituted or unsubstituted pyrrolyl.

In another embodiment of the compounds of formula IH, M⁴ is —CH₂—, R¹ is H, L² is —C(O)—, and X is substituted or unsubstituted pyrrolyl.

In another embodiment of the compounds of formula IH, M⁴ is —CH(CH₃)—, R¹ is H, L² is —C(O)—, and X is substituted or unsubstituted pyrrolyl.

In another embodiment of the compounds of formula IH, M⁴ is —C(R⁴)₂—, R¹ is alkyl, L² is —C(O)—, and X is substituted or unsubstituted pyrrolyl.

In another embodiment of the compounds of formula IH, M⁴ is —CH₂—, R¹ is alkyl, L² is —C(O)—, and X is substituted or unsubstituted pyrrolyl.

In another embodiment of the compounds of formula IH, M⁴ is —CH(CH₃)—, R¹ is alkyl, L² is —C(O)—, and X is substituted or unsubstituted pyrrolyl.

In another embodiment of the compounds of formula IH, M⁴ is —C(R⁴)₂—, R¹ is substituted or unsubstituted arylalkyl, L² is —C(O)—, and X is substituted or unsubstituted pyrrolyl.

In another embodiment of the compounds of formula IH, M⁴ is —CH₂—, R¹ is substituted or unsubstituted arylalkyl, L² is —C(O)—, and X is substituted or unsubstituted pyrrolyl.

In another embodiment of the compounds of formula IH, M⁴ is —CH(CH₃)—, R¹ is substituted or unsubstituted arylalkyl, L² is —C(O)—, and X is substituted or unsubstituted pyrrolyl.

In another embodiment of the compounds of formula IH, M⁴ is —C(R⁴)₂—, R¹ is H, L² is —S(O)₂—, and X is substituted or unsubstituted pyrrolyl.

In another embodiment of the compounds of formula IH, M⁴ is —CH₂—, R¹ is H, L² is —S(O)₂—, and X is substituted or unsubstituted pyrrolyl.

In another embodiment of the compounds of formula IH, M⁴ is —CH(CH₃)—, R¹ is H, L² is —S(O)₂—, and X is substituted or unsubstituted pyrrolyl.

In another embodiment of the compounds of formula IH, M⁴ is —C(R⁴)₂—, R¹ is alkyl, L² is —S(O)₂—, and X is substituted or unsubstituted pyrrolyl.

In another embodiment of the compounds of formula IH, M⁴ is —CH₂—, R¹ is alkyl, L² is —S(O)₂—, and X is substituted or unsubstituted pyrrolyl.

In another embodiment of the compounds of formula IH, M⁴ is —CH(CH₃)—, R¹ is alkyl, L² is —S(O)₂—, and X is substituted or unsubstituted pyrrolyl.

In another embodiment of the compounds of formula IH, M⁴ is —C(R⁴)₂—, R¹ is substituted or unsubstituted arylalkyl, L² is —S(O)₂—, and X is substituted or unsubstituted pyrrolyl.

In another embodiment of the compounds of formula IH, M⁴ is —CH₂—, R¹ is substituted or unsubstituted arylalkyl, L² is —S(O)₂—, and X is substituted or unsubstituted pyrrolyl.

In another embodiment of the compounds of formula IH, M⁴ is —CH(CH₃)—, R¹ is substituted or unsubstituted arylalkyl, L² is —S(O)₂—, and X is substituted or unsubstituted pyrrolyl.

In another embodiment of the compounds of formula IH, M⁴ is —C(R⁴)₂—, and L⁵ is —C(R⁴)₂—.

In another embodiment of the compounds of formula IH, M⁴ is —CH₂—, and L⁵ is —C(R⁴)₂—.

In another embodiment of the compounds of formula IH, M⁴ is —CH(CH₃)—, and L⁵ is —C(R⁴)₂—.

In another embodiment of the compounds of formula IH, M⁴ is —C(R⁴)₂—, and L⁵ is —CH₂—.

In another embodiment of the compounds of formula IH, M⁴ is —CH₂—, and L⁵ is —CH₂—

In another embodiment of the compounds of formula IH, M⁴ is —CH(CH₃)—, and L⁵ is —CH₂—.

In another embodiment of the compounds of formula IH, M⁴ is —C(R⁴)₂—, and L⁵ is —CH(CH₃)—.

In another embodiment of the compounds of formula IH, M⁴ is —CH₂—, and L⁵ is —CH(CH₃)—.

In another embodiment of the compounds of formula IH, M⁴ is —CH(CH₃)—, and L⁵ is —CH(CH₃)—.

In another embodiment of the compounds of formula IH, M⁴ is —C(R⁴)₂—, L⁵ is —C(R⁴)₂—, and Z is substituted or unsubstituted aryl.

In another embodiment of the compounds of formula IH, M⁴ is —CH₂—, L⁵ is —C(R⁴)₂—, and Z is substituted or unsubstituted aryl.

In another embodiment of the compounds of formula IH, M⁴ is —CH(CH₃)—, L⁵ is —C(R⁴)₂—, and Z is substituted or unsubstituted aryl.

In another embodiment of the compounds of formula IH, M⁴ is —C(R⁴)₂—, L⁵ is —CH₂—, and Z is substituted or unsubstituted aryl.

In another embodiment of the compounds of formula IH, M⁴ is —CH₂—, L⁵ is —CH₂—, and Z is substituted or unsubstituted aryl.

In another embodiment of the compounds of formula IH, M⁴ is —CH(CH₃)—, L⁵ is —CH₂—, and Z is substituted or unsubstituted aryl.

In another embodiment of the compounds of formula IH, M⁴ is —C(R⁴)₂—, L⁵ is —CH(CH₃)—, and Z is substituted or unsubstituted aryl.

In another embodiment of the compounds of formula IH, M⁴ is —CH₂—, L⁵ is —CH(CH₃)—, and Z is substituted or unsubstituted aryl.

In another embodiment of the compounds of formula IH, M⁴ is —CH(CH₃)—, L⁵ is —CH(CH₃)—, and Z is substituted or unsubstituted aryl.

In another embodiment of the compounds of formula IH, M⁴ is —C(R⁴)₂—, L¹ is —C(R⁴)₂—, and Z is substituted or unsubstituted phenyl.

In another embodiment of the compounds of formula IH, M⁴ is —CH₂—, L⁵ is —C(R⁴)₂—, and Z is substituted or unsubstituted phenyl.

In another embodiment of the compounds of formula IH, M⁴ is —CH(CH₃)—, L⁵ is —C(R⁴)₂—, and Z is substituted or unsubstituted phenyl.

In another embodiment of the compounds of formula IH, M⁴ is —C(R⁴)₂—, L¹ is —CH₂—, and Z is substituted or unsubstituted phenyl.

In another embodiment of the compounds of formula IH, M⁴ is —CH₂—, L⁵ is —CH₂—, and Z is substituted or unsubstituted phenyl.

In another embodiment of the compounds of formula IH, M⁴ is —CH(CH₃)—, L⁵ is —CH₂—, and Z is substituted or unsubstituted phenyl.

In another embodiment of the compounds of formula IH, M⁴ is —C(R⁴)₂—, L⁵ is —CH(CH₃)—, and Z is substituted or unsubstituted phenyl.

In another embodiment of the compounds of formula IH, M⁴ is —CH₂—, L⁵ is —CH(CH₃)—, and Z is substituted or unsubstituted phenyl.

In another embodiment of the compounds of formula IH, M⁴ is —CH(CH₃)—, L⁵ is —CH(CH₃)—, and Z is substituted or unsubstituted phenyl.

In another embodiment of the compounds of formula IH, M⁴ is —C(R⁴)₂—, L⁵ is —C(R⁴)₂—, and Z is substituted or unsubstituted heteroaryl.

In another embodiment of the compounds of formula IH, M⁴ is —CH₂—, L⁵ is —C(R⁴)₂—, and Z is substituted or unsubstituted heteroaryl.

In another embodiment of the compounds of formula IH, M⁴ is —CH(CH₃)—, L⁵ is —C(R⁴)₂—, and Z is substituted or unsubstituted heteroaryl.

In another embodiment of the compounds of formula IH, M⁴ is —C(R⁴)₂—, L⁵ is —CH₂—, and Z is substituted or unsubstituted heteroaryl.

In another embodiment of the compounds of formula IH, M⁴ is —CH₂—, L₅ is —CH₂—, and Z is substituted or unsubstituted heteroaryl.

In another embodiment of the compounds of formula IH, M⁴ is —CH(CH₃)—, L⁵ is —CH₂—, and Z is substituted or unsubstituted heteroaryl.

In another embodiment of the compounds of formula IH, M⁴ is —C(R⁴)₂—, L⁵ is —CH(CH₃)—, and Z is substituted or unsubstituted heteroaryl.

In another embodiment of the compounds of formula IH, M⁴ is —CH₂—, L⁵ is —CH(CH₃)—, and Z is substituted or unsubstituted heteroaryl.

In another embodiment of the compounds of formula IH, M⁴ is —CH(CH₃)—, L⁵ is —CH(CH₃)—, and Z is substituted or unsubstituted heteroaryl.

In another embodiment of the compounds of formula IH, M⁴ is —C(R⁴)₂—, L⁵ is —C(R⁴)₂—, and Z is substituted or unsubstituted pyridyl.

In another embodiment of the compounds of formula IH, M⁴ is —CH₂—, L⁵ is —C(R⁴)₂—, and Z is substituted or unsubstituted pyridyl.

In another embodiment of the compounds of formula IH, M⁴ is —CH(CH₃)—, L⁵ is —C(R⁴)₂—, and Z is substituted or unsubstituted pyridyl.

In another embodiment of the compounds of formula IH, M⁴ is —C(R⁴)₂—, L⁵ is —CH₂—, and Z is substituted or unsubstituted pyridyl.

In another embodiment of the compounds of formula IH, M⁴ is —CH₂—, L⁵ is —CH₂—, and Z is substituted or unsubstituted pyridyl.

In another embodiment of the compounds of formula IH, M⁴ is —CH(CH₃)—, L⁵ is —CH₂—, and Z is substituted or unsubstituted pyridyl.

In another embodiment of the compounds of formula IH, M⁴ is —C(R⁴)₂—, L⁵ is —CH(CH₃)—, and Z is substituted or unsubstituted pyridyl.

In another embodiment of the compounds of formula IH, M⁴ is —CH₂—, L⁵ is —CH(CH₃)—, and Z is substituted or unsubstituted pyridyl.

In another embodiment of the compounds of formula IH, M⁴ is —CH(CH₃)—, L⁵ is —CH(CH₃)—, and Z is substituted or unsubstituted pyridyl.

In another embodiment of the compounds of formula IH, M⁴ is —C(R⁴)₂—, L⁵ is —C(R⁴)₂—, and Z is substituted or unsubstituted pyrimidyl.

In another embodiment of the compounds of formula IH, M⁴ is —CH₂—, L⁵ is —C(R⁴)₂—, and Z is substituted or unsubstituted pyrimidyl.

In another embodiment of the compounds of formula IH, M⁴ is —CH(CH₃)—, L⁵ is —C(R⁴)₂—, and Z is substituted or unsubstituted pyrimidyl.

In another embodiment of the compounds of formula IH, M⁴ is —C(R⁴)₂—, L⁵ is —CH₂—, and Z is substituted or unsubstituted pyrimidyl.

In another embodiment of the compounds of formula IH, M⁴ is —CH₂—, L⁵ is —CH₂—, and Z is substituted or unsubstituted pyrimidyl.

In another embodiment of the compounds of formula IH, M⁴ is —CH(CH₃)—, L⁵ is —CH₂—, and Z is substituted or unsubstituted pyrimidyl.

In another embodiment of the compounds of formula IH, M⁴ is —C(R⁴)₂—, L⁵ is —CH(CH₃)—, and Z is substituted or unsubstituted pyrimidyl.

In another embodiment of the compounds of formula IH, M⁴ is —CH₂—, L⁵ is —CH(CH₃)—, and Z is substituted or unsubstituted pyrimidyl.

In another embodiment of the compounds of formula IH, M⁴ is —CH(CH₃)—, L⁵ is —CH(CH₃)—, and Z is substituted or unsubstituted pyrimidyl.

In another embodiment of the compounds of formula IH, M⁴ is —C(R⁴)₂—, L⁵ is —C(R⁴)₂—, and Z is substituted or unsubstituted indolyl.

In another embodiment of the compounds of formula IH, M⁴ is —CH₂—, L⁵ is —C(R⁴)₂—, and Z is substituted or unsubstituted indolyl.

In another embodiment of the compounds of formula IH, M⁴ is —CH(CH₃)—, L⁵ is —C(R⁴)₂—, and Z is substituted or unsubstituted indolyl.

In another embodiment of the compounds of formula IH, M⁴ is —C(R⁴)₂—, L⁵ is —CH₂—, and Z is substituted or unsubstituted indolyl.

In another embodiment of the compounds of formula IH, M⁴ is —CH₂—, L⁵ is —CH₂—, and Z is substituted or unsubstituted indolyl.

In another embodiment of the compounds of formula IH, M⁴ is —CH(CH₃)—, L⁵ is —CH₂—, and Z is substituted or unsubstituted indolyl.

In another embodiment of the compounds of formula IH, M⁴ is —C(R⁴)₂—, L⁵ is —CH(CH₃)—, and Z is substituted or unsubstituted indolyl.

In another embodiment of the compounds of formula IH, M⁴ is —CH₂—, L⁵ is —CH(CH₃)—, and Z is substituted or unsubstituted indolyl.

In another embodiment of the compounds of formula IH, M⁴ is —CH(CH₃)—, L⁵ is —CH(CH₃)—, and Z is substituted or unsubstituted indolyl.

In another embodiment of the compounds of formula IH, M⁴ is —C(R⁴)₂—, L⁵ is —C(R⁴)₂—, and Z is substituted or unsubstituted quinolyl.

In another embodiment of the compounds of formula IH, M⁴ is —CH₂—, L⁵ is —C(R⁴)₂—, and Z is substituted or unsubstituted quinolyl.

In another embodiment of the compounds of formula IH, M⁴ is —CH(CH₃)—, L⁵ is —C(R⁴)₂—, and Z is substituted or unsubstituted quinolyl.

In another embodiment of the compounds of formula IH, M⁴ is —C(R⁴)₂—, L⁵ is —CH₂—, and Z is substituted or unsubstituted quinolyl.

In another embodiment of the compounds of formula IH, M⁴ is —CH₂—, L⁵ is —CH₂—, and Z is substituted or unsubstituted quinolyl.

In another embodiment of the compounds of formula IH, M⁴ is —CH(CH₃)—, L⁵ is —CH₂—, and Z is substituted or unsubstituted quinolyl.

In another embodiment of the compounds of formula IH, M⁴ is —C(R⁴)₂—, L⁵ is —CH(CH₃)—, and Z is substituted or unsubstituted quinolyl.

In another embodiment of the compounds of formula IH, M⁴ is —CH₂—, L⁵ is —CH(CH₃)—, and Z is substituted or unsubstituted quinolyl.

In another embodiment of the compounds of formula IH, M⁴ is —CH(CH₃)—, L⁵ is —CH(CH₃)—, and Z is substituted or unsubstituted quinolyl.

In another embodiment of the compounds of formula IH, M⁴ is —C(R⁴)₂—, L⁵ is —C(R⁴)₂—, and Z is substituted or unsubstituted benzimidazolyl.

In another embodiment of the compounds of formula IH, M⁴ is —CH₂—, L⁵ is —C(R⁴)₂—, and Z is substituted or unsubstituted benzimidazolyl.

In another embodiment of the compounds of formula IH, M⁴ is —CH(CH₃)—, L⁵ is —C(R⁴)₂—, and Z is substituted or unsubstituted benzimidazolyl.

In another embodiment of the compounds of formula IH, M⁴ is —C(R⁴)₂—, L⁵ is —CH₂—, and Z is substituted or unsubstituted benzimidazolyl.

In another embodiment of the compounds of formula IH, M⁴ is —CH₂—, L⁵ is —CH₂—, and Z is substituted or unsubstituted benzimidazolyl.

In another embodiment of the compounds of formula IH, M⁴ is —CH(CH₃)—, L⁵ is —CH₂—, and Z is substituted or unsubstituted benzimidazolyl.

In another embodiment of the compounds of formula IH, M⁴ is —C(R⁴)₂—, L⁵ is —CH(CH₃)—, and Z is substituted or unsubstituted benzimidazolyl.

In another embodiment of the compounds of formula IH, M⁴ is —CH₂—, L⁵ is —CH(CH₃)—, and Z is substituted or unsubstituted benzimidazolyl.

In another embodiment of the compounds of formula IH, M⁴ is —CH(CH₃)—, L⁵ is —CH(CH₃)—, and Z is substituted or unsubstituted benzimidazolyl.

In another embodiment of the compounds of formula IH, M⁴ is —C(R⁴)₂—, L⁵ is —C(R⁴)₂—, and Z is substituted or unsubstituted purinyl.

In another embodiment of the compounds of formula IH, M⁴ is —CH₂—, L⁵ is —C(R⁴)₂—, and Z is substituted or unsubstituted purinyl.

In another embodiment of the compounds of formula IH, M⁴ is —CH(CH₃)—, L⁵ is —C(R⁴)₂—, and Z is substituted or unsubstituted purinyl.

In another embodiment of the compounds of formula IH, M⁴ is —C(R⁴)₂—, L⁵ is —CH₂—, and Z is substituted or unsubstituted purinyl.

In another embodiment of the compounds of formula IH, M⁴ is —CH₂—, L⁵ is —CH₂—, and Z is substituted or unsubstituted purinyl.

In another embodiment of the compounds of formula IH, M⁴ is —CH(CH₃)—, L⁵ is —CH₂—, and Z is substituted or unsubstituted purinyl.

In another embodiment of the compounds of formula IH, M⁴ is —C(R⁴)₂—, L⁵ is —CH(CH₃)—, and Z is substituted or unsubstituted purinyl.

In another embodiment of the compounds of formula IH, M⁴ is —CH₂—, L⁵ is —CH(CH₃)—, and Z is substituted or unsubstituted purinyl.

In another embodiment of the compounds of formula IH, M⁴ is —CH(CH₃)—, L⁵ is —CH(CH₃)—, and Z is substituted or unsubstituted purinyl.

In another embodiment of the compounds of formula IH, M⁴ is —C(R⁴)₂—, L⁵ is —C(R⁴)₂—, and Z is substituted or unsubstituted thienyl.

In another embodiment of the compounds of formula IH, M⁴ is —CH₂—, L⁵ is —C(R⁴)₂—, and Z is substituted or unsubstituted thienyl.

In another embodiment of the compounds of formula IH, M⁴ is —CH(CH₃)—, L⁵ is —C(R⁴)₂—, and Z is substituted or unsubstituted thienyl.

In another embodiment of the compounds of formula IH, M⁴ is —C(R⁴)₂—, L⁵ is —CH₂—, and Z is substituted or unsubstituted thienyl.

In another embodiment of the compounds of formula IH, M⁴ is —CH₂—, L⁵ is —CH₂—, and Z is substituted or unsubstituted thienyl.

In another embodiment of the compounds of formula IH, M⁴ is —CH(CH₃)—, L⁵ is —CH₂—, and Z is substituted or unsubstituted thienyl.

In another embodiment of the compounds of formula IH, M⁴ is —C(R⁴)₂—, L⁵ is —CH(CH₃)—, and Z is substituted or unsubstituted thienyl.

In another embodiment of the compounds of formula IH, M⁴ is —CH₂—, L⁵ is —CH(CH₃)—, and Z is substituted or unsubstituted thienyl.

In another embodiment of the compounds of formula IH, M⁴ is —CH(CH₃)—, L⁵ is —CH(CH₃)—, and Z is substituted or unsubstituted thienyl.

In another embodiment of the compounds of formula IH, M⁴ is —C(R⁴)₂—, L⁵ is —C(R⁴)₂—, and Z is substituted or unsubstituted furanyl.

In another embodiment of the compounds of formula IH, M⁴ is —CH₂—, L⁵ is —C(R⁴)₂—, and Z is substituted or unsubstituted furanyl.

In another embodiment of the compounds of formula IH, M⁴ is —CH(CH₃)—, L⁵ is —C(R⁴)₂—, and Z is substituted or unsubstituted furanyl.

In another embodiment of the compounds of formula IH, M⁴ is —C(R⁴)₂—, L⁵ is —CH₂—, and Z is substituted or unsubstituted furanyl.

In another embodiment of the compounds of formula IH, M⁴ is —CH₂—, L⁵ is —CH₂—, and Z is substituted or unsubstituted furanyl.

In another embodiment of the compounds of formula IH, M⁴ is —CH(CH₃)—, L⁵ is —CH₂—, and Z is substituted or unsubstituted furanyl.

In another embodiment of the compounds of formula IH, M⁴ is —C(R⁴)₂—, L¹ is —CH(CH₃)—, and Z is substituted or unsubstituted furanyl.

In another embodiment of the compounds of formula IH, M⁴ is —CH₂—, L⁵ is —CH(CH₃)—, and Z is substituted or unsubstituted furanyl.

In another embodiment of the compounds of formula IH, M⁴ is —CH(CH₃)—, L⁵ is —CH(CH₃)—, and Z is substituted or unsubstituted furanyl.

In another embodiment of the compounds of formula IH, M⁴ is —C(R⁴)₂—, L⁵ is —C(R⁴)₂—, and Z is substituted or unsubstituted pyrrolyl.

In another embodiment of the compounds of formula IH, M⁴ is —CH₂—, L⁵ is —C(R⁴)₂—, and Z is substituted or unsubstituted pyrrolyl.

In another embodiment of the compounds of formula IH, M⁴ is —CH(CH₃)—, L⁵ is —C(R⁴)₂—, and Z is substituted or unsubstituted pyrrolyl.

In another embodiment of the compounds of formula IH, M⁴ is —C(R⁴)₂—, L⁵ is —CH₂—, and Z is substituted or unsubstituted pyrrolyl.

In another embodiment of the compounds of formula IH, M⁴ is —CH₂—, L⁵ is —CH₂—, and Z is substituted or unsubstituted pyrrolyl.

In another embodiment of the compounds of formula IH, M⁴ is —CH(CH₃)—, L⁵ is —CH₂—, and Z is substituted or unsubstituted pyrrolyl.

In another embodiment of the compounds of formula IH, M⁴ is —C(R⁴)₂—, L⁵ is —CH(CH₃)—, and Z is substituted or unsubstituted pyrrolyl.

In another embodiment of the compounds of formula IH, M⁴ is —CH₂—, L⁵ is —CH(CH₃)—, and Z is substituted or unsubstituted pyrrolyl.

In another embodiment of the compounds of formula IH, M⁴ is —CH(CH₃)—, L⁵ is —CH(CH₃)—, and Z is substituted or unsubstituted pyrrolyl.

In another embodiment of the compounds of formula IH, M⁴ is —C(R⁴)₂—, L⁵ is —C(R⁴)₂—, and Z is alkoxyalkyl.

In another embodiment of the compounds of formula IH, M⁴ is —CH₂—, L⁵ is —C(R⁴)₂—, and Z is alkoxyalkyl.

In another embodiment of the compounds of formula IH, M⁴ is —CH(CH₃)—, L⁵ is —C(R⁴)₂—, and Z is alkoxyalkyl.

In another embodiment of the compounds of formula IH, M⁴ is —C(R⁴)₂—, L⁵ is —CH₂—, and Z is alkoxyalkyl.

In another embodiment of the compounds of formula IH, M⁴ is —CH₂—, L¹ is —CH₂—, and Z is alkoxyalkyl.

In another embodiment of the compounds of formula IH, M⁴ is —CH(CH₃)—, L⁵ is —CH₂—, and Z is alkoxyalkyl.

In another embodiment of the compounds of formula IH, M⁴ is —C(R⁴)₂—, L⁵ is —CH(CH₃)—, and Z is alkoxyalkyl.

In another embodiment of the compounds of formula IH, M⁴ is —CH₂—, L⁵ is —CH(CH₃)—, and Z is alkoxyalkyl.

In another embodiment of the compounds of formula IH, M⁴ is —CH(CH₃)—, L⁵ is —CH(CH₃)—, and Z is alkoxyalkyl.

In another embodiment, the compounds of the present invention, or pharmaceutically acceptable salts, solvates, prodrugs, tautomers, or esters thereof, have the structure of formula (IH-1):

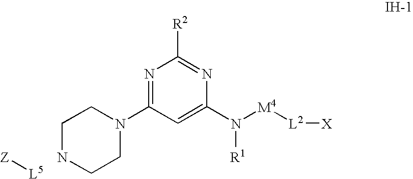

wherein R¹, R², M⁴, L², L⁵, n, X and Z are as defined herein.

In one embodiment of the compounds of formula IH-1, M⁴ is selected from the group consisting of —CH₂—, —CH(CH₃)—, and substituted or unsubstituted —CH(phenyl)-; L² is a covalent bond; L⁵ is —CH₂ or —CH(aminopropyl)-; X is selected from the group consisting of substituted or unsubstituted phenyl, and substituted or unsubstituted imidazolyl; Z is selected from the group consisting of substituted or unsubstituted phenyl, and substituted or unsubstituted imidazolyl; R¹ is H; and R² is H or —CF₃.

In one embodiment of the compounds of formula IH-1, M⁴ is —N(R⁵)—.

In one embodiment of the compounds of formula IH-1, M⁴ is —NH—.

In one embodiment of the compounds of formula IH-1, M⁴ is —N(alkyl)-.

In one embodiment of the compounds of formula IH-1, M⁴ is —N(R⁵)—, and L² is —C(R⁴)₂—.

In one embodiment of the compounds of formula IH-1, M⁴ is —N(R⁵)—, L² is —C(R⁴)₂—, and X is substituted or unsubstituted aryl or heteroaryl.

In one embodiment of the compounds of formula IH-1, M⁴ is —N(R⁵)—, L² is —C(R⁴)₂—, and X is substituted or unsubstituted phenyl.

In one embodiment of the compounds of formula IH-1, M⁴ is —N(R⁵)—, L² is —C(R⁴)₂—, and X is substituted or unsubstituted pyridyl.

In one embodiment of the compounds of formula IH-1, M⁴ is —N(R⁵)—, L² is —C(R⁴)₂—, and X is substituted or unsubstituted pyrimidyl.

In one embodiment of the compounds of formula IH-1, M⁴ is —N(R⁵)—, L² is —C(R⁴)₂—, and X is substituted or unsubstituted indolyl.

In one embodiment of the compounds of formula IH-1, M⁴ is —N(R⁵)—, L² is —C(R⁴)₂—, and X is substituted or unsubstituted quinolyl.

In one embodiment of the compounds of formula IH-1, M⁴ is —N(R⁵)—, L² is —C(R⁴)₂—, and X is substituted or unsubstituted benzimidazolyl.

In one embodiment of the compounds of formula IH-1, M⁴ is —N(R⁵)—, L² is —C(R⁴)₂—, and X is substituted or unsubstituted thienyl.

In one embodiment of the compounds of formula IH-1, M⁴ is —N(R⁵)—, L² is —C(R⁴)₂—, and X is substituted or unsubstituted furanyl.

In one embodiment of the compounds of formula IH-1, M⁴ is —N(R⁵)—, L² is —C(R⁴)₂—, and X is substituted or unsubstituted pyrrolyl.

In one embodiment of the compounds of formula IH-1, M⁴ is —C(R⁴)₂—.

In one embodiment of the compounds of formula IH-1, M⁴ is —CH₂—.

In one embodiment of the compounds of formula IH-1, M⁴ is —CH(CH₃)—.

In one embodiment of the compounds of formula IH-1, M⁴ is substituted or unsubstituted —CH(aryl)-.

In one embodiment of the compounds of formula IH-1, M⁴ is —C(R⁴)₂—, and L² is a covalent bond.

In one embodiment of the compounds of formula IH-1, M⁴ is —CH₂—, and L² is a covalent bond.

In one embodiment of the compounds of formula IH-1, M⁴ is —CH(CH₃)—, and L² is a covalent bond.

In one embodiment of the compounds of formula IH-1, M⁴ is substituted or unsubstituted —CH(aryl)-, and L² is a covalent bond.

In one embodiment of the compounds of formula IH-1, M⁴ is —C(R⁴)₂—, and L² is a —C(R⁴)₂—.

In one embodiment of the compounds of formula IH-1, M⁴ is —CH₂—, and L² is —C(R⁴)₂—.

In one embodiment of the compounds of formula IH-1, M⁴ is —CH(CH₃)—, and L² is —C(R⁴)₂—.

In one embodiment of the compounds of formula IH-1, M⁴ is substituted or unsubstituted —CH(aryl)-, and L² is —C(R⁴)₂—.

In one embodiment of the compounds of formula IH-1, M⁴ is —C(R⁴)₂—, and L² is a —C(O)—.

In one embodiment of the compounds of formula IH-1, M⁴ is —CH₂—, and L² is —C(O)—.

In one embodiment of the compounds of formula IH-1, M⁴ is —CH(CH₃)—, and L² is —C(O)—.

In one embodiment of the compounds of formula IH-1, M⁴ is substituted or unsubstituted —CH(aryl)-, and L² is —C(O)—.

In one embodiment of the compounds of formula IH-1, M⁴ is —C(R⁴)₂—, and L² is a —S(O)₂—.

In one embodiment of the compounds of formula IH-1, M⁴ is —CH₂—, and L² is —S(O)₂—.

In one embodiment of the compounds of formula IH-1, M⁴ is —CH(CH₃)—, and L² is —S(O)₂—.

In one embodiment of the compounds of formula IH-1, M⁴ is substituted or unsubstituted —CH(aryl)-, and L² is —S(O)₂—.

In one embodiment of the compounds of formula IH-1, M⁴ is —C(R⁴)₂—, L² is a covalent bond, and X is substituted or unsubstituted aryl.

In one embodiment of the compounds of formula IH-1, M⁴ is —CH₂—, L² is a covalent bond, and X is substituted or unsubstituted aryl.

In one embodiment of the compounds of formula IH-1, M⁴ is —CH(CH₃)—, L² is a covalent bond, and X is substituted or unsubstituted aryl.

In one embodiment of the compounds of formula IH-1, M⁴ is substituted or unsubstituted —CH(aryl)-, L² is a covalent bond, and X is substituted or unsubstituted aryl.

In one embodiment of the compounds of formula IH-1, M⁴ is —C(R⁴)₂—, L² is a —C(R⁴)₂—, and X is substituted or unsubstituted aryl.

In one embodiment of the compounds of formula IH-1, M⁴ is —CH₂—, L² is —C(R⁴)₂—, and X is substituted or unsubstituted aryl.

In one embodiment of the compounds of formula IH-1, M⁴ is —CH(CH₃)—, L² is —C(R⁴)₂—, and X is substituted or unsubstituted aryl or heteroaryl.

In one embodiment of the compounds of formula IH-1, M⁴ is substituted or unsubstituted —CH(aryl)-, L² is —C(R⁴)₂—, and X is substituted or unsubstituted aryl or heteroaryl.

In one embodiment of the compounds of formula IH-1, M is —C(R⁴)₂—, L² is a —C(O)—, and X is substituted or unsubstituted aryl or heteroaryl.

In one embodiment of the compounds of formula IH-1, M⁴ is —CH₂—, L² is —C(O)—, and X is substituted or unsubstituted aryl or heteroaryl.

In one embodiment of the compounds of formula IH-1, M⁴ is —CH(CH₃)—, L² is —C(O)—, and X is substituted or unsubstituted aryl or heteroaryl.

In one embodiment of the compounds of formula IH-1, M⁴ is substituted or unsubstituted —CH(aryl)-, L² is —C(O)—, and X is substituted or unsubstituted aryl or heteroaryl.

In one embodiment of the compounds of formula IH-1, M⁴ is —C(R⁴)₂—, L² is a —S(O)₂—, and X is substituted or unsubstituted aryl or heteroaryl.

In one embodiment of the compounds of formula IH-1, M is —CH₂—, L² is —S(O)₂—, and X is substituted or unsubstituted aryl or heteroaryl.

In one embodiment of the compounds of formula IH-1, M⁴ is —CH(CH₃)—, L² is —S(O)₂—, and X is substituted or unsubstituted aryl or heteroaryl.

In one embodiment of the compounds of formula IH-1, M⁴ is substituted or unsubstituted —CH(aryl)-, L² is —S(O)₂—, and X is substituted or unsubstituted aryl or heteroaryl.

In one embodiment of the compounds of formula IH-1, M⁴ is —C(R⁴)₂—, L² is a covalent bond, and X is substituted or unsubstituted aryl.

In one embodiment of the compounds of formula IH-1, M⁴ is —CH₂—, L² is a covalent bond, and X is substituted or unsubstituted aryl.

In one embodiment of the compounds of formula IH-1, M⁴ is —CH(CH₃)—, L² is a covalent bond, and X is substituted or unsubstituted aryl.

In one embodiment of the compounds of formula IH-1, M⁴ is substituted or unsubstituted —CH(aryl)-, L² is a covalent bond, and X is substituted or unsubstituted aryl.

In one embodiment of the compounds of formula IH-1, M⁴ is —C(R⁴)₂—, L² is a —C(R⁴)₂—, and X is substituted or unsubstituted aryl.

In one embodiment of the compounds of formula IH-1, M⁴ is —CH₂—, L² is —C(R⁴)₂—, and X is substituted or unsubstituted aryl.

In one embodiment of the compounds of formula IH-1, M⁴ is —CH(CH₃)—, L² is —C(R⁴)₂—, and X is substituted or unsubstituted phenyl.

In one embodiment of the compounds of formula IH-1, M⁴ is substituted or unsubstituted —CH(aryl)-, L² is —C(R⁴)₂—, and X is substituted or unsubstituted phenyl.

In one embodiment of the compounds of formula IH-1, M⁴ is —C(R⁴)₂—, L² is a —C(O)—, and X is substituted or unsubstituted phenyl.

In one embodiment of the compounds of formula IH-1, M⁴ is —CH₂—, L² is —C(O)—, and X is substituted or unsubstituted phenyl.

In one embodiment of the compounds of formula IH-1, M⁴ is —CH(CH₃)—, L² is —C(O)—, and X is substituted or unsubstituted phenyl.

In one embodiment of the compounds of formula IH-1, M⁴ is substituted or unsubstituted —CH(aryl)-, L² is —C(O)—, and X is substituted or unsubstituted phenyl.

In one embodiment of the compounds of formula IH-1, M⁴ is —C(R⁴)₂—, L² is a —S(O)₂—, and X is substituted or unsubstituted phenyl.

In one embodiment of the compounds of formula IH-1, M⁴ is —CH₂—, L² is —S(O)₂—, and X is substituted or unsubstituted phenyl.

In one embodiment of the compounds of formula IH-1, M⁴ is —CH(CH₃)—, L² is —S(O)₂—, and X is substituted or unsubstituted phenyl.

In one embodiment of the compounds of formula IH-1, M⁴ is substituted or unsubstituted —CH(aryl)-, L² is —S(O)₂—, and X is substituted or unsubstituted phenyl.

In one embodiment of the compounds of formula IH-1, M⁴ is —C(R⁴)₂—, L² is a covalent bond, and X is substituted or unsubstituted aryl.

In one embodiment of the compounds of formula IH-1, M⁴ is —CH₂—, L² is a covalent bond, and X is substituted or unsubstituted aryl.

In one embodiment of the compounds of formula IH-1, M⁴ is —CH(CH₃)—, L² is a covalent bond, and X is substituted or unsubstituted aryl.

In one embodiment of the compounds of formula IH-1, M⁴ is substituted or unsubstituted —CH(aryl)-, L² is a covalent bond, and X is substituted or unsubstituted aryl.

In one embodiment of the compounds of formula IH-1, M⁴ is —C(R⁴)₂—, L² is a —C(R⁴)₂—, and X is substituted or unsubstituted aryl.

In one embodiment of the compounds of formula IH-1, M⁴ is —CH₂—, L² is —C(R⁴)₂—, and X is substituted or unsubstituted aryl.

In one embodiment of the compounds of formula IH-1, M⁴ is —CH(CH₃)—, L² is —C(R⁴)₂—, and X is substituted or unsubstituted pyridyl.

In one embodiment of the compounds of formula IH-1, M⁴ is substituted or unsubstituted —CH(aryl)-, L² is —C(R⁴)₂—, and X is substituted or unsubstituted pyridyl.

In one embodiment of the compounds of formula IH-1, M⁴ is —C(R⁴)₂—, L² is a —C(O)—, and X is substituted or unsubstituted pyridyl.

In one embodiment of the compounds of formula IH-1, M⁴ is —CH₂—, L² is —C(O)—, and X is substituted or unsubstituted pyridyl.

In one embodiment of the compounds of formula IH-1, M⁴ is —CH(CH₃)—, L² is —C(O)—, and X is substituted or unsubstituted pyridyl.

In one embodiment of the compounds of formula IH-1, M⁴ is substituted or unsubstituted —CH(aryl)-, L² is —C(O)—, and X is substituted or unsubstituted pyridyl.

In one embodiment of the compounds of formula IH-1, M⁴ is —C(R⁴)₂—, L² is a —S(O)₂—, and X is substituted or unsubstituted pyridyl.

In one embodiment of the compounds of formula IH-1, M⁴ is —CH₂—, L² is —S(O)₂—, and X is substituted or unsubstituted pyridyl.

In one embodiment of the compounds of formula IH-1, M⁴ is —CH(CH₃)—, L² is —S(O)₂—, and X is substituted or unsubstituted pyridyl.

In one embodiment of the compounds of formula IH-1, M⁴ is substituted or unsubstituted —CH(aryl)-, L² is —S(O)₂—, and X is substituted or unsubstituted pyridyl.

In one embodiment of the compounds of formula IH-1, M⁴ is —C(R⁴)₂—, L² is a covalent bond, and X is substituted or unsubstituted aryl.

In one embodiment of the compounds of formula IH-1, M⁴ is —CH₂—, L² is a covalent bond, and X is substituted or unsubstituted aryl.

In one embodiment of the compounds of formula IH-1, M⁴ is —CH(CH₃)—, L² is a covalent bond, and X is substituted or unsubstituted aryl.

In one embodiment of the compounds of formula IH-1, M⁴ is substituted or unsubstituted —CH(aryl)-, L² is a covalent bond, and X is substituted or unsubstituted aryl.

In one embodiment of the compounds of formula IH-1, M⁴ is —C(R⁴)₂—, L² is a —C(R⁴)₂—, and X is substituted or unsubstituted aryl.

In one embodiment of the compounds of formula IH-1, M⁴ is —CH₂—, L² is —C(R⁴)₂—, and X is substituted or unsubstituted aryl.

In one embodiment of the compounds of formula IH-1, M⁴ is —CH(CH₃)—, L² is —C(R⁴)₂—, and X is substituted or unsubstituted pyrimidyl.

In one embodiment of the compounds of formula IH-1, M⁴ is substituted or unsubstituted —CH(aryl)-, L² is —C(R⁴)₂—, and X is substituted or unsubstituted pyrimidyl.

In one embodiment of the compounds of formula IH-1, M⁴ is —C(R⁴)₂—, L² is a —C(O)—, and X is substituted or unsubstituted pyrimidyl.

In one embodiment of the compounds of formula IH-1, M⁴ is —CH₂—, L² is —C(O)—, and X is substituted or unsubstituted pyrimidyl.

In one embodiment of the compounds of formula IH-1, M⁴ is —CH(CH₃)—, L² is —C(O)—, and X is substituted or unsubstituted pyrimidyl.

In one embodiment of the compounds of formula IH-1, M⁴ is substituted or unsubstituted —CH(aryl)-, L² is —C(O)—, and X is substituted or unsubstituted pyrimidyl.

In one embodiment of the compounds of formula IH-1, M⁴ is —C(R⁴)₂—, L² is a —S(O)₂—, and X is substituted or unsubstituted pyrimidyl.

In one embodiment of the compounds of formula IH-1, M⁴ is —CH₂—, L² is —S(O)₂—, and X is substituted or unsubstituted pyrimidyl.

In one embodiment of the compounds of formula IH-1, M⁴ is —CH(CH₃)—, L² is —S(O)₂—, and X is substituted or unsubstituted pyrimidyl.

In one embodiment of the compounds of formula IH-1, M⁴ is substituted or unsubstituted —CH(aryl)-, L² is —S(O)₂—, and X is substituted or unsubstituted pyrimidyl.

In one embodiment of the compounds of formula IH-1, M⁴ is —C(R⁴)₂—, L² is a covalent bond, and X is substituted or unsubstituted aryl.

In one embodiment of the compounds of formula IH-1, M⁴ is —CH₂—, L² is a covalent bond, and X is substituted or unsubstituted aryl.

In one embodiment of the compounds of formula IH-1, M⁴ is —CH(CH₃)—, L² is a covalent bond, and X is substituted or unsubstituted aryl.

In one embodiment of the compounds of formula IH-1, M⁴ is substituted or unsubstituted —CH(aryl)-, L² is a covalent bond, and X is substituted or unsubstituted aryl.

In one embodiment of the compounds of formula IH-1, M⁴ is —C(R⁴)₂—, L² is a —C(R⁴)₂—, and X is substituted or unsubstituted aryl.

In one embodiment of the compounds of formula IH-1, M⁴ is —CH₂—, L² is —C(R⁴)₂—, and X is substituted or unsubstituted aryl.

In one embodiment of the compounds of formula IH-1, M⁴ is —CH(CH₃)—, L² is —C(R⁴)₂—, and X is substituted or unsubstituted quinolyl.

In one embodiment of the compounds of formula IH-1, M⁴ is substituted or unsubstituted —CH(aryl)-, L² is —C(R⁴)₂—, and X is substituted or unsubstituted quinolyl.

In one embodiment of the compounds of formula IH-1, M⁴ is —C(R⁴)₂—, L² is a —C(O)—, and X is substituted or unsubstituted quinolyl.

In one embodiment of the compounds of formula IH-1, M⁴ is —CH₂—, L² is —C(O)—, and X is substituted or unsubstituted quinolyl.

In one embodiment of the compounds of formula IH-1, M⁴ is —CH(CH₃)—, L² is —C(O)—, and X is substituted or unsubstituted quinolyl.

In one embodiment of the compounds of formula IH-1, M⁴ is substituted or unsubstituted —CH(aryl)-, L² is —C(O)—, and X is substituted or unsubstituted quinolyl.

In one embodiment of the compounds of formula IH-1, M⁴ is —C(R⁴)₂—, L² is a —S(O)₂—, and X is substituted or unsubstituted quinolyl.

In one embodiment of the compounds of formula IH-1, M⁴ is —CH₂—, L² is —S(O)₂—, and X is substituted or unsubstituted quinolyl.

In one embodiment of the compounds of formula IH-1, M⁴ is —CH(CH₃)—, L² is —S(O)₂—, and X is substituted or unsubstituted quinolyl.

In one embodiment of the compounds of formula IH-1, M⁴ is substituted or unsubstituted —CH(aryl)-, L² is —S(O)₂—, and X is substituted or unsubstituted quinolyl.

In one embodiment of the compounds of formula IH-1, M⁴ is —C(R⁴)₂—, L² is a covalent bond, and X is substituted or unsubstituted aryl.

In one embodiment of the compounds of formula IH-1, M⁴ is —CH₂—, L² is a covalent bond, and X is substituted or unsubstituted aryl.

In one embodiment of the compounds of formula IH-1, M⁴ is —CH(CH₃)—, L² is a covalent bond, and X is substituted or unsubstituted aryl.

In one embodiment of the compounds of formula IH-1, M⁴ is substituted or unsubstituted —CH(aryl)-, L² is a covalent bond, and X is substituted or unsubstituted aryl.

In one embodiment of the compounds of formula IH-1, M⁴ is —C(R⁴)₂—, L² is a —C(R⁴)₂—, and X is substituted or unsubstituted aryl.

In one embodiment of the compounds of formula IH-1, M⁴ is —CH₂—, L² is —C(R⁴)₂—, and X is substituted or unsubstituted aryl.

In one embodiment of the compounds of formula IH-1, M⁴ is —CH(CH₃)—, L² is —C(R⁴)₂—, and X is substituted or unsubstituted indolyl.

In one embodiment of the compounds of formula IH-1, M⁴ is substituted or unsubstituted —CH(aryl)-, L² is —C(R⁴)₂—, and X is substituted or unsubstituted indolyl.

In one embodiment of the compounds of formula IH-1, M⁴ is —C(R⁴)₂—, L² is a —C(O)—, and X is substituted or unsubstituted indolyl.

In one embodiment of the compounds of formula IH-1, M⁴ is —CH₂—, L² is —C(O)—, and X is substituted or unsubstituted indolyl.

In one embodiment of the compounds of formula IH-1, M⁴ is —CH(CH₃)—, L² is —C(O)—, and X is substituted or unsubstituted indolyl.

In one embodiment of the compounds of formula IH-1, M⁴ is substituted or unsubstituted —CH(aryl)-, L² is —C(O)—, and X is substituted or unsubstituted indolyl.

In one embodiment of the compounds of formula IH-1, M⁴ is —C(R⁴)₂—, L² is a —S(O)₂—, and X is substituted or unsubstituted indolyl.

In one embodiment of the compounds of formula IH-1, M⁴ is —CH₂—, L² is —S(O)₂—, and X is substituted or unsubstituted indolyl.

In one embodiment of the compounds of formula IH-1, M⁴ is —CH(CH₃)—, L² is —S(O)₂—, and X is substituted or unsubstituted indolyl.

In one embodiment of the compounds of formula IH-1, M⁴ is substituted or unsubstituted —CH(aryl)-, L² is —S(O)₂—, and X is substituted or unsubstituted indolyl.

In one embodiment of the compounds of formula IH-1, M⁴ is —C(R⁴)₂—, L² is a covalent bond, and X is substituted or unsubstituted aryl.

In one embodiment of the compounds of formula IH-1, M⁴ is —CH₂—, L² is a covalent bond, and X is substituted or unsubstituted aryl.

In one embodiment of the compounds of formula IH-1, M⁴ is —CH(CH₃)—, L² is a covalent bond, and X is substituted or unsubstituted aryl.

In one embodiment of the compounds of formula IH-1, M⁴ is substituted or unsubstituted —CH(aryl)-, L² is a covalent bond, and X is substituted or unsubstituted aryl.

In one embodiment of the compounds of formula IH-1, M⁴ is —C(R⁴)₂—, L² is a —C(R⁴)₂—, and X is substituted or unsubstituted aryl.

In one embodiment of the compounds of formula IH-1, M is —CH₂—, L² is —C(R⁴)₂—, and X is substituted or unsubstituted aryl.

In one embodiment of the compounds of formula IH-1, M⁴ is —CH(CH₃)—, L² is —C(R⁴)₂—, and X is substituted or unsubstituted benzimidazolyl.

In one embodiment of the compounds of formula IH-1, M⁴ is substituted or unsubstituted —CH(aryl)-, L² is —C(R⁴)₂—, and X is substituted or unsubstituted benzimidazolyl.

In one embodiment of the compounds of formula IH-1, M⁴ is —C(R⁴)₂—, L² is a —C(O)—, and X is substituted or unsubstituted benzimidazolyl.

In one embodiment of the compounds of formula IH-1, M⁴ is —CH₂—, L² is —C(O)—, and X is substituted or unsubstituted benzimidazolyl.

In one embodiment of the compounds of formula IH-1, M⁴ is —CH(CH₃)—, L² is —C(O)—, and X is substituted or unsubstituted benzimidazolyl.

In one embodiment of the compounds of formula IH-1, M⁴ is substituted or unsubstituted —CH(aryl)-, L² is —C(O)—, and X is substituted or unsubstituted benzimidazolyl.

In one embodiment of the compounds of formula IH-1, M⁴ is —C(R⁴)₂—, L² is a —S(O)₂—, and X is substituted or unsubstituted benzimidazolyl.

In one embodiment of the compounds of formula IH-1, M⁴ is —CH₂—, L² is —S(O)₂—, and X is substituted or unsubstituted benzimidazolyl.

In one embodiment of the compounds of formula IH-1, M⁴ is —CH(CH₃)—, L² is —S(O)₂—, and X is substituted or unsubstituted benzimidazolyl.

In one embodiment of the compounds of formula IH-1, M⁴ is substituted or unsubstituted —CH(aryl)-, L² is —S(O)₂—, and X is substituted or unsubstituted benzimidazolyl.

In one embodiment of the compounds of formula IH-1, M⁴ is —C(R⁴)₂—, L² is a covalent bond, and X is substituted or unsubstituted aryl.

In one embodiment of the compounds of formula IH-1, M⁴ is —CH₂—, L² is a covalent bond, and X is substituted or unsubstituted aryl.

In one embodiment of the compounds of formula IH-1, M⁴ is —CH(CH₃)—, L² is a covalent bond, and X is substituted or unsubstituted aryl.

In one embodiment of the compounds of formula IH-1, M⁴ is substituted or unsubstituted —CH(aryl)-, L² is a covalent bond, and X is substituted or unsubstituted aryl.

In one embodiment of the compounds of formula IH-1, M⁴ is —C(R⁴)₂—, L² is a —C(R⁴)₂—, and X is substituted or unsubstituted aryl.

In one embodiment of the compounds of formula IH-1, M⁴ is —CH₂—, L² is —C(R⁴)₂—, and X is substituted or unsubstituted aryl.

In one embodiment of the compounds of formula IH-1, M⁴ is —CH(CH₃)—, L² is —C(R⁴)₂—, and X is substituted or unsubstituted thienyl.

In one embodiment of the compounds of formula IH-1, M⁴ is substituted or unsubstituted —CH(aryl)-, L² is —C(R⁴)₂—, and X is substituted or unsubstituted thienyl.

In one embodiment of the compounds of formula IH-1, M⁴ is —C(R⁴)₂—, L² is —C(O)—, and X is substituted or unsubstituted thienyl.

In one embodiment of the compounds of formula IH-1, M⁴ is —CH₂—, L² is —C(O)—, and X is substituted or unsubstituted thienyl.

In one embodiment of the compounds of formula IH-1, M⁴ is —CH(CH₃)—, L² is —C(O)—, and X is substituted or unsubstituted thienyl.

In one embodiment of the compounds of formula IH-1, M⁴ is substituted or unsubstituted —CH(aryl)-, L² is —C(O)—, and X is substituted or unsubstituted thienyl.

In one embodiment of the compounds of formula IH-1, M⁴ is —C(R⁴)₂—, L² is —S(O)₂—, and X is substituted or unsubstituted thienyl.

In one embodiment of the compounds of formula IH-1, M⁴ is —CH₂—, L² is —S(O)₂—, and X is substituted or unsubstituted thienyl.

In one embodiment of the compounds of formula IH-1, M⁴ is —CH(CH₃)—, L² is —S(O)₂—, and X is substituted or unsubstituted thienyl.

In one embodiment of the compounds of formula IH-1, M⁴ is substituted or unsubstituted —CH(aryl)-, L² is —S(O)₂—, and X is substituted or unsubstituted thienyl.

In one embodiment of the compounds of formula IH-1, M⁴ is —C(R⁴)₂—, L² is a covalent bond, and X is substituted or unsubstituted aryl.

In one embodiment of the compounds of formula IH-1, M⁴ is —CH₂—, L² is a covalent bond, and X is substituted or unsubstituted aryl.

In one embodiment of the compounds of formula IH-1, M⁴ is —CH(CH₃)—, L² is a covalent bond, and X is substituted or unsubstituted aryl.

In one embodiment of the compounds of formula IH-1, M⁴ is substituted or unsubstituted —CH(aryl)-, L² is a covalent bond, and X is substituted or unsubstituted aryl.

In one embodiment of the compounds of formula IH-1, M⁴ is —C(R⁴)₂—, L² is a —C(R⁴)₂—, and X is substituted or unsubstituted aryl.

In one embodiment of the compounds of formula IH-1, M⁴ is —CH₂—, L² is —C(R⁴)₂—, and X is substituted or unsubstituted aryl.

In one embodiment of the compounds of formula IH-1, M⁴ is —CH(CH₃)—, L² is —C(R⁴)₂—, and X is substituted or unsubstituted furanyl.

In one embodiment of the compounds of formula IH-1, M⁴ is substituted or unsubstituted —CH(aryl)-, L² is —C(R⁴)₂—, and X is substituted or unsubstituted furanyl.

In one embodiment of the compounds of formula IH-1, M⁴ is —C(R⁴)₂—, L² is —C(O)—, and X is substituted or unsubstituted furanyl.

In one embodiment of the compounds of formula IH-1, M⁴ is —CH₂—, L² is —C(O)—, and X is substituted or unsubstituted furanyl.

In one embodiment of the compounds of formula IH-1, M⁴ is —CH(CH₃)—, L² is —C(O)—, and X is substituted or unsubstituted furanyl.

In one embodiment of the compounds of formula IH-1, M⁴ is substituted or unsubstituted —CH(aryl)-, L² is —C(O)—, and X is substituted or unsubstituted furanyl.

In one embodiment of the compounds of formula IH-1, M⁴ is —C(R⁴)₂—, L² is —S(O)₂—, and X is substituted or unsubstituted furanyl.

In one embodiment of the compounds of formula IH-1, M⁴ is —CH₂—, L² is —S(O)₂—, and X is substituted or unsubstituted furanyl.

In one embodiment of the compounds of formula IH-1, M⁴ is —CH(CH₃)—, L² is —S(O)₂—, and X is substituted or unsubstituted furanyl.

In one embodiment of the compounds of formula IH-1, M⁴ is substituted or unsubstituted —CH(aryl)-, L² is —S(O)₂—, and X is substituted or unsubstituted furanyl.

In one embodiment of the compounds of formula IH-1, M⁴ is —C(R⁴)₂—, L² is a covalent bond, and X is substituted or unsubstituted aryl.

In one embodiment of the compounds of formula IH-1, M⁴ is —CH₂—, L² is a covalent bond, and X is substituted or unsubstituted aryl.

In one embodiment of the compounds of formula IH-1, M⁴ is —CH(CH₃)—, L² is a covalent bond, and X is substituted or unsubstituted aryl.

In one embodiment of the compounds of formula IH-1, M⁴ is substituted or unsubstituted —CH(aryl)-, L² is a covalent bond, and X is substituted or unsubstituted aryl.

In one embodiment of the compounds of formula IH-1, M⁴ is —C(R⁴)₂—, L² is a —C(R⁴)₂—, and X is substituted or unsubstituted aryl.

In one embodiment of the compounds of formula IH-1, M⁴ is —CH₂—, L² is —C(R⁴)₂—, and X is substituted or unsubstituted aryl.

In one embodiment of the compounds of formula IH-1, M⁴ is —CH(CH₃)—, L² is —C(R⁴)₂—, and X is substituted or unsubstituted pyrrolyl.

In one embodiment of the compounds of formula IH-1, M⁴ is substituted or unsubstituted —CH(aryl)-, L² is —C(R⁴)₂—, and X is substituted or unsubstituted pyrrolyl.

In one embodiment of the compounds of formula IH-1, M⁴ is —C(R⁴)₂—, L² is —C(O)—, and X is substituted or unsubstituted pyrrolyl.

In one embodiment of the compounds of formula IH-1, M⁴ is —CH₂—, L² is —C(O)—, and X is substituted or unsubstituted pyrrolyl.

In one embodiment of the compounds of formula IH-1, M⁴ is —CH(CH₃)—, L² is —C(O)—, and X is substituted or unsubstituted pyrrolyl.

In one embodiment of the compounds of formula IH-1, M⁴ is substituted or unsubstituted —CH(aryl)-, L² is —C(O)—, and X is substituted or unsubstituted pyrrolyl.

In one embodiment of the compounds of formula IH-1, M⁴ is —C(R⁴)₂—, L² is —S(O)₂—, and X is substituted or unsubstituted pyrrolyl.

In one embodiment of the compounds of formula IH-1, M⁴ is —CH₂—, L² is —S(O)₂—, and X is substituted or unsubstituted pyrrolyl.

In one embodiment of the compounds of formula IH-1, M⁴ is —CH(CH₃)—, L² is —S(O)₂—, and X is substituted or unsubstituted pyrrolyl.

In one embodiment of the compounds of formula IH-1, M⁴ is substituted or unsubstituted —CH(aryl)-, L² is —S(O)₂—, and X is substituted or unsubstituted pyrrolyl.

In one embodiment of the compounds of formula IH-1, L⁵ is —C(R⁴)₂—.

In one embodiment of the compounds of formula IH-1, L⁵ is —CH₂—.

In one embodiment of the compounds of formula IH-1, L⁵ is —CH(CH₃)—.

In one embodiment of the compounds of formula IH-1, L⁵ is —CH(aminoalkyl)-.

In one embodiment of the compounds of formula IH-1, L⁵ is —CH(H₂Nalkyl)-.

In one embodiment of the compounds of formula IH-1, L⁵ is —CH((alkyl)HNalkyl)-.

In one embodiment of the compounds of formula IH-1, L⁵ is —CH((alkyl)₂Nalkyl)-.

In one embodiment of the compounds of formula IH-1, L⁵ is —C(R⁴)₂—, and Z is substituted or unsubstituted aryl or heteroaryl.

In one embodiment of the compounds of formula IH-1, L⁵ is —CH₂—, and Z is substituted or unsubstituted aryl or heteroaryl.

In one embodiment of the compounds of formula IH-1, L⁵ is —CH(CH₃)—, and Z is substituted or unsubstituted aryl or heteroaryl.

In one embodiment of the compounds of formula IH-1, L⁵ is —CH(aminoalkyl)-, and Z is substituted or unsubstituted aryl or heteroaryl.

In one embodiment of the compounds of formula IH-1, L⁵ is —CH(H₂Nalkyl)-, and Z is substituted or unsubstituted aryl or heteroaryl.

In one embodiment of the compounds of formula IH-1, L⁵ is —CH((alkyl)HNalkyl)-, and Z is substituted or unsubstituted aryl or heteroaryl.

In one embodiment of the compounds of formula IH-1, L⁵ is —CH((alkyl)₂Nalkyl)-, and Z is substituted or unsubstituted aryl or heteroaryl.

In one embodiment of the compounds of formula IH-1, L⁵ is —C(R⁴)₂—, and Z is substituted or unsubstituted phenyl.

In one embodiment of the compounds of formula IH-1, L⁵ is —CH₂—, and Z is substituted or unsubstituted phenyl.

In one embodiment of the compounds of formula IH-1, L⁵ is —CH(CH₃)—, and Z is substituted or unsubstituted phenyl.

In one embodiment of the compounds of formula IH-1, L⁵ is —CH(aminoalkyl)-, and Z is substituted or unsubstituted phenyl.

In one embodiment of the compounds of formula IH-1, L⁵ is —CH(H₂Nalkyl)-, and Z is substituted or unsubstituted phenyl.

In one embodiment of the compounds of formula IH-1, L⁵ is —CH((alkyl)HNalkyl)-, and Z is substituted or unsubstituted phenyl.

In one embodiment of the compounds of formula IH-1, L⁵ is —CH((alkyl)₂Nalkyl)-, and Z is substituted or unsubstituted phenyl.

In one embodiment of the compounds of formula IH-1, L⁵ is —C(R⁴)₂—, and Z is substituted or unsubstituted pyridyl.

In one embodiment of the compounds of formula IH-1, L⁵ is —CH₂—, and Z is substituted or unsubstituted pyridyl.

In one embodiment of the compounds of formula IH-1, L⁵ is —CH(CH₃)—, and Z is substituted or unsubstituted pyridyl.

In one embodiment of the compounds of formula IH-1, L⁵ is —CH(aminoalkyl)-, and Z is substituted or unsubstituted pyridyl.

In one embodiment of the compounds of formula IH-1, L⁵ is —CH(H₂Nalkyl)-, and Z is substituted or unsubstituted pyridyl.

In one embodiment of the compounds of formula IH-1, L⁵ is —CH((alkyl)HNalkyl)-, and Z is substituted or unsubstituted pyridyl.

In one embodiment of the compounds of formula IH-1, L⁵ is —CH((alkyl)₂Nalkyl)-, and Z is substituted or unsubstituted pyridyl.

In one embodiment of the compounds of formula IH-1, L⁵ is —C(R⁴)₂—, and Z is substituted or unsubstituted pyrimidyl.

In one embodiment of the compounds of formula IH-1, L⁵ is —CH₂—, and Z is substituted or unsubstituted pyrimidyl.

In one embodiment of the compounds of formula IH-1, L⁵ is —CH(CH₃)—, and Z is substituted or unsubstituted pyrimidyl.

In one embodiment of the compounds of formula IH-1, L⁵ is —CH(aminoalkyl)-, and Z is substituted or unsubstituted pyrimidyl.

In one embodiment of the compounds of formula IH-1, L⁵ is —CH(H₂Nalkyl)-, and Z is substituted or unsubstituted pyrimidyl.

In one embodiment of the compounds of formula IH-1, L⁵ is —CH((alkyl)HNalkyl)-, and Z is substituted or unsubstituted pyrimidyl.

In one embodiment of the compounds of formula IH-1, L⁵ is —CH((alkyl)₂Nalkyl)-, and Z is substituted or unsubstituted pyrimidyl.

In one embodiment of the compounds of formula IH-1, L⁵ is —C(R⁴)₂—, and Z is substituted or unsubstituted purinyl.

In one embodiment of the compounds of formula IH-1, L⁵ is —CH₂—, and Z is substituted or unsubstituted purinyl.

In one embodiment of the compounds of formula IH-1, L⁵ is —CH(CH₃)—, and Z is substituted or unsubstituted purinyl.

In one embodiment of the compounds of formula IH-1, L⁵ is —CH(aminoalkyl)-, and Z is substituted or unsubstituted purinyl.

In one embodiment of the compounds of formula IH-1, L⁵ is —CH(H₂Nalkyl)-, and Z is substituted or unsubstituted purinyl.

In one embodiment of the compounds of formula IH-1, L⁵ is —CH((alkyl)HNalkyl)-, and Z is substituted or unsubstituted purinyl.

In one embodiment of the compounds of formula IH-1, L⁵ is —CH((alkyl)₂Nalkyl)-, and Z is substituted or unsubstituted purinyl.

In one embodiment of the compounds of formula IH-1, L⁵ is —C(R⁴)₂—, and Z is substituted or unsubstituted quinolyl.

In one embodiment of the compounds of formula IH-1, L⁵ is —CH₂—, and Z is substituted or unsubstituted quinolyl.

In one embodiment of the compounds of formula IH-1, L⁵ is —CH(CH₃)—, and Z is substituted or unsubstituted quinolyl.

In one embodiment of the compounds of formula IH-1, L⁵ is —CH(aminoalkyl)-, and Z is substituted or unsubstituted quinolyl.

In one embodiment of the compounds of formula IH-1, L⁵ is —CH(H₂Nalkyl)-, and Z is substituted or unsubstituted quinolyl.

In one embodiment of the compounds of formula IH-1, L⁵ is —CH((alkyl)HNalkyl)-, and Z is substituted or unsubstituted quinolyl.

In one embodiment of the compounds of formula IH-1, L⁵ is —CH((alkyl)₂Nalkyl)-, and Z is substituted or unsubstituted quinolyl.

In one embodiment of the compounds of formula IH-1, L⁵ is —C(R⁴)₂—, and Z is substituted or unsubstituted indolyl.

In one embodiment of the compounds of formula IH-1, L⁵ is —CH₂—, and Z is substituted or unsubstituted indolyl.

In one embodiment of the compounds of formula IH-1, L⁵ is —CH(CH₃)—, and Z is substituted or unsubstituted indolyl.

In one embodiment of the compounds of formula IH-1, L⁵ is —CH(aminoalkyl)-, and Z is substituted or unsubstituted indolyl.

In one embodiment of the compounds of formula IH-1, L⁵ is —CH(H₂Nalkyl)-, and Z is substituted or unsubstituted indolyl.

In one embodiment of the compounds of formula IH-1, L⁵ is —CH((alkyl)HNalkyl)-, and Z is substituted or unsubstituted indolyl.

In one embodiment of the compounds of formula IH-1, L⁵ is —CH((alkyl)₂Nalkyl)-, and Z is substituted or unsubstituted indolyl.

In one embodiment of the compounds of formula IH-1, L⁵ is —C(R⁴)₂—, and Z is substituted or unsubstituted thienyl.

In one embodiment of the compounds of formula IH-1, L⁵ is —CH₂—, and Z is substituted or unsubstituted thienyl.

In one embodiment of the compounds of formula IH-1, L⁵ is —CH(CH₃)—, and Z is substituted or unsubstituted thienyl.

In one embodiment of the compounds of formula IH-1, L⁵ is —CH(aminoalkyl)-, and Z is substituted or unsubstituted thienyl.

In one embodiment of the compounds of formula IH-1, L⁵ is —CH(H₂Nalkyl)-, and Z is substituted or unsubstituted thienyl.

In one embodiment of the compounds of formula IH-1, L⁵ is —CH((alkyl)HNalkyl)-, and Z is substituted or unsubstituted thienyl.

In one embodiment of the compounds of formula IH-1, L⁵ is —CH((alkyl)₂Nalkyl)-, and Z is substituted or unsubstituted thienyl.

In one embodiment of the compounds of formula IH-1, L⁵ is —C(R⁴)₂—, and Z is substituted or unsubstituted furanyl.

In one embodiment of the compounds of formula IH-1, L⁵ is —CH₂—, and Z is substituted or unsubstituted furanyl.

In one embodiment of the compounds of formula IH-1, L⁵ is —CH(CH₃)—, and Z is substituted or unsubstituted furanyl.

In one embodiment of the compounds of formula IH-1, L⁵ is —CH(aminoalkyl)-, and Z is substituted or unsubstituted furanyl.

In one embodiment of the compounds of formula IH-1, L⁵ is —CH(H₂Nalkyl)-, and Z is substituted or unsubstituted furanyl.

In one embodiment of the compounds of formula IH-1, L⁵ is —CH((alkyl)HNalkyl)-, and Z is substituted or unsubstituted furanyl.

In one embodiment of the compounds of formula IH-1, L⁵ is —CH((alkyl)₂Nalkyl)-, and Z is substituted or unsubstituted furanyl.

In one embodiment of the compounds of formula IH-1, L⁵ is —C(R⁴)₂—, and Z is substituted or unsubstituted pyrrolyl.

In one embodiment of the compounds of formula IH-1, L⁵ is —CH₂—, and Z is substituted or unsubstituted pyrrolyl.

In one embodiment of the compounds of formula IH-1, L⁵ is —CH(CH₃)—, and Z is substituted or unsubstituted pyrrolyl.

In one embodiment of the compounds of formula IH-1, L⁵ is —CH(aminoalkyl)-, and Z is substituted or unsubstituted pyrrolyl.

In one embodiment of the compounds of formula IH-1, L⁵ is —CH(H₂Nalkyl)-, and Z is substituted or unsubstituted pyrrolyl.

In one embodiment of the compounds of formula IH-1, L⁵ is —CH((alkyl)HNalkyl)-, and Z is substituted or unsubstituted pyrrolyl.

In one embodiment of the compounds of formula IH-1, L⁵ is —CH((alkyl)₂Nalkyl)-, and Z is substituted or unsubstituted pyrrolyl.

In another embodiment, the compounds of the present invention, or pharmaceutically acceptable salts, solvates, prodrugs, tautomers, or esters thereof, have the structure of formula (II):

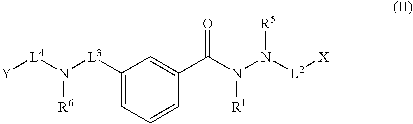

wherein L², L³, L⁴, R¹, R⁵, R⁶, X and Y are as defined herein.

In another embodiment of the compounds of formula (II),
L² is selected from the group consisting of a covalent bond, —S(O)₂—, —S(O)—, —C(O)—, —C(O)O—, —C(R⁴)₂—, and —C(R⁴)═;
L³ is selected from the group consisting of —C(O)—, —C(R⁴)₂—, —S(O)₂—, and —S(O)—;
L⁴ is selected from the group consisting of a covalent bond, —C(R⁴)₂—, —C(O)—, —S(O)₂—, and —S(O)—;
X is selected from the group consisting of H, alkyl, substituted or unsubstituted aryl, substituted or unsubstituted arylalkyl, substituted or unsubstituted heteroaryl, substituted or unsubstituted heterocyclyl, substituted or unsubstituted cycloalkyl, substituted or unsubstituted amino, and substituted or unsubstituted amido;
Y is selected from the group consisting of substituted or unsubstituted heteroaryl, substituted or unsubstituted aryl, hydroxyalkyl, alkoxyalkyl, aminoalkyl, substituted or unsubstituted cycloalkyl, substituted or unsubstituted arylalkyl, substituted or unsubstituted —C(O)-heterocyclyl, substituted or unsubstituted amino, substituted or unsubstituted arylalkylamino, substituted or unsubstituted heteroarylalkylamino, substituted or unsubstituted amidoalkyl, substituted or unsubstituted amido, substituted or unsubstituted heterocyclyl, and alkyl;
R¹ is selected from the group consisting of H, alkyl, alkenyl, aminoalkyl, substituted or unsubstituted arylalkyl, substituted or unsubstituted —S(O)₂-aryl, substituted or unsubstituted —S(O)₂-heteroaryl, substituted or unsubstituted heteroarylalkyl, substituted or unsubstituted cycloalkyl, substituted or unsubstituted aryl, substituted or unsubstituted heteroaryl, acyl, carboxy, alkoxycarbonyl, and aminocarbonyl;
R⁵ is selected from the group consisting of H, substituted or unsubstituted heteroarylalkyl, alkyl, substituted or unsubstituted aminoalkyl, substituted or unsubstituted cycloalkyl, substituted or unsubstituted arylalkyl, substituted or unsubstituted aryl, substituted or unsubstituted heteroaryl, acyl, carboxy, alkoxycarbonyl, and substituted or unsubstituted aminocarbonyl; or
R⁵ and R¹, together with the nitrogen atoms to which they are each attached, form a substituted or unsubstituted heterocyclyl ring; or
R⁶ is selected from the group consisting of H, alkyl, aminoalkyl, substituted or unsubstituted arylalkyl, substituted or unsubstituted cycloalkyl, alkoxyalkyl, hydroxyalkyl, substituted or unsubstituted heteroarylalkyl, substituted or unsubstituted aryl, substituted or unsubstituted heteroaryl, acyl, carboxy, alkoxycarbonyl, and substituted or unsubstituted aminocarbonyl.

In another embodiment of the compounds of formula (II), L² is a covalent bond, —C(O)—, or —C(O)O—; L³ is CH₂; L⁴ is a covalent bond or CH₂; X is selected from the group consisting of H, methyl, substituted or unsubstituted phenyl, or substituted or unsubstituted benzyl; Y is selected from the group consisting of substituted or unsubstituted phenyl, substituted or unsubstituted pyridyl, and substituted or unsubstituted imidazolyl, and substituted or unsubstituted 5,6,7,8,-tetrahydroquinolin-8-yl; R¹ is H or methyl; R⁵ is H; or R⁵ and R¹, together with the nitrogen atoms to which they are each attached, form a substituted or unsubstituted 5-membered or 6-membered heterocyclyl ring; and R⁶ is selected from the group consisting of H, methyl, substituted or unsubstituted imidazolylalkyl, and substituted or unsubstituted pyridylalkyl.

In another embodiment of the compounds of formula (II), L² is a covalent bond, —C(O)—, or —C(O)O—; L³ is C(R⁴)₂; L⁴ is a covalent bond or C(R⁴)₂; X is selected from the group consisting of H, alkyl, substituted or unsubstituted aryl, or substituted or unsubstituted arylkyl; Y is selected from the group consisting of substituted or unsubstituted aryl, substituted or unsubstituted cycloalkyl, and substituted or unsubstituted heteroaryl; R¹ is H or alkyl; R⁵ is H or alkyl; or R⁵ and R¹, together with the nitrogen atoms to which they are each attached, form a substituted or unsubstituted heterocyclyl ring; and R⁶ is selected from the group consisting of H, alkyl, and substituted and unsubstituted heteroarylalkyl.

In another embodiment of the compounds of formula (II), L² is a covalent bond.

In another embodiment of the compounds of formula (II), L² is —S(O)₂—.

In another embodiment of the compounds of formula (II), L² is —S(O)—.

In another embodiment of the compounds of formula (II), L² is —C(O)—.

In another embodiment of the compounds of formula (II), L² is —C(O)O—.

In another embodiment of the compounds of formula (II), L² is —C(R⁴)₂—, wherein R⁴ is as defined herein.

In another embodiment of the compounds of formula (II), L² is —C(R⁴)═, wherein R⁴ is as defined herein.

In another embodiment of the compounds of formula (II), L³ is —C(O)—.

In another embodiment of the compounds of formula (II), L³ is —C(R⁴)₂—, wherein R⁴ is as defined herein.

In another embodiment of the compounds of formula (II), L³ is —S(O)₂—.

In another embodiment of the compounds of formula (II), L³ is —S(O)—.

In another embodiment of the compounds of formula (II), L³ is CH₂—.

In another embodiment of the compounds of formula (II), L⁴ is —C(O)—.

In another embodiment of the compounds of formula (II), L⁴ is —C(R⁴)₂—, wherein R⁴ is as defined herein.

In another embodiment of the compounds of formula (II), L⁴ is —S(O)₂—.

In another embodiment of the compounds of formula (II), L⁴ is —S(O)—. In another embodiment of the compounds of formula (II), L⁴ is CH₂.

In another embodiment of the compounds of formula (II), R¹ is H.

In another embodiment of the compounds of formula (II), R¹ is alkyl.

In another embodiment of the compounds of formula (II), R¹ is alkenyl.

In another embodiment of the compounds of formula (II), R¹ is substituted or unsubstituted aminoalkyl.

In another embodiment of the compounds of formula (II), R¹ is substituted or unsubstituted arylalkyl.

In another embodiment of the compounds of formula (II), R¹ is substituted or unsubstituted —S(O)₂-aryl.

In another embodiment of the compounds of formula (II), R¹ is substituted or unsubstituted —S(O)₂-heteroaryl.

In another embodiment of the compounds of formula (II), R¹ is substituted or unsubstituted heteroarylalkyl.

In another embodiment of the compounds of formula (II), R¹ is substituted or unsubstituted cycloalkyl.

In another embodiment of the compounds of formula (II), R¹ is substituted or unsubstituted aryl, substituted or unsubstituted heteroaryl.

In another embodiment of the compounds of formula (II), R¹ is acyl.

In another embodiment of the compounds of formula (II), R¹ is carboxy.

In another embodiment of the compounds of formula (II), R¹ is alkoxycarbonyl.

In another embodiment of the compounds of formula (II), R¹ is substituted or unsubstituted aminocarbonyl.

In another embodiment of the compounds of formula (II), R¹ is methyl.

In another embodiment of the compounds of formula (II), R⁵ is H.

In another embodiment of the compounds of formula (II), R⁵ is substituted or unsubstituted heteroarylalkyl.

In another embodiment of the compounds of formula (II), R⁵ is alkyl.

In another embodiment of the compounds of formula (II), R⁵ is substituted or unsubstituted aminoalkyl.

In another embodiment of the compounds of formula (II), R⁵ is substituted or unsubstituted cycloalkyl.

In another embodiment of the compounds of formula (II), R⁵ is substituted or unsubstituted arylalkyl.

In another embodiment of the compounds of formula (II), R⁵ is substituted or unsubstituted aryl.

In another embodiment of the compounds of formula (II), R⁵ is substituted or unsubstituted heteroaryl.

In another embodiment of the compounds of formula (II), R⁵ is acyl.

In another embodiment of the compounds of formula (II), R⁵ is carboxy.

In another embodiment of the compounds of formula (II), R⁵ is alkoxycarbonyl.

In another embodiment of the compounds of formula (II), R⁵ is aminocarbonyl.

In another embodiment of the compounds of formula (II), R⁵ and R¹, together with the nitrogen atoms to which they are each attached, form a substituted or unsubstituted heterocyclyl ring.

In another embodiment of the compounds of formula (II), R⁵ and R¹, together with the nitrogen atoms to which they are each attached, form a substituted or unsubstituted 5-membered or 6-membered heterocyclyl ring.

In another embodiment of the compounds of formula (II), L² is a covalent bond and X is H.

In another embodiment of the compounds of formula (II), L² is a covalent bond and X is substituted or unsubstituted aryl.

In another embodiment of the compounds of formula (II), L² is a covalent bond and X is substituted or unsubstituted heteroaryl.

In another embodiment of the compounds of formula (II), L² is a covalent bond and X is substituted or unsubstituted phenyl.

In another embodiment of the compounds of formula (II), X is H.

In another embodiment of the compounds of formula (II), X is alkyl.

In another embodiment of the compounds of formula (II), X is substituted or unsubstituted aryl.

In another embodiment of the compounds of formula (II), X is substituted or unsubstituted heteroaryl.

In another embodiment of the compounds of formula (II), X is substituted or unsubstituted heterocyclyl.

In another embodiment of the compounds of formula (II), X is substituted or unsubstituted cycloalkyl.

In another embodiment of the compounds of formula (II), X is substituted or unsubstituted amino.

In another embodiment of the compounds of formula (II), X is substituted or unsubstituted amido.

In another embodiment of the compounds of formula (II), L² is —C(O)— and X is alkyl.

In another embodiment of the compounds of formula (II), L² is —C(O)— and X is methyl.

In another embodiment of the compounds of formula (II), L² is —C(O)O— and X is alkyl.

In another embodiment of the compounds of formula (II), L² is —C(O)O— and X is methyl.

In another embodiment of the compounds of formula (II), L² is —C(O)O— and X is arylalkyl.

In another embodiment of the compounds of formula (II), L² is —C(O)O— and X is heteroarylalkyl.

In another embodiment of the compounds of formula (II), L² is —C(O)O— and X is benzyl.

In another embodiment of the compounds of formula (II), L² is a covalent bond, X is H, and R⁵ is H.

In another embodiment of the compounds of formula (II), L² is a covalent bond, X is H, and R⁵ is alkyl.

In another embodiment of the compounds of formula (II), L² is a covalent bond, X is H, and R⁵ and R¹, together with the nitrogen atoms to which they are each attached, form a substituted or unsubstituted heterocyclyl ring.

In another embodiment of the compounds of formula (II), L² is a covalent bond, X is H, and R⁵ and R¹, together with the nitrogen atoms to which they are each attached, form a substituted or unsubstituted 5-membered or 6-membered heterocyclyl ring.

In another embodiment of the compounds of formula (II), L² is a covalent bond, X is substituted or unsubstituted aryl, and R¹ is H.

In another embodiment of the compounds of formula (II), L² is a covalent bond, X is substituted or unsubstituted aryl, and R⁵ is alkyl.

In another embodiment of the compounds of formula (II), L² is a covalent bond, X is substituted or unsubstituted aryl, and R⁵ and R¹, together with the nitrogen atoms to which they are each attached, form a substituted or unsubstituted heterocyclyl ring.

In another embodiment of the compounds of formula (II), L² is a covalent bond, X is substituted or unsubstituted aryl, and R⁵ and R¹, together with the nitrogen atoms to which they are each attached, form a substituted or unsubstituted 5-membered or 6-membered heterocyclyl ring.

In another embodiment of the compounds of formula (II), L² is a covalent bond, X is substituted or unsubstituted heteroaryl, and R⁵ is H.

In another embodiment of the compounds of formula (II), L² is a covalent bond, X is substituted or unsubstituted heteroaryl, and R⁵ is alkyl.

In another embodiment of the compounds of formula (II), L² is a covalent bond, X is substituted or unsubstituted heteroaryl, and R⁵ and R¹, together with the nitrogen atoms to which they are each attached, form a substituted or unsubstituted heterocyclyl ring.

In another embodiment of the compounds of formula (II), L² is a covalent bond, X is substituted or unsubstituted heteroaryl, and R⁵ and R¹, together with the nitrogen atoms to which they are each attached, form a substituted or unsubstituted 5-membered or 6-membered heterocyclyl ring.

In another embodiment of the compounds of formula (II), L² is a covalent bond, X is substituted or unsubstituted phenyl, and R⁵ is H.

In another embodiment of the compounds of formula (II), L² is a covalent bond, X is substituted or unsubstituted phenyl, and R⁵ is alkyl.

In another embodiment of the compounds of formula (II), L² is a covalent bond, X is substituted or unsubstituted phenyl, and R⁵ and R¹, together with the nitrogen atoms to which they are each attached, form a substituted or unsubstituted heterocyclyl ring.

In another embodiment of the compounds of formula (II), L² is a covalent bond, X is substituted or unsubstituted phenyl, and R⁵ and R¹, together with the nitrogen atoms to which they are each attached, form a substituted or unsubstituted 5-membered or 6-membered heterocyclyl ring.

In another embodiment of the compounds of formula (II), L² is —C(O)—, X is alkyl, and R⁵ is H.

In another embodiment of the compounds of formula (II), L² is —C(O)—, X is alkyl, and R⁵ is alkyl.

In another embodiment of the compounds of formula (II), L² is —C(O)—, X is alkyl, and R⁵ and R¹, together with the nitrogen atoms to which they are each attached, form a substituted or unsubstituted heterocyclyl ring.

In another embodiment of the compounds of formula (II), L² is —C(O)—, X is alkyl, and R⁵ and R¹, together with the nitrogen atoms to which they are each attached, form a substituted or unsubstituted 5-membered or 6-membered heterocyclyl ring.

In another embodiment of the compounds of formula (II), L² is —C(O)—, X is methyl, and R⁵ is H.

In another embodiment of the compounds of formula (II), L² is —C(O)—, X is methyl, and R⁵ is alkyl.

In another embodiment of the compounds of formula (II), L² is —C(O)—, X is methyl, and R⁵ and R¹, together with the nitrogen atoms to which they are each attached, form a substituted or unsubstituted heterocyclyl ring.

In another embodiment of the compounds of formula (II), L² is —C(O)—, X is methyl, and R⁵ and R¹, together with the nitrogen atoms to which they are each attached, form a substituted or unsubstituted 5-membered or 6-membered heterocyclyl ring.

In another embodiment of the compounds of formula (II), L² is —C(O)O—, X is alkyl, and R⁵ is H.

In another embodiment of the compounds of formula (II), L² is —C(O)O—, X is alkyl, and R⁵ is alkyl.

In another embodiment of the compounds of formula (II), L² is —C(O)O—, X is alkyl, and R⁵ and R¹, together with the nitrogen atoms to which they are each attached, form a substituted or unsubstituted heterocyclyl ring.

In another embodiment of the compounds of formula (II), L² is —C(O)O—, X is alkyl, and R⁵ and R¹, together with the nitrogen atoms to which they are each attached, form a substituted or unsubstituted 5-membered or 6-membered heterocyclyl ring.

In another embodiment of the compounds of formula (II), L² is —C(O)O—, X is methyl, and R⁵ is H.

In another embodiment of the compounds of formula (II), L² is —C(O)O—, X is methyl, and R⁵ is alkyl.

In another embodiment of the compounds of formula (II), L² is —C(O)O—, X is methyl, and R⁵ and R¹, together with the nitrogen atoms to which they are each attached, form a substituted or unsubstituted heterocyclyl ring.

In another embodiment of the compounds of formula (II), L² is —C(O)O—, X is methyl, and R⁵ and R¹, together with the nitrogen atoms to which they are each attached, form a substituted or unsubstituted 5-membered or 6-membered heterocyclyl ring.

In another embodiment of the compounds of formula (II), L² is —C(O)O—, X is substituted or unsubstituted arylalkyl, and R⁵ is H.

In another embodiment of the compounds of formula (II), L² is —C(O)O—, X is substituted or unsubstituted arylalkyl, and R⁵ is alkyl.

In another embodiment of the compounds of formula (II), L² is —C(O)O—, X is substituted or unsubstituted arylalkyl, and R⁵ and R¹, together with the nitrogen atoms to which they are each attached, form a substituted or unsubstituted heterocyclyl ring.

In another embodiment of the compounds of formula (II), L² is —C(O)O—, X is substituted or unsubstituted arylalkyl, and R⁵ and R¹, together with the nitrogen atoms to which they are each attached, form a substituted or unsubstituted 5-membered or 6-membered heterocyclyl ring.

In another embodiment of the compounds of formula (II), L² is —C(O)O—, X is substituted or unsubstituted heteroarylalkyl, and R⁵ is H.

In another embodiment of the compounds of formula (II), L² is —C(O)O—, X is substituted or unsubstituted heteroarylalkyl, and R⁵ is alkyl.

In another embodiment of the compounds of formula (II), L² is —C(O)O—, X is substituted or unsubstituted heteroarylalkyl, and R⁵ and R¹, together with the nitrogen atoms to which they are each attached, form a substituted or unsubstituted heterocyclyl ring.

In another embodiment of the compounds of formula (II), L² is —C(O)O—, X is substituted or unsubstituted heteroarylalkyl, and R⁵ and R¹, together with the nitrogen atoms to which they are each attached, form a substituted or unsubstituted 5-membered or 6-membered heterocyclyl ring.

In another embodiment of the compounds of formula (II), L² is —C(O)O—, X is substituted or unsubstituted benzyl, and R⁵ is H.

In another embodiment of the compounds of formula (II), L² is —C(O)O—, X is substituted or unsubstituted benzyl, and R⁵ is alkyl.

In another embodiment of the compounds of formula (II), L² is —C(O)O—, X is substituted or unsubstituted benzyl, and R⁵ and R¹, together with the nitrogen atoms to which they are each attached, form a substituted or unsubstituted heterocyclyl ring.

In another embodiment of the compounds of formula (II), L² is —C(O)O—, X is substituted or unsubstituted benzyl, and R⁵ and R¹, together with the nitrogen atoms to which they are each attached, form a substituted or unsubstituted 5-membered or 6-membered heterocyclyl ring.

In another embodiment of the compounds of formula (II), L³ is C(R⁴)₂ and L⁴ is C(R⁴)₂, wherein R⁴ is as defined herein.

In another embodiment of the compounds of formula (II), L³ is C(R⁴)₂ and L⁴ is CH₂, wherein R⁴ is as defined herein.

In another embodiment of the compounds of formula (II), L³ is C(R⁴)₂ and L⁴ is —C(O)—, wherein R⁴ is as defined herein.

In another embodiment of the compounds of formula (II), L³ is C(R⁴)₂ and L⁴ is a covalent bond, wherein R⁴ is as defined herein.

In another embodiment of the compounds of formula (II), L³ is CH₂ and L⁴ is C(R⁴)₂, wherein R⁴ is as defined herein.

In another embodiment of the compounds of formula (II), L³ is CH₂ and L⁴ is CH₂.

In another embodiment of the compounds of formula (II), L³ is C(R⁴)₂ and L⁴ is C═O, wherein R⁴ is as defined herein.

In another embodiment of the compounds of formula (II), L³ is CH₂ and L⁴ is a covalent bond, wherein R⁴ is as defined herein.

In another embodiment of the compounds of formula (II), Y is substituted or unsubstituted heteroaryl.

In another embodiment of the compounds of formula (II), Y is substituted or unsubstituted aryl.

In another embodiment of the compounds of formula (II), Y is hydroxyalkyl.

In another embodiment of the compounds of formula (II), Y is alkoxyalkyl.

In another embodiment of the compounds of formula (II), Y is aminoalkyl.

In another embodiment of the compounds of formula (II), Y is substituted or unsubstituted cycloalkyl.

In another embodiment of the compounds of formula (II), Y is substituted or unsubstituted arylalkyl.

In another embodiment of the compounds of formula (II), Y is substituted or unsubstituted —C(O)-heterocyclyl.

In another embodiment of the compounds of formula (II), Y is substituted or unsubstituted amino.

In another embodiment of the compounds of formula (II), Y is substituted or unsubstituted arylalkylamino.

In another embodiment of the compounds of formula (II), Y is substituted or unsubstituted heteroarylalkylamino.

In another embodiment of the compounds of formula (II), Y is substituted or unsubstituted aminoalkyl.

In another embodiment of the compounds of formula (II), Y is substituted or unsubstituted amido.

In another embodiment of the compounds of formula (II), Y is heterocyclyl.

In another embodiment of the compounds of formula (II), Y is alkyl.

In another embodiment of the compounds of formula (II), Y is substituted or unsubstituted phenyl.

In another embodiment of the compounds of formula (II), Y is substituted or unsubstituted pyridyl.

In another embodiment of the compounds of formula (II), Y is substituted or unsubstituted imidazolyl.

In another embodiment of the compounds of formula (II), Y is substituted or unsubstituted 5,6,7,8,-tetrahydroquinolin-8-yl.

In another embodiment of the compounds of formula (II), R⁶ is H.

In another embodiment of the compounds of formula (II), R⁶ is alkyl.

In another embodiment of the compounds of formula (II), R⁶ is substituted or unsubstituted aminoalkyl.

In another embodiment of the compounds of formula (II), R⁶ is substituted or unsubstituted arylalkyl.

In another embodiment of the compounds of formula (II), R⁶ is substituted or unsubstituted cycloalkyl.

In another embodiment of the compounds of formula (II), R⁶ is alkoxyalkyl.

In another embodiment of the compounds of formula (II), R⁶ is hydroxyalkyl.

In another embodiment of the compounds of formula (II), R⁶ is substituted or unsubstituted heteroarylalkyl.

In another embodiment of the compounds of formula (II), R⁶ is substituted or unsubstituted aryl.

In another embodiment of the compounds of formula (II), R⁶ is substituted or unsubstituted heteroaryl.

In another embodiment of the compounds of formula (II), R⁶ is acyl.

In another embodiment of the compounds of formula (II), R⁶ is carboxy.

In another embodiment of the compounds of formula (II), R⁶ is alkoxycarbonyl.

In another embodiment of the compounds of formula (II), R⁶ is substituted or unsubstituted aminocarbonyl.

In another embodiment of the compounds of formula (II), R⁶ is substituted or unsubstituted C₁-C₅ alkyl.

In another embodiment of the compounds of formula (II), R⁶ is methyl.

In another embodiment of the compounds of formula (II), R⁶ is substituted or unsubstituted imidazolylalkyl.

In another embodiment of the compounds of formula (II), R⁶ is pyridylalkyl.

In another embodiment of the compounds of formula (II), L³ is C(R⁴)₂ and L⁴ is C(R⁴)₂, wherein R⁴ is as defined herein, and Y is substituted or unsubstituted heteroaryl.

In another embodiment of the compounds of formula (II), L³ is C(R⁴)₂ and L⁴ is C(R⁴)₂, wherein R⁴ is as defined herein, and Y is substituted or unsubstituted imidazolyl.

In another embodiment of the compounds of formula (II), L³ is C(R⁴)₂ and L⁴ is C(R⁴)₂, wherein R⁴ is as defined herein, and Y is substituted or unsubstituted pyridyl.

In another embodiment of the compounds of formula (II), L³ is C(R⁴)₂ and L⁴ is C(R⁴)₂, wherein R⁴ is as defined herein, and Y is substituted or unsubstituted aryl.

In another embodiment of the compounds of formula (II), L³ is C(R⁴)₂ and L⁴ is C(R⁴)₂, wherein R⁴ is as defined herein, and Y is substituted or unsubstituted phenyl.

In another embodiment of the compounds of formula (II), L³ is C(R⁴)₂ and L⁴ is C(R⁴)₂, wherein R⁴ is as defined herein, and Y is substituted or unsubstituted cycloalkyl.

In another embodiment of the compounds of formula (II), L³ is C(R⁴)₂ and L⁴ is C(R⁴)₂, wherein R⁴ is as defined herein, and Y is substituted or unsubstituted 5,6,7,8,-tetrahydroquinolin-8-yl.

In another embodiment of the compounds of formula (II), L³ is CH₂ and L⁴ is C(R⁴)₂, wherein R⁴ is as defined herein, and Y is substituted or unsubstituted heteroaryl.

In another embodiment of the compounds of formula (II), L³ is CH₂ and L⁴ is C(R⁴)₂, wherein R⁴ is as defined herein, and Y is substituted or unsubstituted imidazolyl.

In another embodiment of the compounds of formula (II), L³ is CH₂ and L⁴ is C(R⁴)₂, wherein R⁴ is as defined herein, and Y is substituted or unsubstituted pyridyl.

In another embodiment of the compounds of formula (II), L³ is CH₂ and L⁴ is C(R⁴)₂, wherein R⁴ is as defined herein, and Y is substituted or unsubstituted aryl.

In another embodiment of the compounds of formula (II), L³ is CH₂ and L⁴ is C(R⁴)₂, wherein R⁴ is as defined herein, and Y is substituted or unsubstituted phenyl.

In another embodiment of the compounds of formula (II), L³ is CH₂ and L⁴ is C(R⁴)₂, wherein R⁴ is as defined herein, and Y is substituted or unsubstituted cycloalkyl.

In another embodiment of the compounds of formula (II), L³ is CH₂ and L⁴ is C(R⁴)₂, wherein R⁴ is as defined herein, and Y is substituted or unsubstituted 5,6,7,8,-tetrahydroquinolin-8-yl.

In another embodiment of the compounds of formula (II), L³ is C(R⁴)₂ and L⁴ is CH₂, wherein R⁴ is as defined herein, and Y is substituted or unsubstituted heteroaryl.

In another embodiment of the compounds of formula (II), L³ is C(R⁴)₂ and L⁴ is CH₂, wherein R⁴ is as defined herein, and Y is substituted or unsubstituted imidazolyl.

In another embodiment of the compounds of formula (II), L³ is C(R⁴)₂ and L⁴ is CH₂, wherein R⁴ is as defined herein, and Y is substituted or unsubstituted pyridyl.

In another embodiment of the compounds of formula (II), L³ is C(R⁴)₂ and L⁴ is CH₂, wherein R⁴ is as defined herein, and Y is substituted or unsubstituted aryl.

In another embodiment of the compounds of formula (II), L³ is C(R⁴)₂ and L⁴ is CH₂, wherein R⁴ is as defined herein, and Y is substituted or unsubstituted phenyl.

In another embodiment of the compounds of formula (II), L³ is C(R⁴)₂ and L⁴ is CH₂, wherein R⁴ is as defined herein, and Y is substituted or unsubstituted cycloalkyl.

In another embodiment of the compounds of formula (II), L³ is C(R⁴)₂ and L⁴ is CH₂, wherein R⁴ is as defined herein, and Y is substituted or unsubstituted 5,6,7,8,-tetrahydroquinolin-8-yl.

In another embodiment of the compounds of formula (II), L³ is CH₂ and L⁴ is CH₂, wherein R⁴ is as defined herein, and Y is substituted or unsubstituted heteroaryl.

In another embodiment of the compounds of formula (II), L³ is CH₂ and L⁴ is CH₂, wherein R⁴ is as defined herein, and Y is substituted or unsubstituted imidazolyl.

In another embodiment of the compounds of formula (II), L³ is CH₂ and L⁴ is CH₂, wherein R⁴ is as defined herein, and Y is substituted or unsubstituted pyridyl.

In another embodiment of the compounds of formula (II), L³ is CH₂ and L⁴ is CH₂, wherein R⁴ is as defined herein, and Y is substituted or unsubstituted aryl.

In another embodiment of the compounds of formula (II), L³ is CH₂ and L⁴ is CH₂, wherein R⁴ is as defined herein, and Y is substituted or unsubstituted phenyl.

In another embodiment of the compounds of formula (II), L³ is CH₂ and L⁴ is CH₂, wherein R⁴ is as defined herein, and Y is substituted or unsubstituted cycloalkyl.

In another embodiment of the compounds of formula (II), L³ is CH₂ and L⁴ is CH₂, wherein R⁴ is as defined herein, and Y is substituted or unsubstituted 5,6,7,8,-tetrahydroquinolin-8-yl.

In another embodiment of the compounds of formula (II), L³ is C(R⁴)₂ and L⁴ is C(R⁴)₂, wherein R⁴ is as defined herein, Y is substituted or unsubstituted heteroaryl, and R⁶ is H.

In another embodiment of the compounds of formula (II), L³ is C(R⁴)₂ and L⁴ is C(R⁴)₂, wherein R⁴ is as defined herein, Y is substituted or unsubstituted heteroaryl, and R⁶ is alkyl.

In another embodiment of the compounds of formula (II), L³ is C(R⁴)₂ and L⁴ is C(R⁴)₂, wherein R⁴ is as defined herein, Y is substituted or unsubstituted heteroaryl, and R⁶ is methyl.

In another embodiment of the compounds of formula (II), L³ is C(R⁴)₂ and L⁴ is C(R⁴)₂, wherein R⁴ is as defined herein, Y is substituted or unsubstituted heteroaryl, and R⁶ is arylalkyl.

In another embodiment of the compounds of formula (II), L³ is C(R⁴)₂ and L⁴ is C(R⁴)₂, wherein R⁴ is as defined herein, Y is substituted or unsubstituted heteroaryl, and R⁶ is heteroarylalkyl.

In another embodiment of the compounds of formula (II), L³ is C(R⁴)₂ and L⁴ is C(R⁴)₂, wherein R⁴ is as defined herein, Y is substituted or unsubstituted heteroaryl, and R⁶ is imidazolylalkyl.

In another embodiment of the compounds of formula (II), L³ is C(R⁴)₂ and L⁴ is C(R⁴)₂, wherein R⁴ is as defined herein, Y is substituted or unsubstituted heteroaryl, and R⁶ is pyridylalkyl.

In another embodiment of the compounds of formula (II), L³ is C(R⁴)₂ and L⁴ is C(R⁴)₂, wherein R⁴ is as defined herein, Y is substituted or unsubstituted imidazolyl, and R⁶ is H.

In another embodiment of the compounds of formula (II), L³ is C(R⁴)₂ and L⁴ is C(R⁴)₂, wherein R⁴ is as defined herein, Y is substituted or unsubstituted imidazolyl, and R⁶ is alkyl.

In another embodiment of the compounds of formula (II), L³ is C(R⁴)₂ and L⁴ is C(R⁴)₂, wherein R⁴ is as defined herein, Y is substituted or unsubstituted imidazolyl 1, and R⁶ is methyl.

In another embodiment of the compounds of formula (II), L³ is C(R⁴)₂ and L⁴ is C(R⁴)₂, wherein R⁴ is as defined herein, Y is substituted or unsubstituted imidazolyl, and R⁶ is arylalkyl.

In another embodiment of the compounds of formula (II), L³ is C(R⁴)₂ and L⁴ is C(R⁴)₂, wherein R⁴ is as defined herein, Y is substituted or unsubstituted imidazolyl, and R⁶ is heteroarylalkyl.

In another embodiment of the compounds of formula (II), L³ is C(R⁴)₂ and L⁴ is C(R⁴)₂, wherein R⁴ is as defined herein, Y is substituted or unsubstituted imidazolyl, and R⁶ is imidazolylalkyl.

In another embodiment of the compounds of formula (II), L³ is C(R⁴)₂ and L⁴ is C(R⁴)₂, wherein R⁴ is as defined herein, Y is substituted or unsubstituted imidazolyl, and R⁶ is pyridylalkyl.

In another embodiment of the compounds of formula (II), L³ is C(R⁴)₂ and L⁴ is C(R⁴)₂, wherein R⁴ is as defined herein, Y is substituted or unsubstituted pyridyl, and R⁶ is H.

In another embodiment of the compounds of formula (II), L³ is C(R⁴)₂ and L⁴ is C(R⁴)₂, wherein R⁴ is as defined herein, Y is substituted or unsubstituted pyridyl, and R⁶ is alkyl.

In another embodiment of the compounds of formula (II), L³ is C(R⁴)₂ and L⁴ is C(R⁴)₂, wherein R⁴ is as defined herein, Y is substituted or unsubstituted pyridyl, and R⁶ is methyl.

In another embodiment of the compounds of formula (II), L³ is C(R⁴)₂ and L⁴ is C(R⁴)₂, wherein R⁴ is as defined herein, Y is substituted or unsubstituted pyridyl, and R⁶ is arylalkyl.

In another embodiment of the compounds of formula (II), L³ is C(R⁴)₂ and L⁴ is C(R⁴)₂, wherein R⁴ is as defined herein, Y is substituted or unsubstituted pyridyl, and R⁶ is heteroarylalkyl.

In another embodiment of the compounds of formula (II), L³ is C(R⁴)₂ and L⁴ is C(R⁴)₂, wherein R⁴ is as defined herein, Y is substituted or unsubstituted pyridyl, and R⁶ is imidazolylalkyl.

In another embodiment of the compounds of formula (II), L³ is C(R⁴)₂ and L⁴ is C(R⁴)₂, wherein R⁴ is as defined herein, Y is substituted or unsubstituted pyridyl, and R⁶ is pyridylalkyl.

In another embodiment of the compounds of formula (II), L³ is C(R⁴)₂ and L⁴ is C(R⁴)₂, wherein R⁴ is as defined herein, Y is substituted or unsubstituted aryl, and R⁶ is H.

In another embodiment of the compounds of formula (II), L³ is C(R⁴)₂ and L⁴ is C(R⁴)₂, wherein R⁴ is as defined herein, Y is substituted or unsubstituted aryl, and R⁶ is alkyl.

In another embodiment of the compounds of formula (II), L³ is C(R⁴)₂ and L⁴ is C(R⁴)₂, wherein R⁴ is as defined herein, Y is substituted or unsubstituted aryl, and R⁶ is methyl.

In another embodiment of the compounds of formula (II), L³ is C(R⁴)₂ and L⁴ is C(R⁴)₂, wherein R⁴ is as defined herein, Y is substituted or unsubstituted aryl, and R⁶ is arylalkyl.

In another embodiment of the compounds of formula (II), L³ is C(R⁴)₂ and L⁴ is C(R⁴)₂, wherein R⁴ is as defined herein, Y is substituted or unsubstituted aryl, and R⁶ is heteroarylalkyl.

In another embodiment of the compounds of formula (II), L³ is C(R⁴)₂ and L⁴ is C(R⁴)₂, wherein R⁴ is as defined herein, Y is substituted or unsubstituted aryl, and R⁶ is imidazolylalkyl.

In another embodiment of the compounds of formula (II), L³ is C(R⁴)₂ and L⁴ is C(R⁴)₂, wherein R⁴ is as defined herein, Y is substituted or unsubstituted aryl, and R⁶ is pyridylalkyl.

In another embodiment of the compounds of formula (II), L³ is C(R⁴)₂ and L⁴ is C(R⁴)₂, wherein R⁴ is as defined herein, Y is substituted or unsubstituted phenyl, and R⁶ is H.

In another embodiment of the compounds of formula (II), L³ is C(R⁴)₂ and L⁴ is C(R⁴)₂, wherein R⁴ is as defined herein, Y is substituted or unsubstituted phenyl, and R⁶ is alkyl.

In another embodiment of the compounds of formula (II), L³ is C(R⁴)₂ and L⁴ is C(R⁴)₂, wherein R⁴ is as defined herein, Y is substituted or unsubstituted phenyl, and R⁶ is methyl.

In another embodiment of the compounds of formula (II), L³ is C(R⁴)₂ and L⁴ is C(R⁴)₂, wherein R⁴ is as defined herein, Y is substituted or unsubstituted phenyl, and R⁶ is arylalkyl.

In another embodiment of the compounds of formula (II), L³ is C(R⁴)₂ and L⁴ is C(R⁴)₂, wherein R⁴ is as defined herein, Y is substituted or unsubstituted phenyl, and R⁶ is heteroarylalkyl.

In another embodiment of the compounds of formula (II), L³ is C(R⁴)₂ and L⁴ is C(R⁴)₂, wherein R⁴ is as defined herein, Y is substituted or unsubstituted phenyl, and R⁶ is imidazolylalkyl.

In another embodiment of the compounds of formula (II), L³ is C(R⁴)₂ and L⁴ is C(R⁴)₂, wherein R⁴ is as defined herein, Y is substituted or unsubstituted phenyl, and R⁶ is pyridylalkyl.

In another embodiment of the compounds of formula (II), L³ is C(R⁴)₂ and L⁴ is C(R⁴)₂, wherein R⁴ is as defined herein, Y is substituted or unsubstituted cycloalkyl, and R⁶ is H.

In another embodiment of the compounds of formula (II), L³ is C(R⁴)₂ and L⁴ is C(R⁴)₂, wherein R⁴ is as defined herein, Y is substituted or unsubstituted cycloalkyl, and R⁶ is alkyl.

In another embodiment of the compounds of formula (II), L³ is C(R⁴)₂ and L⁴ is C(R⁴)₂, wherein R⁴ is as defined herein, Y is substituted or unsubstituted cycloalkyl, and R⁶ is methyl.

In another embodiment of the compounds of formula (II), L³ is C(R⁴)₂ and L⁴ is C(R⁴)₂, wherein R⁴ is as defined herein, Y is substituted or unsubstituted cycloalkyl, and R⁶ is arylalkyl.

In another embodiment of the compounds of formula (II), L³ is C(R⁴)₂ and L⁴ is C(R⁴)₂, wherein R⁴ is as defined herein, Y is substituted or unsubstituted cycloalkyl, and R⁶ is heteroarylalkyl.

In another embodiment of the compounds of formula (II), L³ is C(R⁴)₂ and L⁴ is C(R⁴)₂, wherein R⁴ is as defined herein, Y is substituted or unsubstituted cycloalkyl, and R⁶ is imidazolylalkyl.

In another embodiment of the compounds of formula (II), L³ is C(R⁴)₂ and L⁴ is C(R⁴)₂, wherein R⁴ is as defined herein, Y is substituted or unsubstituted cycloalkyl, and R⁶ is pyridylalkyl.

In another embodiment of the compounds of formula (II), L³ is C(R⁴)₂ and L⁴ is C(R⁴)₂, wherein R⁴ is as defined herein, Y is substituted or unsubstituted 5,6,7,8,-tetrahydroquinolin-8-yl, and R⁶ is H.

In another embodiment of the compounds of formula (II), L³ is C(R⁴)₂ and L⁴ is C(R⁴)₂, wherein R⁴ is as defined herein, Y is substituted or unsubstituted 5,6,7,8,-tetrahydroquinolin-8-yl, and R⁶ is alkyl.

In another embodiment of the compounds of formula (II), L³ is C(R⁴)₂ and L⁴ is C(R⁴)₂, wherein R⁴ is as defined herein, Y is substituted or unsubstituted 5,6,7,8,-tetrahydroquinolin-8-yl, and R⁶ is methyl.

In another embodiment of the compounds of formula (II), L³ is C(R⁴)₂ and L⁴ is C(R⁴)₂, wherein R⁴ is as defined herein, Y is substituted or unsubstituted 5,6,7,8,-tetrahydroquinolin-8-yl, and R⁶ is arylalkyl.

In another embodiment of the compounds of formula (II), L³ is C(R⁴)₂ and L⁴ is C(R⁴)₂, wherein R⁴ is as defined herein, Y is substituted or unsubstituted 5,6,7,8,-tetrahydroquinolin-8-yl, and R⁶ is heteroarylalkyl.

In another embodiment of the compounds of formula (II), L³ is C(R⁴)₂ and L⁴ is C(R⁴)₂, wherein R⁴ is as defined herein, Y is substituted or unsubstituted 5,6,7,8,-tetrahydroquinolin-8-yl, and R⁶ is imidazolylalkyl.

In another embodiment of the compounds of formula (II), L³ is C(R⁴)₂ and L⁴ is C(R⁴)₂, wherein R⁴ is as defined herein, Y is substituted or unsubstituted 5,6,7,8,-tetrahydroquinolin-8-yl, and R⁶ is pyridylalkyl.

In another embodiment of the compounds of formula (II), L³ is CH₂ and L⁴ is C(R⁴)₂, wherein R⁴ is as defined herein, Y is substituted or unsubstituted heteroaryl, and R⁶ is H.

In another embodiment of the compounds of formula (II), L³ is CH₂ and L⁴ is C(R⁴)₂, wherein R⁴ is as defined herein, Y is substituted or unsubstituted heteroaryl, and R⁶ is alkyl.

In another embodiment of the compounds of formula (II), L³ is CH₂ and L⁴ is C(R⁴)₂, wherein R⁴ is as defined herein, Y is substituted or unsubstituted heteroaryl, and R⁶ is methyl.

In another embodiment of the compounds of formula (II), L³ is CH₂ and L⁴ is C(R⁴)₂, wherein R⁴ is as defined herein, Y is substituted or unsubstituted heteroaryl, and R⁶ is arylalkyl.

In another embodiment of the compounds of formula (II), L³ is CH₂ and L⁴ is C(R⁴)₂, wherein R⁴ is as defined herein, Y is substituted or unsubstituted heteroaryl, and R⁶ is heteroarylalkyl.

In another embodiment of the compounds of formula (II), L³ is CH₂ and L⁴ is C(R⁴)₂, wherein R⁴ is as defined herein, Y is substituted or unsubstituted heteroaryl, and R⁶ is imidazolylalkyl.

In another embodiment of the compounds of formula (II), L³ is CH₂ and L⁴ is C(R⁴)₂, wherein R⁴ is as defined herein, Y is substituted or unsubstituted heteroaryl, and R⁶ is pyridylalkyl.

In another embodiment of the compounds of formula (II), L³ is CH₂ and L⁴ is C(R⁴)₂, wherein R⁴ is as defined herein, Y is substituted or unsubstituted imidazolyl, and R⁶ is H.

In another embodiment of the compounds of formula (II), L³ is CH₂ and L⁴ is C(R⁴)₂, wherein R⁴ is as defined herein, Y is substituted or unsubstituted imidazolyl, and R⁶ is alkyl.

In another embodiment of the compounds of formula (II), L³ is CH₂ and L⁴ is C(R⁴)₂, wherein R⁴ is as defined herein, Y is substituted or unsubstituted imidazolyl 1, and R⁶ is methyl.

In another embodiment of the compounds of formula (II), L³ is CH₂ and L⁴ is C(R⁴)₂, wherein R⁴ is as defined herein, Y is substituted or unsubstituted imidazolyl, and R⁶ is arylalkyl.

In another embodiment of the compounds of formula (II), L³ is CH₂ and L⁴ is C(R⁴)₂, wherein R⁴ is as defined herein, Y is substituted or unsubstituted imidazolyl, and R⁶ is heteroarylalkyl.

In another embodiment of the compounds of formula (II), L³ is CH₂ and L⁴ is C(R⁴)₂, wherein R⁴ is as defined herein, Y is substituted or unsubstituted imidazolyl, and R⁶ is imidazolylalkyl.

In another embodiment of the compounds of formula (II), L³ is CH₂ and L⁴ is C(R⁴)₂, wherein R⁴ is as defined herein, Y is substituted or unsubstituted imidazolyl, and R⁶ is pyridylalkyl.

In another embodiment of the compounds of formula (II), L³ is CH₂ and L⁴ is C(R⁴)₂, wherein R⁴ is as defined herein, Y is substituted or unsubstituted pyridyl, and R⁶ is H.

In another embodiment of the compounds of formula (II), L³ is CH₂ and L⁴ is C(R⁴)₂, wherein R⁴ is as defined herein, Y is substituted or unsubstituted pyridyl, and R⁶ is alkyl.

In another embodiment of the compounds of formula (II), L³ is CH₂ and L⁴ is C(R⁴)₂, wherein R⁴ is as defined herein, Y is substituted or unsubstituted pyridyl, and R⁶ is methyl.

In another embodiment of the compounds of formula (II), L³ is CH₂ and L⁴ is C(R⁴)₂, wherein R⁴ is as defined herein, Y is substituted or unsubstituted pyridyl, and R⁶ is arylalkyl.

In another embodiment of the compounds of formula (II), L³ is CH₂ and L⁴ is C(R⁴)₂, wherein R⁴ is as defined herein, Y is substituted or unsubstituted pyridyl, and R⁶ is heteroarylalkyl.

In another embodiment of the compounds of formula (II), L³ is CH₂ and L⁴ is C(R⁴)₂, wherein R⁴ is as defined herein, Y is substituted or unsubstituted pyridyl, and R⁶ is imidazolylalkyl.

In another embodiment of the compounds of formula (II), L³ is CH₂ and L⁴ is C(R⁴)₂, wherein R⁴ is as defined herein, Y is substituted or unsubstituted pyridyl, and R⁶ is pyridylalkyl.

In another embodiment of the compounds of formula (II), L³ is CH₂ and L⁴ is C(R⁴)₂, wherein R⁴ is as defined herein, Y is substituted or unsubstituted aryl, and R⁶ is H.

In another embodiment of the compounds of formula (II), L³ is CH₂ and L⁴ is C(R⁴)₂, wherein R⁴ is as defined herein, Y is substituted or unsubstituted aryl, and R⁶ is alkyl.

In another embodiment of the compounds of formula (II), L³ is CH₂ and L⁴ is C(R⁴)₂, wherein R⁴ is as defined herein, Y is substituted or unsubstituted aryl, and R⁶ is methyl.

In another embodiment of the compounds of formula (II), L³ is CH₂ and L⁴ is C(R⁴)₂, wherein R⁴ is as defined herein, Y is substituted or unsubstituted aryl, and R⁶ is arylalkyl.

In another embodiment of the compounds of formula (II), L³ is CH₂ and L⁴ is C(R⁴)₂, wherein R⁴ is as defined herein, Y is substituted or unsubstituted aryl, and R⁶ is heteroarylalkyl.

In another embodiment of the compounds of formula (II), L³ is CH₂ and L⁴ is C(R⁴)₂, wherein R⁴ is as defined herein, Y is substituted or unsubstituted aryl, and R⁶ is imidazolylalkyl.

In another embodiment of the compounds of formula (II), L³ is CH₂ and L⁴ is C(R⁴)₂, wherein R⁴ is as defined herein, Y is substituted or unsubstituted aryl, and R⁶ is pyridylalkyl.

In another embodiment of the compounds of formula (II), L³ is CH₂ and L⁴ is C(R⁴)₂, wherein R⁴ is as defined herein, Y is substituted or unsubstituted phenyl, and R⁶ is H.

In another embodiment of the compounds of formula (II), L³ is CH₂ and L⁴ is C(R⁴)₂, wherein R⁴ is as defined herein, Y is substituted or unsubstituted phenyl, and R⁶ is alkyl.

In another embodiment of the compounds of formula (II), L³ is CH₂ and L⁴ is C(R⁴)₂, wherein R⁴ is as defined herein, Y is substituted or unsubstituted phenyl, and R⁶ is methyl.

In another embodiment of the compounds of formula (II), L³ is CH₂ and L⁴ is C(R⁴)₂, wherein R⁴ is as defined herein, Y is substituted or unsubstituted phenyl, and R⁶ is arylalkyl.

In another embodiment of the compounds of formula (II), L³ is CH₂ and L⁴ is C(R⁴)₂, wherein R⁴ is as defined herein, Y is substituted or unsubstituted phenyl, and R⁶ is heteroarylalkyl.

In another embodiment of the compounds of formula (II), L³ is CH₂ and L⁴ is C(R⁴)₂, wherein R⁴ is as defined herein, Y is substituted or unsubstituted phenyl, and R⁶ is imidazolylalkyl.

In another embodiment of the compounds of formula (II), L³ is CH₂ and L⁴ is C(R⁴)₂, wherein R⁴ is as defined herein, Y is substituted or unsubstituted phenyl, and R⁶ is pyridylalkyl.

In another embodiment of the compounds of formula (II), L³ is CH₂ and L⁴ is C(R⁴)₂, wherein R⁴ is as defined herein, Y is substituted or unsubstituted cycloalkyl, and R⁶ is H.

In another embodiment of the compounds of formula (II), L³ is CH₂ and L⁴ is C(R⁴)₂, wherein R⁴ is as defined herein, Y is substituted or unsubstituted cycloalkyl, and R⁶ is alkyl.

In another embodiment of the compounds of formula (II), L³ is CH₂ and L⁴ is C(R⁴)₂, wherein R⁴ is as defined herein, Y is substituted or unsubstituted cycloalkyl, and R⁶ is methyl.

In another embodiment of the compounds of formula (II), L³ is CH₂ and L⁴ is C(R⁴)₂, wherein R⁴ is as defined herein, Y is substituted or unsubstituted cycloalkyl, and R⁶ is arylalkyl.

In another embodiment of the compounds of formula (II), L³ is CH₂ and L⁴ is C(R⁴)₂, wherein R⁴ is as defined herein, Y is substituted or unsubstituted cycloalkyl, and R⁶ is heteroarylalkyl.

In another embodiment of the compounds of formula (II), L³ is CH₂ and L⁴ is C(R⁴)₂, wherein R⁴ is as defined herein, Y is substituted or unsubstituted cycloalkyl, and R⁶ is imidazolylalkyl.

In another embodiment of the compounds of formula (II), L³ is CH₂ and L⁴ is C(R⁴)₂, wherein R⁴ is as defined herein, Y is substituted or unsubstituted cycloalkyl, and R⁶ is pyridylalkyl.

In another embodiment of the compounds of formula (II), L³ is CH₂ and L⁴ is C(R⁴)₂, wherein R⁴ is as defined herein, Y is substituted or unsubstituted 5,6,7,8,-tetrahydroquinolin-8-yl, and R⁶ is H.

In another embodiment of the compounds of formula (II), L³ is CH₂ and L⁴ is C(R⁴)₂, wherein R⁴ is as defined herein, Y is substituted or unsubstituted 5,6,7,8,-tetrahydroquinolin-8-yl, and R⁶ is alkyl.

In another embodiment of the compounds of formula (II), L³ is CH₂ and L⁴ is C(R⁴)₂, wherein R⁴ is as defined herein, Y is substituted or unsubstituted 5,6,7,8,-tetrahydroquinolin-8-yl, and R⁶ is methyl.

In another embodiment of the compounds of formula (II), L³ is CH₂ and L⁴ is C(R⁴)₂, wherein R⁴ is as defined herein, Y is substituted or unsubstituted 5,6,7,8,-tetrahydroquinolin-8-yl, and R⁶ is arylalkyl.

In another embodiment of the compounds of formula (II), L³ is CH₂ and L⁴ is C(R⁴)₂, wherein R⁴ is as defined herein, Y is substituted or unsubstituted 5,6,7,8,-tetrahydroquinolin-8-yl, and R⁶ is heteroarylalkyl.

In another embodiment of the compounds of formula (II), L³ is CH₂ and L⁴ is C(R⁴)₂, wherein R⁴ is as defined herein, Y is substituted or unsubstituted 5,6,7,8,-tetrahydroquinolin-8-yl, and R⁶ is imidazolylalkyl.

In another embodiment of the compounds of formula (II), L³ is CH₂ and L⁴ is C(R⁴)₂, wherein R⁴ is as defined herein, Y is substituted or unsubstituted 5,6,7,8,-tetrahydroquinolin-8-yl, and R⁶ is pyridylalkyl.

In another embodiment of the compounds of formula (II), L³ is C(R⁴)₂ and L⁴ is CH₂, wherein R⁴ is as defined herein, Y is substituted or unsubstituted heteroaryl, and R⁶ is H.

In another embodiment of the compounds of formula (II), L³ is C(R⁴)₂ and L⁴ is CH₂, wherein R⁴ is as defined herein, Y is substituted or unsubstituted heteroaryl, and R⁶ is alkyl.

In another embodiment of the compounds of formula (II), L³ is C(R⁴)₂ and L⁴ is CH₂, wherein R⁴ is as defined herein, Y is substituted or unsubstituted heteroaryl, and R⁶ is methyl.

In another embodiment of the compounds of formula (II), L³ is C(R⁴)₂ and L⁴ is CH₂, wherein R⁴ is as defined herein, Y is substituted or unsubstituted heteroaryl, and R⁶ is arylalkyl.

In another embodiment of the compounds of formula (II), L³ is C(R⁴)₂ and L⁴ is CH₂, wherein R⁴ is as defined herein, Y is substituted or unsubstituted heteroaryl, and R⁶ is heteroarylalkyl.

In another embodiment of the compounds of formula (II), L³ is C(R⁴)₂ and L⁴ is CH₂, wherein R⁴ is as defined herein, Y is substituted or unsubstituted heteroaryl, and R⁶ is imidazolylalkyl.

In another embodiment of the compounds of formula (II), L³ is C(R⁴)₂ and L⁴ is CH₂, wherein R⁴ is as defined herein, Y is substituted or unsubstituted heteroaryl, and R⁶ is pyridylalkyl.

In another embodiment of the compounds of formula (II), L³ is C(R⁴)₂ and L⁴ is CH₂, wherein R⁴ is as defined herein, Y is substituted or unsubstituted imidazolyl, and R¹ is H.

In another embodiment of the compounds of formula (II), L³ is C(R⁴)₂ and L⁴ is CH₂, wherein R⁴ is as defined herein, Y is substituted or unsubstituted imidazolyl, and R⁶ is alkyl.

In another embodiment of the compounds of formula (II), L³ is C(R⁴)₂ and L⁴ is CH₂, wherein R⁴ is as defined herein, Y is substituted or unsubstituted imidazolyl 1, and R⁶ is methyl.

In another embodiment of the compounds of formula (II), L³ is C(R⁴)₂ and L⁴ is CH₂, wherein R⁴ is as defined herein, Y is substituted or unsubstituted imidazolyl, and R⁶ is arylalkyl.

In another embodiment of the compounds of formula (II), L³ is C(R⁴)₂ and L⁴ is CH₂, wherein R⁴ is as defined herein, Y is substituted or unsubstituted imidazolyl, and R⁶ is heteroarylalkyl.

In another embodiment of the compounds of formula (II), L³ is C(R⁴)₂ and L⁴ is CH₂, wherein R⁴ is as defined herein, Y is substituted or unsubstituted imidazolyl, and R⁶ is imidazolylalkyl.

In another embodiment of the compounds of formula (II), L³ is C(R⁴)₂ and L⁴ is CH₂, wherein R⁴ is as defined herein, Y is substituted or unsubstituted imidazolyl, and R⁶ is pyridylalkyl.

In another embodiment of the compounds of formula (II), L³ is C(R⁴)₂ and L⁴ is CH₂, wherein R⁴ is as defined herein, Y is substituted or unsubstituted pyridyl, and R⁶ is H.

In another embodiment of the compounds of formula (II), L³ is C(R⁴)₂ and L⁴ is CH₂, wherein R⁴ is as defined herein, Y is substituted or unsubstituted pyridyl, and R⁶ is alkyl.

In another embodiment of the compounds of formula (II), L³ is C(R⁴)₂ and L⁴ is CH₂, wherein R⁴ is as defined herein, Y is substituted or unsubstituted pyridyl, and R⁶ is methyl.

In another embodiment of the compounds of formula (II), L³ is C(R⁴)₂ and L⁴ is CH₂, wherein R⁴ is as defined herein, Y is substituted or unsubstituted pyridyl, and R⁶ is arylalkyl.

In another embodiment of the compounds of formula (II), L³ is C(R⁴)₂ and L⁴ is CH₂, wherein R⁴ is as defined herein, Y is substituted or unsubstituted pyridyl, and R⁶ is heteroarylalkyl.

In another embodiment of the compounds of formula (II), L³ is C(R⁴)₂ and L⁴ is CH₂, wherein R⁴ is as defined herein, Y is substituted or unsubstituted pyridyl, and R⁶ is imidazolylalkyl.

In another embodiment of the compounds of formula (II), L³ is C(R⁴)₂ and L⁴ is CH₂, wherein R⁴ is as defined herein, Y is substituted or unsubstituted pyridyl, and R⁶ is pyridylalkyl.

In another embodiment of the compounds of formula (II), L³ is C(R⁴)₂ and L⁴ is CH₂, wherein R⁴ is as defined herein, Y is substituted or unsubstituted aryl, and R⁶ is H.

In another embodiment of the compounds of formula (II), L³ is C(R⁴)₂ and L⁴ is C(R⁴)₂, wherein R⁴ is as defined herein, Y is substituted or unsubstituted aryl, and R⁶ is alkyl.

In another embodiment of the compounds of formula (II), L³ is C(R⁴)₂ and L⁴ is CH₂, wherein R⁴ is as defined herein, Y is substituted or unsubstituted aryl, and R⁶ is methyl.

In another embodiment of the compounds of formula (II), L³ is C(R⁴)₂ and L⁴ is CH₂, wherein R⁴ is as defined herein, Y is substituted or unsubstituted aryl, and R⁶ is arylalkyl.

In another embodiment of the compounds of formula (II), L³ is C(R⁴)₂ and L⁴ is CH₂, wherein R⁴ is as defined herein, Y is substituted or unsubstituted aryl, and R⁶ is heteroarylalkyl.

In another embodiment of the compounds of formula (II), L³ is C(R⁴)₂ and L⁴ is CH₂, wherein R⁴ is as defined herein, Y is substituted or unsubstituted aryl, and R⁶ is imidazolylalkyl.

In another embodiment of the compounds of formula (II), L³ is C(R⁴)₂ and L⁴ is CH₂, wherein R⁴ is as defined herein, Y is substituted or unsubstituted aryl, and R⁶ is pyridylalkyl.

In another embodiment of the compounds of formula (II), L³ is C(R⁴)₂ and L⁴ is CH₂, wherein R⁴ is as defined herein, Y is substituted or unsubstituted phenyl, and R¹ is H.

In another embodiment of the compounds of formula (II), L³ is C(R⁴)₂ and L⁴ is CH₂, wherein R⁴ is as defined herein, Y is substituted or unsubstituted phenyl, and R⁶ is alkyl.

In another embodiment of the compounds of formula (II), L³ is C(R⁴)₂ and L⁴ is CH₂, wherein R⁴ is as defined herein, Y is substituted or unsubstituted phenyl, and R⁶ is methyl.

In another embodiment of the compounds of formula (II), L³ is C(R⁴)₂ and L⁴ is CH₂, wherein R⁴ is as defined herein, Y is substituted or unsubstituted phenyl, and R⁶ is arylalkyl.

In another embodiment of the compounds of formula (II), L³ is C(R⁴)₂ and L⁴ is CH₂, wherein R⁴ is as defined herein, Y is substituted or unsubstituted phenyl, and R⁶ is heteroarylalkyl.

In another embodiment of the compounds of formula (II), L³ is C(R⁴)₂ and L⁴ is CH₂, wherein R⁴ is as defined herein, Y is substituted or unsubstituted phenyl, and R⁶ is imidazolylalkyl.

In another embodiment of the compounds of formula (II), L³ is C(R⁴)₂ and L⁴ is CH₂, wherein R⁴ is as defined herein, Y is substituted or unsubstituted phenyl, and R⁶ is pyridylalkyl.

In another embodiment of the compounds of formula (II), L³ is C(R⁴)₂ and L⁴ is CH₂, wherein R⁴ is as defined herein, Y is substituted or unsubstituted cycloalkyl, and R⁶ is H.

In another embodiment of the compounds of formula (II), L³ is C(R⁴)₂ and L⁴ is CH₂, wherein R⁴ is as defined herein, Y is substituted or unsubstituted cycloalkyl, and R⁶ is alkyl.

In another embodiment of the compounds of formula (II), L³ is C(R⁴)₂ and L⁴ is CH₂, wherein R⁴ is as defined herein, Y is substituted or unsubstituted cycloalkyl, and R⁶ is methyl.

In another embodiment of the compounds of formula (II), L³ is C(R⁴)₂ and L⁴ is CH₂, wherein R⁴ is as defined herein, Y is substituted or unsubstituted cycloalkyl, and R⁶ is arylalkyl.

In another embodiment of the compounds of formula (II), L³ is C(R⁴)₂ and L⁴ is CH₂, wherein R⁴ is as defined herein, Y is substituted or unsubstituted cycloalkyl, and R⁶ is heteroarylalkyl.

In another embodiment of the compounds of formula (II), L³ is C(R⁴)₂ and L⁴ is CH₂, wherein R⁴ is as defined herein, Y is substituted or unsubstituted cycloalkyl, and R⁶ is imidazolylalkyl.

In another embodiment of the compounds of formula (II), L³ is C(R⁴)₂ and L⁴ is CH₂, wherein R⁴ is as defined herein, Y is substituted or unsubstituted cycloalkyl, and R⁶ is pyridylalkyl.

In another embodiment of the compounds of formula (II), L³ is C(R⁴)₂ and L⁴ is CH₂, wherein R⁴ is as defined herein, Y is substituted or unsubstituted 5,6,7,8,-tetrahydroquinolin-8-yl, and R⁶ is H.

In another embodiment of the compounds of formula (II), L³ is C(R⁴)₂ and L⁴ is CH₂, wherein R⁴ is as defined herein, Y is substituted or unsubstituted 5,6,7,8,-tetrahydroquinolin-8-yl, and R⁶ is alkyl.

In another embodiment of the compounds of formula (II), L³ is C(R⁴)₂ and L⁴ is CH₂, wherein R⁴ is as defined herein, Y is substituted or unsubstituted 5,6,7,8,-tetrahydroquinolin-8-yl, and R⁶ is methyl.

In another embodiment of the compounds of formula (II), L³ is C(R⁴)₂ and L⁴ is CH₂, wherein R⁴ is as defined herein, Y is substituted or unsubstituted 5,6,7,8,-tetrahydroquinolin-8-yl, and R⁶ is arylalkyl.

In another embodiment of the compounds of formula (II), L³ is C(R⁴)₂ and L⁴ is CH₂, wherein R⁴ is as defined herein, Y is substituted or unsubstituted 5,6,7,8,-tetrahydroquinolin-8-yl, and R⁶ is heteroarylalkyl.

In another embodiment of the compounds of formula (II), L³ is C(R⁴)₂ and L⁴ is CH₂, wherein R⁴ is as defined herein, Y is substituted or unsubstituted 5,6,7,8,-tetrahydroquinolin-8-yl, and R⁶ is imidazolylalkyl.

In another embodiment of the compounds of formula (II), L³ is C(R⁴)₂ and L⁴ is CH₂, wherein R⁴ is as defined herein, Y is substituted or unsubstituted 5,6,7,8,-tetrahydroquinolin-8-yl, and R⁶ is pyridylalkyl.

In another embodiment of the compounds of formula (II), L³ is CH₂ and L⁴ is CH₂, wherein R⁴ is as defined herein, Y is substituted or unsubstituted heteroaryl, and R⁶ is H.

In another embodiment of the compounds of formula (II), L³ is CH₂ and L⁴ is CH₂, wherein R⁴ is as defined herein, Y is substituted or unsubstituted heteroaryl, and R⁶ is alkyl.

In another embodiment of the compounds of formula (II), L³ is CH₂ and L⁴ is CH₂, wherein R⁴ is as defined herein, Y is substituted or unsubstituted heteroaryl, and R⁶ is methyl.

In another embodiment of the compounds of formula (II), L³ is CH₂ and L⁴ is CH₂, wherein R⁴ is as defined herein, Y is substituted or unsubstituted heteroaryl, and R⁶ is arylalkyl.

In another embodiment of the compounds of formula (II), L³ is CH₂ and L⁴ is CH₂, wherein R⁴ is as defined herein, Y is substituted or unsubstituted heteroaryl, and R⁶ is heteroarylalkyl.

In another embodiment of the compounds of formula (II), L³ is CH₂ and L⁴ is CH₂, wherein R⁴ is as defined herein, Y is substituted or unsubstituted heteroaryl, and R⁶ is imidazolylalkyl.

In another embodiment of the compounds of formula (II), L³ is CH₂ and L⁴ is CH₂, wherein R⁴ is as defined herein, Y is substituted or unsubstituted heteroaryl, and R⁶ is pyridylalkyl.

In another embodiment of the compounds of formula (II), L³ is CH₂ and L⁴ is CH₂, wherein R⁴ is as defined herein, Y is substituted or unsubstituted imidazolyl, and R⁶ is H.

In another embodiment of the compounds of formula (II), L³ is CH₂ and L⁴ is CH₂, wherein R⁴ is as defined herein, Y is substituted or unsubstituted imidazolyl, and R⁶ is alkyl.

In another embodiment of the compounds of formula (II), L³ is CH₂ and L⁴ is CH₂, wherein R⁴ is as defined herein, Y is substituted or unsubstituted imidazolyl 1, and R⁶ is methyl.

In another embodiment of the compounds of formula (II), L³ is CH₂ and L⁴ is CH₂, wherein R⁴ is as defined herein, Y is substituted or unsubstituted imidazolyl, and R⁶ is arylalkyl.

In another embodiment of the compounds of formula (II), L³ is CH₂ and L⁴ is CH₂, wherein R⁴ is as defined herein, Y is substituted or unsubstituted imidazolyl, and R⁶ is heteroarylalkyl.

In another embodiment of the compounds of formula (II), L³ is CH₂ and L⁴ is CH₂, wherein R⁴ is as defined herein, Y is substituted or unsubstituted imidazolyl, and R⁶ is imidazolylalkyl.

In another embodiment of the compounds of formula (II), L³ is CH₂ and L⁴ is CH₂, wherein R⁴ is as defined herein, Y is substituted or unsubstituted imidazolyl, and R⁶ is pyridylalkyl.

In another embodiment of the compounds of formula (II), L³ is CH₂ and L⁴ is CH₂, wherein R⁴ is as defined herein, Y is substituted or unsubstituted pyridyl, and R⁶ is H.

In another embodiment of the compounds of formula (II), L³ is CH₂ and L⁴ is CH₂, wherein R⁴ is as defined herein, Y is substituted or unsubstituted pyridyl, and R⁶ is alkyl.

In another embodiment of the compounds of formula (II), L³ is CH₂ and L⁴ is CH₂, wherein R⁴ is as defined herein, Y is substituted or unsubstituted pyridyl, and R⁶ is methyl.

In another embodiment of the compounds of formula (II), L³ is CH₂ and L⁴ is CH₂, wherein R⁴ is as defined herein, Y is substituted or unsubstituted pyridyl, and R⁶ is arylalkyl.

In another embodiment of the compounds of formula (II), L³ is CH₂ and L⁴ is CH₂, wherein R⁴ is as defined herein, Y is substituted or unsubstituted pyridyl, and R⁶ is heteroarylalkyl.

In another embodiment of the compounds of formula (II), L³ is CH₂ and L⁴ is CH₂, wherein R⁴ is as defined herein, Y is substituted or unsubstituted pyridyl, and R⁶ is imidazolylalkyl.

In another embodiment of the compounds of formula (II), L³ is CH₂ and L⁴ is CH₂, wherein R⁴ is as defined herein, Y is substituted or unsubstituted pyridyl, and R⁶ is pyridylalkyl.

In another embodiment of the compounds of formula (II), L³ is CH₂ and L⁴ is CH₂, wherein R⁴ is as defined herein, Y is substituted or unsubstituted aryl, and R⁶ is H.

In another embodiment of the compounds of formula (II), L³ is CH₂ and L⁴ is CH₂, wherein R⁴ is as defined herein, Y is substituted or unsubstituted aryl, and R⁶ is alkyl.

In another embodiment of the compounds of formula (II), L³ is CH₂ and L⁴ is CH₂, wherein R⁴ is as defined herein, Y is substituted or unsubstituted aryl, and R⁶ is methyl.

In another embodiment of the compounds of formula (II), L³ is CH₂ and L⁴ is CH₂, wherein R⁴ is as defined herein, Y is substituted or unsubstituted aryl, and R⁶ is arylalkyl.

In another embodiment of the compounds of formula (II), L³ is CH₂ and L⁴ is CH₂, wherein R⁴ is as defined herein, Y is substituted or unsubstituted aryl, and R⁶ is heteroarylalkyl.

In another embodiment of the compounds of formula (II), L³ is CH₂ and L⁴ is CH₂, wherein R⁴ is as defined herein, Y is substituted or unsubstituted aryl, and R⁶ is imidazolylalkyl.

In another embodiment of the compounds of formula (II), L³ is CH₂ and L⁴ is CH₂, wherein R⁴ is as defined herein, Y is substituted or unsubstituted aryl, and R⁶ is pyridylalkyl.

In another embodiment of the compounds of formula (II), L³ is CH₂ and L⁴ is CH₂, wherein R⁴ is as defined herein, Y is substituted or unsubstituted phenyl, and R⁶ is H.

In another embodiment of the compounds of formula (II), L³ is CH₂ and L⁴ is CH₂, wherein R⁴ is as defined herein, Y is substituted or unsubstituted phenyl, and R⁶ is alkyl.

In another embodiment of the compounds of formula (II), L³ is CH₂ and L⁴ is CH₂, wherein R⁴ is as defined herein, Y is substituted or unsubstituted phenyl, and R⁶ is methyl.

In another embodiment of the compounds of formula (II), L³ is CH₂ and L⁴ is CH₂, wherein R⁴ is as defined herein, Y is substituted or unsubstituted phenyl, and R⁶ is arylalkyl.

In another embodiment of the compounds of formula (II), L³ is CH₂ and L⁴ is CH₂, wherein R⁴ is as defined herein, Y is substituted or unsubstituted phenyl, and R⁶ is heteroarylalkyl.

In another embodiment of the compounds of formula (II), L³ is CH₂ and L⁴ is CH₂, wherein R⁴ is as defined herein, Y is substituted or unsubstituted phenyl, and R⁶ is imidazolylalkyl.

In another embodiment of the compounds of formula (II), L³ is CH₂ and L⁴ is CH₂, wherein R⁴ is as defined herein, Y is substituted or unsubstituted phenyl, and R⁶ is pyridylalkyl.

In another embodiment of the compounds of formula (II), L³ is CH₂ and L⁴ is CH₂, wherein R⁴ is as defined herein, Y is substituted or unsubstituted cycloalkyl, and R¹ is H.

In another embodiment of the compounds of formula (II), L³ is CH₂ and L⁴ is CH₂, wherein R⁴ is as defined herein, Y is substituted or unsubstituted cycloalkyl, and R⁶ is alkyl.

In another embodiment of the compounds of formula (II), L³ is CH₂ and L⁴ is CH₂, wherein R⁴ is as defined herein, Y is substituted or unsubstituted cycloalkyl, and R⁶ is methyl.

In another embodiment of the compounds of formula (II), L³ is CH₂ and L⁴ is CH₂, wherein R⁴ is as defined herein, Y is substituted or unsubstituted cycloalkyl, and R⁶ is arylalkyl.

In another embodiment of the compounds of formula (II), L³ is CH₂ and L⁴ is CH₂, wherein R⁴ is as defined herein, Y is substituted or unsubstituted cycloalkyl, and R⁶ is heteroarylalkyl.

In another embodiment of the compounds of formula (II), L³ is CH₂ and L⁴ is CH₂, wherein R⁴ is as defined herein, Y is substituted or unsubstituted cycloalkyl, and R⁶ is imidazolylalkyl.

In another embodiment of the compounds of formula (II), L³ is CH₂ and L⁴ is CH₂, wherein R⁴ is as defined herein, Y is substituted or unsubstituted cycloalkyl, and R⁶ is pyridylalkyl.

In another embodiment of the compounds of formula (II), L³ is CH₂ and L⁴ is CH₂, wherein R⁴ is as defined herein, Y is substituted or unsubstituted 5,6,7,8,-tetrahydroquinolin-8-yl, and R⁶ is H.

In another embodiment of the compounds of formula (II), L³ is CH₂ and L⁴ is CH₂, wherein R⁴ is as defined herein, Y is substituted or unsubstituted 5,6,7,8,-tetrahydroquinolin-8-yl, and R⁶ is alkyl.

In another embodiment of the compounds of formula (II), L³ is CH₂ and L⁴ is CH₂, wherein R⁴ is as defined herein, Y is substituted or unsubstituted 5,6,7,8,-tetrahydroquinolin-8-yl, and R⁶ is methyl.

In another embodiment of the compounds of formula (II), L³ is CH₂ and L⁴ is CH₂, wherein R⁴ is as defined herein, Y is substituted or unsubstituted 5,6,7,8,-tetrahydroquinolin-8-yl, and R⁶ is arylalkyl.

In another embodiment of the compounds of formula (II), L³ is CH₂ and L⁴ is CH₂, wherein R⁴ is as defined herein, Y is substituted or unsubstituted 5,6,7,8,-tetrahydroquinolin-8-yl, and R⁶ is heteroarylalkyl.

In another embodiment of the compounds of formula (II), L³ is CH₂ and L⁴ is CH₂, wherein R⁴ is as defined herein, Y is substituted or unsubstituted 5,6,7,8,-tetrahydroquinolin-8-yl, and R⁶ is imidazolylalkyl.

In another embodiment of the compounds of formula (II), L³ is CH₂ and L⁴ is CH₂, wherein R⁴ is as defined herein, Y is substituted or unsubstituted 5,6,7,8,-tetrahydroquinolin-8-yl, and R⁶ is pyridylalkyl.

### DEFINITIONS

The term “organism” refers to any living entity comprised of at least one cell. A living organism can be as simple as, for example, a single eukaryotic cell or as complex as a mammal, including a human being.

The term “chemokine receptor modulator” means a substance including but not limited to a molecule, polypeptide, polynucleotide, inhibitory polynucleotide, or siRNA, that interferes or inhibits the biological activity of the chemokine receptor including, but not limited to, the binding of a ligand to the receptor.

The term “chemokine antagonist” means a polypeptide that specifically binds to a chemokine receptor, particularly polypeptides that are not an antibody.

Representative chemokine peptide antagonists include T140 and derivatives of T140. Exemplary derivatives of T140 include, but are not limited to, TN14003, TC14012, and TE14011 as well as those found in Tamamura, H. et al. Synthesis of potent CXCR4 inhibitors possessing low cytotoxicity and improved biostability based on T140 derivatives, Org. Biomol. Chem. 1:3656-3662, 2003, which is incorporated by reference herein in its entirety.

The term “therapeutically effective amount” or “effective amount”, as used herein, means an amount of a compound or composition which is sufficient enough to significantly and positively modify the symptoms and/or conditions to be treated (e.g., provide a positive clinical response). The effective amount of an active ingredient for use in a pharmaceutical composition will vary with the particular condition being treated, the severity of the condition, the duration of the treatment, the nature of concurrent therapy, and the particular active ingredient(s) being employed, and like factors within the knowledge and expertise of the attending physician. For example, in reference to cancer or pathologies related to unregulated cell division, a therapeutically effective amount refers to that amount which has the effect of (1) reducing the size of a tumor, (2) inhibiting (that is, slowing to some extent, preferably stopping) aberrant cell division, for example cancer cell division, (3) preventing or reducing the metastasis of cancer cells, and/or, (4) relieving to some extent (or, preferably, eliminating) one or more symptoms associated with a pathology related to or caused in part by unregulated or aberrant cellular division, including for example, cancer, or angiogenesis.

A “pharmaceutical composition” refers to a mixture of one or more of the compounds described herein, or pharmaceutically acceptable salts thereof, with other chemical components, such as physiologically acceptable carriers and excipients. One purpose of a pharmaceutical composition is to facilitate administration of a compound to an organism.

As used herein, a “pharmaceutically acceptable carrier” refers to a carrier or diluent that does not cause significant irritation to an organism and does not abrogate the biological activity and proper-ties of the administered compound.

An “excipient” refers to an inert substance added to a pharmaceutical composition to further facilitate administration of a compound, such as binders, anti-adherents, coatings, disintegrants, fillers, diluents, flavors, colors, glidants, lubricants, preservatives, sorbitans, and sweeteners. Examples, without limitation, of excipients include calcium carbonate, calcium phosphate, various sugars and types of starch, cellulose derivatives, gelatin, vegetable oils and polyethylene glycols.

“Treating” or “treatment” of a disease includes preventing the disease from occurring in an animal that may be predisposed to the disease but does not yet experience or exhibit symptoms of the disease (prophylactic treatment), inhibiting the disease (slowing or arresting its development), providing relief from the symptoms or side-effects of the disease (including palliative treatment), and relieving the disease (causing regression of the disease). With regard to HIV or cancer, these terms simply mean that the life expectancy of an individual affected with HIV or cancer will be increased or that one or more of the symptoms of the disease will be reduced.

The term “prodrug” refers to an agent, including nucleic and polypeptides, which is converted into a biologically active form in vivo. Prodrugs are often useful because, in some situations, they may be easier to administer than the parent compound. They may, for instance, be bioavailable by oral administration whereas the parent compound is not. The prodrug may also have improved solubility in pharmaceutical compositions over the parent drug. A prodrug may be converted into the parent drug by various mechanisms, including enzymatic processes and metabolic hydrolysis. Harper, N.J. (1962). Drug Latentiation in Jucker, ed. Progress in Drug Research, 4:221-294; Morozowich et al. (1977). Application of Physical Organic Principles to Prodrug Design in E. B. Roche ed. Design of Biopharmaceutical Properties through Prodrugs and Analogs, APhA; Acad. Pharm. Sci.; E. B. Rocke, ed. (1977). Bioreversible Carriers in Drug in Drug Design, Theory and Application, APhA; H. Bundgaard, 15 ed. (1985) Design of Prodrugs, Elsevier; Wang et al. (1999) Prodrug approaches to the improved delivery of peptide drug, Curr. Pharm. Design. 5(4):265-287; Pauletti et al. (1997). Improvement in peptide bioavailability: Peptidomimetics and Prodrug Strategies, Adv. Drug. Delivery Rev. 27:235-256; Mizen et al. (1998). The Use of Esters as Prodrugs for Oral Delivery of P-Lactam antibiotics, Pharm. Biotech. 11,:345-365; Gaignault et al. (1996). Designing Prodrugs and Bioprecursors I. Carrier Prodrugs, Pract. Med. Chem. 671-696; M. Asgharnejad (2000). Improving Oral Drug Transport Via Prodrugs, in G. L. Amidon, P. I. Lee and E. M. Topp, Eds., Transport Processes in Pharmaceutical Systems, Marcell Dekker, p. 185-21 8; Balant et al. (1990) Prodrugs for the improvement of drug absorption via different routes of administration, Eur. J. Drug Metab. Pharmacokinet., 15(2): 143-53; Balimane and Sinko (1999). Involvement of multiple transporters in the oral absorption of nucleoside analogues, Adv. Drug Delivery Rev., 39(1-3): 1 83-209; Browne (1997). Fosphenyloin (Cerebyx), Clin. Neuropharmacol. 20(1): 1-1 2; Bundgaard (1979). Bioreversible derivatization of drugs—principle and applicability to improve the therapeutic effects of drugs, Arch. Pharm. Chemi. 86(1): 1-39; H. Bundgaard, ed. (1985) Design of Prodrugs, New York: Elsevier; Fleisher et al. (1 996). Improved oral drug delivery: solubility limitations overcome by the use of prodrugs, Adv. Drug Delivery Rev, 19(2): 1 15-130; Fleisher et al. (1985). Design of prodrugs for improved gastrointestinal absorption by intestinal enzyme targeting, Methods Enzymol. 112:360-81; Farquhar D, et al. (1983). Biologically Reversible Phosphate-Protective Groups, J. Pharm. Sci., 72(3): 24-325; Han, H. K. et al. (2000). Targeted prodrug design to optimize drug delivery, AAPS Pharm Sci., 2(1): E6; Sadzuka Y. (2000). Effective prodrug liposome and conversion to active metabolite, Curr. Drug Metab., 1(1):31-48; D. M. Lambert (2000) Rationale and applications of lipids as prodrug carriers, Eur. J. Pharm. Sci, 11 Suppl. 2:S15-27; Wang, W. et al. (1999) Prodrug approaches to the improved delivery of peptide drugs. Gurr. Pharm. Des., 5(4):265-87.

As used herein, the term “topically active agents” refers to compositions of the present disclosure that elicit pharmacological responses at the site of application (contact) to a host.

As used herein, the term “topically” refers to application of the compositions of the present disclosure to the surface of the skin and mucosal cells and tissues.

The term “nucleic acid” is a term of art that refers to a string of at least two base-sugar-phosphate combinations. For naked DNA delivery, a polynucleotide contains more than 120 monomeric units since it must be distinguished from an oligonucleotide. However, for purposes of delivering RNA, RNAi and siRNA, either single or double stranded, a polynucleotide contains 2 or more monomeric units. Nucleotides are the monomeric units of nucleic acid polymers. The term includes deoxyribonucleic acid (DNA) and ribonucleic acid (RNA) in the form of a messenger RNA, anti-sense, plasmid DNA, parts of a plasmid DNA or genetic material derived from a virus. Anti-sense is a polynucleotide that interferes with the function of DNA and/or RNA. Natural nucleic acids have a phosphate backbone, artificial nucleic acids may contain other types of backbones, but contain the same bases. RNA may be in the form of an tRNA (transfer RNA), snRNA (small nuclear RNA), rRNA (ribosomal RNA), mRNA (messenger RNA), anti-sense RNA, RNAi, siRNA, and ribozymes. The term also includes PNAs (peptide nucleic acids), phosphorothioates, and other variants of the phosphate backbone of native nucleic acids.

The term “siRNA” means a small inhibitory ribonucleic acid. The siRNA are typically less than 30 nucleotides in length and can be single or double stranded. The ribonucleotides can be natural or artificial and can be chemically modified. Longer siRNAs can comprise cleavage sites that can be enzymatically or chemically cleaved to produce siRNAs having lengths less than 30 nucleotides, typically 21 to 23 nucleotides. siRNAs share sequence homology with corresponding target mRNAs. The sequence homology can be 100 percent or less but sufficient to result is sequence specific association between the siRNA and the targeted mRNA.

The term “inhibitory nucleic acid” means an RNA, DNA, or combination thereof that interferes or interrupts the translation of mRNA. Inhibitory nucleic acids can be single or double stranded. The nucleotides of the inhibitory nucleic acid can be chemically modified, natural or artificial.

The term “prophylactically effective amount” refers to an amount effective, at dosages and for periods of time necessary, to achieve the desired prophylactic result, such as modulation of CXCR4, SDF-1 activity. A prophylactically effective amount can be determined as described herein for an effective amount. Typically, since a prophylactic dose is used in subjects prior to or at an earlier stage of disease, the prophylactically effective amount will be less than a therapeutically effective amount.

Abbreviations used include: CXCR4, CXC Chemokine receptor-4; SDF-1 stromal-derived factor-1; FACS, fluorescence-activated cell sorter; VEGF, vascular endothelial growth factor; MTT, methylthiazoletetrazolium; RT-PCR, Reverse transcription Polymerase Chain Reaction; MAb, monoclonal antibody; PE, R-Phycoerithrin; SCID, Severe Combined Immunodeficient; CC₅₀, 50% cytotoxic concentration; EC₅₀, 50% effective concentration; SI, selective index (CC₅₀/EC₅₀); DCIS, Ductal carcinoma in situ, H&E, hematoxylin and eosin; siRNA, small interfering RNA; HPRT, hypoxanthine-guanine-phosphoribosyltransferase.

The term “alkyl”, as used herein, unless otherwise specified, includes but is not limited to a saturated straight or branched, primary, secondary, or tertiary hydrocarbon of C₁ to C₂₀ or C₁ to C₁₀ and specifically includes methyl, ethyl, propyl, isopropyl, cyclopropyl, butyl, isobutyl, t-butyl, pentyl, cyclopentyl, isopentyl, neopentyl, hexyl, isohexyl, cyclohexyl, cyclohexylmethyl, 3-methylpentyl, 2,2-dimethylbutyl, and 2,3-dimethylbutyl. The term optionally includes substituted alkyl groups. Moieties with which the alkyl group can be substituted are selected from the group consisting of halo (e.g., trifluoromethyl), hydroxyl, amino, alkylamino, arylamino, alkoxy, aryloxy, nitro, cyano, sulfonic acid, sulfate, phosphonic acid, phosphate, or phosphonate, either unprotected, or protected as necessary, as known to those skilled in the art, for example, as taught in Greene, et al., Protective Groups in Organic Synthesis, John Wiley and Sons, Second Edition, 1991, hereby incorporated by reference.

Whenever the terms “C₁-C₅ alkyl”, “C₂-C₅ alkenyl”, “C₁-C₅ alkoxy”, “C₂-C₅ alkenoxy”, “C₂-C₅ alkynyl”, and “C₂-C₅ alkynoxy” are used, these are considered to include, independently, each member of the group, such that, for example, C₁-C₅ alkyl includes straight, branched and where appropriate cyclic C1, C2, C3, C4 and C5 alkyl functionalities; C₂-C₅ alkenyl includes straight, branched, and where appropriate cyclic C₂, C₃, C₄ and C₅ alkenyl functionalities; C₁-C₅ alkoxy includes straight, branched, and where appropriate cyclic C₁, C₂, C₃, C₄ and C₅ alkoxy functionalities; C₂-C₅ alkenoxy includes straight, branched, and where appropriate cyclic C₂, C₃, C₄ and C₅ alkenoxy functionalities; C₂-C₅ alkynyl includes straight, branched and where appropriate cyclic C₁, C₂, C₃, C₄ and C₅ alkynyl functionalities; and C₂-C₅ alkynoxy includes straight, branched, and where appropriate cyclic C₂, C₃, C₄ and C₅ alkynoxy functionalities.

The term “lower alkyl”, as used herein, and unless otherwise specified, includes a C₁ to C₄ saturated straight or branched alkyl group, optionally including substituted forms. Unless otherwise specifically stated in this application, when alkyl is a suitable moiety, lower alkyl is preferred. Similarly, when alkyl or lower alkyl is a suitable moiety, unsubstituted alkyl or lower alkyl is preferred.

The term “amino” includes an amine group (i.e., —NH₂) as well as an amine group substituted with one or more alkyl groups (as defined herein), substituted alkyl groups (e.g., hydroxyalkyl, alkoxyalkyl, thioalkyl, alkylthioalkyl, etc.), one or two aryl groups (as defined herein), one or two heteroaryl groups (as defined herein), one or two arylalkyl groups (as defined herein), one or two heteroarylalkyl groups (as defined herein), combinations of H, alkyl, aryl, heteroaryl, arylalkyl, and heteroarylalkyl groups. When the amino group has one or more alkyl, aryl, heteroaryl, arylalkyl, or heteroarylalkyl groups, the alkyl, aryl, heteroaryl, arylalkyl, or heteroarylalkyl groups can be unsubstituted or substituted. The terms “alkylamino” or “arylamino” refer to an amino group that has one or two alkyl or aryl substituents, respectively. The terms “arylalkylamino” or “heteroarylalkylamino” refer to an amino group that has one or two arylalkyl or heteroaryl alkyl groups, respectively.

The term “amino” can also include amino groups substituted with one or more acyl groups such as —C(O)-alkyl, —C(O)-aryl, —C(O)-heteroaryl, —C(O)—O-alkyl, —C(O)—O-aryl, —C(O)—O-heteroaryl, —C(O)—N(R)-alkyl, —C(O)—N(R)-aryl, —C(O)—N(R)— heteroaryl; (—C(O)OR)₂, sulfonyl groups such as —S(O)₂-alkyl, —S(O)₂-aryl, —S(O)₂-heteroaryl, —S(O)₂—O-alkyl, —S(O)₂—O-aryl, —S(O)₂—O-heteroaryl, —S(O)₂—NH₂, —S(O)₂—N(R)-alkyl, —S(O)₂—N(R)-aryl, —S(O)₂—N(R)-heteroaryl, etc. (wherein R¹ is H, alkyl, aryl, heteroaryl). When the substituent on the amino group is an acyl group, the moiety can also be referred to as an “amido” group (i.e., when the acyl group is —C(O)-alkyl, —C(O)-aryl, or —C(O)-heteroaryl), a “urea” moiety (i.e., when the acyl group is —C(O)—N(R)-alkyl, —C(O)—N(R)-aryl, or —C(O)—N(R)-heteroaryl), or a “urethane” moiety (i.e., when the acyl group is —C(O)—O-alkyl, —C(O)—O-aryl, —C(O)—O-arylalkyl, or —C(O)—O-heteroaryl). When the substituents on the amino group are two acyl groups, the moiety can also be referred to as an “imido” group (i.e., when the two acyl groups are (—C(O)-alkyl)₂, (—C(O)-aryl)₂, or (C(O)-heteroaryl)₂). An example of an imido group is phthalimido. Unless stated to the contrary, a substituent is bound to a structure through the last named moiety of the substituent. For example, an “arylalkyl” substituent is bound to a structure through the “alkyl” moiety of the substituent.

The term “aminoalkyl”, as defined herein means an amino group bonded to the parent moiety through an alkyl moiety (i.e., amino-alkyl-), wherein the amino and alkyl portions of the aminoalkyl are each as defined herein. Non-limiting examples of amino alkyl include (CH₃)₂N—CH₂CH₂—, CH₃—O—CH₂CH₂NH—CH₂—, etc.

The term “amido” or “aminocarbonyl” means a —C(O)-amine group, wherein the “amine” moiety is as defined herein. Non-limiting examples of aminoacyl include phenyl-NH—C(O)—, piperazine-C(O)—, (CH₃—O—CH₂CH₂)₂N—C(O)—, pyridine-CH₂—NH—C(O)—, phenyl-CH₂—NH—C(O)—, etc.

The term “protected” as used herein and unless otherwise defined refers to a group that is added to an oxygen, nitrogen, or phosphorus atom to prevent its further reaction or for other purposes. A wide variety of oxygen and nitrogen protecting groups are known to those skilled in the art of organic synthesis.

The term “aryl”, as used herein, means an aromatic monocyclic or multicyclic ring system comprising about 6 to about 14 carbon atoms, preferably about 6 to about 10 carbon atoms. Non-limiting examples of suitable aryl groups include phenyl, biphenyl, or naphthyl. The term aryl refers to unsubstituted aryl groups or aryl groups substituted with one or more substituents which may be the same or different. The aryl group can be substituted with one or more substituents, including but not limited to substituents selected from the group consisting of hydroxyl, thiol, amino, alkylamino, arylamino, alkoxy, aryloxy, nitro, cyano, sulfonic acid, halo (F, Cl, I, Br), carboxy, ester, acyl, alkyl (i.e., any of the alkyl groups described herein, such as methyl, ethyl, propyl, butyl, etc.), alkenyl (i.e., any of the alkenyl groups described herein, such as vinyl, allyl, 1-propenyl, 1-butenyl, 2-butenyl, etc.), alkynyl (i.e., any of the alkynyl groups described herein, such as 1-ethynyl, 1-propynyl, 2-propynyl, etc.), haloalkyl (i.e., any of the haloalkyl groups described herein), sulfate, sulfonate, sulfonic esters and amides, phosphoric acid, phosphonic acid, phosphate, or phosphonate, either unprotected, or protected as necessary, as known to those skilled in the art, for example, as taught in Greene, et al., Protective Groups in Organic Synthesis, John Wiley and Sons, Second Edition, 1991.

The term “alkaryl” or “alkylaryl” refers to an alkyl group with an aryl substituent. In one embodiment, the “alk” or “alkyl” portion of the alkaryl is a lower alkyl group. Non-limiting examples of suitable alkylaryl groups include o-tolyl, p-tolyl and xylyl. The bond to the parent moiety is through the aryl.

The term “aralkyl” or “arylalkyl” refers to an aryl group attached to an alkyl group. In one embodiment, the “alk” or “alkyl” portion of the aralkyl is a lower alkyl group. Non-limiting examples of suitable aralkyl groups include benzyl, 2-phenethyl and naphthalenylmethyl. The bond to the parent moiety is through the alkyl. The aryl portion of the arylalkyl group may be substituted or unsubstituted.

The term “alkoxy”, as used herein, means alkyl-O—, wherein the alkyl moiety of the alkoxy group is an alkyl group as defined herein.

The term “cycloalkyl” means a non-aromatic mono- or multicyclic fused ring system comprising 3 to 10 ring carbon atoms, preferably 3 to 7 ring carbon atoms, more preferably 3 to 6 ring carbon atoms. The cycloalkyl can be optionally substituted with one or more substituents which may be the same or different. Non-limiting examples of suitable monocyclic cycloalkyls include cyclopropyl, cyclobutyl, cyclopentyl, cyclohexyl and the like. Non-limiting examples of suitable multicyclic cycloalkyls include 1-decalinyl, norbornenyl, adamantyl and the like. Suitable substituents for cycloalkyls include substituents selected from the group consisting of hydroxyl, thiol, amino, alkylamino, arylamino, alkoxy, aryloxy, nitro, cyano, sulfonic acid, halo (F, Cl, I, Br), carboxy, ester, acyl, alkyl (i.e., any of the alkyl groups described herein, such as methyl, ethyl, propyl, butyl, etc.), alkenyl (i.e., any of the alkenyl groups described here rein, such as vinyl, allyl, 1-propenyl, 1-butenyl, 2-butenyl, etc.), alkynyl (i.e., any of the alkynyl groups described herein, such as 1-ethynyl, 1-propynyl, 2-propynyl, etc.), haloalkyl (i.e., any of the haloalkyl groups described herein), sulfate, sulfonate, sulfonic esters and amides, phosphoric acid, phosphonic acid, phosphate, or phosphonate, either unprotected, or protected as necessary, as known to those skilled in the art, for example, as taught in Greene, et al., Protective Groups in Organic Synthesis, John Wiley and Sons, Second Edition, 1991, herein incorporated by reference in its entirety. Substituents can also include fused aromatic rings, e.g.:

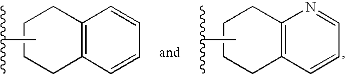

wherein the fused aromatic or heteroaromatic ring can itself be unsubstituted or substituted with one or more substituents as described herein.

The term “halo”, as used herein, includes chloro, bromo, iodo, and fluoro.

The term “haloalkyl”, as used herein, means an alkyl as defined above wherein one or more hydrogen atoms on the alkyl are replaced by a halo defined above. Non-limiting examples of haloalkyl groups include —CF₃, —CHF₂, —CH₂F, —CH₂CF₃, etc.

The term “hydroxyalkyl”, as used herein, means an alkyl group having at least one hydroxy substituent. Non-limiting examples of hydroxyalkyl groups include hydroxyethyl, 3-hydroxypropyl, 2-hydroxy propyl, etc.

The term “alkoxyalkyl”, as used herein, means alkyl-O-alkyl-, wherein each of the alkyl moieties is as defined herein. The skilled practitioner will recognize that a divalent alkyl group (i.e., an alkyl group bonded to two other moieties) can also be referred to as an “alkylene” group. An alkylene group is an alkyl group in which one of the C—H bonds is replaced with a covalent bond to another moiety. Non-limiting examples of alkoxyalkyl groups include CH₃—O—CH₂CH₂—, CH₃—O—CH₂CH₂CH₂—, CH₃CH₂—O—CH₂CH₂—, CH₃CH₂—O—CH₂CH₂CH₂—, t-Bu-O—CH₂CH₂—, etc.

The term “acyl” refers to a carboxylic acid ester in which the non-carbonyl moiety of the ester group is selected from straight, branched, or cyclic alkyl or lower alkyl, alkoxyalkyl including methoxymethyl, aralkyl including benzyl, aryloxyalkyl such as phenoxymethyl, aryl including phenyl optionally substituted with halogen, C₁ to C₄ alkyl or C₁ to C₄ alkoxy, sulfonate esters such as alkyl or aralkyl sulphonyl including methanesulfonyl, the mono, di- or triphosphate ester, trityl or monomethoxytrityl, substituted benzyl, trialkylsilyl (e.g. dimethyl-t-butylsilyl) or diphenylmethylsilyl. In one embodiment, aryl groups in the esters comprise a phenyl group. The term “lower acyl” refers to an acyl group in which the non-carbonyl moiety is a lower alkyl.

The term “carboxy”, as used herein, means —C(O)OH.

The term “alkoxycarbonyl”, as used herein, means —C(O)—O-alkyl, wherein the alkyl moiety is any alkyl as defined herein.

The term “aminocarbonyl”, as used herein, means —C(O)-amino, wherein the amino moiety is any amino as defined herein.

The term “alkylthioalkyl”, as used herein, means alkyl-5-alkyl-, wherein each of the alkyl moieties is as defined herein. Non-limiting examples of alkylthioalkyl groups include CH₃—S—CH₂CH₂—, CH₃—S—CH₂CH₂CH₂—, CH₃CH₂—S—CH₂CH₂—, CH₃CH₂—S—CH₂CH₂CH₂—, t-Bu-S—CH₂CH₂—, etc.

With reference to the number of moieties (e.g., substituents, groups or rings) in a compound, unless otherwise defined, the phrases “one or more” and “at least one” mean that there can be as many moieties as chemically permitted, and the determination of the maximum number of such moieties is well within the knowledge of those skilled in the art.

The term “pharmaceutically acceptable salt, solvate, ester or prodrug” is used throughout the specification to describe any pharmaceutically acceptable form (such as an ester, phosphate ester, salt of an ester or a related group, or hydrate) of a compound which, upon administration to a patient, provides the compound described in the specification. Pharmaceutically acceptable salts include those derived from pharmaceutically acceptable inorganic or organic bases and acids such as hydrochloric acid, hydrobromic acid, sulfuric acid, nitric acid, phosphoric acid, methanesulfonic acid, ethanesulfonic acid, p-toluensulfonic acid, salicylic acid, malic acid, maleic acid, succinic acid, tartaric acid, citric acid and the like. Suitable salts include those derived from alkali metals such as potassium and sodium, alkaline earth metals such as calcium and magnesium, among numerous other acids well known in the art, for example as described herein.

Prodrugs and solvates of the compounds of the invention are also contemplated herein. The term “prodrug”, as employed herein, denotes a compound that is a drug precursor (e.g., has one or more biologically labile protecting group(s) on a functional moiety of the active compound) which, upon administration to a subject, undergoes chemical conversion by metabolic or chemical processes (e.g., oxidation, reduction, amidation, deamination, hydroxylation, dehydroxylation, hydrolysis, dehydrolysis, alkylation, dealkylation, acylation, deacylation, phosphorylation, dephosphorylation, etc.) to yield an active compound or a salt and/or solvate thereof. A discussion of prodrugs is provided in T. Higuchi and V. Stella, Pro-drugs as Novel Delivery Systems (1987) Volume 14 of the A.C.S. Symposium Series, and in Bioreversible Carriers in Drug Design, (1987) Edward B. Roche, ed., American Pharmaceutical Association and Pergamon Press, both of which are incorporated herein by reference thereto.

The term “solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O.

The term “heterocyclic” or “heterocyclyl” refers to a cyclic group that may be unsaturated, partially or fully saturated and wherein there is at least one heteroatom, such as oxygen, sulfur, nitrogen, or phosphorus in the ring. Heterocyclic or heterocyclyl groups include heteroaryl groups. Non-limiting examples of non-aromatic heterocyclyls include piperidinyl, piperazinyl, morpholinyl, pyrrolidinyl, morpholino, thiomorpholino, oxiranyl, pyrazolinyl, dioxolanyl, 1,4-dioxanyl, aziridinyl, tetrahydrofuranyl, pyrrolinyl, dihydrofuranyl, dioxanyl, tetrahydropyranyl, dihydropyranyl, indolinyl, imidazolyl, tetraazacyclotetradecanyl, dioxadiazacyclododecanyl, diazepanyl, etc., wherein each of the aforementioned heterocyclyls can be unsubstituted or substituted with one or more substituents.

The term “heteroaryl” or “heteroaromatic”, as used herein, refers to an aromatic heterocyclyl ring that includes at least one sulfur, oxygen, nitrogen or phosphorus in the aromatic ring. Nonlimiting examples of heteroaromatics are furanyl, pyridyl, pyrimidinyl, benzoxazolyl, 1,2,4-oxadiazolyl, 1,3,4-oxadiazolyl, 1,3,4-thiadiazole, indazolyl, 1,3,5-triazinyl, thienyl, tetrazolyl, benzofuranyl, quinolyl, isoquinolyl, benzothienyl, isobenzofuryl, indolyl, isoindolyl, benzimidazolyl, purine, carbazolyl, oxazolyl, thiazolyl, benzothiazolyl, isothiazolyl, 1,2,4-thiadiazolyl, isooxazolyl, pyrrolyl, quinazolinyl, cinnolinyl, phthalazinyl, xanthinyl, hypoxanthinyl, pyrazole, 1,2,3-triazole, 1,2,4-triazole, 1,2,3-oxadiazole, thiazine, pyridazine, benzothiophenyl, isopyrrole, thiophene, pyrazine, or pteridinyl wherein said heteroaryl or heterocyclic group can be optionally substituted with one or more substituents. In one embodiment, heterocyclyl and heteraromatic groups include purine and pyrimidines.

Substituted aromatic or heteroaromatic rings (including aromatic or heteroaromatic portions of functional groups such as arylalkyl or heteroarylalkyl groups) can be substituted with one or more substituents. Non-limiting examples of such substituents selected from the group consisting of hydroxyl, thiol, amino, alkylamino, arylamino, alkoxy, aryloxy, nitro, cyano, sulfonic acid, halo (F, Cl, I, Br), carboxy, ester, acyl, alkyl (i.e., any of the alkyl groups described herein, such as methyl, ethyl, propyl, butyl, etc.), alkenyl (i.e., any of the alkenyl groups described here rein, such as vinyl, allyl, 1-propenyl, 1-butenyl, 2-butenyl, etc.), alkynyl (i.e., any of the alkynyl groups described herein, such as 1-ethynyl, 1-propynyl, 2-propynyl, etc.), haloalkyl (i.e., any of the haloalkyl groups described herein), sulfate, sulfonate, sulfonic esters and amides, phosphoric acid, phosphonic acid, phosphate, or phosphonate, either unprotected, or protected as necessary, as known to those skilled in the art, for example, as taught in Greene, et al, Protective Groups in Organic Synthesis, John Wiley and Sons, Second Edition, 1991.

Functional oxygen and nitrogen groups (e.g., on a aryl or heteroaryl group) can be protected as necessary or desired. Suitable protecting groups are well known to those skilled in the art, and include trimethylsilyl, dimethylhexylsilyl, t-butyldimethylsilyl, and t-butyldiphenylsilyl, trityl or substituted trityl, alkyl groups, acycl groups such as acetyl and propionyl, methanesulfonyl, and p-toluenelsulfonyl.

The term purine or pyrimidine includes, but is not limited to, adenine, N₆-alkylpurines, N₆-acylpurines (wherein acyl is C(O)(alkyl, aryl, alkylaryl, or arylalkyl), N₆-benzylpurine, N₆-halopurine, N₆-vinylpurine, N₆-acetylenic purine, N₆-acyl purine, N₆-hydroxyalkyl purine, N₆-thioalkyl purine, N₂-alkylpurines, N₂-alkyl-6-thiopurines, thymine, cytosine, 5-fluorocytosine, 5-methylcytosine, 6-azapyrimidine, including 6-azacytosine, 2- and/or 4-mercaptopyrmidine, uracil, 5-halouracil, including 5-fluorouracil, C₅-alkylpyrimidines, C₅-benzylpyrimidines, C₅-halopyrimidines, C₅-vinylpyrimidine, C₅-acetylenic pyrimidine, C₅-acyl pyrimidine, C₅-hydroxyalkyl purine, C₅-amidopyrimidine, C₅-cyanopyrimidine, C₅-nitropyrimidine, C₅-aminopyrimidine, N₂-alkylpurines, N₂-alkyl-6-thiopurines, 5-azacytidinyl, 5-azauracilyl, triazolopyridinyl, imidazolopyridinyl, pyrrolopyrimidinyl, and pyrazolopyrimidinyl. Purine bases include, but are not limited to, guanine, adenine, hypoxanthine, 2,6-diaminopurine, and 6-chloropurine.

Compounds of the present invention, and salts, solvates and prodrugs thereof, may exist in their tautomeric form (for example, as an amide or imino ether). All such tautomeric forms are contemplated herein as part of the present invention. For example, the following structure:

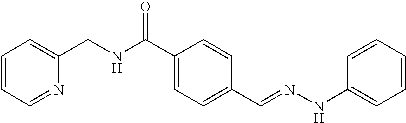

can also be represented as:

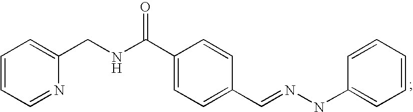

the following structure:

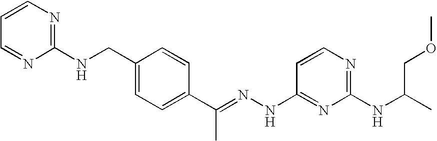

can also be represented as:

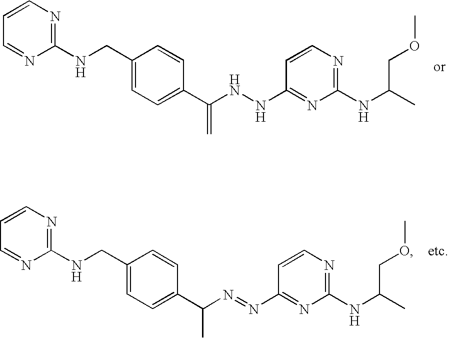

All stereoisomers (for example, geometric isomers, optical isomers and the like) of the present compounds (including those of the salts, solvates and prodrugs of the compounds as well as the salts and solvates of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this invention. Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations. The use of the terms “salt”, “solvate” “prodrug” and the like, is intended to equally apply to the salt, solvate and prodrug of enantiomers, stereoisomers, rotamers, tautomers, racemates or prodrugs of the inventive compounds.

Polymorphic forms of the compounds of the present invention, and of the salts, solvates and/or prodrugs of the compounds of the present invention, are intended to be included in the present invention.

The compounds of the present are those compounds of formula (I), or pharmaceutically acceptable salts, solvates, prodrugs, tautomers, stereoisomers, and esters thereof, having sufficient chemical stability for formulation in a pharmaceutical composition. It should also be noted that any carbon or heteroatom with unsatisfied valences in the text, schemes, examples and Tables herein is assumed to have the hydrogen atom(s) to satisfy the valences.

Formulations

In cases where compounds are sufficiently basic or acidic to form stable nontoxic acid or base salts, administration of the compound as a pharmaceutically acceptable salt may be appropriate. Examples of pharmaceutically acceptable salts are organic acid addition salts formed with acids, which form a physiological acceptable anion, for example, tosylate, methanesulfonate, acetate, citrate, malonate, tartarate, succinate, benzoate, ascorbate, .-ketoglutarate, and .-glycerophosphate. Suitable inorganic salts may also be formed, including, sulfate, nitrate, bicarbonate, and carbonate salts.

Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

Exemplary acid addition salts include acetates, adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides, hydrobromides, hydroiodides, 2-hydroxyethanesulfonates, lactates, maleates, methanesulfonates, 2-naphthalenesulfonates, nicotinates, nitrates, oxalates, pectinates, persulfates, 3-phenylpropionates, phosphates, picrates, pivalates, propionates, salicylates, succinates, sulfates, sulfonates (such as those mentioned herein), tartarates, thiocyanates, toluenesulfonates (also known as tosylates,) undecanoates, and the like. Additionally, acids which are generally considered suitable for the formation of pharmaceutically useful salts from basic pharmaceutical compounds are discussed, for example, by S. Berge et al, Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

Exemplary basic salts include ammonium salts, alkali metal salts such as sodium, lithium, and potassium salts, alkaline earth metal salts such as calcium and magnesium salts, salts with organic bases (for example, organic amines) such as benzathines, dicyclohexylamines, hydrabamines (formed with N,N-bis(dehydroabietyl)ethylenediamine), N-methyl-D-glucamines, N-methyl-D-glucamides, t-butyl amines, and salts with amino acids such as arginine, lysine and the like. Basic nitrogen-containing groups may be quarternized with agents such as lower alkyl halides (e.g. methyl, ethyl, propyl, and butyl chlorides, bromides and iodides), dialkyl sulfates (e.g. dimethyl, diethyl, dibutyl, and diamyl sulfates), long chain halides (e.g. decyl, lauryl, myristyl and stearyl chlorides, bromides and iodides), aralkyl halides (e.g. benzyl and phenethyl bromides), and others.

All such acid salts and base salts are intended to be pharmaceutically acceptable salts within the scope of the invention and all acid and base salts are considered equivalent to the free forms of the corresponding compounds for purposes of the invention.

The active compound can also be provided as a prodrug, which is converted into a biologically active form in vivo. A prodrug may be converted into the parent drug by various mechanisms, including enzymatic processes and metabolic hydrolysis: T. Higuchi and V. Stella, Pro-drugs as Novel Delivery Systems, Vol. 14 of A.C.S. Symposium Series (1987)Harper, N.J. (1962) in Jucker, ed. Progress in Drug Research, 4:221-294; Morozowich et al. (1977) in E. B. Roche ed. Design of Biopharmaceutical Properties through Prodrugs and Analogs, APhA (Acad. Pharm. Sci.); E. B. Roche, ed. (1977) Bioreversible Carriers in Drug in Drug Design, Theory and Application, APhA; H. Bundgaard, ed. (1985) Design of Prodrugs, Elsevier; Wang et al. (1999) Curr. Pharm. Design. 5(4):265-287; Pauletti et al. (1997) Adv. Drug. Delivery Rev. 27:235-256; Mizen et al. (1998) Pharm. Biotech. 11:345-365; Gaignault et al. (1996) Pract. Med. Chem. 671-696; M. Asghamejad (2000) in G. L. Amidon, P. I. Lee and E. M. Topp, Eds., Transport Proc. Pharm. Sys., Marcell Dekker, p. 185-218; Balant et al. (1990) Eur. J. Drug Metab. Pharmacokinet., 15(2): 143-53; Balimane and Sinko (1999) Adv. Drug Deliv. Rev., 39(1-3):183-209; Browne (1997). Clin. Neuropharm. 20(1): 1-12; Bundgaard (1979) Arch. Pharm. Chemi. 86(1): 1-39; H. Bundgaard, ed. (1985) Design of Prodrugs, New York: Elsevier; Fleisher et al. (1996) Adv. Drug Delivery Rev, 19(2): 115-130; Fleisher et al. (1985) Methods Enzymol. 112: 360-81; Farquhar D, et al. (1983) J. Pharm. Sci., 72(3): 324-325; Han, H. K. et al. (2000) AAPS Pharm Sci., 2(1): E6; Sadzuka Y. (2000) Curr. Drug Metab., 1:31-48; D. M. Lambert (2000) Eur. J. Pharm. Sci., 11 Suppl 2:S1 5-27; Wang, W. et al. (1999) Curr. Pharm. Des., 5(4):265, each of which is incorporated herein by reference in its entirety.

The active compound can also be provided as a lipid prodrug. Nonlimiting examples of U.S. patents that disclose suitable lipophilic substituents that can be covalently incorporated into the compound or in lipophilic preparations, include U.S. Pat. Nos. 5,149,794 (Sep. 22, 1992, Yatvin et al.); 5,194,654 (Mar. 16, 1993, Hostetler et al., 5,223,263 (Jun. 29, 1993, Hostetler et al.); 5,256,641 (Oct. 26, 1993, Yatvin et al.); 5,411,947 (May 2, 1995, Hostetler et al.); 5,463,092 (Oct. 31, 1995, Hostetler et al.); 5,543,389 (Aug. 6, 1996, Yatvin et al.); 5,543,390 (Aug. 6, 1996, Yatvin et al.); 5,543,391 (Aug. 6, 1996, Yatvin et al.); and 5,554,728 (Sep. 10, 1996; Basava et al.).

Preferably, the pharmaceutical preparation is in a unit dosage form. In such form, the preparation is subdivided into suitably sized unit doses containing appropriate quantities of the active compound, e.g., an effective amount to achieve the desired purpose.

The actual dosage employed may be varied depending upon the requirements of the patient and the severity of the condition being treated. Determination of the proper dosage regimen for a particular situation is within the skill of the art. For convenience, the total daily dosage may be divided and administered in portions during the day as required.

For preparing pharmaceutical compositions from the compounds described by this invention, inert, pharmaceutically acceptable carriers can be either solid or liquid. Solid form preparations include powders, tablets, dispersible granules, capsules, cachets and suppositories. The powders and tablets may be comprised of from about 0.1 to about 95 percent active compound. Suitable solid carriers are known in the art, e.g. magnesium carbonate, magnesium stearate, talc, sugar or lactose. Tablets, powders, cachets and capsules can be used as solid dosage forms suitable for oral administration. Examples of pharmaceutically acceptable carriers and methods of manufacture for various compositions may be found in A. Gennaro (ed.), Remington's Pharmaceutical Sciences, 18th Edition, (1990), Mack Publishing Co., Easton, Pa.

Liquid form preparations include solutions, suspensions and emulsions. As an example may be mentioned water or water-propylene glycol solutions for parenteral injection or addition of sweeteners and opacifiers for oral solutions, suspensions and emulsions. Liquid form preparations may also include solutions for intranasal administration.

Aerosol preparations suitable for inhalation may include solutions and solids in powder form, which may be in combination with a pharmaceutically acceptable carrier, such as an inert compressed gas, e.g. nitrogen.

Also included are solid form preparations which are intended to be converted, shortly before use, to liquid form preparations for either oral or parenteral administration. Such liquid forms include solutions, suspensions and emulsions.

The compounds of the invention may also be deliverable transdermally. The transdermal compositions can take the form of creams, lotions, aerosols and/or emulsions and can be included in a transdermal patch of the matrix or reservoir type as are conventional in the art for this purpose.

Method of Treatment

The compounds described herein are particularly useful for the treatment or prevention of a disorder associated with chemokine receptor binding or activation, and particularly for the treatment of HIV or AIDS in a host in need thereof.

In one embodiment, a method of treating or preventing HIV infection or reduction of symptoms associated with AIDS is provided including administering a compound of at least one of Formula (IA)-(1H) to a host. In certain embodiments, the compound can be provided to a host before treatment of infection with another compound. In a separate embodiment, the compound is provided to a patient that has been treated for HIV infection to reduce the likelihood of recurrence, or reduce mortality associated with AIDS related symptoms. In another embodiment, the compound is administered to a host at high risk of suffering from HIV infections.

Hosts, including humans suffering from, or at risk for, HIV infection can be treated by administering an effective amount of the active compound or a pharmaceutically acceptable prodrug or salt thereof in the presence of a pharmaceutically acceptable carrier or diluent.

The administration can be prophylactically for the prevention of HIV infection or reduction of symptoms associated with AIDS. The active materials can be administered by any appropriate route, for example, orally, parenterally, intravenously, intradermally, subcutaneously, or topically, in liquid or solid form. However, the compounds are particularly suited to oral delivery.

An exemplary dose of the compound will be in the range from about 1 to 50 mg/kg, preferably 1 to 20 mg/kg, of body weight per day, more generally 0.1 to about 100 mg per kilogram body weight of the recipient per day. The effective dosage range of the pharmaceutically acceptable salts and prodrugs can be calculated based on the weight of the parent compound to be delivered. If the salt, ester or prodrug exhibits activity in itself, the effective dosage can be estimated as above using the weight of the salt, ester, solvate, or prodrug, or by other means known to those skilled in the art.

The amount and frequency of administration of the compounds of the invention and/or the pharmaceutically acceptable salts thereof will be regulated according to the judgment of the attending clinician considering such factors as age, condition and size of the patient as well as the condition and/or severity of the symptoms being treated. A typical recommended daily dosage regimen for oral administration can range from about 0.1 mg/day to about 2000 mg/day, in one to four divided doses. In one embodiment, the compounds of the present invention are administered orally. In other embodiments, the compounds of the present invention can be administered through intravenous injection, subcutaneous injection, intramuscular injection, inhalation, transdermal absorption or intrarectal suppository.

In a separate embodiment, a method for the treatment or prevention of HIV infection or reduction of symptoms associated with AIDS by administering a compound of the present invention, or a pharmaceutically acceptable salt, solvate, prodrug, or ester thereof to a host in need of treatment is provided. The compounds of the invention, or a pharmaceutically acceptable salt, solvate, prodrug, or ester thereof can be administered to a host in need thereof to reduce the severity of AIDS related disorders. In one embodiment of the invention, the host is a human.

In another embodiment, the invention provides a method of treating symptoms associated with other infections associated with chemokine receptor activation, for example, liver diseases associated with flavivirus or pestivirus infection, and in particular, HCV or HBV, by contacting a cell with a compound of the present invention, or a pharmaceutically acceptable salt, solvate, prodrug, or ester thereof. The cell can be in a host animal, in particular in a human.

The compounds can treat or prevent HIV infection, or reduce the severity of AIDS related symptoms and diseases in any host. However, typically the host is a mammal and more typically is a human. In certain embodiments the host has been diagnosed with AIDS prior to administration of the compound, however in other embodiments, the host is merely infected with HIV and asymptomatic.

Generally, the disclosure provides compositions and methods for treating or preventing a chemokine mediated pathology by administering a compound of the present invention, or a pharmaceutically acceptable salt, solvate, prodrug, or ester thereof to a host in a therapeutic amount, for example in an amount sufficient to inhibit chemokine receptor signal transduction in a cell expressing a chemokine receptor or homologue thereof.

Another embodiment provides uses of a compound of the present invention, or a pharmaceutically acceptable salt, solvate, prodrug, or ester thereof for the treatment of, or for the manufacture of a medicament for the treatment of chemokine mediated pathologies including, but not limited to cancer. Still another embodiment provides uses of a chemokine peptide antagonist for the manufacture of medicament for the prevention of tumor cell metastasis in a mammal.

The compounds, or pharmaceutically acceptable salts, solvates, prodrugs, or esters thereof of the present invention described herein can be used to treat or prevent cancer, in particular the spread of cancer within an organism. Cancer is a general term for diseases in which abnormal cells divide without control. Cancer cells can invade nearby tissues and can spread through the bloodstream and lymphatic system to other parks of the body. It has been discovered that the administration of a chemokine receptor modulator to a host, for example a mammal, inhibits or reduces the metastasis of tumor cells, in particular breast cancer and prostate cancer.

There are several main types of cancer, and the disclosed compounds or compositions can be used to treat any type of cancer. For example, carcinoma is cancer that begins in the skin or in tissues that line or cover internal organs. Sarcoma is cancer that begins in bone, cartilage, fat, muscle, blood vessels, or other connective or supportive tissue.

Leukemia is cancer that starts in blood-forming tissue such as the bone marrow, and causes large numbers of abnormal blood cells to be produced and enter the bloodstream. Lymphoma is cancer that begins in the cells of the immune system.

When normal cells lose their ability to behave as a specified, controlled and coordinated unit, a tumor is formed. A solid tumor is an abnormal mass of tissue that usually does not contain cysts or liquid areas. A single tumor may even have different populations of cells within it with differing processes that have gone awry. Solid tumors may be benign (not cancerous), or malignant (cancerous). Different types of solid tumors are named for the type of cells that form them. Examples of solid tumors are sarcomas, carcinomas, and lymphomas. Leukemias (cancers of the blood) generally do not form solid tumors. The compositions described herein can be used to reduce, inhibit, or diminish the proliferation of tumor cells, and thereby assist in reducing the size of a tumor.

Representative cancers that may treated with the disclosed compositions and methods include, but are not limited to, bladder cancer, breast cancer, colorectal cancer, endometrial cancer, head & neck cancer, leukemia, lung cancer, lymphoma, melanoma, non-small-cell lung cancer, ovarian cancer, prostate cancer, testicular cancer, uterine cancer, cervical cancer, thyroid cancer, gastric cancer, brain stem glioma, cerebellar astrocytoma, cerebral astrocytoma, ependymoma, Ewing's sarcoma family of tumors, germ cell tumor, extracranial cancer, Hodgkin's disease, leukemia, acute lymphoblastic leukemia, acute myeloid leukemia, liver cancer, medulloblastoma, neuroblastoma, brain tumors generally, non-Hodgkin's lymphoma, ostessarcoma, malignant fibrous histiocytoma of bone, retinoblastoma, rhabdomyosarcoma, soft tissue sarcomas generally, supratentorial primitive neuroectodermal and pineal tumors, visual pathway and hypothalamic glioma, Wilms' tumor, acute lymphocytic leukemia, adult acute myeloid leukemia, adult non-Hodgkin's lymphoma, chronic lymphocytic leukemia, chronic myeloid leukemia, esophageal cancer, hairy cell leukemia, kidney cancer, multiple myeloma, oral cancer, pancreatic cancer, primary central nervous system lymphoma, skin cancer, small-cell lung cancer, among others.

A tumor can be classified as malignant or benign. In both cases, there is an abnormal aggregation and proliferation of cells. In the case of a malignant tumor, these cells behave more aggressively, acquiring properties of increased invasiveness.

Ultimately, the tumor cells may even gain the ability to break away from the microscopic environment in which they originated, spread to another area of the body (with a very different environment, not normally conducive to their growth) and continue their rapid growth and division in this new location. This as called metastasis. Once malignant cells have metastasized, achieving cure is more difficult.

Benign tumors have less of a tendency to invade and are less likely to metastasize. They do divide in an uncontrolled manner, though. Depending on their location, they can be just as life threatening as malignant lesions. An example of this would be a benign tumor in the brain, which can grow and occupy space within the skull, leading to increased pressure on the brain. The compositions provided herein can be used to treat benign or malignant tumors.

Pharmaceutical Compositions

In one embodiment, pharmaceutical compositions including at least one compound of the present invention, or a pharmaceutically acceptable salt, solvate, prodrug, or ester thereof is provided. In certain embodiments, at least a second active compound is administered in combination or alternation with the first compound.

The second active compound can be an antiviral, particularly an agent active against HIV and in a particular embodiment, active against HIV-1. Hosts, including humans suffering from or at risk of contracting HIV can be treated by administering an effective amount of a pharmaceutical composition of the active compound.

In another embodiment, the second active compound can be a chemotherapeutic agent, for example an agent active against a primary tumor. Hosts, including humans suffering from or at risk for a proliferative disorder can be treated by administering an effective amount of a pharmaceutical composition of the active compound.

The compound of the present invention, or a pharmaceutically acceptable salt, solvate, prodrug, or ester thereof is conveniently administered in unit any suitable dosage form, including but not limited to one containing 7 to 3000 mg, preferably 70 to 1400 mg of active ingredient per unit dosage form. An oral dosage of 50-1000 mg is usually convenient. Ideally the active ingredient should be administered to achieve peak plasma concentrations of the active compound of from about 1 μM to 100 mM or from 0.2 to 700 μM, or about 1.0 to 10 μM.

The concentration of active compound in the drug composition will depend on absorption, inactivation, and excretion rates of the drug as well as other factors known to those of skill in the art. It is to be noted that dosage values will also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed composition. The active ingredient may be administered at once, or may be divided into a number of smaller doses to be administered at varying intervals of time.

A preferred mode of administration of the active compound is oral. Oral compositions will generally include an inert diluent or an edible carrier. They may be enclosed in gelatin capsules or compressed into tablets. For the purpose of oral therapeutic administration, the active compound can be incorporated with excipients and used in the form of tablets, troches or capsules. Pharmaceutically compatible binding agents, and/or adjuvant materials can be included as part of the composition.

The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. When the dosage unit form is a capsule, it can contain, in addition to material of the above type, a liquid carrier such as a fatty oil. In addition, dosage unit forms can contain various other materials which modify the physical form of the dosage unit, for example, coatings of sugar, shellac, or other enteric agents.

The compound can be administered as a component of an elixir, suspension, syrup, wafer, chewing gum or the like. A syrup may contain, in addition to the active compounds, sucrose as a sweetening agent and certain preservatives, dyes and colorings and flavors.

The compound or a pharmaceutically acceptable prodrug or salts thereof can also be mixed with other active materials that do not impair the desired action, or with materials that supplement the desired action, such as antibiotics, antifungals, anti-inflammatories, or antiviral compounds, or with additional chemotherapeutic agents. Solutions or suspensions used for parenteral, intradermal, subcutaneous, or topical application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parental preparation can be enclosed in ampoules, disposable syringes or multiple dose vials made of glass or plastic.

In a preferred embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters and polylactic acid. Methods for preparation of such formulations will be apparent to those skilled in the art. The materials can also be obtained commercially from Alza Corporation. If administered intravenously, preferred carriers are physiological saline or phosphate buffered saline (PBS).

Liposomal suspensions (including liposomes targeted to infected cells with monoclonal antibodies to viral antigens) are also preferred as pharmaceutically acceptable carriers. These may be prepared according to methods known to those skilled in the art, for example, as described in U.S. Pat. No. 4,522,811 (which is incorporated herein by reference in its entirety). For example, liposome formulations may be prepared by dissolving appropriate lipid(s) (such as stearoyl phosphatidyl ethanolamine, stearoyl phosphatidyl choline, arachadoyl phosphatidyl choline, and cholesterol) in an inorganic solvent that is then evaporated, leaving behind a thin film of dried lipid on the surface of the container. An aqueous solution of the active compound or its monophosphate, diphosphate, and/or triphosphate derivatives is then introduced into the container. The container is then swirled by hand to free lipid material from the sides of the container and to disperse lipid aggregates, thereby forming the liposomal suspension.

Combination and Alternation Therapy

In one embodiment, the compounds described herein are administered in combination or alternation with another active compound.

In another embodiment, the second active compound is a compound that is used as an anti-HIV agent, including but not limited to a nucleoside or nonnucleoside reverse transcriptase inhibitor, a protease inhibitor, a fusion inhibitor, cytokine and interferon. The compound provided in combination or alternation can, as a nonlimiting example, be selected from the following lists:

| Brand Name | Generic Name |
|---|---|
| Agenerase | amprenavir |
| Combivir | lamivudine and zidovudine |
| Crixivan | indinavir, IDV, MK-639 |
| Emtriva | FTC, emtricitabine |
| Epivir | lamivudine, 3TC |
| Epzicom | abacavir/lamivudine |
| Fortovase | saquinavir |
| Fuzeon | enfuvirtide, T-20 |
| Hivid | zalcitabine, ddC, dideoxycytidine |
| Invirase | saquinavir mesylate, SQV |
| Kaletra | lopinavir and ritonavir |
| Lexiva | Fosamprenavir Calcium |
| Norvir | ritonavir, ABT-538 |
| Rescriptor | delavirdine, DLV |
| Retrovir | zidovudine, AZT, azidothymidine, ZDV |
| Reyataz | atazanavir sulfate |
| Sustiva | efavirenz |
| Trizivir | abacavir, zidovudine, and lamivudine |
| Truvada | tenofovir disoproxil/emtricitabine |
| Videx EC | enteric coated didanosine |
| Videx | didanosine, ddI, dideoxyinosine |
| Viracept | nelfinavir mesylate, NFV |
| Viramune | nevirapine, BI-RG-587 |
| Viread | tenofovir disoproxil fumarate |
| Zerit | stavudine, d4T |
| Ziagen | abacavir |

Further active agents include: GW5634 (GSK), (+)Calanolide A (Sarawak Med.), Capravirine (Agouron), MIV-150 (Medivir/Chiron), TMC125 (Tibotec), RO033-4649 (Roche), TMC114 (Tibotec), Tipranavir (B-I), GW640385 (GSK/Vertex), Elvucitabine (Achillion Ph.), Alovudine (FLT) (B-I), MIV-210 (GSK/Medivir), Racivir (Pharmasset), SPD754 (Shire Pharm.), Reverset (Incyte Corp.), FP21399 (Fuji Pharm.), AMD070 (AnorMed), GW873140 (GSK), BMS-488043 (BMS), Schering C/D (417690), PRO 542 (Progenics Pharm), TAK-220 (Takeda), TNX-355 (Tanox), UK-427,857 (Pfizer).

Further active agents include: Attachment and Fusion Inhibitors (i.e. AMD070, BMS-488043, FP21399, GW873140, PRO 542, Schering C, SCH417690, TAK-220, TNX-355 and UK-427,857); Integrase Inhibitors; Maturation Inhibitors (i.e. PA457); Zinc Finger Inhibitors (i.e. azodicarbonamide (ADA)); Antisense Drugs (i.e. HGTV43 by Enzo Therapeutics, GEM92 by Hybridon); Immune Stimulators (i.e. Ampligen by Hemispherx Biopharma, IL-2 (Proleukin) by Chiron Corporation, Bay 50-4798 by Bayer Corporation, Multikine by Cel-Sci Corporation, IR103 combo); Vaccine-Like Treatment (i.e. HRG214 by Virionyx, DermaVir, VIR201 (Phase I/IIa)).

In one embodiment, the compounds of the invention are administered in combination with another active agent. The compounds can also be administered concurrently with the other active agent. In this case, the compounds can be administered in the same formulation or in a separate formulation. There is no requirement that the compounds be administered in the same manner. For example, the second active agent can be administered via intravenous injection while the compounds of the invention may be administered orally. In another embodiment, the compounds of the invention are administered in alternation with at least one other active compound. In a separate embodiment, the compounds of the invention are administered during treatment with an active agent, such as, for example, an agent listed above, and administration of the compounds of the invention is continued after cessation of administration of the other active compound.

The compounds of the invention can be administered prior to or after cessation of administration of another active compound. In certain cases, the compounds may be administered before beginning a course of treatment for viral infection or for secondary disease associated with HIV infections, for example. In a separate embodiment, the compounds can be administered after a course of treatment to reduce recurrence of viral infections.

In another embodiment, the active compound is a compound that is used as a chemotherapeutic. A compound provided in combination or alternation can, for example, be selected from the following list:

| | | | |
|---|---|---|---|
| 13-cis-Retinoic | 2-Amino-6- | 2-CdA | 2- |
| Acid | Mercaptopurine | | Chlorodeoxyadenosine |
| 5-fluorouracil | 5-FU | 6-TG | 6-Thioguanine |
| 6-Mercaptopurine | 6-MP | Accutane | Actinomycin-D |
| Adriamycin | Adrucil | Agrylin | Ala-Cort |
| Aldesleukin | Alemtuzumab | Alitretinoin | Alkaban-AQ |
| Alkeran | All-transretinoic | Alpha interferon | Altretamine |
| | acid | | |
| Amethopterin | Amifostine | Aminoglutethimide | Anagrelide |
| Anandron | Anastrozole | Arabinosylcytosine | Ara-C |
| Aranesp | Aredia | Arimidex | Aromasin |
| Arsenic trioxide | Asparaginase | ATRA | Avastin |
| BCG | BCNU | Bevacizumab | Bexarotene |
| Bicalutamide | BiCNU | Blenoxane | Bleomycin |
| Bortezomib | Busulfan | Busulfex | C225 |
| Calcium | Campath | Camptosar | Camptothecin-11 |
| Leucovorin | | | |
| Capecitabine | Carac | Carboplatin | Carmustine |
| Carmustine wafer | Casodex | CCNU | CDDP |
| CeeNU | Cerubidine | cetuximab | Chlorambucil |
| Cisplatin | Citrovorum | Cladribine | Cortisone |
| | Factor | | |
| Cosmegen | CPT-11 | Cyclophosphamide | Cytadren |
| Cytarabine | Cytarabine | Cytosar-U | Cytoxan |
| | liposomal | | |
| Dacarbazine | Dactinomycin | Darbepoetin alfa | Daunomycin |
| Daunorubicin | Daunorubicin | Daunorubicin | DaunoXome |
| | hydrochloride | liposomal | |
| Decadron | Delta-Cortef | Deltasone | Denileukin diftitox |
| DepoCyt | Dexamethasone | Dexamethason | dexamethasone |
| | | Acetate | sodium phosphate |
| Dexasone | Dexrazoxane | DHAD | DIC |
| Diodex | Docetaxel | Doxil | Doxorubicin |
| Doxorubicin | Droxia | DTIC | DTIC-Dome |
| liposomal | | | |
| Duralone | Efudex | Eligard | Ellence |
| Eloxatin | Elspar | Emcyt | Epirubicin |
| Epoetin alfa | Erbitux | Erwinia-L- | Estramustine |
| | | asparaginase | |
| Ethyol | Etopophos | Etoposide | Etoposide |
| | | | phosphate |
| Eulexin | Evista | Exemestane | Fareston |
| Faslodex | Femara | Filgrastim | Floxuridine |
| Fludara | Fludarabine | Fluoroplex | Fluorouracil |
| Fluorouracil | Fluoxyrnesterone | Flutamide | Folinic Acid |
| (cream) | | | |
| FUDR | Fulvestrant | G-CSF | Gefitinib |
| Gemcitabine | Gemtuzumab | Gemzar | Gleevec |
| | ozogamicin | | |
| Gliadel wafer | Glivec | GM-CSF | Goserelin |
| granulocyte colony | Granulocyte | Halotestin | Herceptin |
| stimulating factor | macrophage | | |
| | colony | | |
| | stimulating factor | | |
| Hexadrol | Hexalen | Hexamethylmelamine | HMM |
| Hycamtin | Hydrea | Hydrocort Acetate | Hydrocortisone |
| Hydrocortisone | Hydrocortisone | Hydrocortone | Hydroxyurea |
| sodium phosphate | sodium succinate | phosphate | |
| Ibritumomab | Ibritumomab | Idamycin | Idarubicin |
| | Tiuxetan | | |
| Ifex | IFN-alpha | Ifosfamide | IL-2 |
| IL-11 | Imatinib | Imidazole | Interferon alfa |
| | mesylate | Carboxamide | |
| Interferon Alfa-2b | Interleukin-2 | Interleukin-11 | Intron A |
| (PEG conjugate) | | | (interferon |
| | | | alfaL2b) |
| Iressa | Irinotecan | Isotretinoin | Kidrolase |
| Lanacort | L-asparaginase | LCR | Letrozole |
| Leucovorin | Leukeran | Leukine | Leuprolide |
| Leurocristine | Leustatin | Liposomal Ara-C | Liquid Pred |
| Lomustine | L-PAM | L-Sarcolysin | Lupron |
| Lupron Depot | Matulane | Maxidex | Mechlorethamine |
| Mechlorethamine | Medralone | Medrol | Megace |
| hydrochloride | | | |
| Megestrol | Megestrol | Melphalan | Mercaptopurine |
| | Acetate | | |
| Mesna | Mesnex | Methotrexate | Methotrexate |
| | | | Sodium |
| Methylprednisolone | Meticorten | Mitomycin | Mitomycin-C |
| Mitoxantrone | M-Prednisol | MTC | MTX |
| Mylocel | Mylotarg | Navelbine | Neosar |
| Neulasta | Neumega | Neupogen | Nilandron |
| Nilutamide | Nitrogen Mustard | Novaldex | Novantrone |
| Octreotide | Octreotide | Oncospar | Oncovin |
| | acetate | | |
| Ontak | Onxal | Oprevelkin | Orapred |
| Orasone | Oxaliplatin | Paclitaxel | Pamidronate |
| Panretin | Paraplatin | Pediapred | PEG Interferon |
| Pegaspargase | Pegfilgrastim | PEG-INTRON | PEG-L- |
| | | | asparaginase |
| Phenylalanine | Platinol | Platinol-AQ | Prednisolone |
| Mustard | | | |
| Prednisone | Prelone | Procarbazine | PROCRIT |
| Proleukin | Prolifeprospan 20 | Purinethol | Raloxifene |
| | with Carmustine | | |
| | implant | | |
| Rheumatrex | Rituxan | Rituximab | Roveron-A |
| | | | (interferon α-2a) |
| Rubex | Rubidomycin | Sandostatin | Sandostatin LAR |
| | hydrochloride | | |
| Sargramostim | Solu-Cortef | Solu-Medrol | STI-571 |
| Streptozocin | Tamoxifen | Targretin | Taxol |
| Taxotere | Temodar | Temozolomide | Teniposide |
| TESPA | Thalidomide | Thalomid | TheraCys |
| Thioguanine | Thioguanine | Thiophosphoamide | Thioplex |
| | Tabloid | | |
| Thiotepa | TICE | Toposar | Topotecan |
| Toremifene | Trastuzumab | Tretinoin | Trexall |
| Trisenox | TSPA | VCR | Velban |
| Velcade | VePesid | Vesanoid | Viadur |
| Vinorelbine | Vinorelbine | VLB | VM-26 |
| | tartrate | | |
| VP-16 | Vumon | Xeloda | Zanosar |
| Zevalin | Zinecard | Zoladex | Zoledronic acid |
| Zometa | | | |

Diseases

The compounds described herein, are particularly useful for the treatment or prevention of a disorder associated with chemokine receptor binding or activation, and particularly HIV viral infections. However, numerous other diseases have been associated with chemokine receptor signaling.

Human and simian immunodeficiency viruses (HIV and SIV, respectively) enter cells through a fusion reaction triggered by the viral envelope glycoprotein (Env) and two cellular molecules: CD4 and a chemokine receptor, generally either CCR5 or CXCR5. (Alkhatib G, Combadiere C, Croder C, Feng Y, Kennedy P E, Murphy P M, Berger E A. CC CKR5. a RANTES, MIP-1apha, MIP-1Beta receptor as a fusion cofactor for macrophage-tropic HIV-1. Science. 1996; 272: 1955-1988).

In approximately 50% of infected individuals, CXCR4-tropic (X4-tropic) viruses emerge later in HIV infection, and their appearance correlates with a more rapid CD4 decline and a faster progression to AIDS (Connor, et al. (1997) J Exp. Med. 185: 621-628). Dualtropic isolates that are able to use both CCR5 and CXCR4 are also seen and may represent intermediates in the switch from CCR5 to CXCR4 tropism (Doranz, et al. (1996) Cell. 85: 1149-1158).

In a separate embodiment, a method for the treatment of, prevention of, or reduced severity of liver disease associated with viral infections including administering at least one compound described herein is provided.

Chronic hepatitis C virus (HCV) and hepatitis B virus (HBC) infection is accompanied by inflammation and fibrosis eventually leading to cirrhosis. A study testing the expression and function of CXCR4 on liver-infiltrating lymphocytes (LIL) revealed an important role for the CXCL12/CXCR4 pathway in recruitment and retention of immune cells in the liver during chronic HCV and HBV infection (Wald, et al. (2004) European Journal of Immunology. 34(4): 1164-1174).

High levels of CXCR4 and TGF-. have been detected in liver samples obtained from patients infected with HCV. (Mitra, et al. (1999) Int. J. Oncol. 14: 917-925). In vitro, TGF-. has been shown to up-regulate the expression of CXCR4 on naïve T cells and to increase their migration. The CD69/TGF-./CXCR4 pathway may be involved in the retention of recently activated lymphocytes in the liver (Wald, et al. European Journal of Immunology. 2004; 34(4): 1164-1174).

### EXAMPLES

### Preparation of Compounds of Formula (IA)

Compounds of Formula (IA), below:

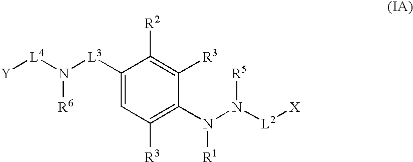

can be prepared by the general methods described below in General Scheme A.

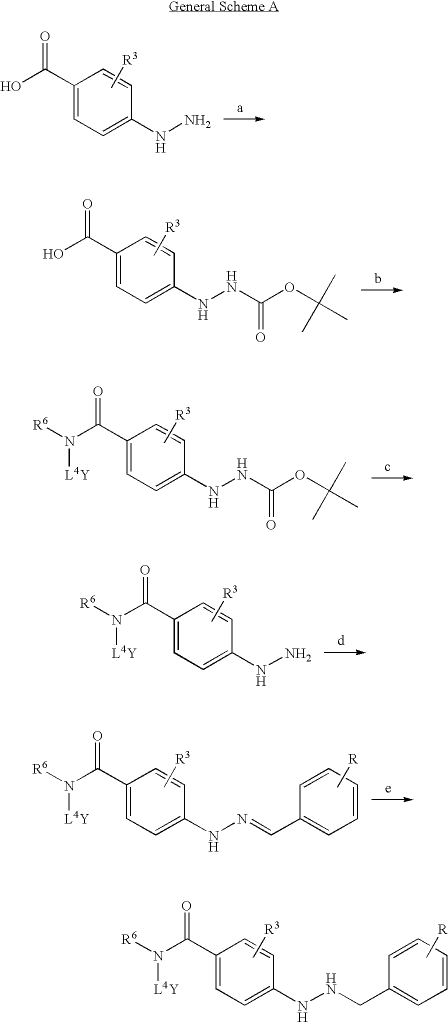

Reagents and conditions: (a) di-tert-butyl dicarboxylate, Et₃N, THF; (b) amine, EDAC, HOBt, ⁱPr₂NEt, DMF; (c) trifluoroacetic acid, CH₂Cl₂; (d) aldehyde, catalytic AcOH, 1,2-dichloroethane, 60° C.; (e) H₂, Pt/C, HCl/MeOH, r.t. to 80° C.

For example, compounds in which L³ is —C(O)— can be prepared by reacting an appropriately substituted 4-hydrazinylbenzoic acid with di-tert-butyl dicarboxylate to form a 4-(2-(tert-butoxycarbonyl)hydrazinyl)benzoic acid (step a). The 4-(2-(tert-butoxycarbonyl)hydrazinyl)benzoic acid can then be converted to a tert-butyl 2-(4-(carbamoyl)phenyl)hydrazinecarboxylate by reaction with an amine (step b). The tert-butoxycarbonyl protecting group can then be removed by treatment with trifluoroacetic acid (step c), and the resulting hydrazinylbenzamide can then be converted to a 4-(2-arylidenehydrazinyl)-N,N-dimethylbenzamide by reaction with an aryl aldehyde (step d), which can subsequently be reduced (e.g., by catalytic hydrogenation) to the corresponding 4-(2-benzylhydrazinyl)-N,N-dimethylbenzamide.

It will be recognized that this general procedure can be modified in numerous ways to provide the compounds of the present invention, for example by varying the substitution of the starting hydrazinylbenzoic acid (or using an analogous benzene sulfonic acid), varying the amine used in step b, varying the aldehyde used in step d, omitting the reduction in step e, reducing the amide, etc.

### Preparation of Compounds of Formula (IB)

Compounds of Formula (IB), below:

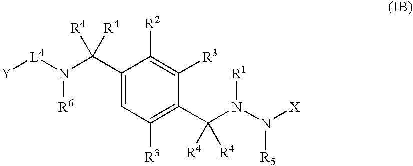

can be prepared by the general methods described below in General Scheme B.

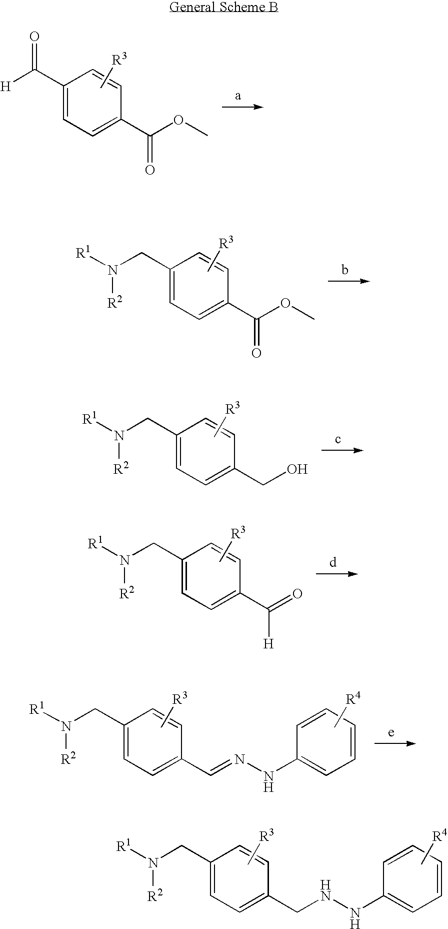

Reagents and conditions: (a) amine, Na(OAc)₃BH, catalytic AcOH, 1,2-dichloroethane; (b) lithium borohydride, methanol; (c) manganese dioxide, CH₂Cl₂; (d) phenylhydrazine, catalytic AcOH, 1,2-dichloroethane, 60° C.; (e) H₂, Pt/C, HCl/MeOH, r.t. to 80° C.

For example, compounds of formula (IB) can be prepared by reacting a substituted or unsubstituted formylbenzoate ester with an amine to form an aminomethylbenzoate (step a), which can then be reduced to an aminomethylbenzylic alcohol (step b). The benzylic alcohol can then be oxidized to the corresponding aminomethylbenzaldehyde (step c). The aminomethylbenzaldehyde can then be reacted with an appropriately substituted hydrazine to form a hydrazone (step d), which can optionally be reduced to a substituted hydrazine (step e).

It will be recognized that this general procedure can be modified in numerous ways to provide the compounds of the present invention, for example by varying the substitution of the starting formylbenzoate ester (e.g., using a substituted or unsubstituted 4-keto benzoate), converting the aminomethyl benzoate, benzyl alcohol or aldehyde formed after steps a, b, or c, respectively, to an aminomethyl ketone, varying the amine used in step a, varying the substitution of the hydrazine in step d, etc.

### Compounds of Category I

### First Aspect

Compounds of formula IC, wherein R⁵ and L²-X, together with the nitrogen atom to which they are shown attached form a heterocyclic ring, can have the following general structure IC-1:

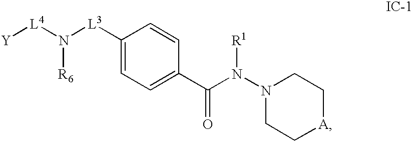

wherein Y-L⁴-N(R⁶)—, L³, R¹ and A are defined herein below in Table 1.

TABLE 1

| Cmpd. | Y-L⁴-N(R⁶)- | L³ | R¹ | A |
|---|---|---|---|---|
| A | 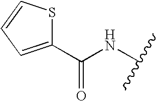 | CH₂ | Benzyl | O |
| B | 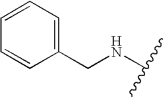 | CH₂ | Benzyl | O |
| C | 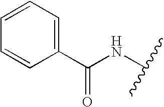 | CH₂ | Benzyl | O |
| D | 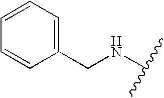 | CH₂ | 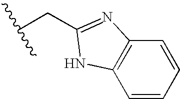 | O |
| E | 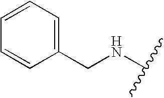 | CO | 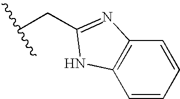 | O |
| F | 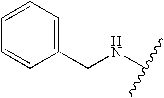 | CH₂ | benzyl | NMe |
| G | 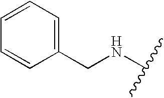 | CO | benzyl | NMe |
| H | 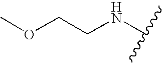 | CO | benzyl | NMe |
| I | 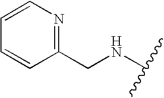 | CH₂ | H | O |
| J | 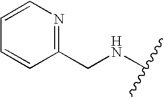 | CO | H | O |
| K | 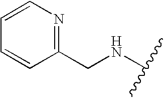 | CH₂ | benzyl | O |
| L | 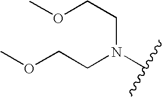 | CO | benzyl | O |
| M | 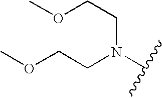 | CH₂ | benzyl | O |
| N | 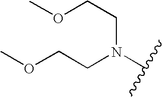 | CH₂ | 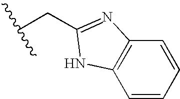 | O |
| O | 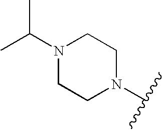 | CH₂ | 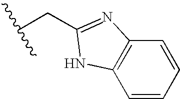 | O |
| P | 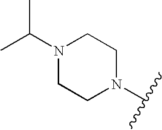 | CO | 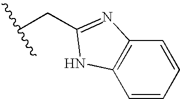 | O |
| Q | 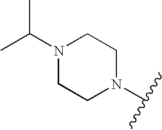 | CH₂ | H | O |
| R | 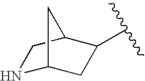 | CH₂ | benzyl | NMe |
| S | 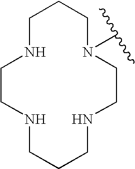 | CO | benzyl | O |
| T | 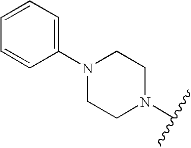 | CH₂ | benzyl | O |
| U | 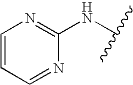 | CH₂ | benzyl | O |
| V | 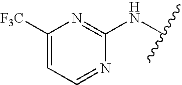 | CH₂ | benzyl | O |
| W | 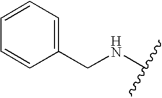 | CH₂ | 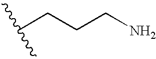 | O |
| X | 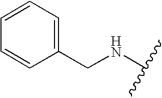 | CH₂ | 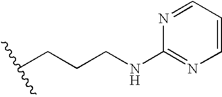 | O |
| Y | 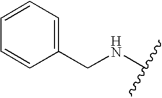 | CH₂ | 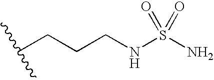 | O |

The compounds which comprise the first aspect of Embodiment I of the present invention can be prepared by the procedures outlined herein below in Schemes I and II.

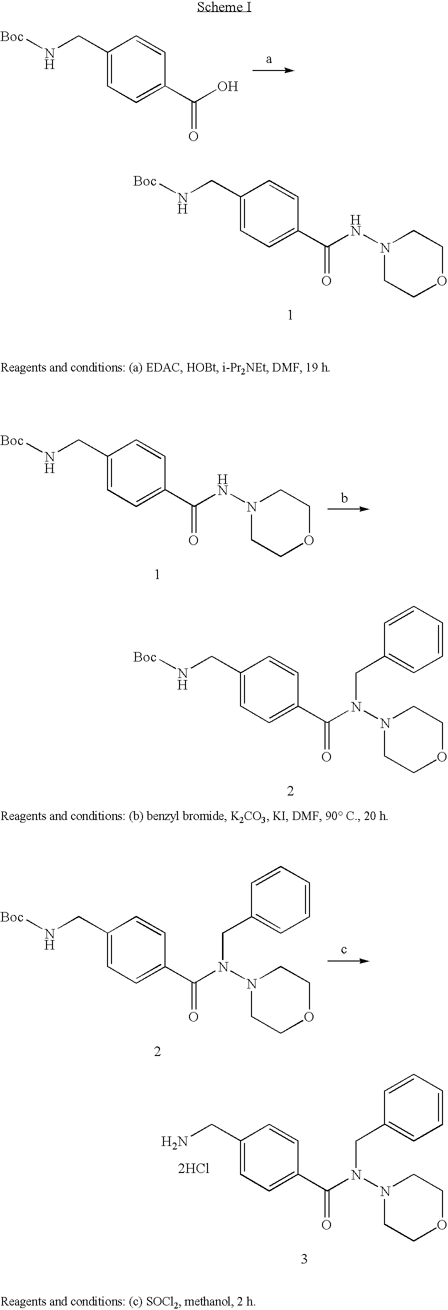

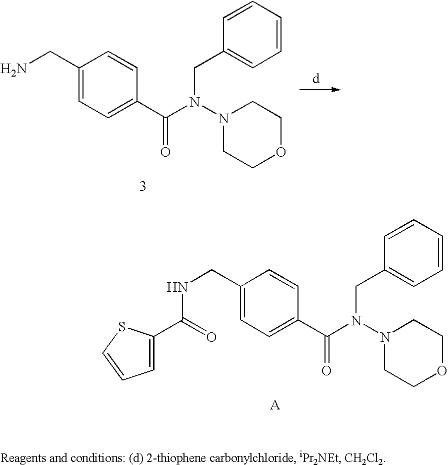

### Example 1

### N-(4-(morpholinocarbamoyl)benzyl)thiophene-2-carboxamide (A)

Preparation of tert-butyl 4-(morpholinocarbamoyl)benzylcarbamate (1): To a cold (0° C.) solution of 4-((tert-butoxycarbonyl)methyl)benzoic acid (2.0 g, 8.0 mmol; Fluka), 1-hydroxybenzotriazole hydrate (1.3 g, 5.7 mmol) and N,N-diisopropylethylamine (9.3 mL, 86.2 mmol) in DMF (60 mL) was added N-(3-dimethylaminopropyl)-N′-ethylcarbodiimide hydrochloride (“EDAC”; 1.8 g, 5.7 mmol). After stirring the reaction at 0° C. for 1 hour, 4-aminomorpholine (1.3 mL, 12.7 mmol) was added to the mixture and the reaction was warmed to room temperature. After stirring for 19 hours, the solution was poured into aqueous saturated NaHCO₃ and extracted with EtOAc. The organic phase was washed three times with H₂O, dried (MgSO₄), filtered and concentrated in vacuo. The residue was purified by silica gel chromatography (2% methanol/CHCl₃ to 20% methanol/CHCl₃) to afford 2.1 g of the desired product as a white solid: ¹H NMR (300 MHz, d₆-DMSO) δ 9.45 (s, 1H), 7.71 (d, J=8.0 Hz, 2H), 7.47 (t, J=6.0 Hz, 1H), 7.28 (d, J=8.0 Hz, 2H), 4.15 (d, J=6.0 Hz, 2H), 3.66 (m, 4H), 2.86 (m, 4H), 1.39 (s, 9H).

Preparation of tert-butyl 4-(benzyl(morpholino)carbamoyl)benzylcarbamate (2): To a solution of tert-butyl 4-(morpholinocarbamoyl)benzylcarbamate, 1, (0.55 g, 1.64 mmol), K₂CO₃ (0.50 g, 3.61 mmol) and potassium iodide (0.27 g, 1.64 mmol) in DMF (6 mL) was added benzyl bromide (0.43 mL, 3.61 mmol). The reaction was warmed to 90° C. and stirred for 20 hour. The reaction mixture was poured into aqueous saturated NH₄Cl and extracted with EtOAc. The organic phase was washed with brine, dried (MgSO₄), filtered and concentrated in vacuo. The residue was purified by silica gel chromatography (100% CH₂Cl₂ to 10% methanol/CH₂Cl₂) to afford 450 mg of the desired product: ¹H NMR (400 MHz, d₆-DMSO) δ 7.77 (d, J=8.0 Hz, 2H), 7.42-7.32 (m, 6H), 7.13 (d, J=8.0 Hz, 2H), 5.15 (s, 2H), 4.11 (d, J=6.0 Hz, 2H), 4.02-3.95 (m, 4H), 3.76 (d, J=12.0 Hz, 2H), 3.44-3.35 (m, 2H), 1.39 (s, 9H).

Preparation of 4-(aminomethyl)-N-benzyl-N-morpholinobenzamide dihydrochloride (3): To a solution of tert-butyl 4-(benzyl(morpholino)carbamoyl)benzylcarbamate, 2, (0.4 g, 1.0 mmol) in methanol (20 mL) was added dropwise thionyl chloride (1.0 mL). After stirring 2 hours at room temperature, the reaction mixture was concentrated in vacuo to afford 0.5 g of the hydrochloride salt as a yellow solid, which was used without further purification: ¹H NMR (400 MHz, d₆-DMSO) δ 11.62 (s, 1H), 8.70 (bd s, 3H, NH3), 7.91 (d, J=7.2 Hz, 2H), 7.68 (m, 2H), 7.49-7.37 (m, 4H), 5.39-5.25 (m, 2H), 4.12-4.00 (m, 8H), 3.81-3.55 (m, 2H).

Preparation of N-(4-(benzyl(morpholino)carbamoyl)benzyl)thiophene-2-carboxamide (A): To a solution of 4-(aminomethyl)-N-benzyl-N-morpholinobenzamide, 3, (0.22 g, 0.55 mmol) and N,N-diisopropylethylamine (0.48 mL, 2.75 mmol) in CH₂Cl₂ (5 mL) was added 2-thiophene carbonyl chloride (0.07 mL, 0.61 mmol). After stirring the reaction for 1.5 hours at room temperature, the solution was poured into aqueous saturated NaHCO₃ and extracted with EtOAc. The organic phase was dried (MgSO₄), filtered and concentrated in vacuo. The residue was purified by silica gel chromatography (CH₂Cl₂ to 10% methanol/CH₂Cl₂) to afford 153 mg of the desired product: ¹H NMR (400 MHz, d₆-DMSO) δ 9.06 (t, J=6.4 Hz, 1H), 7.81-7.76 (m, 4H), 7.38-7.30 (m, 5H), 7.22 (d, J=8.0 Hz, 2H), 7.16 (t, J=4.0 Hz, 1H), 5.15 (s, 2H), 4.44 (d, J=6.4 Hz, 2H), 3.89 (dd, J=11.6, 11.6 Hz, 4H), 3.76 (d, J=12.0 Hz, 2H), 3.42 (dd, J=9.2, 9.2 Hz, 2H); ESI+ MS: m/z (rel intensity) 436 (100, M+H).

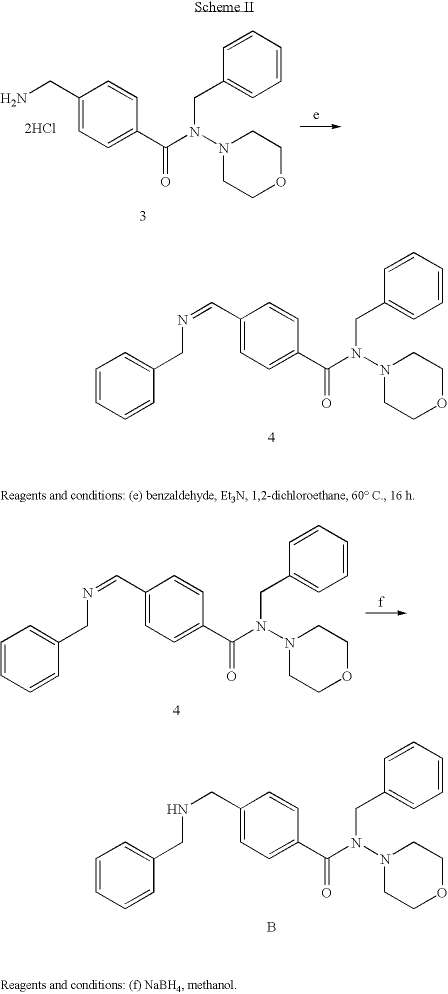

### Example 2

Preparation of N-benzyl-4-((benzylimino)methyl)-N-morpholinobenzamide (4): To a solution of 4-(aminomethyl)-N-benzyl-N-morpholinobenzamide dihydrochloride, 3, (0.25 g, 0.63 mmol) and benzaldehyde (0.07 mL, 0.63 mmol) in 1,2-dichloroethane (3 mL) was added glacial acetic acid (1 drop). The reaction mixture was heated to 60° C. After 16 hour, no reaction was observed. Triethylamine (0.35 mL, 1.89 mmol) was added to the mixture, and heating was continued for 1.5 hours. The reaction mixture was poured into aqueous saturated NaHCO₃ and extracted with EtOAc. The organic phase was dried (MgSO₄), filtered and concentrated in vacuo. The residue (100 mg) was used without further purification.

Preparation of N-benzyl-4-((benzylamino)methyl)-N-morpholinobenzamide (B): To a solution of N-benzyl-4-((benzylimino)methyl)-N-morpholinobenzamide, 4, (0.090 g, 0.22 mmol) in methanol (3 mL) was added sodium borohydride (0.01 g, 0.33 mmol). After stirring the reaction for 1.5 hours at room temperature, the mixture was poured into aqueous saturated NaHCO₃ and extracted with 20% isopropanol/CHCl₃. The combined organic phases were dried (MgSO₄), filtered and concentrated in vacuo. The residue was purified by silica gel chromatography (100% CHCl₃ to 10% methanol/CHCl₃) to afford 63 mg of the desired product: ¹H NMR (400 MHz, d₆-DMSO) δ 7.77 (d, J=8.4 Hz, 2H), 7.39-7.22 (m, 12H), 5.16 (s, 2H), 4.01 (dd, J=10.8, 10.8 Hz, 4H), 3.77 (d, J=12.0 Hz, 2H), 3.67 (s, 4H), 3.42 (ddd, J=11.6, 3.2, 3.2 Hz, 2H); ESI+ MS: m/z (rel intensity) 416 (100, M+H).

Compounds C, D, I, K, and W-Y can be prepared using methods similar to those described in Schemes I and II, above. For example, the benzyl bromide of step b of Scheme I can be replaced with the appropriate arylalkyl halide or heteroaryl alkyl halide (or step b can be amended when R¹ is H), the 2-thiophene carbonyl chloride of step d of Scheme I can be replaced with the appropriate arylacyl halide or heteroarylacyl halide, and benzaldehyde in step e of Scheme II can be replaced with the appropriate aryl or heteroaryl aldehyde. Compounds in which A is “NMe” (e.g., compound F) can be prepared by replacing 4-aminomorpholine in step a of Scheme I with 4-methylpiperazin-1-amine. Compounds in which L³ is —C(O)— (e.g., compounds E, G, H, and J) can be prepared by replacing the 4-((tert-butoxycarbonylamino)methyl)benzoic acid starting material of step a of Scheme I with 4-(tert-butoxycarbonylcarbamoyl)benzoic acid.

Compounds in which L³ is —CH₂— and Y-L⁴-N(R⁶)— is substituted alkylamino (e.g., compounds M-O, Q, R, and T, where R⁶ and/or Y-L⁴- is alkoxyalkyl) can be prepared by the general Scheme C shown below.

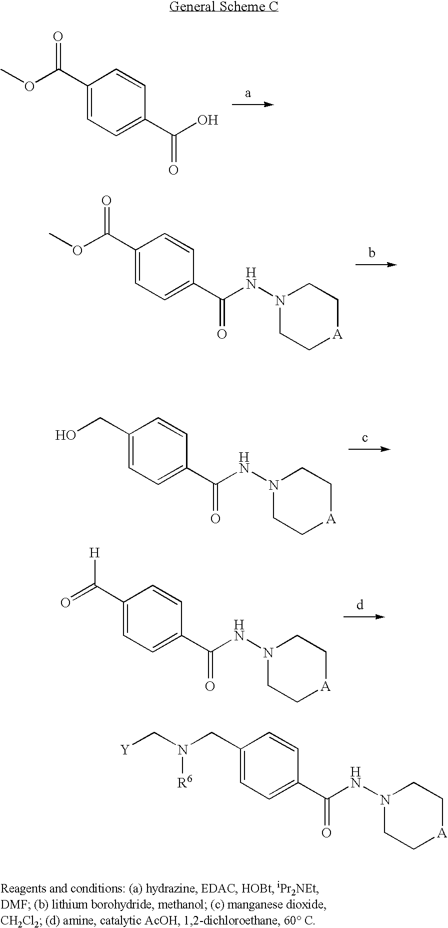

Compounds in which L³ is —CO— and Y-L⁴-N(R⁶)— is substituted alkylamino or substituted or unsubstituted heterocyclyl (e.g., compounds L, P and S) can be prepared by the general Scheme D shown below.

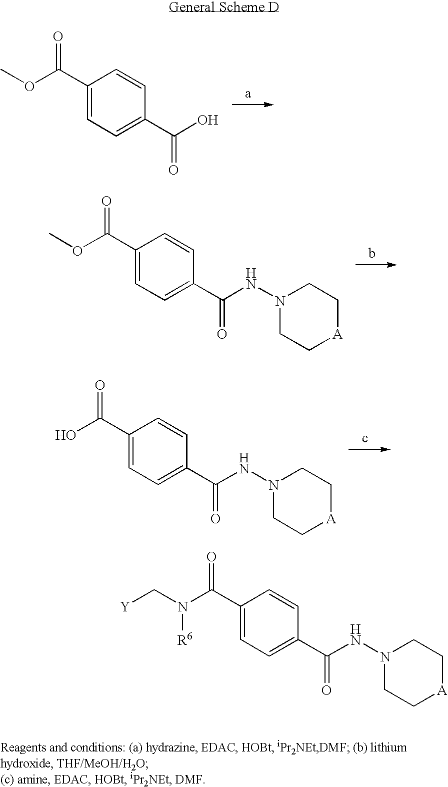

Compounds in which L³ is —CH₂— and Y-L⁴-N(R⁶)— is heteroarylamino (e.g., pyrimidylamino, compounds U and V) can be prepared by replacing the benzaldehyde starting material of step e of Scheme II with the appropriate 2-halopyrimidine and reacting them under basic conditions (ⁱPr₂NEt, DMF, room temperature to 100° C.).

Compounds in which L³ is —CH₂— and R¹ is substituted or unsubstituted alkylamino (e.g., compounds W, X and Y) can be prepared by the general Scheme E shown below. Reaction of the primary amine after step e with the appropriate electrophile (e.g., 2-chloropyrimidine or chlorosulfonylisocyanate) can provide a method for preparing compounds X and Y, and compounds similar thereto.

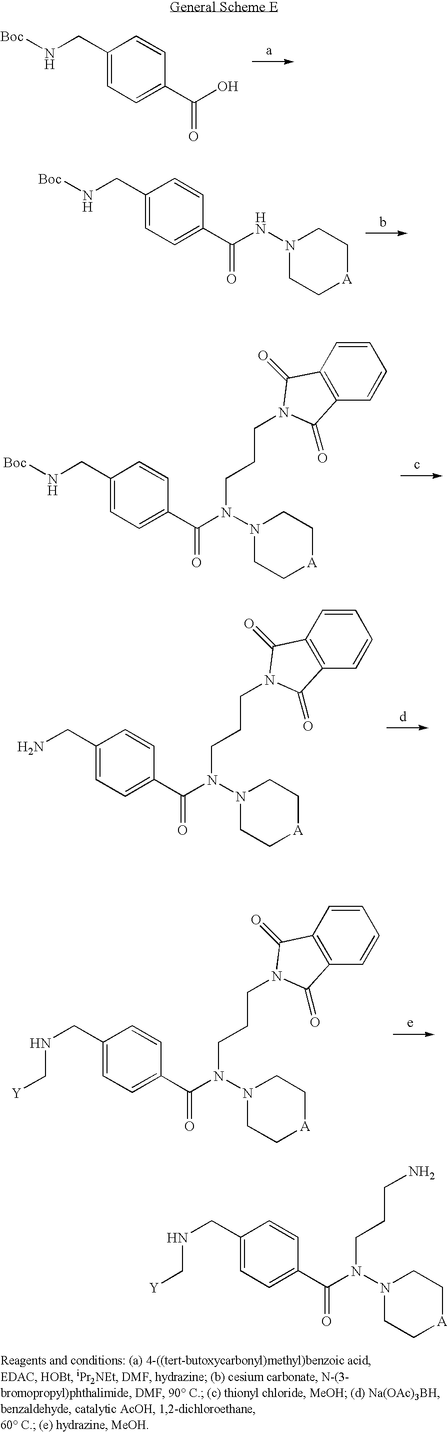

### Compounds of Category I

### Second Aspect

Compounds of formula IC-2, wherein Y-L⁴-N(R⁶)—, L³, and X are defined herein below in Table 2.

TABLE 2

| | |
|---|---|
| | IC-2 |
| 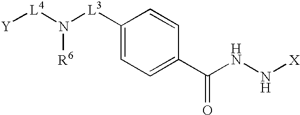 | |

| Cmpd. | Y-L⁴-N(R⁶)- | L³ | X |
|---|---|---|---|
| Z | 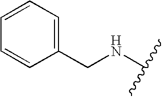 | CH₂ | 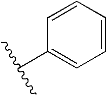 |
| AA | 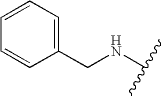 | CH₂ | 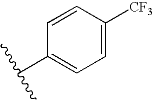 |
| AB | 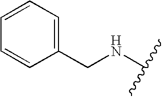 | CH₂ | 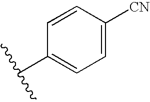 |
| AC | 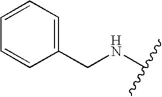 | CH₂ | 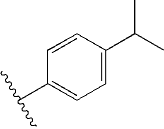 |
| AD | 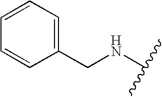 | CH₂ | 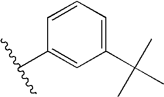 |
| AE | 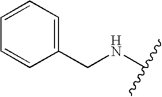 | CH₂ | 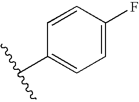 |
| AF | 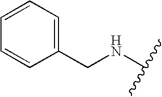 | CH₂ | 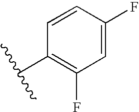 |
| AG | 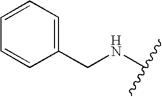 | CH₂ | 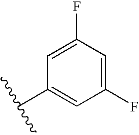 |
| AH | 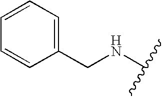 | CH₂ | 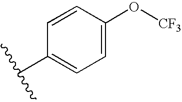 |
| AI | 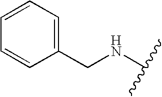 | CH₂ | 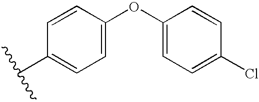 |
| AJ | 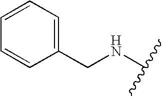 | CH₂ | 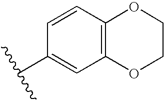 |
| AK | 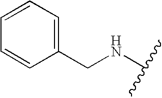 | CH₂ | 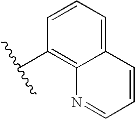 |
| AL | 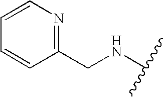 | CH₂ | 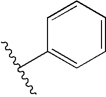 |
| AM | 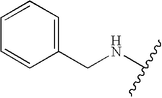 | CO | 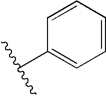 |
| AN | 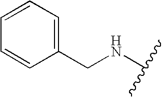 | CO | 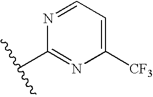 |
| AO | 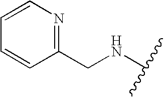 | CO | 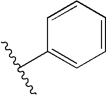 |
| AP | 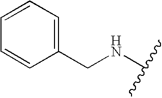 | CH₂ | 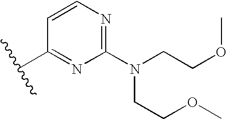 |
| AQ | 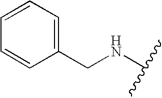 | CH₂ | 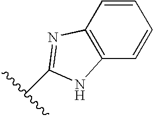 |
| AR | 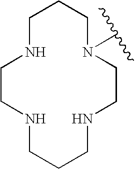 | CH₂ | 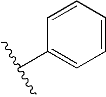 |
| AS | 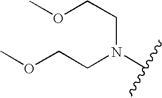 | CH₂ | 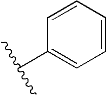 |
| AT | 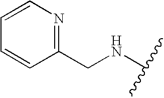 | CH₂ | 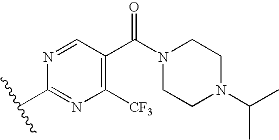 |
| AU | 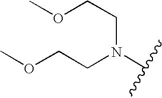 | CH₂ | 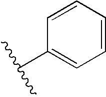 |
| AV | 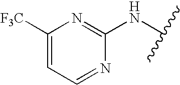 | CH₂ | 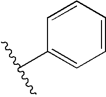 |
| AW | 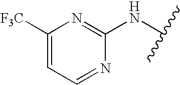 | CH₂ | 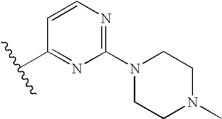 |
| AX | 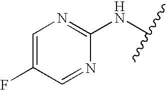 | CH₂ | 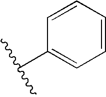 |
| AY | 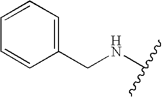 | CH₂ | 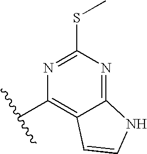 |
| AZ | 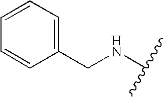 | CH₂ | 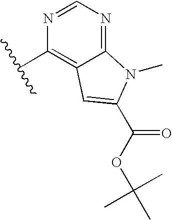 |
| BA | 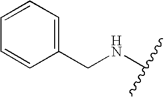 | CH₂ | 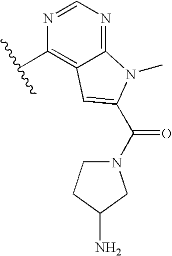 |
| BB | 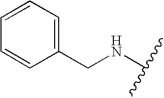 | CH₂ | 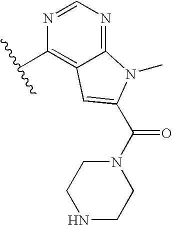 |
| BC | 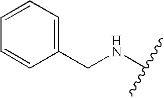 | CH₂ | 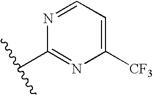 |
| BD | 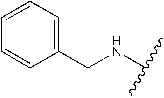 | CH₂ | 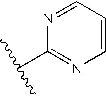 |
| BE | 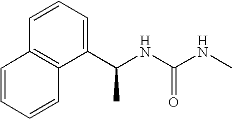 | CH₂ | 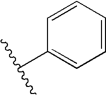 |

The compounds which comprise the second aspect of Category I of the present invention can be prepared by the procedure outlined herein below in Schemes III, IV, and V.

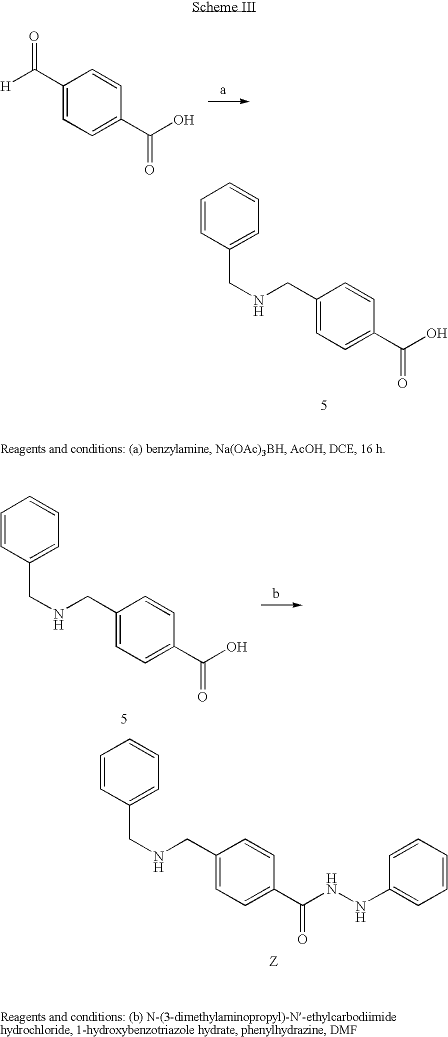

### Example 3

### 4-((benzylamino)methyl)-N′-phenylbenzohydrazide (Z)

Preparation of 4-((benzylamino)methyl)benzoic acid (5): To a solution of commercially available 4-formylbenzoic acid (3.4 g, 22.4 mmol) and benzylamine (2.5 mL, 22.4 mmol) in a 6:1 mixture of 1,2-dichloroethane/methanol (70 mL) at room temperature was added 5 drops of glacial acetic acid. After stirring the mixture for 15 min at room temperature, sodium triacetoxyborohydride (10.0 g, 47.1 mmol) was added and the mixture was heated to 60° C. for 16 hours. After the mixture was concentrated in vacuo, the crude residue was diluted with aqueous saturated NaHCO₃ and extracted with EtOAc. The insoluble solid was filtered off and dried under vacuum to afford 2.92 g of the desired carboxylic acid, 5. The organic layer was washed with brine, dried (MgSO₄), filtered and concentrated in vacuo. The crude residue was purified by preparative HPLC (Polaris C18 column using acetonitrile/water with 0.1% TFA) to afford 810 mg of acid 6: ¹H NMR (300 MHz, d₆-DMSO) δ 9.62 (s, br, NH), 7.98 (d, J=5.0 Hz, 2H), 7.60 (d, J=5.0 Hz, 2H), 7.48 (m, 2H), 7.42-7.40 (m, 3H); 4.26 (s, 2H), 4.19 (s, 2H); ESI⁺ MS: m/z (rel intensity) 242 (100, M+H).

Preparation of 4-((benzylamino)methyl)-N′-phenylbenzohydrazide (Z): To a solution of 4-((benzylamino)methyl)benzoic acid, 5, (0.15 g, 0.62 mmol), in DMF (2 mL) was added N-(3-dimethylaminopropyl)-N′-ethylcarbodiimide hydrochloride (0.18 g, 0.94 mmol) and 1-hydroxybenzotriazole hydrate (0.13 g, 0.94 mmol) and the mixture was stirred for 15 minutes at room temperature. Subsequently, phenyl hydrazine (0.19 mL, 1.88 mmol) was added in one portion followed by N-methylmorpholine (0.30 mL, 2.19 mmol). After stirring 17 hours at room temperature, the reaction mixture was diluted with methanol (10 mL). The resulting solution was purified by preparative HPLC (Polaris C18 column using acetonitrile/water with 0.1% TFA) to afford 60 mg of hydrazide Z: ¹H NMR (300 MHz, d₆-DMSO) δ 10.43 (s, NH), 9.53 (m, NH), 7.96 (d, J=5.0 Hz, 2H), 7.62 (d, J=5.0 Hz, 2H), 7.50-7.30 (m, 6H), 7.14 (m, 2H), 6.77 (m, 2H), 4.26 (s, 2H), 4.20 (s, 2H); ESI⁺ MS: m/z (rel intensity) 332 (100, M+H).

The following compounds AA-AI and AL were prepared using methods similar to those used to prepare compound Z (e.g., phenyl hydrazine in step b of Scheme III was replaced with the appropriately substituted hydrazine), and are non-limiting examples of the second aspect of Embodiment I.

4-((benzylamino)methyl)-N′-(4-(trifluoromethyl)phenyl)benzohydrazide (AA): ¹H NMR (300 MHz, d₆-DMSO) δ 10.6 (s, NH), 9.5 (m, NH), 7.95 (m, 2H), 7.66 (m, 2H), 7.5-7.3 (m, 6H), 6.86 (d, J=5.0 Hz, 2H), 6.78 (d, J=5.0 Hz, 2H), 4.22 (s, 2H), 4.14 (s, 2H); ESI⁺ MS: m/z (rel intensity) 400 (100, M+H).

4-((benzylamino)methyl)-N′-(4-cyanophenyl)benzohydrazide (AB): ¹H NMR (300 MHz, d₆-DMSO) δ 10.63 (s, NH), 9.85 (m, NH), 8.85 (m, NH), 7.96 (d, J=5.0 Hz, 2H), 7.69 (d, J=5.0 Hz, 2H), 7.57 (m, 4H), 7.41 (m, 3H), 6.82 (d, J=5.0 Hz, 2H), 4.22 (s, 2H), 4.14 (s, 2H); ESI⁺ MS: m/z (rel intensity) 357 (100, M+H).

4-((benzylamino)methyl)-N′-(4-isopropylphenyl)benzohydrazide (AC): ¹H NMR (300 MHz, d₆-DMSO) δ 10.43 (s, NH), 9.92 (m, NH), 7.92 (m, 2H), 7.68 (m, 2H), 7.57 (m, 2H), 7.41 (m, 2H), 7.00 (m, 2H), 6.73 (s, 2H), 6.60 (m, 2H), 4.22 (s, 2H), 4.14 (s, 2H), 2.74 (m, 1H), 1.14 (s, 6H); ESI⁺ MS: m/z (rel intensity) 374 (100, M+H).

4-((benzylamino)methyl)-N′-(3-tert-butylphenyl)benzohydrazide (AD): ¹H NMR (300 MHz, d₆-DMSO) δ 10.44 (s, NH), 9.79 (m, NH), 7.96 (m, 2H), 7.69 (m, 2H), 7.56 (m, 2H), 7.43 (m, 2H), 7.06 (m, 2H), 6.86 (s, 1H), 6.78 (m, 1H), 6.59 (m, 1H), 4.22 (s, 2H), 4.14 (s, 2H), 1.22 (s, 9H); ESI⁺ MS: m/z (rel intensity) 388 (100, M+H).

4-((benzylamino)methyl)-N′-(4-fluorophenyl)benzohydrazide (AE): ¹H NMR (300 MHz, d₆-DMSO) δ 10.5 (s, NH), 9.65 (m, NH), 7.96 (m, 2H), 7.69 (m, 2H), 7.53 (m, 2H), 7.43 (m, 3H), 6.99 (m, 2H), 6.86 (m, 2H), 4.22 (s, 2H), 4.14 (s, 2H); ESI⁺ MS: m/z (rel intensity) 350 (100, M+H).

4-((benzylamino)methyl)-N1′-(2,4-difluorophenyl)benzohydrazide (AF): ¹H NMR (300 MHz, d₆-DMSO) δ 10.6 (s, NH), 9.87 (m, NH), 7.96 (m, 2H), 7.69 (m, 2H), 7.57 (m, 2H), 7.43 (m, 3H), 7.2 (m, 2H), 6.86 (m, 2H), 4.22 (s, 2H), 4.14 (s, 2H); ESI⁺ MS: m/z (rel intensity) 368 (100, M+H).

4-((benzylamino)methyl)-N′-(3,5-difluorophenyl)benzohydrazide (AG): ¹H NMR (300 MHz, d₆-DMSO) δ 10.55 (s, NH), 9.81 (m, NH), 8.54 (m, 1H), 7.97 (d, J=5.0 Hz, 2H), 7.68 (d, J=5.0 Hz, 2H), 7.55 (m, 2H), 7.43 (m, 2H), 6.45 (m, 2H), 6.40 (m, 1H), 4.22 (s, 2H), 4.16 (s, 2H); ESI⁺ MS: m/z (rel intensity) 368 (100, M+H).

4-((benzylamino)methyl)-N′-(4-(trifluoromethoxy)phenyl)benzohydrazide (AH): ¹H NMR (300 MHz, d₆-DMSO) δ 7.96 (m, 2H), 7.70 (m, 2H), 7.58 (m, 2H), 7.43 (m, 3H), 7.17 (m, 2H), 6.84 (m, 2H), 4.22 (s, 2H), 4.14 (s, 2H); ESI⁺ MS: m/z (rel intensity) 416 (100, M+H).

4-((benzylamino)methyl)-N′-(4-(4-chlorophenoxy)phenyl)benzohydrazide (AI): ¹H NMR (300 MHz, d₆-DMSO) δ 10.48 (s, NH), 9.75 (m, NH), 7.97 (m, 2H), 7.68 (m, 2H), 7.54 (m, 2H), 7.43 (m, 5H), 7.36 (m, 1H), 6.91 (m, 5H), 4.22 (s, 2H), 4.14 (s, 2H); ESI⁺ MS: m/z (rel intensity) 458 (100, M+H).

N′-phenyl-4-((pyridin-2-ylmethylamino)methyl)benzohydrazide (AL): ¹H NMR (400 MHz, d₆-DMSO) δ 10.48 (s, NH), 9.75 (m, NH), 7.97 (m, 2H), 7.68 (m, 2H), 7.54 (m, 2H), 7.43 (m, 5H), 7.36 (m, 1H), 6.91 (m, 5H), 4.22 (s, 2H), 4.14 (s, 2H); ESI⁺ MS: m/z (rel intensity) 458 (100, M+H).

4-((benzylamino)methyl)-N′-(4-(trifluoromethyl)pyrimidin-2-yl)benzohydrazide (BC): ¹H NMR (400 MHz, d₆-DMSO) δ 8.56 (d, J=4.4 Hz, 1H), 7.77 (d, J=8.0 Hz, 2H), 7.35 (d, J=8.0 Hz, 2H), 7.34-7.20 (m, 5H), 6.98 (d, J=4.8 Hz, 1H), 3.80 (s, 2H), 3.77 (s, 2H), ESI⁺ MS: m/z (rel intensity) 402.2 (100, [M+H]⁺).

4-((benzylamino)methyl)-N′-(pyrimidin-2-yl)benzohydrazide (BD): ¹H NMR (400 MHz, d₆-DMSO) δ 8.32 (d, J=4.8 Hz, 1H), 7.80 (d, J=8.0 Hz, 2H), 7.34-7.20 (m, 7H), 6.66 (t, J=5.2 Hz, 2H), 3.75-3.72 (m, 4H), ESI⁺ MS: m/z (rel intensity) 334.2 (100, [M+H]⁺).

Similarly, compounds AP-BB could be prepared using methods similar to those described in Scheme III, using the appropriate amine or hydrazine instead of benzylamine and/or phenyl hydrazine in Scheme III.

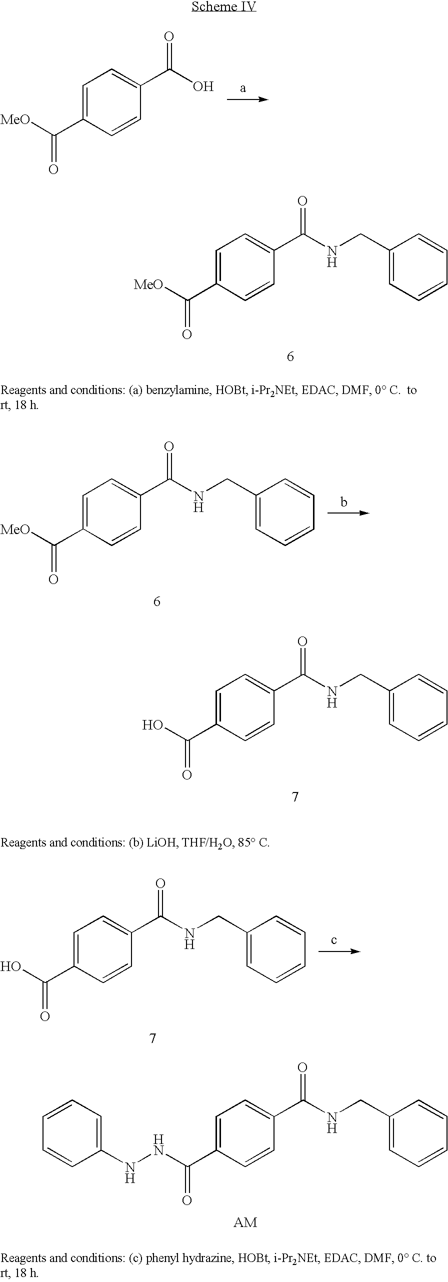

### Example 4

### 4-(carbamoyl)-N′-phenylbenzohydrazide (AM)

Preparation of methyl 4-(benzylcarbamoyl)benzoate (6): A solution of 4-(methoxycarbonyl)benzoic acid (2.00 g, 11.10 mmol) in DMF (20 mL) at 0° C. was treated with 1-hydroxybenzotriazole hydrate (1.80 g, 13.32 mmol), N,N-diisopropylethylamine (7.73 mL, 44.40 mmol) and N-(3-dimethylaminopropyl)-N′-ethylcarbodiimide hydrochloride (2.55 g, 13.32 mmol). The mixture was stirred at 0° C. for 1 hour. Benzylamine (2.42 mL, 22.20 mmol) was then added and the mixture was warmed to room temperature for 18 hours. The reaction mixture was quenched with saturated aqueous NaHCO₃ (10 mL) extracted three times with EtOAc (15 mL). The combined organic layers were washed with brine, dried (MgSO₄), filtered and concentrated in vacuo. The crude material was purified by silica gel chromatography (100% CHCl₃ to 10% methanol/CHCl₃) to afford 2.90 g of the desired product: ¹H NMR (400 MHz, CDCl₃) δ 8.07 (d, J=7.8 Hz, 2H), 7.83 (d, J=7.8 Hz, 2H), 7.40-7.20 (m, 5H), 6.49 (bs, 1H), 4.64 (d, J=4.4 Hz, 2H), 3.92 (s, 3H).

Preparation of 4-(benzylcarbamoyl)benzoic acid (7): A solution of methyl 4-(benzylcarbamoyl)benzoate, 6, (2.90 g, 11.10 mmol) in THF (15 mL) was treated with a solution of LiOH (1.07 g, 44.40 mmol) in water (15 ml). The resulting mixture was warmed to 85° C. and stirred at this temperature for 5 hours. After cooling to room temperature, the reaction mixture was extracted with EtOAc. The aqueous layer was acidified with 1N HCl to reach pH˜2 and the product was collected by filtration. The crude product (1.22 g) was used without further purification: ¹H NMR (400 MHz, d₆-DMSO) δ 9.19 (t, J=6.4 Hz, 1H), 8.01-7.93 (m, 4H), 7.31-7.15 (m, 5H), 4.45 (d, J=6.4 Hz, 2H); ESI⁻ MS: m/z (rel intensity) 254 (100, [M−H]).

Preparation of 4-(carbamoyl)-N′-phenylbenzohydrazide (AM): A solution of 4-(benzylcarbamoyl)benzoic acid, 79, (1.22 g, 4.77 mmol) in DMF (20 mL) at 0° C. was treated with 1-hydroxybenzotriazole hydrate (0.77 g, 5.73 mmol), N,N-diisopropylethylamine (3.33 mL, 19.11 mmol) and N-(3-dimethylaminopropyl)-N′-ethylcarbodiimide hydrochloride (1.09 g, 5.73 mmol). The resulting mixture was stirred at 0° C. for 1 hour. Phenyl hydrazine (0.94 mL, 9.55 mmol) was added and the reaction warmed to room temperature for 18 h. The reaction was quenched with saturated aqueous NaHCO₃ (20 mL) and extracted three times with EtOAc (15 mL). The combined organic layers were washed with brine (15 mL), dried (MgSO₄), filtered and concentrated in vacuo. The crude material was purified over silica (CHCl₃ to 10% methanol/CHCl₃) to afford 2.9 g of the desired product, compound AM: ¹H NMR (400 MHz, d₆-DMSO) δ 10.44 (d, J=2.8 Hz, 1H), 9.17 (m, 1H), 8.00-7.90 (m, 4H), 7.35-7.25 (m, 4H), 7.25-7.20 (m, 1H), 7.15-7.10 (m, 2H), 6.76 (d, J=7.6 Hz, 2H), 6.71-6.65 (m, 2H), 4.47 (d, J=6.0 Hz, 2H), ESI⁻ MS: m/z (rel intensity) 344 (100, [M−H]).

The following compounds AO and AN were prepared using methods similar to those used to prepare compound AM (e.g., for compound AO, pyridin-2-ylmethanamine was used in step a of Scheme IV instead of benzylamine; for compound AN, 2-hydrazinyl-4-(trifluoromethyl)pyrimidine was used instead of phenyl hydrazine in step c of Scheme IV), and are also non-limiting examples of the second aspect of Embodiment I.

4-(2-phenylhydrazinecarbonyl)-N-(pyridin-2-ylmethyl)benzamide (AO): ESI⁻ MS: m/z (rel intensity) 345 (100, [M−H]).

N-benzyl-4-(2-(4-(trifluoromethyl)pyrimidin-2-yl)hydrazinecarbonyl)benzamide (AN): ¹H NMR (400 MHz, d₆-DMSO) δ 10.68-10.60 (m, 1H), 9.80-9.76 (m, 1H), 9.22-9.15 (m, 1H), 8.80-8.65 (m, 1H), 8.05-7.90 (m, 4H), 7.38-7.30 (m, 4H), 7.30-7.20 (m, 2H), 4.47 (d, J=5.6 Hz, 2H), ESI⁻ MS: m/z (rel intensity) 414 (100, [M−H]).

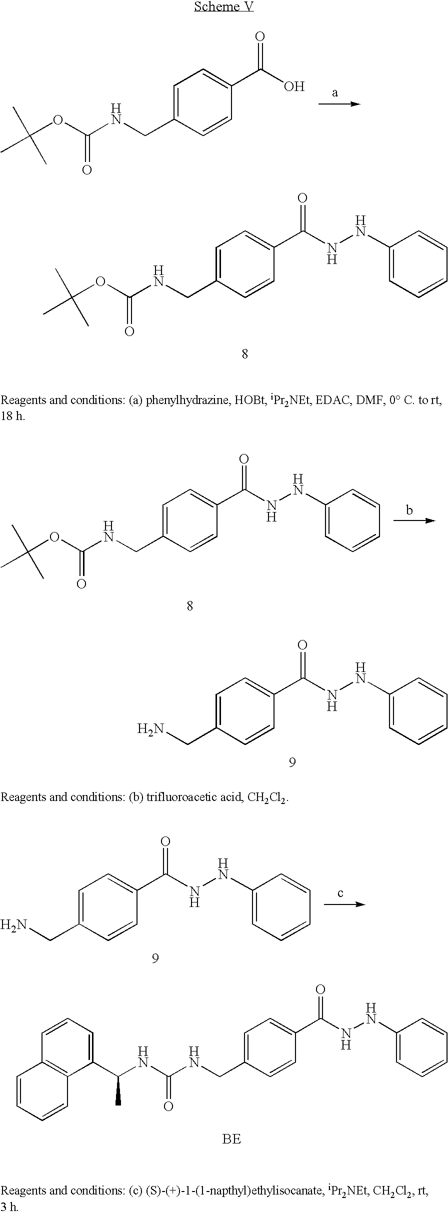

### Example 5

### (S)-4-((3-(1-(naphthalen-1-yl)ethyl)ureido)methyl)-N′-phenylbenzohydrazide (BE)

Preparation of tert-butyl (N′-phenylbenzohydrazide)benzylcarbamate (8): A solution of 4-(Boc-aminomethyl)benzoic acid (2.0 g, 8.0 mmol) in DMF (40 mL) at 0° C. was treated with 1-hydroxybenzotriazole hydrate (1.3 g, 9.6 mmol), N,N-diisopropylethylamine (5.5 mL, 32.0 mmol) and N-(3-dimethylaminopropyl)-N′-ethylcarbodiimide hydrochloride (1.8 g, 9.6 mmol). The mixture was stirred at 0° C. for 1 hour. Phenylhydrazine (0.9 mL, 9.6 mmol) was added, the mixture was warmed to room temperature and stirred for 18 hours. The reaction was quenched by pouring into saturated aqueous NaHCO₃ and extracted three times with EtOAc. The combined organic layers were washed with brine, dried (MgSO₄), filtered and concentrated in vacuo. The crude material was purified over silica (CHCl₃ to 5% methanol/CHCl₃) to afford 2.5 g of 8: ¹H NMR (400 MHz, CDCl₃) δ 10.31 (d, J=2.4 Hz, 1H), 7.95-7.85 (m, 3H), 7.49 (t, J=6.0 Hz, 1H), 7.34 (d, J=8.0 Hz, 2H), 7.14 (dd, J=8.0, 7.2 Hz, 2H), 6.76 (d, J=8.0 Hz, 2H), 6.71 (d, J=7.6 Hz, 1H), 4.18 (d, J=6.0 Hz, 2H), 1.40 (s, 9H); ESI⁻ MS: m/z (rel intensity) 342 (100, [M+H]).

Preparation of 4-(aminomethyl)-N′-phenylbenzohydrazide (9): A solution of tert-butyl (N′-phenylbenzohydrazide)benzylcarbamate, 8, (1.5 g, 4.4 mmol) in CH₂Cl₂ (30 mL) was treated with trifluoroacetic acid (30 mL) and stirred at room temperature for 1 hour. The mixture was concentrated in vacuo to provide the desired product as a trifluoroacetic acid salt, which was used without further purification: ¹H NMR (400 MHz, d₆-DMSO) δ 10.42 (s, 1H), 8.29 (bd s, 2H), 7.95 (d, J=8.0 Hz, 2H), 7.57 (d, J=8.8 Hz, 2H), 7.15 (dd, J=8.8, 7.6 Hz, 2H), 6.78 (d, J=7.6 Hz, 2H), 6.72 (t, J=7.2 Hz, 1H), 4.12 (dd, J=11.6, 5.6 Hz, 2H); ESI⁻ MS: m/z (rel intensity) 242 (100, [M+H]).

Preparation of (S)-4-((3-(1-(naphthalen-1-yl)ethyl)ureido)methyl)-N′-phenylbenzohydrazide (BE): To a solution of 4-(aminomethyl)-N′-phenylbenzohydrazide, 9, (0.26 g, 1.10 mmol) and N,N-diisopropylethylamine (1.17 mL, 6.60 mmol) in CH₂Cl₂ (6 mL) was added (S)-(+)-1-(1-naphthyl)ethylisocanate (0.22 g, 1.10 mmol). The resultant mixture was stirred at room temperature for 3 hours. The reaction was quenched by pouring into a saturated aqueous solution of sodium bicarbonate and extracted with EtOAc. The combined organic layers were dried (MgSO₄), filtered and concentrated in vacuo. The crude material was purified over silica (100% CHCl₃ to 5% methanol/CHCl₃) to afford 140 mg of BE as a tan solid: ¹H NMR (400 MHz, d₆-DMSO) δ 10.31 (d, J=2.4 Hz, 1H), 8.13 (d, J=8.0 Hz, 1H), 7.94 (d, J=7.6 Hz, 1H), 7.90 (m, 1H), 7.85-7.81 (m, 3H), 7.59-7.48 (m, 4H), 7.32 (d, J=8.0 Hz, 2H), 7.15 (t, J=7.2 Hz, 2H), 6.77 (d, J=7.6 Hz, 2H), 6.71 (t, J=7.6 Hz, 1H), 6.66 (d, J=8.4 Hz, 1H), 5.57 (t, J=7.6 Hz, 1H), 4.28 (t, J=5.2 Hz, 2H), 1.48 (d, J=6.8 Hz, 3H); ESI⁺ MS: m/z (rel intensity) 439.2 (100, [M+H]).

### Compounds of Category I

### Third Aspect

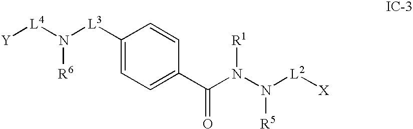

wherein Y-L⁴-N(R⁶)—, L³, R¹, -L²-X, and R⁵ are defined herein below in Table 3.

TABLE 3

| Cmpd. | Y-L⁴-N(R⁶)- | L³ | R¹ | R⁵ | -L²-X |
|---|---|---|---|---|---|
| BF | 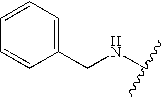 | CH₂ | H | CH₃ | 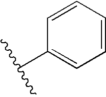 |
| BG | 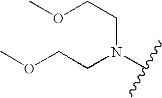 | CH₂ | H | CH₃ | 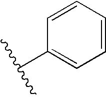 |
| BH | 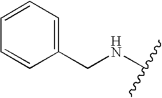 | CH₂ | H | (CH₂)₃NH₂ | 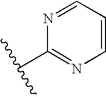 |
| BI | 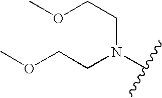 | CH₂ | H | (CH₂)₃NH₂ | 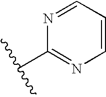 |
| BJ | 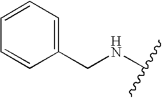 | CH₂ | H | 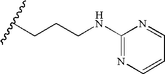 | 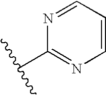 |
| BK | 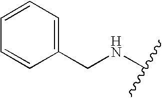 | CH₂ | H | 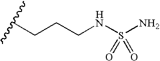 | 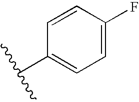 |
| BL | 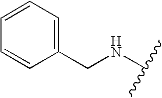 | CO | H | (CH₂)₃NH₂ | 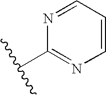 |
| BM | 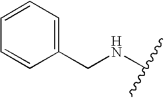 | CO | H | CH₃ | 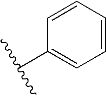 |
| BN | 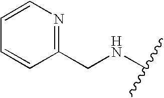 | CO | H | CH₃ | 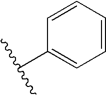 |
| BO | 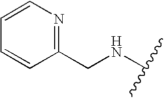 | CO | H | CH₃ | 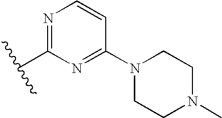 |
| BP | 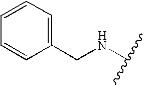 | CH₂ | 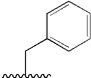 | H | 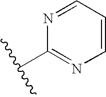 |
| BQ | 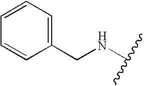 | CH₂ | 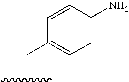 | H | 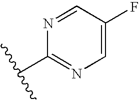 |
| BR | 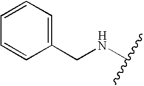 | CH₂ | 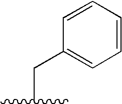 | H | 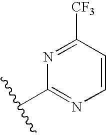 |
| BS | 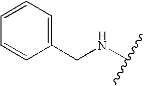 | CH₂ | 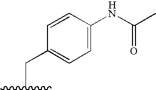 | H | 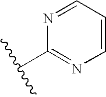 |
| BT | 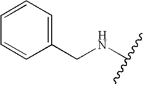 | CH₂ | 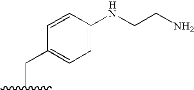 | H | 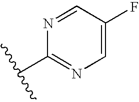 |
| BU | 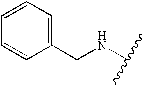 | CH₂ | 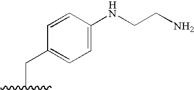 | H | 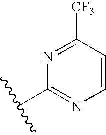 |
| BV | 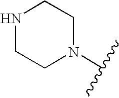 | CO | 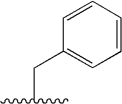 | H | 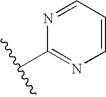 |
| BW | 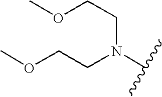 | CH₂ | 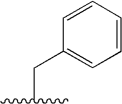 | H | 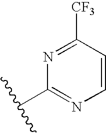 |
| BX | 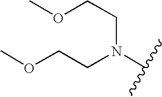 | CO | 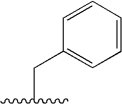 | H | 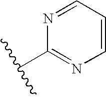 |
| BY | 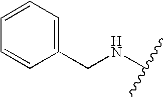 | CH₂ | 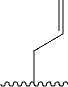 | H | 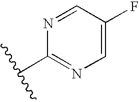 |
| BZ | 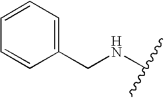 | CH₂ | 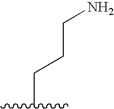 | H | 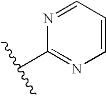 |
| CA | 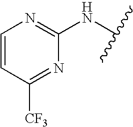 | CH₂ | 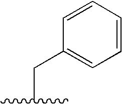 | H | 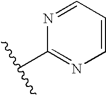 |
| CB | 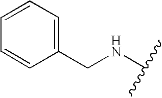 | CH₂ | 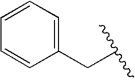 | H | 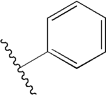 |
| CC | 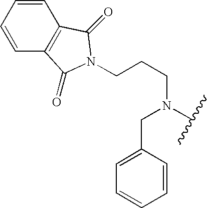 | CH₂ | H | H | 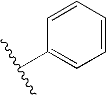 |
| CD | 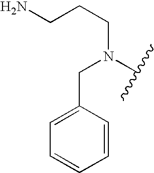 | CH₂ | H | H | 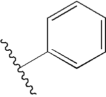 |
| CE | 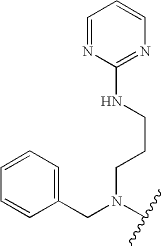 | CH₂ | H | H | 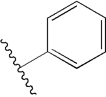 |
| CF | 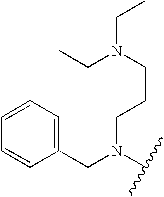 | CH₂ | H | H | 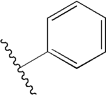 |
| CG | 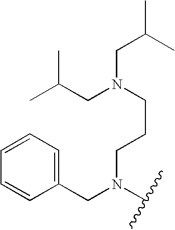 | CH₂ | H | 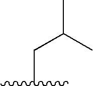 | 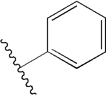 |
| CH | 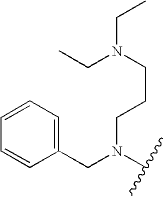 | CH₂ | H | CH₂CH₃ | 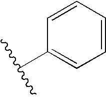 |
| CI | 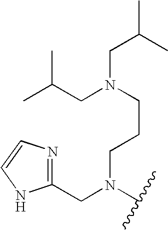 | CH₂ | H | H | 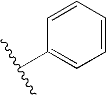 |
| CJ | 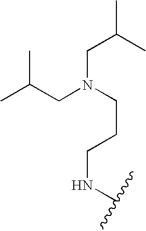 | CH₂ | H | 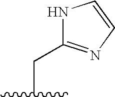 | 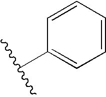 |
| CK | 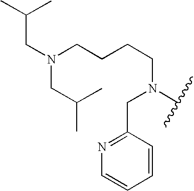 | CH₂ | H | H | 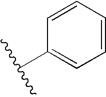 |
| CL | 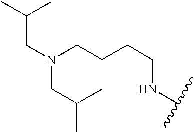 | CH₂ | H | H | 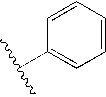 |
| CM | 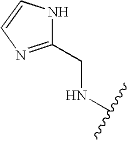 | CH₂ | H | 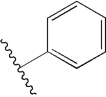 | |
| CN | 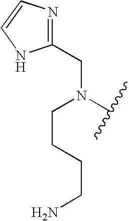 | CH₂ | H | H | 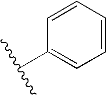 |
| CO | 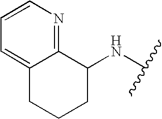 | CH₂ | H | H | 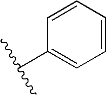 |
| CP | 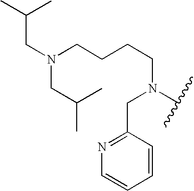 | CH₂ | H | 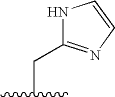 | 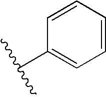 |
| CQ | 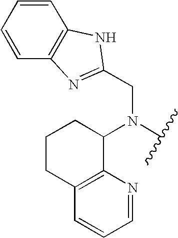 | CH₂ | H | H | 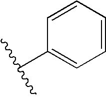 |
| CR | 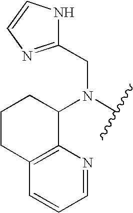 | CH₂ | H | CH₃ | 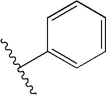 |
| CS | 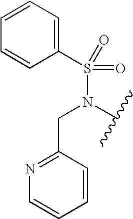 | CH₂ | H | H | 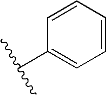 |
| CT | 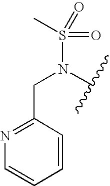 | CH₂ | H | H | 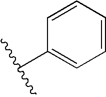 |
| CU | 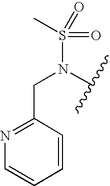 | CH₂ | H | SO₂Me | 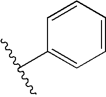 |
| CV | 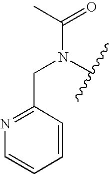 | CH₂ | H | H | 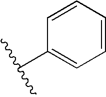 |
| CW | 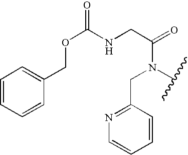 | CH₂ | H | H | 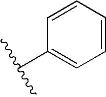 |
| CX | 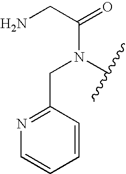 | CH₂ | H | H | 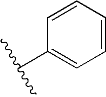 |
| CY | 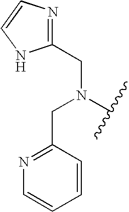 | CH₂ | 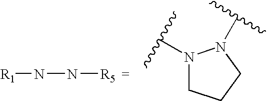 | | 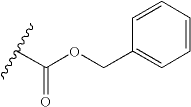 |
| CZ | 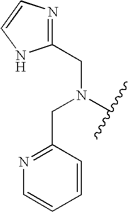 | CH₂ | 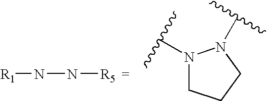 | | H |
| DA | 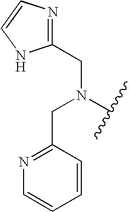 | CH₂ | 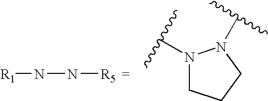 | | 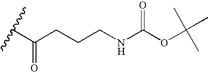 |
| DB | 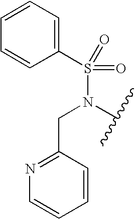 | CH₂ | 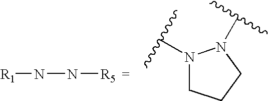 | | H |
| DC | 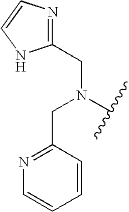 | CH₂ | 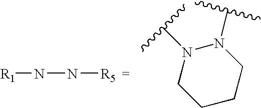 | | 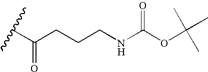 |
| DD | 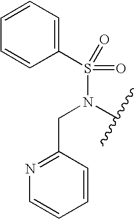 | CH₂ | H | 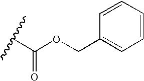 | H |
| DE | 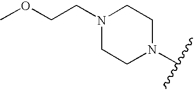 | CH₂ | H | H | 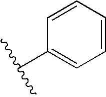 |

The compounds which comprise the third aspect of Category I of the present invention can be prepared by the procedure outlined herein below in Scheme VI.

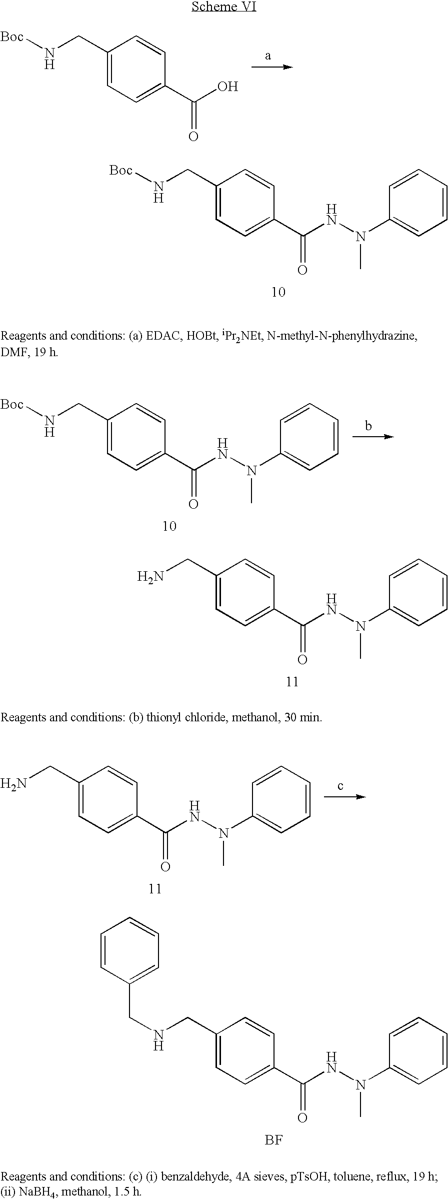

### Example 6

### 4-((benzylamino)methyl)-N′-methyl-N′-phenylbenzohydrazide (BF)

Preparation of N′-methyl-4-((tert-butoxycarbonyl)methyl)-N′-phenylbenzohydrazide (10): To a cold (0° C.) solution of 4-((tert-butoxycarbonyl)methyl)-benzoic acid (3.5 g, 13.9 mmol), 1-hydroxybenzotriazole hydrate (2.3 g, 16.7 mmol) and N,N-diisopropylethylamine (7.3 mL, 41.7 mmol) in DMF (60 mL) was added N-(3-dimethylaminopropyl)-N′-ethylcarbodiimide hydrochloride (3.2 g, 16.7 mmol). After stirring the reaction at 0° C. for 1 hour, N-methyl-N-phenylhydrazine (3.3 mL, 27.8 mmol) was added to the mixture and warmed to room temperature. After stirring for 19 hours, the solution was poured into aqueous saturated NaHCO₃ and extracted with EtOAc. The organic phase was washed with brine, dried (MgSO₄), filtered and concentrated in vacuo. The residue was purified over silica (2% methanol/CHCl₃ to 20% methanol/CHCl₃) to afford 4.5 g of the desired product as a white solid: ¹H NMR (300 MHz, d₆-DMSO) δ 10.60 (s, 1H), 7.84 (d, J=8.0 Hz, 2H), 7.50 (t, J=6.0 Hz, 1H), 7.34 (d, J=8.0 Hz, 2H), 7.20 (dd, J=9.2, 7.2 Hz, 2H), 6.80 (d, J=8.0 Hz, 2H), 6.75 (t, J=7.2 Hz, 1H), 4.18 (d, J=6.0 Hz, 2H), 3.19 (s, 3H), 1.40 (s, 9H).

Preparation of 4-(aminomethyl)-N′-methyl-N′-phenylbenzohydrazide (11): To a solution of N′-methyl-4-((tert-butoxycarbonyl)methyl)-N′-phenylbenzohydrazide, 10, (4.5 g, 12.7 mmol), in methanol (40 mL) was added dropwise thionyl chloride (2 mL). After stirring the reaction for 45 minutes at room temperature, the mixture was concentrated in vacuo. The crude oil was neutralized with aqueous saturated NaHCO₃. The aqueous phase was extracted five times with 20% isopropanol/CHCl₃. The combined organic phases were dried (MgSO₄), filtered and concentrated in vacuo. The resulting residue was used without further purification: ¹H NMR (300 MHz, d₆-DMSO) δ 10.64 (s, 1H), 8.42 (s, 2H), 7.90 (d, J=8.0 Hz, 2H), 7.57 (d, J=8.0 Hz, 2H), 7.18 (dd, J=7.6, 7.6 Hz, 2H), 6.77 (d, J=8.4 Hz, 2H), 6.73 (t, J=7.2 Hz, 1H), 4.07 (ddd, J=11.2, 11.2, 5.2 Hz, 2H), 3.16 (s, 3H).

Preparation of 4-((benzylamino)methyl)-N′-methyl-N′-phenylbenzohydrazide (BF): To a solution of 4-(aminomethyl)-N′-methyl-N′-phenylbenzohydrazide, 11, (0.78 g, 3.05 mmol), benzaldehyde (0.36 g, 3.35 mmol) and 4 angstrom molecular sieves in toluene (30 mL) was added para-toluene sulfonic acid (0.06 g, 0.31 mmol). The reaction flask was fitted with a Dean-Stark trap and the mixture was heated to reflux. After stirring the reaction for 18 hours at reflux, the mixture was poured into aqueous saturated NaHCO₃ and extracted with EtOAc. The organic phase was dried (MgSO₄), filtered and concentrated in vacuo to provide an intermediate imine: ¹H NMR (300 MHz, d₆-DMSO) δ 10.62 (s, 1H), 8.54 (s, 1H), 7.88 (d, J=8.0 Hz, 2H), 7.80 (dd, J=7.2, 2.0 Hz, 2H), 7.48-7.44 (m, 4H), 7.20 (t, J=8.0 Hz, 2H), 6.80 (d, J=8.0 Hz, 2H), 6.75 (t, J=7.2 Hz, 1H), 4.85 (s, 2H), 3.19 (s, 3H),

The crude imine was diluted with methanol (8 mL) and sodium borohydride (0.10 g, 2.50 mmol) was added all at once. After stirring the reaction mixture at room temperature for 1.5 hours, the solution was poured into aqueous saturated NaHCO₃ and extracted with EtOAc. The organic phase was dried (MgSO₄), filtered and concentrated in vacuo. The resulting residue was purified over silica (2% methanol/CHCl₃ to 20% methanol/CHCl₃) to afford 250 mg of BF as a white solid: ¹H NMR (300 MHz, d₆-DMSO) δ 10.61 (s, 1H), 7.85 (d, J=8.4 Hz, 2H), 7.47 (d, J=8.4 Hz, 2H), 7.33-7.28 (m, 4H), 7.24-7.18 (m, 3H), 6.80 (d, J=8.0 Hz, 2H), 6.75 (t, J=7.2 Hz, 1H), 3.74 (s, 2H), 3.69 (s, 2H), 3.19 (s, 3H); ESI⁺ MS: m/z (rel intensity) 346 (100, M+H).

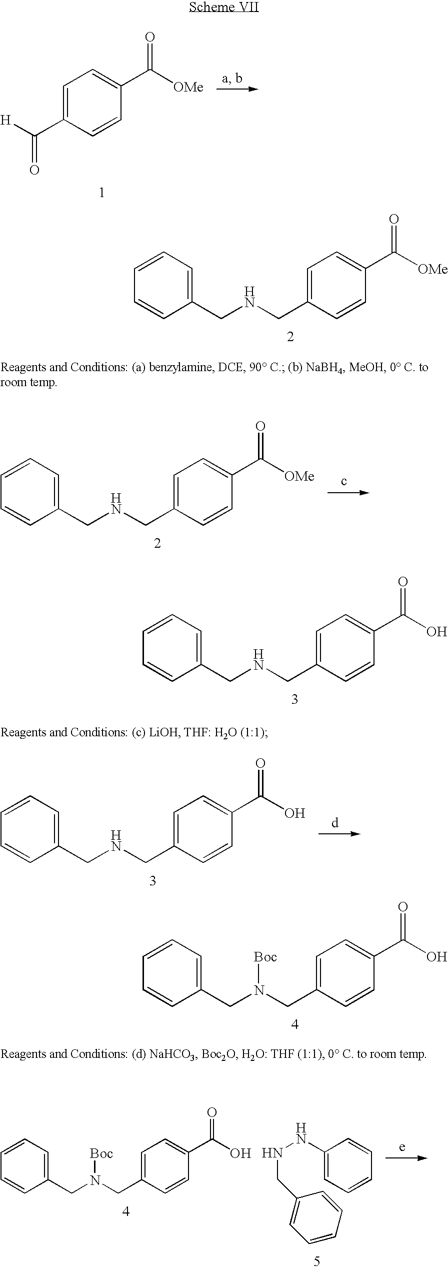

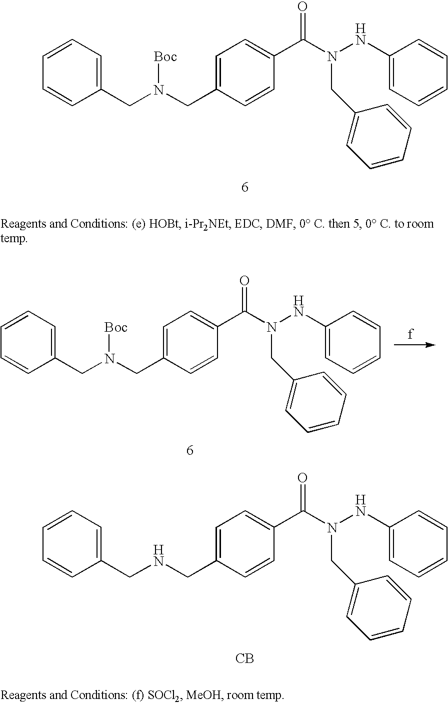

### Example 7

### N-benzyl-4-((benzylamino)methyl)-N′-phenylbenzohydrazide (CB)

Preparation of methyl 4-((benzylamino)methyl)benzoate (2): A solution of 1 (1.00 g, 6.09 mmol) in DCE (10 mL) was treated with benzylamine (800 μL, 7.31 mmol, 1.2 equiv.). The resulting mixture was warmed to 65° C. and stirred for 15 h. The reaction was cooled to room temperature and concentrated. The imine was redissolved in MeOH (10 mL). The mixture was cooled to 0° C. and NaBH₄ (1.11 g, 29.24 mmol, 4.8 equiv.) was added portion-wise. The reaction was then slowly warmed to room temperature and stirred for 4 h. A saturated aqueous solution of sodium bicarbonate (10 mL) was added. The product was extracted three times with 5 mL of CH₂Cl₂. The combined organic layers were washed dried over potassium carbonate, filtered and concentrated. The crude material was purified by chromatography using the ISCO (silica gel, 2-5% MeOH/CHCl₃) to afford 1.55 g (quantitative yield) of pure product: ¹H NMR (400 MHz, CDCl₃) [symbol] 7.96 (d, J=8.4 Hz, 2H), 7.43 (d, J=8.0 Hz, 2H), 7.36-7.18 (m, 5H), 3.88 (s, 3H), 3.58 (s, 2H), 3.55 (s, 2H).

Preparation of 4-((benzylamino)methyl)benzoic acid (3): A solution of 4-((benzylamino)methyl)benzoate, 2, (1.55 g, 6.09 mmol) and lithium hydroxide (0.59 mmol, 24.36 mmol, 4.0 equiv.) in a 1:1 mixture of THF:H₂O was warmed to 70° C. and stirred for 17 h. The reaction was then cooled to room temperature. Ethyl acetate (10 mL) was added. The layers were separated. The aqueous layer was acidified to pH˜4. The aqueous layer was then concentrated. The crude residue was redissolved in MeOH (10 mL) then filtered to remove the salts. The product was concentrated and dried to afford 1.02 g (70% yield) of product: Crude ¹H NMR (400 MHz, d⁶-DMSO) [symbol] 10.03 (bs, 1H), 7.92 (d, J=8.4 Hz, 2H), 7.67 (d, J=8.0 Hz, 2H), 7.57-7.55 (m, 2H), 7.40-7.36 (m, 3H), 4.20-4.07 (m, 4H).

Preparation of 4-((tert-butoxycarbonyl)benzylamino)methyl)benzoic acid (4): To a solution of 4-((benzylamino)methyl)benzoic acid, 3, (0.10 g, 0.41 mmol, 1.0 equiv.) in a 1:1 mixture of H₂O and THF at 0° C. were added sodium bicarbonate (55 mg, 0.62 mmol, 1.5 equiv.) and Boc anhydride (110 mg, 0.49 mmol, 1.2 equiv.). After 30 min at 0° C., the reaction was warmed to room temperature and stirred for 18 h. The layers were then separated; the impurities were extracted with diethyl ether (10 mL). The aqueous layer was acidified with a 1N aqueous solution of HCl. The product was then extracted three times with CH₂Cl₂ (5 mL). The combined organic layers were dried over Na₂SO₄, filtered and concentrated to afford 0.08 g (57% yield) of product: Crude ¹H NMR (400 MHz, CDCl₃) [symbol] 8.04 (d, J=8.4 Hz, 2H), 7.36-7.14 (m, 7H), 4.50-4.30 (m, 4H), 1.49 and 1.45 (bs and bs due to rotomers, 9H); ESI⁺ MS: m/z (rel intensity) 364.2 (100, [M+H]⁺).

Preparation of hydrazide (6): A solution of 4-((tert-butoxycarbonyl)methyl)benzoic acid, 4, (0.11 g, 0.32 mmol, 1.0 equiv.) in DMF (5 mL) at 0° C. was treated with HOBt (0.55 g, 0.38 mmol, 1.2 equiv.), Hünigs base (225 μL, 1.28 mmol, 4.0 equiv.) and EDC (75 mg, 0.38 mmol, 1.2 equiv.). After 1 h at 0° C., hydrazine 5 (130 mg, 0.64 mmol, 2.0 equiv.) was added to the reaction that was slowly warmed to room temperature and stirred for 18 h. A saturated aqueous solution of sodium bicarbonate (10 mL) was added. The product was extracted three times with 10 mL of EtOAc. The combined organic layers were washed with brine, dried over magnesium sulfate, filtered and concentrated. The crude material was purified by chromatography using the ISCO (silica gel, 0-5% MeOH/CHCl₃) to afford 0.16 g (96% yield) of product: ¹H NMR (400 MHz, CDCl₃) [symbol] 8.36 (bs, 1H), 7.64 (d, J=7.6 Hz, 2H), 7.36 (d, J=6.8 Hz, 2H), 7.34-7.12 (m, 10H), 7.91 (d, J=8.4 Hz, 2H), 6.86-6.80 (m, 2H), 4.81 (s, 2H), 4.45-4.26 (m, 4H), 1.45 (bs, 9H); ESI⁺ MS: m/z (rel intensity) 522.3 (80, [M+H]⁺).

Preparation of N-benzyl-4-((benzylamino)methyl)-N′-phenylbenzohydrazide (CB): A solution of hydrazide, 6, (0.16 g, 0.30 mmol, 1.0 equiv.) in MeOH (5 mL) was treated with 1 mL of thionyl chloride. The resulting mixture was stirred at room temperature for 18 h. The solvents were then removed by evaporation. The resulting oil was stirred and triturated in Et₂O. The solid was filtered and washed with EtOAc then dried to afford 0.08 g (62% yield) of product: ¹H NMR (400 MHz, d⁶-DMSO, peaks are very broad) [symbol] 7.75-7.60 (m, 2H), 7.52-6.86 (m, 15H), 6.72-6.50 (m, 2H), 4.60 (bs, 2H), 4.00-3.75 (m, 4H); ESI⁺ MS: m/z (rel intensity) 422.2 (95, [M+H]⁺).

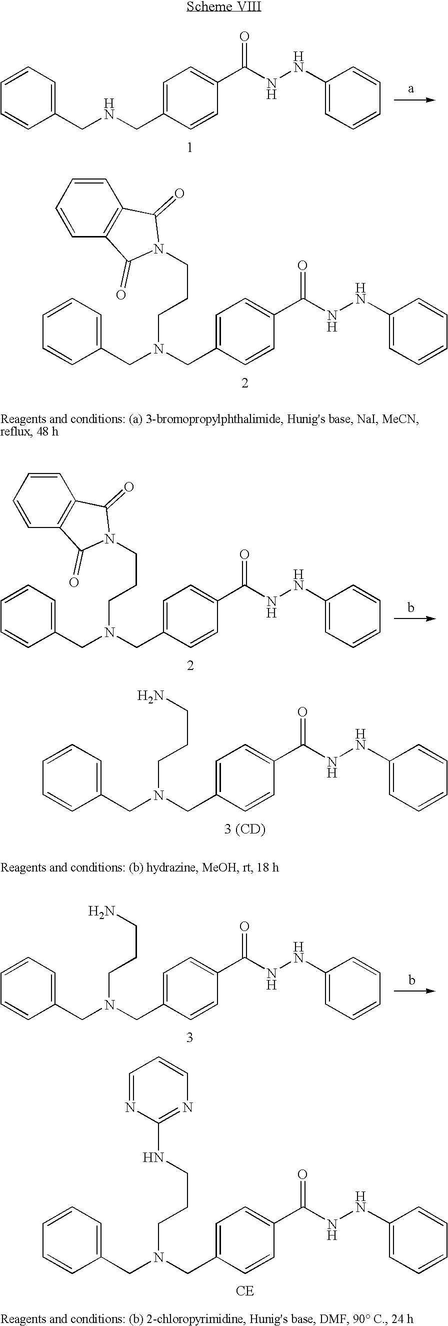

### Example 8

### 4-((benzyl(3-(pyrimidin-2-ylamino)propyl)amino)methyl)-N′-phenyl-benzohydrazide (CE)

Preparation of 4-((benzyl(3-(1,3-dioxoisoindolin-2-yl)propyl)amino)methyl)-N′-phenylbenzohydrazide (2): To a solution of 4-((benzylamino)methyl)-N′-phenylbenzohydrazide, 1, (0.20 g, 0.60 mmol) in acetonitrile (20 mL) was added 3-bromopropylphthalimide (0.49 g, 1.80 mmol), Hunig's base (0.84 mL, 4.80 mmol), and sodium iodode (0.01 g, 0.06 mmol). The reaction was heated to reflux and stirred for 48 h. The reaction was diluted into EtOAc (40 mL) and washed with saturated aqueous NaHCO₃. The organics were collected and dried over MgSO₄, filtered, and concentrated in vacuo. The crude residue was purified by silica gel chromatography (0-15% MeOH/CHCl₃) to afford 300 mg 2 as orange viscous oil: ¹H NMR (400 MHz, CDCl₃) δ 7.79, (dd, J=5.2, 3.2 Hz, 2H), 7.73 (d, J=8.0 Hz, 2H), 7.67 (dd, J=5.6, 3.2 Hz, 2H), 7.46 (d, J=8.0 Hz, 2H), 7.34-7.17 (m, 7H), 6.95-6.88 (m, 3H), 3.66 (m, 2H), 3.59 (s, 2H), 3.55 (s, 2H), 2.48 (m, 2H), 1.86 (m, 2H); ESI⁺ MS: m/z (rel intensity) 519 (100, M+H).

Preparation of 4-(((3-aminopropyl)(benzyl)amino)methyl)-N′-phenyl-benzohydrazide (3) (CD): To a solution of 4-((benzyl(3-(1,3-dioxoisoindolin-2-yl)propyl)amino)methyl)-N′-phenylbenzohydrazide, 2, (0.43 g, 0.83 mmol) in MeOH (10 mL) was added hydrazine (0.08 mL, 2.49 mmol). The reaction was stirred at rt for 18 h. The crude reaction was adsorbed onto Celite®. The crude residue was purified by silica gel chromatography (0-10% MeOH/CHCl₃) to afford 190 mg of 3: ¹H NMR (400 MHz, CDCl₃) δ 7.76 (d, J=8.0 Hz, 2H), 7.44-7.10 (m, 10H), 6.84 (d, J=8.0 Hz, 2H), 3.51 (s, 2H), 3.49 (s, 2H), 2.64, (t, J=6.40 Hz, 2H), 2.40, (t, J=6.40 Hz, 2H), 1.61-1.57 (m, 2H).

Preparation of 4-((benzyl(3-(pyrimidin-2-ylamino)propyl)amino)methyl)-N′-phenylbenzohydrazide (CE): To a solution of 4-(((3-aminopropyl)(benzyl)amino)-methyl)-1′-phenylbenzohydrazide, 3, (0.11 g, 0.28 mmol) in DMF (5 mL) was added 2-chloropyrimidine (0.04 g, 0.31 mmol) and Hunig's base (0.30 mL, 1.70 mmol). The reaction was heated to 90° C. and stirred for 24 h. The crude reaction was concentrated and purified by silica gel chromatography (0-10% MeOH/CHCl₃), yielding 25 mg of CE: ¹H NMR (400 MHz, CDCl₃) δ 8.18 (d, J=4.40 Hz, 2H), 7.74 (d, J=8.0 Hz, 2H), 7.45 (d, J=8.0 Hz, 2H), 7.40-7.18 (m, 7H), 6.94-6.75 (m, 3H), 6.43 (t, J=4.80 Hz, 1H), 3.59 (s, 2H), 3.54 (s, 2H), 3.39 (q, J=6.40 Hz, 2H), 2.46 (t, J=6.40 Hz, 2H), 1.73-1.69 (m, 2H).

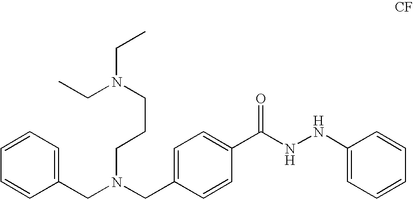

4-((Benzyl(3-(diethylamino)propyl)amino)methyl)-N′-phenylbenzohydrazide (CF): ¹H NMR (400 MHz, d6-DMSO) δ 10.29 (d, 1H), 7.88-7.83 (m, 3H), 7.45 (d, J=7.60 Hz, 2H), 7.34-7.20 (m, 4H), 7.10 (t, J=7.60 Hz, 2H), 6.73 (d, J=8.40 Hz, 2H), 6.67 (t, J=7.20 Hz, 1H), 3.61 (s, 2H), 3.30 (s, 2H), 2.60-2.20 (m, 8H), 0.95-0.75 (m, 6H). ESI⁺ MS: m/z (rel intensity) 431 (100, M+H).

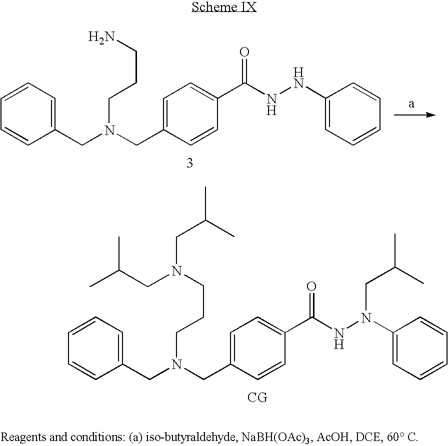

### Example 9

Preparation of 4-((benzyl(3-(diisobutylamino)propyl)amino)methyl)-N′-isobutyl-N′-phenylbenzohydrazide (CG): To a solution of 4-(((3-aminopropyl)(benzyl)-amino)methyl)-N′-phenylbenzohydrazide, 3, (0.09 g, 0.24 mmol) in DCE (3 mL) was added iso-butyraldehyde (0.05 g, 0.73 mmol). The reaction was heated to 60° C. and stirred for 5 min. NaBH(OAc)₃ (0.17 g, 0.81 mmol) and AcOH (0.02 mL), were added and the reaction stirred for 1 h. The crude reaction was diluted into DCM and washed with saturated aqueous NaHCO₃. The organics were collected and dried over MgSO₄, filtered, and concentrated in vacuo. Purification by silica gel chromatography (0-10% MeOH/CHCl₃) yielded 61 mg (45%) of 6: ¹H NMR (400 MHz, CDCl₃) δ 7.81-7.53, (m, 3H), 7.45 (d, J=8.0 Hz, 2H), 7.35-7.18 (m, 6H), 6.88-6.78 (m, 3H), 3.58 (s, 2H), 3.55 (s, 2H), 2.50-2.40 (m, 2H), 2.30-2.22 (m, 2H), 1.97 (d, J=6.80 Hz, 4H), 1.61-1.57 (m, 5H), 1.00 (d, J=6.8 Hz, 6H), 0.79 (d, J=6.40 Hz, 12H); ESI⁺ MS: m/z (rel intensity) 557 (100, M+H).

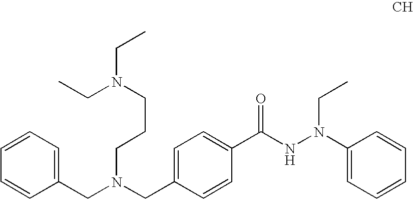

4-((benzyl(3-(diethylamino)propyl)amino)methyl)-N′-ethyl-N′-phenylbenzohydrazide (CH) ¹H NMR (400 MHz, CDCl₃) δ 7.79, (d, J=8.40 Hz, 2H), 7.43 (d, J=8.0 Hz, 2H), 7.32-7.20 (m, 7H), 6.89 (d, J=7.60 Hz, 2H), 6.82 (m, 1H), 3.65 (q, J=7.20 Hz, 2H), 3.57 (s, 2H), 3.56 (s, 2H), 2.65 (q, J=7.20 Hz, 4H), 2.58-2.50 (m, 2H), 2.43 (t, J=6.80 Hz, 2H), 1.72-1.68 (m, 2H), 1.28-1.20 (m, 9H) ESI⁺ MS: m/z (rel intensity) 473 (100, M+H).

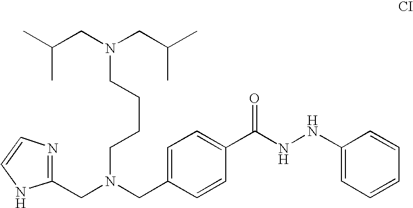

4-((((1H-imidazol-2-yl)methyl)(4-(diisobutylamino)butyl)amino)methyl)-N′-phenylbenzohydrazide (CI): ¹H NMR (400 MHz, DMSO-d6) δ 10.31, (s, 1H), 7.87-7.82 (m, 3H), 7.52 (d, J=8.00 Hz, 2H), 7.29 (s, 1H), 7.18-7.07 (m, 3H), 6.73 (d, J=8.40 Hz, 2H), 6.67 (m, 1H), 3.93 (s, 2H), 3.65 (s, 2H), 2.38 (m, 2H), 2.13 (m, 2H), 1.92 (m, 4H), 1.52 (m, 2H), 1.40 (m, 2H), 1.22 (m, 2H), 0.75 (d, J=6.00 Hz, 12H); ESI⁺ MS: m/z (rel intensity) 505 (100, M+H).

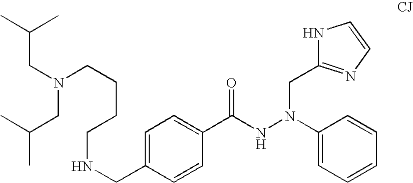

N′-((1H-imidazol-2-yl)methyl)-4-((4-(diisobutylamino)butylamino)methyl)-N′-phenylbenzohydrazide (CJ): ¹H NMR (400 MHz, DMSO-d6) δ 10.29 (s, 1H), 7.52 (d, J=8.00 Hz, 2H), 7.87-7.81 (m, 3H), 7.44 (d, J=8.0a0 Hz, 2H), 7.02 (bs, 0.5H), 6.78 (bs, 0.5H), 7.10 (d, J=7.60 Hz, 2H), 6.73 (d, J=8.40 Hz, 2H), 6.70-6.65 (m, 1H), 3.57 (s, 4H), 2.30 (m, 2H), 2.14 (m, 2H), 1.92 (d, J=6.80 Hz, 4H), 1.74-1.68 (m, 2H), 1.57-1.49 (m, 2H), 1.48-1.37 (m, 2H), 1.29-1.21 (m, 2H), 0.76 (d, J=6.00 Hz, 12H). ESI⁺ MS: m/z (rel intensity) 505 (100, M+H).

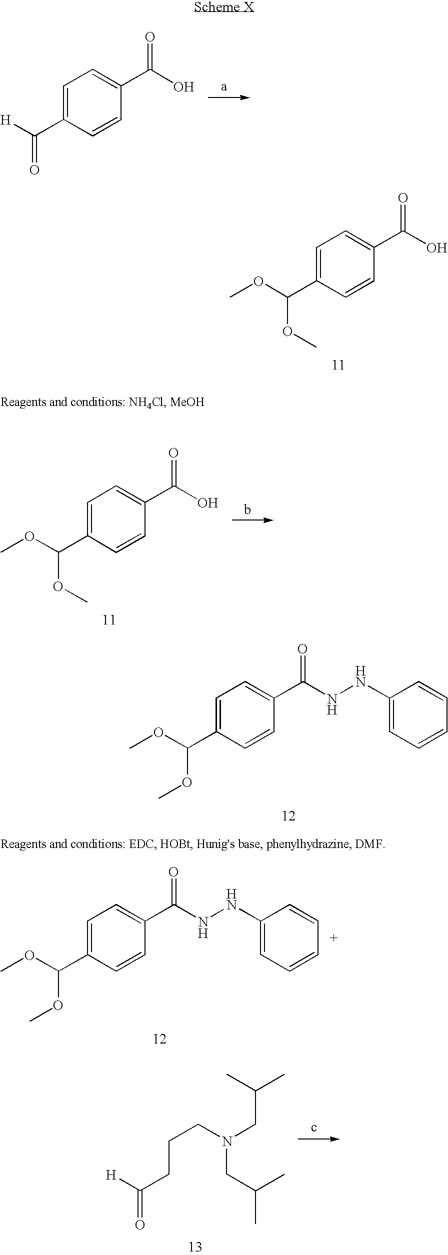

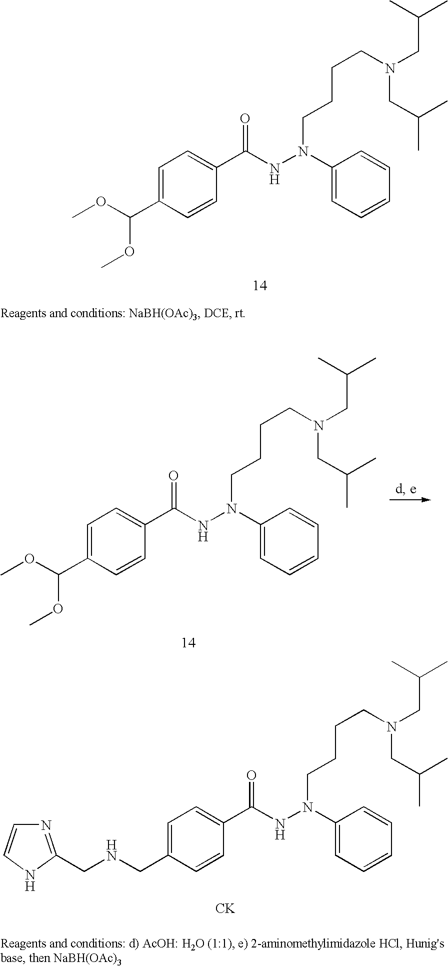

### Example 10

### 4-(((1H-imidazol-2-yl)methylamino)methyl)-N′-(4-(diisobutylamino)butyl)-N′-phenylbenzohydrazide (CK)

Preparation of 4-(dimethoxymethyl)benzoic acid (11): To a solution of 4-formylbenzoic acid (4 g, 27 mmol) in methanol (80 mL) was added NH₄Cl (8 g, 150 mmol). The reaction was heated to reflux and stirred for 18 h. The reaction was concentrated in vacuo. The crude material was dissolved in hot hexanes and filtered to remove NH₄Cl. The product was crystallized from hexanes yielding 2.7 g (51%) of 11 as a white crystalline solid: ¹H NMR (400 MHz, DMSO-d6) δ 13.00 (bs, 1H), 7.91 (d, J=8.40 Hz, 2H), 7.44 (d, J=8.40 Hz, 2H), 5.41 (s, 1H), 3.22 (s, 6H).

Preparation of 4-(dimethoxymethyl)-N′-phenylbenzohydrazide (12): To a 0° C. solution of 4-(dimethoxymethyl)benzoic acid, 11, (2.73 g, 13.90 mmol) in DMF (250 mL) was added HOBt (2.26 g, 16.70 mmol), Hunig's base (7.19 g, 9.70 mL, 27.83 mmol), and EDC (3.20 g, 16.70 mmol). The reaction was stirred 1 h, then phenylhydrazine (3.00 g, 27.83 mmol) was added. The reaction was stirred for 24 h while warming to rt. The reaction was diluted into EtOAc and was washed with saturated aqueous NaHCO₃. The organics were dried over MgSO₄, filtered, and concentrated in vacuo. The crude product 12 was used in the next reaction without further purification. ESI⁺ MS: m/z (rel intensity) 595 (100, 2M+Na).

Preparation of N′-(4-(diisobutylamino)butyl)-4-(dimethoxymethyl)-N′-phenylbenzohydrazide (14): To a solution of 4-(dimethoxymethyl)-N′-phenylbenzohydrazide, 12, (0.92 g, 3.20 mmol) in DCE (30 mL) was added amine 13 (0.70 g, 3.50 mmol) and NaBH(OAc)₃ (1.35 g, 6.40 mmol). The reaction was stirred at rt for 20 h. The reaction was diluted into DCM (30 mL) and was washed with saturated aqueous NaHCO₃ (40 mL). The organics were dried over MgSO₄, filtered, and concentrated in vacuo. The crude residue was purified by silica gel chromatography (0-5% MeOH/DCM) yielded 420 mg (28%) of 14: ¹H NMR (400 MHz, CDCl₃) δ 13.00 (bs, 1H), 7.83 (d, J=8.40 Hz, 2H), 7.55 (d, J=8.40 Hz, 2H), 7.25-7.20 (m, 3H), 6.89-6.80 (m, 3H), 5.43 (s, 1H), 3.60-3.56 (m, 2H), 2.31 (d, J=7.20 Hz, 2H), 2.00 (d, J=7.20 Hz, 4H), 1.75-1.55 (m, 4H), 1.52-1.43 (m, 2H), 0.81 (d, J=6.80 Hz, 12H); ESI⁺ MS: m/z (rel intensity) 470 (100, M+H).

Preparation of 4-(((1H-imidazol-2-yl)methylamino)methyl)-N′-(4-(diisobutylamino)butyl)-N′-phenylbenzohydrazide (CK): To a round bottom flask containing N′-(4-(diisobutylamino)butyl)-4-(dimethoxymethyl)-N′-phenylbenzohydrazide, 14, (0.42 g, 0.90 mmol) was added 1:1 AcOH/H₂O (10 mL). The reaction was stirred at rt for 1.5 h. The reaction was diluted into EtOAc and washed with aqueous saturated NaHCO₃. The organics were dried over MgSO₄, filtered, and concentrated in vacuo. The crude material was added to a flask containing aminomethyl imidazole hydrochloride salt (0.48 g, 0.28 mmol) and Hunig's base (0.07 g, 0.59 mmol). The reaction stirred for 5 minutes, then NaBH(OAc)₃ (0.13 g, 0.59 mmol) was added and the reaction was stirred at rt for 24 h. The crude reaction was diluted into EtOAc and washed with saturated aqueous NaHCO₃. The organics were collected and dried over MgSO₄, filtered, and concentrated in vacuo. The crude residue was purified by silica gel chromatography (0-10% MeOH/CHCl₃) to afford 30 mg (25%) of CK: ¹H NMR (400 MHz, DMSO-d6) δ 10.39 (s, 1H), 7.83 (d, J=8.00 Hz, 2H), 7.46 (d, J=8.00 Hz, 2H), 7.27 (s, 1H), 7.14-7.11 (m, 3H), 6.75 (d, J=8.00 Hz, 2H), 6.70-6.64 (m, 1H), 3.99 (s, 2H), 3.75 (s, 2H), 3.50-3.45 (m, 2H), 2.32-2.23 (m, 2H), 2.04-1.96 (m, 4H), 1.66-1.53 (m, 4H), 1.49-1.40 (m, 2H), 0.76 (d, J=6.80 Hz, 12H); ESI⁺ MS: m/z (rel intensity) 505 (100, M+H).

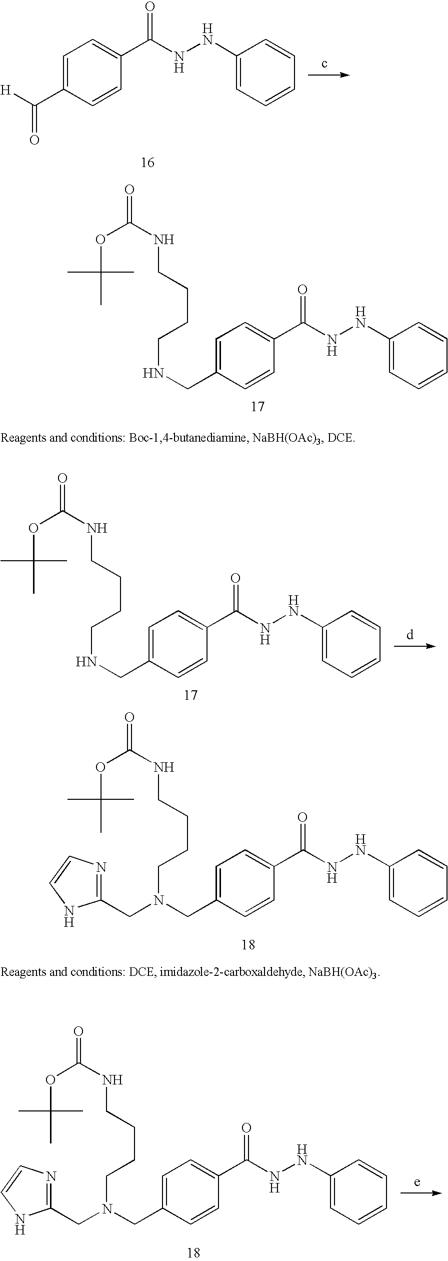

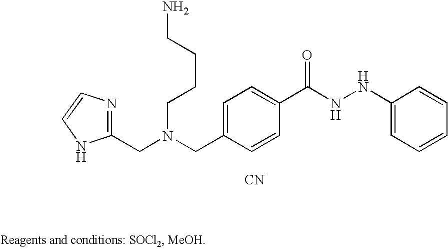

### Example 11

### 4-((((1H-imidazol-2-yl)methyl)(4-aminobutyl)amino)methyl)-N′-phenylbenzohydrazide (CN)

Preparation of (17): To a solution of 4-formyl-N′-phenylbenzohydrazide, 16, (0.25 g, 1.00 mmol) in 1,2-dichloroethane (20 mL) was added Boc-1,4-butanediamine (0.22 g, 1.14 mmol). The reaction was stirred for 10 min at rt, then NaBH(OAc)₃ (0.66 g, 3.12 mmol) was added. The reaction was stirred for 1 h at rt. The reaction was then diluted into EtOAc and washed with aqueous saturated NaHCO₃. The organics were collected and dried over MgSO₄, filtered, and concentrated in vacuo. The crude material was purified by silica gel chromatography (0-20% MeOH/DCM) yielding 300 mg (70%) of 17: ¹H NMR (400 MHz, CDCl₃) δ 7.78 (d, J=8.00 Hz, 2H), 7.42 (d, J=8.00 Hz, 2H), 7.22-7.12 (m, 2H), 6.90-6.76 (m, 3H), 4.72 (bs, 1H), 3.86 (s, 2H), 3.00 (bs, 2H), 2.70-2.62 (m, 2H), 1.90-1.86 (m, 2H), 1.55-1.30 (m, 11H). ESI⁺ MS: m/z (rel intensity) 413 (100, M+H).

Preparation of 18: To a solution of 17 (0.30 g, 0.73 mmol) in 1,2-dichloroethane (20 mL) was added imidazole carboxaldehyde (69 mg, 0.73 mmol) at rt. The reaction was stirred 10 min, then NaBH(OAc)₃ (0.39 g, 1.82 mmol) was added. The reaction stirred at rt for 24 h. The reaction was then diluted into EtOAc and washed with aqueous saturated NaHCO₃. The organics were collected and dried over MgSO₄, filtered, concentrated in vacuo yielding 244 mg (68%) of 18: ¹H NMR (400 MHz, DMSO-d6) δ 10.39 (s, 1H), 7.90-7.80 (m, 3H), 7.45 (d, J=7.60 Hz, 2H), 7.10 (d, J=7.60 Hz, 2H), 6.80-6.65 (m, 4H), 3.59-3.52 (m, 4H), 2.87-2.75 (m, 2H), 2.34-2.22 (m, 2H), 1.45-1.20 (m, 13H); ESI⁺ MS: m/z (rel intensity) 493 (100, M+H).

Preparation of 4-((((1H-imidazol-2-yl)methyl)(4-aminobutyl)amino)methyl)-N′-phenylbenzohydrazide (CN): To a MeOH (3 mL) solution 18 (0.08 g, 0.16 mmol) was added SOCl₂ (3 drops). The reaction was stirred 4 h at rt. The reaction was then diluted into EtOAc and washed with aqueous saturated NaHCO₃. The organics were collected and dried over MgSO₄, filtered, concentrated in vacuo, yielding 58 mg (96%) of CN: ESI⁺ MS: m/z (rel intensity) 393 (100, M+H).

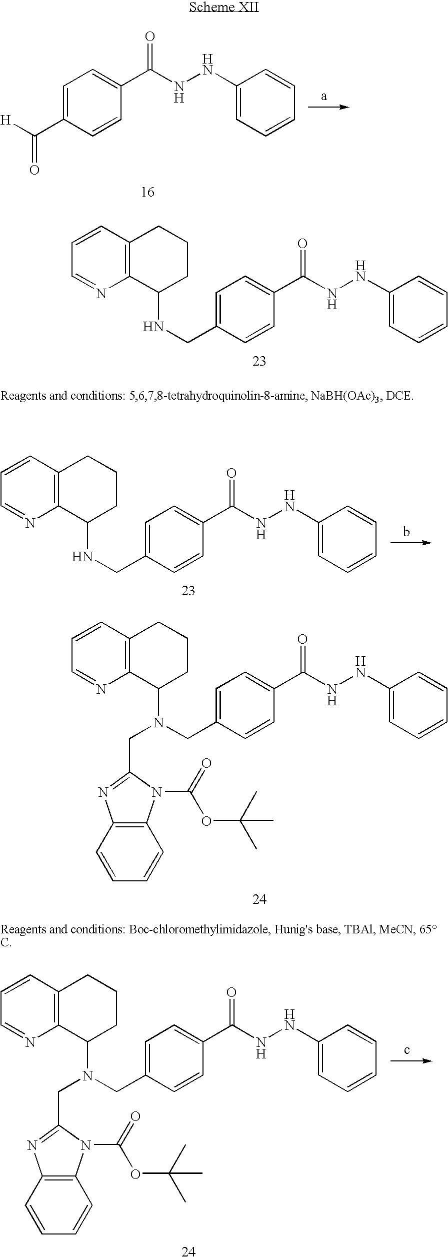

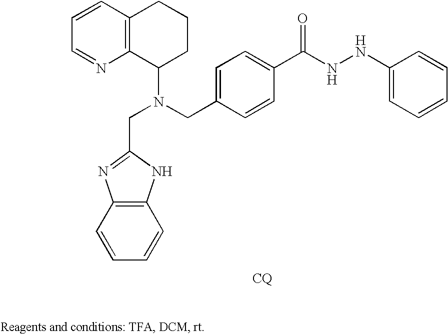

### Example 12

### 4-((((1H-benzo[d]imidazol-2-yl)methyl)(5,6,7,8-tetrahydroquinolin-8-yl)amino)methyl)-N′-phenylbenzohydrazide (CQ)

Preparation of N′-phenyl-4-((5,6,7,8-tetrahydroquinolin-8-ylamino)methyl)benzohydrazide (23) (CO): To a solution of 16 (0.76 g, 3.16 mmol) in 1,2-dichloroethane (20 mL) was added 5,6,7,8-tetrahydroquinolin-8-amine (0.47 g, 3.16 mmol). The reaction was stirred for 10 min at rt, then NaBH(OAc)₃ (1.34 g, 6.32 mmol) was added. The reaction was stirred for 16 h at rt. The reaction was then diluted into EtOAc and washed with aqueous saturated NaHCO₃. The organics were collected and dried over MgSO₄, filtered, and concentrated in vacuo. The crude material was purified by silica gel chromatography (0-10% MeOH/DCM) yielding 800 mg (68%) of 23: ¹H NMR (400 MHz, CDCl₃) δ 8.66 (s, 1H), 8.38 (d, J=3.60 Hz, 1H), 7.71 (d, J=8.00 Hz, 2H), 7.41 (d, J=8.00 Hz, 2H), 7.35 (d, J=6.80 Hz, 1H), 7.22-7.13 (m, 2H), 7.06 (dd, J=7.60, 4.80 Hz, 1H), 6.87-6.79 (m, 3H), 3.97 (d, J=13.60 Hz, 1H), 3.89 (d, J=13.60 Hz, 2H), 3.85-3.78 (m, 1H), 2.75-2.65 (m, 2H), 2.19-2.12 (m, 1H), 2.02-1.93 (m, 1H), 1.81-1.65 (m, 2H).

Preparation of Boc-protected-4-((((1H-benzo[d]imidazol-2-yl)methyl)(5,6,7,8-tetrahydroquinolin-8-yl)amino)methyl)-N′-phenylbenzohydrazide (24): To a solution of N′-phenyl-4-((5,6,7,8-tetrahydroquinolin-8-ylamino)methyl)benzohydrazide, 23, (0.80 g, 2.15 mmol) in acetonitrile (30 mL) was added Hunig's base (0.70 g, 0.93 mL, 5.37 mmol), TBAI (0.16 g, 0.43 mmol), and Boc-chloromethylimidazole (0.57 g, 2.15 mmol). The reaction was heated to 65° C. and stirred 24 h. The reaction was diluted into EtOAc and the organics were washed with aqueous saturated NaHCO₃. The organics were dried over MgSO₄, filtered, and concentrated in vacuo. The crude residue was purified by silica gel chromatography (0-10% MeOH/DCM) to afford 1.1 g (85%) of 24: ¹H NMR (400 MHz, CDCl₃) δ 8.45 (d, J=4.40 Hz, 2H), 7.59 (d, J=7.60 Hz, 2H), 7.51 (d, J=6.80 Hz, 2H), 7.42-7.38 (m, 1H), 7.34-7.10 (m, 8H), 7.02 (dd, J=7.60, 4.80 Hz, 2H), 6.92-6.80 (m, 3H), 6.19 (s, 1H), 4.79 (d, J=14.00 Hz, 1H), 4.66 (d, J=14.00 Hz, 1H), 4.31 (dd, J=10.40, 6.40 Hz, 1H), 3.88 (d, J=14.80 Hz, 1H), 3.75 (d, J=14.80 Hz, 1H), 2.82-2.60 (m, 2H), 2.37-2.25 (m, 1H), 2.08-1.60 (m, 2H). 1.73 (s, 10H).

Preparation of 4-((((1H-benzo[d]imidazol-2-yl)methyl)(5,6,7,8-tetrahydroquinolin-8-yl)amino)methyl)-N′-phenylbenzohydrazide (CQ): To a solution of Boc-protected-4-((((1H-benzo[d]imidazol-2-yl)methyl)(5,6,7,8-tetrahydroquinolin-8-yl)amino)methyl)-N′-phenylbenzohydrazide, 24, (0.08 g, 0.12 mmol) in CH₂Cl₂ was added TFA (2 mL). The reaction stirred at rt for 2 h. The reaction was concentrated under reduced pressure and then diluted into EtOAc. The organics were washed with aqueous saturated NaHCO₃. The organics were dried over MgSO₄, filtered, and concentrated in vacuo to yield 35 mg (60%) of CQ which was used without further purification: ¹H NMR (400 MHz, DMSO-d6) δ 10.24 (s, 1H), 8.57 (d, J=4.80 Hz, 2H), 7.84 (s, 1H), 7.78 (m, 3H), 7.56-7.40 (m, 5H), 7.20 (dd, J=12.40, 4.80 Hz, 2H), 7.13-7.03 (m, 4H), 6.72-6.60 (m, 3H), 4.11-3.75 (m, 5H), 2.84-2.74 (m, 1H), 2.68-2.60 (m, 1H), 2.42-2.34 (m, 1H), 2.00-1.76 (m, 1H), 1.62-1.49 (m, 1H), 1.15-1.07 (m, 1H).

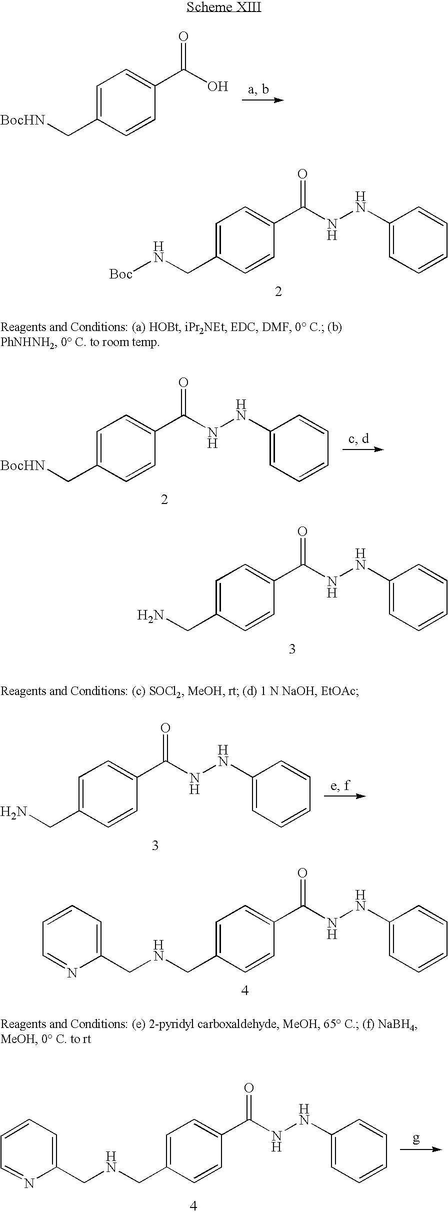

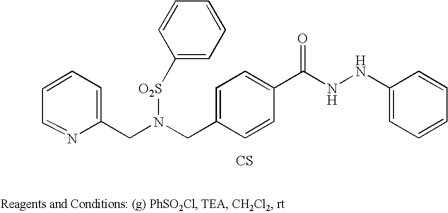

### Example 13

### N-benzyl-4-((benzylamino)methyl)-N′-phenylbenzohydrazide (CS)

Preparation of 4-((tert-butoxycarbonyl)aminomethyl)-N′-phenylbenzohydrazide (2): A solution of 4-((tert-butoxycarbonyl)methyl)benzoic acid (3.00 g, 11.93 mmol) in DMF (15 mL) at 0° C. was treated with HOBt (1.94 g, 14.32 mmol, 1.2 equiv.), Hünigs base (8.32 mL, 47.75 mmol, 4.0 equiv.) and EDC (2.75 g, 14.32 mmol, 1.2 equiv.). The resultant mixture was stirred at 0° C. After 1 h, phenyl hydrazine (2.35 mL, 23.87 mmol, 2.0 equiv.) were added to the reaction that was slowly warmed to room temperature and stirred for 64 h. A saturated aqueous solution of sodium bicarbonate (20 mL) was added. The product was extracted three times with 20 mL of EtOAc. The combined organic layers were washed with brine, dried over magnesium sulfate, filtered and concentrated. The crude material was purified by chromatography using the ISCO (silica gel, 0-10% MeOH/CHCl₃) to afford 3.09 g (76% yield) of pure product: ¹H NMR (400 MHz, CDCl₃) [symbol] 7.95 (bs, 1H), 7.79 (d, J=8.0 Hz, 2H), 7.36 (d, J=8.0 Hz, 2H), 7.25-7.21 (m, 2H), 6.93-6.88 (m, 3H), 6.31 (d, J=4.0 Hz, 1H), 4.33 (d, J=5.6 Hz, 2H), 1.44 (s, 9H); ESI⁺ MS: m/z (rel intensity) 342.1 (100, [M+H]⁺).

Preparation of hydrazide 4-(aminomethyl)-N′-phenylbenzohydrazide (3): A solution of 4-((tert-butoxycarbonyl)aminomethyl)-N′-phenylbenzo-hydrazide, 2, (2.00 g, 5.85 mmol) in MeOH (10 mL) was treated with a large excess of thionyl chloride at room temperature. The resultant mixture was stirred for 18 h. The solvents were then removed by evaporation. The hydrochloride salt was suspended in EtOAc and an aqueous 1N solution of sodium hydroxide was added. The organic layer was separated and dried over magnesium sulfate, filtered and concentrated to afford the free amine. The crude material was purified by chromatography using the ISCO (silica gel, 0-20% MeOH/CH₂Cl₂) to afford 1.10 g (78% yield) of pure product: ¹H NMR (400 MHz, CDCl₃) [symbol] 7.78 (d, J=8.0 Hz, 2H), 7.37 (d, J=8.0 Hz, 2H), 7.25-7.19 (m, 2H), 6.92-6.88 (m, 3H), 3.92 (s, 2H); ESI⁺ MS: m/z (rel intensity) 242.1 (100, [M+H]⁺).

Preparation of N′-phenyl-4-((pyridin-2-ylmethylamino)methyl)-benzohydrazide (4): A solution of 4-(aminomethyl)-N′-phenylbenzohydrazide, 3, (1.10 g, 4.55 mmol, 1.0 equiv.) in MeOH (10 mL) was treated with 2-pyridylcarboxaldehyde (460 μL, 4.78 mmol, 1.05 equiv.). The resulting mixture was warmed to 65° C. and stirred for 2 h. The reaction was then cooled to 0° C. and sodium borohydride (365 mg, 9.57 mmol, 2.1 equiv.) was added. The reaction was then warmed to room temperature and stirred for another 2 h. A saturated aqueous solution of sodium bicarbonate (15 mL) was added. The product was extracted three times with 10 mL of CH₂Cl₂. The combined organic layers were dried over potassium carbonate, filtered and concentrated. The crude material was purified by chromatography using the ISCO (silica gel, 0-10% MeOH/CH₂Cl₂) to afford 0.65 g (43% yield) of pure product: ¹H NMR (400 MHz, CDCl₃) [symbol] 9.02 (bs, 1H), 8.50 (d, J=4.8 Hz, 1H), 7.73 (d, J=8.4 Hz, 2H), 7.61 (dt, J=7.6 Hz, J=2.0 Hz, 1H), 7.32 (d, J=8.0 Hz, 2H), 7.24 (d, J=8.0 Hz, 1H), 7.17-7.11 (m, 3H), 6.85-6.81 (m, 3H), 3.85 (s, 2H), 3.80 (s, 2H); ESI⁺ MS: m/z (rel intensity) 333.2 (100, [M+H]⁺).

Preparation of N-benzyl-4-((benzylamino)methyl)-N′-phenylbenzohydrazide (CS): A solution of N′-phenyl-4-((pyridin-2-ylmethylamino)methyl)-benzohydrazide, 4, (0.32 g, 0.96 mmol, 1.0 equiv.) in CH₂Cl₂ (10 mL) was treated with benzene sulfonyl chloride (135 μL, 1.05 mmol, 1.1 equiv.) and triethylamine (270 μL, 1.92 mmol, 2.0 equiv.). The resulting mixture was warmed stirred at room temperature for 4 h. A saturated aqueous solution of sodium bicarbonate (15 mL) was added. The product was extracted three times with 10 mL of CH₂Cl₂. The combined organic layers were dried over potassium carbonate, filtered and concentrated. The crude material was purified by chromatography using the ISCO (silica gel, 0-10% MeOH/CHCl₃) to afford 0.29 g (64% yield) of pure product: ¹H NMR (400 MHz, CDCl₃) [symbol] 8.62 (d, J=3.6 Hz, 1H), 8.27 (d, J=4.8 Hz, 1H), 7.77 (d, J=7.2 Hz, 2H), 7.62 (d, J=8.0 Hz, 1H), 7.54 (t, J=7.2 Hz, 1H), 7.47-7.42 (m, 3H), 7.19-7.14 (m, 4H), 7.04-7.00 (m, 1H), 6.88-6.80 (m, 3H), 6.38 (d, J=4.0 Hz, 1H), 4.44 (s, 2H), 4.41 (s, 2H); ESI⁺ MS: m/z (rel intensity) 473.1 (100, [M+H]⁺).

The following compounds CT, CU and CV were prepared using methods similar to those used to prepare compound CS (e.g., for CT, methanesulfonyl chloride was used in step g of Scheme XIII instead of phenylsulfonyl chloride; for CV, acetyl chloride was used instead of phenyl sulfonyl chloride in step g of Scheme XIII, and are also non-limiting examples of the second aspect of Embodiment I.

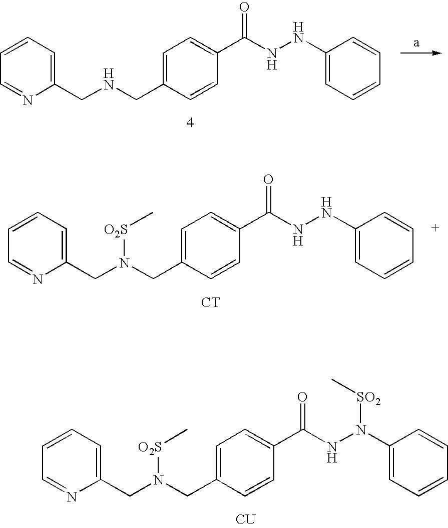

N′-phenyl-4-((N-(pyridin-2-ylmethyl)methan-11-ylsulfonamido)methyl)-benzohydrazide (CT): ¹H NMR (400 MHz, CDCl₃) [symbol] 8.54 (d, J=4.0 Hz, 1H), 8.46 (d, J=2.8 Hz, 1H), 7.77 (d, J=8.0 Hz, 2H), 7.61 (dt, J=1.6 Hz, J=7.6 Hz, 1H), 7.36 (d, J=8.0 Hz, 2H), 7.22-7.14 (m, 4H), 6.90-6.85 (m, 3H), 6.38 (d, J=3.6 Hz, 1H), 4.44 (s, 2H), 4.42 (s, 2H), 2.95 (s, 3H); ESI⁺ MS: m/z (rel intensity) 411.1 (100, [M+H]⁺).

N′-(methylsulfonyl)-N′-phenyl-4-((N-(pyridin-2-ylmethyl)methan-11-ylsulfonamido)methyl)benzohydrazide (CU): ¹H NMR (400 MHz, CDCl₃) [symbol] 9.25 (bs, 1H), 8.52 (m, 1H), 7.77 (d, J=8.4 Hz, 2H), 7.61 (dt, J=2.0 Hz, J=8.0 Hz, 1H), 7.56 (d, J=8.4 Hz, 2H), 7.38-7.32 (m, 4H), 7.28-7.14 (m, 3H), 4.44 (s, 2H), 4.41 (s, 2H), 3.31 (s, 3H), 2.97 (s, 3H); ESI⁺ MS: m/z (rel intensity) 489.1 (100, [M+H]⁺).

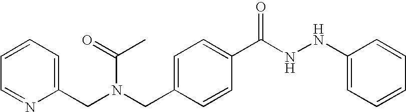

N′-phenyl-4-((N-(pyridin-2-ylmethyl)acetamido)methyl)benzohydrazide, (CV): ¹H NMR (400 MHz, CDCl₃) [symbol] 9.26 and 9.16 (s and s from rotomers, 1H), 8.53 and 8.45 (d and d from rotomers, J=4.0 Hz, 1H), 7.84 and 7.79 (d and d from rotomers, J=8.0 Hz, 2H), 7.65-7.56 (m, 1H), 7.27-7.05 (m, 4H), 6.87-6.81 (m, 3H), 6.47 (bs, 1H), 4.62 (s, 2H), 4.64 and 4.49 (s and s from rotomers, 2H), 2.16 and 2.11 (s and s from rotomers, 3H); ESI⁺ MS: m/z (rel intensity) 375.1 (100, [M+H]⁺).

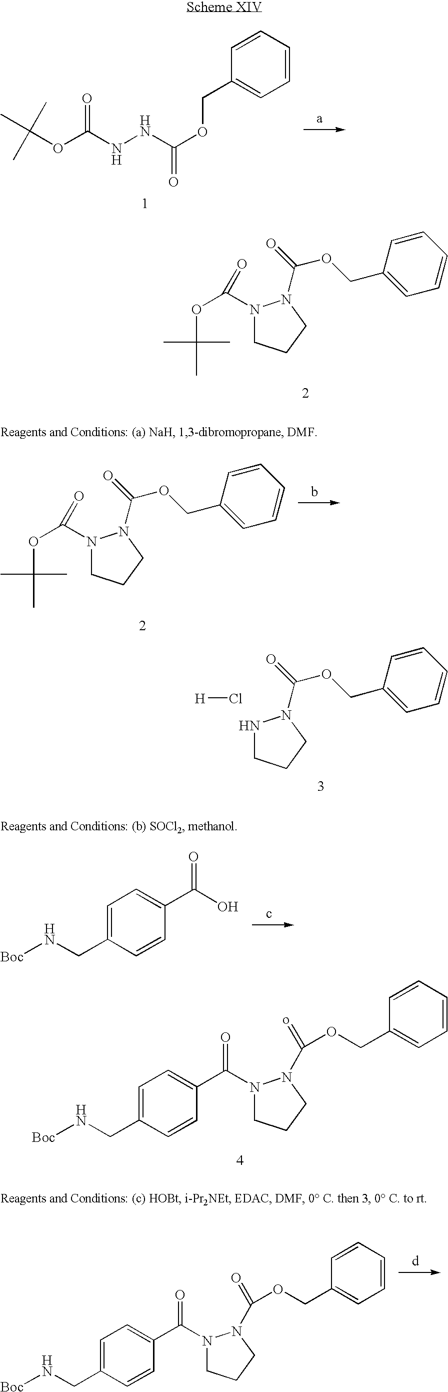

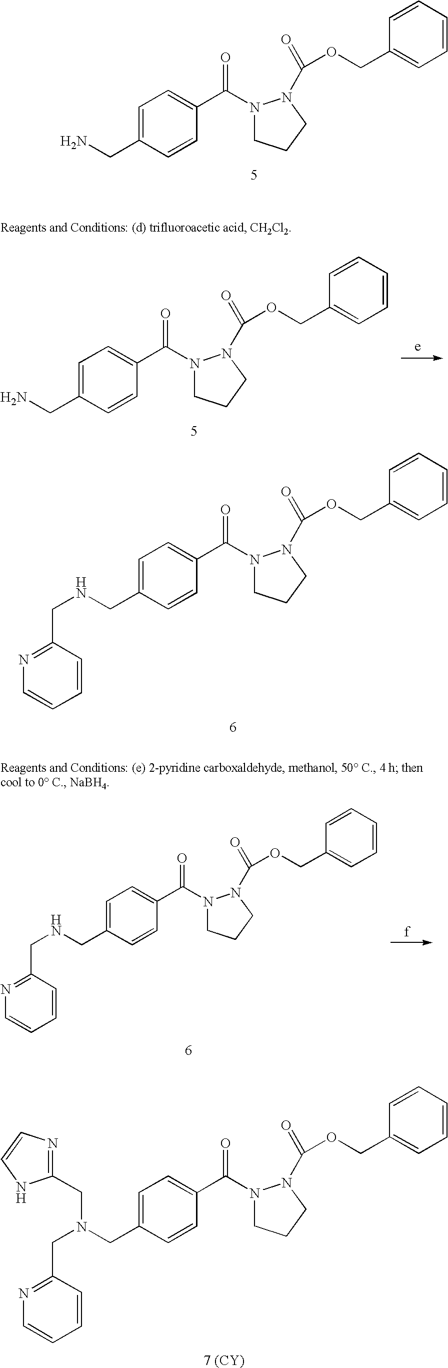

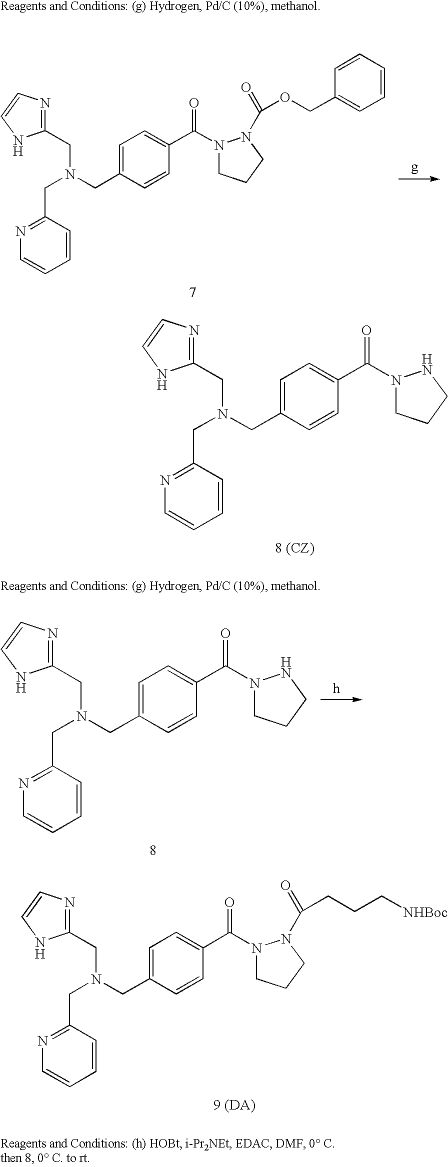

### Example 14

### (4-((((1H-imidazol-2-yl)methyl)(pyridin-2-ylmethyl)amino)methyl)phenyl)-(pyrazolidin-1-yl)methanone, 8, (CZ)

Preparation of pyrazolidine-1,2-dicarboxylic acid 1-benzyl ester 2-tert-butyl ester (2). To a solution of N-Cbz-N′-Boc-hydrazine (19.2 g, 72.2 mmol) in DMF (150 mL) is added portionwise NaH (5.8 g, 144.2 mmol, 60% dispersion in mineral oil). After stirring at room temperature for 1 h, a solution of 1,3-dibromopropane (7.3 mL, 72.2 mmol) in DMF (10 mL) is added dropwise. After stirring the reaction mixture at room temperature for 17 h, the mixture is diluted with aqueous saturated NH₄Cl solution and extracted twice with ether. The combined organic phases are washed twice with aqueous saturated NaCl solution, dried (MgSO₄), filtered and concentrated in vacuo to afford the desired product as a colorless oil which is used without further purification: ¹H NMR (400 MHz, d⁶-DMSO) [symbol] 7.33-7.27 (m, 5H), 5.15-5.01 (m, 2H), 3.79-3.68 (m, 2H), 3.18-3.03 (m, 2H), 1.98-1.87 (m, 2H), 1.31 (s, 9H).

Preparation of benzyl pyrazolidine-1-carboxylate hydrochloride (3). To a solution of crude pyrazolidine-1,2-dicarboxylic acid 1-benzyl ester 2-tert-butyl ester, 3, (23.0 g, 75.0 mmol) in methanol (260 mL) is added thionyl chloride (40 mL) dropwise. The reaction is warmed to room temperature and stirred an additional 16 hours. Concentration of the reaction mixture in vacuo affords a yellow oil. The oil is diluted with ether and stirred vigorously for 1 h, afterwhich the ether phase is decanted. The remaining residue is diluted with THF and the mixture stirred vigorously for 2 h to precipitate the desired product. The precipitate is filtered, washed with ether, dried and used without further purification: ¹H NMR (400 MHz, d⁶-DMSO) [symbol] 7.43-7.32 (m, 5H), 5.18 (s, 2H), 3.59 (t, J=6.8 Hz, 2H), 3.38 (t, J=6.8 Hz, 2H), 1.20-1.87 (tt, J=14.0, 6.8 Hz, 2H).

Preparation of benzyl 2-(4-((tert-butoxycarbonyl)methyl)benzoyl)-pyrazolidine-1-carboxylate (4). A solution of 4-((tert-butoxycarbonyl)methyl)benzoic acid (2.1 g, 8.3 mmol) in DMF (40 mL) at 0° C. was treated with HOBt (1.3 g, 9.9 mmol), Hünigs base (4.3 mL, 24.8 mmol) and then EDAC (1.9 g, 9.9 mmol). After 1 h at 0° C., benzyl pyrazolidine-1-carboxylate hydrochloride, 3, (2.0 g, 8.26 mmol) was added to the reaction and the mixture was slowly warmed to room temperature and stirred for an additional 18 h. The reaction mixture was diluted with aqueous saturated sodium bicarbonate and extracted with EtOAc. The organic phase was washed with twice with brine, dried over magnesium sulfate, filtered and concentrated in vacuo. The crude residue was purified by silica gel chromatography using the ISCO (0-5% MeOH/CH₂Cl₂) to afford 2.98 g of the desired product: ¹H NMR (400 MHz, d⁶-DMSO) [symbol] 7.47-7.42 (m, 3H), 7.32-7.26 (m, 3H), 7.22-7.18 (m, 4H), 5.04 (s, 2H), 4.13 (d, J=6.4 Hz, 2H), 3.88 (bd s, 2H), 3.36-3.21 (m, 2H), 2.05-2.00 (m, 2H), 1.37 (s, 9H); ESI⁺ MS: m/z (rel intensity) 440.1 (50, [M+H]⁺); 462.1 (100, [M+Na]⁺).

Preparation of benzyl 2-(4-(aminomethyl)benzoyl)pyrazolidine-1-carboxylate (5). To a solution of benzyl 2-(4-((tert-butoxycarbonyl)methyl)benzoyl)-pyrazolidine-1-carboxylate, 4, (2.9 g, 6.6 mmol) in CH₂Cl₂ (30 mL) at room temperature was added trifluoroacetic acid (20 mL) with stirring. After 1.5 h at room temperature, the reaction mixture was concentrated in vacuo. The crude residue was diluted with 1N NaOH solution and extracted with CHCl₃ and then 20% isopropanol/CHCl₃. The combined organic phases were dried over magnesium sulfate, filtered and concentrated in vacuo to afford 1.84 g of the desired product: ¹H NMR (400 MHz, d⁶-DMSO) [symbol] 7.46 (d, J=8.0 Hz, 2H), 7.32-7.26 (m, 5H), 7.20-7.18 (m, 2H), 5.05 (s, 2H), 3.79 (bd s, 2H), 3.38-3.25 (m, 4H), 2.03-2.00 (m, 2H); ESI⁺ MS: m/z (rel intensity) 440.1 (50, [M+H]⁺); 340.1 (100, [M+H]⁺).

Preparation of benzyl 2-(4-((pyridin-2-ylmethylamino)methyl)benzoyl)-pyrazolidine-1-carboxylate (6). To a solution of benzyl 2-(4-(aminomethyl)benzoyl)pyrazolidine-1-carboxylate, 5, (0.40 g, 1.18 mmol) in methanol (10 mL) was added 2-pyridine carboxaldehyde (0.12 mL, 1.24 mmol). The mixture was then heated to 50° C. After stirring 1.5 h at 50° C., the reaction mixture was cooled to 0° C. and NaBH₄ (0.07 g, 1.77 mmol) was added. After stirring 1 h at 0° C., the reaction mixture was quenched by pouring into aqueous saturated NaHCO₃. The aqueous phase was extracted with EtOAc. The organic phase was dried (MgSO₄), filtered and concentrated in vacuo. The crude residue was purified by silica gel chromatography (0-10% MeOH/CHCl₃) to afford 280 mg of the desired product: ¹H NMR (400 MHz, d⁶-DMSO) [symbol] 7.81 (d, J=7.6 Hz, 2H), 7.65 (t, J=7.6 Hz, 1H), 7.57-7.53 (m, 3H), 7.36-7.26 (m, 4H), 7.21 (bd s, 1H), 7.15-7.10 (m, 3H), 5.04 (s, 2H), 4.45 (s, 2H), 4.37 (s, 2H), 3.90-3.82 (m, 2H), 3.30-3.20 (m, 2H), 2.03-1.99 (m, 2H); ESI⁺ MS: m/z (rel intensity) 431.2 (100, [M+H]⁺).

Preparation of benzyl 2-(4-((((1H-imidazol-2-yl)methyl)(pyridin-2-ylmethyl)amino)methyl)benzoyl)pyrazolidine-1-carboxylate (7). To a solution of benzyl 2-(4-((pyridin-2-ylmethylamino)methyl)benzoyl)-pyrazolidine-1-carboxylate, 6, (0.20 g, 0.46 mmol) in 1,2-dichloroethane (6 mL) was added 2-imidazole carboxaldehyde (0.05 g, 0.53 mmol), sodium triacetoxyborohydride (0.15 g, 0.69 mmol) and glacial acetic acid (4 drops). The mixture was then heated to 60° C. After stirring 17 h at 60° C., the reaction mixture was quenched by pouring into aqueous saturated NaHCO₃. The aqueous phase was extracted with CHCl₃. The organic phase was dried (MgSO₄), filtered and concentrated in vacuo. The crude residue was purified by basic alumina gel chromatography (0-5% MeOH/CHCl₃) to afford 60 mg of the desired product: ESI⁺ MS: m/z (rel intensity) 511.2 (100, [M+H]⁺).

Preparation of (4-((((1H-imidazol-2-yl)methyl)(pyridin-2-ylmethyl)amino)-methyl)phenyl)-(pyrazolidin-1-yl)methanone, (8) (CZ). To a solution of benzyl 2-(4-((((1H-imidazol-2-yl)methyl)(pyridin-2-ylmethyl)amino)methyl)benzoyl)-pyrazolidine-1-carboxylate, 7, (0.50 g, 0.98 mmol) in methanol (10 mL) was added palladium (10 wt % on carbon, 80 mg). The reaction mixture was purged with hydrogen. The reaction was then stirred under hydrogen atmosphere for 17 h. The reaction mixture was filtered through celite and washed with methanol. The resulting filtrate was concentrated in vacuo to afford 370 mg of the desired product which was used without further purification: ¹H NMR (400 MHz, d⁶-DMSO) [symbol] 8.54-8.51 (m, 1H), 8.30-8.29 (m, 1H), 7.89-7.83 (m, 1H), 7.65-7.53 (m, 6H), 7.42-7.32 (m, 3H), 4.04 (s, 2H), 3.81 (s, 2H), 3.69 (s, 2H), 3.54-3.45 (bd s, 2H), 2.82-2.73 (m, 2H), 1.99-1.82 (bd s, 2H); ESI⁺ MS: m/z (rel intensity) 377.2 (100, [M+H]⁺).

Preparation of tert-butyl 4-(2-(4-((((1H-imidazol-2-yl)methyl)(pyridin-2-ylmethyl)amino)methyl)benzoyl)pyrazolidin-1-yl)-4-oxobutylcarbamate (9) (DA). To a solution of 4-tert-butoxycarbonylaminobutyric acid (0.12 g, 0.60 mmol) in DMF (4 mL) at 0° C. was added HOBt (0.97 g, 0.72 mmol), Hünigs base (0.32 mL, 1.80 mmol) and then EDAC (0.14 g, 0.72 mmol). After 1 h at 0° C., benzyl 2-(4-((((1H-imidazol-2-yl)methyl)(pyridin-2-ylmethyl)amino)methyl)benzoyl)-pyrazolidine-1-carboxylate, 7, (0.23 g, 0.60 mmol) was added to the reaction and the mixture was slowly warmed to room temperature and stirred for an additional 66 h. The reaction mixture was diluted with aqueous saturated sodium bicarbonate and extracted with EtOAc. The organic phase was washed with twice with brine, dried over magnesium sulfate, filtered and concentrated in vacuo. The crude residue was purified by basic alumina chromatography (0-10% MeOH/CHCl₃) to afford 100 mg of the desired product: ¹H NMR (400 MHz, d⁶-DMSO) [symbol] 8.46 (d, J=4.4 Hz, 1H), 7.75 (t, J=7.6 Hz, 1H), 7.62-7.57 (m, 3H), 7.51-7.49 (m, 2H), 7.22 (dd, J=6.8, 5.6 Hz, 2H), 7.04-7.02 (m, 1H), 6.84-6.75 (m, 2H), 3.95-3.89 (m, 1H), 3.65-3.44 (m, 7H), 3.12-3.05 (m, 1H)<2.85-1.99 (m, 2H), 2.12-1.88 (m, 3H), 1.58-1.49 (m, 2H), 1.34-1.29 (m, 2H), 1.27 (s, 9H); ESI⁺ MS: m/z (rel intensity) 562.3 (100, [M+H]⁺).

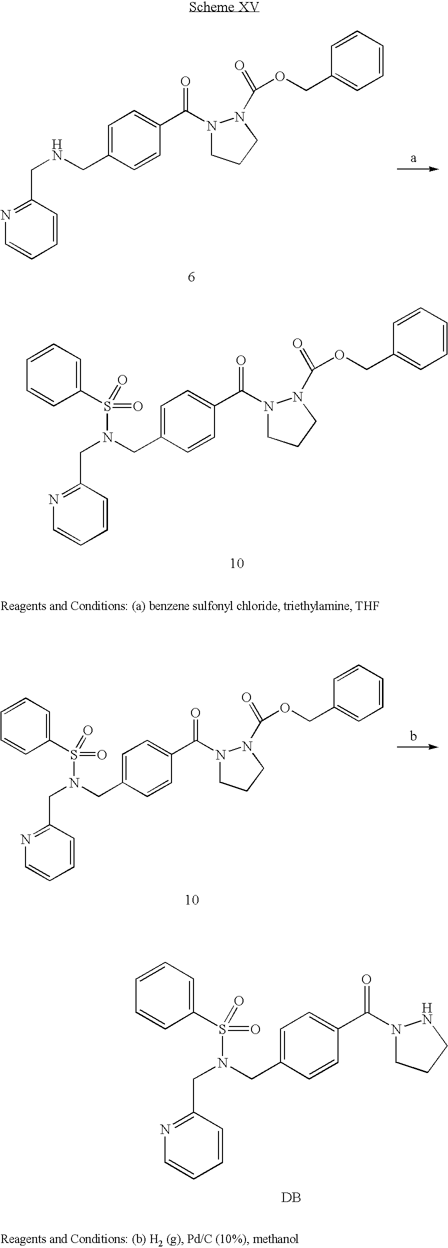

### Example 15

### N-(4-(pyrazolidine-1-carbonyl)benzyl)-N-(pyridin-2-ylmethyl)benzene-sulfonamide (DB)

Preparation of benzyl 2-(4-((N-(pyridin-2-ylmethyl)phenylsulfonamido)-methyl)benzoyl)pyrazolidine-1-carboxylate (10). To a solution of benzyl 2-(4-((pyridin-2-ylmethylamino)methyl)benzoyl)-pyrazolidine-1-carboxylate, 6, (0.21 g, 0.48 mmol) in THF (5 mL) was added benzene sulfonylchloride (0.07 mL, 0.57 mmol) followed by triethylamine (0.10 mL, 0.72 mmol). After stirring for 4 h at room temperature, the reaction mixture was diluted with aqueous saturated sodium bicarbonate and extracted with EtOAc. The organic phase was dried over magnesium sulfate, filtered and concentrated in vacuo. The crude residue was purified by silica gel chromatography (0-10% MeOH/CH₂Cl₂) to afford 225 mg of the desired product: ESI⁺ MS: m/z (rel intensity) 571.1 (100, [M+H]⁺).

Preparation of N-(4-(pyrazolidine-1-carbonyl)benzyl)-N-(pyridin-2-ylmethyl)benzenesulfonamide (DB). To a solution of benzyl 2-(4-((N-(pyridin-2-ylmethyl)phenylsulfonamido)-methyl)benzoyl)pyrazolidine-1-carboxylate, 10, (0.22 g, 0.39 mmol) in methanol (10 mL) was added palladium (10 wt % on carbon, 50 mg). The reaction mixture was purged with hydrogen. The reaction was then stirred under hydrogen atmosphere for 16 h. The reaction mixture was filtered through celite and washed with methanol. The resulting filtrate was concentrated in vacuo to afford 167 mg of the desired product which was used without further purification: ¹H NMR (400 MHz, d⁶-DMSO) [symbol] 8.31-8.29 (m, 1H), 7.80 (dd, J=8.4, 1.2 Hz, 2H), 7.64-7.48 (m, 6H), 7.12 (dd, J=7.6, 7.6 Hz, 4H), 5.34 (bd s, 1H), 4.44 (s, 2H), 4.37 (s, 2H), 3.46 (bd s, 2H), 2.75 (bd s, 2H), 1.92; ESI⁺ MS: m/z (rel intensity) 437.1 (100, [M+H]⁺).

Compounds in which L³ is —C(O)— (i.e., compounds BL-BO, BV, and BX) can be prepared as in Scheme IV, except that the appropriately substituted hydrazine is used in step c instead of phenyl hydrazine.

Compounds in which R¹ is other than H (i.e., compounds BP-CB, CO, and CQ) can be prepared by reaction, e.g., with the appropriate N-alkyl-N-aryl (or heteroaryl) disubstituted hydrazine using methods known in the art, e.g., analogous to the method shown in step e of Scheme VII.

Similarly, different groups Y can be prepared by the selection of the appropriately substituted hydrazine (e.g., in step b of Scheme X).

### Embodiment II

### Formula ID

### First Aspect

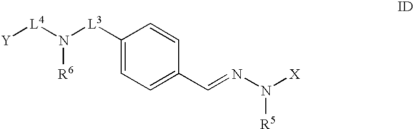

wherein Y-L⁴-N(R⁶)—, L³, X and R⁵ are defined herein below in Table 4.

TABLE 4

| Cmpd. | Y-L⁴-N(R⁶)- | L³ | X | R⁵ |
|---|---|---|---|---|
| DF | 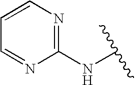 | CH₂ | 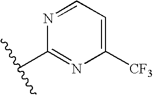 | H |
| DG | 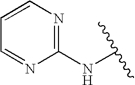 | CH₂ | 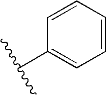 | CH₃ |
| DH | 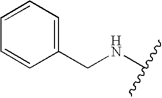 | CH₂ | 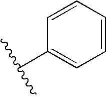 | CH₃ |
| DI | 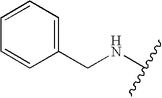 | CH₂ | 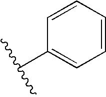 | H |
| DJ | 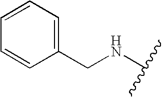 | CH₂ | 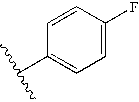 | H |
| DK | 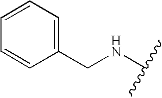 | CH₂ | 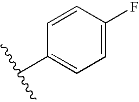 | H |
| DL | 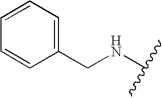 | CH₂ | 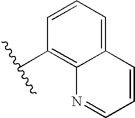 | H |
| DM | 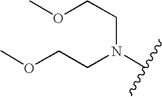 | CH₂ | 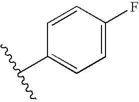 | H |
| DN | 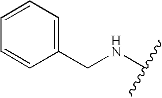 | CH₂ | 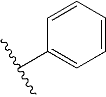 | H |
| DO | 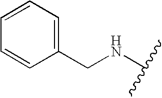 | CH₂ | 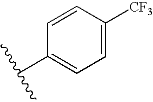 | H |
| DP | 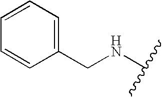 | CH₂ | 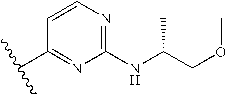 | H |
| DQ | 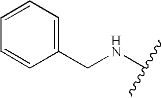 | CH₂ | 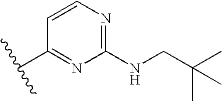 | H |
| DR | 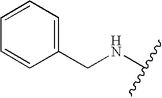 | CH₂ | 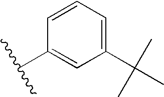 | H |
| DS | 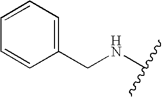 | CH₂ | 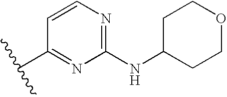 | H |
| DT | 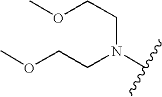 | CH₂ | 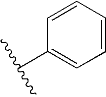 | H |
| DU | 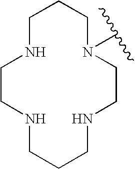 | CH₂ | 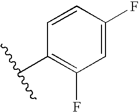 | H |
| DV | 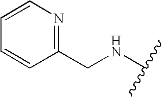 | CH₂ | 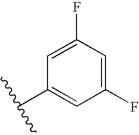 | H |
| DW | 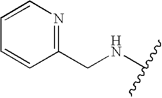 | CH₂ | 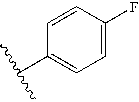 | H |
| DX | 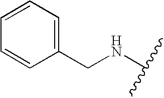 | CO | 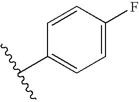 | H |
| DY | 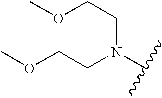 | CO | 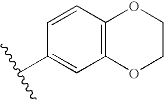 | H |
| DZ | 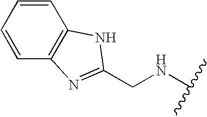 | CO | 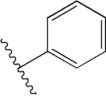 | H |
| EA | 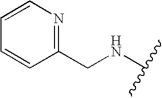 | CO | 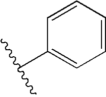 | H |
| EB | 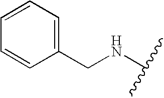 | CO | 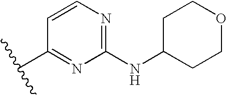 | H |

The compounds which comprise the first aspect of Embodiment II of the present invention can be prepared by the procedure outlined herein below in Scheme XVI.

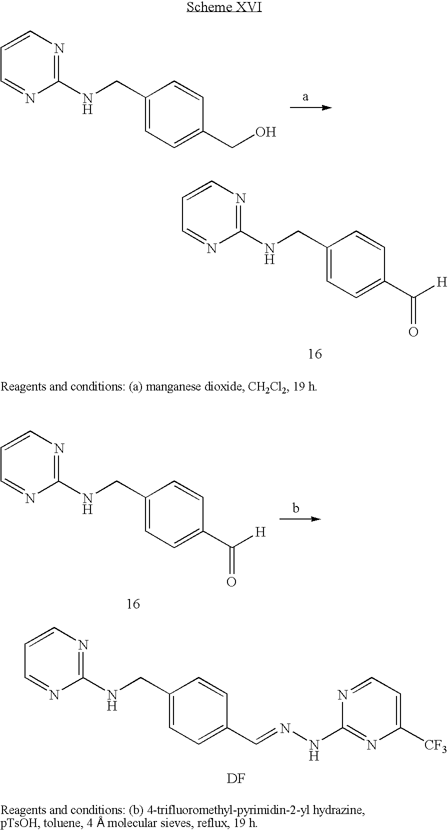

### Example 16

### 4-((benzylamino)methyl)-2-benzylidene-1-methylhydrazinyl)benzene (DF)

Preparation of 4-((pyrimidin-2-ylamino)methyl)benzaldehyde (16): To a solution of (4-((pyrimidin-2-ylamino)methyl)phenyl)methanol (prepared using the method of WO-2006-074428, herein incorporated by reference in its entirety) (4.4 g, 20.6 mmol) in a 3:1 mixture of CH₂Cl₂/DMF (100 mL) at room temperature was added manganese dioxide (10.7 g, 123.6 mmol). After stirring the reaction for 19 hours at room temperature, the mixture was filtered through celite and concentrated in vacuo to remove CH₂Cl₂. The residue was diluted with aqueous saturated NaHCO₃ and extracted with EtOAc. The organic layer was dried (MgSO₄), filtered and concentrated in vacuo. The crude residue was purified over silica (2% MeOH/CHCl₃ to 5% MeOH/CHCl₃) to afford 3.34 g of 16: ¹H NMR (400 MHz, d6-DMSO) δ 9.91 (s, 1H), 8.22 (d, J=4.4 Hz, 2H), 7.80 (d, J=8.4 Hz, 3H), 7.46 (d, J=8.0 Hz, 2H), 6.55 (t, J=4.8 Hz, 1H), 4.54 (d, J=6.0 Hz, 2H).

Preparation of hydrazone DF: To a solution of 4-((pyrimidin-2-ylamino)-methyl)benzaldehyde (0.50 g, 2.34 mmol) 16, 1-(4-(trifluoromethyl)pyrimidin-2-yl)hydrazine (0.42 g, 2.34 mmol), 4 Å molecular sieves (0.30 g) in toluene (12 mL) at room temperature was added para-toluene sulfonic acid (0.09 g, 0.47 mmol). The reaction was heated to reflux for 19 hours. The mixture was filtered through celite and concentrated in vacuo to remove CH₂Cl₂. The residue was dissolved in 25% methanol/EtOAc and washed with aqueous saturated NaHCO₃. The organic layer was dried (MgSO₄), filtered and concentrated in vacuo. The resulting residue was used without further purification: ¹H NMR (400 MHz, d₆-DMSO) δ 8.76 (d, J=4.8 Hz, 1H), 8.23 (d, J=4.8 Hz, 2H), 8.12 (s, 1H), 7.71 (t, J=6.0 Hz, 1H), 7.57 (d, J=8.0 Hz, 2H), 7.31 (d, J=8.4 Hz, 2H), 7.21 (d, J=4.8 Hz, 1H), 6.54 (t, J=4.8 Hz, 1H), 4.48 (d, J=6.4 Hz, 2H); ESI⁺ MS: m/z (rel intensity) 374 (100, M+H).

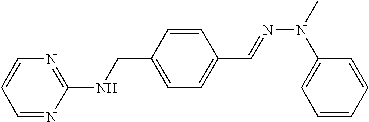

Preparation of (E)-N-(4-((2-methyl-2-phenylhydrazono)methyl)benzyl)pyrimidin-2-amine (DG): ¹H NMR (400 MHz, d₆-DMSO) δ 8.27 (d, J=4.8 Hz, 2H), 7.71 (t, J=5.1 Hz, 1H), 7.64 (d, J=8.8 Hz, 2H), 7.61 (s, 1H), 7.38 (d, J=7.6 Hz, 2H), 7.32-7.26 (m, 4H), 6.86 (t, J=7.6 Hz, 1H), 6.57 (t, J=4.8 Hz, 1H), 4.50 (d, J=6.0 Hz, 2H), 3.39 (s, 3H).

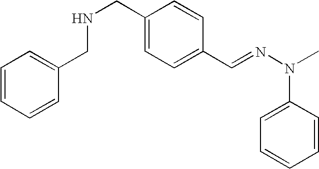

Preparation of (E)-N-benzyl-1-(4-((2-methyl-2-phenylhydrazono)methyl)phenyl)methanamine (DH): ¹H NMR (400 MHz, d₆-DMSO) δ 7.64 (d, J=8.4 Hz, 2H), 7.49 (s, 1H), 7.37 (dd, J=8.4, 1.2 Hz, 2H), 7.34-7.29 (m, 3H), 7.27-7.17 (m, 6H), 6.92 (t, J=7.2 Hz, 1H), 4.20 (m, 2H), 4.10 (m, 2H), 3.41 (s, 3H).

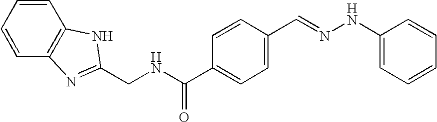

Preparation of (E)-N-((1H-benzo[d]imidazol-2-yl)methyl)-4-((2-phenylhydrazono)methyl)benzamide (DZ): ¹H NMR (400 MHz, d₆-DMSO) δ 12.29 (1H, bs), 10.57 (1H, s), 9.20 (1H, t, J=5.4 Hz), 7.97 (2H, d, J=8.4 Hz), 7.90 (1H, s), 7.75 (2H, d, J=8.8 Hz), 7.50 (2H, m), 7.24 (2H, t, J=7.6, 8 Hz), 7.13 (4H, m), 6.78 (1H, t, J=6.8, 7.6 Hz), 4.71 (2H, d, J=5.6 Hz).

Similarly, compounds in which Y-L⁴-N(R⁵)— is a bis(alkoxyalkyl)amine (e.g., compounds DM, DT, and DY), or in which Y is a group other than pyrimidine (e.g., compounds DI-DL, DN-DS, and DU-DX), can be prepared by replacing (4-((pyrimidin-2-ylamino)methyl)phenyl)methanol in step a with the appropriately substituted benzyl alcohol. Compounds in which L³ is —C(O)— (e.g., DN-DR) can be prepared by replacing (4-((pyrimidin-2-ylamino)methyl)phenyl)methanol in step a with 4-(hydroxymethyl)-N-(pyrimidin-2-yl)benzamide (or an analogous compound). Compounds in which R⁵ is a group other than H can be prepared, e.g., by alkylation of the corresponding hydrazone, or by use of the appropriately substituted hydrazine in step b.

### Embodiment III

### Formula IE

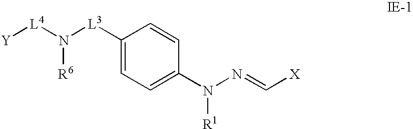

wherein Y-L⁴-N(R⁶)—, L³, R¹, and X are defined herein below in Table 5.

TABLE 5

| Cmpd. | Y-L⁴-N(R⁶)- | L³ | R¹ | X |
|---|---|---|---|---|
| EC | 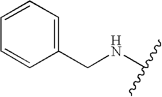 | CH₂ | CH₃ | 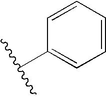 |
| ED | 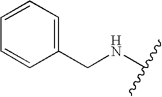 | CH₂ | CH₃ | 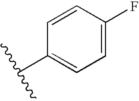 |
| EE | 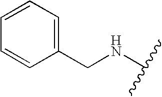 | CH₂ | 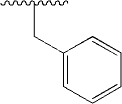 | 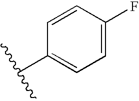 |
| EF | 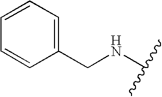 | CH₂ | CH₃ | 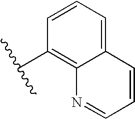 |
| EG | 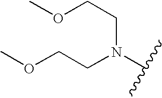 | CH₂ | CH₃ | 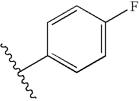 |
| EH | 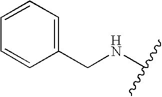 | CH₂ | H | 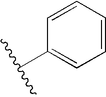 |
| EI | 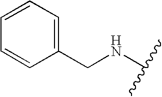 | CH₂ | H | 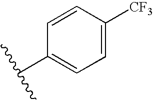 |
| EJ | 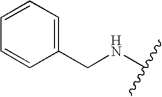 | CH₂ | CH₃ | 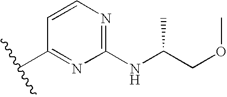 |
| EK | 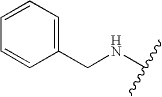 | CH₂ | CH₃ | 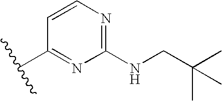 |
| EL | 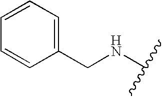 | CH₂ | H | 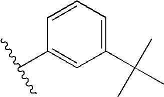 |
| EM | 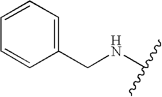 | CH₂ | H | 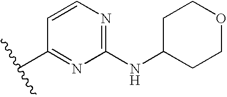 |
| EN | 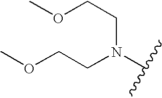 | CH₂ | H | 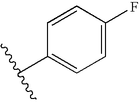 |
| EO | 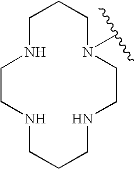 | CH₂ | H | 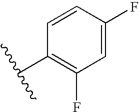 |
| EP | 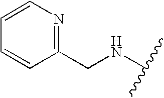 | CH₂ | H | 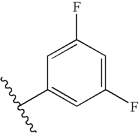 |
| EQ | 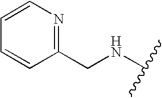 | CH₂ | H | 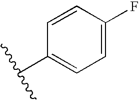 |
| ER | 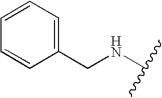 | CO | CH₃ | 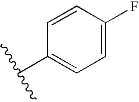 |
| ES | 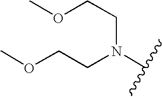 | CO | H | 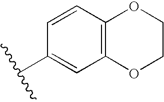 |
| ET | 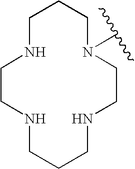 | CO | H | 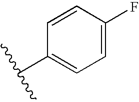 |
| EU | 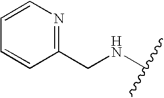 | CO | H | 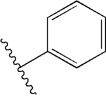 |
| EV | 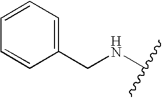 | CO | H | 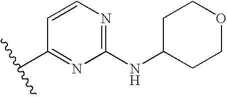 |

The compounds which comprise the first aspect of Embodiment II of the present invention can be prepared by the procedure outlined herein below in Scheme VII.

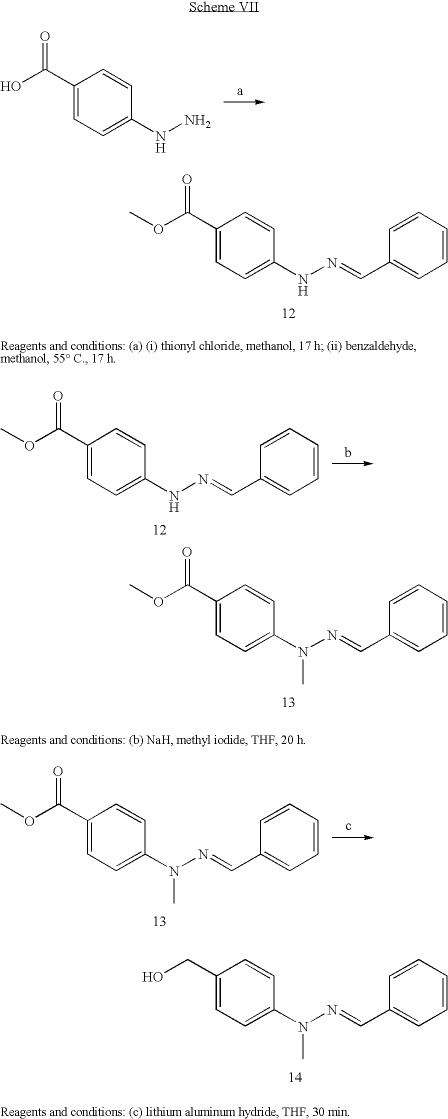

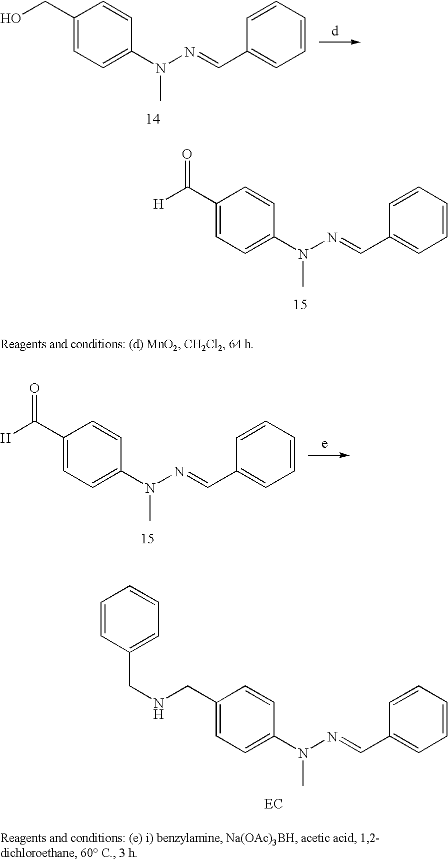

### Example 17

### 4-((benzylamino)methyl)-2-benzylidene-1-methylhydrazinyl)benzene (EC)

Preparation of methyl 4-(2-benzylidenehydrazinyl)benzoate (12): To a solution of 4-hydrazinobenzoic acid (5.0 g, 32.8 mmol) in methanol (40 mL) at room temperature was added dropwise thionyl chloride (4 mL). After stirring the reaction for 19 hours at room temperature, the mixture was concentrated in vacuo. The crude methyl ester (2.0 g, 9.9 mmol) was dissolved in methanol (50 mL) and benzaldehyde (1.0 mL, 9.9 mmol) was added to the mixture. The reaction was heated at 55° C. for 19 h. The solution was poured into aqueous saturated NaHCO₃ and extracted twice with EtOAc. The organic phase was dried (MgSO₄), filtered and concentrated in vacuo to yield 12, which was used without further purification: ¹H NMR (400 MHz, d₆-DMSO) δ 10.88 (s, 1H), 7.96 (s, 1H), 7.83 (d, J=8.8 Hz, 2H), 7.70 (dd, J=8.0, 1.6 Hz, 2H), 7.41 (t, J=8.0 Hz, 2H), 7.36-7.33 (m, 1H), 7.11 (d, J=8.0 Hz, 2H), 3.75 (s, 3H).

Preparation of methyl 4-(2-benzylidene-1-methylhydrazinyl)benzoate (13): To a solution of methyl 4-(2-benzylidenehydrazinyl)benzoate, 12, (0.80 g, 3.15 mmol) in THF (12 mL) was added sodium hydride (0.13 g, 3.15 mmol). The mixture was stirred at room temperature for 15 min before adding iodomethane (0.22 mL, 3.46 mmol) dropwise. After stirring the reaction for 20 hours at room temperature, the solution was poured into aqueous saturated NH₄Cl and extracted with EtOAc. The organic phase was dried (MgSO₄), filtered and concentrated in vacuo. The residue was purified over silica (10% EtOAc/hexanes to 100% EtOAc) to afford 700 mg of 13: ¹H NMR (400 MHz, d₆-DMSO) δ 7.85 (d, J=8.0 Hz, 2H), 7.72 (d, J=7.2 Hz, 2H), 7.47 (d, J=8.8 Hz, 2H), 7.39 (t, J=7.2 Hz, 2H), 7.30 (t, J=7.2 Hz, 1H), 3.77 (s, 3H), 3.45 (s, 3H).

Preparation of 2-benzylidene-1-(4-hydroxymethylphenyl)-1-methylhydrazine (14): To a solution of methyl 4-(2-benzylidene-1-methylhydrazinyl)benzoate, 13, (1.3 g, 4.9 mmol) in THF (40 mL) was added dropwise lithium aluminum hydride (9.7 mL of a 1M solution in THF, 9.7 mmol). After stirring the reaction at room temperature for 30 minutes, the mixture was cooled to 0° C. Water (0.4 mL) was added dropwise and the resulting solution was stirred at room temperature for 5 minutes. Aqueous NaOH (0.4 mL of 1N solution) was added to the mixture, which was then stirred at room temperature for 5 minutes. An additional 1.5 mL of water was added to the mixture. The reaction mixture was diluted with EtOAc, and the resulting organic phase was dried (MgSO₄), filtered and concentrated in vacuo to afford 1.04 g of 14, which was used without further purification: ¹H NMR (400 MHz, d₆-DMSO) δ 7.69 (d, J=7.2 Hz, 2H), 7.66 (s, 1H), 7.41-7.33 (m, 4H), 7.29-7.23 (m, 3H), 5.05 (t, J=5.6 Hz, OH), 4.42 (d, J=5.6 Hz, 2H), 3.40 (s, 3H).

Preparation of 4-(2-benzylidene-1-methylhydrazinyl)benzaldehyde (15): To a solution of 2-benzylidene-1-(4-hydroxymethylphenyl)-1-methylhydrazine, 14, (1.0 g, 4.3 mmol) in CH₂Cl₂ (40 mL) was added manganese dioxide (3.7 g, 43.0 mmol). After stirring the reaction at room temperature for 72 hours, the mixture was filtered through celite and washed with CH₂Cl₂. The filtrate was concentrated in vacuo to afford the desired product, which was used without further purification: ¹H NMR (400 MHz, d₆-DMSO) δ 9.84 (s, 1H), 7.82 (dd, J=8.8, 2.0 Hz, 2H), 7.72 (d, J=7.2 Hz, 2H), 7.65 (s, 1H), 7.47 (d, J=8.8 Hz, 2H), 7.39 (t, J=6.8 Hz, 2H), 7.36-7.31 (m, 1H), 3.49 (s, 3H).

Preparation of 4-((benzylamino)methyl)-2-benzylidene-1-methylhydrazinyl)benzene (EC): To a solution of 4-(2-benzylidene-1-methylhydrazinyl)benzaldehyde (0.18 g, 0.75 mmol), benzylamine (0.10 mL, 0.90 mmol) in 1,2-dichloroethane (5 mL) was added 1 drop of glacial acetic acid, followed by sodium triacetoxyborohydride (0.35 g, 1.65 mmol). The reaction mixture was heated to 60° C. and stirred for 3 hours. The mixture was diluted with aqueous saturated NaHCO₃ and extracted with EtOAc. The organic layer was dried (MgSO₄), filtered and concentrated in vacuo. The crude residue was purified over silica (10% EtOAc/hexanes to 100% EtOAc) to afford 203 mg of the desired product: ¹H NMR (400 MHz, d₆-DMSO) δ 8.35 (bd s, NH), 7.69 (d, J=7.2 Hz, 2H), 7.66 (s, 1H), 7.40-7.22 (m, 12H), 3.65 (s, 2H), 3.61 (s, 2H), 3.40 (s, 3H); ESI⁺ MS: m/z (rel intensity) 330 (32, M+H).

Similarly, compounds in which L³ is —C(O)— (i.e., compounds EO-ES) can be prepared by careful hydrolysis of ester 13 to the corresponding carboxylic acid, followed by activation of the acid and amide formation using the appropriate amine; X and Y can be varied by selection of the appropriate aldehyde (e.g., in step a) or amine (e.g., in step e); and R¹ can be varied by selection of the appropriate electrophile in step b.

### Preparation of Compounds of Formula (IF)

Compounds of Formula (IF), below:

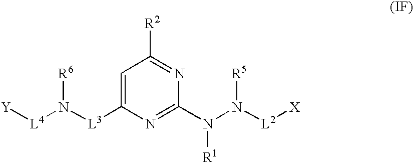

can be prepared by the general methods described below in General Scheme F.

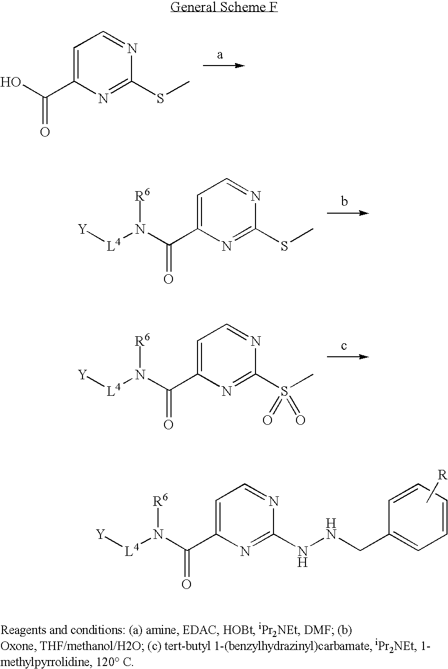

For example, compounds of formula (IF) can be prepared by reacting a substituted or unsubstituted 2-methylthiopyrimidine-4-carboxylic acid with an amine, to form a pyrimidine-4-carboxamide (step a). The pyrimidine-4-carboxamide can then be oxidized to the corresponding sulfonylpyrimidine-4-carboxamide (step b) and then reacted with an appropriately substituted hydrazinylcarbamate (step c).

It will be recognized that this general procedure can be modified in numerous ways to provide the compounds of the present invention, for example by varying the substitution of the starting 2-methylthiopyrimidine-4-carboxylic acid, selection of the amine in step a, and selection of the appropriately substituted hydrazinyl carbamate in step c.

### Embodiment IV

### Formula IG-1

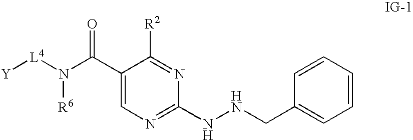

wherein R², R⁶, and -L⁴-Y are defined herein below in Table 6.

TABLE 6

| Cmpd. | R² | R⁶ | -L⁴-Y |
|---|---|---|---|
| EV | CF₃ | H | benzyl |
| EW | H | H | benzyl |
| EX | CH₂CH₃ | H | benzyl |
| EY | CH₃ | H | benzyl |
| EZ | CF₃ | H | 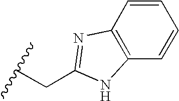 |
| FA | CF₃ | H | 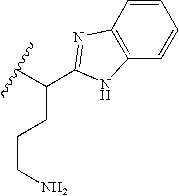 |
| FB | CF₃ | CH₃ | benzyl |
| FC | CF₃ | H | 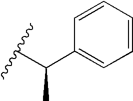 |
| FD | CF₃ | CH₃ | 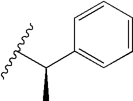 |
| FE | CF₃ | CH₃ | 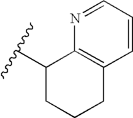 |
| FF | CF₃ | H | 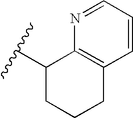 |
| FG | CF₃ | 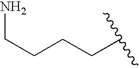 | 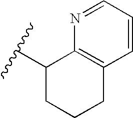 |
| FH | CF₃ | 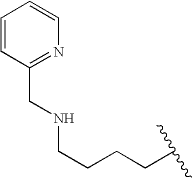 | 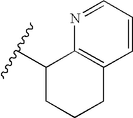 |

The compounds which comprise the first aspect of Embodiment IV of the present invention can be prepared by the procedure outlined herein below in Scheme XVIII.

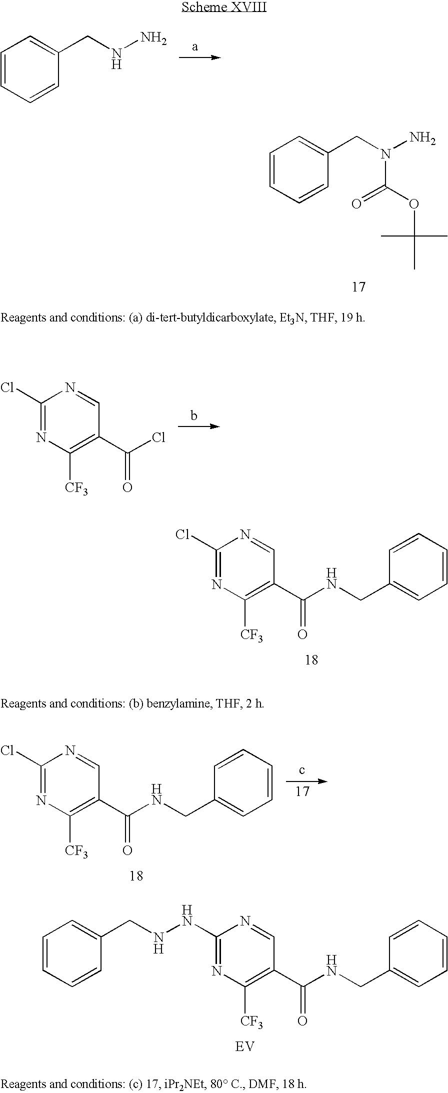

### Example 18

### N-benzyl-2-(2-benzylhydrazinyl)-4-(trifluoromethyl)pyrimidine-5-carboxamide (EV)

Preparation of tert-butyl 1-(benzylhydrazinyl)carbamate (17): A solution of benzylhydrazine (5.0 g, 25.6 mmol) and ⁱPr₂NEt (9.9 g, 76.8 mmol) in 50 mL of CH₂Cl₂ was cooled to 0° C. and treated with di-tert-butyl dicarbonate (6.2 g, 28.2 mmol). The solution was stirred at 0° C. for 2 hrs and quenched with aqueous saturated NaHCO₃. The organic layer was washed with water, brine, dried (Na₂SO₄) and concentrated in vacuo to yield 6.0 g of 17 as a pale yellow oil: ¹H NMR (400 MHz, d₆-DMSO) δ 7.29 (m, 5H), 4.54 (s, 2H), 4.34 (s, 2H), 1.39 (s, 9H).

Preparation of N-benzyl-2-chloro-4-(trifluoromethyl)pyrimidine-5-carboxamide (18): A solution of 2-chloro-4-(trifluoromethyl)pyrimidine-5-carbonyl chloride (2.0 g, 8.2 mmol) in 20 mL of anhydrous THF, was treated with benzylamine (0.9 g, 8.2 mmol). After stirring at ambient temperature for 2 hours, the reaction mixture was concentrated in vacuo. The residue was taken up in 100 mL of ethyl acetate, washed with water, brine, dried (Na₂SO₄) and concentrated in vacuo. The crude solid was purified by silica gel chromatography (50% EtOAc/hexanes) to yield 1.7 g of 18 as a white solid: ¹H NMR (400 MHz, d₆-DMSO) δ 9.30 (t, J=5.8 Hz, 1H), 9.25 (s, 1H), 7.36 (m, 4H), 7.29 (m, 1H), 4.50 (d, J=5.8 Hz, 2H).

Preparation of N-benzyl-2-(2-benzylhydrazinyl)-4-(trifluoromethyl)pyrimidine-5-carboxamide (EV): A solution of N-benzyl-2-chloro-4-(trifluoromethyl)pyrimidine-5-carboxamide (0.5 g, 1.7 mmol), 18, and ⁱPr₂NEt (0.9 mL, 5.1 mmol) in 10 mL of anhydrous DMF, was treated with tert-butyl 1-(benzylhydrazinyl)carbamate (0.8 g, 2.6 mmol), 17. The resulting solution was warmed to 80° C. and stirred for 18 hours. After cooling to ambient temperature, the reaction mixture was quenched with water and extracted with EtOAc. The combined organic extracts were washed with water, brine, dried (Na₂SO₄) and concentrated in vacuo. The crude product was purified by silica gel chromatography (20% EtOAc/hexanes gradient to 50% EtOAc/hexanes) to yield 70 mg of EV as an off-white solid, along with 300 mg of tert-butyl 1-benzyl-2-(5-(benzylcarbamoyl)-4-(trifluoromethyl)pyrimidin-2-yl)hydrazinecarboxylate. Analytical data for the desired product, DS: ¹H NMR (400 MHz, d₆-DMSO) δ 10.60 (br s, 1H), 9.20 (t, J=5.6 Hz, 1H), 8.84 (s, 1H), 7.48 (m, 2H), 7.34 (m, 7H), 7.27 (m, 2H), 4.45 (d, J=5.8 Hz, 2H), 4.28 (br s, 2H).

### Embodiment V

### Formula IH-1

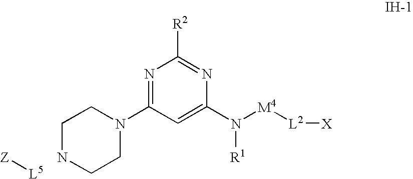

wherein R¹, R², R³ and R⁴ are defined herein below in Table 7.

TABLE 7

| No. | -L⁵-Z | R² | R¹ | -M⁴-L²-X |
|---|---|---|---|---|
| FJ | 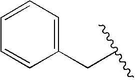 | H | H | 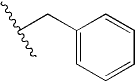 |
| FK | 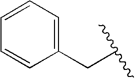 | H | H | 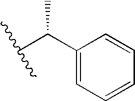 |
| FL | 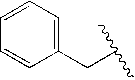 | H | H | 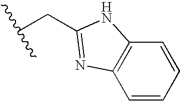 |
| FM | 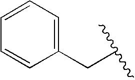 | H | H | 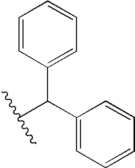 |
| FN | 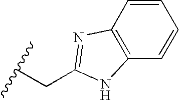 | CF₃ | H | 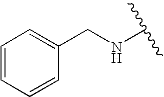 |
| FO | 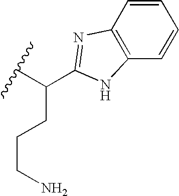 | CF₃ | H | 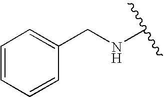 |

The compounds which comprise the first aspect of Embodiment V of the present invention can be prepared by the procedure outlined herein below in Scheme XIX.

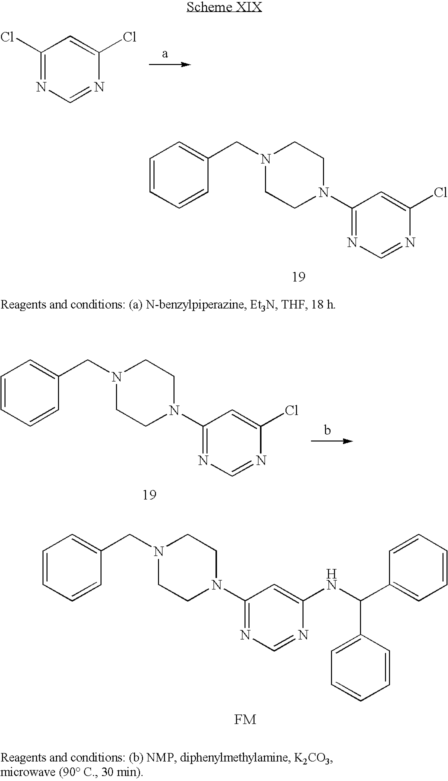

### Example 19

### N-benzhydryl-6-(4-benzylpiperazin-1-yl)pyrimidin-4-amine (FM)

Preparation of 4-(4-benzylpiperazin-1-yl)-6-chloropyrimidine (19): To a solution of 4,6-dichloropyrimidine (6.5 g, 43.7 mmol) in THF (60 mL) was added N-benzylpiperazine (8.0 mL, 45.9 mmol) followed by triethylamine (9.1 mL, 65.5 mmol). After stirring the mixture for 18 hours at room temperature, the solution was partitioned between EtOAc and saturated NaHCO₃ solution. The aqueous phase was extracted three times with EtOAc. The combined organic phases were washed with brine, dried (MgSO₄), filtered and concentrated in vacuo to afford 11.2 g of the crude product which was used without further purification: ¹H NMR (300 MHz, CDCl₃) δ 8.22 (s, 1H), 7.25-7.12 (m, 5H), 6.33 (s, 1H), 3.65-3.45 (m, 4H), 3.41 (s, 2H), 2.40-2.32 (br, 4H); ESI⁺ MS: m/z (rel intensity) 289 (100, M+H).

Preparation of N-benzhydryl-6-(4-benzylpiperazin-1-yl)pyrimidin-4-amine (FM): To a solution of 19 (0.29 g, 1.00 mmol) in NMP (3 mL) in a microwave reactor tube was added diphenylmethylamine (0.26 mL, 1.50 mmol) followed by potassium carbonate (0.41 g, 3.00 mmol). The tube was septum-sealed and subjected to microwave radiation in a CEM reactor (90° C., power 150 W, pressure max 250 psi) for 30 minutes. The solvents were decanted from the solid K₂CO₃ and then diluted to 10 mL with MeOH for purification by preparative HPLC (Polaris C18 column using acetonitrile/water with 0.1% TFA). The product fractions were free-based by partitioning with EtOAc/saturated NaHCO₃, washing with saturated NaHCO₃ solution, washing with brine and drying over MgSO₄. The residue was taken up in MeOH (3 mL) to which excess of a 2N HCl in Et₂O solution is added. Concentration in vacuo from CH₂Cl₂ as final solvent affords 81 mg (16%) of the desired product as the hydrochloride salt: ¹H NMR (300 MHz, d₆-DMSO) δ 8.30 (s, 1H), 7.60 (m, 2H), 7.49 (m, 2H), 7.38 (m, 8H), 7.32 (m, 2H), 6.13 (s, 1H), 4.35 (s, 2H), 3.45 (m, 8H), 3.09 (m, 1H), 2.76 (m, 1H); ESI⁺ MS: m/z (rel intensity) 436 (100, M+H).

The following are non-limiting examples of Embodiment V.

N-benzyl-6-(4-benzylpiperazin-1-yl)pyrimidin-4-amine (FJ): ¹H NMR (300 MHz, d₆-DMSO) δ 8.31 (s, 1H), 7.70 (br, NH), 7.55-7.45 (m, 6H), 7.4-7.25 (m, 4H), 5.98 (s, 1H), 4.54 (m, 4H), 4.37 (m, 4H), 2.40-2.32 (m, 4H); ESI⁺ MS: m/z (rel intensity) 360 (100, M+H).

(S)-6-(4-benzylpiperazin-1-yl)-N-(1-phenylethyl)pyrimidin-4-amine (FK): ¹H NMR (300 MHz, d₆-DMSO) δ 8.36 (s, 1H), 7.62 (m, 1H), 7.5-7.2 (m, 8H), 5.98 (s, 1H), 4.35 (m, 2H), 4.16 (m, 1H), 3.6-3.1 (m, 8H), 1.50 (d, J=5.0 Hz, 3H); ESI⁺ MS: m/z (rel intensity) 374 (100, M+H).

### N-((1H-benzo[d]imidazol-2-yl)methyl)-6-(4-benzylpiperazin-1-yl)pyrimidin-4-amine (EA)

### Embodiment VI

### Formula (II-1)

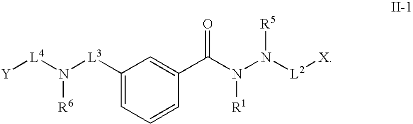

wherein Y-L⁴-N(R⁶)—, L², R¹, -L²-X, and R⁵ are defined herein below in Table 8.

TABLE 8

| Cmpd. | Y-L⁴-N(R⁶)- | L³ | R¹ | R⁵ | -L²-X |
|---|---|---|---|---|---|
| FP | 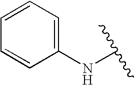 | CH₂ | H | H | 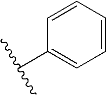 |
| FQ | 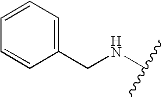 | CH₂ | H | H | 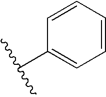 |
| FR | 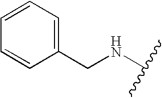 | CH₂ | CH₃ | H | H |
| FS | 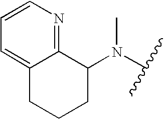 | CH₂ | CH₃ | H | H |
| FT | 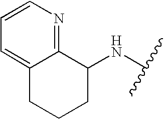 | CH₂ | CH₃ | H | H |
| FU | 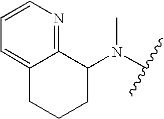 | CH₂ | H | H | 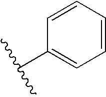 |

| | | | | |
|---|---|---|---|---|
| FV | 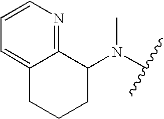 | CH₂ | 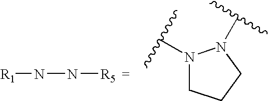 | H |
| FW | 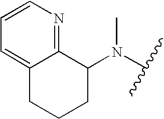 | CH₂ | 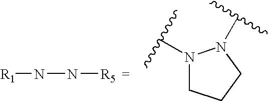 | 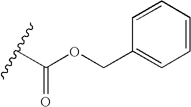 |
| FT | 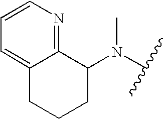 | CH₂ | 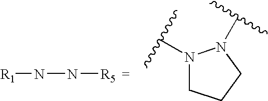 | 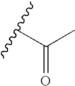 |
| FZ | 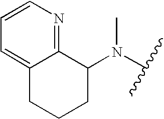 | CH₂ | 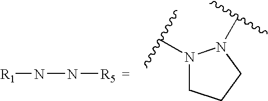 | 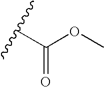 |
| GA | 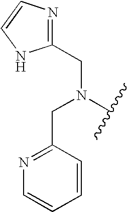 | CH₂ | 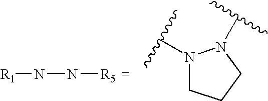 | H |
| GB | 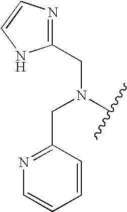 | CH₂ | 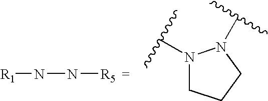 | 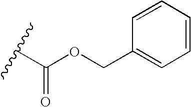 |
| GC | 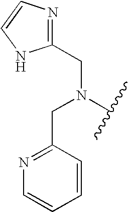 | CH₂ | 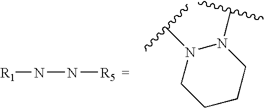 | H |

The compounds which comprise Embodiment VI of the present invention can be prepared by the procedures outlined in Schemes VII and XIV, corresponding to Embodiment I. However, instead of beginning with the para-substituted starting material in step a of Scheme VII, the meta-substituted starting material is used. Similarly, in step c of Scheme XIV, methyl 3-formylbenzoate is used instead of methyl 4-formylbenzoate.

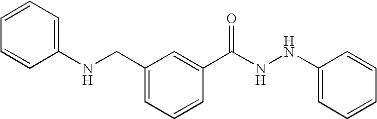

N′-phenyl-3-((phenylamino)methyl)benzohydrazide (FP): ¹H NMR (400 MHz, CDCl₃) δ 7.91 (s, 1H), 7.85 (s, 1H), 7.71 (d, J=7.20 Hz, 1H), 7.55 (d, J=7.20 Hz, H), 7.43 (t, J=7.20 Hz, 1H), 7.26-7.10 (m, 4H), 6.94-6.88 (m, 3H), 6.73 (t, J=7.60 Hz, 1H), 6.62 (d, J=7.60 Hz, 2H), 6.31 (bs, 1H), 4.39 (s, 4H); ESI⁺ MS: m/z (rel intensity) 318 (100, M+H).

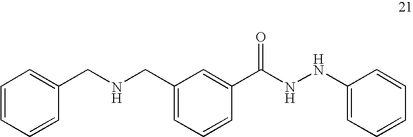

3-((benzylamino)methyl)-N′-phenylbenzohydrazide (FQ): ¹H NMR (400 MHz, CDCl₃) δ 8.02 (bs, 1H), 7.82 (s, 1H), 7.71 (d, J=7.20 Hz, 1H), 7.53 (d, J=7.20 Hz, H), 7.32 (d, J=5.60 Hz, 4H), 7.26-7.20 (m, 3H), 6.92-6.88 (m, 3H), 6.36 (br s, 1H), 3.85 (s, 2H), 63.81 (s, 2H); ESI⁺ MS: m/z (rel intensity) 685 (100, 2M+Na).

### Assays

Small molecule chemokine receptor modulation, agonism or antagonism, can be mediated by direct binding to the receptor affecting the signaling and chemotatic effects of the natural ligand for its receptor. In addition modulation can be obtained by interaction of the small molecule with effectors of the particular chemokine receptor pathway. For example, modulation of CXCR4 homodimerization (Rodriguez-Frade, et al., J. Cell. Biol. 1999; Mellado et al., Annual Review of Immunology 2001; Toth et al., J. Pharm. and Exp. Ther. 2004; Wang et al., Mol. Cancer. Ther. 2006), Heterodimerization with CCR2 (Percherancier, et al. JBC 2005, Sohy et al. JBC 2007) or CCR5 (Babcock, et al., JBC 2003) or CXCR7 (Sierro et al., PNAS 2007) or delta opioid receptor (DOR) (Pello et al European J of Imm. 2008, Hereld and Jin European J. of Imm. 2008), T cell receptor (Kumar et al., Immunity 2006). Modulation of the SDF-1/CXCR4 pathway can also be accomplished by modulation of GPR54/KISS receptor (Navenot et al., Cancer Res. 2005), cannabanoid receptor 2 (CB2R) (Coopman et al., International Immunopharmacology 2007), ZAP-70 tyrosine kinase (Ottoson et al., J. Immunology 2001) or sphingosine 1-phosphate receptors (Yopp et al., J. Immunology 2005).

### Assay 1: Test Compound Activity Against HIV Strains

A selected set of compounds are tested for their ability to inhibit the cellular entry of T-tropic HIV. The assay for this inhibition was carried out by Monogram Biosciences, Inc. using their well established Phenoscreen™ assay. Briefly, HIV strains of interest are tagged with a luciferase indicator gene to create an appropriate test vector. The test vector is amplified through transfection and the resulting virus is incubated in the presence of target host cells where intracellular florescence activity then becomes a measure of infection. Amplified virus is exposed to target host cells in the presence of a range of test drug concentrations to determine IC₅₀ measurements of entry inhibition. A modification of this test is further reapplied as a novel drug assay used in partnership with a number of pharmaceutical companies to test the effectiveness of novel entry inhibitors that target specific chemokines. It can used to detect activity against T-tropic, M-tropic, and dual-tropic viruses and Monogram Biosciences has a large bank of over 10,000 different virus strains to ultimately assess the range of applicability of our chemokine modulators. Certain compounds are tested to establish efficacy in a number of viral strains.

The compounds of the invention generally have an IC₅₀ value for viral entry inhibition in the one of the above HIV viral strains of interest of less than or equal to 100 micromolar. For example, compounds A, Z, AA, AB, AF, AL, AM, AO, and BE have IC₅₀ values of less than or equal to 10 micromolar.

### Assay 2: Screening for CXCR7 Activity

CXCR7 modulation activity was accessed using PathHunter™ β-Arrestin GPCR Assay Pharmacology from DiscoveRx using the protocol recommended by the manufacture for their CXCR7 β-Arrestin cell line. The compounds of the invention generally have an IC₅₀ value below 100 micromolar for CXCR7 modulation activity using this assay.

### Assay 3: Screening by Competition Assay Using Radiolabeled SDF-1

For radioligand binding competition test of CXCR4 or CXCR7, the following components are mixed in the wells of a 96 well plate (Master Block, Greiner, 786201) up to 100 μl assay buffer (50 mM HEPES; 5 mM MgCl₂; 1 mM CaCl2, 250 mM Sucrose, 100 mM NaCl and 0.5% BSA), compounds to be tested or 200-fold excess of cold ligand for non specific binding determination (SDF1-α R&D, 350-NS), radioligand [125I]-SDF-1α (PKI NEX346, 2200 Ci/mmol, diluted in assay buffer to give 0.03 nM) and 1 μg membrane extracts. The plate is incubated during 30 min at 37° C. in a water bath, filtered over GF/B filters (presoaked in 0.5% PEI for 1 h at room temperature) with a Filtermate Harvester (Perkin Elmer), and washed 6 times with 0.5 ml of ice cold filtration buffer (50 mM HEPES; 5 mM MgCl2; 1 mM CaCl2, 250 mM Sucrose, 0.5 M NaCl and 0.5% BSA). Following addition of 50 μl of Microscint 20 (Packard), and incubation during 15 min. on an orbital shaker, the plates are counted with a TopCount™ for 1 min/well. The compounds of the invention generally have an IC₅₀ value below 100 micromolar for competitive binding versus CXCR4 or CXCR7 activity using this assay.

Various references have been cited herein, each of which is incorporated herein by reference in its entirety for all purposes.

We claim:

1. A compound of formula (I), or a pharmaceutically acceptable salt, solvate, prodrug, enantiomer, diastereomer, tautomer, or ester thereof: 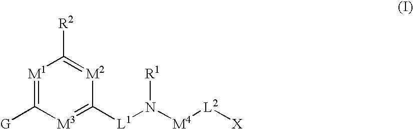
wherein
M¹ is ═N— or ═C(G)-;
M² and M³ are each independently ═N— or ═C(R³)—;
M⁴ is —C(R⁴)₂— or —N(R⁵)—;
each G is independently H, -L³-N(R⁶)-L⁴-Y, or a substituted or unsubstituted heterocyclyl of formula A: 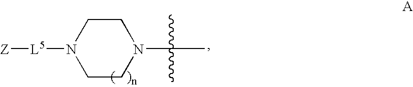
wherein n is an integer ranging from 1-3;
L¹ is selected from the group consisting of a covalent bond, —C(R⁴)₂—, —C(O)—, —S(O)₂—, —S(O)—, and —C(R⁴)═;
L² is selected from the group consisting of a covalent bond, —S(O)₂—, —S(O)—, —C(O)—, —C(O)O—, —C(R⁴)₂—, and —C(R⁴)═;
L³ is selected from the group consisting of —C(O)—, —C(R⁴)₂—, —S(O)₂—, and —S(O)—;
L⁴ is selected from the group consisting of a covalent bond, —C(R⁴)₂—, —C(O)—, —S(O)₂—, and —S(O)—;
L⁵ is selected from the group consisting of a covalent bond, —C(R⁴)₂—, —C(O)—, —S(O)₂—, and —S(O)—;
X is selected from the group consisting of H, alkyl, substituted or unsubstituted aryl, substituted or unsubstituted arylalkyl, substituted or unsubstituted heteroaryl, substituted or unsubstituted heterocyclyl, substituted or unsubstituted cycloalkyl, substituted or unsubstituted amino, substituted or unsubstituted aminoalkyl, and substituted or unsubstituted amido;
Y is selected from the group consisting of substituted or unsubstituted heteroaryl, substituted or unsubstituted aryl, hydroxyalkyl, alkoxyalkyl, substituted or unsubstituted aminoalkyl, substituted or unsubstituted cycloalkyl, substituted or unsubstituted —C(O)-heterocyclyl, substituted or unsubstituted amino, substituted or unsubstituted arylalkylamino, substituted or unsubstituted heteroarylalkylamino, amidoalkyl, substituted or unsubstituted amido, substituted or unsubstituted heterocyclyl, and alkyl;
Z is selected from the group consisting of H, alkyl, substituted or unsubstituted aryl, substituted or unsubstituted heteroaryl, alkoxyalkyl, and hydroxyalkyl;
R¹ is selected from the group consisting of H, alkyl, alkenyl, aminoalkyl, substituted or unsubstituted arylalkyl, substituted or unsubstituted —S(O)₂-aryl, substituted or unsubstituted —S(O)₂-heteroaryl, substituted or unsubstituted heteroarylalkyl, substituted or unsubstituted cycloalkyl, substituted or unsubstituted aryl, substituted or unsubstituted heteroaryl, acyl, carboxy, alkoxycarbonyl, and substituted or unsubstituted aminocarbonyl;
R² and each R³ are each independently selected from the group consisting of H, alkyl, halo, haloalkyl, alkoxy, hydroxy, acyl, substituted or unsubstituted amino, alkyl-S(O)ₘ—, substituted or unsubstituted amino-S(O)ₘ—, substituted or unsubstituted amido, carboxy, alkoxycarbonyl, substituted or unsubstituted aminocarbonyl, and —CN, wherein m is an integer ranging from 0-2;
each R⁴ is independently selected from the group consisting of H, alkyl, alkylthioalkyl, thioalkyl, substituted or unsubstituted aryl, substituted or unsubstituted aminoalkyl, and substituted or unsubstituted heteroaryl;
R⁵ is selected from the group consisting of H, substituted or unsubstituted heteroarylalkyl, alkyl, substituted or unsubstituted aminoalkyl, alkyl-S(O)₂—, substituted or unsubstituted cycloalkyl, substituted or unsubstituted arylalkyl, substituted or unsubstituted aryl, substituted or unsubstituted heteroaryl, substituted or unsubstituted heteroarylalkyl, acyl, carboxy, alkoxycarbonyl, and substituted or unsubstituted aminocarbonyl; or
R⁵ and R¹, together with the nitrogen atoms to which they are each attached, form a substituted or unsubstituted heterocyclyl ring; or
R⁵ and -L²-X, together with the nitrogen atom to which they are both attached, form a substituted or unsubstituted heterocyclyl ring;
R⁶ is selected from the group consisting of H, alkyl, substituted or unsubstituted aminoalkyl, substituted or unsubstituted arylalkyl, substituted or unsubstituted cycloalkyl, alkoxyalkyl, hydroxyalkyl, substituted or unsubstituted heteroarylalkyl, substituted or unsubstituted aryl, substituted or unsubstituted heteroaryl, acyl, carboxy, alkoxycarbonyl, and substituted or unsubstituted aminocarbonyl; or
R⁶ and -L⁴-Y, together with the nitrogen atom to which they are both attached, form a substituted or unsubstituted heterocyclyl ring;
with the following provisos:
(i) at least one G is not H;
(ii) when M⁴ is —N(R⁵)— and L² is —C(R⁴)═, then -M⁴-L²- is —N═C(R⁴)—;
(iii) when M⁴ is —C(R⁴)₂— and L² is —C(R⁴)═, then —N(R¹)-M⁴-L²- is —N(R¹)C(R⁴)═C(R⁴)—;
(iv) when L¹ is —C(R⁴)═, then -L¹-N(R¹)— is —C(R⁴)═N—;
(v) when M⁴ is —N(R⁵)— and G is a selected from the group consisting of substituted or unsubstituted piperazine, substituted or unsubstituted —CH₂-piperazine, and a substituted —CH₂-morpholine group, then X is not a 4-pyrimidinyl-2-cyano group; and
(vi) when M⁴ is —C(R⁴)₂—, then L¹ and L³ are both —C(O)— and X is not a substituted or unsubstituted imidazolyl or substituted or unsubstituted indolyl, or then at least one group G is a substituted or unsubstituted heterocyclyl of formula A.

2. The compound of claim 1 having the formula (IA):

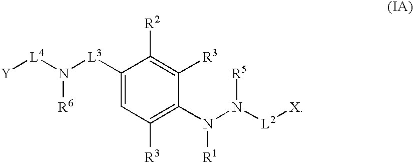

3. The compound of claim 2, wherein:
L² is selected from the group consisting of —S(O)₂—, —S(O)—, —C(O)—, and —C(R⁴)₂—;
L⁴ is —C(R⁴)₂—, —C(O)—, —S(O)—, or —S(O)₂—;
X is selected from the group consisting of substituted or unsubstituted aryl, substituted or unsubstituted heteroaryl, and substituted or unsubstituted cycloalkyl;
Y is selected from the group consisting of substituted or unsubstituted heteroaryl, substituted or unsubstituted aryl, hydroxyalkyl, alkoxyalkyl, amidoalkyl, amido, heterocyclyl, and aminoalkyl;
R¹ is selected from the group consisting of H, alkyl, substituted or unsubstituted arylalkyl, substituted or unsubstituted heteroarylalkyl, substituted or unsubstituted cycloalkyl, substituted or unsubstituted aryl, substituted or unsubstituted heteroaryl, acyl, carboxy, alkoxycarbonyl, aminoalkyl, and aminocarbonyl;
each R⁴ is independently selected from the group consisting of H, alkyl, alkylthioalkyl, thioalkyl, substituted or unsubstituted aryl, and substituted or unsubstituted heteroaryl;
R⁵ is selected from the group consisting of H, substituted or unsubstituted heteroarylalkyl, alkyl, substituted or unsubstituted cycloalkyl, substituted or unsubstituted arylalkyl, substituted or unsubstituted aryl, and substituted or unsubstituted heteroaryl; or
R⁵ and R¹, together with the nitrogen atoms to which they are each attached, form a substituted or unsubstituted heterocyclyl ring; and
R⁶ is selected from the group consisting of H, aminoalkyl, substituted or unsubstituted arylalkyl, substituted or unsubstituted cycloalkyl, alkoxyalkyl, hydroxyalkyl, substituted or unsubstituted heteroarylalkyl, substituted or unsubstituted aryl, and substituted or unsubstituted heteroaryl.

4. The compound of claim 1 having the formula (IB):

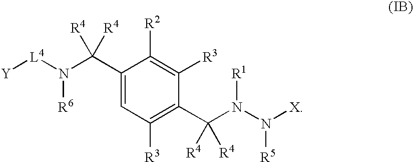

5. The compound of claim 4, wherein:
L⁴ is a covalent bond or —C(R⁴)₂—;
X is selected from the group consisting of substituted or unsubstituted aryl, and substituted or unsubstituted heteroaryl;
Y is selected from the group consisting of substituted or unsubstituted heteroaryl, substituted or unsubstituted aryl, alkoxyalkyl, and hydroxyalkyl;
R¹ is selected from the group consisting of H, alkyl, substituted or unsubstituted arylalkyl, substituted or unsubstituted —S(O)₂-aryl, substituted or unsubstituted heteroarylalkyl, substituted or unsubstituted cycloalkyl, substituted or unsubstituted aryl, and substituted or unsubstituted heteroaryl;
each R⁴ is independently selected from the group consisting of H, alkyl, substituted or unsubstituted aryl, and substituted or unsubstituted heteroaryl;
R⁵ is selected from the group consisting of H, substituted or unsubstituted heteroarylalkyl, alkyl, aminoalkyl, substituted or unsubstituted cycloalkyl, substituted or unsubstituted arylalkyl, substituted or unsubstituted aryl, and substituted or unsubstituted heteroaryl; and
R⁶ is selected from the group consisting of H, aminoalkyl, substituted or unsubstituted arylalkyl, substituted or unsubstituted cycloalkyl, alkoxyalkyl, hydroxyalkyl, substituted or unsubstituted heteroarylalkyl, substituted or unsubstituted aryl, substituted or unsubstituted heteroaryl, acyl, carboxy, alkoxycarbonyl, and aminocarbonyl; or
R⁶ and -L⁴-Y, together with the nitrogen atom to which they are both attached, form a substituted or unsubstituted heterocyclyl ring.

6. The compound of claim 1, having the formula (IC):

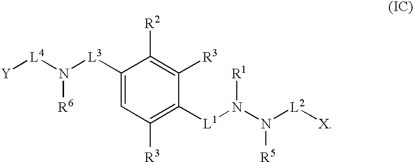

7. The compound of claim 6, wherein:
L¹ is selected from the group consisting of —C(R⁴)₂—, —C(O)—, —S(O)— and —S(O)₂—;
L² is selected from the group consisting of a covalent bond, —S(O)₂—, —S(O)—, —C(O)—, —C(O)O—, and —C(R⁴)₂—;
L⁴ is selected from the group consisting of a covalent bond, —C(R⁴)₂—, and —C(O)—;
X is selected from the group consisting of H, alkyl, substituted or unsubstituted aryl, substituted or unsubstituted heteroaryl, substituted or unsubstituted arylalkyl, amino, and aminoalkyl;
Y is selected from the group consisting of H, substituted or unsubstituted heteroaryl, substituted or unsubstituted aryl, hydroxyalkyl, alkoxyalkyl, substituted or unsubstituted —C(O)-heterocyclyl, substituted or unsubstituted arlyalkylamino, substituted or unsubstituted heteroarylalkylamino, and alkyl;
R¹ is selected from the group consisting of H, alkyl, substituted or unsubstituted arylalkyl, and substituted or unsubstituted heteroarylalkyl;
each R⁴ is independently selected from the group consisting of H, alkyl, alkylthioalkyl, and thioalkyl;
R⁵ is selected from the group consisting of H, substituted or unsubstituted heteroarylalkyl, alkyl, aminoalkyl, alkyl-S(O)₂—, substituted or unsubstituted cycloalkyl, and substituted or unsubstituted arylalkyl; or
R⁵ and -L²-X, together with the nitrogen atom to which they are both attached, form a substituted or unsubstituted heterocyclyl ring; or
R⁵ and R¹, together with the nitrogen atoms to which they are each attached, form a substituted or unsubstituted heterocyclyl ring; and
R⁶ is selected from the group consisting of H, substituted or unsubstituted arylalkyl, alkoxyalkyl, hydroxyalkyl, substituted or unsubstituted cycloalkyl, and substituted or unsubstituted heteroarylalkyl; or
R⁶ and -L⁴-Y, together with the nitrogen atom to which they are both attached, form a substituted or unsubstituted heterocyclyl ring.

8. The compound of claim 1, having the formula (IC-1): 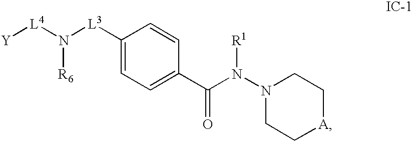
wherein A is —O— or —N(R⁵)—.

9. The compound of claim 8, wherein
L⁴ is selected from the group consisting of a covalent bond, —C(R⁴)₂—, —S(O)—, —S(O)₂—, and —C(O)—;
Y is selected from the group consisting of substituted or unsubstituted heteroaryl, substituted or unsubstituted aryl, hydroxyalkyl, and alkoxyalkyl;
R¹ is selected from the group consisting of H, alkyl, substituted or unsubstituted arylalkyl, aminoalkyl, and substituted or unsubstituted heteroarylalkyl;
each R⁴ is independently selected from the group consisting of H and alkyl; and
R⁶ is selected from the group consisting of H, alkoxyalkyl, hydroxyalkyl, substituted or unsubstituted arylalkyl, and substituted or unsubstituted heteroarylalkyl; or
R⁶ and -L⁴-Y, together with the nitrogen atom to which they are both attached, form a substituted or unsubstituted heterocyclyl ring.

10. The compound of claim 1, having the formula (IC-2):

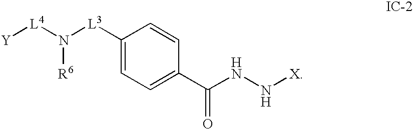

11. The compound of claim 10, wherein
L³ is selected from the group consisting of —C(R⁴)₂—, —S(O)—, —S(O)₂—, and —C(O)—;
L⁴ is selected from the group consisting of a covalent bond, —C(R⁴)₂—, and —C(O)—;
X is selected from the group consisting of substituted or unsubstituted aryl, and substituted or unsubstituted heteroaryl;
Y is selected from the group consisting of substituted or unsubstituted heteroaryl, substituted or unsubstituted aryl, hydroxyalkyl, alkoxyalkyl, and substituted or unsubstituted arylalkylamino;
each R⁴ is independently selected from the group consisting of H, alkyl, alkylthioalkyl, and thioalkyl;
R⁶ is selected from the group consisting of H, substituted or unsubstituted arylalkyl, alkoxyalkyl, hydroxyalkyl, and substituted or unsubstituted heteroarylalkyl; or
R⁶ and -L⁴-Y, together with the nitrogen atom to which they are both attached, form a substituted or unsubstituted heterocyclyl ring.

12. The compound of claim 1, having the formula (IC-3):

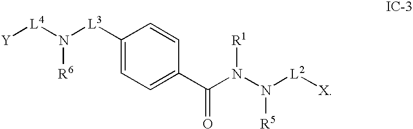

13. The compound of claim 12, wherein:
L² is selected from the group consisting of a covalent bond, —S(O)₂—, —S(O)—, —C(O)—, —C(O)O—, and —C(R⁴)₂—;
L₃ is —C(R⁴)₂— or —C(O)—;
L⁴ is selected from the group consisting of a covalent bond, —C(R⁴)₂—, —S(O)—, —S(O)₂—, and —C(O)—;
X is selected from the group consisting of H, alkyl, substituted or unsubstituted aryl, substituted or unsubstituted arylalkyl, substituted or unsubstituted heteroaryl, amino, and aminoalkyl;
Y is selected from the group consisting of H, substituted or unsubstituted heteroaryl, substituted or unsubstituted aryl, hydroxyalkyl, alkoxyalkyl, substituted or unsubstituted —C(O)-heterocyclyl, substituted or unsubstituted arlyalkylamino, substituted or unsubstituted heteroarylalkylamino, and alkyl;
R¹ is selected from the group consisting of H, alkyl, substituted or unsubstituted arylalkyl, and substituted or unsubstituted heteroarylalkyl;
each R⁴ is independently selected from the group consisting of H, alkyl, alkylthioalkyl, and thioalkyl;
R⁵ is selected from the group consisting of H, substituted or unsubstituted heteroarylalkyl, alkyl, alkyl-S(O)₂—, aminoalkyl, substituted or unsubstituted cycloalkyl, and substituted or unsubstituted arylalkyl; or
R₅ and R₁, together with the nitrogen atoms to which they are each attached, form a substituted or unsubstituted heterocyclyl ring; or
R⁵ and -L²-X, together with the nitrogen atom to which they are both attached, form a substituted or unsubstituted heterocyclyl ring; and
R⁶ is selected from the group consisting of H, substituted or unsubstituted cycloalkyl, substituted or unsubstituted arylalkyl, alkoxyalkyl, hydroxyalkyl, and substituted or unsubstituted heteroarylalkyl; or
R⁶ and -L⁴-Y, together with the nitrogen atom to which they are both attached, form a substituted or unsubstituted heterocyclyl ring.

14. The compound of claim 1, having the formula (ID):

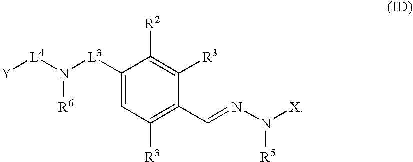

15. The compound of claim 14, wherein:
L⁴ is selected from the group consisting of a covalent bond, —C(R⁴)₂—, —S(O)—, —S(O)₂—, —C(O)—;
X and Y are independently selected from the group consisting of substituted or unsubstituted aryl, and substituted or unsubstituted heteroaryl;
each R⁴ is independently H or alkyl;
R⁵ is selected from the group consisting of H, substituted or unsubstituted heteroarylalkyl, alkyl, substituted or unsubstituted cycloalkyl, substituted or unsubstituted arylalkyl, substituted or unsubstituted aryl, and substituted or unsubstituted heteroaryl; and
R⁶ is selected from the group consisting of H, substituted or unsubstituted arylalkyl, substituted or unsubstituted cycloalkyl, substituted or unsubstituted heteroarylalkyl, substituted or unsubstituted aryl, and substituted or unsubstituted heteroaryl.

16. The compound of claim 1 having the formula (IE):

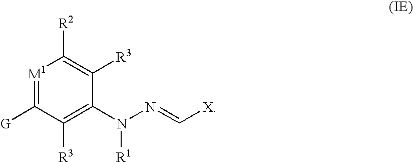

17. The compound of claim 16, wherein:
L⁴ is a covalent bond or —C(R⁴)₂—;
X is selected from the group consisting of substituted or unsubstituted aryl, and substituted or unsubstituted heteroaryl;
Y is selected from the group consisting of substituted or unsubstituted heteroaryl, substituted or unsubstituted aryl, and aminoalkyl;
R¹ is selected from the group consisting of H, alkyl, substituted or unsubstituted arylalkyl, substituted or unsubstituted heteroarylalkyl, substituted or unsubstituted aryl, and substituted or unsubstituted heteroaryl;
each R⁴ is independently H or alkyl; and
R⁶ is selected from the group consisting of H, substituted or unsubstituted arylalkyl, substituted or unsubstituted cycloalkyl, substituted or unsubstituted heteroarylalkyl, substituted or unsubstituted aryl, and substituted or unsubstituted heteroaryl.

18. The compound of claim 1, having the formula (IF):

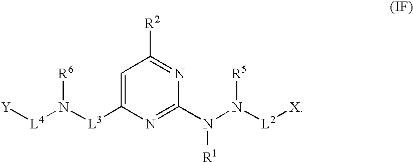

19. The compound of claim 18, wherein:
L² is selected from the group consisting of a covalent bond, —C(O)—, —S(O)—, —S(O)₂—, and —C(R⁴)₂—;
X is selected from the group consisting of substituted or unsubstituted aryl, and substituted or unsubstituted heteroaryl;
Y is selected from the group consisting of substituted or unsubstituted aryl, substituted or unsubstituted heteroaryl and amino;
R¹ is selected from the group consisting of H, alkyl, substituted or unsubstituted arylalkyl, and substituted or unsubstituted heteroarylalkyl;
each R⁴ is H or alkyl;
R⁵ is selected from the group consisting of H, substituted or unsubstituted heteroarylalkyl, alkyl, substituted or unsubstituted cycloalkyl, and substituted or unsubstituted arylalkyl; or
R⁵ and R¹, together with the nitrogen atoms to which they are each attached, form a substituted or unsubstituted heterocyclyl ring; and
R⁶ is selected from the group consisting of H, substituted or unsubstituted arylalkyl, substituted or unsubstituted cycloalkyl, and substituted or unsubstituted heteroarylalkyl.

20. The compound of claim 1, having the formula (IG):

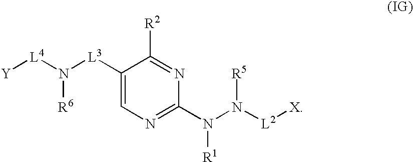

21. The compound of claim 20, wherein:
L² is —C(R⁴)₂— or a covalent bond;
L⁴ is selected from the group consisting of a covalent bond, —S(O)₂—, —S(O)—, and —C(R⁴)₂—;
X is selected from the group consisting of substituted or unsubstituted aryl, and substituted or unsubstituted heteroaryl;
Y is selected from the group consisting of substituted or unsubstituted aryl, substituted or unsubstituted cycloalkyl, substituted or unsubstituted arylalkyl, and substituted or unsubstituted heteroaryl;
R¹ is selected from the group consisting of H, alkyl, substituted or unsubstituted arylalkyl, and substituted or unsubstituted heteroarylalkyl;
each R⁴ is independently selected from the group consisting of H, alkyl, and aminoalkyl;
R⁵ is selected from the group consisting of H, substituted or unsubstituted heteroarylalkyl, alkyl, substituted or unsubstituted cycloalkyl, and substituted or unsubstituted arylalkyl; and
R⁶ is selected from the group consisting of H, alkyl, aminoalkyl, substituted or unsubstituted arylalkyl, substituted or unsubstituted cycloalkyl, and substituted or unsubstituted heteroarylalkyl.

22. The compound of claim 1, having the formula (IG-1):

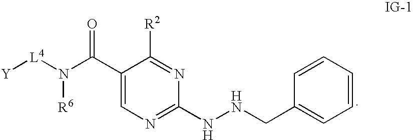

23. The compound of claim 22, wherein:
L⁴ is a covalent bond or —C(R⁴)₂—;
Y is selected from the group consisting of substituted or unsubstituted aryl, substituted or unsubstituted cycloalkyl, substituted or unsubstituted arylalkyl, and substituted or unsubstituted heteroaryl;
R² is selected from the group consisting of H, alkyl, and haloalkyl;
each R⁴ is independently selected from the group consisting of H, alkyl, and aminoalkyl; and
R⁶ is selected from the group consisting of H, alkyl, and aminoalkyl.

24. The compound of claim 1, having the formula (IH):

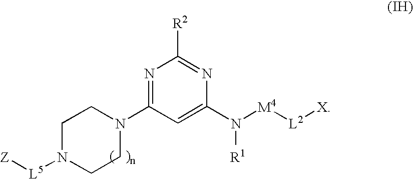

25. The compound of claim 24, wherein:
M⁴ is —N(R⁵)— or C(R⁴)₂—;
L² is selected from the group consisting of a covalent bond, —S(O)₂—, —S(O)—, —C(O)—, and —C(R⁴)₂—;
L⁵ is —C(R⁴)₂, —C(O)—, —S(O)₂—, or —S(O)—;
X is selected from the group consisting of substituted or unsubstituted aryl, and substituted or unsubstituted heteroaryl;
Z is selected from the group consisting of substituted or unsubstituted aryl, and substituted or unsubstituted heteroaryl;
R¹ is selected from the group consisting of H, alkyl, substituted or unsubstituted arylalkyl, substituted or unsubstituted heteroarylalkyl, and substituted or unsubstituted cycloalkyl;
each R⁴ is independently selected from the group consisting of H, alkyl, and substituted or unsubstituted aryl;
R⁵ is selected from the group consisting of H, substituted or unsubstituted heteroarylalkyl, alkyl, substituted or unsubstituted cycloalkyl, substituted or unsubstituted arylalkyl; and
n is 1 or 2.

26. The compound of claim 1, having the formula (IH-1):

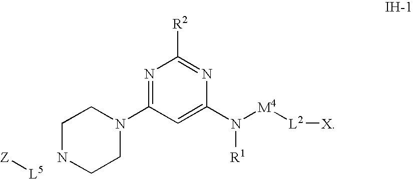

27. The compound of claim 26, wherein:
M⁴ is —C(R⁴)₂—;
L² is selected from the group consisting of a covalent bond, —S(O)₂—, —C(O)—, and —C(R⁴)₂—;
L⁵ is —C(R⁴)₂, —C(O)—, —S(O)₂—, or —S(O)—;
X is selected from the group consisting of substituted or unsubstituted aryl, and substituted or unsubstituted heteroaryl;
Z is selected from the group consisting of substituted or unsubstituted aryl, and substituted or unsubstituted heteroaryl;
R¹ is selected from the group consisting of H, alkyl, substituted or unsubstituted arylalkyl, and substituted or unsubstituted heteroarylalkyl;
R² is selected from the group consisting of H, alkyl, and haloalkyl; and
each R⁴ is independently selected from the group consisting H, alkyl, aminoalkyl, and substituted or unsubstituted aryl.

28. The compound of claim 1 having the formula (II):

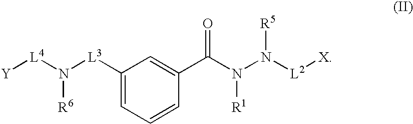

29. The compound of claim 28, wherein:
L² is selected from the group consisting of a covalent bond, —S(O)₂—, —S(O)—, —C(O)—, —C(O)O—, —C(R⁴)₂—, and —C(R⁴)═;
L³ is selected from the group consisting of —C(O)—, —C(R⁴)₂—, —S(O)₂—, and —S(O)—;
L⁴ is selected from the group consisting of a covalent bond, —C(R⁴)₂—, —C(O)—, —S(O)₂—, and —S(O)—;
X is selected from the group consisting of H, alkyl, substituted or unsubstituted aryl, substituted or unsubstituted arylalkyl, substituted or unsubstituted heteroaryl, substituted or unsubstituted heterocyclyl, substituted or unsubstituted cycloalkyl, amino, and amido;
Y is selected from the group consisting of substituted or unsubstituted heteroaryl, substituted or unsubstituted aryl, hydroxyalkyl, alkoxyalkyl, aminoalkyl, substituted or unsubstituted cycloalkyl, substituted or unsubstituted arylalkyl, substituted or unsubstituted —C(O)-heterocyclyl, amino, substituted or unsubstituted arylalkylamino, substituted or unsubstituted heteroarylalkylamino, amidoalkyl, amido, substituted or unsubstituted heterocyclyl, and alkyl;
R¹ is selected from the group consisting of H, alkyl, alkenyl, aminoalkyl, substituted or unsubstituted arylalkyl, substituted or unsubstituted —S(O)₂-aryl, substituted or unsubstituted —S(O)₂-heteroaryl, substituted or unsubstituted heteroarylalkyl, substituted or unsubstituted cycloalkyl, substituted or unsubstituted aryl, substituted or unsubstituted heteroaryl, acyl, carboxy, alkoxycarbonyl, and aminocarbonyl;
R⁵ is selected from the group consisting of H, substituted or unsubstituted heteroarylalkyl, alkyl, aminoalkyl, substituted or unsubstituted cycloalkyl, substituted or unsubstituted arylalkyl, substituted or unsubstituted aryl, substituted or unsubstituted heteroaryl, acyl, carboxy, alkoxycarbonyl, and aminocarbonyl; or
R⁵ and R¹, together with the nitrogen atoms to which they are each attached, form a substituted or unsubstituted heterocyclyl ring; or
R⁶ is selected from the group consisting of H, alkyl, aminoalkyl, substituted or unsubstituted arylalkyl, substituted or unsubstituted cycloalkyl, alkoxyalkyl, hydroxyalkyl, substituted or unsubstituted heteroarylalkyl, substituted or unsubstituted aryl, substituted or unsubstituted heteroaryl, acyl, carboxy, alkoxycarbonyl, and aminocarbonyl.

30. A compound of claim 1, selected from the group consisting of: 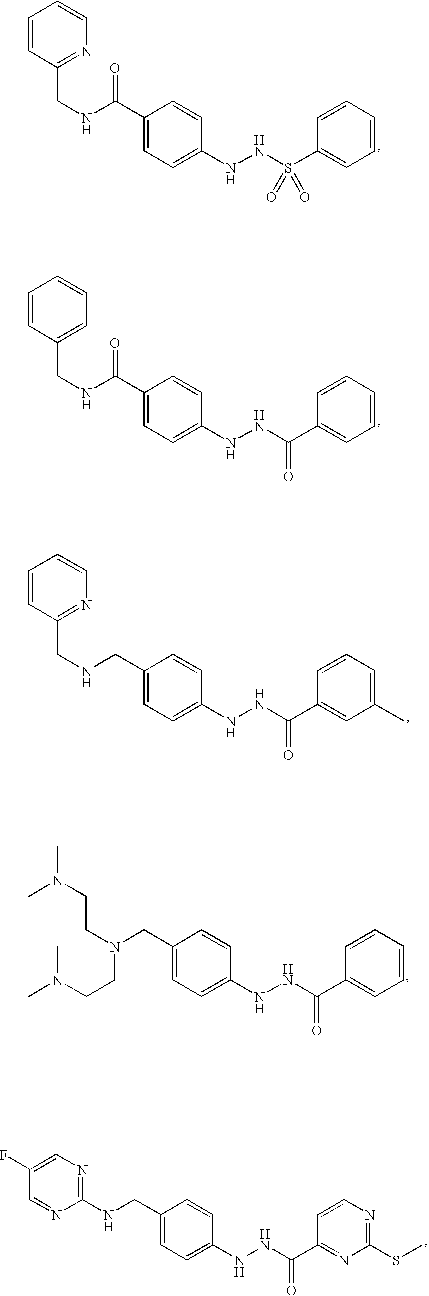 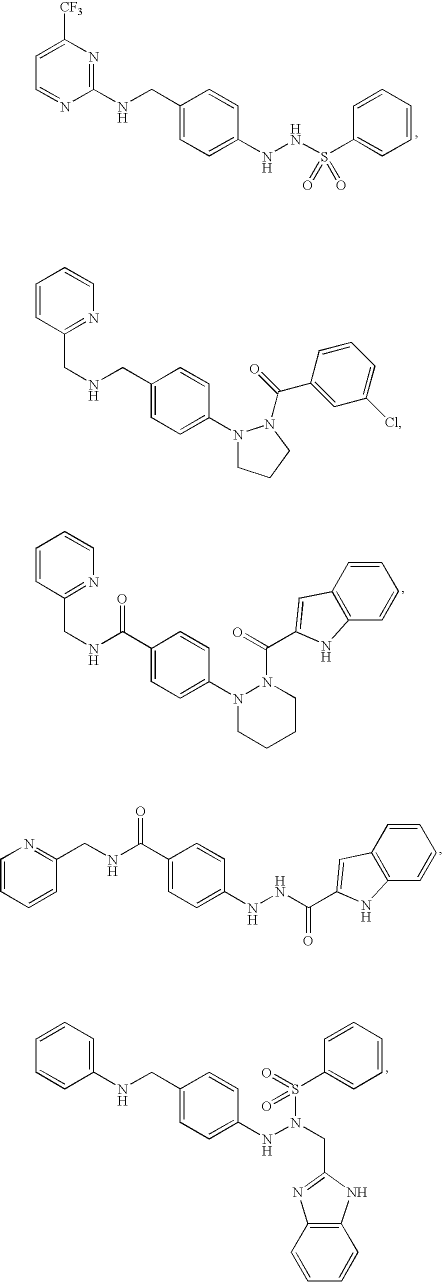 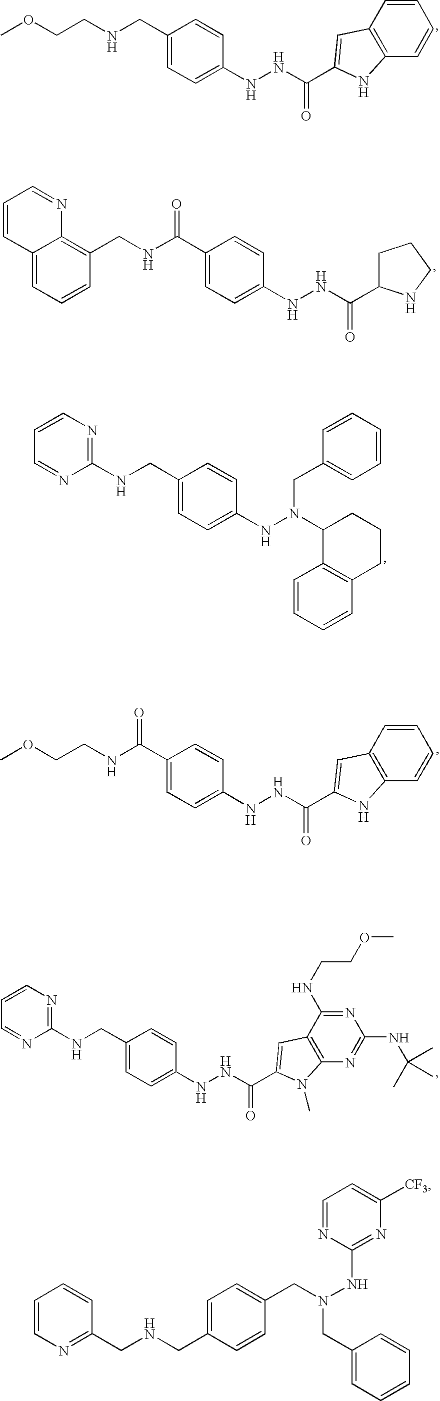 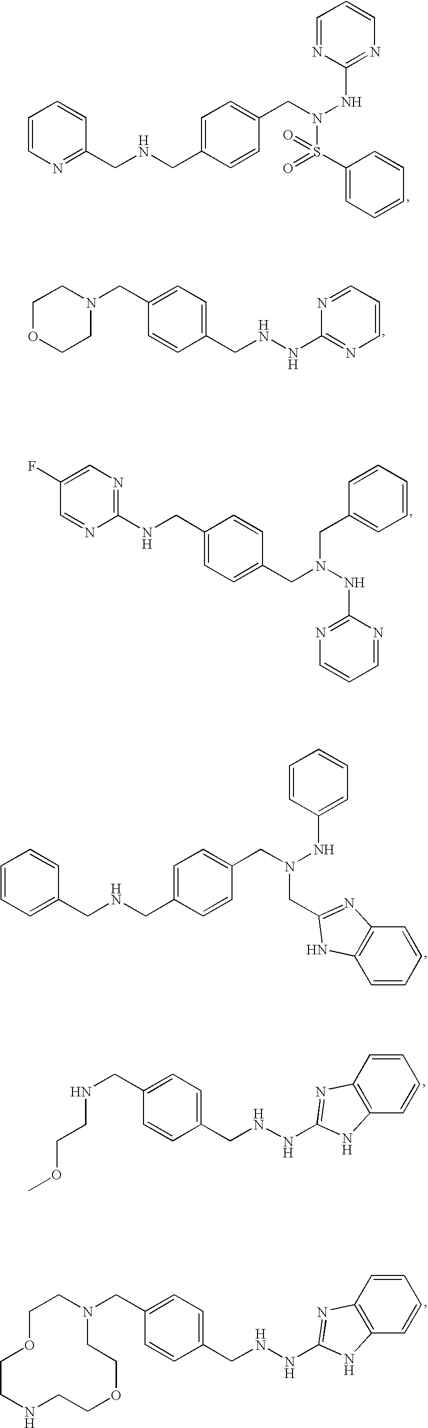 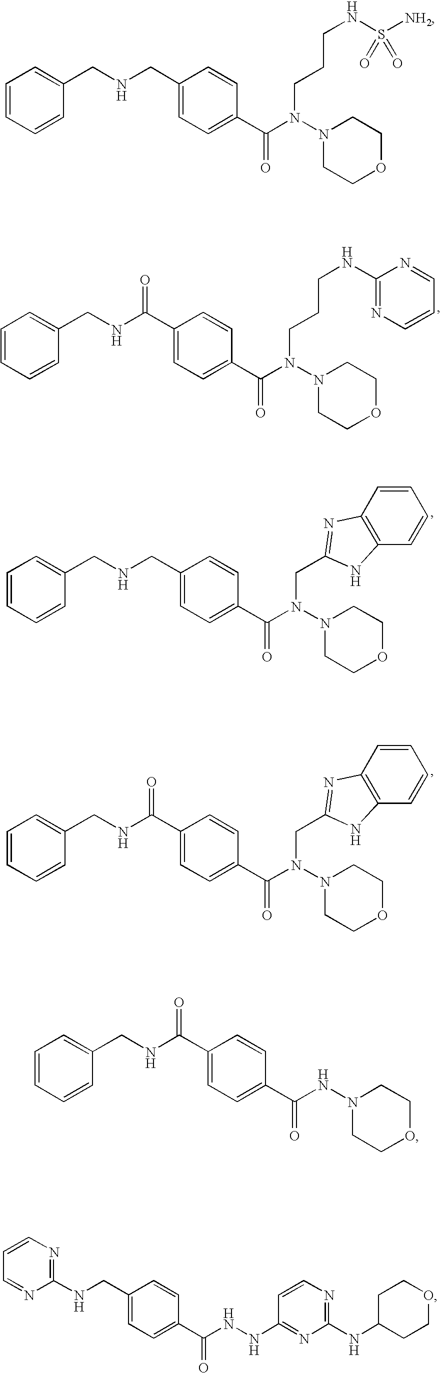 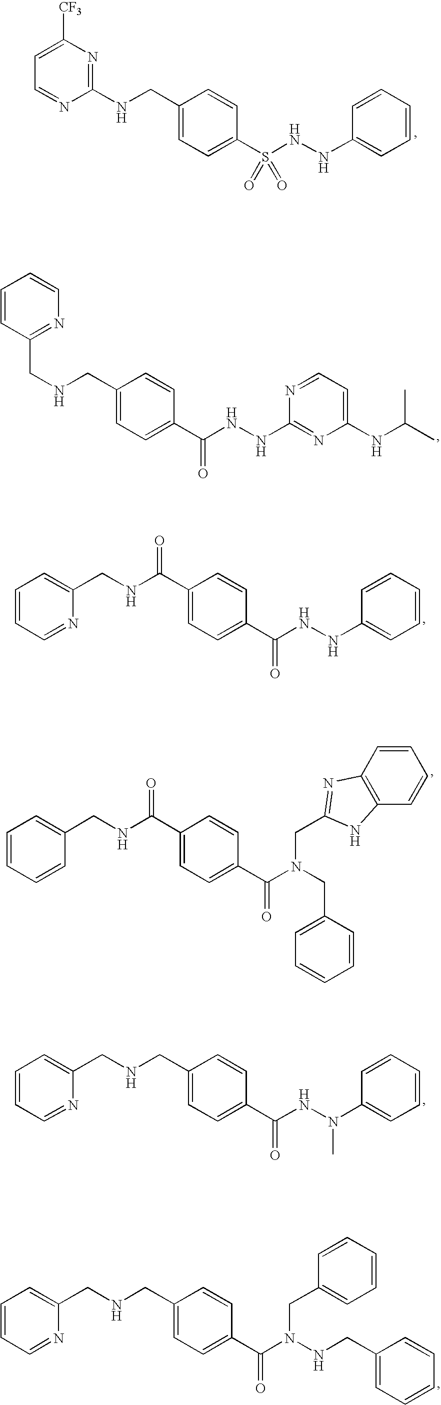 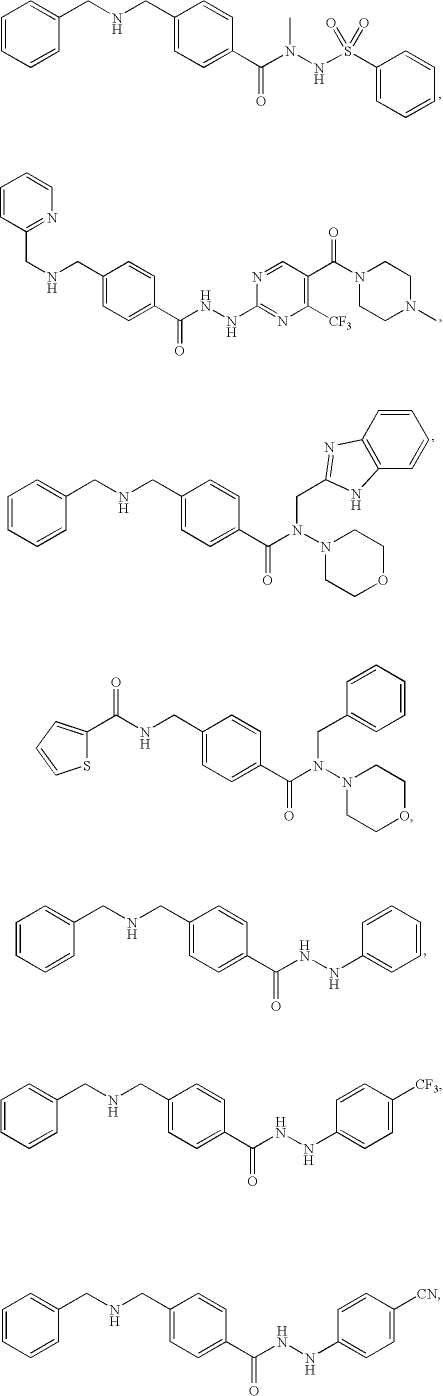 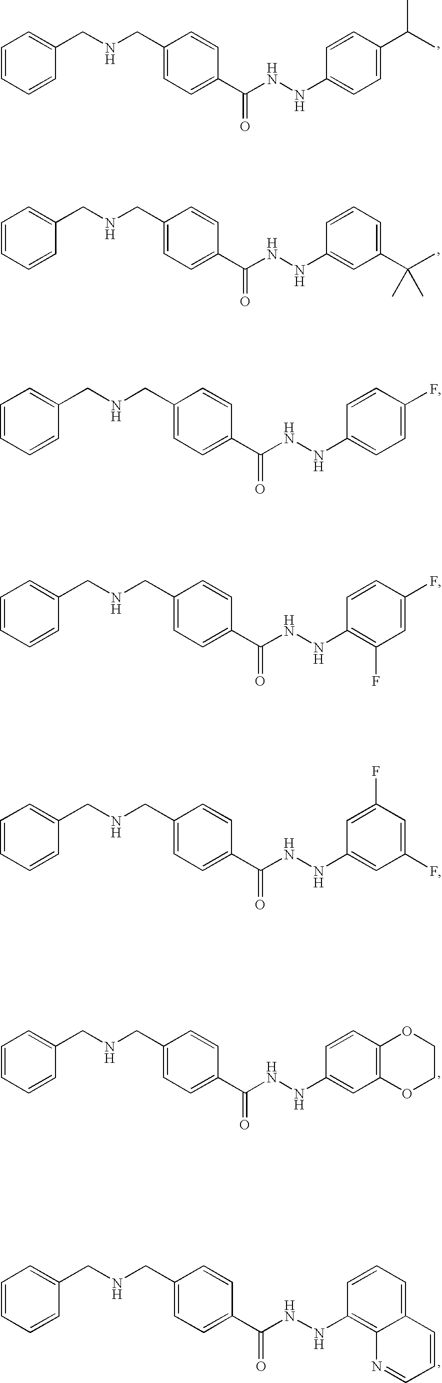 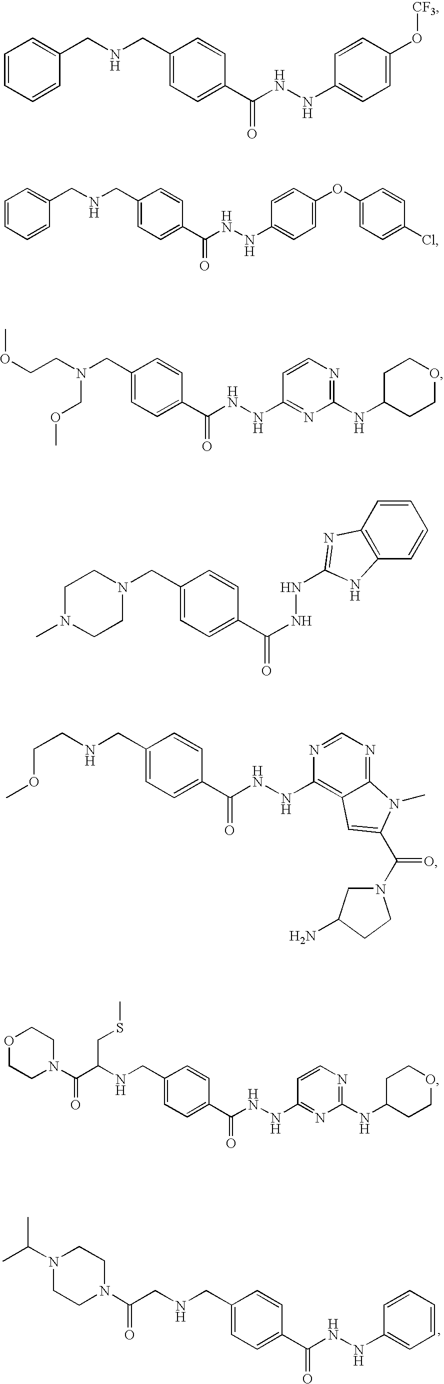 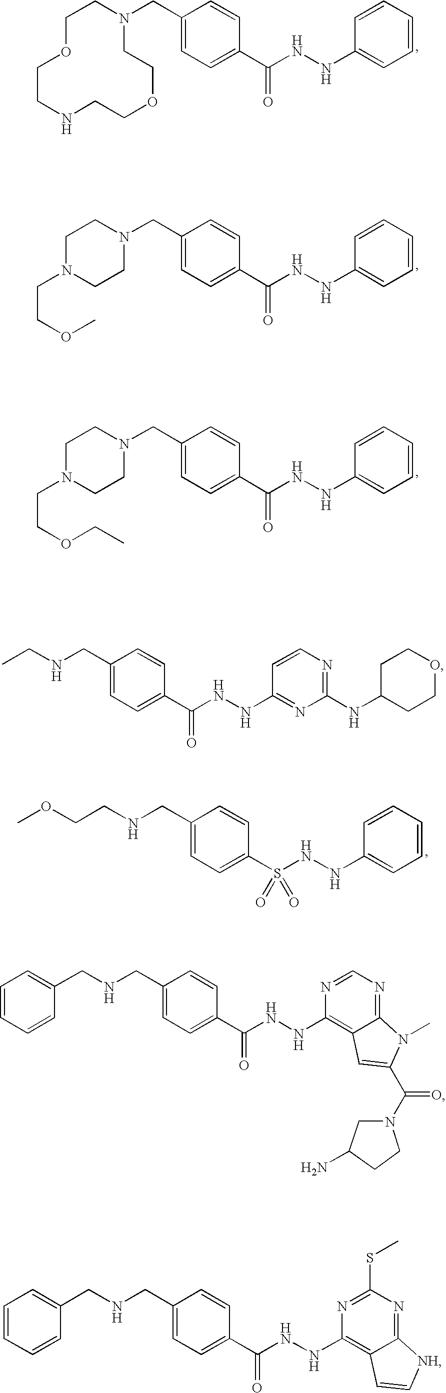 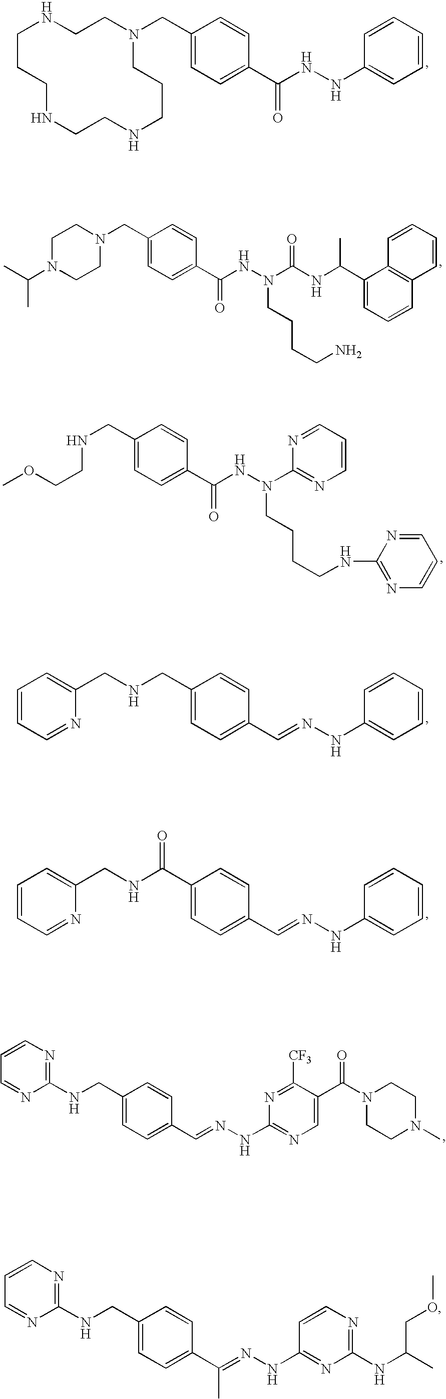 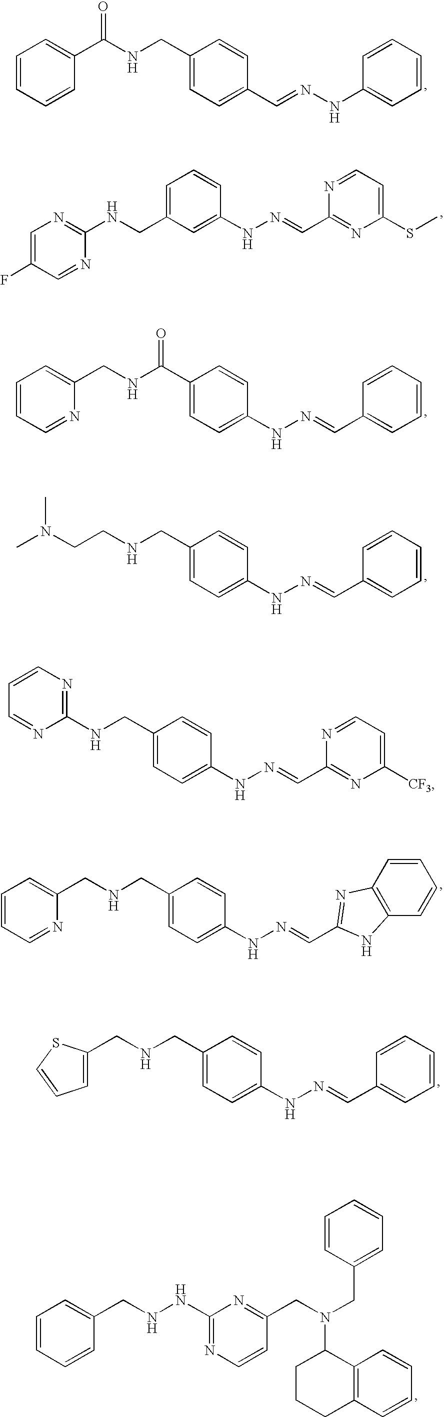 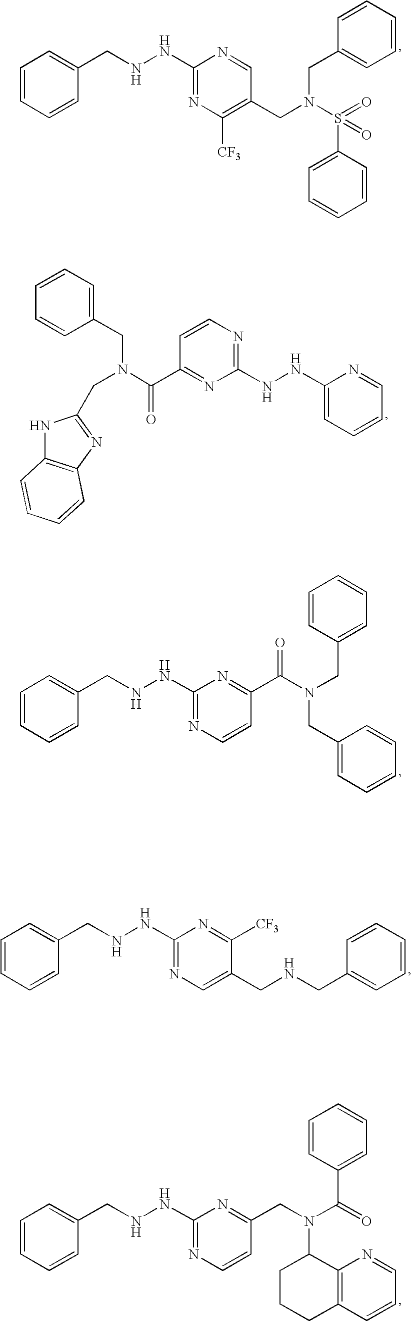 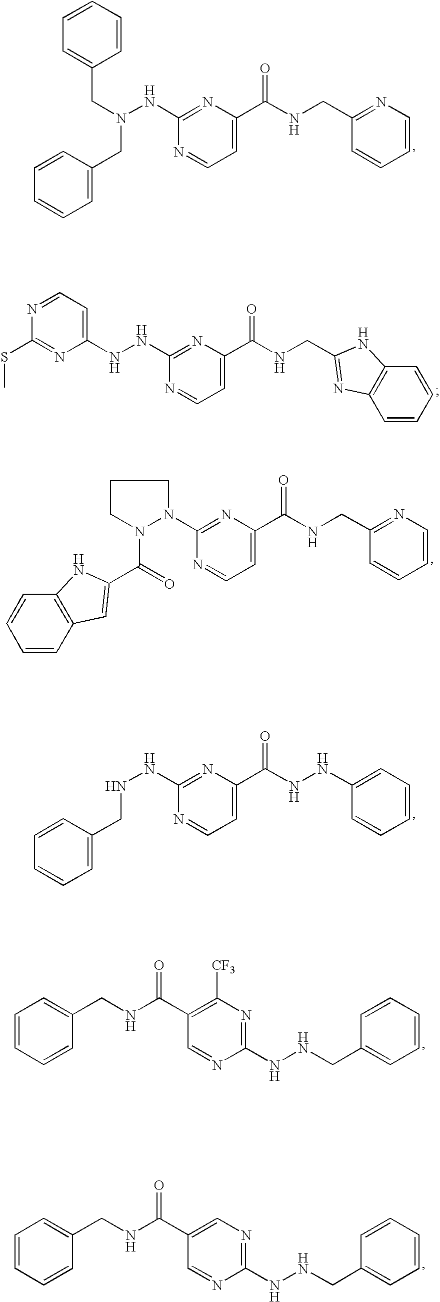 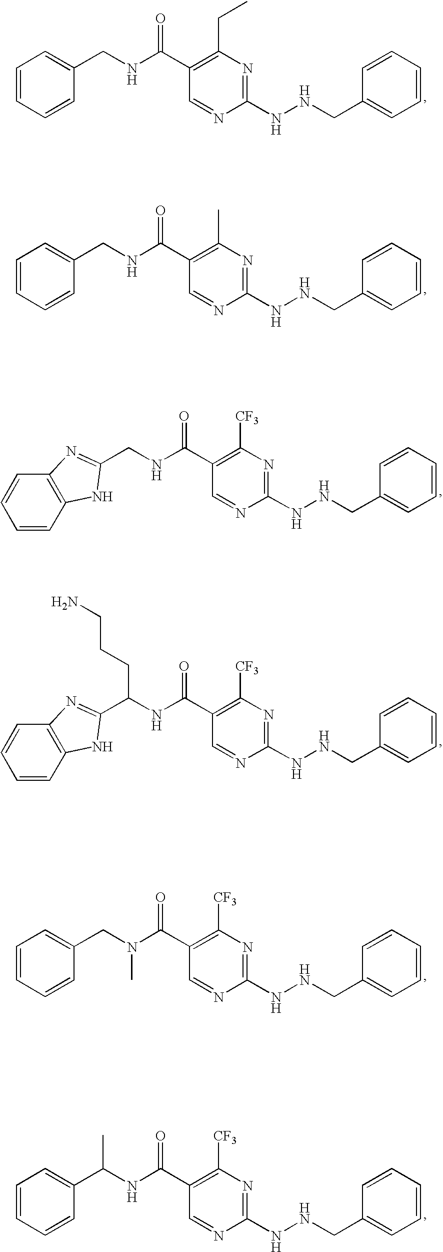 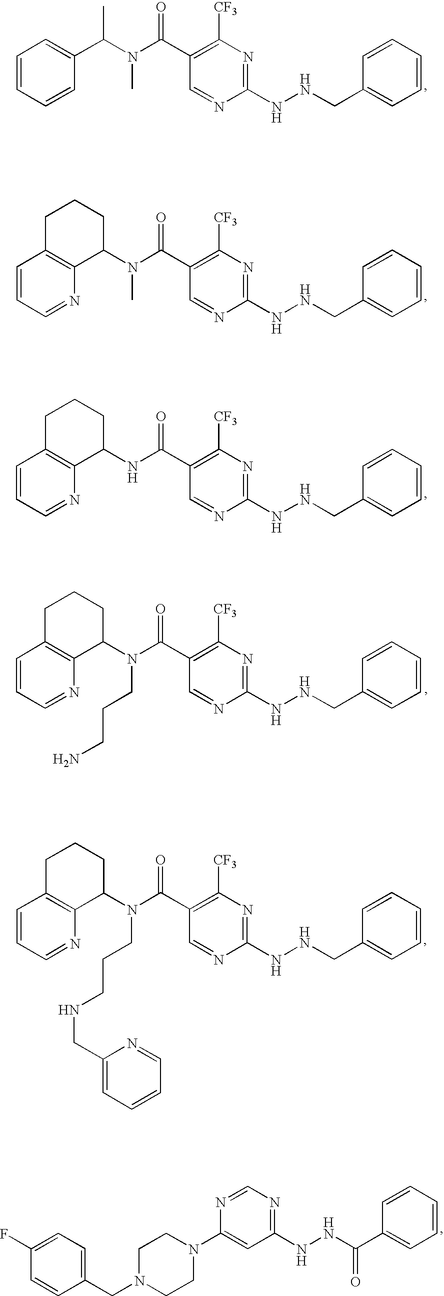 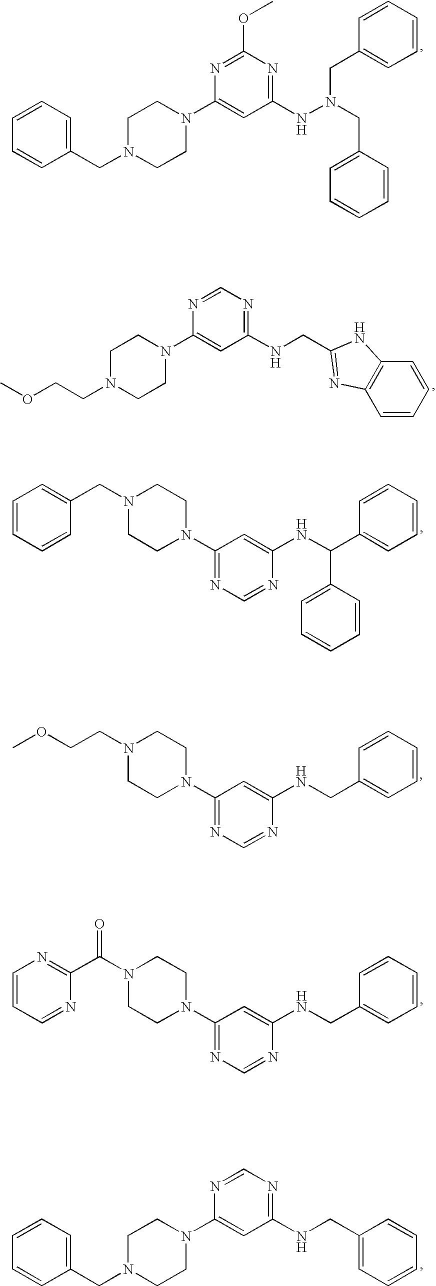 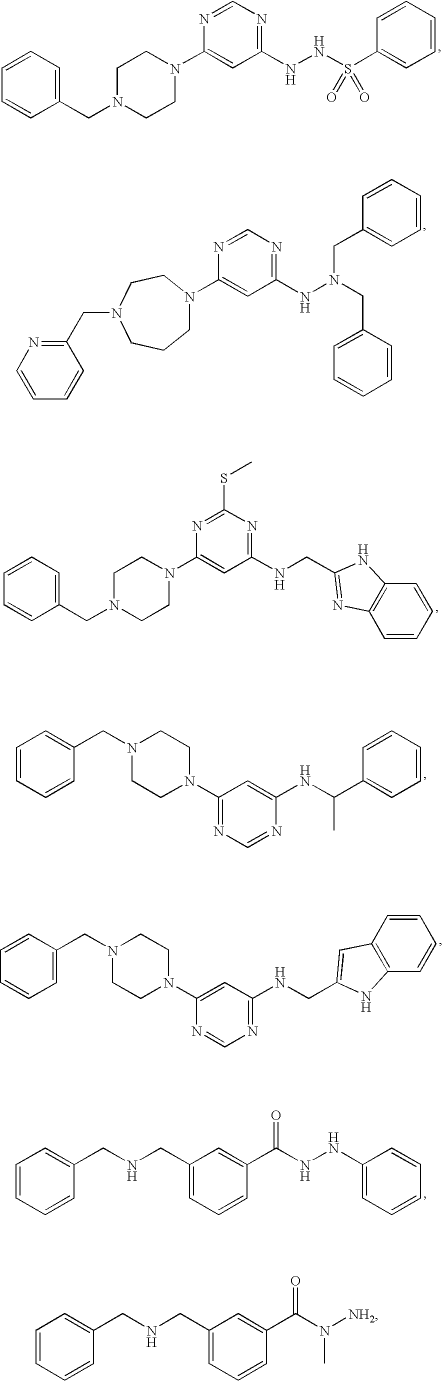 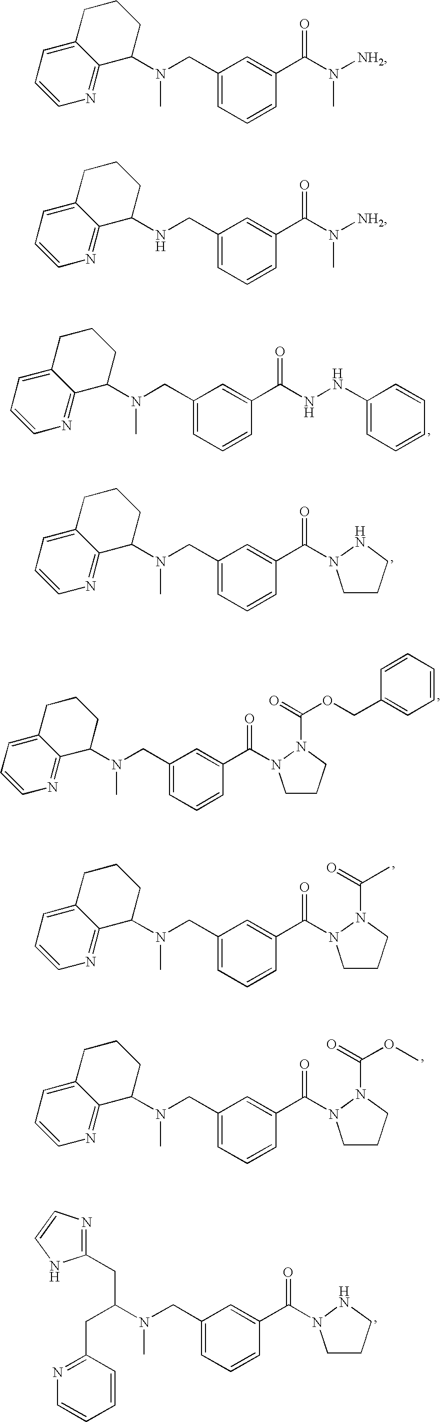 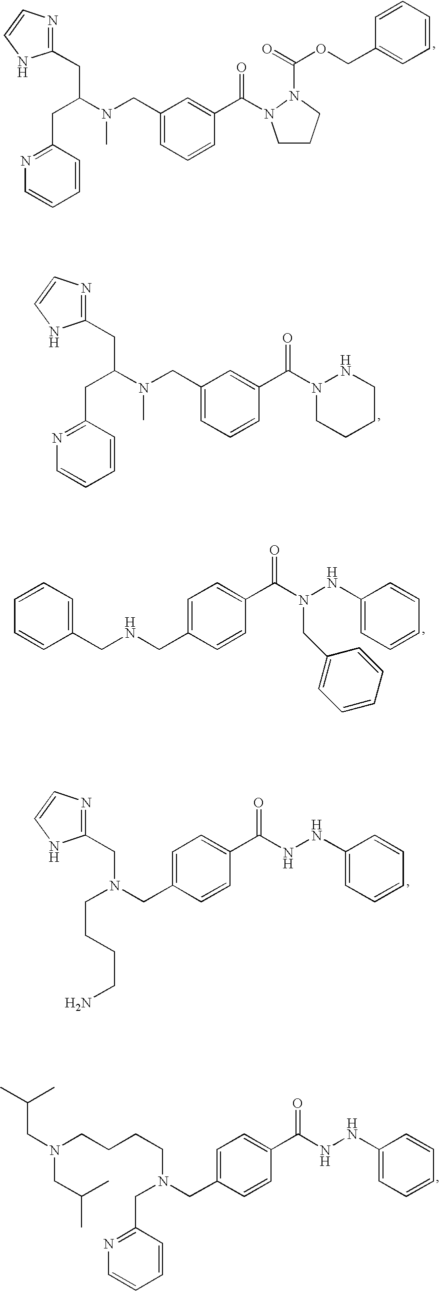 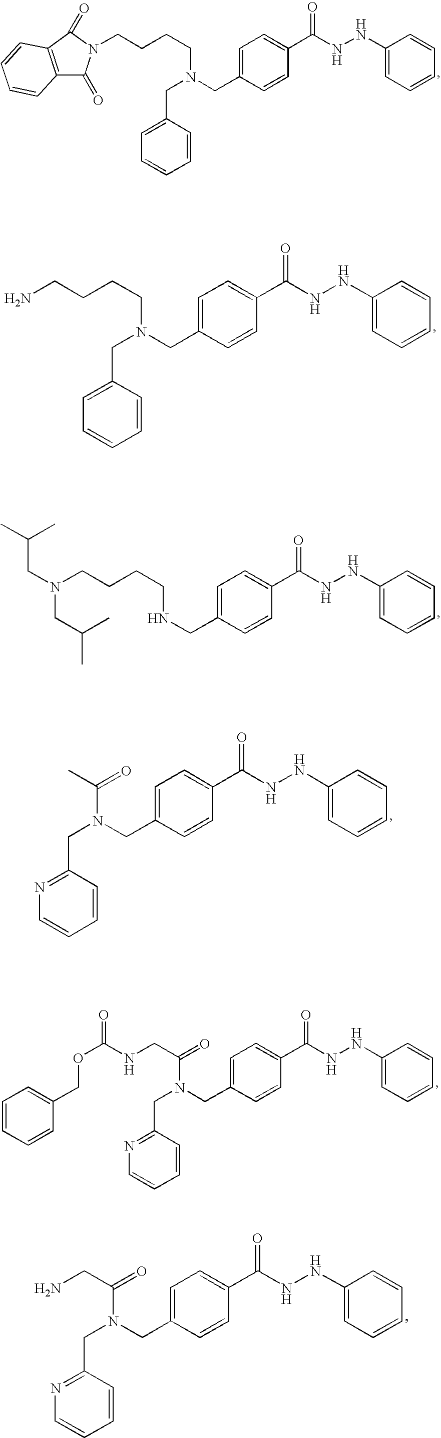 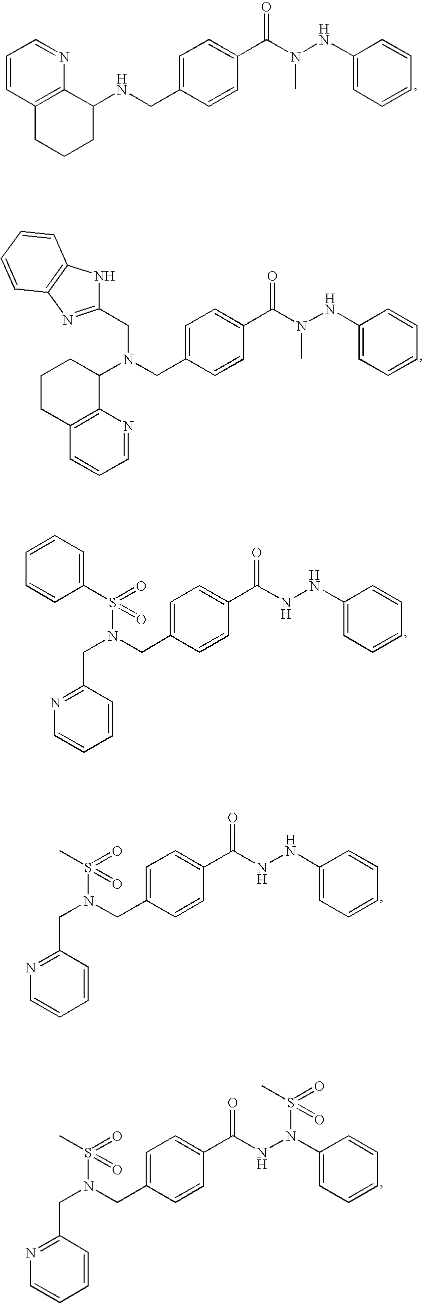 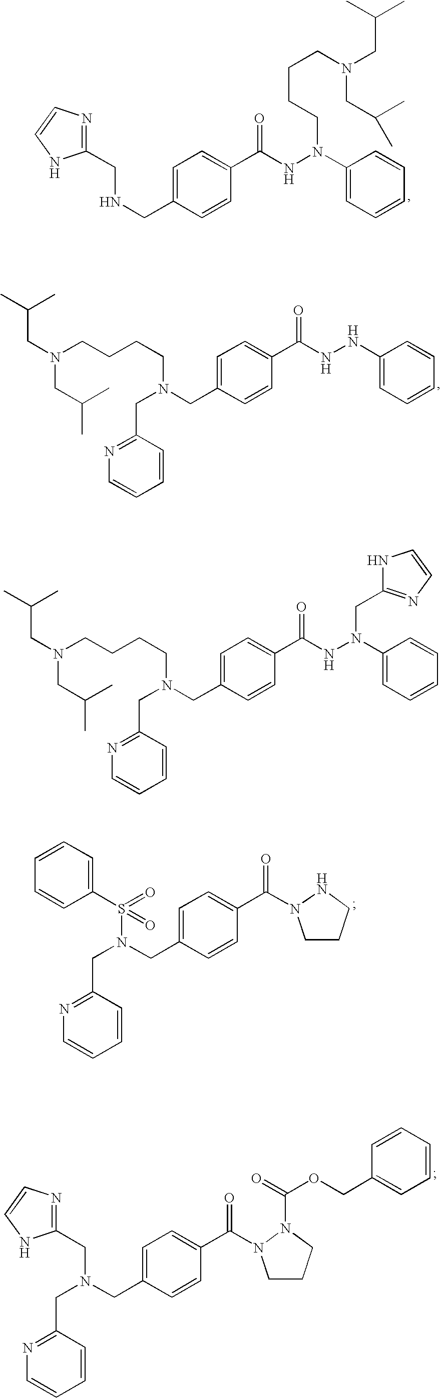 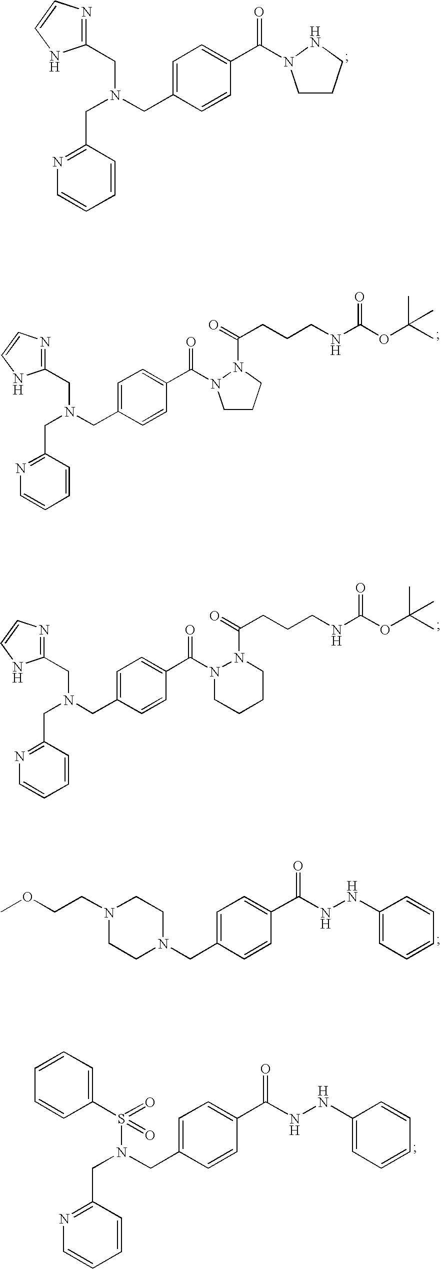 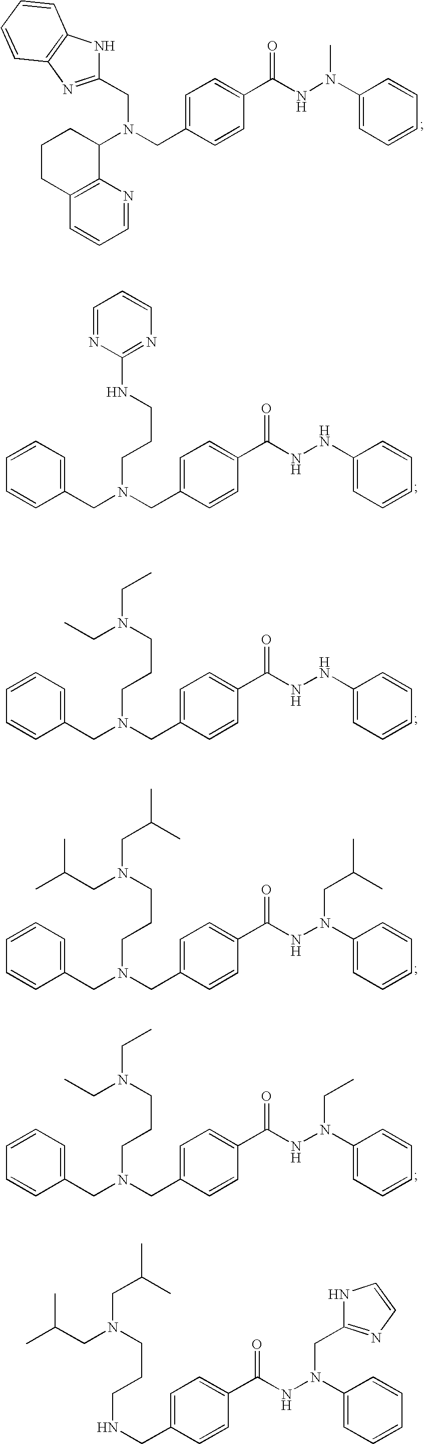
and pharmaceutically acceptable salts, solvates, prodrugs, tautomers, or esters thereof.

31. A composition comprising at least one compound of claim 1, or a pharmaceutically acceptable salt, solvate, prodrug, tautomer, or ester thereof, and a pharmaceutically acceptable excipient.

32. The composition of claim 31, further comprising at least one additional pharmaceutically active compound.

33. The composition of claim 32, wherein the at least one additional pharmaceutically active compound is selected from the group consisting of amprenavir, lamivudine, zidovudine, indinavir, IDV, MK-639, FTC, emtricitabine, lamivudine, 3TC, abacavir, lamivudine, saquinavir, enfuvirtide, T-20, zalcitabine, ddC, dideoxycytidine, saquinavir, SQV, lopinavir, ritonavir, Fosamprenavir Calcium, ABT-538, delavirdine, DLV, AZT, azidothymidine, ZDV, atazanavir sulfate, efavirenz, tenofovir disoproxil, didanosine, ddI, dideoxyinosine, nelfinavir, NFV, nevirapine, BI-RG-587, tenofovir disoproxil fumarate, stavudine, d4T, abacavir, GW5634, (+)Calanolide A, Capravirine, MIV-150, TMC125, RO033-4649, TMC114, Tipranavir, GW640385, Elvucitabine, Alovudine, MIV-210, Racivir, SPD754, Reverset, FP21399, AMD070, GW873140, BMS-488043, PRO 542, TAK-220, TNX-355, UK-427,857, AMD070, BMS-488043, FP21399, GW873140, PRO 542, Schering SCH417690, TAK-220, TNX-355 UK-427,857; Integrase Inhibitors; Maturation Inhibitors, PA457; Zinc Finger Inhibitors, azodicarbonamide; Antisense Drugs, HGTV43, GEM92; Immune Stimulators, Ampligen, IL-2 (Proleukin), Bay 50-4798, Multikine, IR103; Vaccine-Like Treatment, HRG214, DermaVir, VIR201; and pharmaceutically acceptable salts, solvates, and esters thereof.

34. The composition of claim 32, wherein the at least one additional pharmaceutically active compound is selected from the group consisting of 13-cis-Retinoic Acid, 2-Amino-6-Mercaptopurine, 2-CdA, 2-Chlorodeoxyadenosine, 5-fluorouracil, 5-FU, 6-TG, 6-Thioguanine, 6-Mercaptopurine, 6-MP, Accutane, Actinomycin-D, Adriamycin, Adrucil, Agrylin, Ala-Cort, Aldesleukin, Alemtuzumab, Alitretinoin, Alkaban-AQ, Alkeran, All-transretinoic acid, Alpha interferon, Altretamine, Amethopterin, Amifostine, Aminoglutethimide, Anagrelide, Anandron, Anastrozole, Arabinosylcytosine, Ara-C, Aranesp, Aredia, Arimidex, Aromasin, Arsenic trioxide, Asparaginase, ATRA, Avastin, BCG, BCNU, Bevacizumab, Bexarotene, Bicalutamide, BiCNU, Blenoxane, Bleomycin, Bortezomib, Busulfan, Busulfex, C225, Calcium Leucovorin, Campath, Camptosar, Camptothecin-11, Capecitabine, Carac, Carboplatin, Carmustine, Carmustine wafer, Casodex, CCNU, CDDP, CeeNU, Cerubidine, cetuximab, Chlorambucil, Cisplatin, Citrovorum Factor, Cladribine, Cortisone, Cosmegen, CPT-11, Cyclophosphamide, Cytadren, Cytarabine, Cytarabine liposomal, Cytosar-U, Cytoxan, Dacarbazine, Dactinomycin, Darbepoetin alfa, Daunomycin, Daunorubicin, Daunorubicin hydrochloride, Daunorubicin liposomal, DaunoXome, Decadron, Delta-Cortef, Deltasone, Denileukin diftitox, DepoCyt, Dexamethasone, Dexamethason Acetate, dexamethasone sodium phosphate, Dexasone, Dexrazoxane, DHAD, DIC, Diodex, Docetaxel, Doxil, Doxorubicin, Doxorubicin liposomal, Droxia, DTIC, DTIC-Dome, Duralone, Efudex, Eligard, Ellence, Eloxatin, Elspar, Emcyt, Epirubicin, Epoetin alfa, Erbitux, Erwinia-L-asparaginase, Estramustine, Ethyol, Etopophos, Etoposide, Etoposide phosphate, Eulexin, Evista, Exemestane, Fareston, Faslodex, Femara, Filgrastim, Floxuridine, Fludara, Fludarabine, Fluoroplex, Fluorouracil, Fluorouracil(cream), Fluoxyrnesterone, Flutamide, Folinic Acid, FUDR, Fulvestrant, G-CSF, Gefitinib, Gemcitabine, Gemtuzumab ozogamicin, Gemzar, Gleevec, Gliadel wafer, Glivec, GM-CSF, Goserelin, granulocyte colony stimulating factor, Granulocyte macrophage colony stimulating factor, Halotestin, Herceptin, Hexadrol, Hexylen, Hexamethylmelamine, HMM, Hycamtin, Hydrea, Hydrocort Acetate, Hydrocortisone, Hydrocortisone sodium phosphate, Hydrocortisone sodium succinate, Hydrocortone phosphate, Hydroxyurea, Ibritumomab, Ibritumomab Tiuxetan, Idamycin, Idarubicin, Ifex, IFN-alpha, Ifosfamide, IL-2, IL-11, Imatinib mesylate, Imidazole Carboxamide, Interferon alfa, Interferon Alfa-2b (PEG conjugate), Interleukin-2, Interleukin-11, Intron A (interferon alfaL2b), Iressa, Irinotecan, Isotretinoin, Kidrolase, Lanacort, L-asparaginase, LCR, Letrozole, Leucovorin, Leukeran, Leukine, Leuprolide, Leurocristine, Leustatin, Liposomal Ara-C, Liquid Pred, Lomustine, L-PAM, L-Sarcolysin, Lupron, Lupron Depot, Matulane, Maxidex, Mechlorethamine, Mechlorethamine hydrochloride, Medralone, Medrol, Megace, Megestrol, Megestrol Acetate, Melphalan, Mercaptopurine, Mesna, Mesnex, Methotrexate, Methotrexate Sodium, Methylprednisolone, Meticorten, Mitomycin, Mitomycin-C, Mitoxantrone, M-Prednisol, MTC, MTX, Mylocel, Mylotarg, Navelbine, Neosar, Neulasta, Neumega, Neupogen, Nilandron, Nilutamide, Nitrogen Mustard, Novaldex, Novantrone, Octreotide, Octreotide acetate, Oncospar, Oncovin, Ontak, Onxal, Oprevelkin, Orapred, Orasone, Oxaliplatin, Paclitaxel, Pamidronate, Panretin, Paraplatin, Pediapred, PEG Interferon, Pegaspargase, Pegfilgrastim, PEG-INTRON, PEG-L-asparaginase, Phenylalanine Mustard, Platinol, Platinol-AQ, Prednisolone, Prednisone, Prelone, Procarbazine, PROCRIT, Proleukin, Prolifeprospan 20 with Carmustine implant, Purinethol, Raloxifene, Rheumatrex, Rituxan, Rituximab, Roveron-A (interferon α-2a), Rubex, Rubidomycin hydrochloride, Sandostatin, Sandostatin LAR, Sargramostim, Solu-Cortef, Solu-Medrol, STI-571, Streptozocin, Tamoxifen, Targretin, Taxol, Taxotere, Temodar, Temozolomide, Teniposide, TESPA, Thalidomide, Thalomid, TheraCys, Thioguanine, Thioguanine Tabloid, Thiophosphoamide, Thioplex, Thiotepa, TICE, Toposar, Topotecan, Toremifene, Trastuzumab, Tretinoin, Trexall, Trisenox, TSPA, VCR, Velban, Velcade, VePesid, Vesanoid, Viadur, Vinorelbine, Vinorelbine tartrate, VLB, VM-26, VP-16, Vumon, Xeloda, Zanosar, Zevalin, Zinecard, Zoladex, Zoledronic acid, Zometa, and pharmaceutically acceptable salts, solvates, and esters thereof.

35. A method of treating a disorder, symptom or disease in a patient in need of such treatment, comprising administering to the patient an effective amount of at least one compound of claim 1.

36. The method of claim 35, wherein said disorder, symptom or disease is a disorder, symptom or disease that is modulated by chemokine receptor activity or signaling.

37. The method of claim 36 wherein said treating is treatment or prophylaxis and the disorder, symptom or disease that is modulated by chemokine receptor activity or signaling is human immunodeficiency virus infections, flavivirus infections, pestivirus infections or cancer.

38. The method of claim 37, wherein the disorder, symptom or disease that is modulated by chemokine receptor activity or signaling is a cancer selected from the group consisting of bladder cancer, breast cancer, colorectal cancer, endometrial cancer, head & neck cancer, leukemia, lung cancer, lymphoma, melanoma, non-small-cell lung cancer, ovarian cancer, prostate cancer, testicular cancer, uterine cancer, cervical cancer, thyroid cancer, gastric cancer, brain stem glioma, cerebellar astrocytoma, cerebral astrocytoma, ependymoma, Ewing's sarcoma family of tumors, germ cell tumor, extracranial cancer, Hodgkin's disease, leukemia, acute lymphoblastic leukemia, acute myeloid leukemia, liver cancer, medulloblastoma, neuroblastoma, brain tumors generally, non-Hodgkin's lymphoma, ostessarcoma, malignant fibrous histiocytoma of bone, retinoblastoma, rhabdomyosarcoma, soft tissue sarcomas generally, supratentorial primitive neuroectodermal and pineal tumors, visual pathway and hypothalamic glioma, Wilms' tumor, acute lymphocytic leukemia, adult acute myeloid leukemia, adult non-Hodgkin's lymphoma, chronic lymphocytic leukemia, chronic myeloid leukemia, esophageal cancer, hairy cell leukemia, kidney cancer, multiple myeloma, oral cancer, pancreatic cancer, primary central nervous system lymphoma, skin cancer, and small-cell lung cancer.

39. The method of claim 37, further comprising administering at least one additional pharmaceutically active compound is selected from the group consisting of amprenavir, lamivudine, zidovudine, indinavir, IDV, MK-639, FTC, emtricitabine, lamivudine, 3TC, abacavir, lamivudine, saquinavir, enfuvirtide, T-20, zalcitabine, ddC, dideoxycytidine, saquinavir, SQV, lopinavir, ritonavir, Fosamprenavir Calcium, ABT-538, delavirdine, DLV, AZT, azidothymidine, ZDV, atazanavir sulfate, efavirenz, tenofovir disoproxil, didanosine, ddI, dideoxyinosine, nelfinavir, NFV, nevirapine, BI-RG-587, tenofovir disoproxil fumarate, stavudine, d4T, abacavir, GW5634, (+)Calanolide A, Capravirine, MIV-150, TMC125, RO033-4649, TMC114, Tipranavir, GW640385, Elvucitabine, Alovudine, MIV-210, Racivir, SPD754, Reverset, FP21399, AMD070, GW873140, BMS-488043, PRO 542, TAK-220, TNX-355, UK-427,857, AMD070, BMS-488043, FP21399, GW873140, PRO 542, Schering SCH417690, TAK-220, TNX-355 UK-427,857; Integrase Inhibitors; Maturation Inhibitors, PA457; Zinc Finger Inhibitors, azodicarbonamide; Antisense Drugs, HGTV43, GEM92; Immune Stimulators, Ampligen, IL-2 (Proleukin), Bay 50-4798, Multikine, IR103; Vaccine-Like Treatment, HRG214, DermaVir, VIR201; and pharmaceutically acceptable salts, solvates, and esters thereof.

40. The method of claim 38, further comprising administering at least one additional pharmaceutically active compound is selected from the group consisting of 13-cis-Retinoic Acid, 2-Amino-6-Mercaptopurine, 2-CdA, 2-Chlorodeoxyadenosine, 5-fluorouracil, 5-FU, 6-TG, 6-Thioguanine, 6-Mercaptopurine, 6-MP, Accutane, Actinomycin-D, Adriamycin, Adrucil, Agrylin, Ala-Cort, Aldesleukin, Alemtuzumab, Alitretinoin, Alkaban-AQ, Alkeran, All-transretinoic acid, Alpha interferon, Altretamine, Amethopterin, Amifostine, Aminoglutethimide, Anagrelide, Anandron, Anastrozole, Arabinosylcytosine, Ara-C, Aranesp, Aredia, Arimidex, Aromasin, Arsenic trioxide, Asparaginase, ATRA, Avastin, BCG, BCNU, Bevacizumab, Bexarotene, Bicalutamide, BiCNU, Blenoxane, Bleomycin, Bortezomib, Busulfan, Busulfex, C225, Calcium Leucovorin, Campath, Camptosar, Camptothecin-11, Capecitabine, Carac, Carboplatin, Carmustine, Carmustine wafer, Casodex, CCNU, CDDP, CeeNU, Cerubidine, cetuximab, Chlorambucil, Cisplatin, Citrovorum Factor, Cladribine, Cortisone, Cosmegen, CPT-11, Cyclophosphamide, Cytadren, Cytarabine, Cytarabine liposomal, Cytosar-U, Cytoxan, Dacarbazine, Dactinomycin, Darbepoetin alfa, Daunomycin, Daunorubicin, Daunorubicin hydrochloride, Daunorubicin liposomal, DaunoXome, Decadron, Delta-Cortef, Deltasone, Denileukin diftitox, DepoCyt, Dexamethasone, Dexamethason Acetate, dexamethasone sodium phosphate, Dexasone, Dexrazoxane, DHAD, DIC, Diodex, Docetaxel, Doxil, Doxorubicin, Doxorubicin liposomal, Droxia, DTIC, DTIC-Dome, Duralone, Efudex, Eligard, Ellence, Eloxatin, Elspar, Emcyt, Epirubicin, Epoetin alfa, Erbitux, Erwinia-L-asparaginase, Estramustine, Ethyol, Etopophos, Etoposide, Etoposide phosphate, Eulexin, Evista, Exemestane, Fareston, Faslodex, Femara, Filgrastim, Floxuridine, Fludara, Fludarabine, Fluoroplex, Fluorouracil, Fluorouracil(cream), Fluoxyrnesterone, Flutamide, Folinic Acid, FUDR, Fulvestrant, G-CSF, Gefitinib, Gemcitabine, Gemtuzumab ozogamicin, Gemzar, Gleevec, Gliadel wafer, Glivec, GM-CSF, Goserelin, granulocyte colony stimulating factor, Granulocyte macrophage colony stimulating factor, Halotestin, Herceptin, Hexadrol, Hexylen, Hexamethylmelamine, HMM, Hycamtin, Hydrea, Hydrocort Acetate, Hydrocortisone, Hydrocortisone sodium phosphate, Hydrocortisone sodium succinate, Hydrocortone phosphate, Hydroxyurea, Ibritumomab, Ibritumomab Tiuxetan, Idamycin, Idarubicin, Ifex, IFN-alpha, Ifosfamide, IL-2, IL-11, Imatinib mesylate, Imidazole Carboxamide, Interferon alfa, Interferon Alfa-2b (PEG conjugate), Interleukin-2, Interleukin-11, Intron A (interferon alfaL2b), Iressa, Irinotecan, Isotretinoin, Kidrolase, Lanacort, L-asparaginase, LCR, Letrozole, Leucovorin, Leukeran, Leukine, Leuprolide, Leurocristine, Leustatin, Liposomal Ara-C, Liquid Pred, Lomustine, L-PAM, L-Sarcolysin, Lupron, Lupron Depot, Matulane, Maxidex, Mechlorethamine, Mechlorethamine hydrochloride, Medralone, Medrol, Megace, Megestrol, Megestrol Acetate, Melphalan, Mercaptopurine, Mesna, Mesnex, Methotrexate, Methotrexate Sodium, Methylprednisolone, Meticorten, Mitomycin, Mitomycin-C, Mitoxantrone, M-Prednisol, MTC, MTX, Mylocel, Mylotarg, Navelbine, Neosar, Neulasta, Neumega, Neupogen, Nilandron, Nilutamide, Nitrogen Mustard, Novaldex, Novantrone, Octreotide, Octreotide acetate, Oncospar, Oncovin, Ontak, Onxal, Oprevelkin, Orapred, Orasone, Oxaliplatin, Paclitaxel, Pamidronate, Panretin, Paraplatin, Pediapred, PEG Interferon, Pegaspargase, Pegfilgrastim, PEG-INTRON, PEG-L-asparaginase, Phenylalanine Mustard, Platinol, Platinol-AQ, Prednisolone, Prednisone, Prelone, Procarbazine, PROCRIT, Proleukin, Prolifeprospan 20 with Carmustine implant, Purinethol, Raloxifene, Rheumatrex, Rituxan, Rituximab, Roveron-A (interferon α-2a), Rubex, Rubidomycin hydrochloride, Sandostatin, Sandostatin LAR, Sargramostim, Solu-Cortef, Solu-Medrol, STI-571, Streptozocin, Tamoxifen, Targretin, Taxol, Taxotere, Temodar, Temozolomide, Teniposide, TESPA, Thalidomide, Thalomid, TheraCys, Thioguanine, Thioguanine Tabloid, Thiophosphoamide, Thioplex, Thiotepa, TICE, Toposar, Topotecan, Toremifene, Trastuzumab, Tretinoin, Trexall, Trisenox, TSPA, VCR, Velban, Velcade, VePesid, Vesanoid, Viadur, Vinorelbine, Vinorelbine tartrate, VLB, VM-26, VP-16, Vumon, Xeloda, Zanosar, Zevalin, Zinecard, Zoladex, Zoledronic acid, Zometa, and pharmaceutically acceptable salts, solvates, and esters thereof.
